(19) United States
(12) Patent Application Publication
(10) Pub. No.: US 2026/0297109 A1
(43) Pub. Date: Oct. 1, 2026

(54) COMPOUNDS AS SUMO ACTIVATING ENZYME INHIBITORS

(71) Applicant: WIGEN BIOMEDICINE TECHNOLOGY (SHANGHAI) CO., LTD., Shanghai (CN)

(72) Inventors: Yuli XIE, Shanghai (CN); Yingming WU, Shanghai (CN); Lihui QIAN, Shanghai (CN)

(21) Appl. No.: 19/127,545

(22) PCT Filed: Nov. 10, 2023
(86) PCT No.: PCT/CN2023/131049

§ 371 Date: May 6, 2025

(30) Foreign Application Priority Data
Nov. 11, 2022 (CN) 202211420715.3
Aug. 15, 2023 (CN) 202311026790.6
Sep. 5, 2023 (CN) 202311138646.1

(51) Int. Cl. C07D 495/10; A61K 31/506; C07B 59/00; C07D 409/14; C07D 471/04; C07D 495/20; C07D 495/22; C07D 513/10; C07D 513/20

(52) U.S. Cl. CPC C07D 495/10; A61K 31/506; C07B 59/002; C07D 409/14; C07D 471/04; C07D 495/20; C07D 495/22; C07D 513/10; C07D 513/20; C07B 2200/05

(57) ABSTRACT

Disclosed in the present disclosure are compounds as SUMO activating enzyme inhibitors. Particularly, the present disclosure relates to compounds as represented by general formula (1) and preparation methods therefor, and uses of the compounds of general formula (1) and isomers thereof, crystalline forms thereof, pharmaceutically acceptable salts thereof, hydrates thereof or solvates thereof as SAE inhibitors. The compounds and the isomers thereof, the crystalline forms thereof, the pharmaceutically acceptable salts thereof, the hydrates thereof, or the solvates thereof of the present disclosure can be used in preparing a medicament for treating or preventing a disease related to SAE protein.

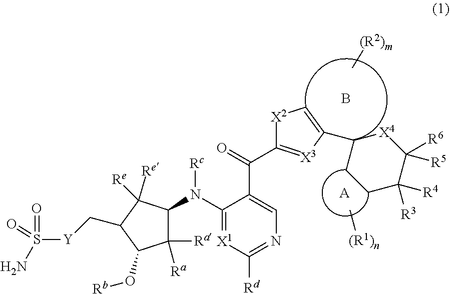

The present application claims priority to Chinese Patent Application No. 202211420715.3 filed on Nov. 11, 2022, Chinese Patent Application No. 202311026790.6 filed on Aug. 15, 2023, and Chinese Patent Application No. 202311138646.1 filed on Sep. 5, 2023, which are incorporated herein by reference in their entirety.

### TECHNICAL FIELD

The present disclosure relates to the field of pharmaceutical chemistry, and particularly relates to a class of compounds with an inhibitory effect on SAE protein, a preparation method therefor, and use of the compounds in preparing a medicament for treating or preventing a related disease mediated by SAE.

### BACKGROUND

Small ubiquitin-like modifiers (SUMOs) are a family of ubiquitin-like proteins that function as reversible post-translational modifiers within cells. Mammalian cells express three types of SUMO family proteins: SUMO1, SUMO2, and SUMO3. SUMO2 shares about 95% amino acid sequence homology with SUMO3 and primarily forms oligomeric chains upon protein modification. SUMO1 shares about 45% sequence homology with SUMO2 and SUMO3, and primarily modifies proteins in a monomer form. Sumoylation of a target protein involves a three-step enzyme-catalyzed reaction that activates, transfers, and ultimately conjugates the SUMO protein to the lysine residue of the target protein. The first step is catalyzed by SAE (SUMO activating enzyme), which is a class of activating enzymes known as E1 enzymes and functions as a heterodimer composed of SAE1 and SAE2/UBA2. SAE uses ATP to adenylate the C-terminal glycine residue of SUMO, leading to the formation of a thioester intermediate between the C-terminal glycine of SUMO and a cysteine residue in SAE2. Subsequently, the SUMO protein is transferred from E1 to the SUMO specific conjugating enzyme, collectively known as E2, via thioester bond exchange. Finally, under the action of a SUMO specific E3 protein ligase, the SUMO protein is ultimately conjugated to the lysine residue of the target protein, forming an oligomeric chain. Sumoylation of proteins affects catalytic activity, intracellular localization, and interactions with other proteins. In addition, recent studies have shown that sumoylation of proteins plays a significant role in various cellular signaling pathways, including cell division, DNA repair, chromosome segregation, nuclear transport, gene transcription, and immune regulation. High expression of proteins related to the SUMO signaling pathway is associated with poor prognosis in certain cancer patients. Knockdown of SAE has shown synthetic lethality in certain tumor cells highly expressing MYC. In addition, sumoylation can also modulate innate immune responses. Inhibition of sumoylation can enhance type 1 interferon (IFN) expression. In summary, there is an urgent need to study and identify compounds with potent activity targeting SAE.

### SUMMARY

The present disclosure provides a compound of general formula (1) or an isomer thereof, a crystalline form thereof, a pharmaceutically acceptable salt thereof, a hydrate thereof, or a solvate thereof:

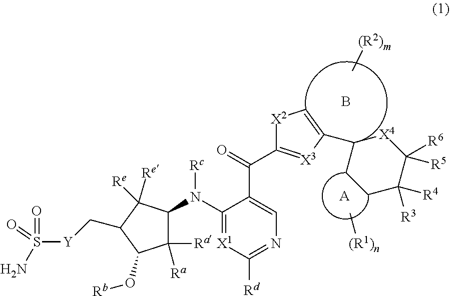

wherein in general formula (1):
Y is —O—, —CH₂—, or —N(H)—;
R^{a} is —H, —F, —NH₂, or —OH;
R^{a′} is —H or —F, and when R^{a} is —NH₂ or —OH, R^{a′} is —H;
R^{b} is —H or (C1-C4) alkyl;
R^{c} is —H or (C1-C4) alkyl;
R^{d} is —H, halogen, —CF₃, or (C1-C4) alkyl;
R^{e} and R^{e′} are each independently and optionally —H or halogen, and R^{e} and R^{e′} are not both —H;
X¹ is C(H), C(F), or N;
X² is S or O;
X³ is C(R^{x3}) or N;
R^{x3} is —H, halogen, or —CH₃;
X⁴ is S, O, C(R^{x41})(R^{x41′}), or N(R^{x42});
R^{x42} is —H, (C1-C4) alkyl or (C3-C5) cycloalkyl;
R^{x41} and R^{x41′} are each independently and optionally —H, halogen, —OH, —OR^{x411}, —N(R^{x411})(R^{x412}), —CN, (C1-C6) alkyl, (C1-C6) haloalkyl, (C3-C9) cycloalkyl, or (C1-C6) alkoxy;
R^{x411} and R^{x412} are each independently and optionally —H, (C1-C4) alkyl, or (C3-C5) cycloalkyl, or R^{x411} and
R^{x412} on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H or halogen;
R³ and R⁴ are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR³¹, —(CH₂)ᵣNR³¹R³², OR³¹, NR³¹R³², —CN, —C(O)NR³¹R³², —NR³²C(O)R³¹, —NR³²S(O)₂R³¹, —S(O)ₚR³¹, and —S(O)₂NR³¹R³²; or R³ and R⁴, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or R³ and the adjacent R⁵, together with the atom to which they are attached, can form a (C3-C9) cycloalkyl or (3- to 11-membered) heterocycloalkyl, wherein the (C3-C9) cycloalkyl or (3- to 11-membered) heterocycloalkyl may be each independently and optionally substituted with 1, 2, 3, or 4 of the following groups: —H, halogen, (C1-C6) alkyl, or (C1-C6) alkoxy; or when R³ and the adjacent R⁵ are both absent, an endocyclic double bond may be formed between the carbon atoms separately linked to R⁴ and R⁶; or R³ and R⁴ together form an oxo; R⁵ and R⁶ are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR³¹, —(CH₂)ᵣNR³¹R³², OR³¹, NR³¹R³², —CN, —C(O)NR³¹R³², —NR³²C(O)R³¹, —NR³²S(O)₂R³¹, —S(O)ₚR³¹, and —S(O)₂NR³¹R³²; or R⁵ and R⁶, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or R⁵ and R⁶ together form an oxo;
ring A is (C6-C10) aryl or (5- to 10-membered) heteroaryl;
each R¹ is independently and optionally: —H, halogen, —OH, —NO₂, —NR³¹R³², —(CH₂)ᵣOR³¹, —(CH₂)ᵣNR³¹R³², —CN, (C1-C6) alkyl, (C1-C6) haloalkyl, (C1-C6) alkoxy, (C2-C6) alkenyl, (C2-C6) alkynyl, (C3-C8) cycloalkyl, —C(O)NR³¹R³², —NR³²C(O)R³¹, —NR³²S(O)₂R³¹, —S(O)ₚR³¹, or —S(O)₂NR³¹R³², wherein the (C1-C6) alkyl, (C1-C6) haloalkyl, (C1-C6) alkoxy, (C2-C6) alkenyl, (C2-C6) alkynyl, or (C3-C8) cycloalkyl may be each independently and optionally substituted with 1, 2, 3, or 4 of the following groups: —H, halogen, —OH, —(CH₂)ᵣOR³¹, —(CH₂)ᵣNR³¹R³², —OR³¹, —NR³¹R³², —CN, or (C1-C6) alkyl;
ring B is (C5-C7) cycloalkyl or (5- to 7-membered) heterocycloalkyl;
each R² is independently and optionally: —H, halogen, —OH, —NR³¹R³², —CN, (C1-C6) alkyl, (C1-C6) haloalkyl, (C1-C6) alkoxy, (C2-C6) alkenyl, (C2-C6) alkynyl, or (C3-C8) cycloalkyl; or two R² on a same carbon atom, together with the carbon atom to which they are attached, can form (4- to 6-membered) heterocycloalkyl or (C3-C6) cycloalkyl, wherein the (4- to 6-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or two R² on a same carbon atom together form an oxo;
R³¹ and R³² are each independently and optionally —H, (C1-C4) alkyl, or (C3-C5) cycloalkyl, or R³¹ and R³² on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H and halogen; and
n is an integer of 0, 1, 2, 3, or 4, m is an integer of 0, 1, 2, 3, or 4, r is an integer of 0, 1, or 2, and p is an integer of 0, 1, or 2.

In another preferred embodiment, in general formula (1), R^{d} is —H, —F, —CF₃, or —CH₃; R^{d} is preferably —H or —F; R^{d} is more preferably —H; R^{d} is more preferably —F.

In another preferred embodiment, in general formula (1), R^{e} and R^{e′} are each independently and optionally —H or —F, and R^{e} and R^{e′} are not both —H.

In another preferred embodiment, in general formula (1), R^{x42} is —H, (C1-C3) alkyl, or (C3-C5) cycloalkyl.

In another preferred embodiment, in general formula (1), R^{x42} is —H,

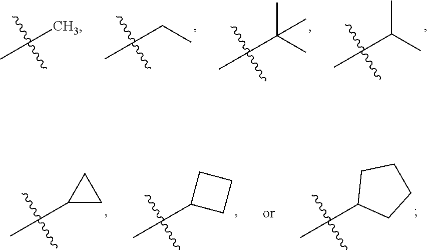

R^{x42} is preferably —H.

In another preferred embodiment, in general formula 1), R^{x41} and R^{x41′} are each independently and optionally —H, —F, —OH, —OCH₃, —N(CH₃)₂, —NH₂, —CN,

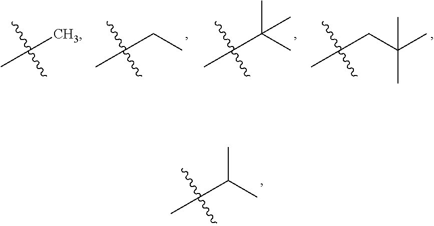

—CF₃, —CH₂CF₃,

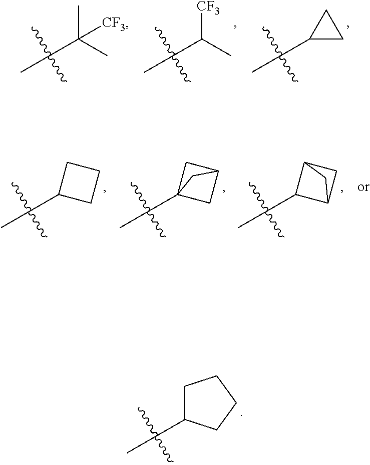

In another preferred embodiment, in general formula (1), R³ and R⁴ are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R³ and R⁴, together with the carbon atom to which they are attached, can form (4- to 6-membered) heterocycloalkyl or (C3-C4) cycloalkyl, wherein the (4- to 6-membered) heterocycloalkyl or (C3-C4) cycloalkyl may be optionally substituted with 1, 2, 3, or 4 of the following groups: —H, —F,

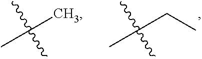

or —OCH₃; or R³ and the adjacent R⁵, together with the atom to which they are attached, form a (C3-C6) cycloalkyl or (3- to 6-membered) heterocycloalkyl, wherein the (C3-C6) cycloalkyl or (3- to 6-membered) heterocycloalkyl may be each independently and optionally substituted with 1, 2, 3, or 4 of the following groups: —H, —F,

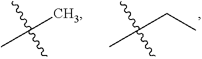

or —OCH₃; or when R³ and the adjacent R⁵ are both absent, an endocyclic double bond may be formed between the carbon atoms separately linked to R⁴ and R⁶; or R³ and R⁴ together form an oxo.

In another preferred embodiment, in general formula (1), R³ and R⁴ are each independently and optionally —H, -D,

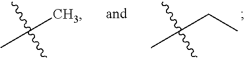

or R³ and R⁴, together with the carbon atom to which they are attached, can form a cyclopropyl; or R³ and the adjacent R⁵, together with the atom to which they are attached, form a cyclopropyl; or R³ and R⁴ together form an oxo.

In another preferred embodiment, in general formula (1), R⁵ and R⁶ are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R⁵ and R⁶, together with the carbon atom to which they are attached, can form a (4- to 6-membered) heterocycloalkyl or (C3-C4) cycloalkyl, wherein the (4- to 6-membered) heterocycloalkyl or (C3-C4) cycloalkyl may be optionally substituted with 1, 2, 3, or 4 of the following groups: —H, —F,

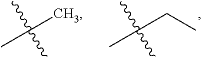

or —OCH₃; or R⁵ and R⁶ together form an oxo.

In another preferred embodiment, in general formula (1), R⁵ and R⁶ are each independently and optionally —H, -D,

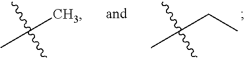

or R⁵ and R⁶, together with the carbon atom to which they are attached, can form a cyclopropyl; or R⁵ and R⁶ together form an oxo.

In another preferred embodiment, in general formula (1), ring A is phenyl or (5- to 6-membered) heteroaryl, and ring A is preferably phenyl or (5- to 6-membered) heteroaryl containing 1 or 2 atoms independently selected from N, O, and S.

In another preferred embodiment, in general formula (1), ring A is

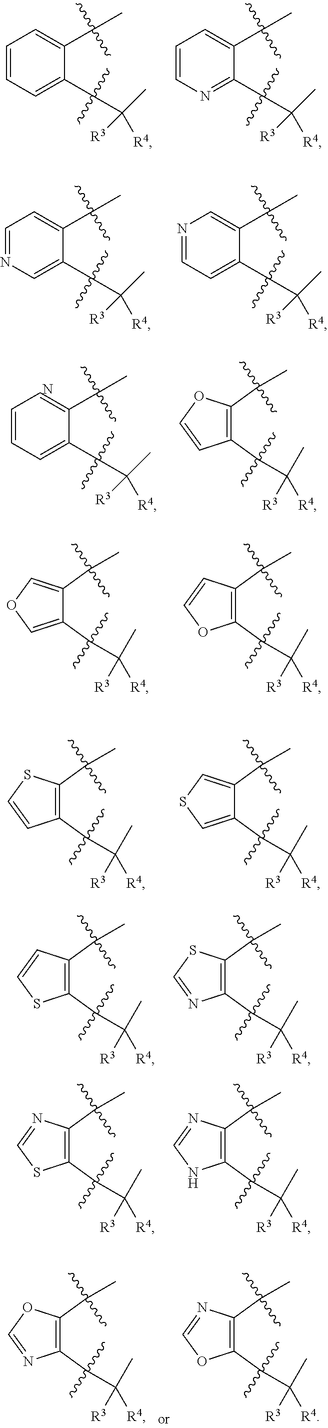

In another preferred embodiment, in general formula (1), each R¹ is independently and optionally: —H, —F, —Cl, —Br, —I, —OH, —NO₂, —N(CH₃)₂, —NH₂, —CH₂OCH₃, —CH₂N(CH₃)₂, —CN, (C1-C3) alkyl, (C1-C3) haloalkyl, (C1-C3) alkoxy, (C2-C4) alkenyl, (C2-C4) alkynyl, (C3-C6) cycloalkyl, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂, wherein the (C1-C3) alkyl, (C1-C3) haloalkyl, (C1-C3) alkoxy, (C2-C4) alkenyl, (C2-C4) alkynyl, or (C3-C6) cycloalkyl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN,

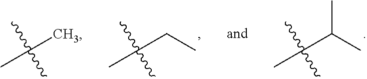

In another preferred embodiment, in general formula (1), each R¹ is independently: —H, —F, —Cl, —Br, —I, —OH, —NO₂, —N(CH₃)₂, —NH₂, —CH₂OCH₃, —CH₂N(CH₃)₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, —S(O)₂N(CH₃)₂,

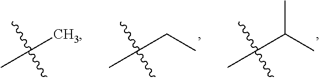

—CF₃, —CH₂CF₃,

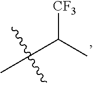 [SMILES: *C(C)C(F)(F)F]

—OCH₃, —OCH₂CH₃, —OCH(CH₃)₂,

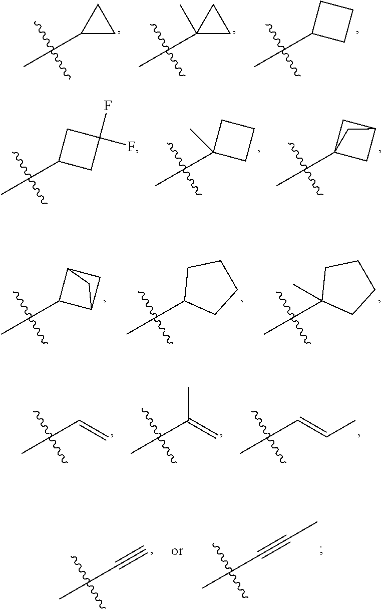

R¹ is preferably —H, —F, —Cl, —CN,

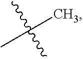

—CF₃, —OCH₃,

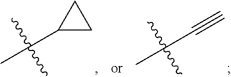

R¹ is more preferably —H, —F, —Cl, —CN,

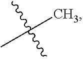

—CF₃, —OCH₃, or

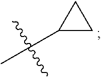 [SMILES: *C1CC1]

R¹ is more preferably —H, —F, —Cl, —CN, or

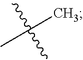

n is preferably 1; n is preferably 2; n is preferably 3.

In another preferred embodiment, in general formula (1), ring B is (C5-C6) cycloalkyl or (5- to 6-membered) heterocycloalkyl, preferably partially unsaturated (C5-C6) cycloalkyl or (5- to 6-membered) partially unsaturated heterocycloalkyl containing one atom independently selected from N, O, and S.

In another preferred embodiment, in general formula (1), the structural unit

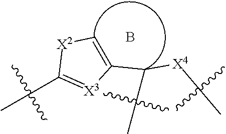

is:

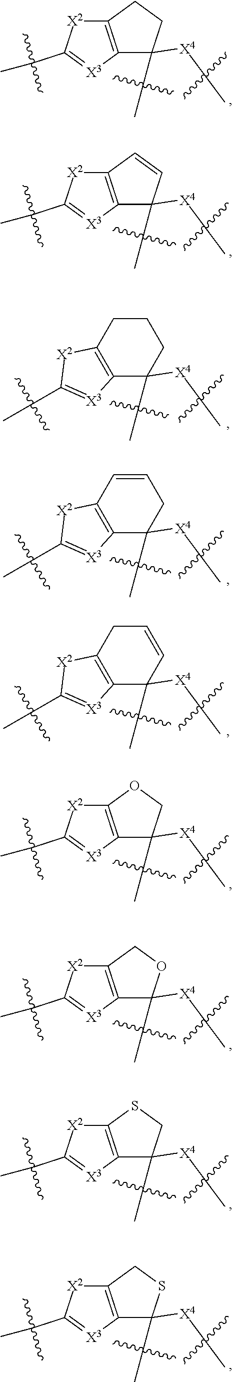

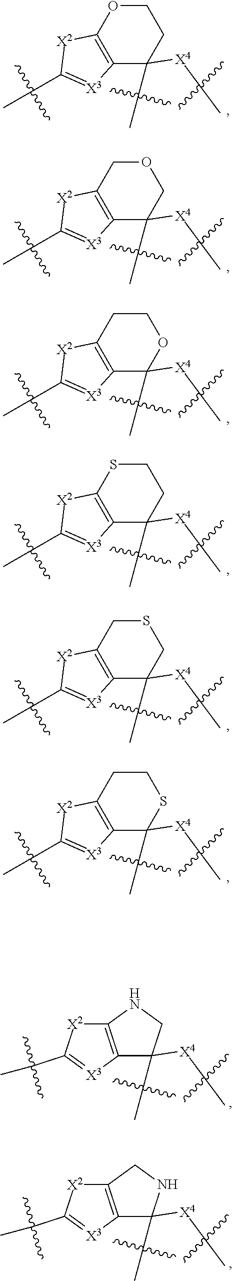

In another preferred embodiment, in general formula (1), the structural unit

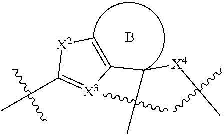

is:

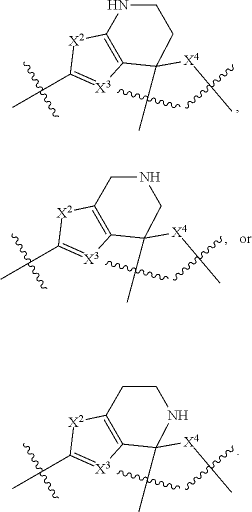

In another preferred embodiment, in general formula (1), each R² is independently and optionally: —H, —F, —Cl, —Br, —I, —OH, —N(CH₃)₂, —NH₂, —CN, (C1-C3) alkyl, (C1-C3) haloalkyl, (C1-C3) alkoxy, (C2-C4) alkenyl, (C2-C4) alkynyl, or (C3-C5) cycloalkyl; or two R² on a same carbon atom, together with the carbon atom to which they are attached, can form (4- to 5-membered) heterocycloalkyl or (C3-C5) cycloalkyl, wherein the (4- to 5-membered) heterocycloalkyl or (C3-C5) cycloalkyl may be optionally substituted with 1, 2, 3, or 4 of the C3 following groups: —H, —F,

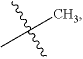

and —OCH₃; or two R² on a same carbon atom together form an oxo.

In another preferred embodiment, in general formula (1), each R² is independently and optionally: —H, —F, —Cl, —Br, —I, —OH, —N(CH₃)₂, —NH₂, —CN,

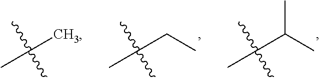

CF₃, CH₂—CF₃,

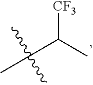 [SMILES: *C(C)C(F)(F)F]

—OCH₃, —OCH₂CH₃, —OCH(CH₃)₂,

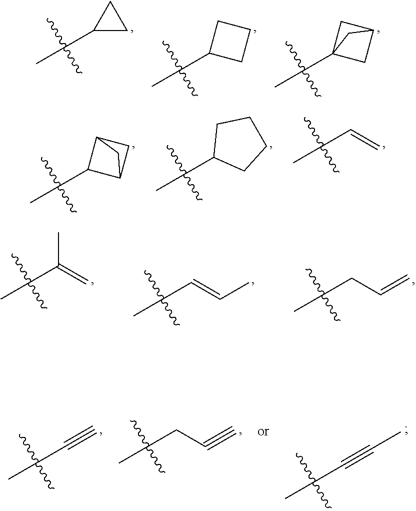

R² is preferably —H, —F, —Cl, —CN,

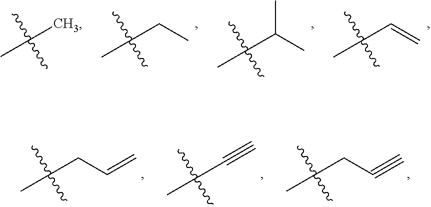

—CF₃, —CH₂CF₃, —OCH₃, or

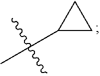 [SMILES: *C1CC1]

R¹ is more preferably —H, —F, —Cl,

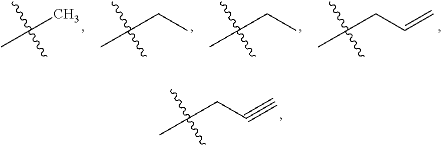

—CF₃, —CH₂CF₃, or

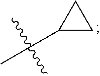 [SMILES: *C1CC1]

R¹ is more preferably —H, —F,

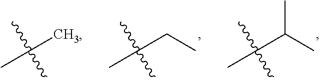

CH₂CF₃,

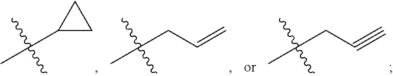

m is preferably 1; m is preferably 2.

In another preferred embodiment, in general formula (1), the structural unit

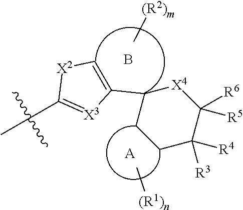

is

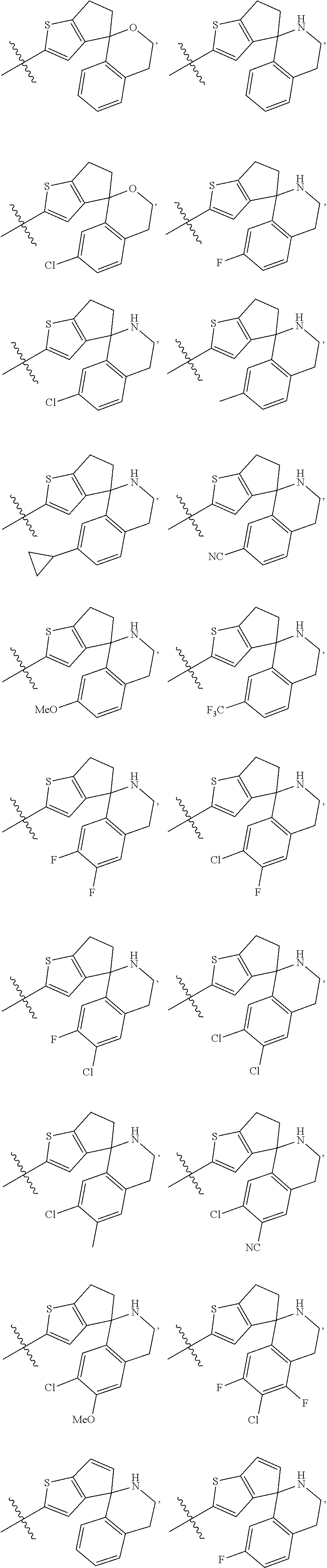

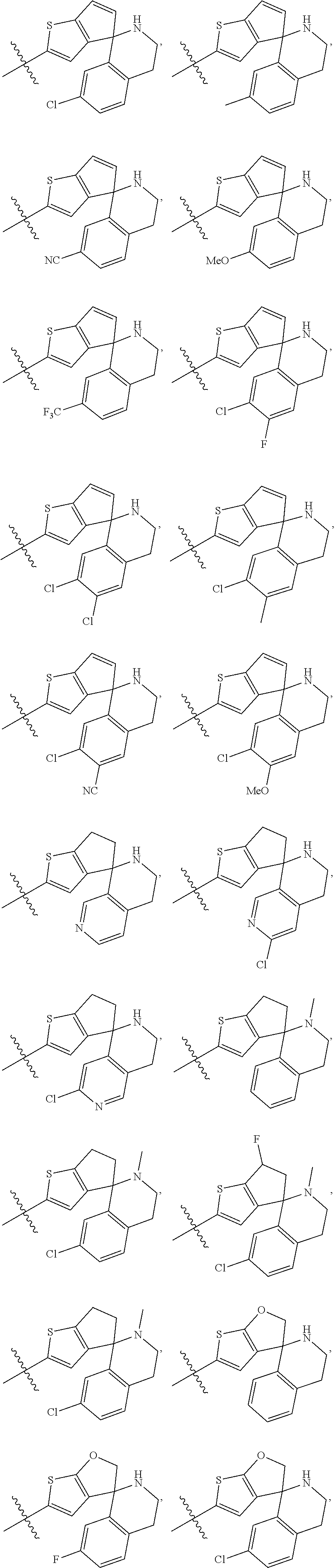

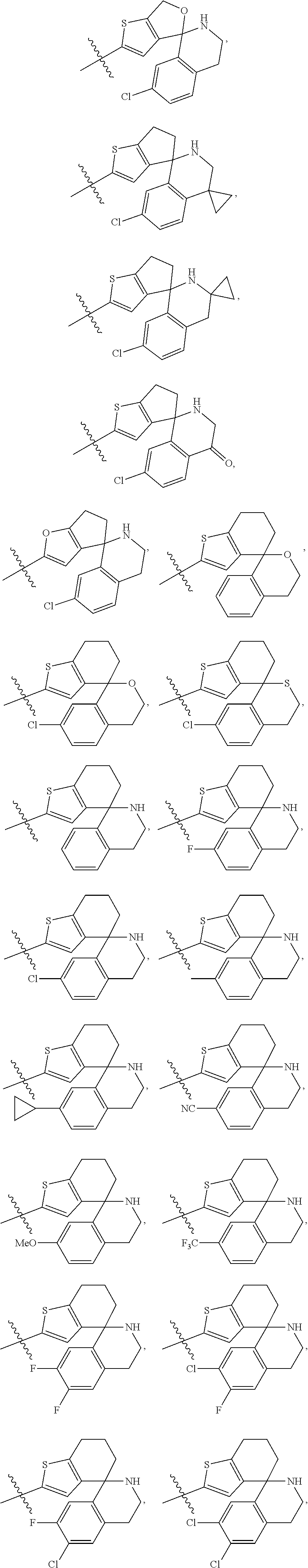

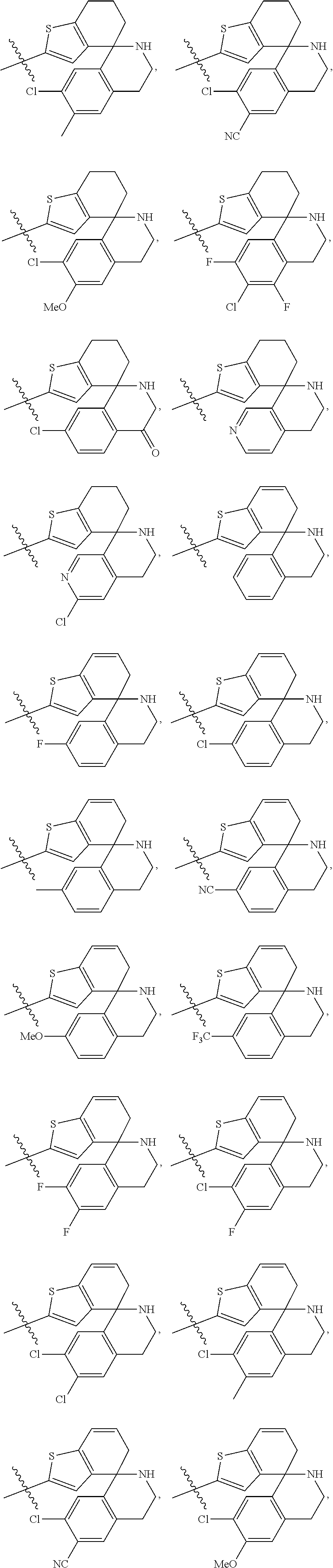

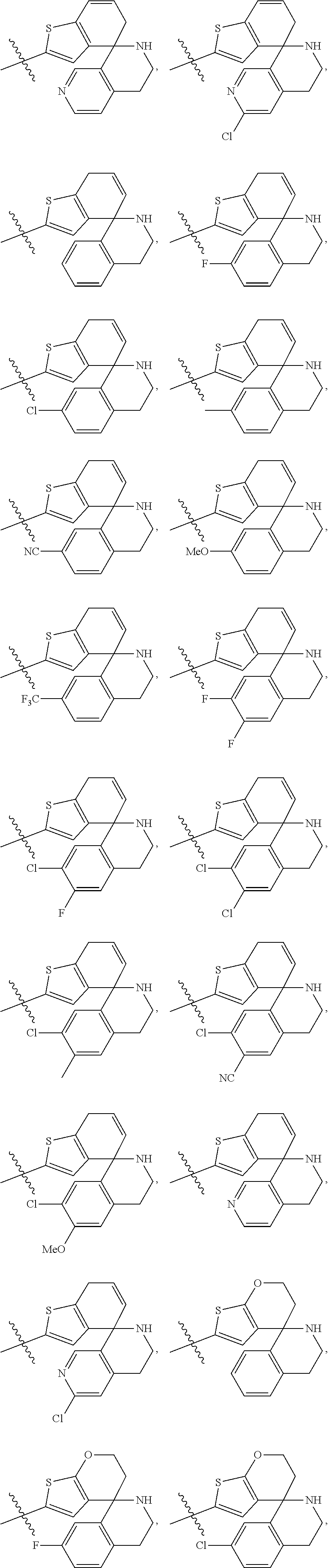

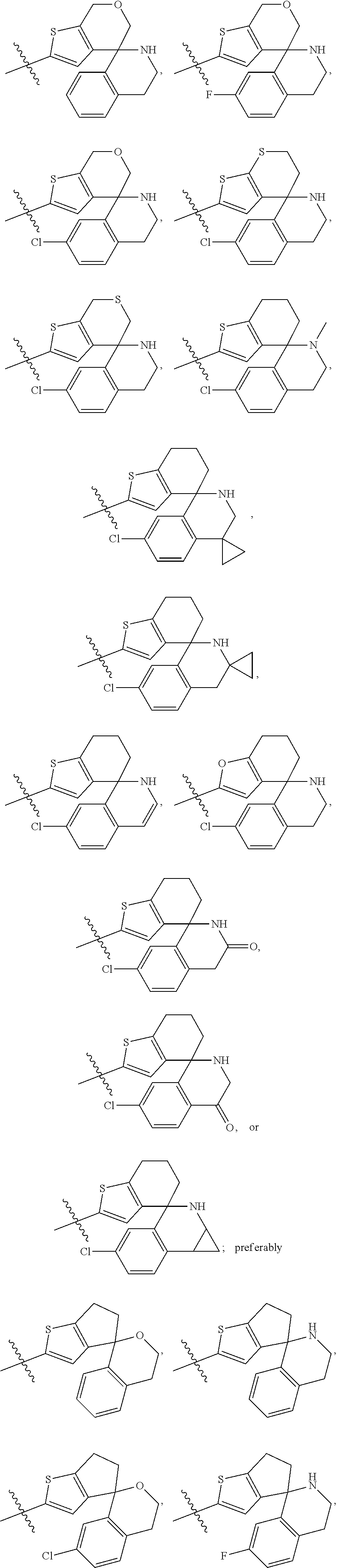

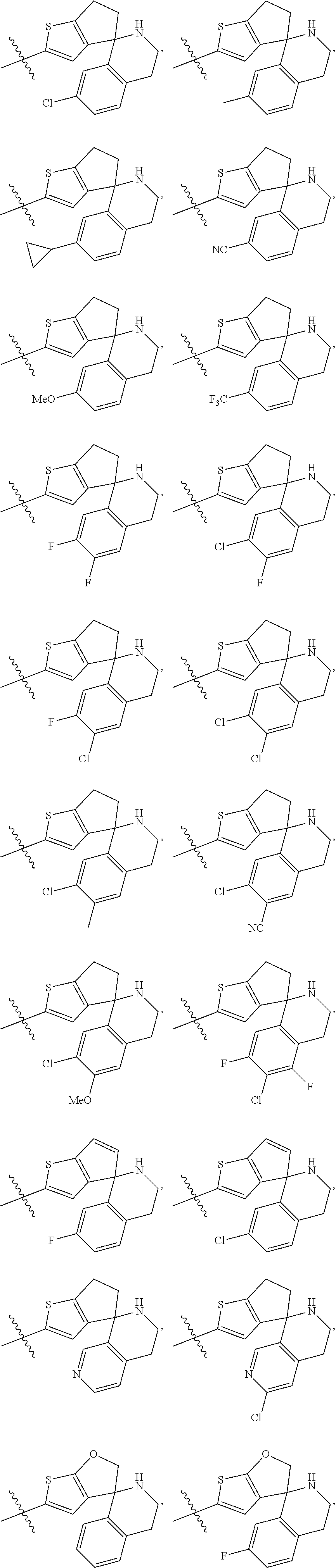

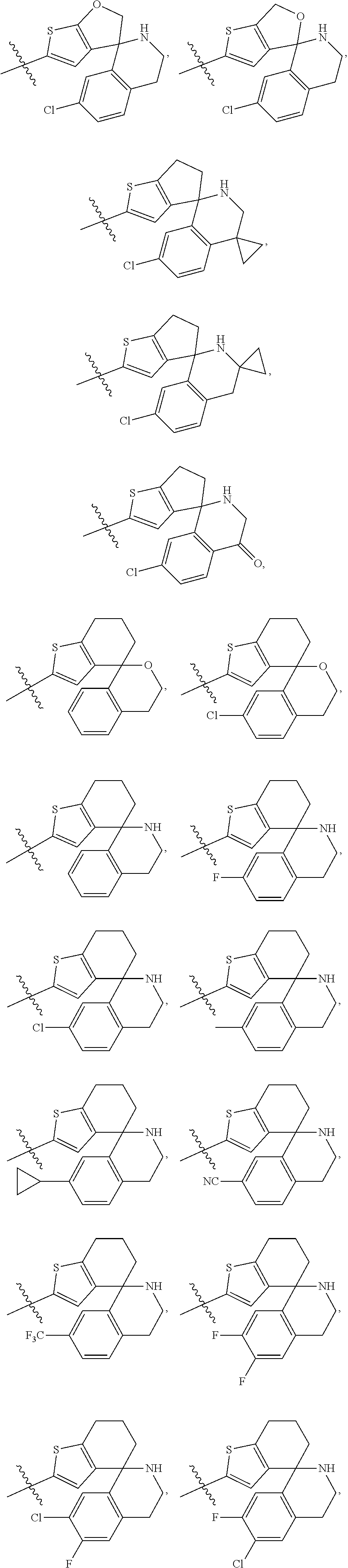

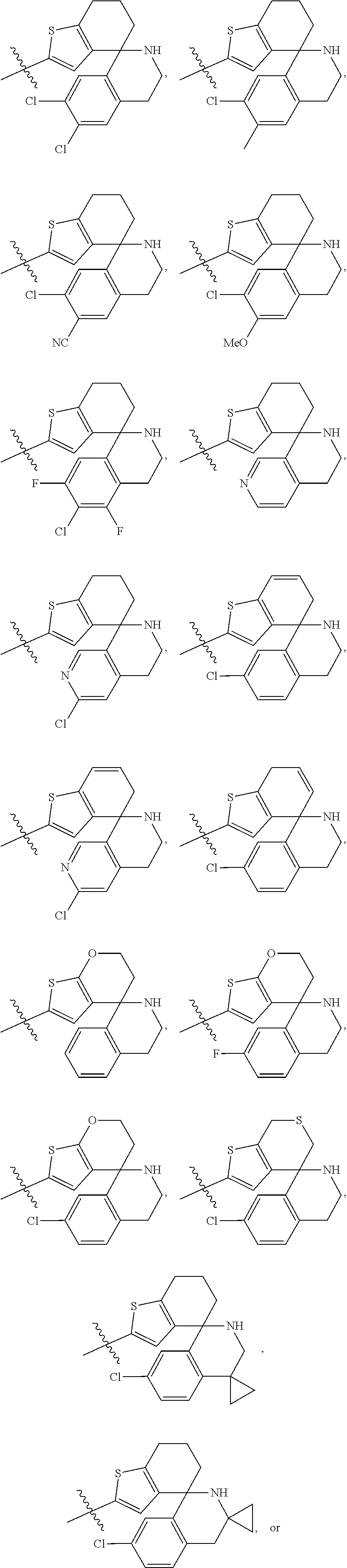

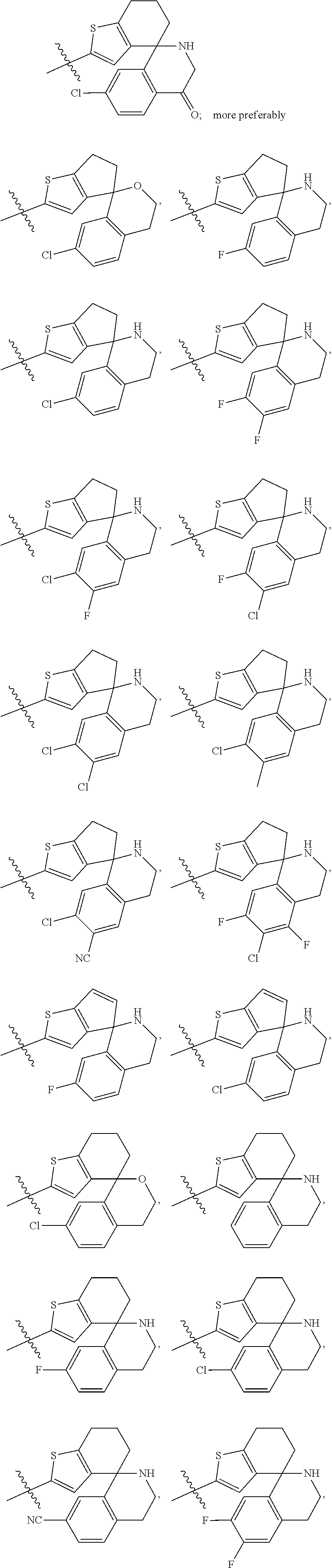

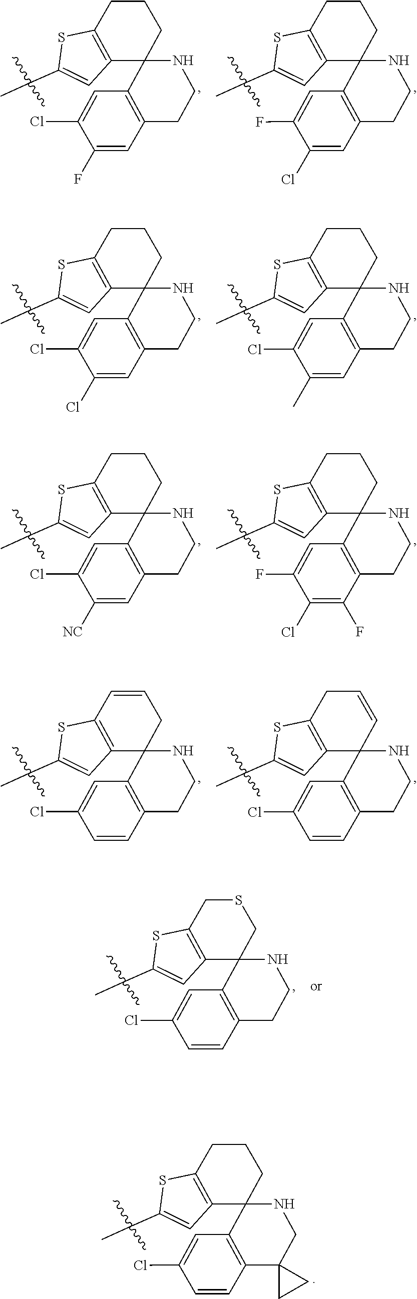

In another preferred embodiment, in general formula (1), the structural unit

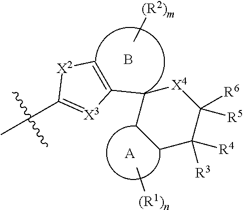

is

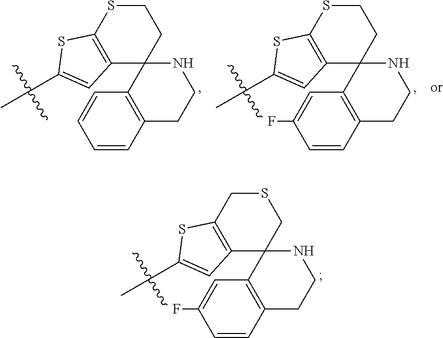 [SMILES: *c1cc2c(s1)SCCC21NCCc2ccccc21 ; *c1cc2c(s1)SCCC21NCCc2ccc(F)cc21 ; *c1cc2c(s1)CSCC21NCCc2ccc(F)cc21]

preferably

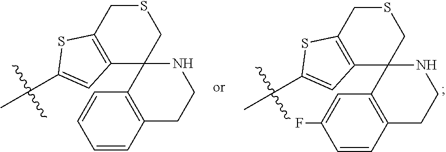 [SMILES: *c1cc2c(s1)CSCC21NCCc2ccccc21 ; *c1cc2c(s1)CSCC21NCCc2ccc(F)cc21]

and more preferably

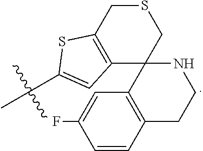 [SMILES: *c1cc2c(s1)CSCC21NCCc2ccc(F)cc21]

In another preferred embodiment, in general formula (1), the structural unit

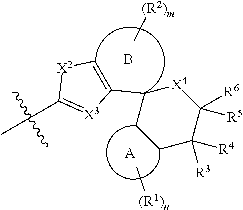

is

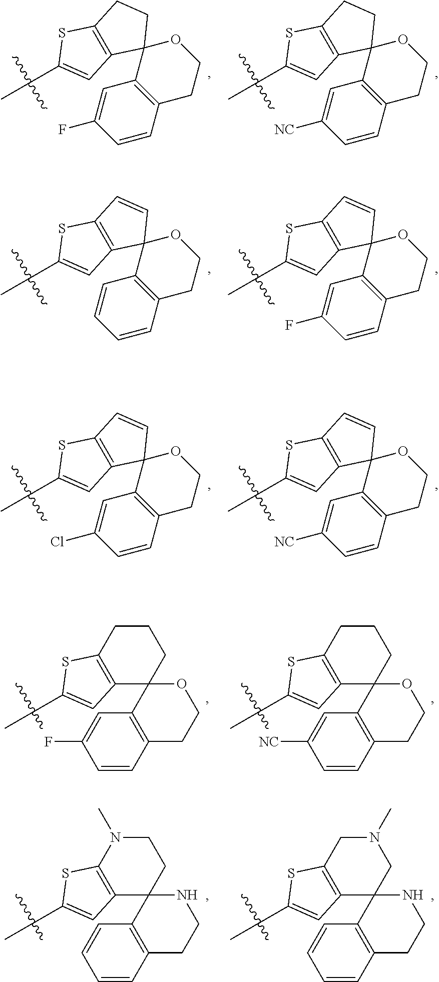

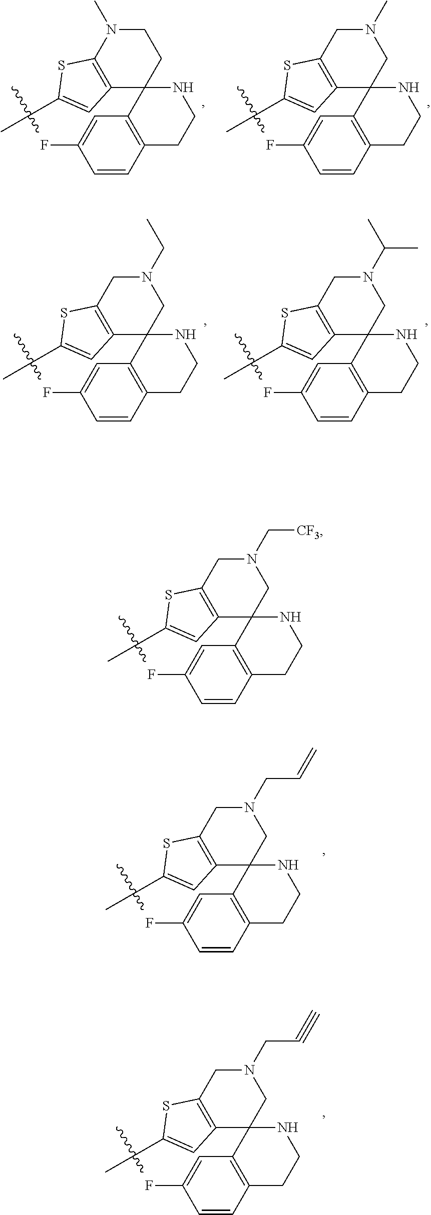

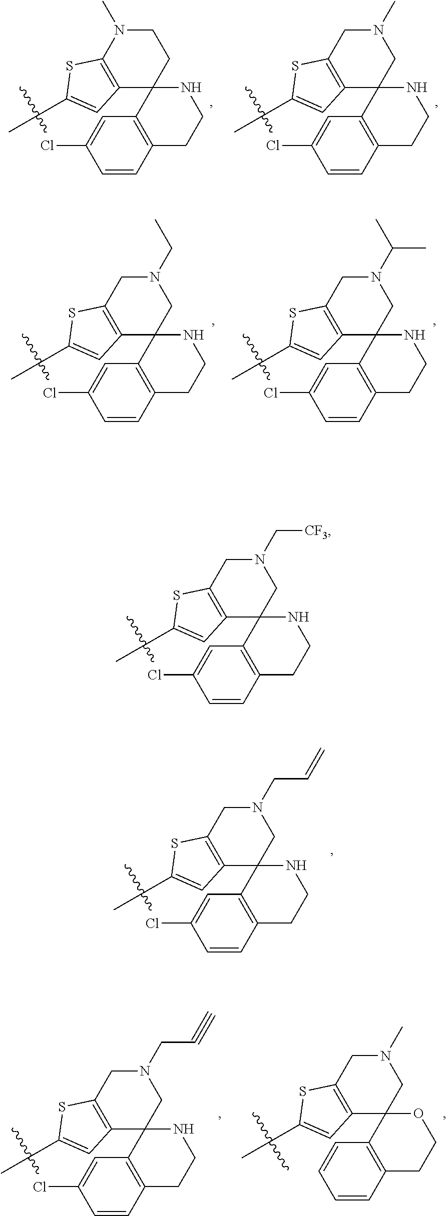

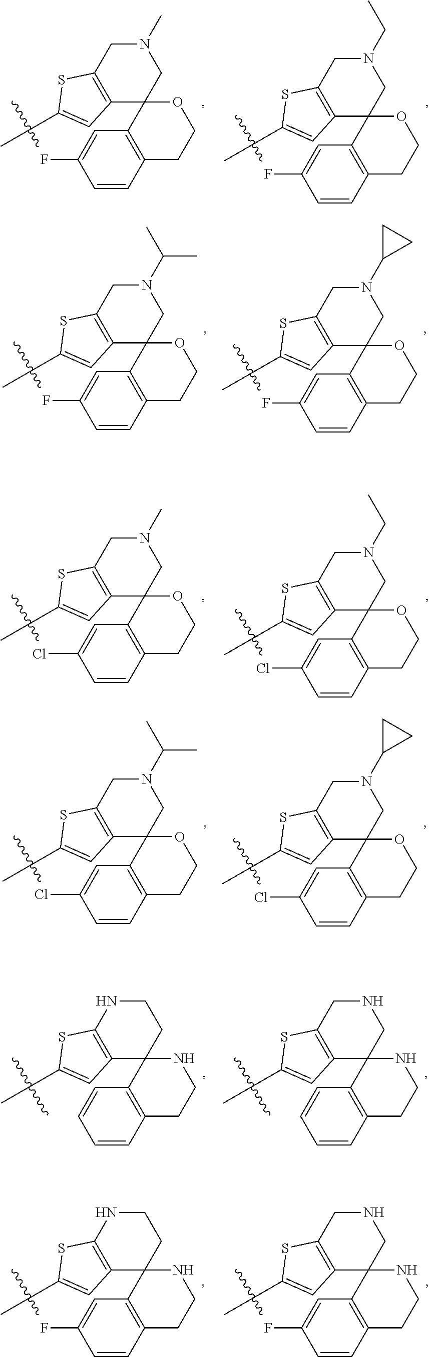

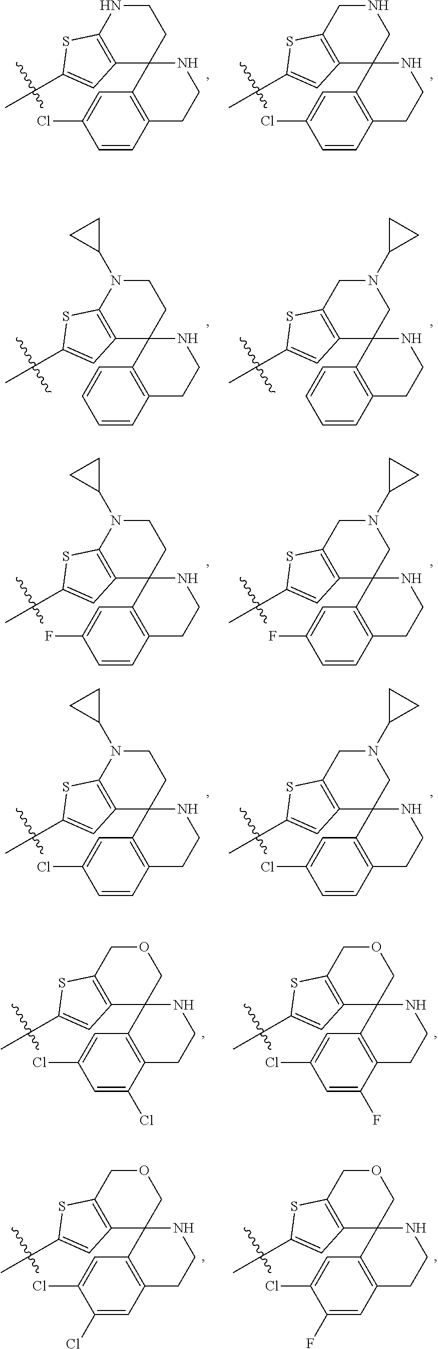

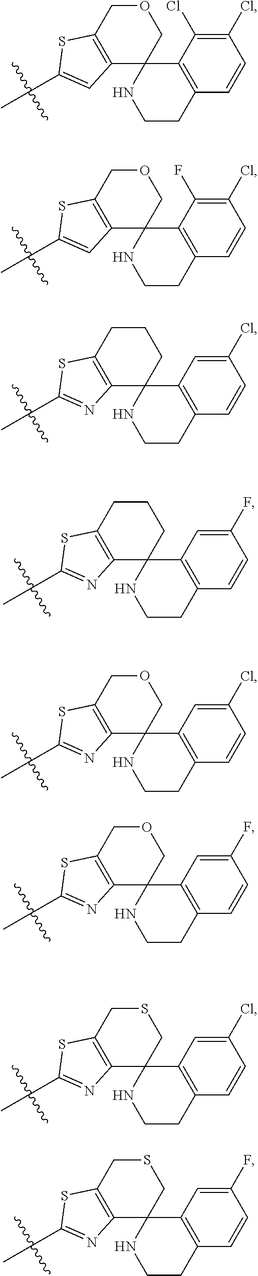

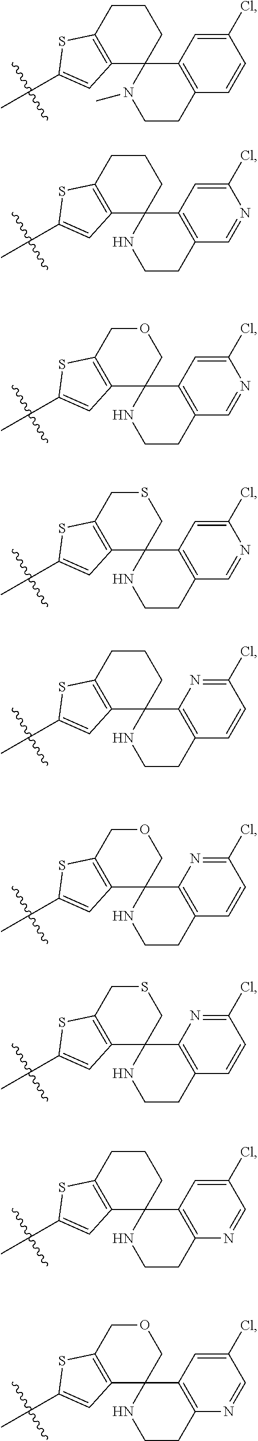

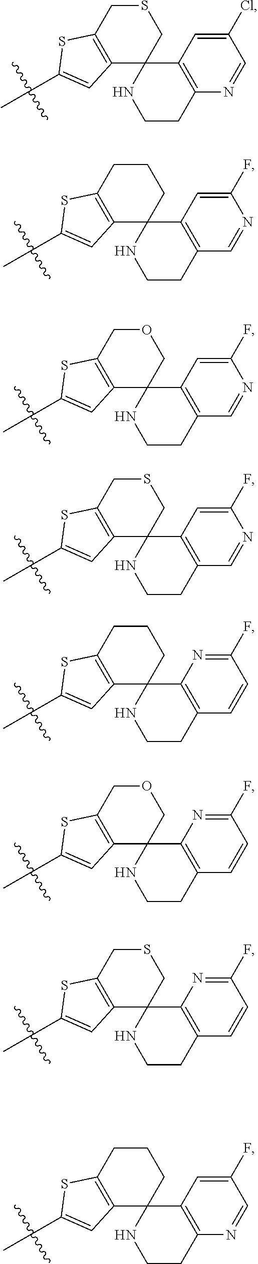

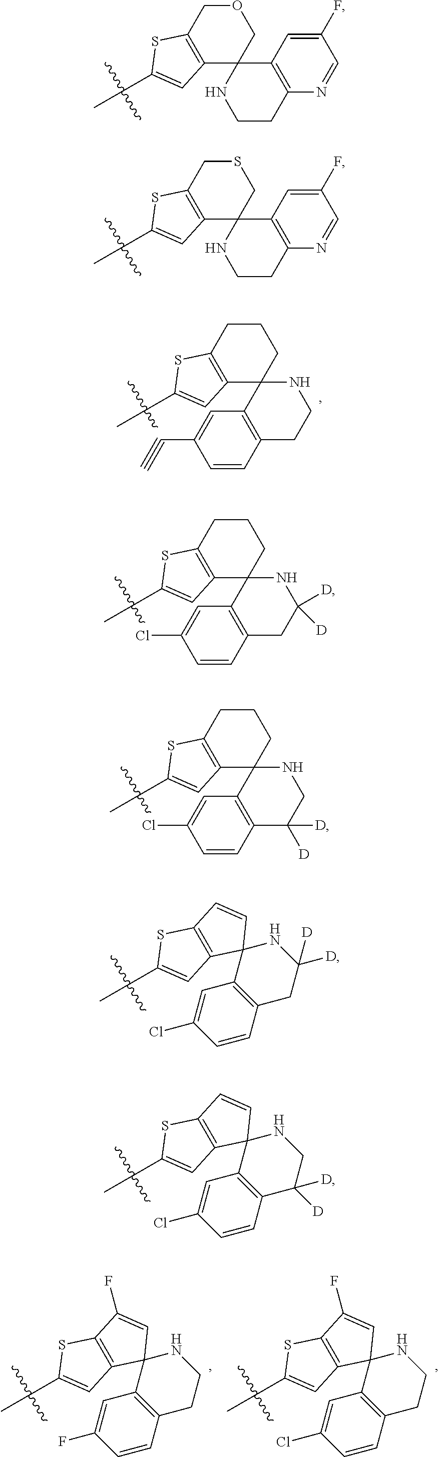

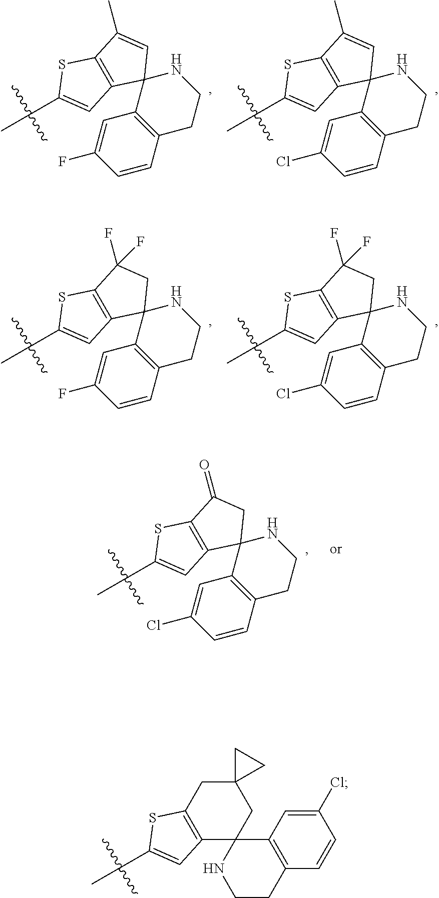 [SMILES: *c1cc2c(s1)C(C)=CC21NCCc2ccc(F)cc21 ; *c1cc2c(s1)C(C)=CC21NCCc2ccc(Cl)cc21 ; *c1cc2c(s1)C(F)(F)CC21NCCc2ccc(F)cc21 ; *c1cc2c(s1)C(F)(F)CC21NCCc2ccc(Cl)cc21 ; *c1cc2c(s1)C(=O)CC21NCCc2ccc(Cl)cc21 ; *c1cc2c(s1)CC1(CC1)CC21NCCc2ccc(Cl)cc21]

preferably

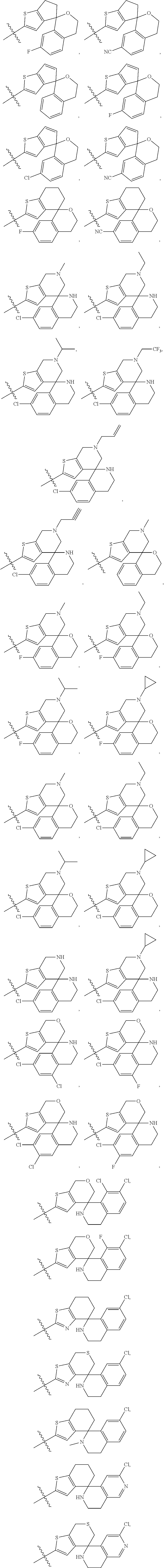

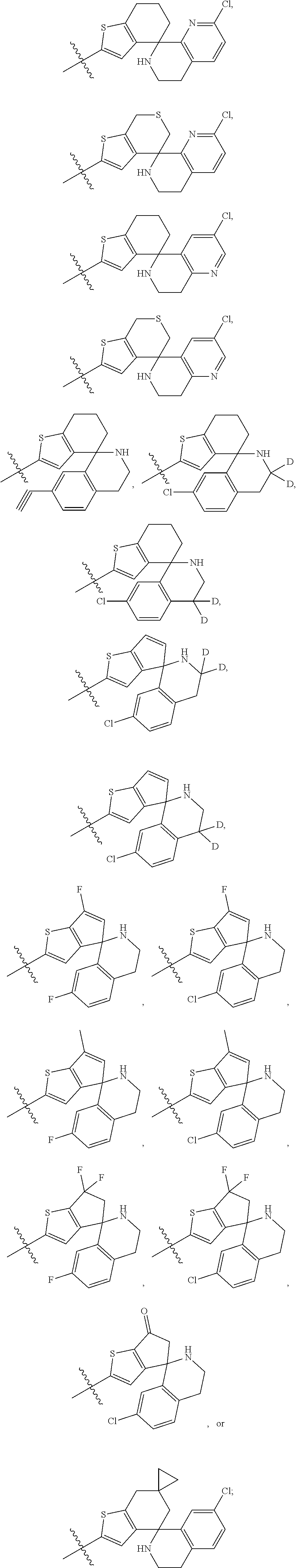

more preferably

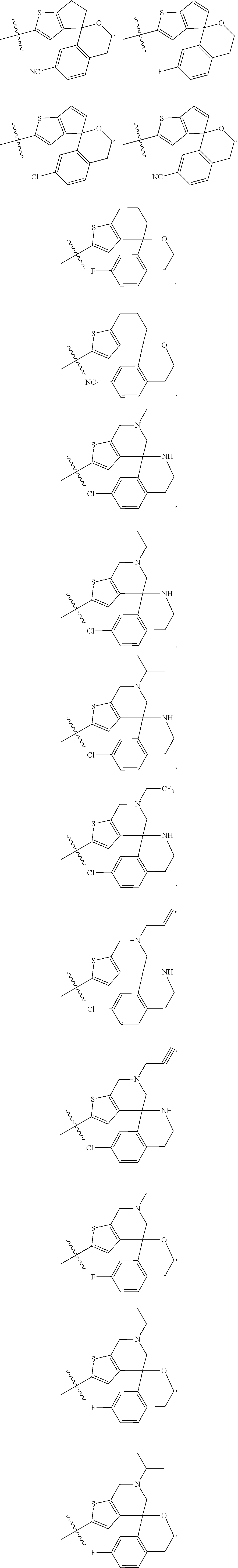

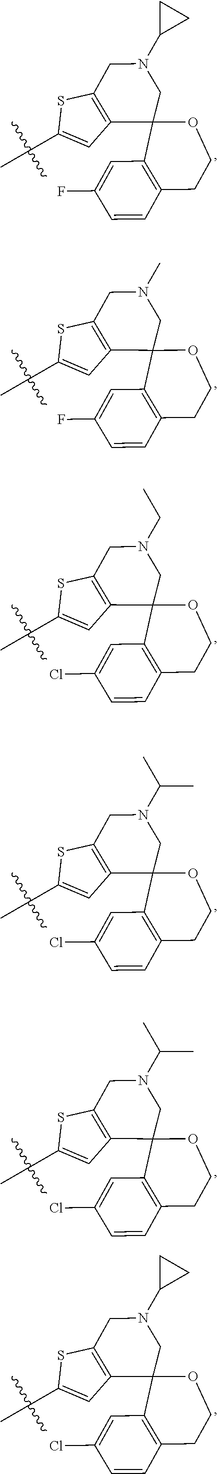

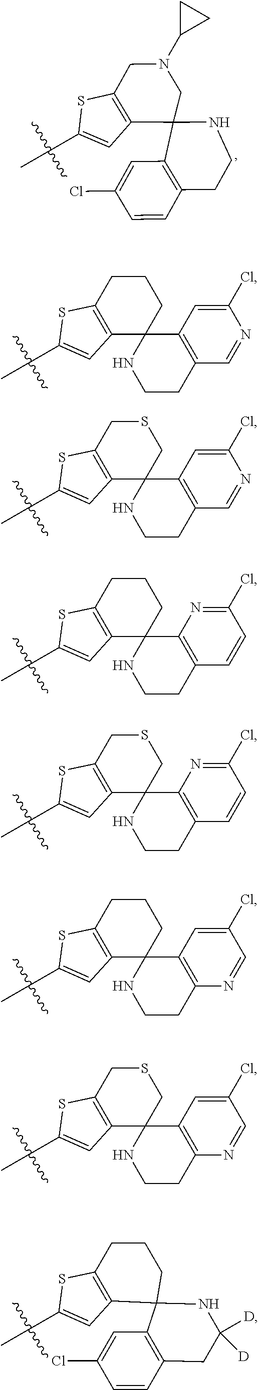

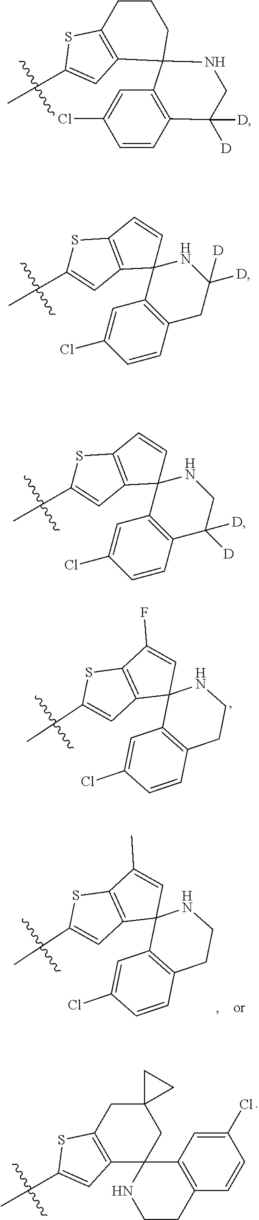

In another preferred embodiment, in general formula (1), general formula (1) has a structure as shown in general formula (1a)

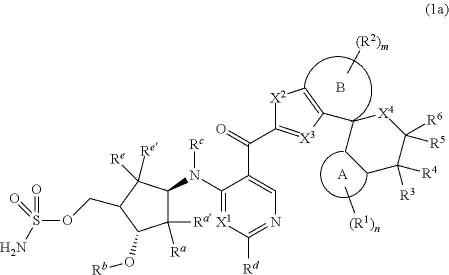

wherein ring A, ring B, R¹, R², R³, R⁴, R⁵, R⁶, R^{a}, R^{a′}, R^{b}, R^{c}, R^{d}, R^{e}, R^{e′}, X¹, X², X³, X⁴, m, and n are as previously defined and exemplified in the detailed examples.

In another preferred embodiment, in general formula (1), general formula (1) has a structure as shown in general formula (1b)

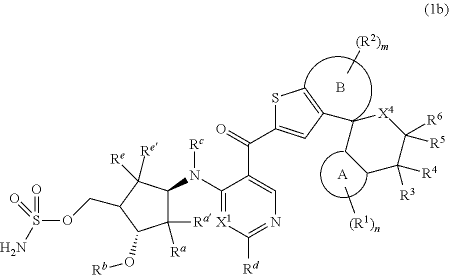

wherein ring A, ring B, R¹, R², R³, R⁴, R⁵, R⁶, R^{a}, R^{a′}, R^{b}, R^{c}, R^{d}, R^{e}, R^{e′}, X¹, X⁴, m, and n are as previously defined and exemplified in the detailed examples.

In another preferred embodiment, in general formula (1), general formula (1) has a structure as shown in general formula (1c)

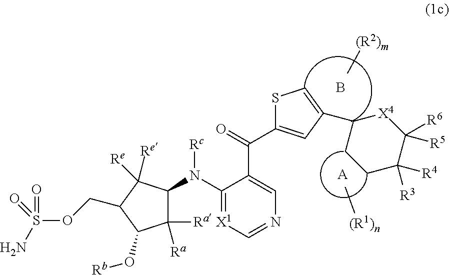

wherein ring A, ring B, R¹, R², R³, R⁴, R⁵, R⁶, R^{a}, R^{a′}, R^{b}, R^{c}, R^{e}, R^{e′}, X¹, X⁴, m, and n are as previously defined and exemplified in the detailed examples.

In another preferred embodiment, in general formula (1), general formula (1) has a structure as shown in general formula (1d)

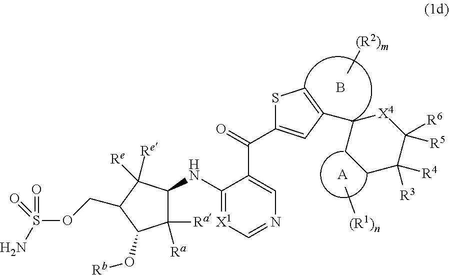

wherein ring A, ring B, R¹, R², R³, R⁴, R⁵, R⁶, R^{a}, R^{a′}, R^{b}, R^{e}, R^{e′}, X¹, X⁴, m, and n are as previously defined and exemplified in the detailed examples.

In another preferred embodiment, in general formula (1), general formula (1) has a structure as shown in general formula (1e)

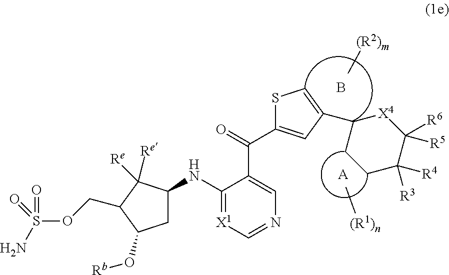

wherein ring A, ring B, R¹, R², R³, R⁴, R⁵, R⁶, R^{b}, R^{e}, R^{e′}, X¹, X⁴, m, and n are as previously defined and exemplified in the detailed examples.

In another preferred embodiment, in general formula (1), general formula (1) has a structure as shown in general formula (1g):

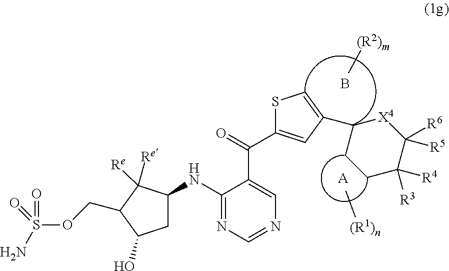

wherein ring A, ring B, R¹, R², R³, R⁴, R⁵, R⁶, R^{e}, R^{e′}, X⁴, m, and n are as previously defined and exemplified in the detailed examples.

In another preferred embodiment, in general formula (1), general formula (1) has a structure as shown in general formula (1h) or (1i)

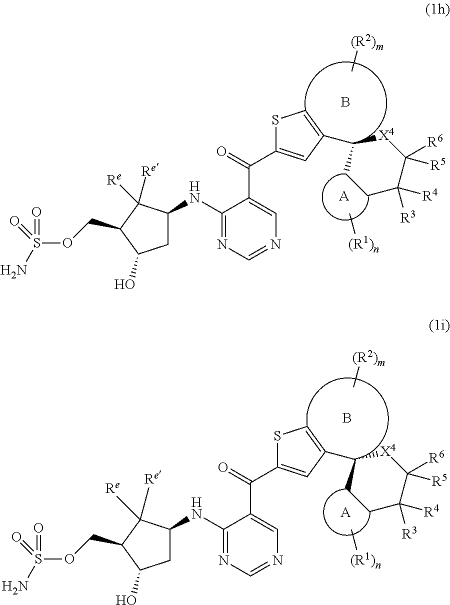

wherein ring A, ring B, R¹, R², R³, R⁴, R⁵, R⁶, R^{e}, R^{e′}, X⁴, m, and n are as previously defined and exemplified in the detailed examples.

In another preferred embodiment, in general formula (1), general formula (1) has a structure as shown in general formula (1j), (1k), (1l), or (1m)

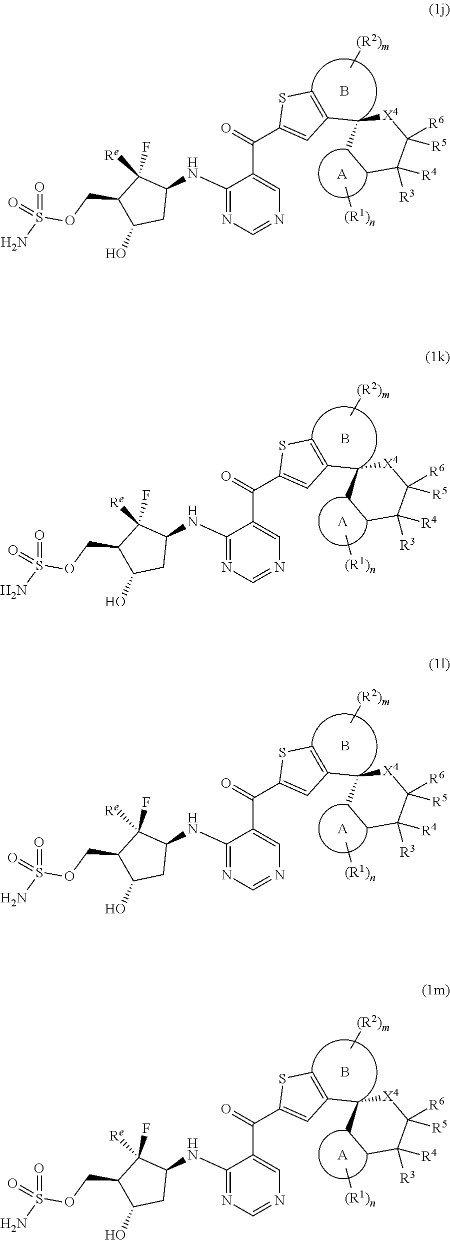

wherein ring A, ring B, R¹, R², R³, R⁴, R⁵, R⁶, R^{e}, X⁴, m, and n are as previously defined and exemplified in the detailed examples.

In another specific embodiment of the present disclosure, the compound of general formula (1) has one of the following structures:

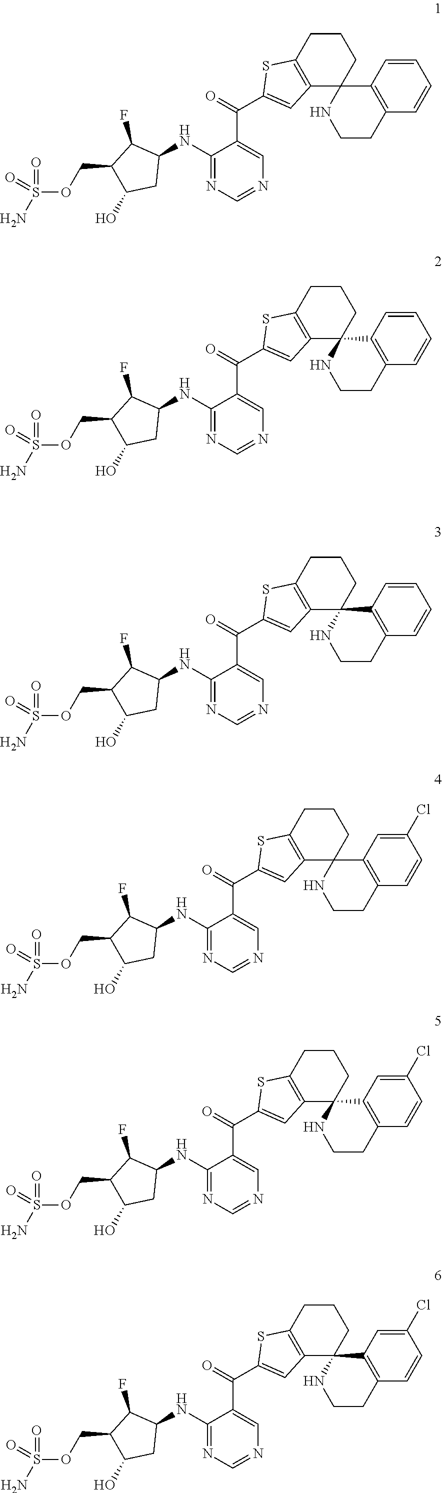

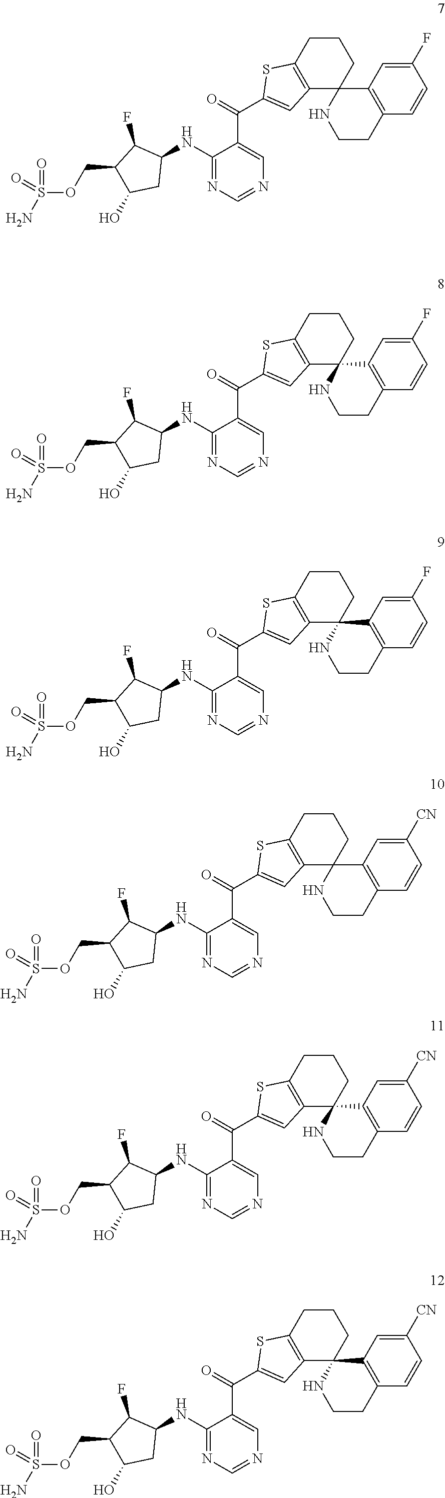

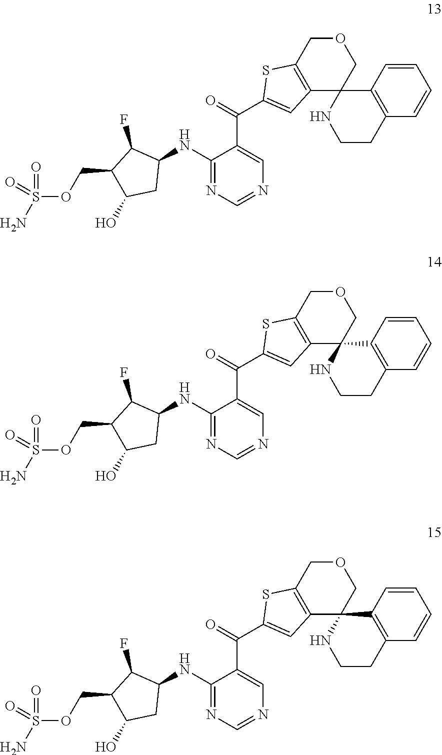

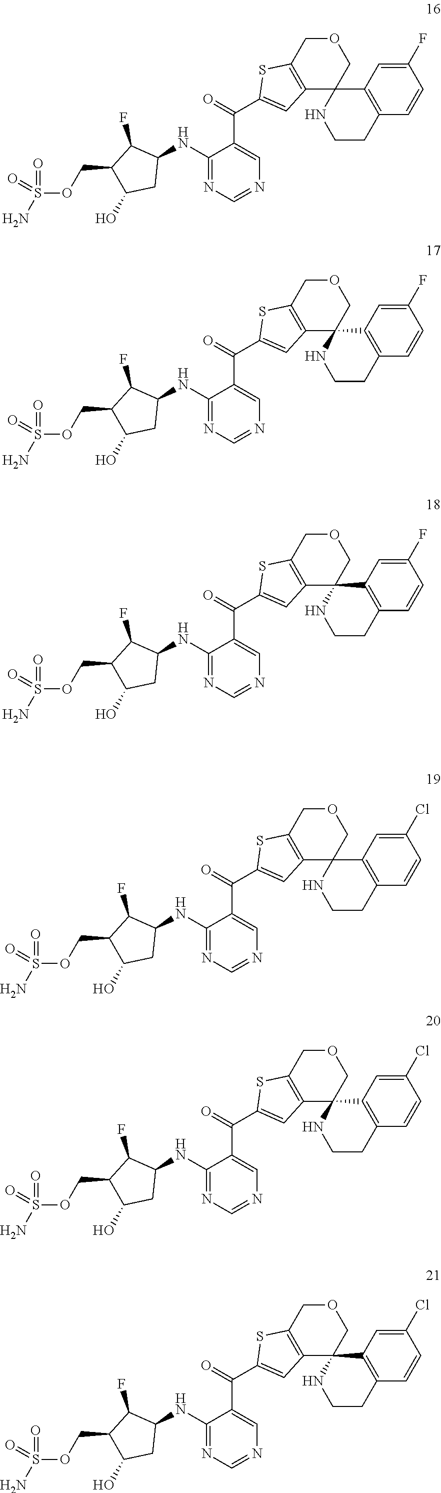

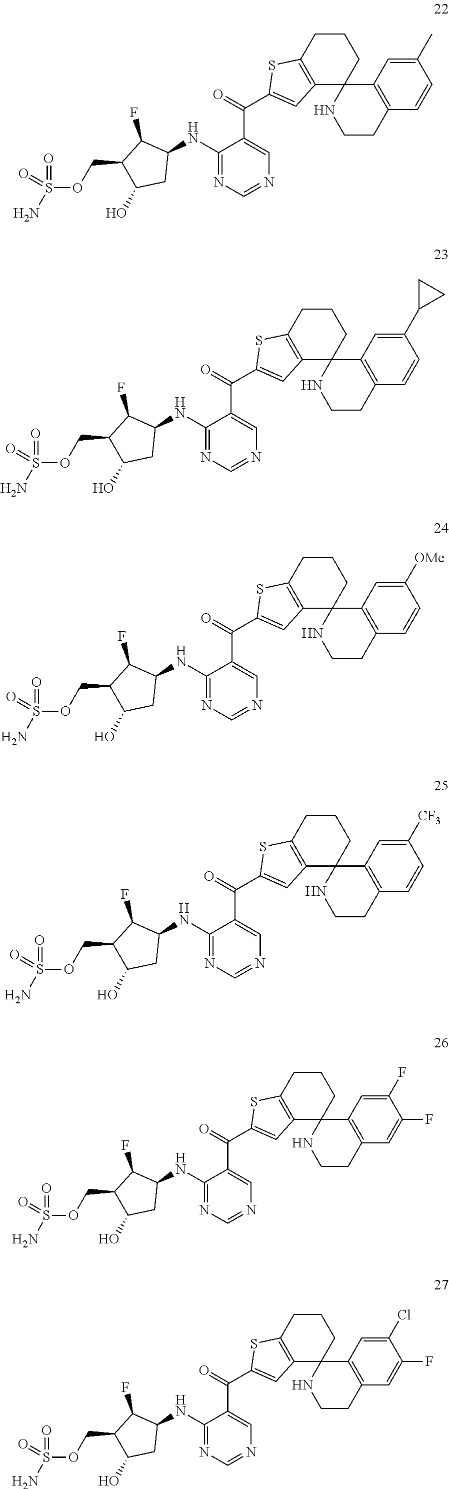

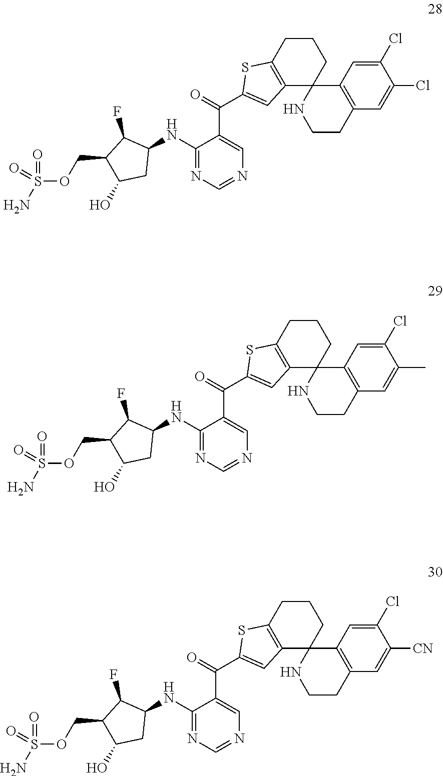

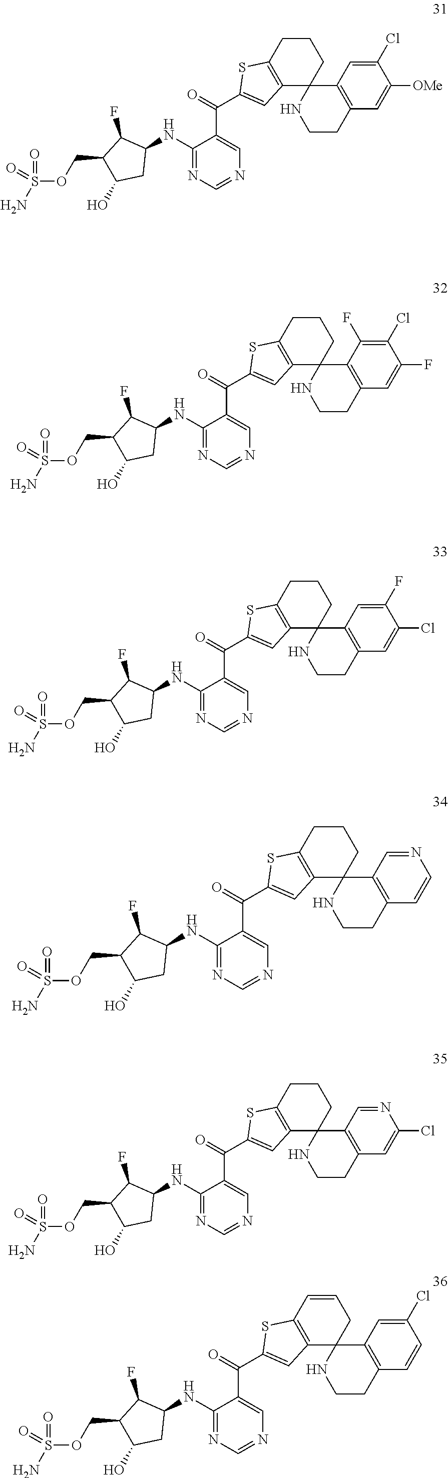

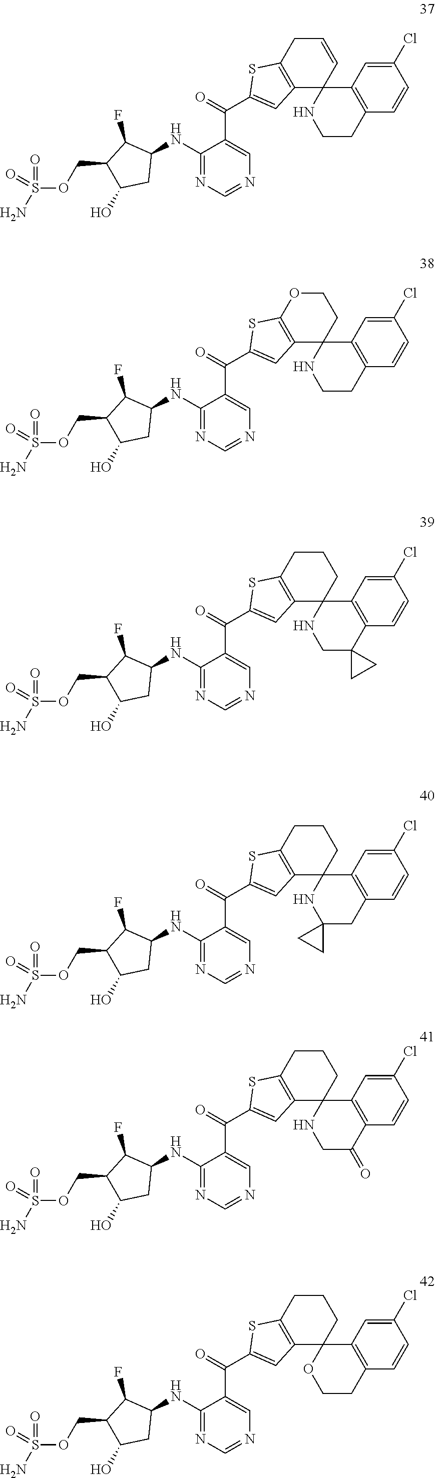

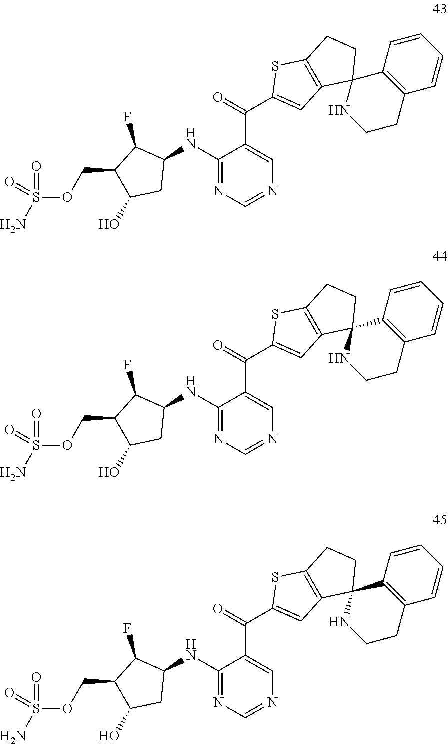

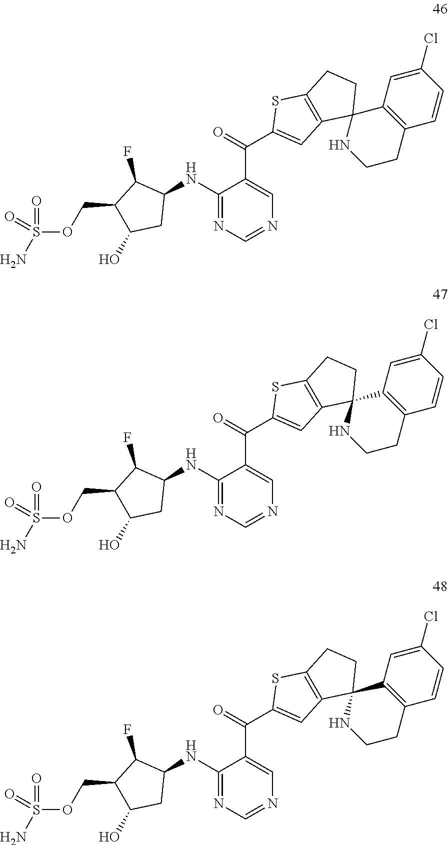

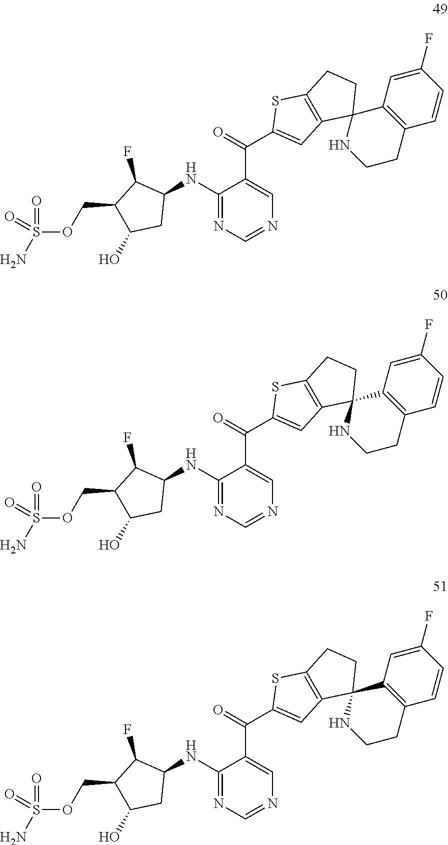

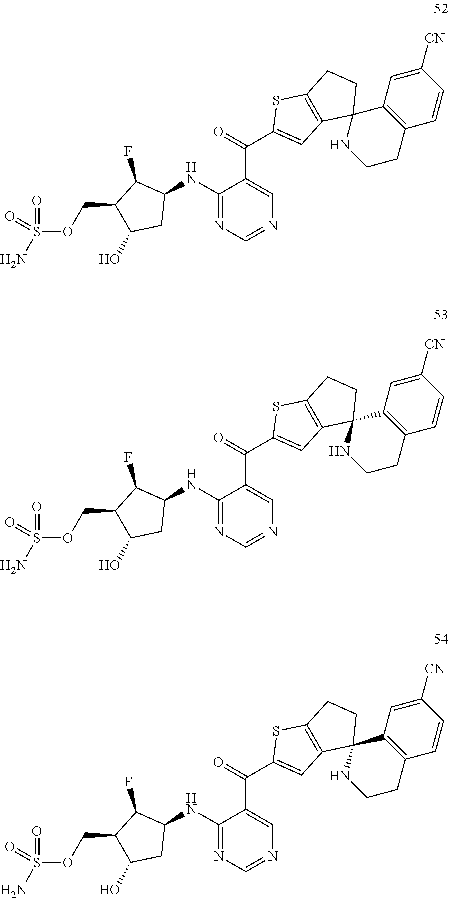

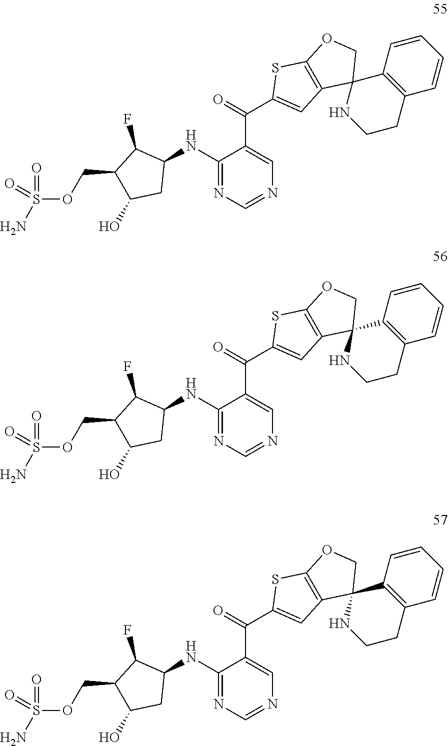

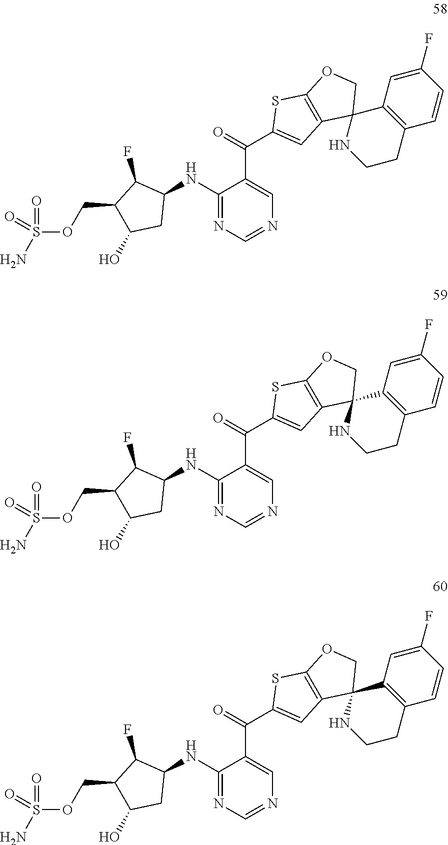

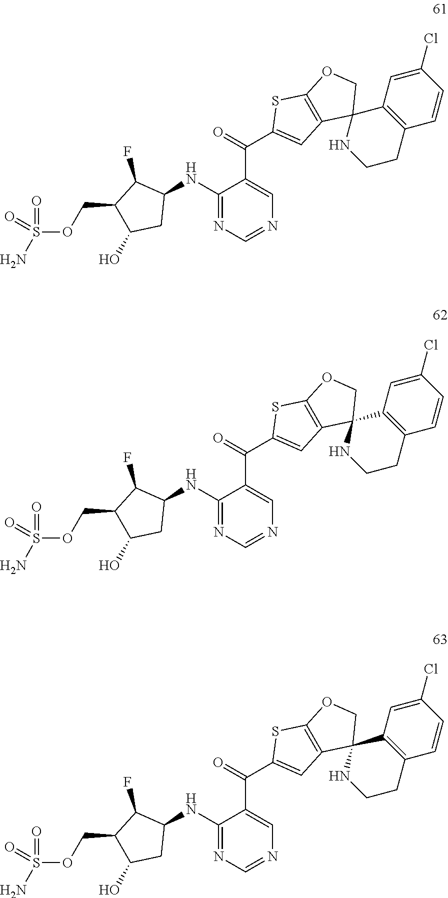

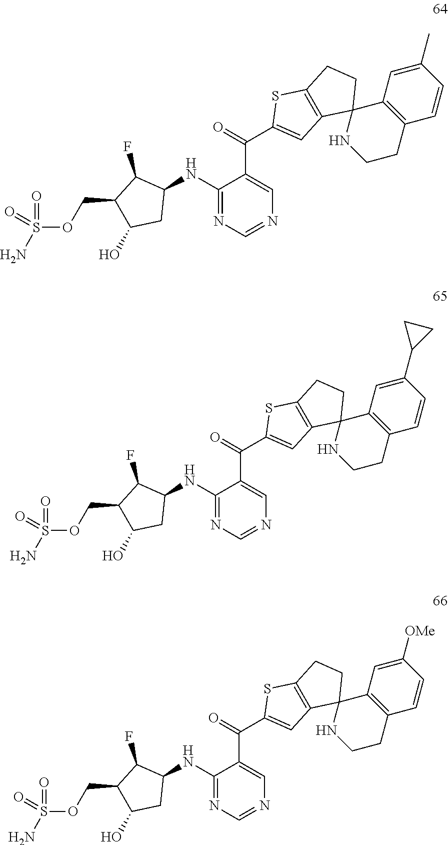

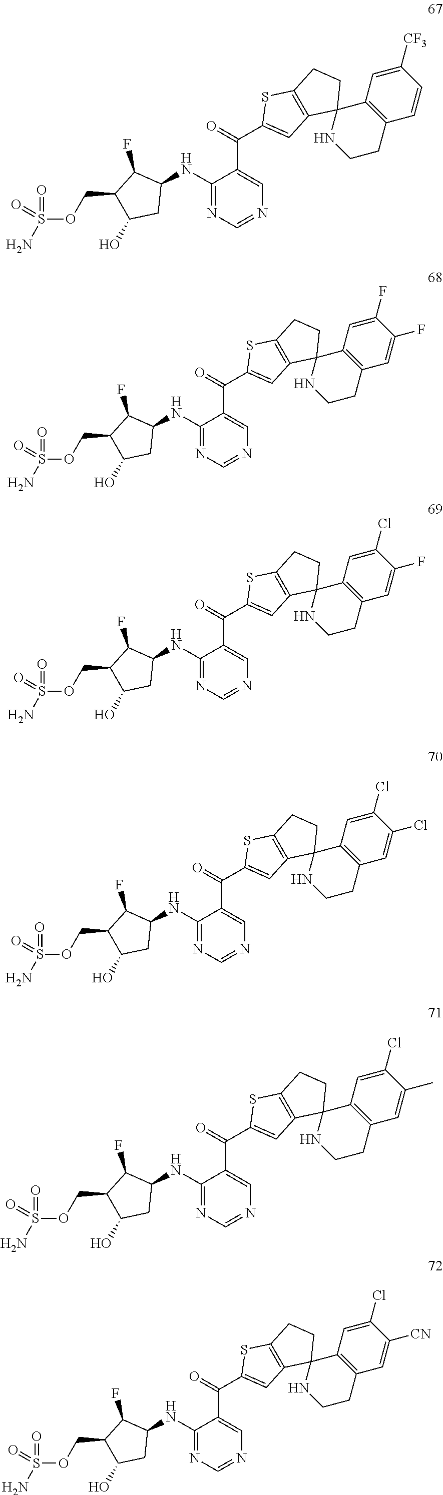

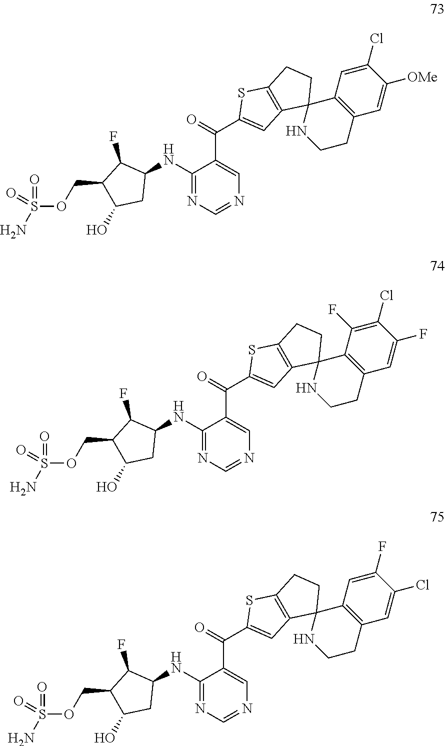

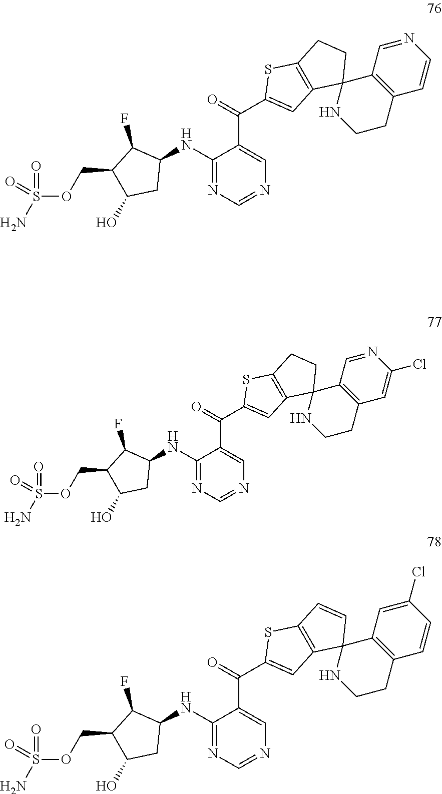

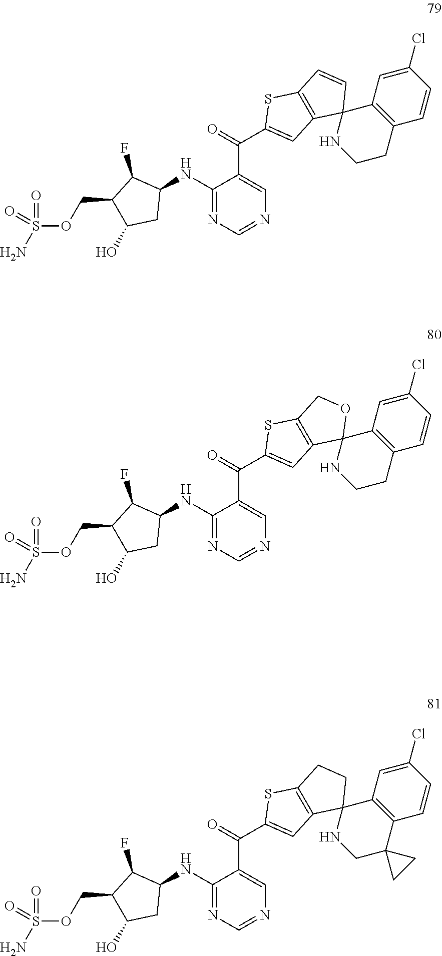

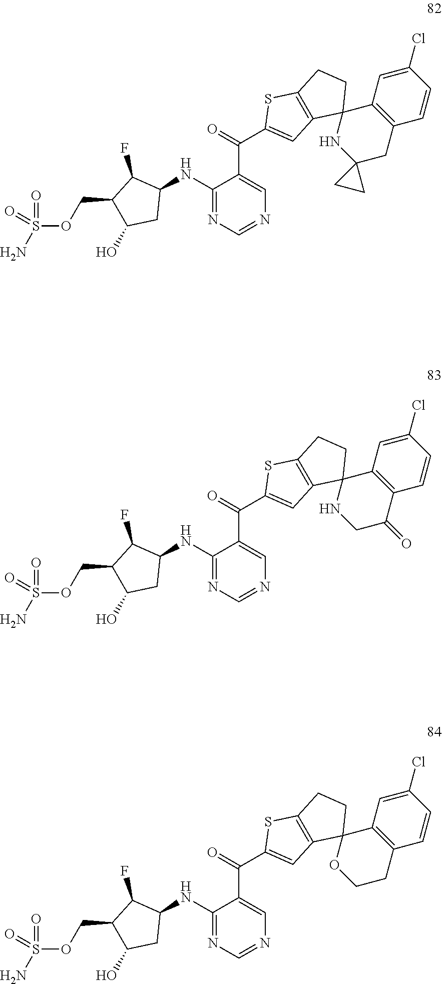

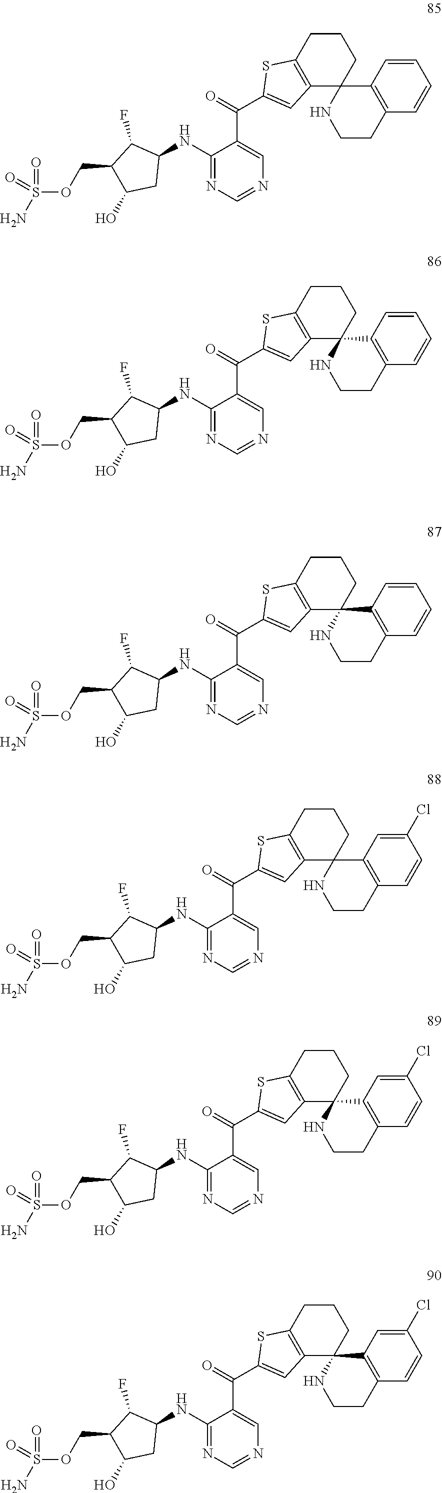

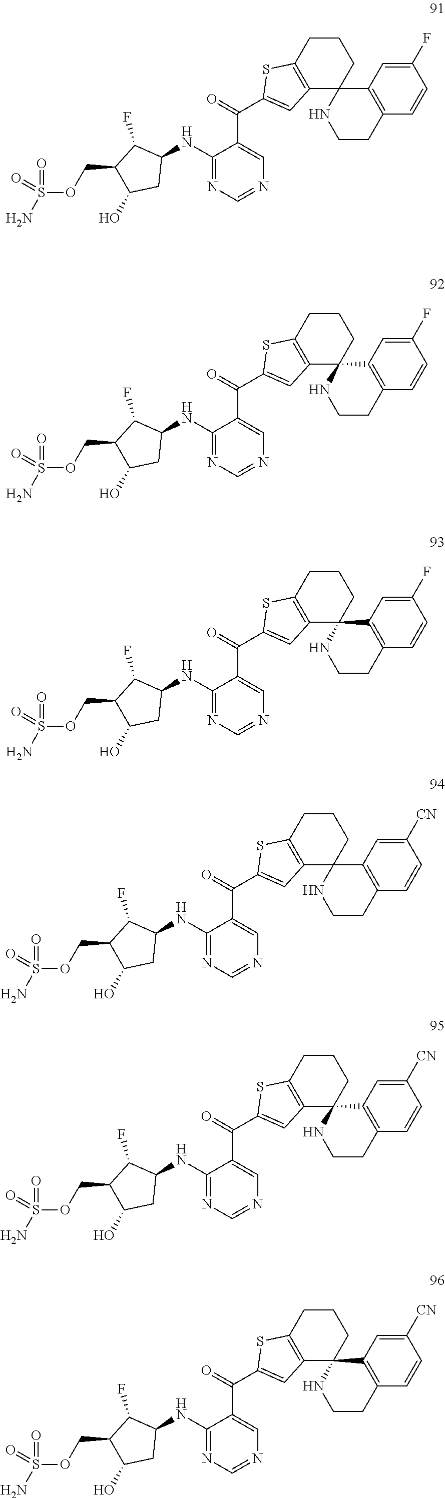

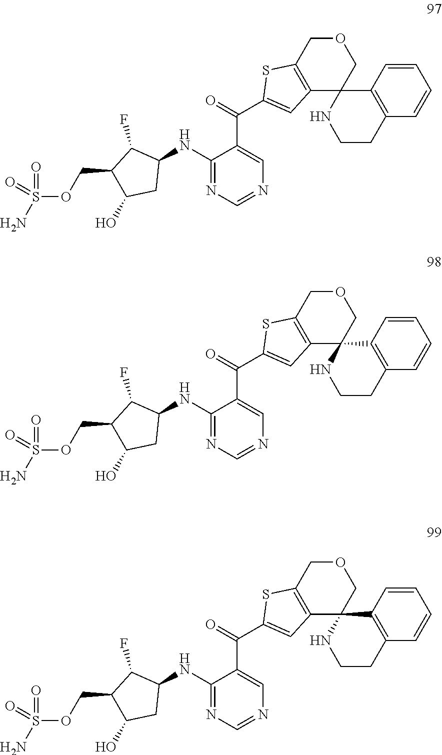

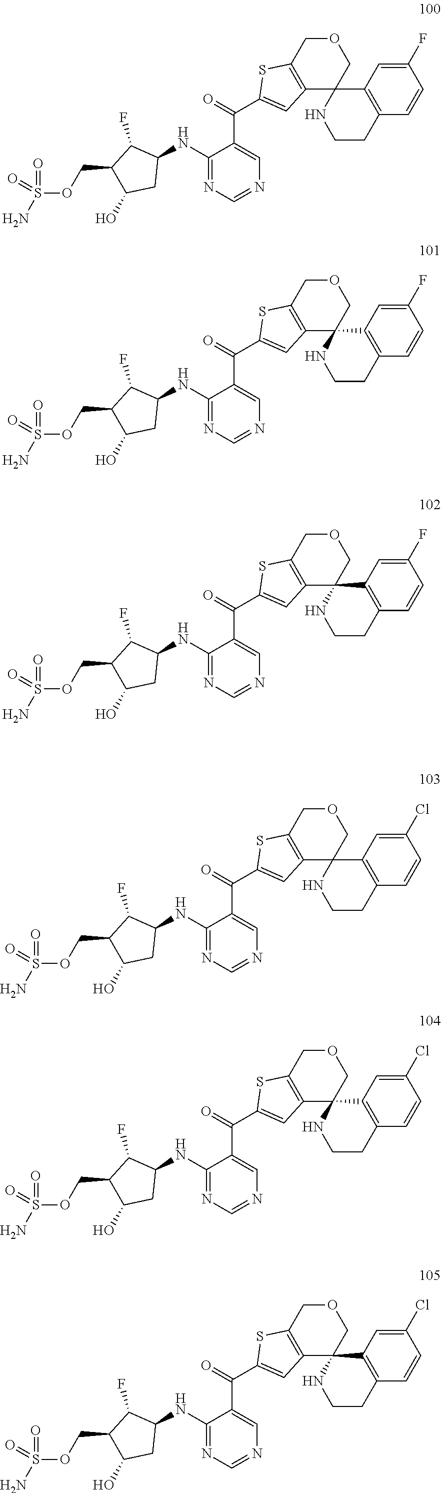

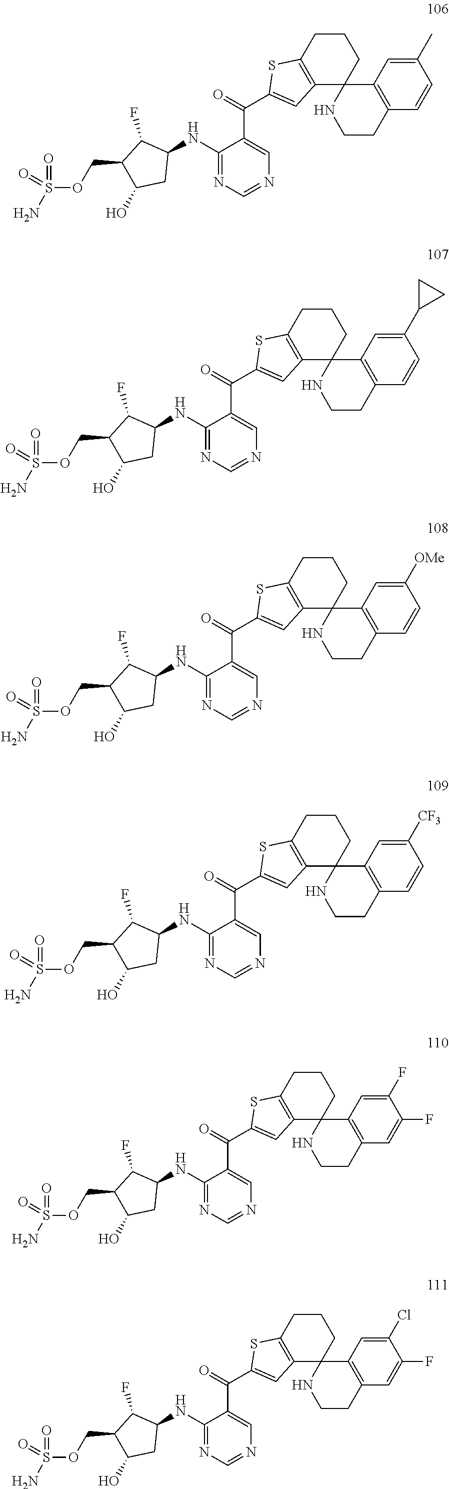

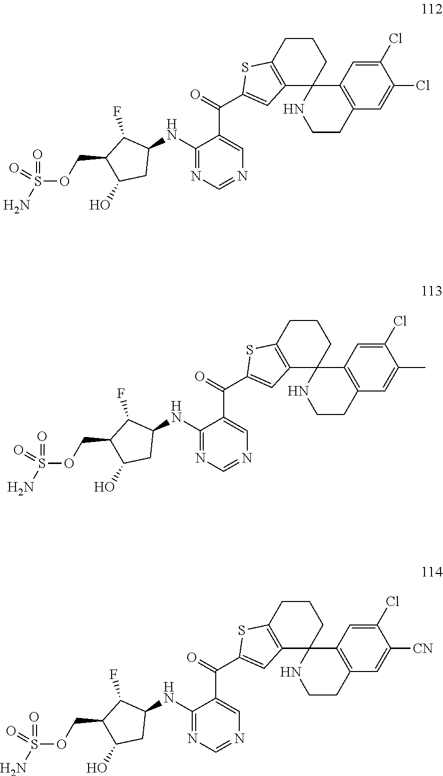

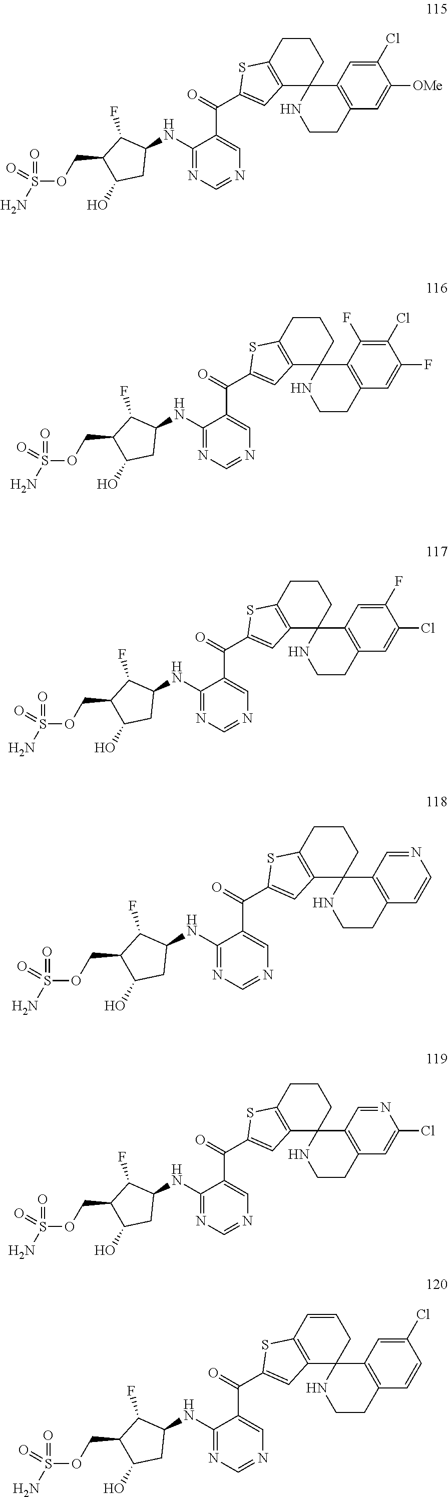

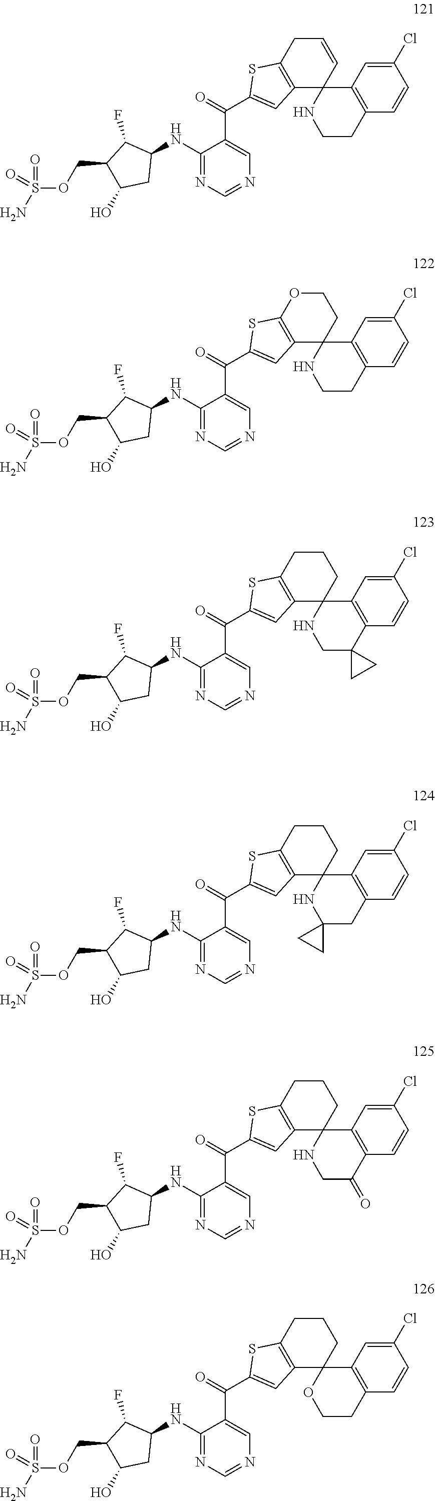

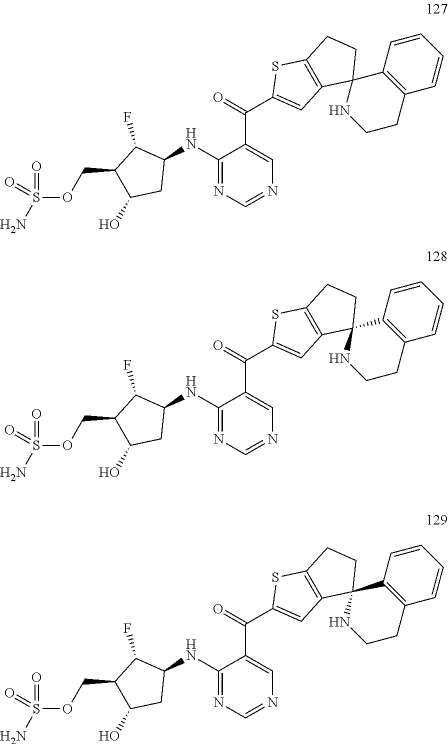

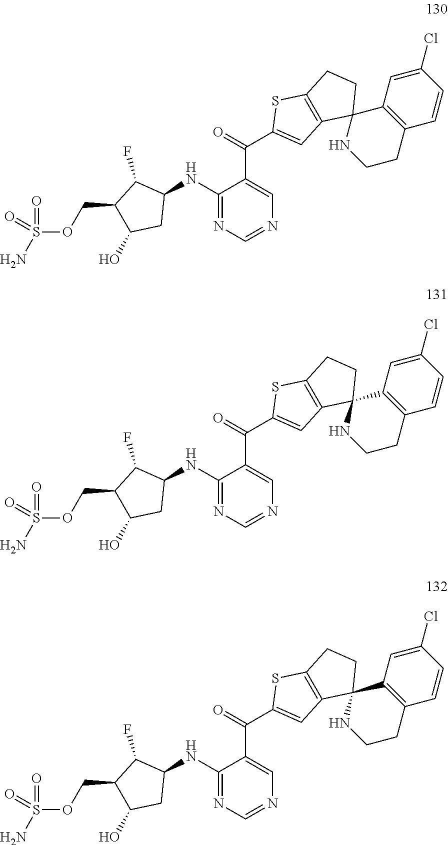

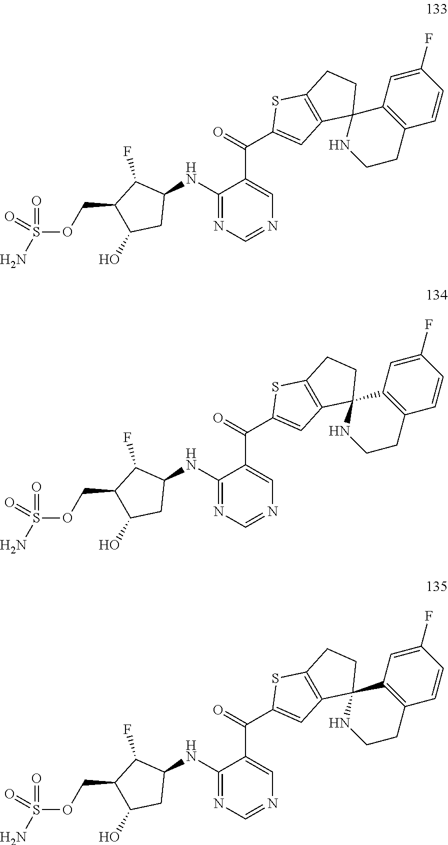

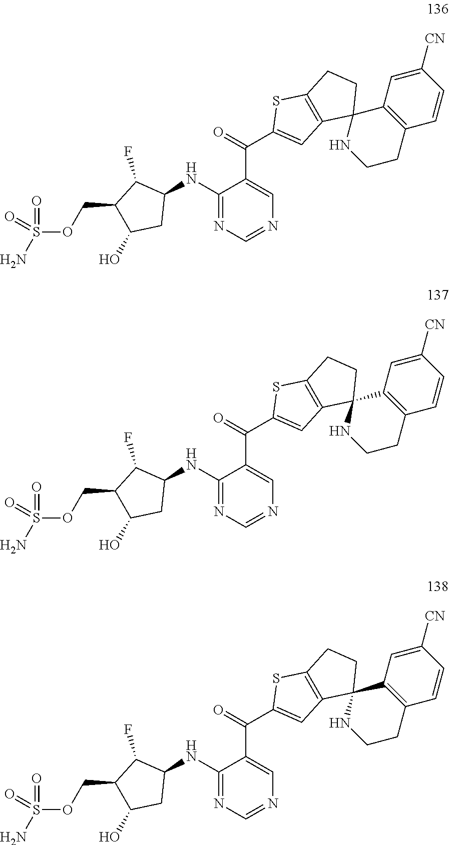

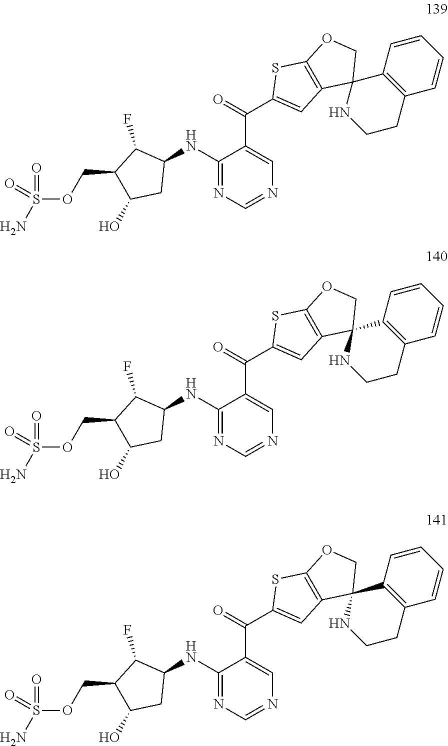

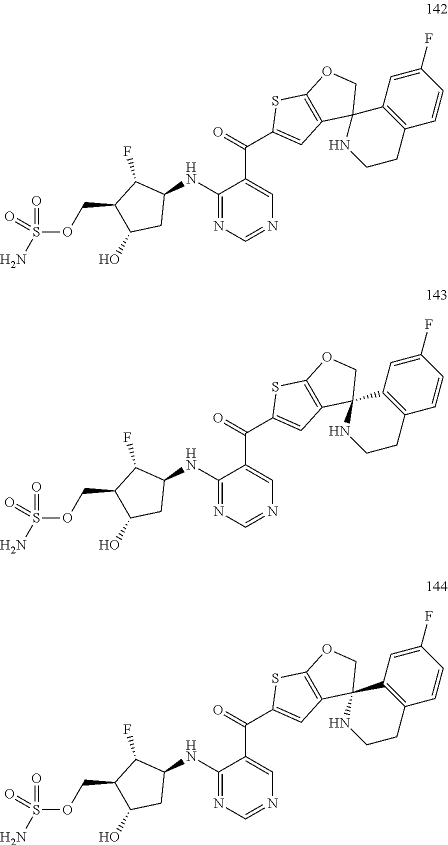

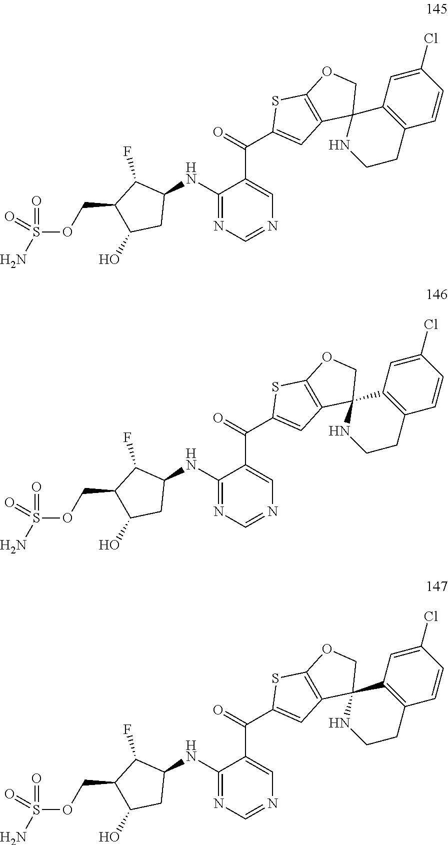

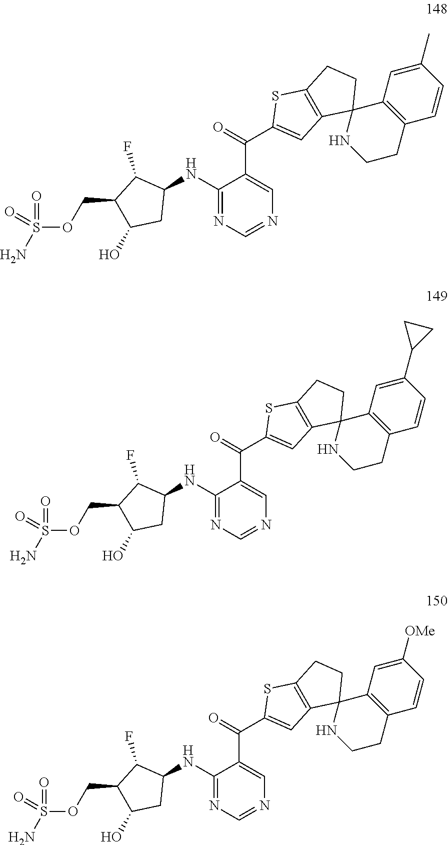

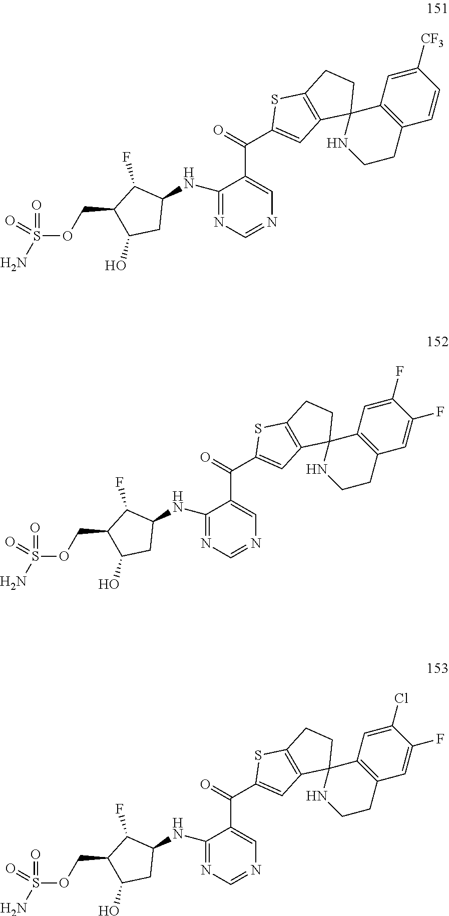

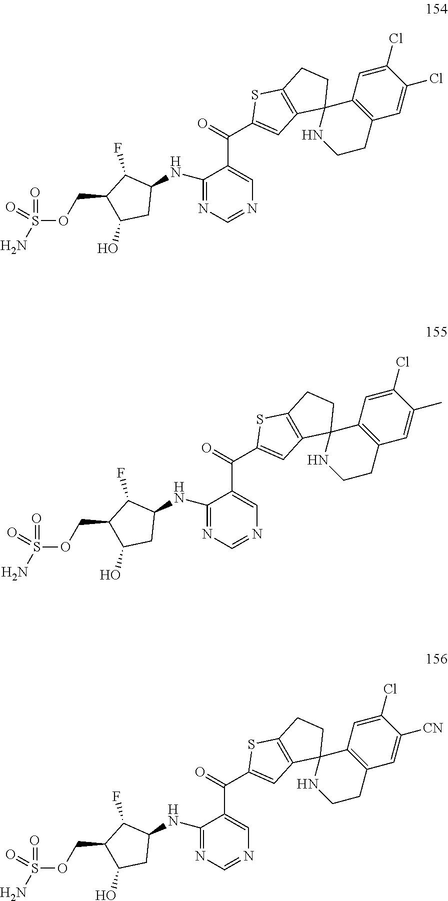

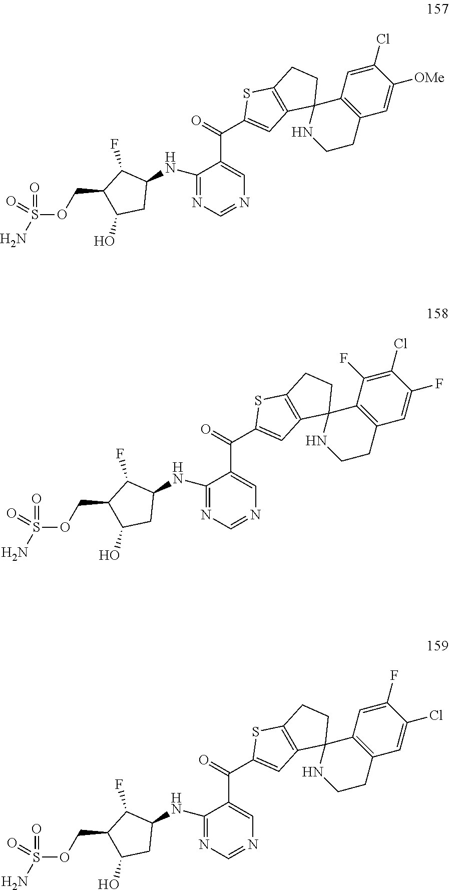

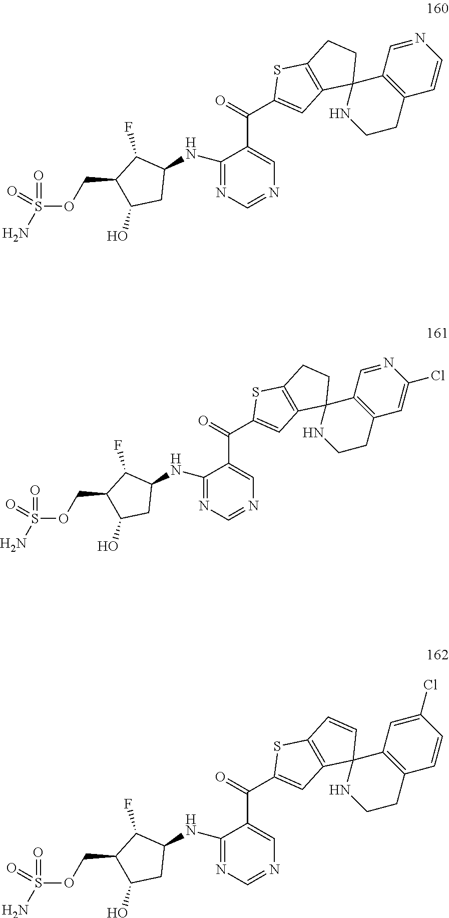

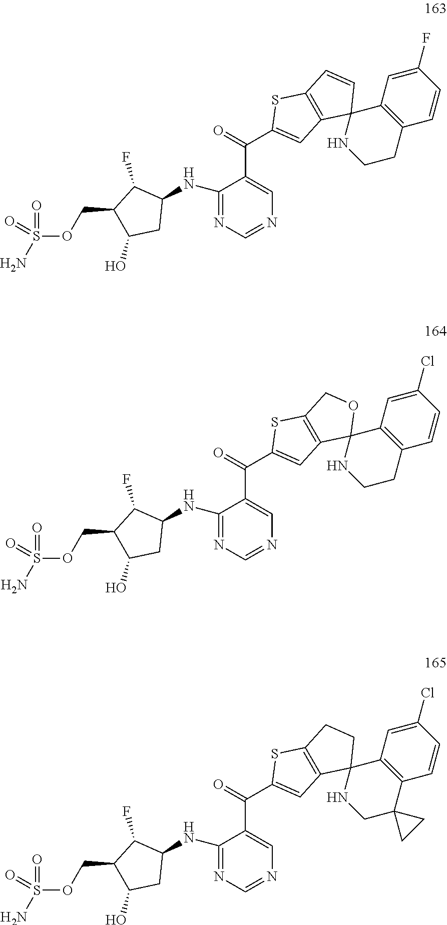

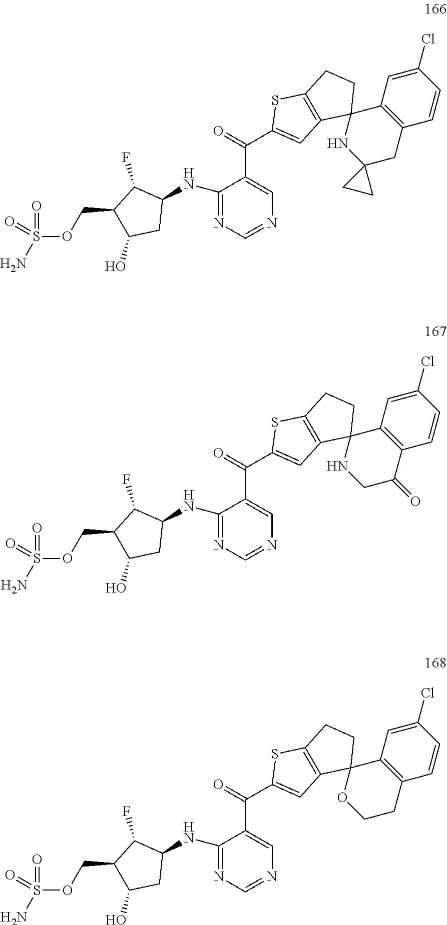

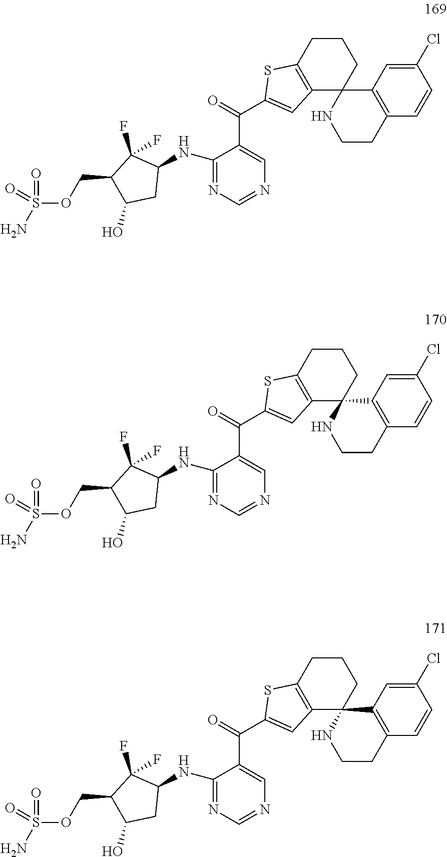

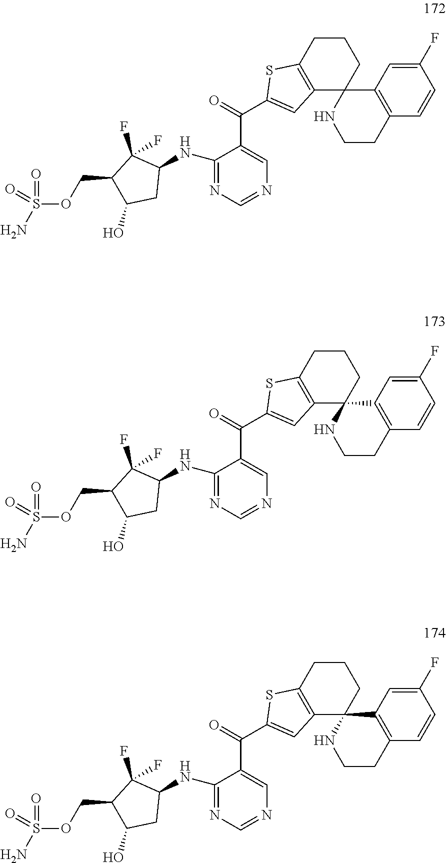

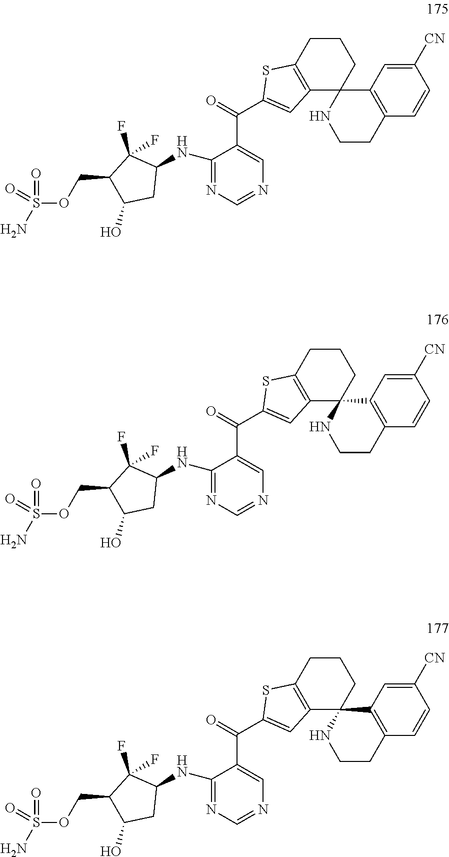

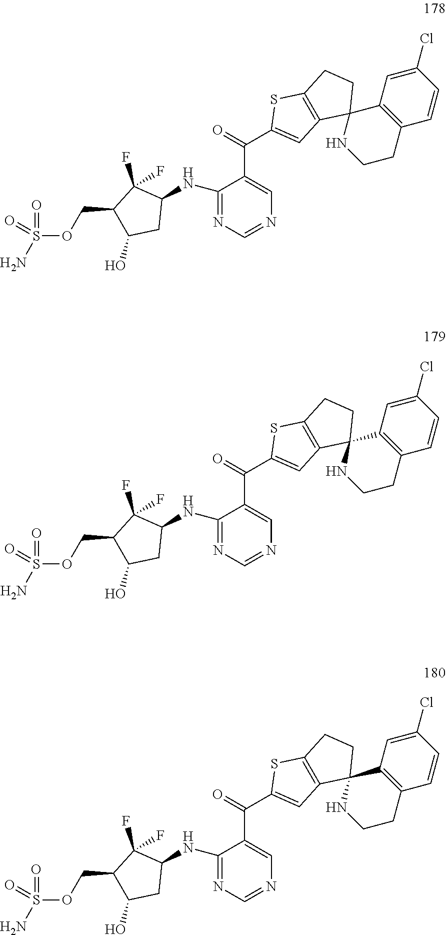

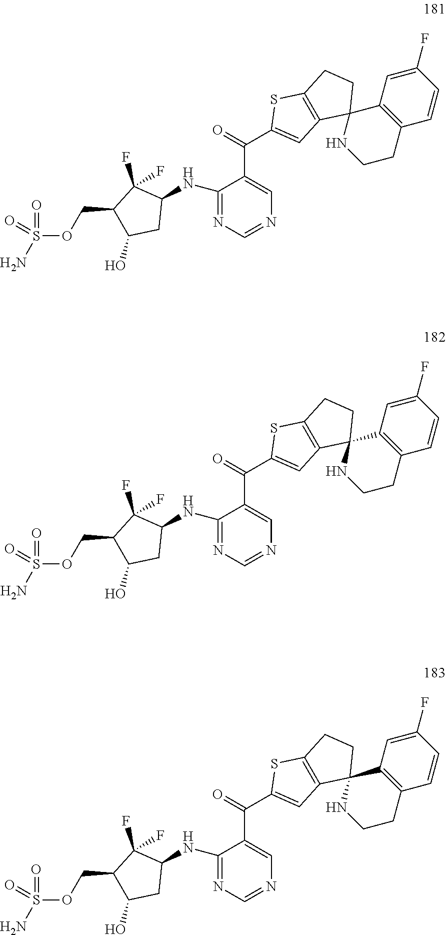

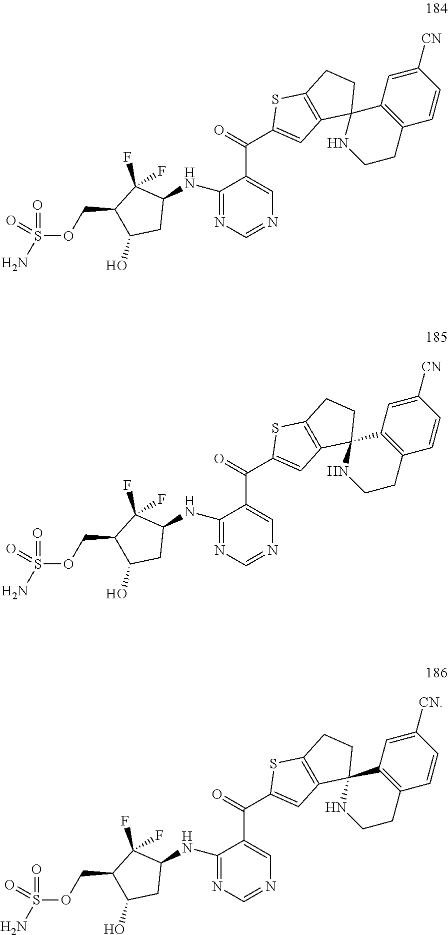

In another specific embodiment of the present disclosure, the compound of general formula (1) has one of the following structures

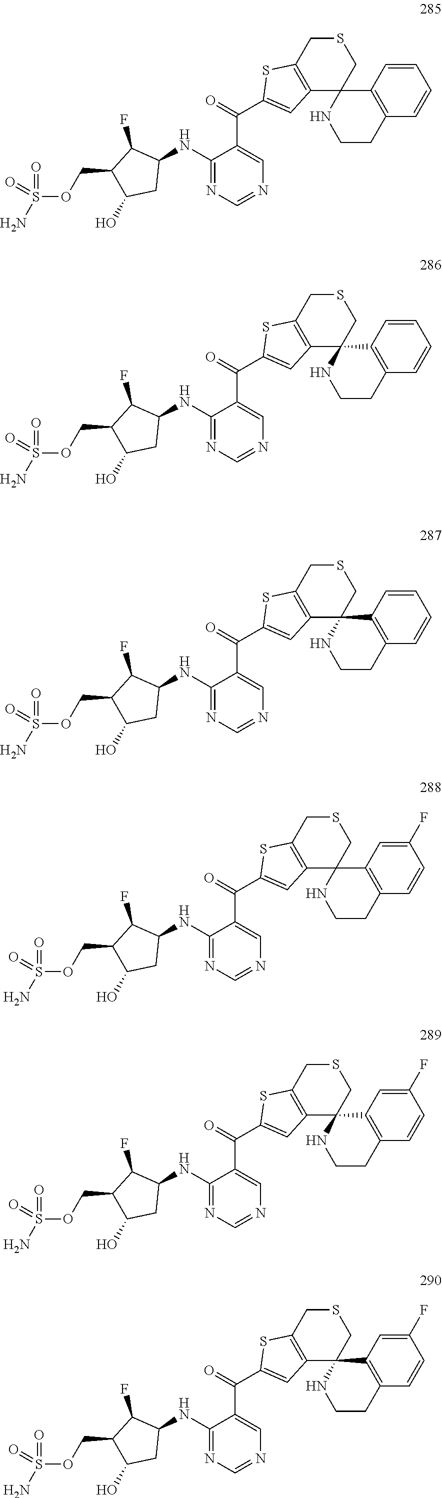

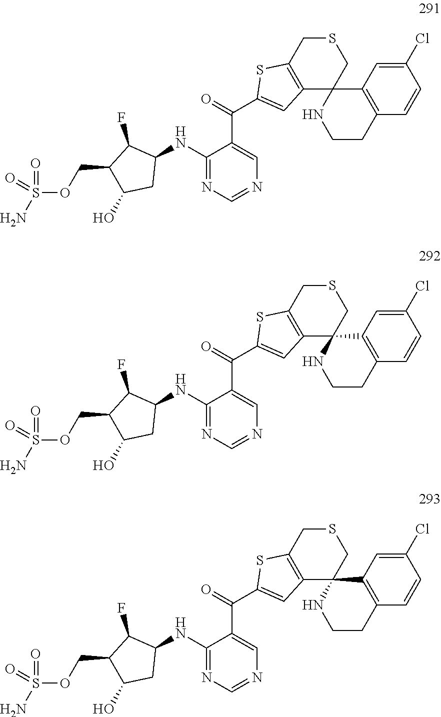

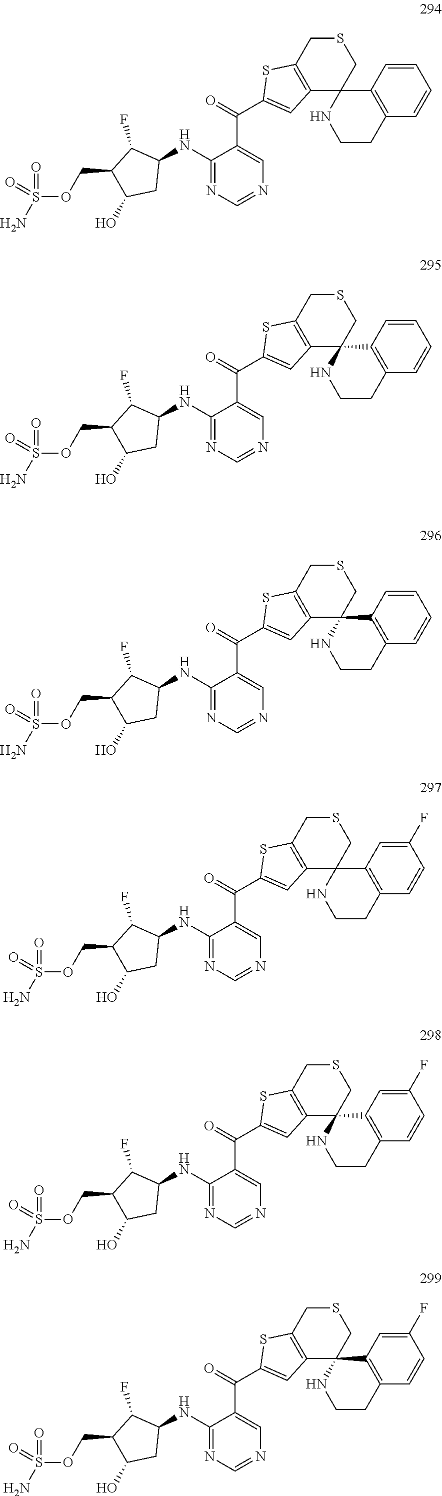

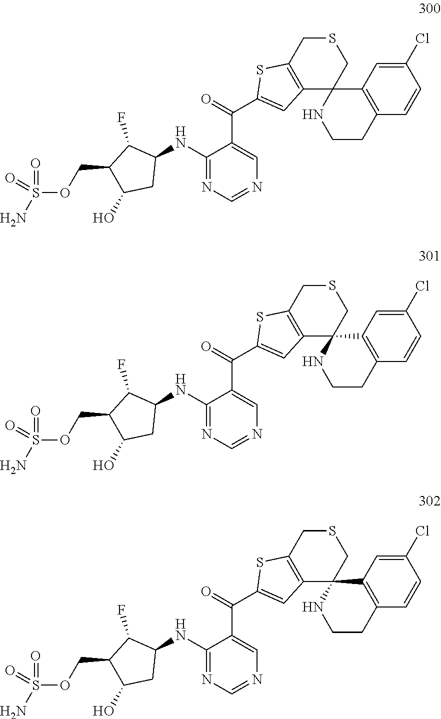

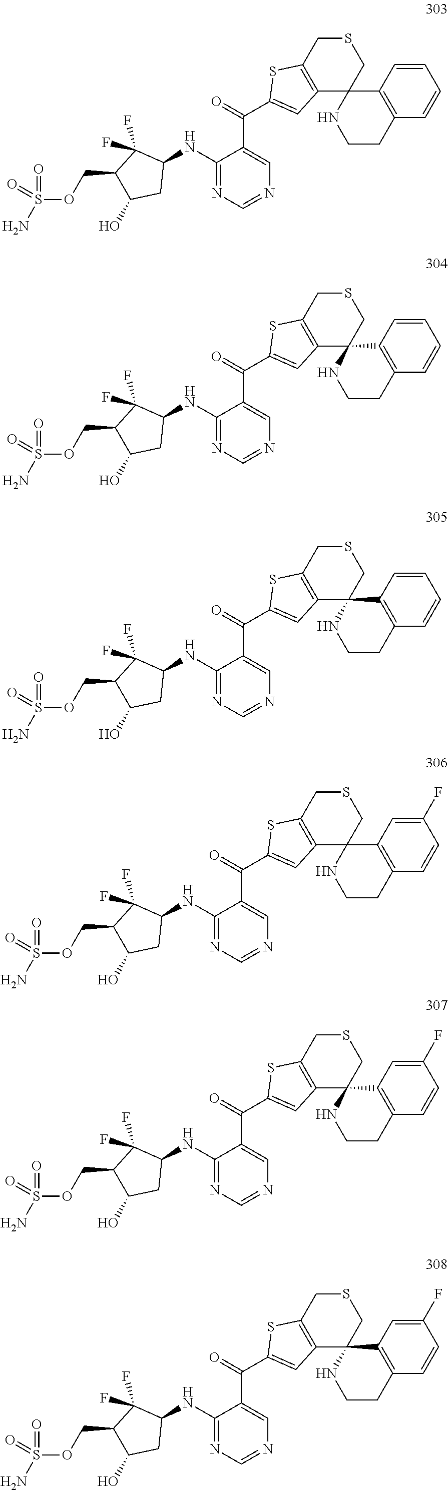

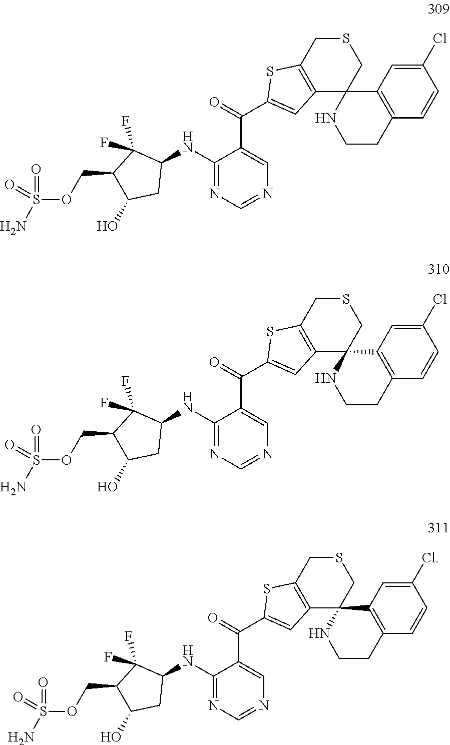

In another specific embodiment of the present disclosure, the compound of general formula (1) has one of the following structures

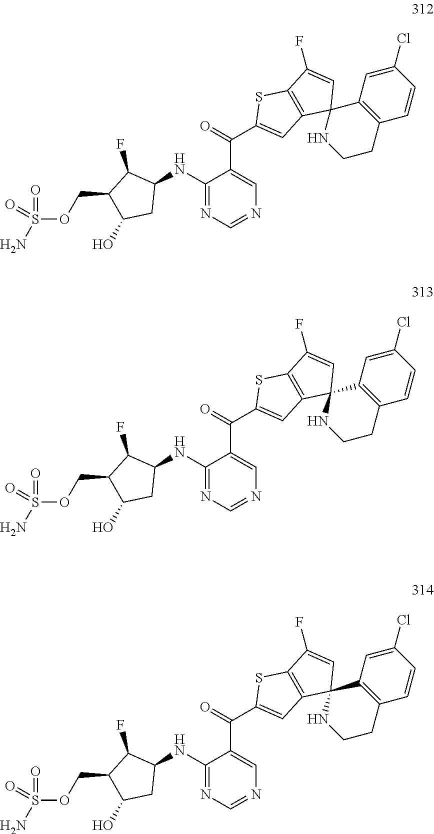

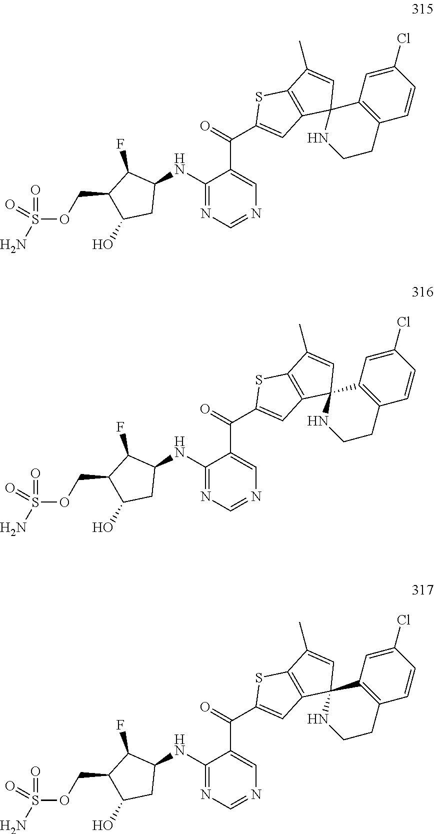

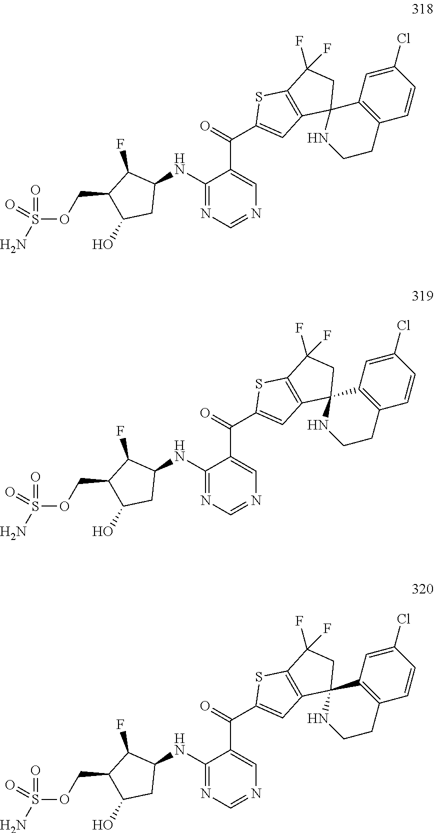

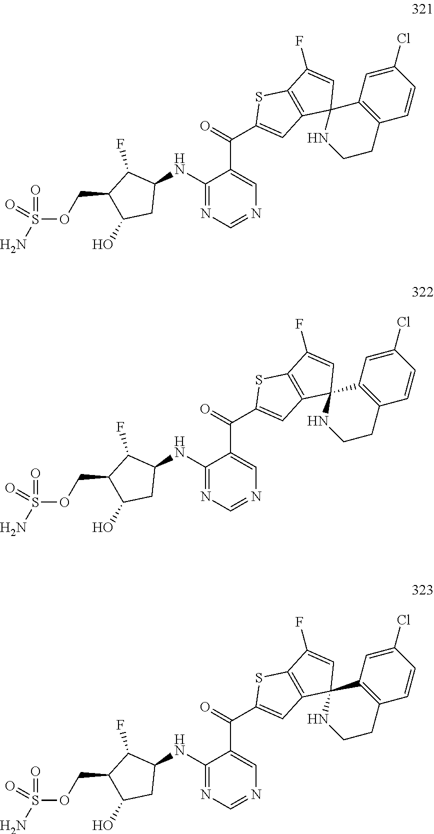

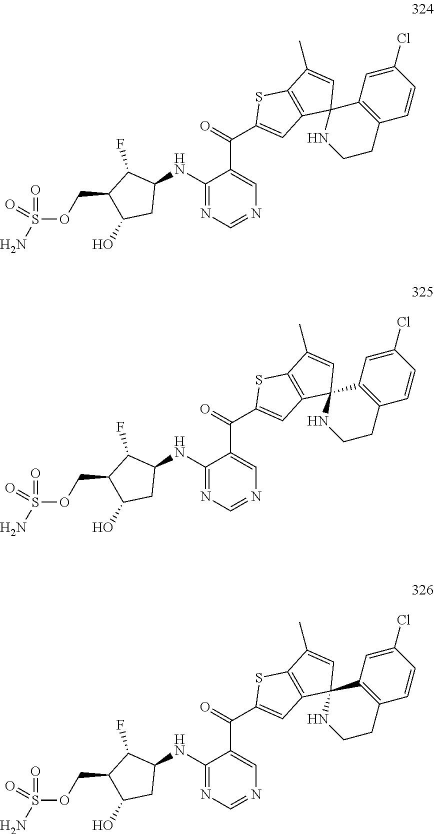

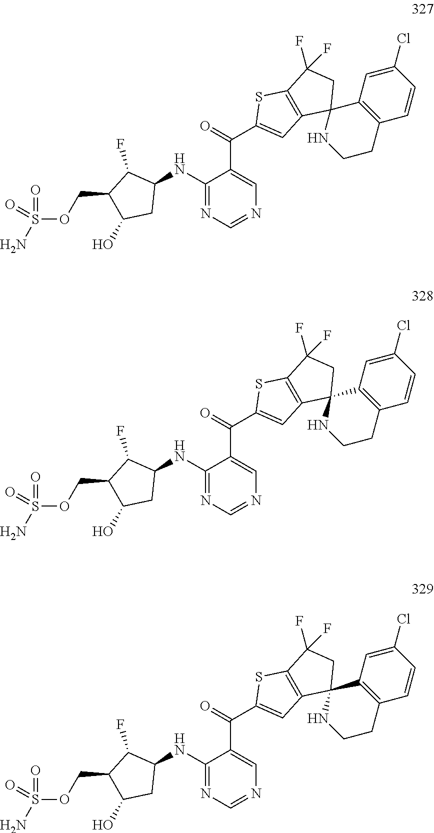

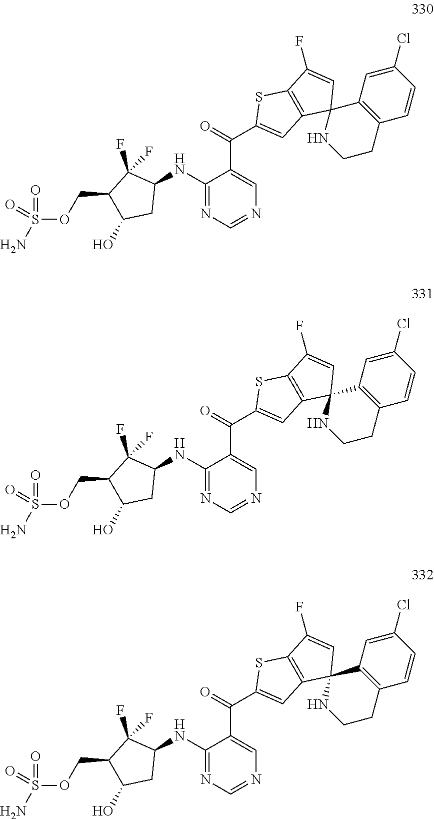

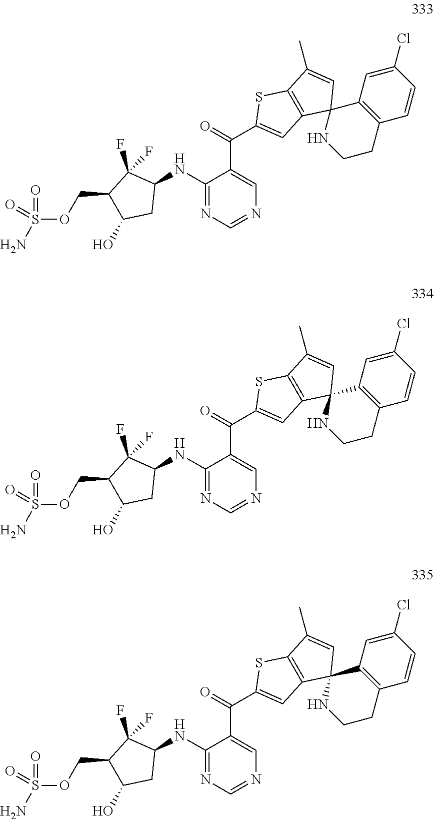

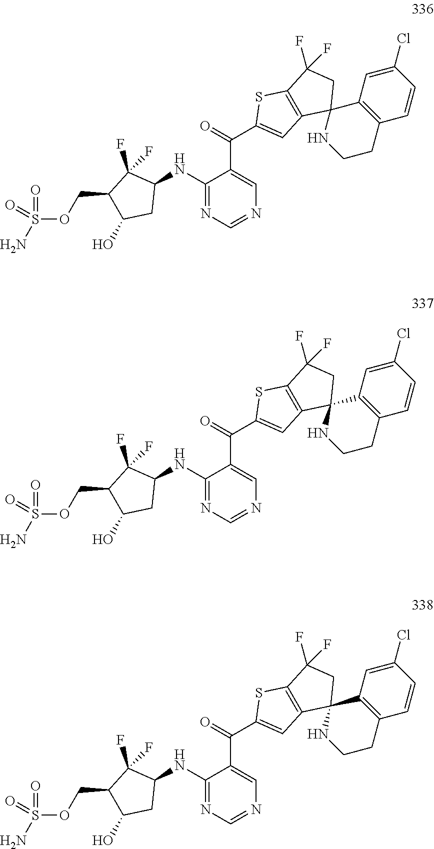

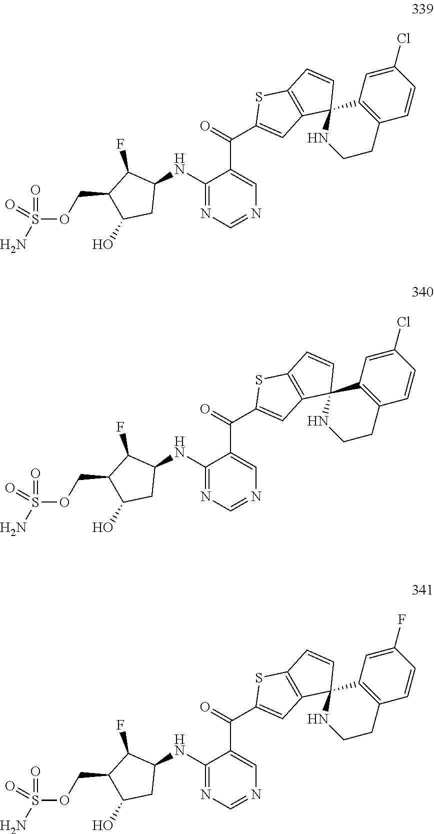

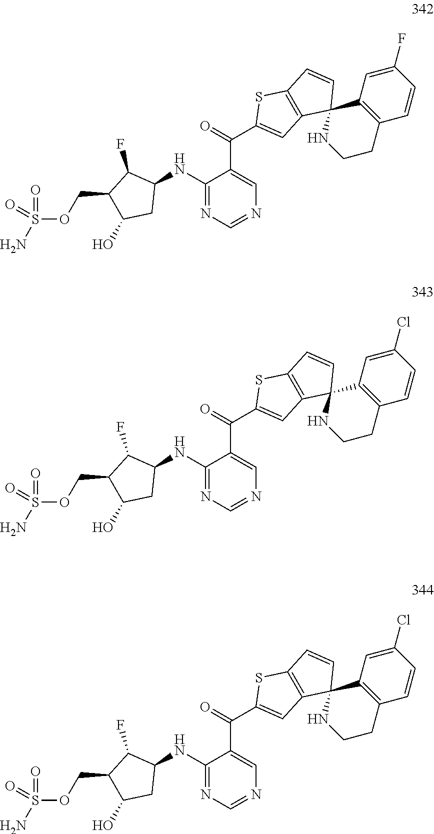

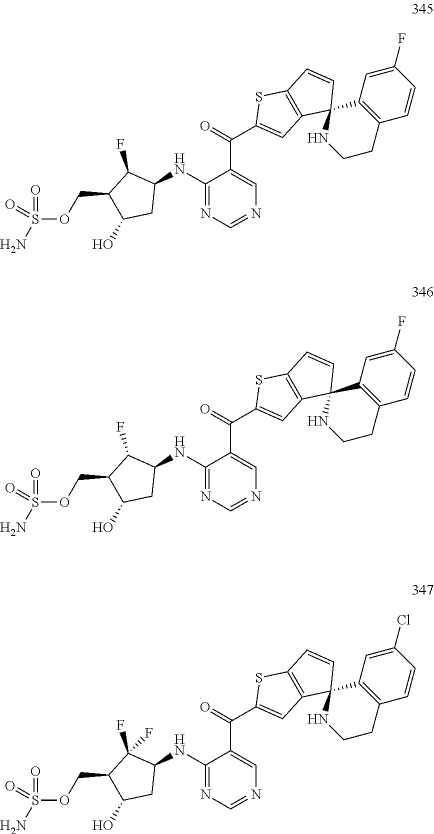

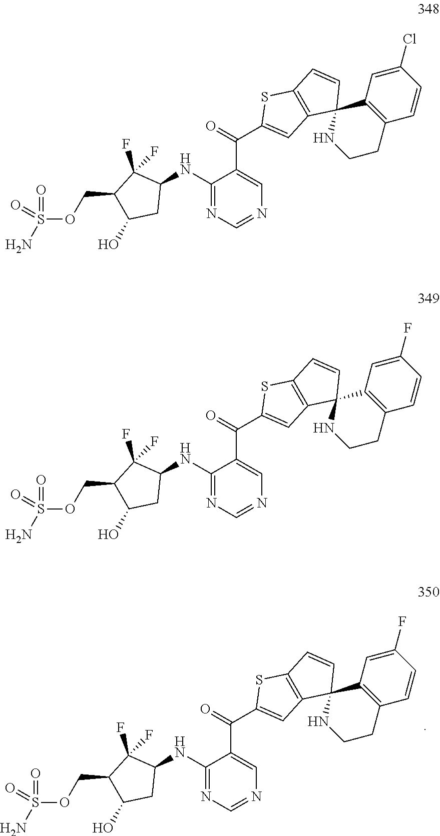

In another specific embodiment of the present disclosure, the compound of general formula (1) has one of the following structures

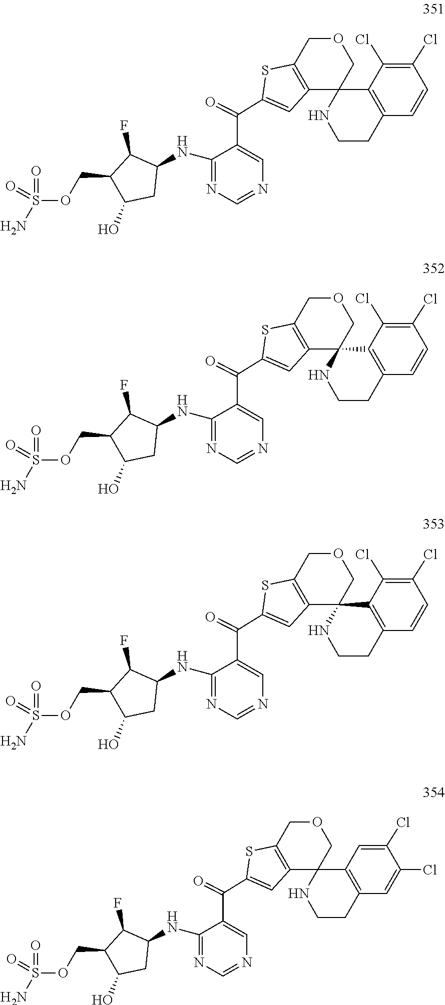

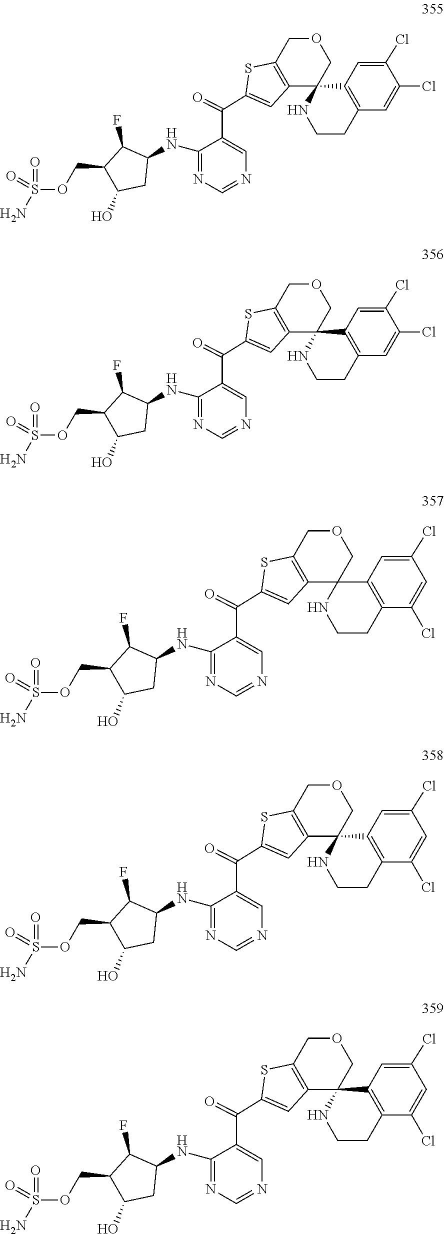

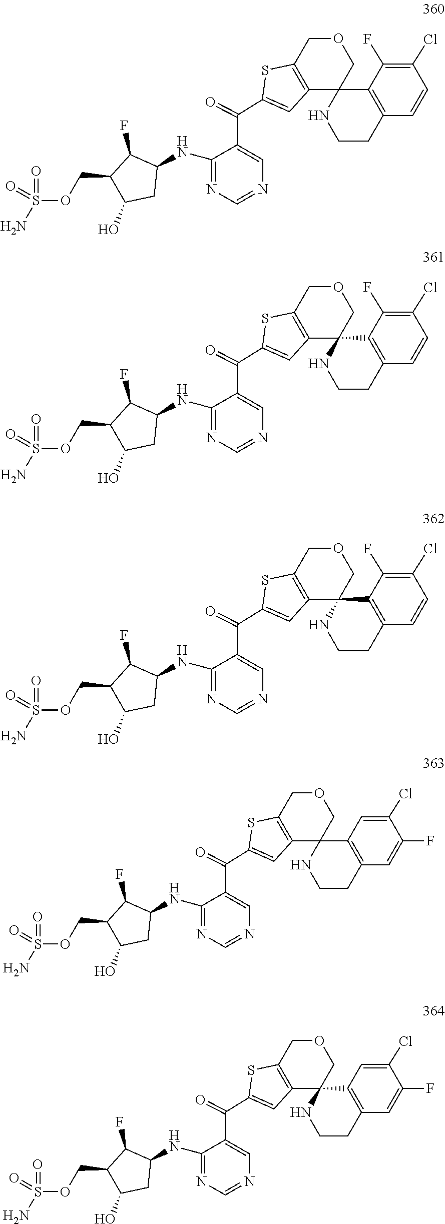

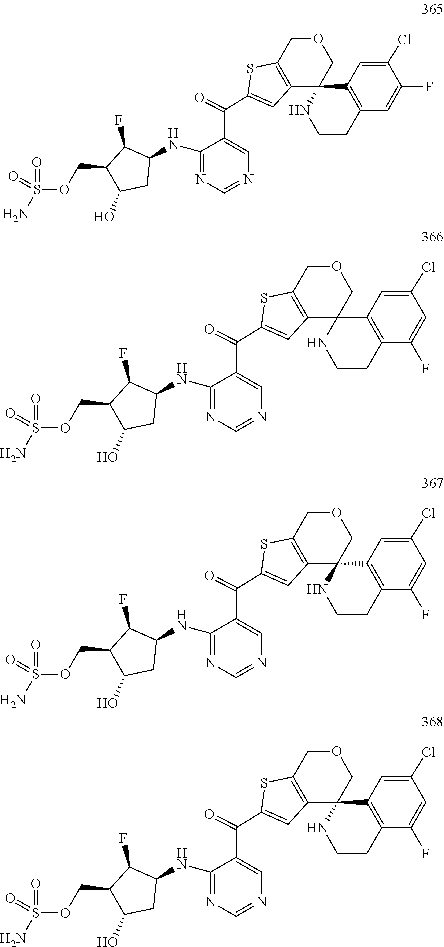

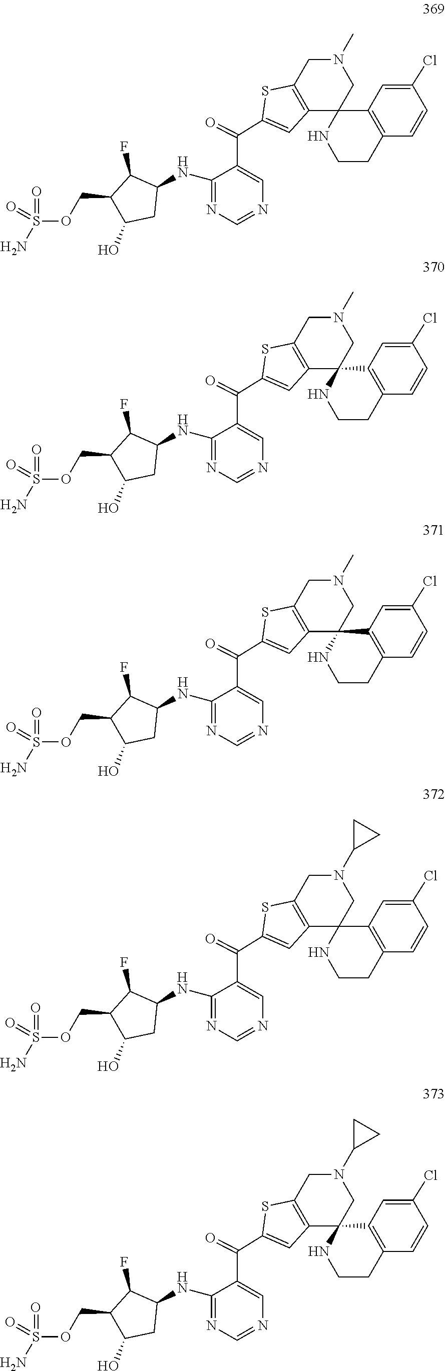

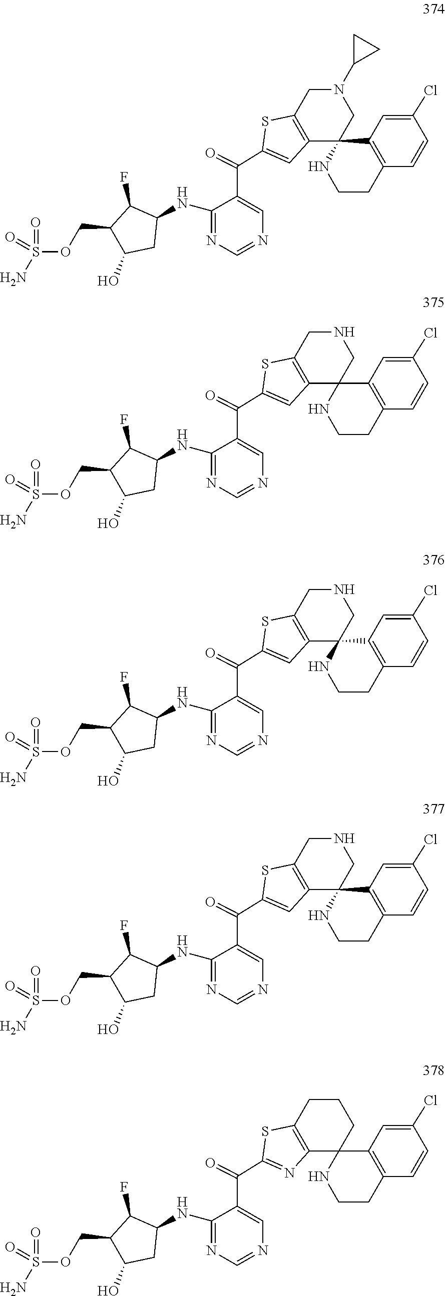

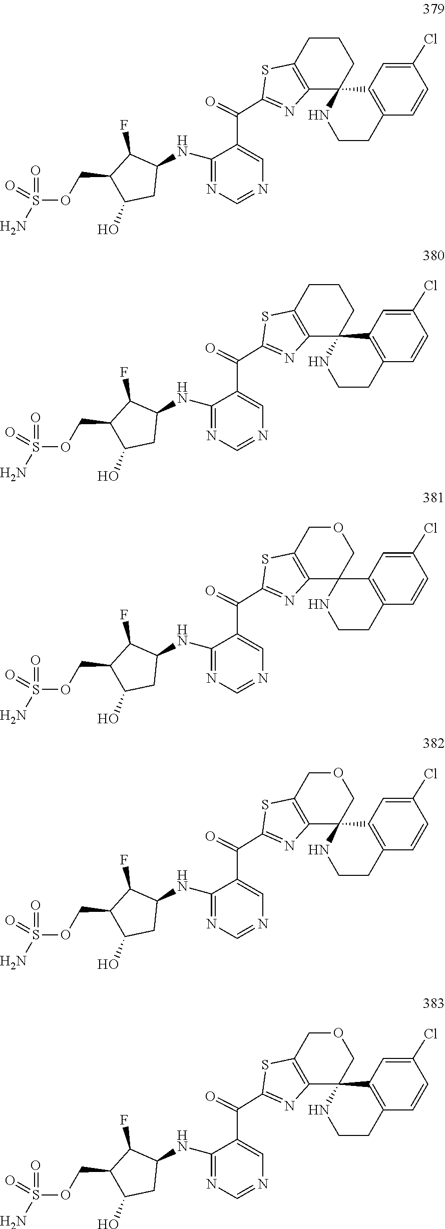

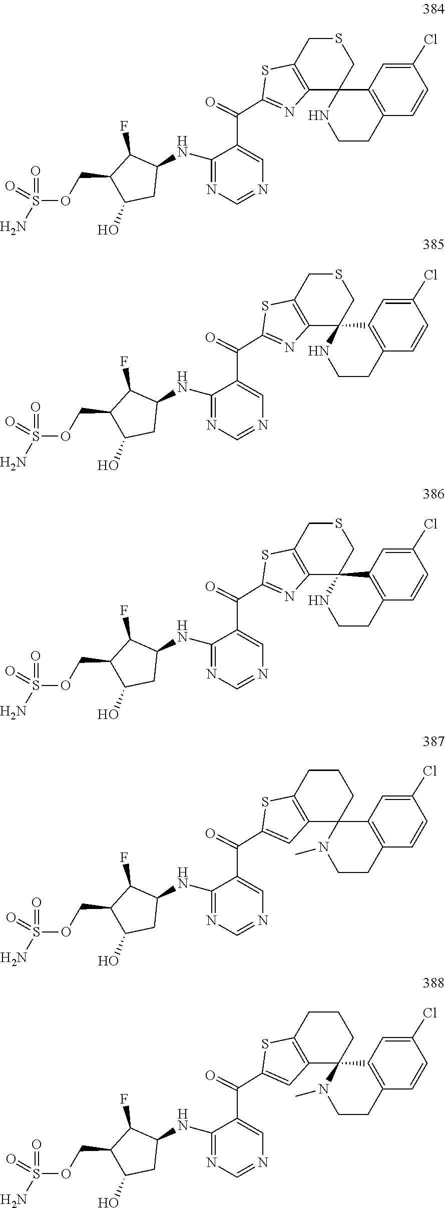

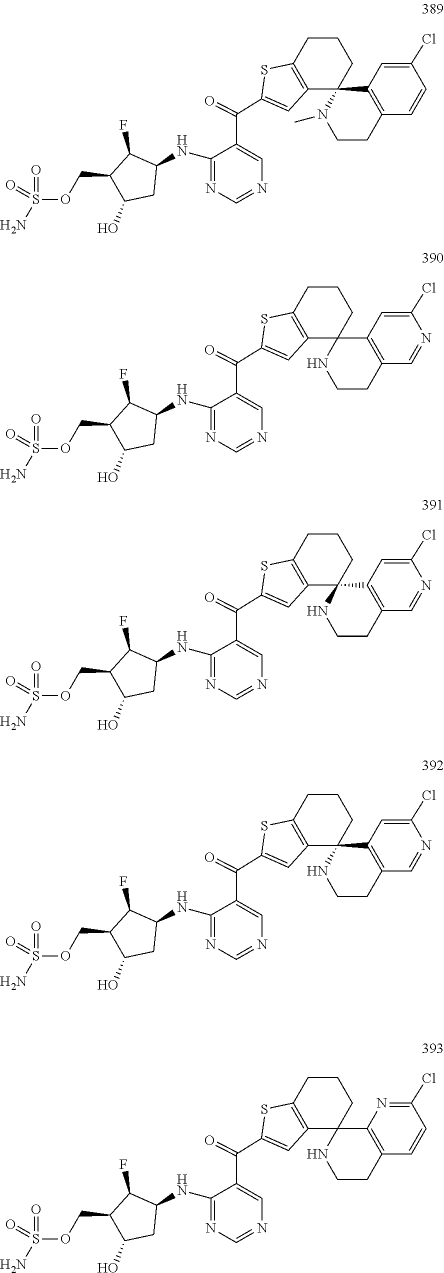

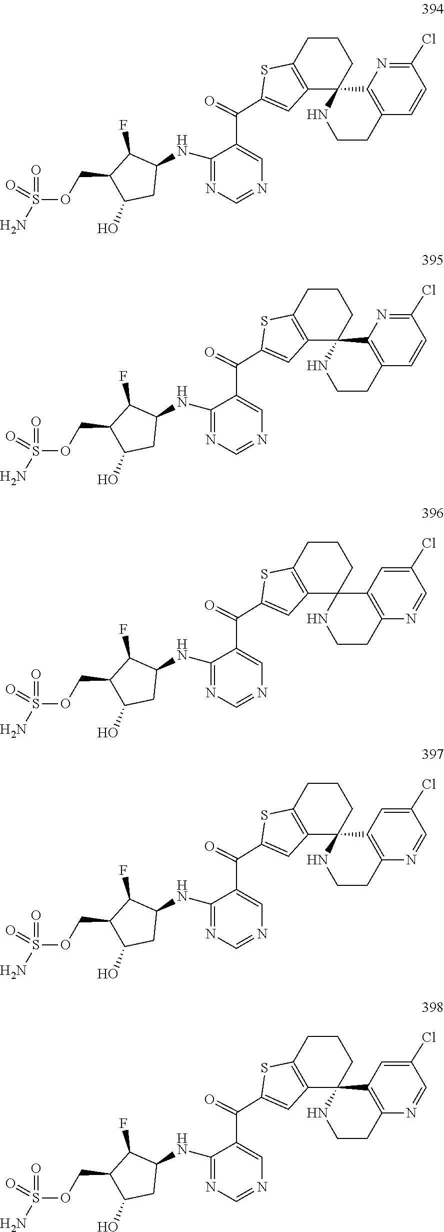

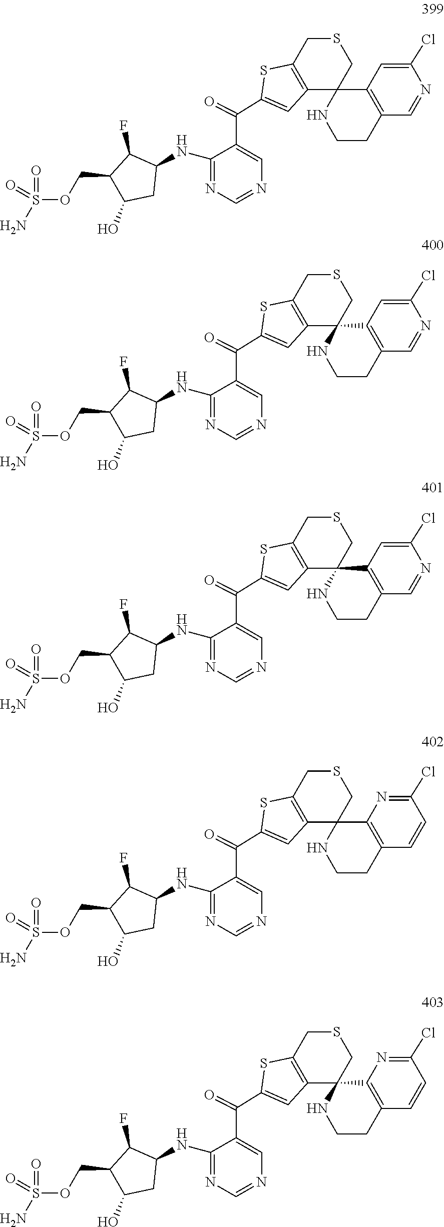

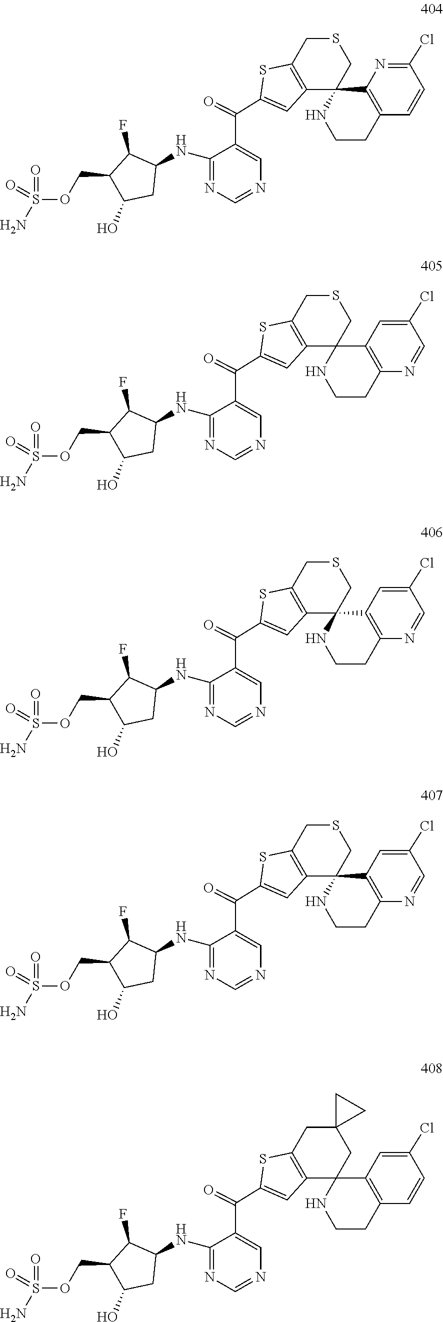

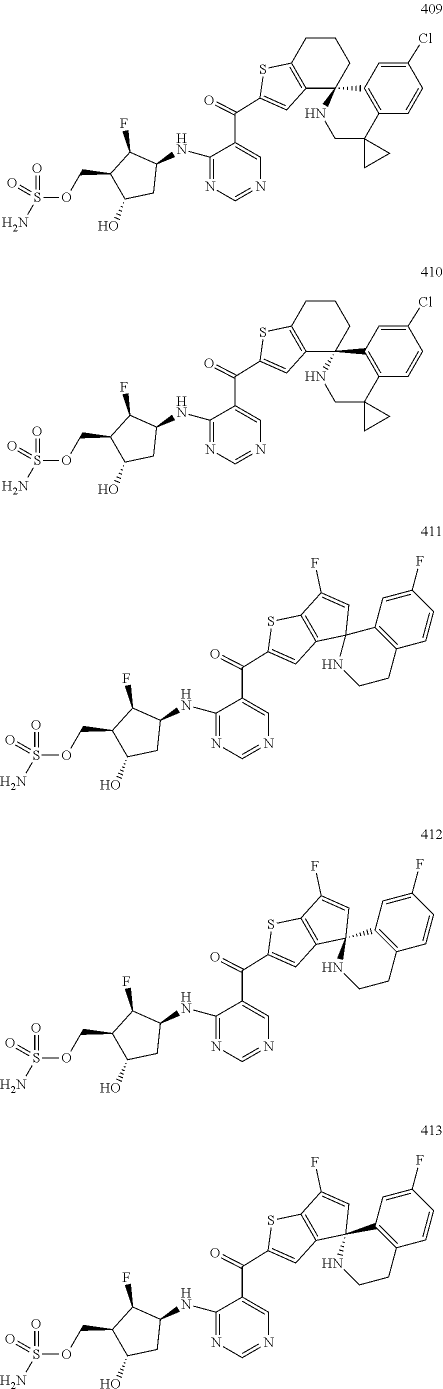

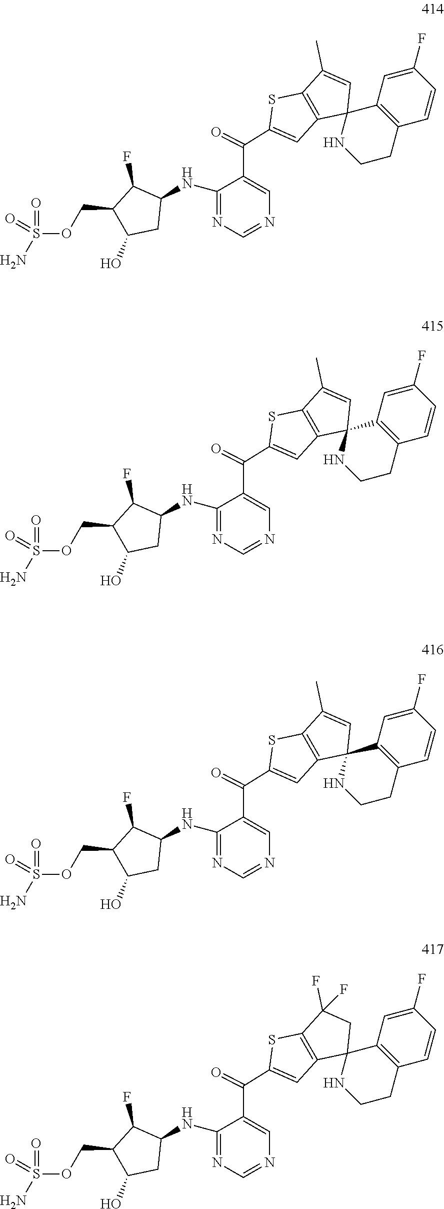

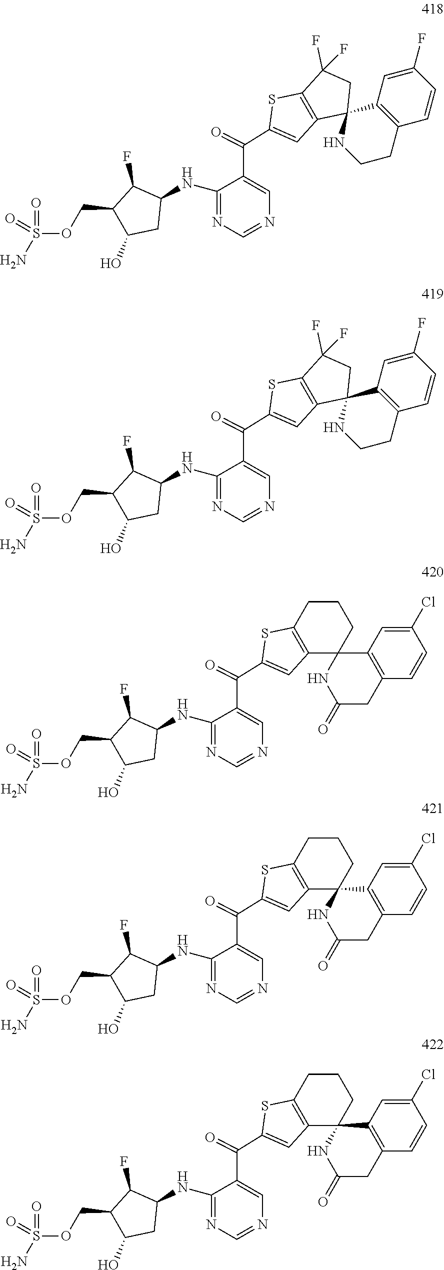

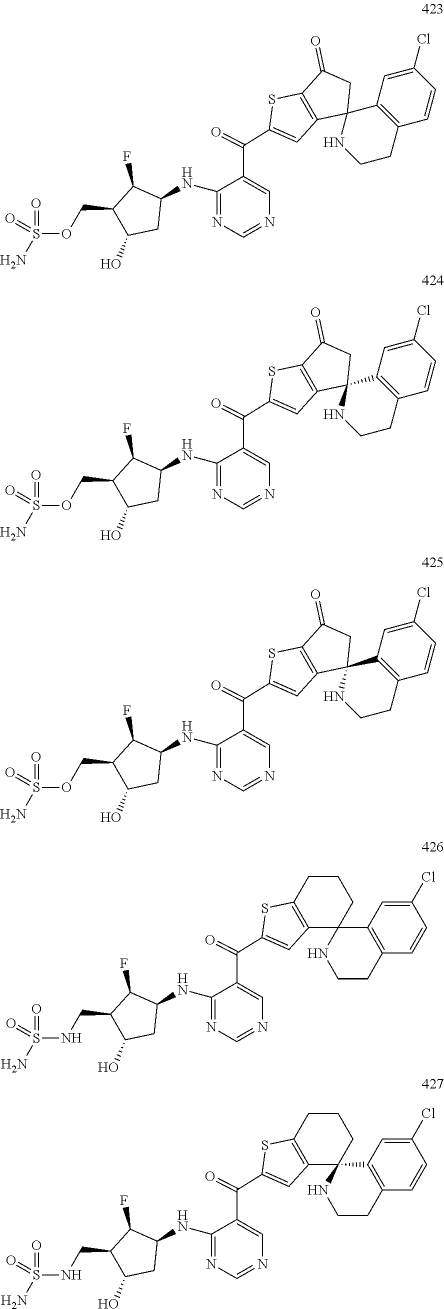

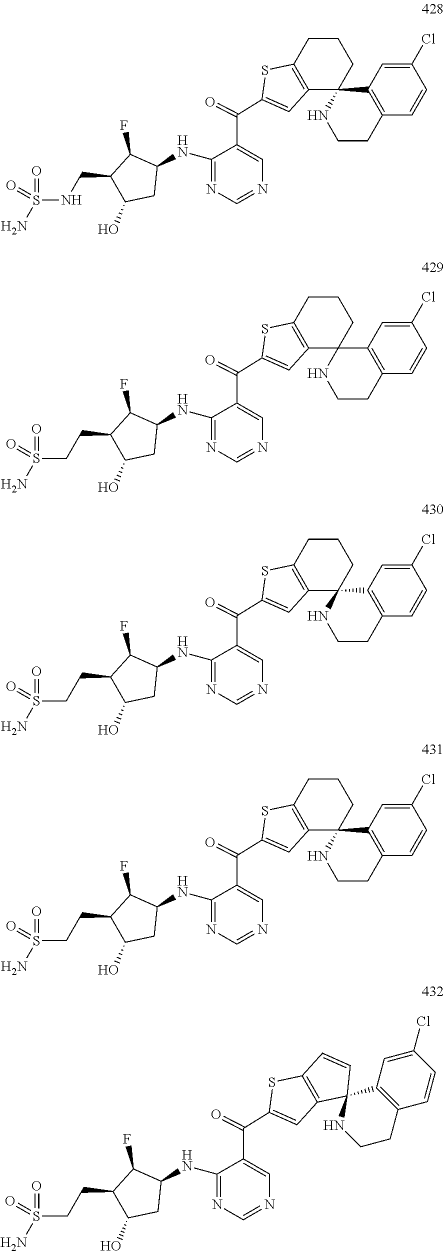

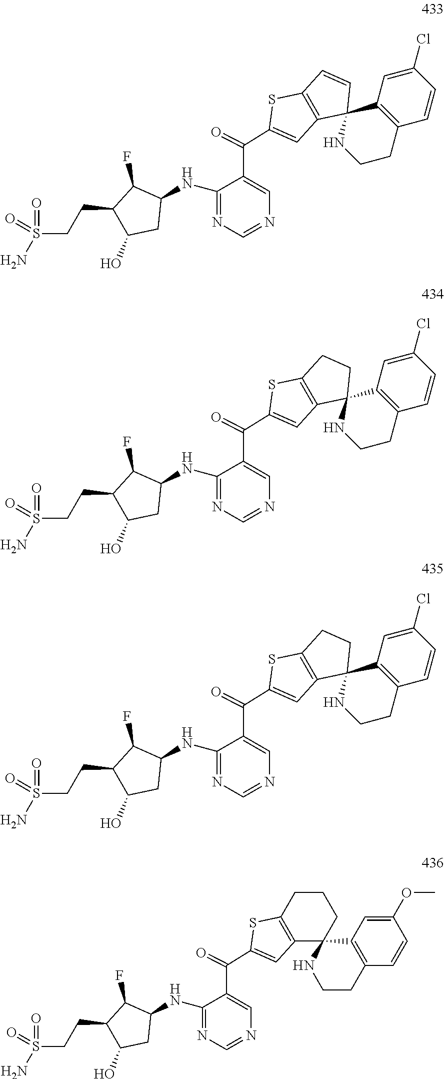

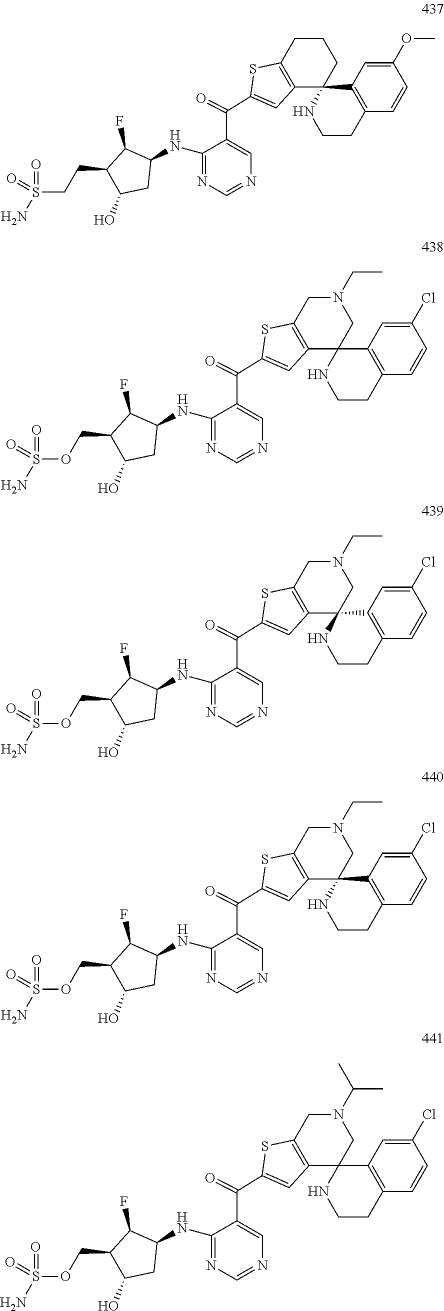

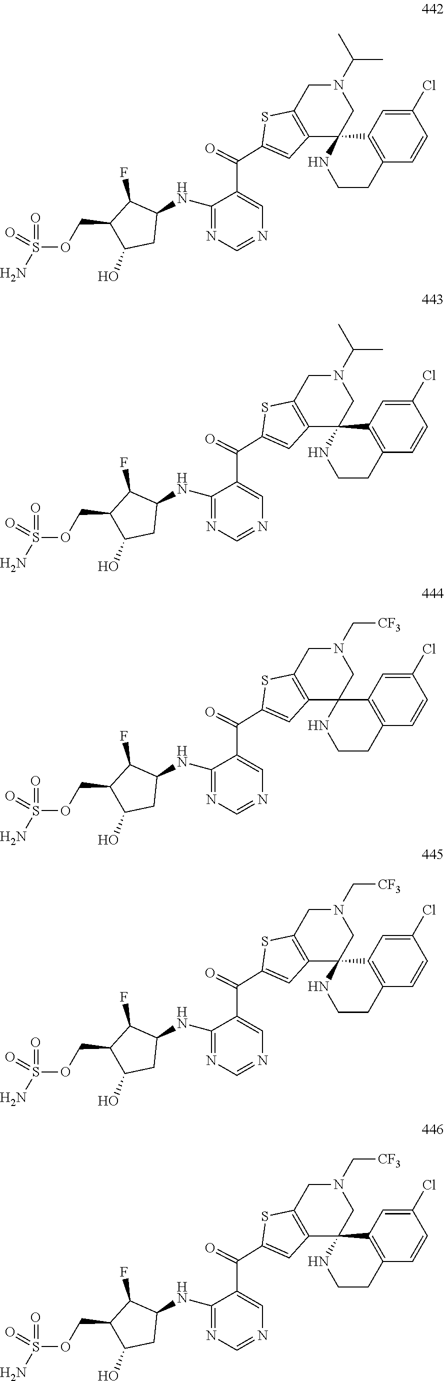

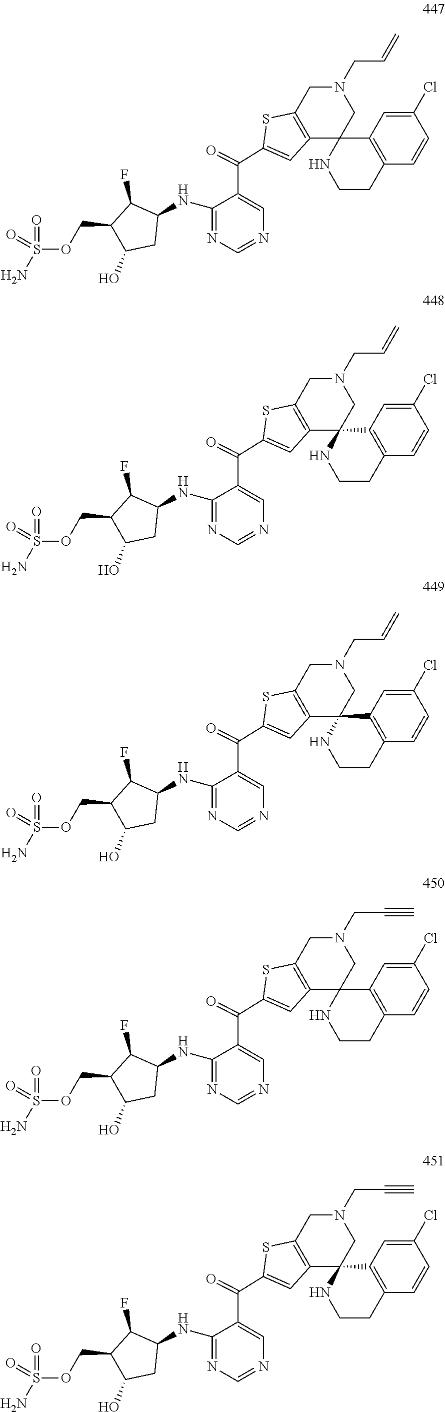

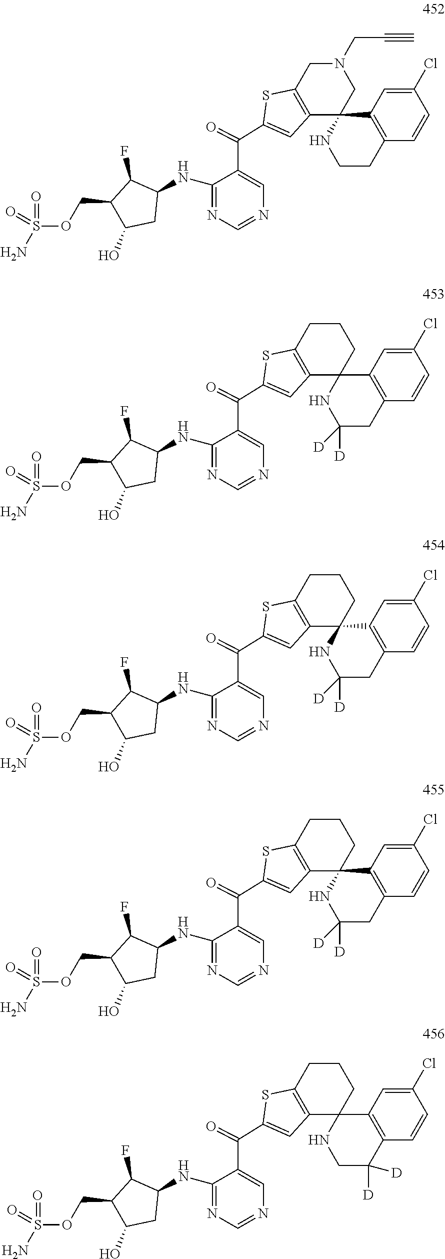

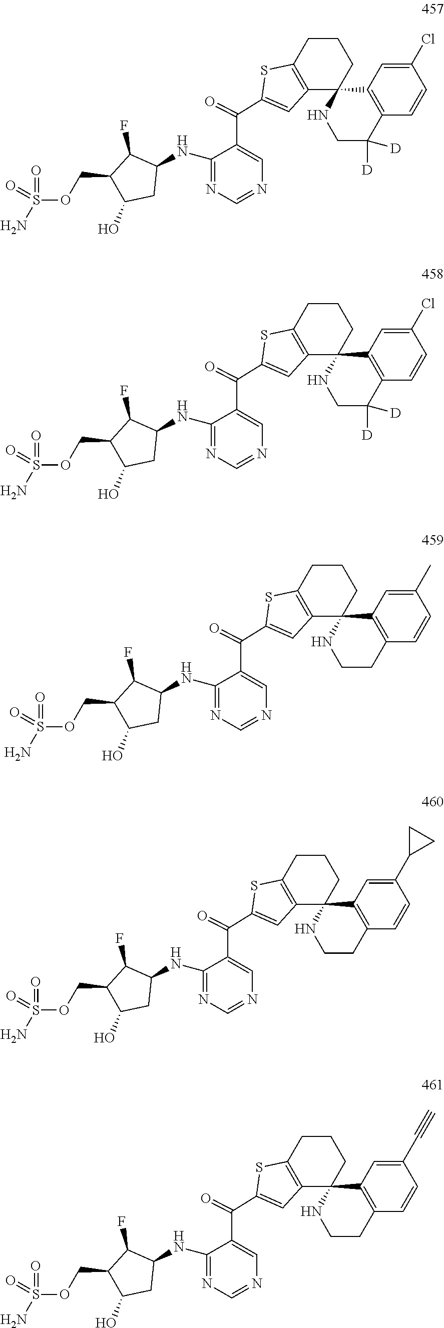

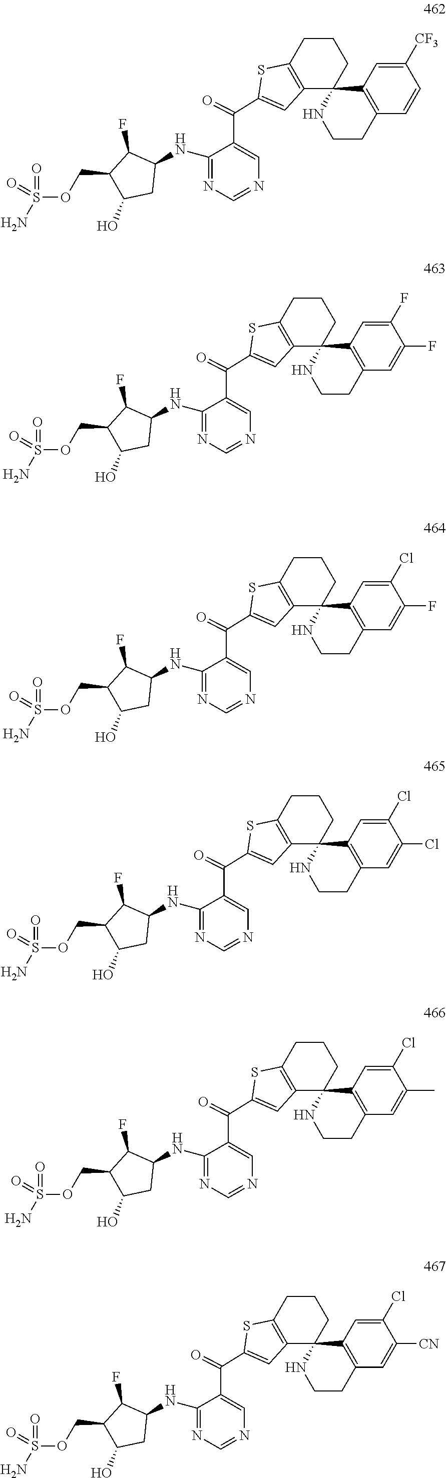

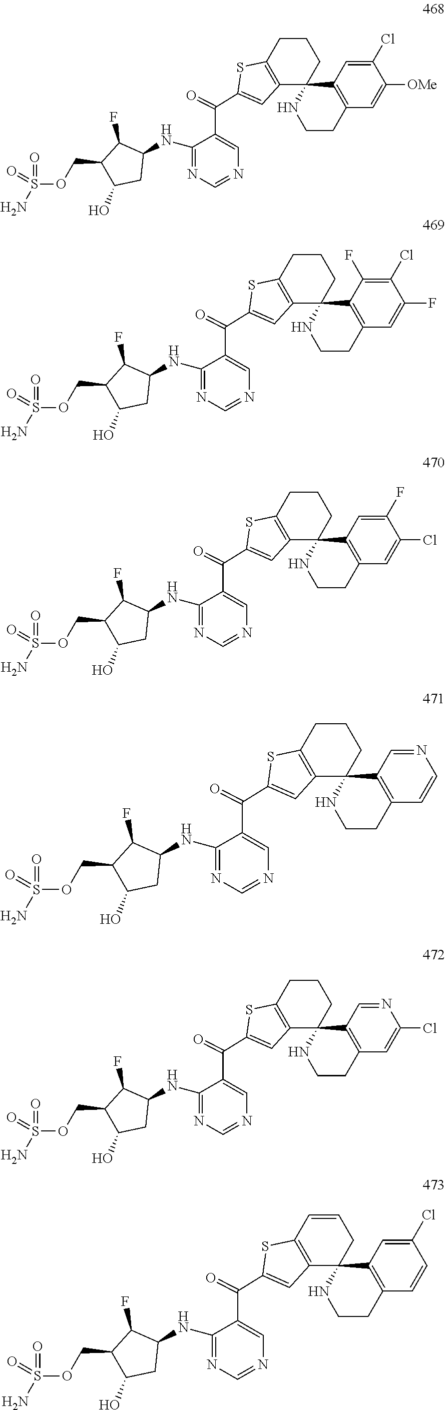

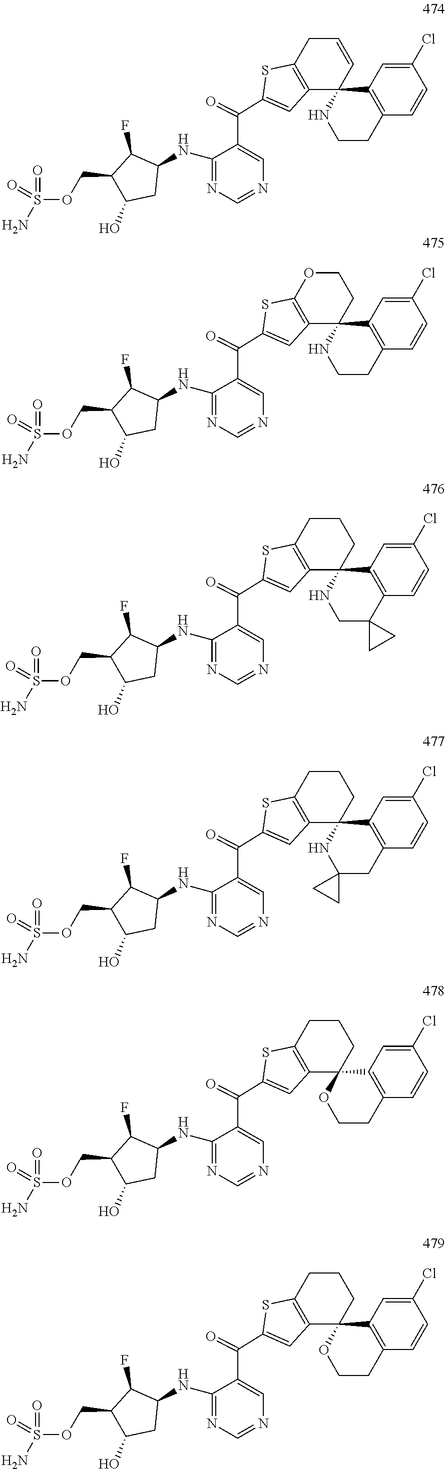

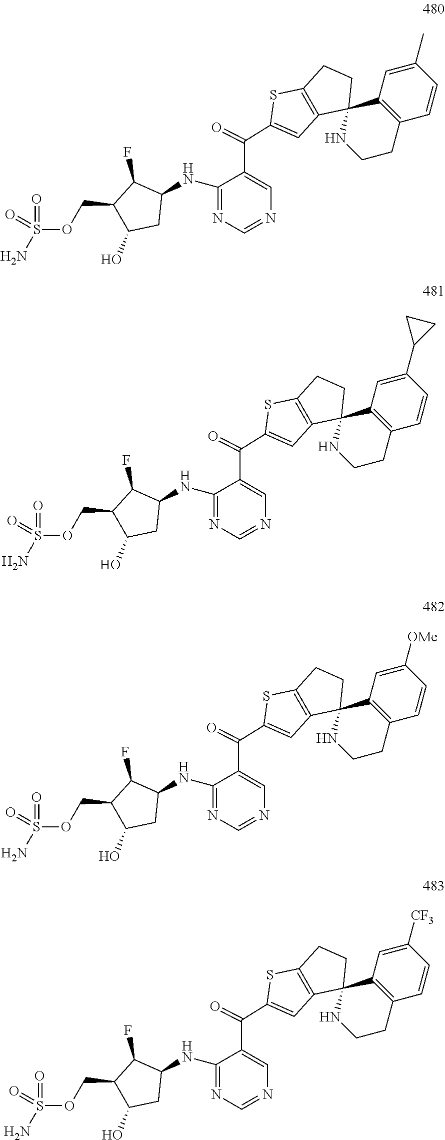

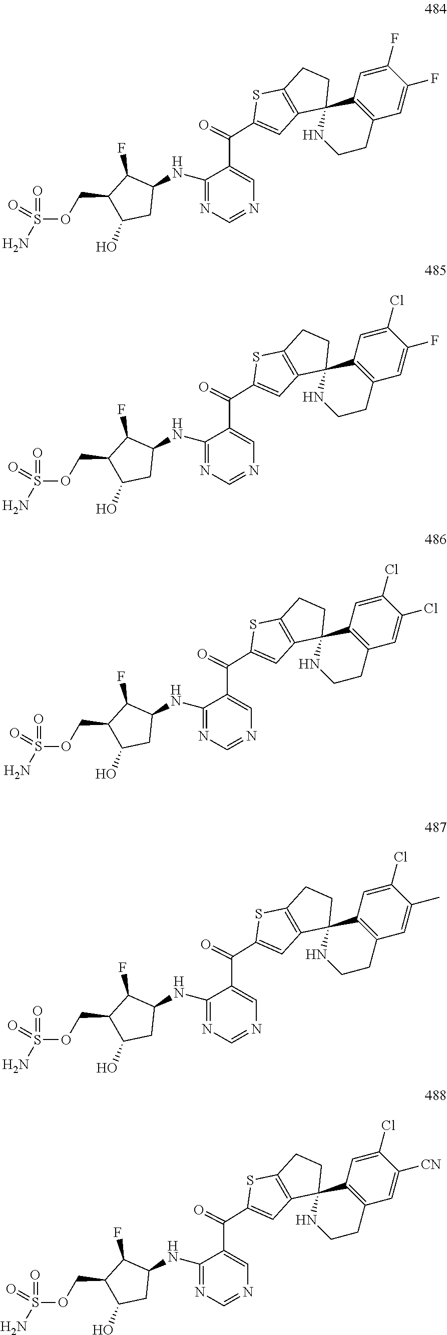

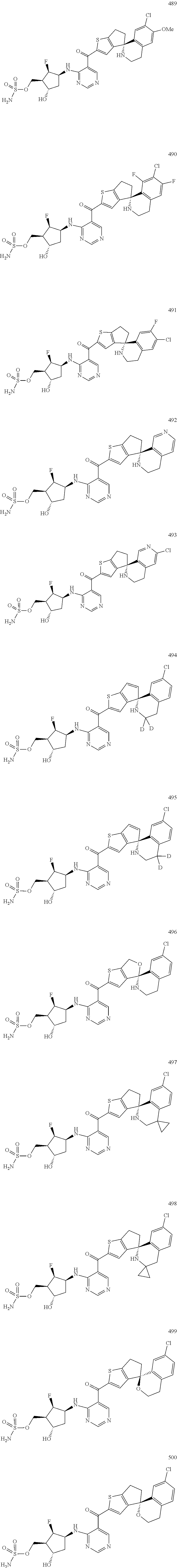

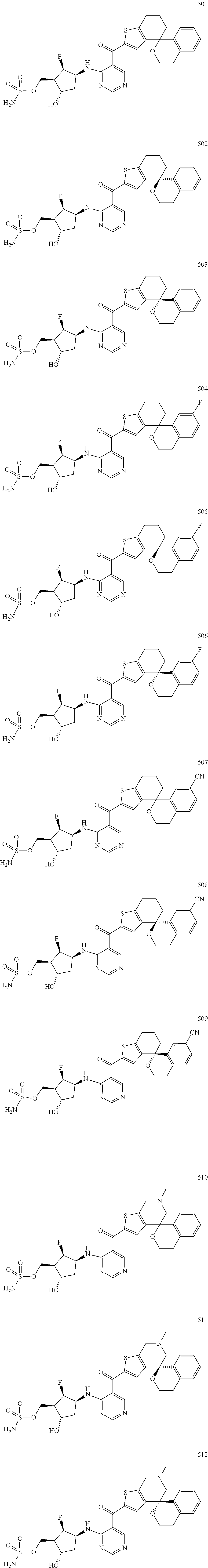

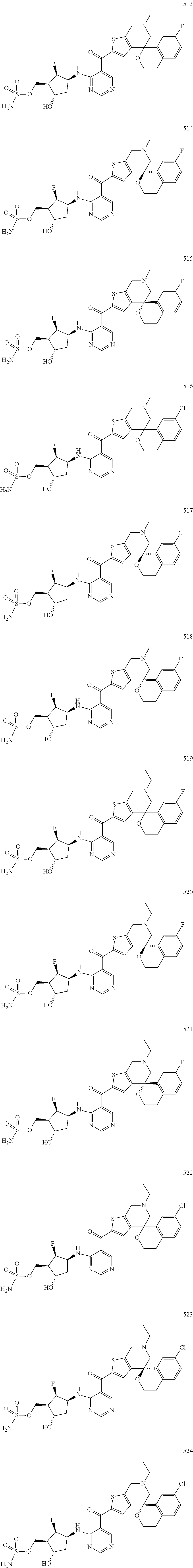

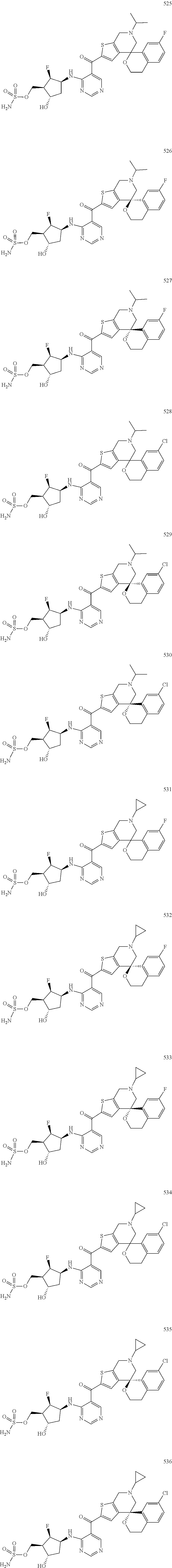

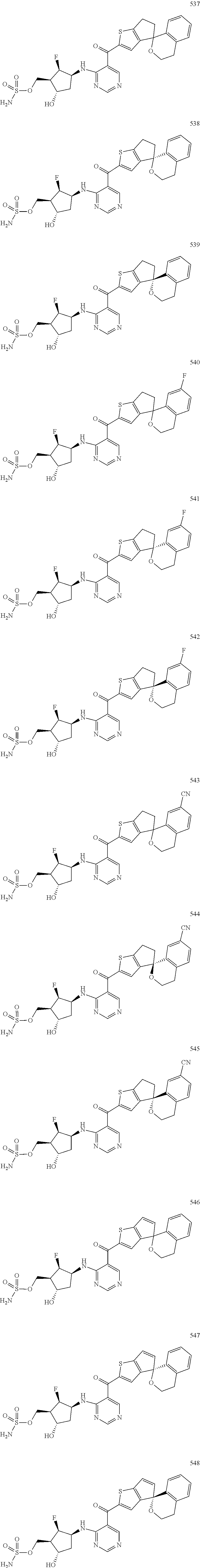

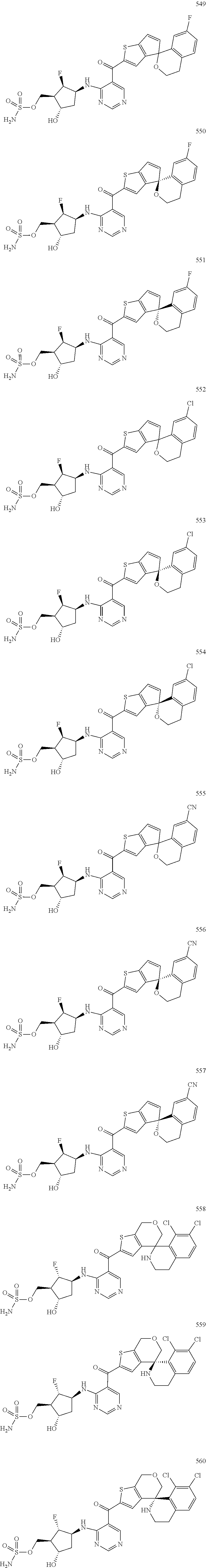

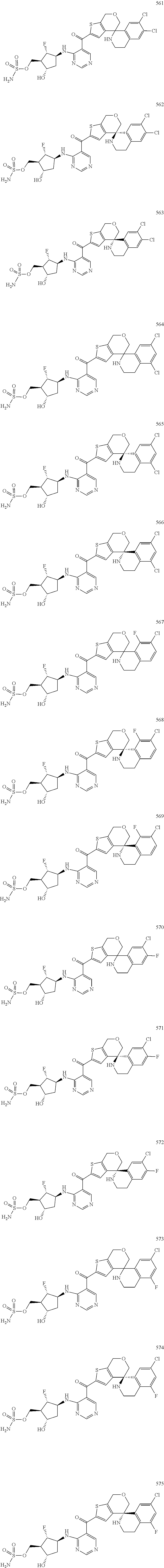

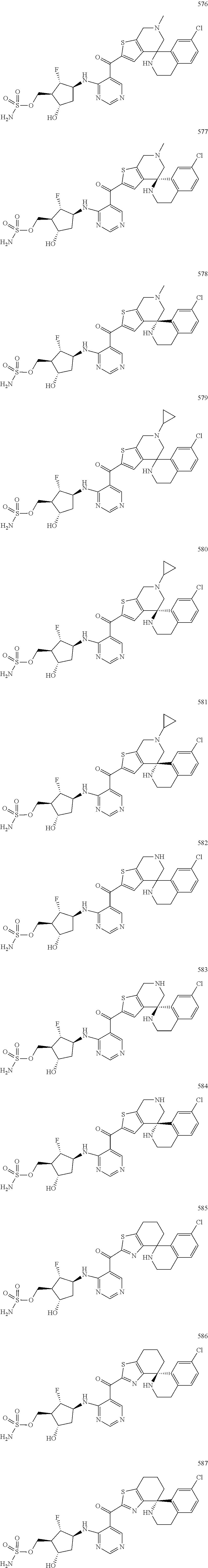

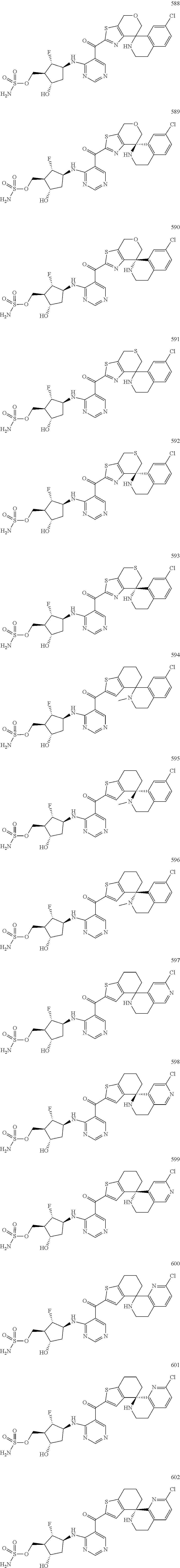

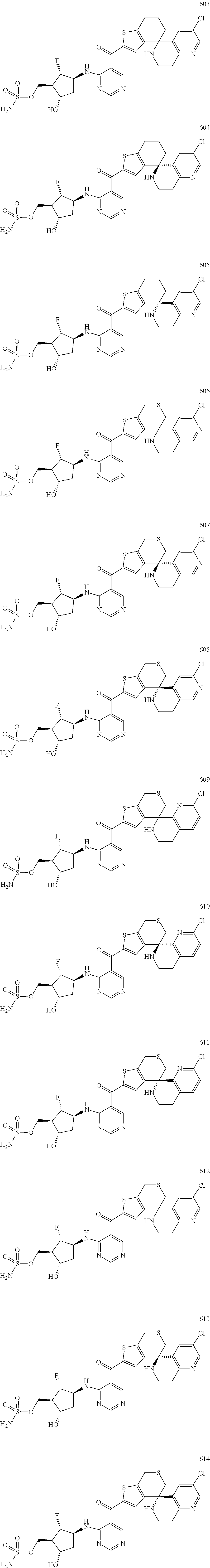

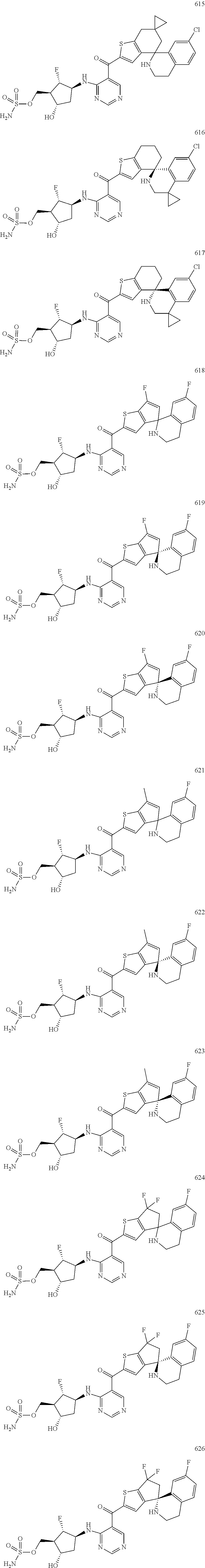

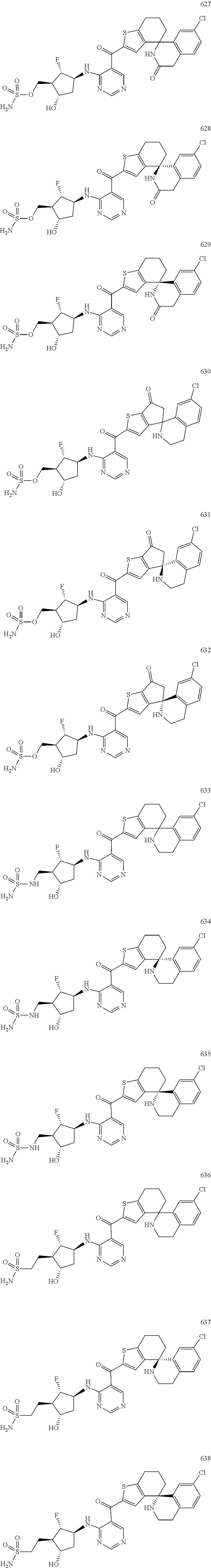

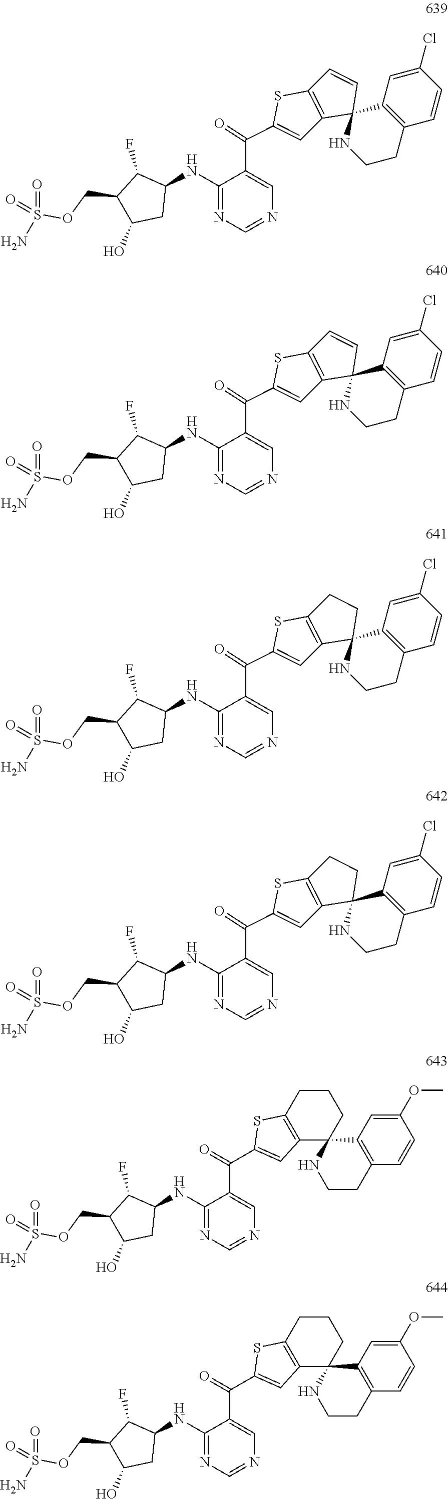

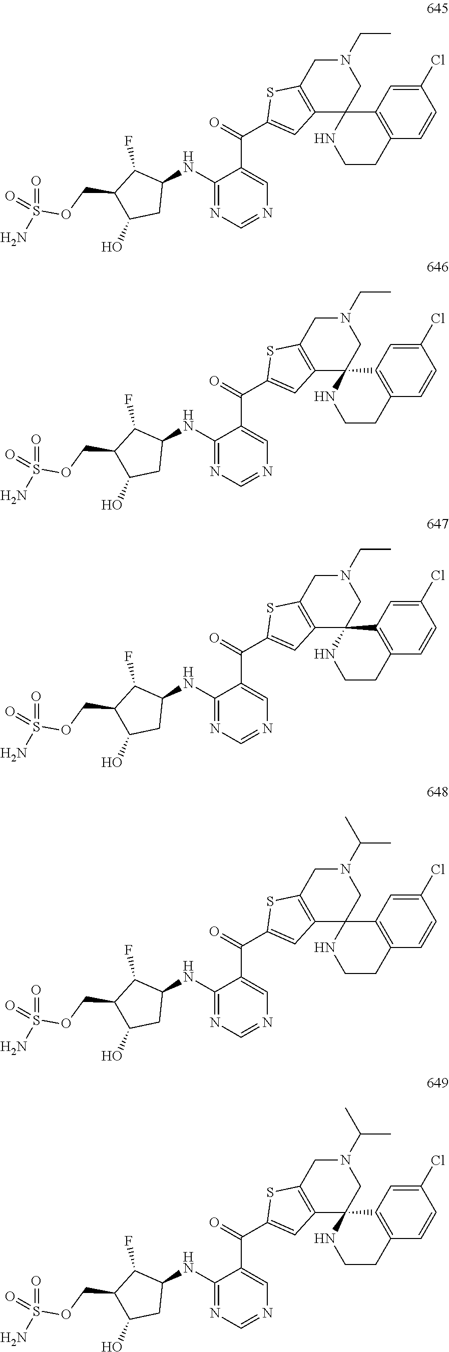

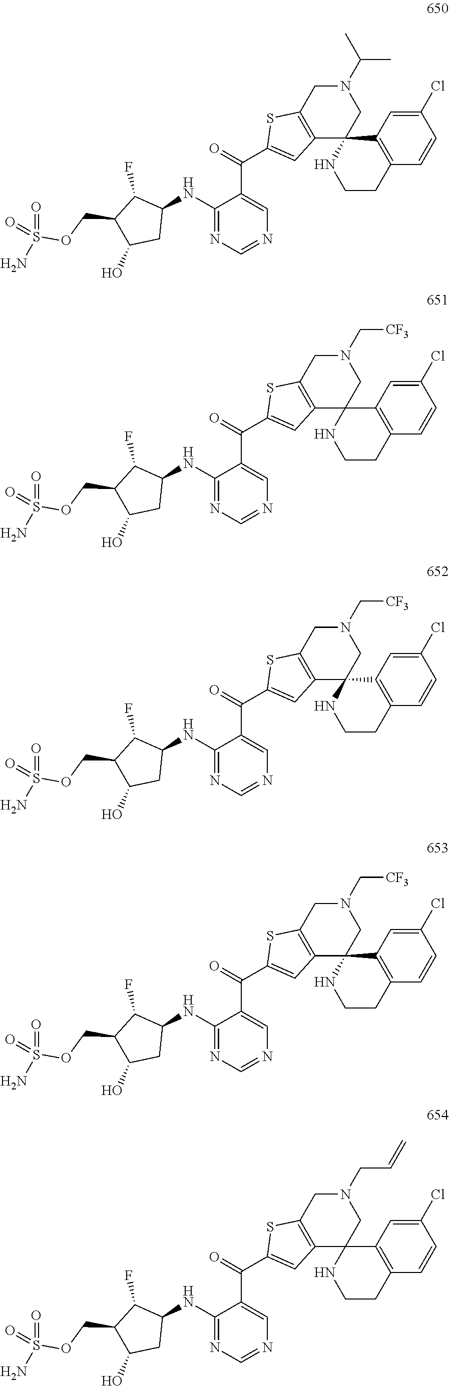

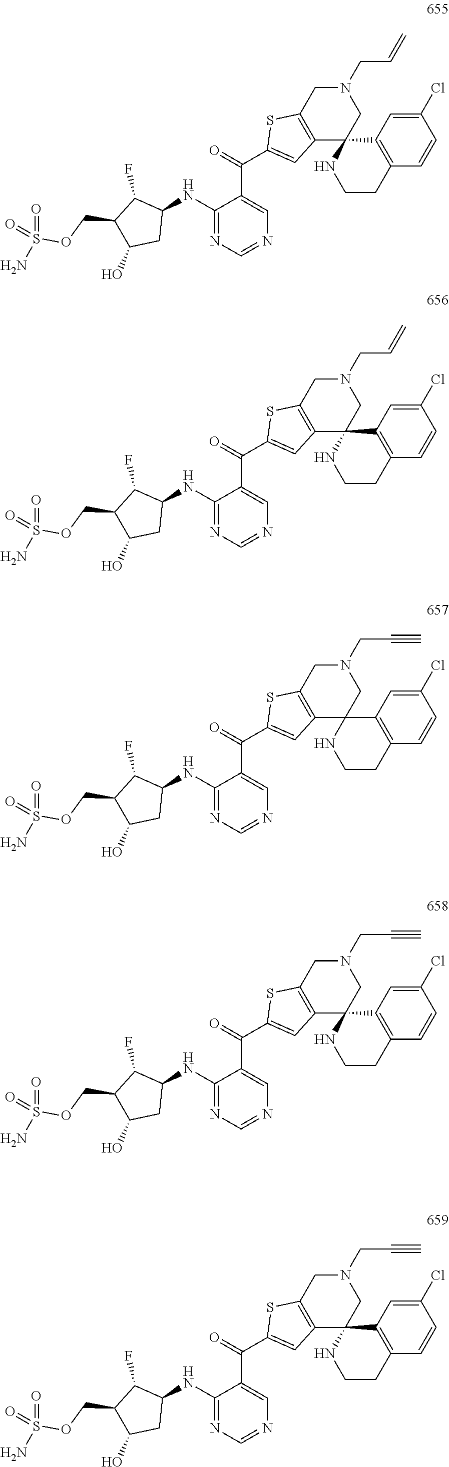

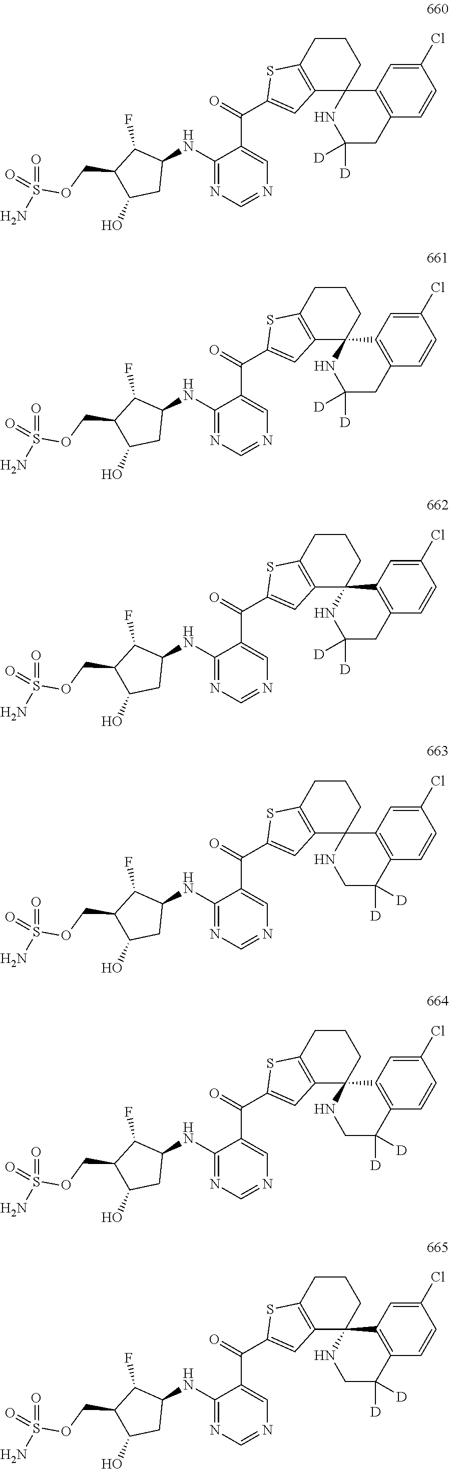

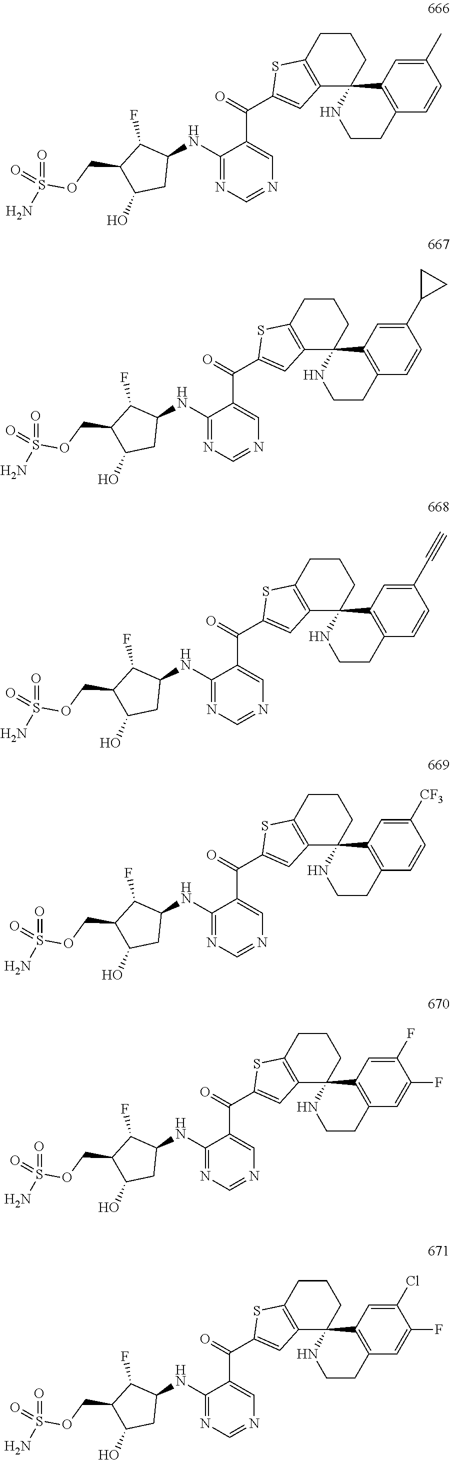

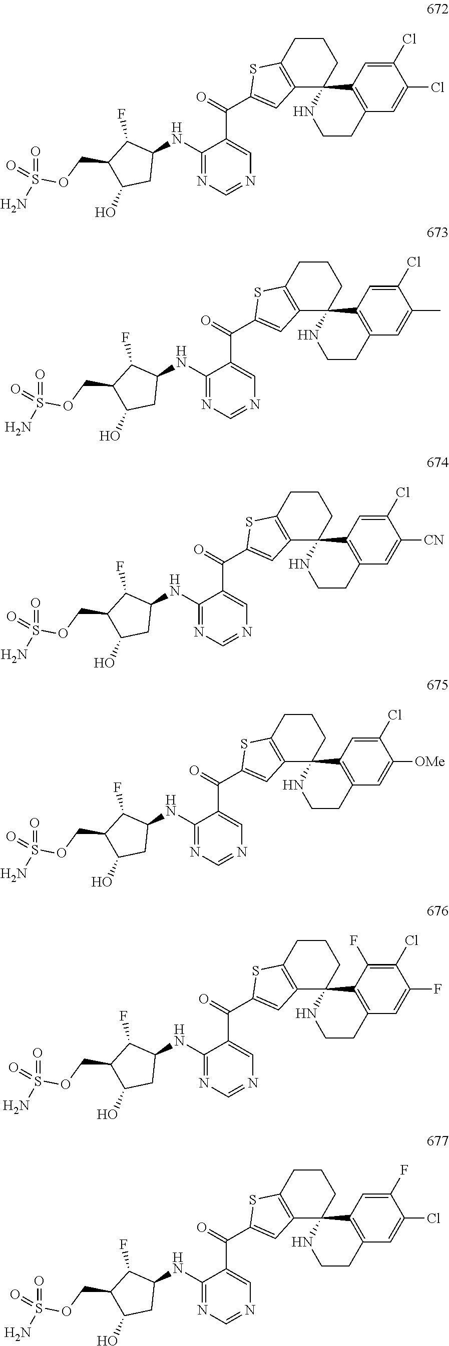

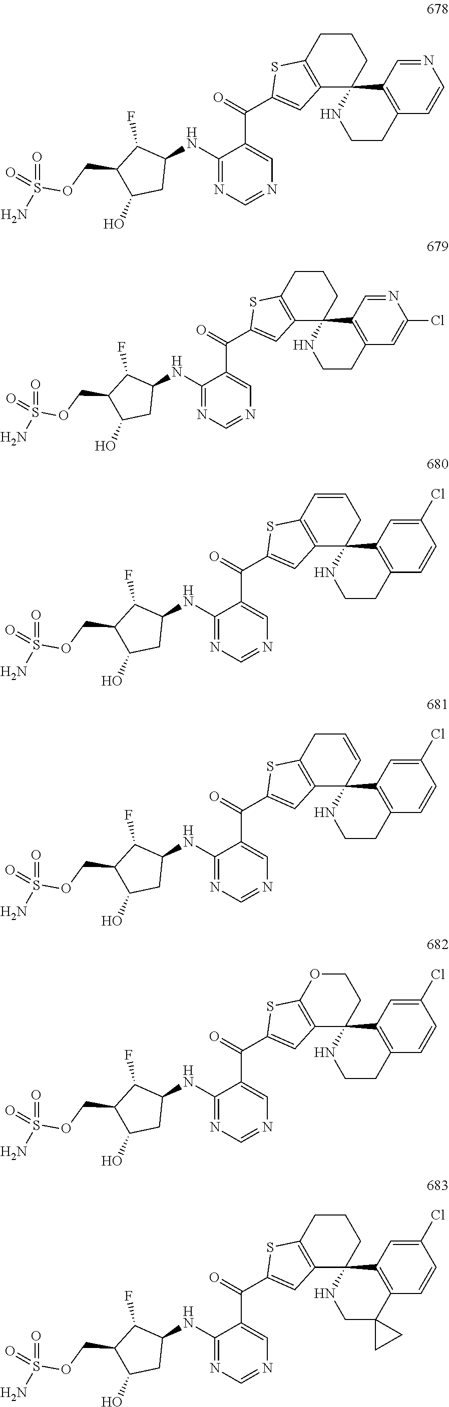

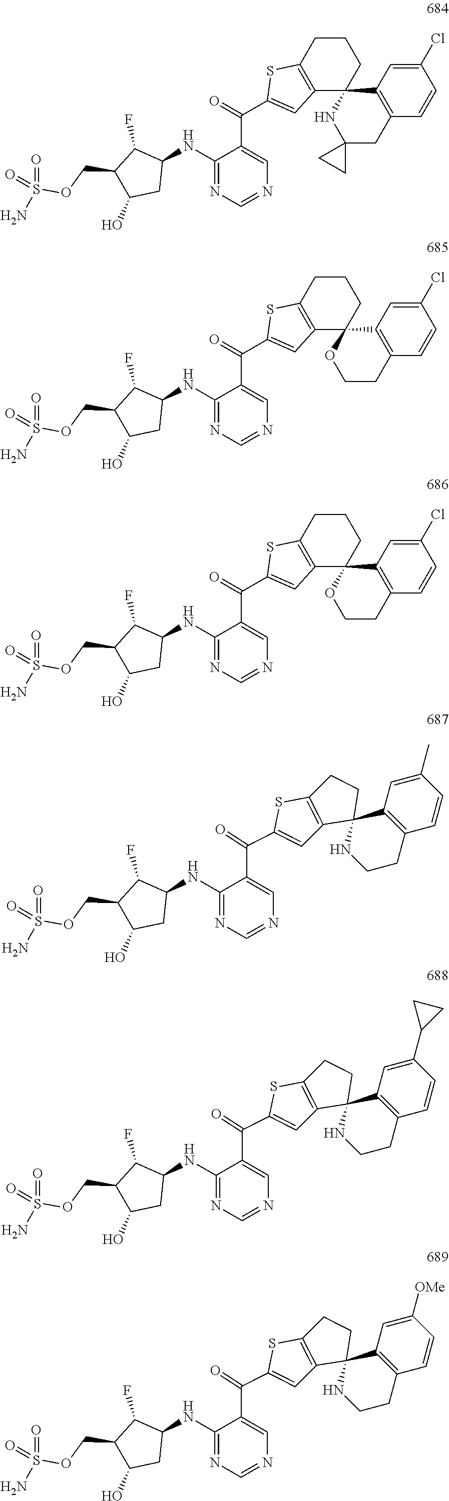

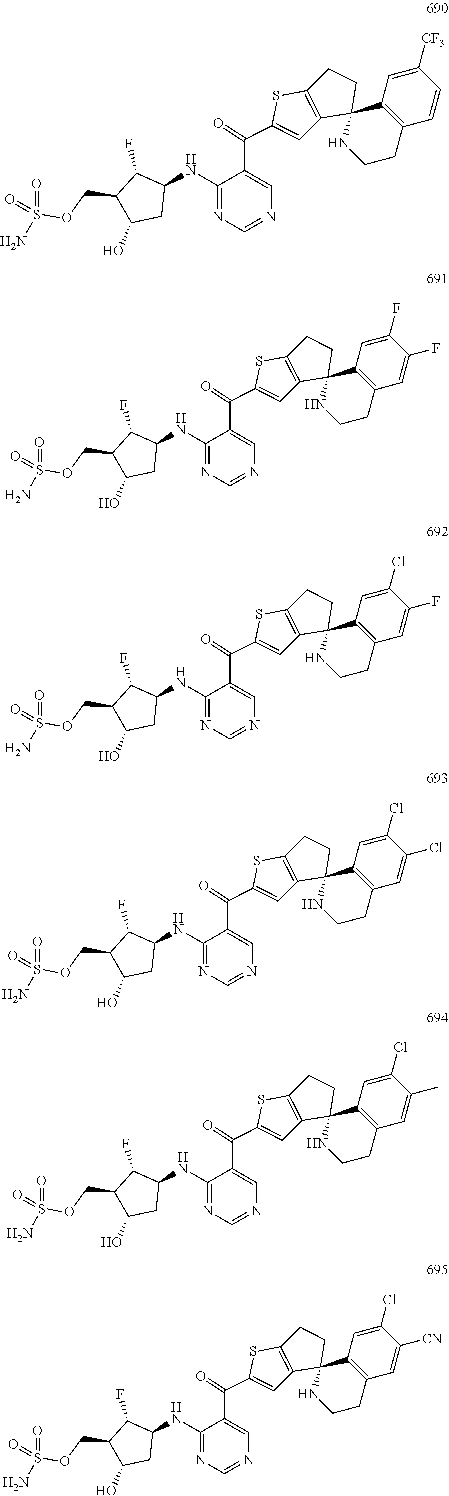

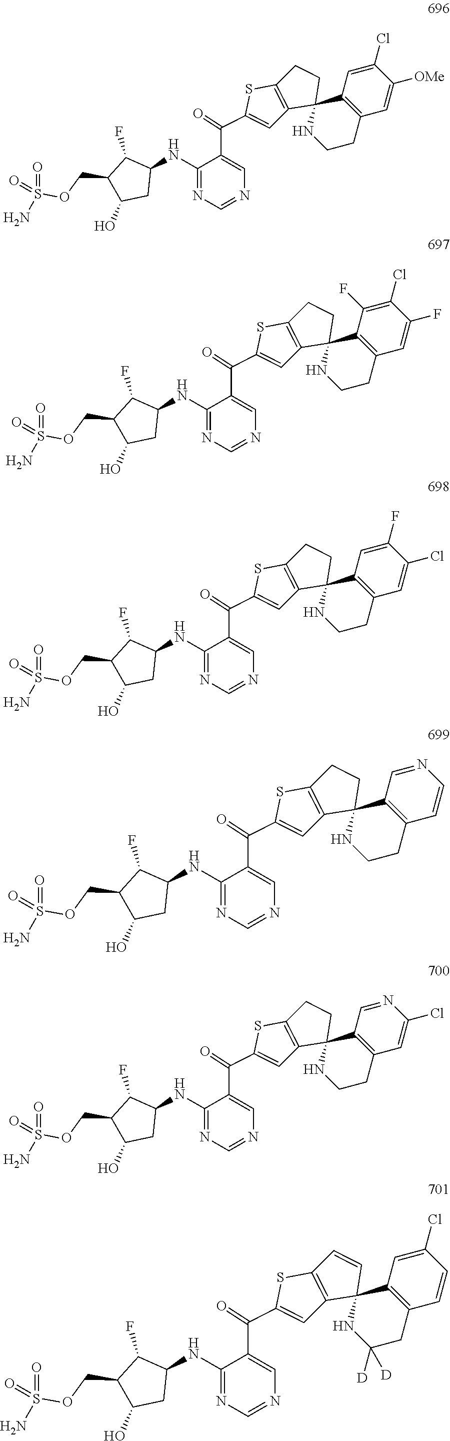

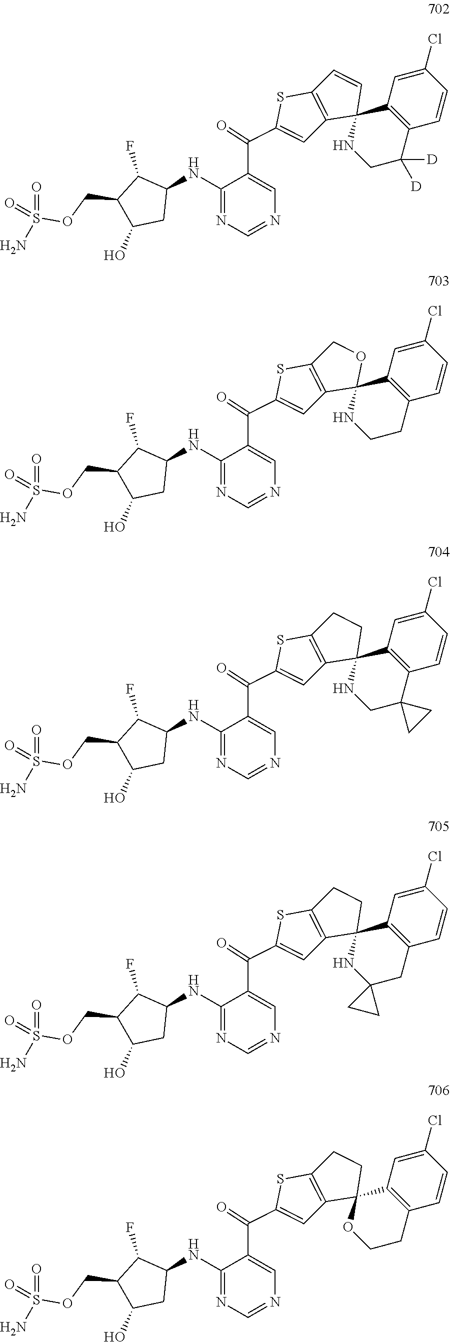

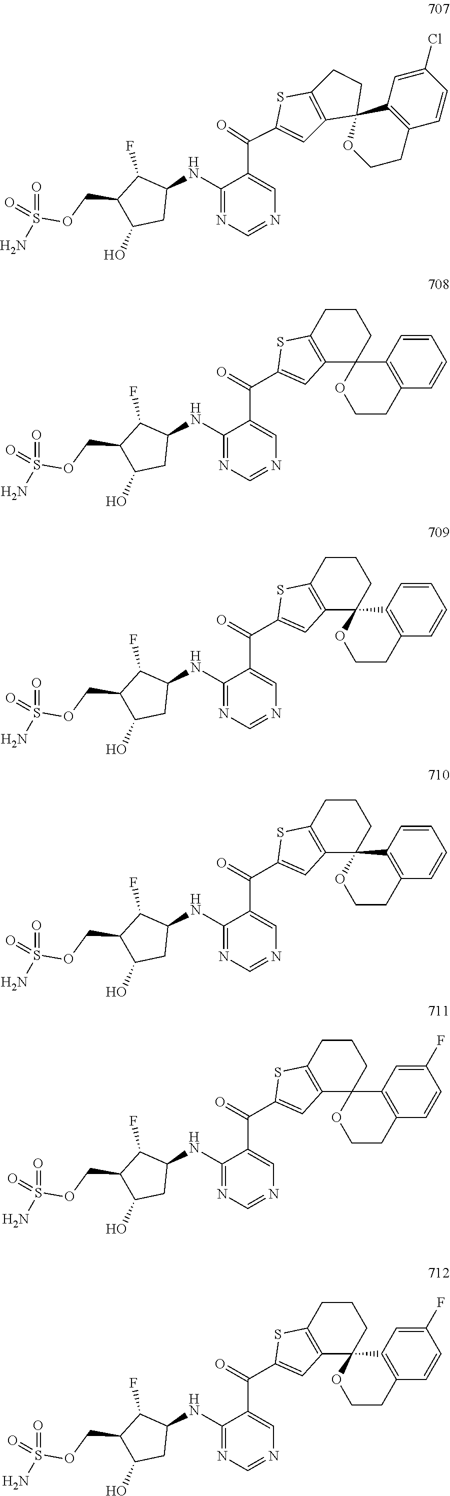

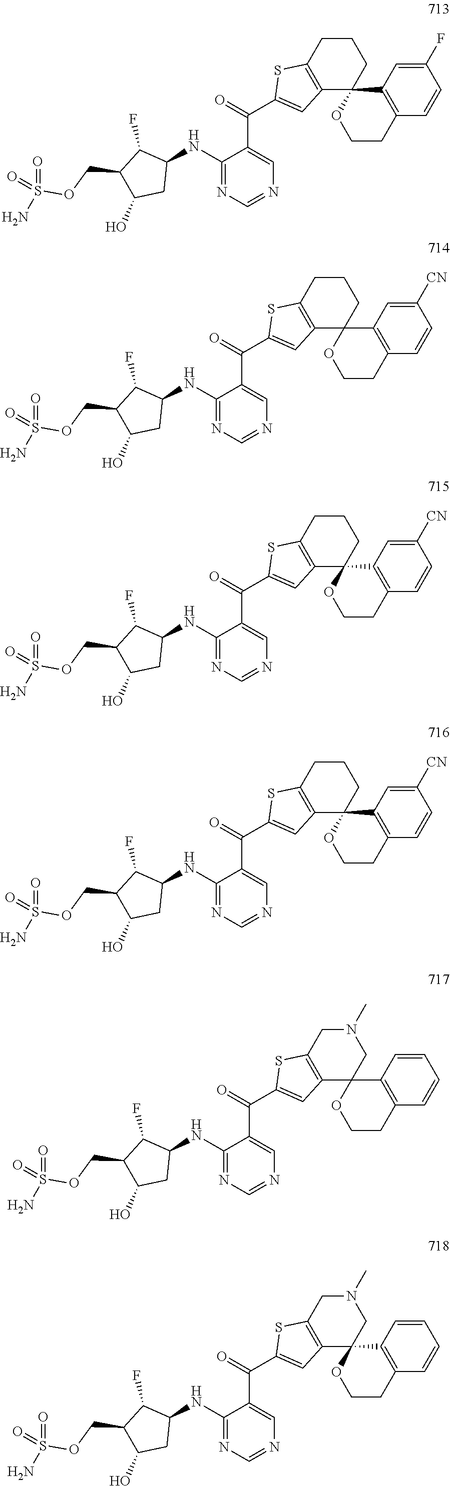

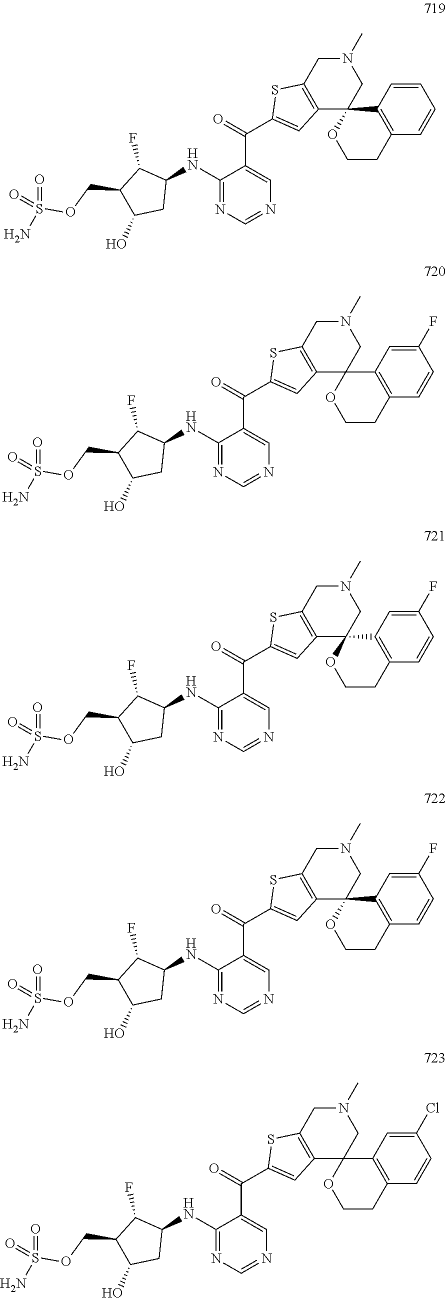

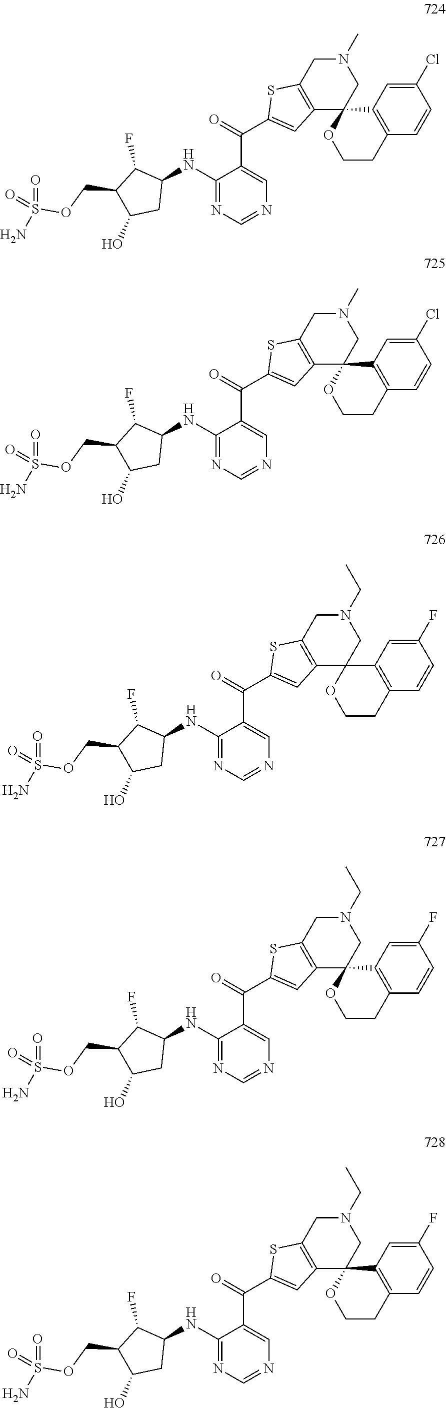

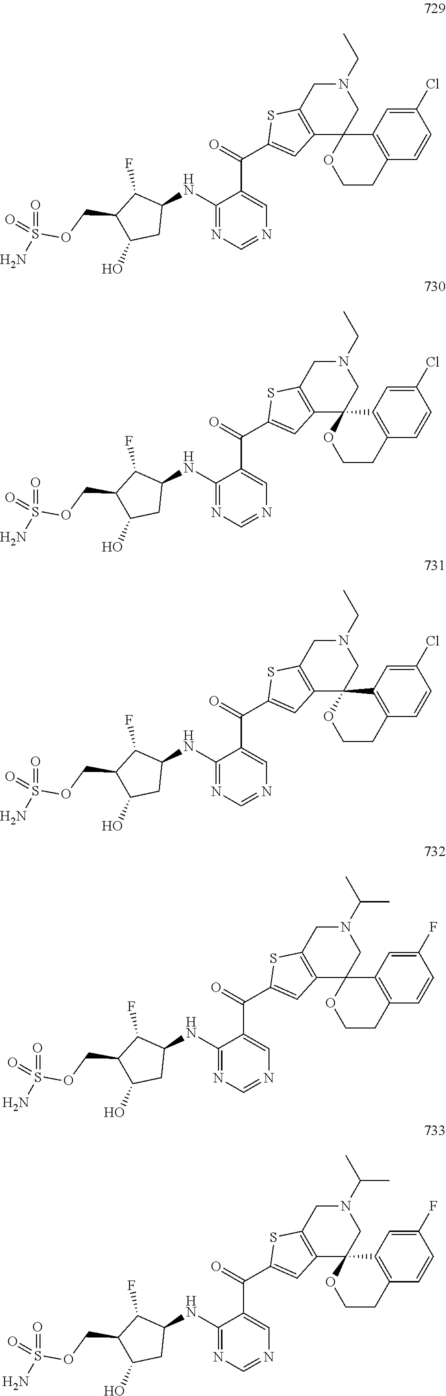

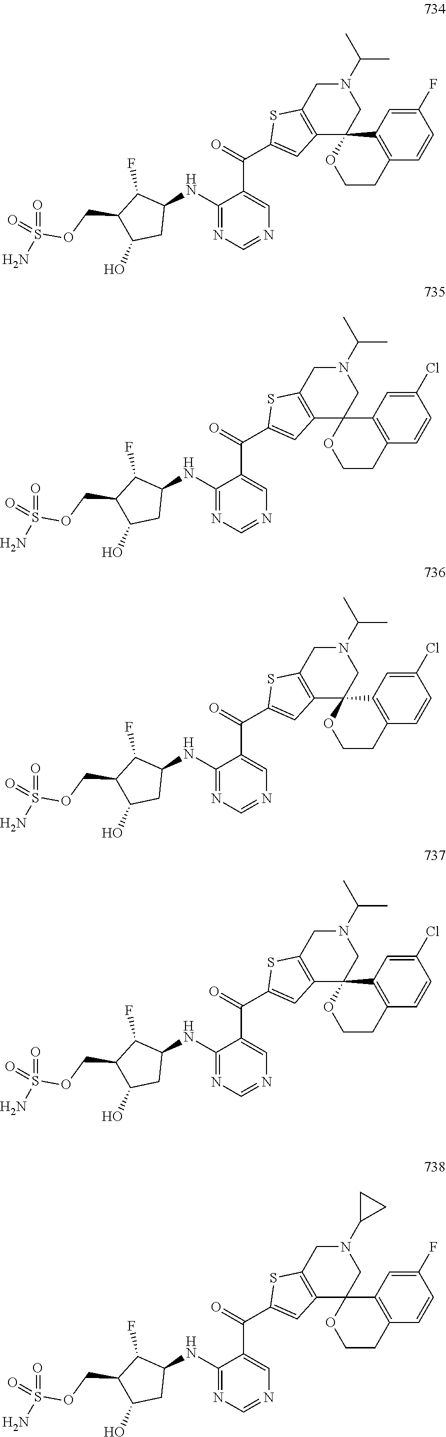

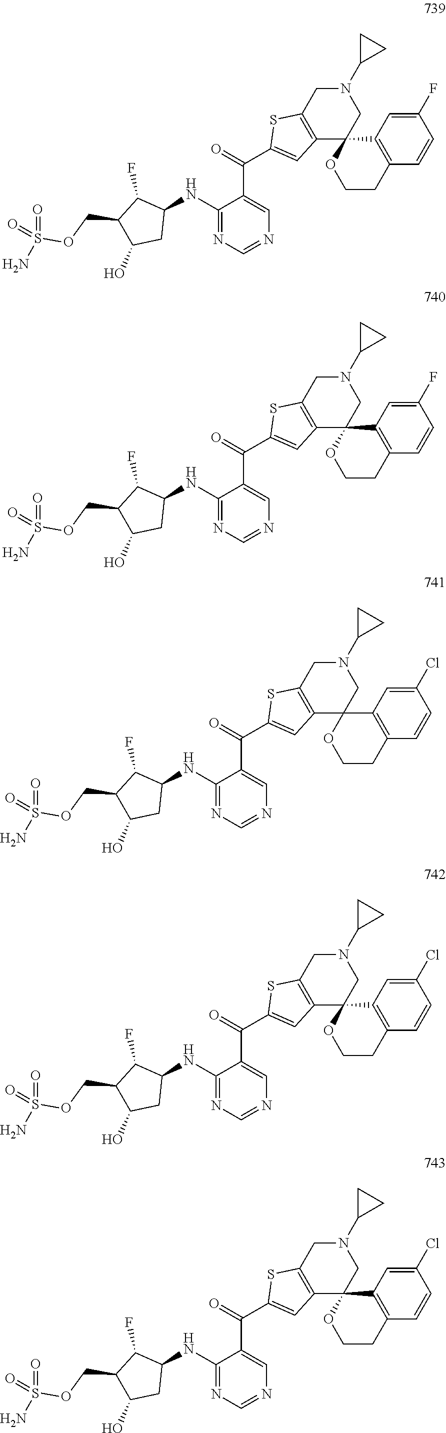

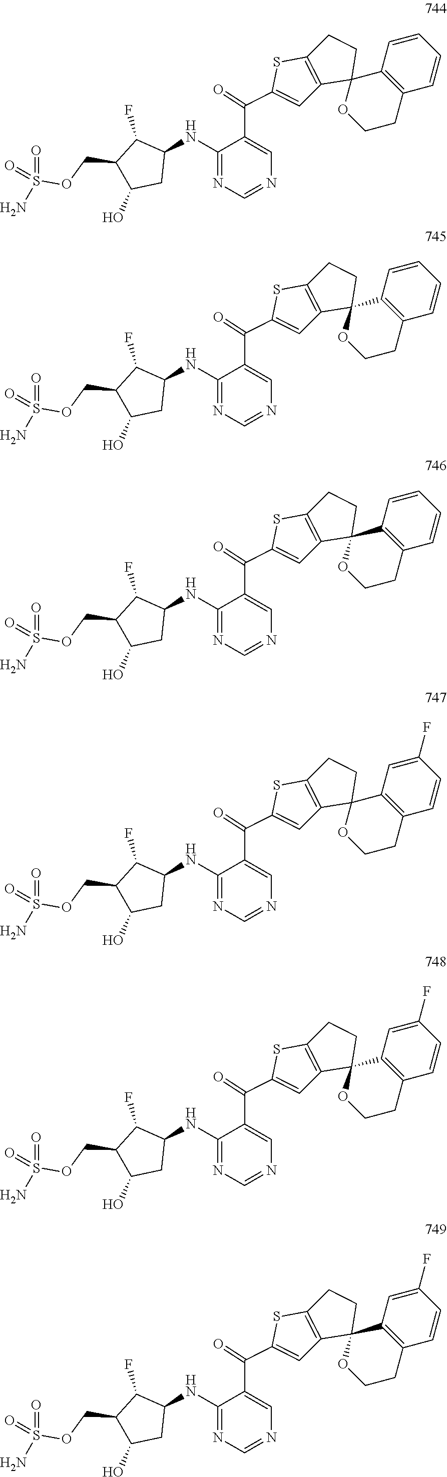

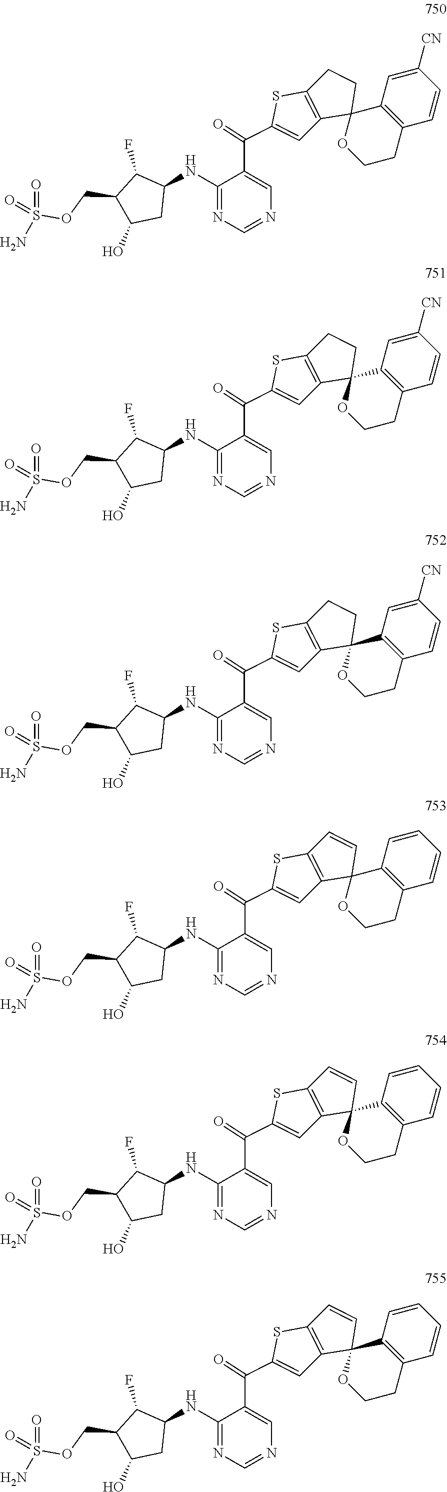

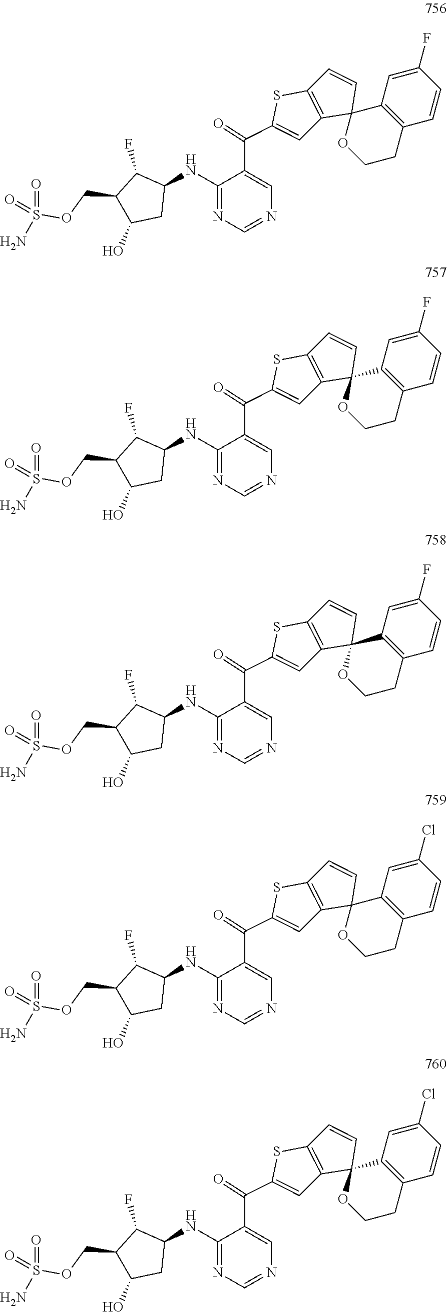

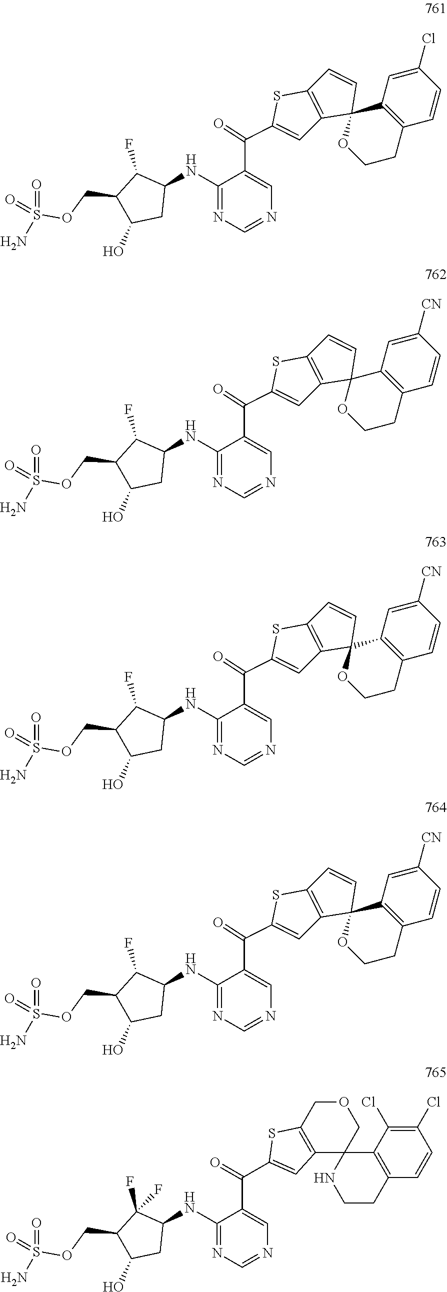

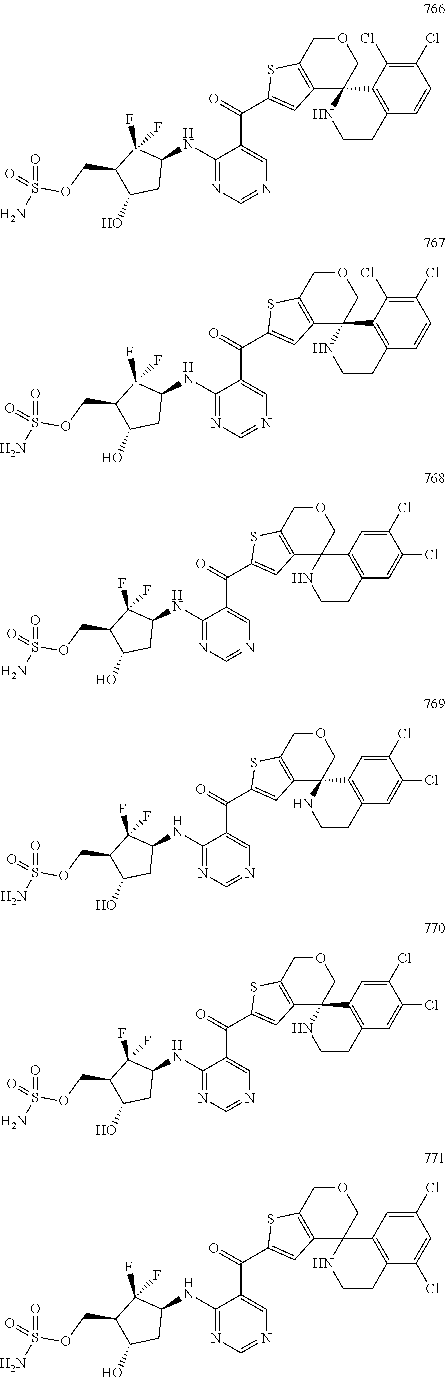

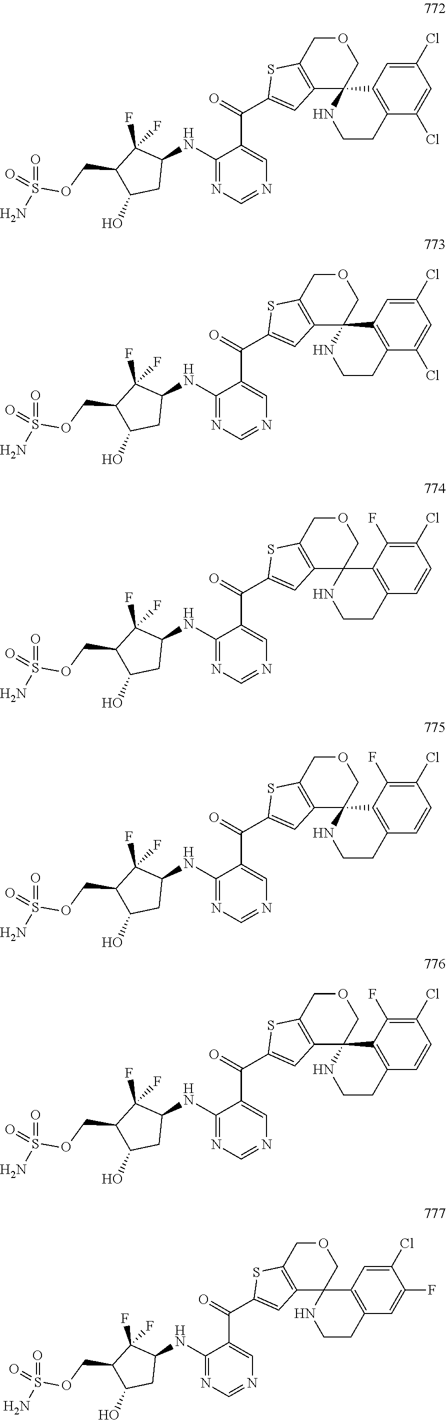

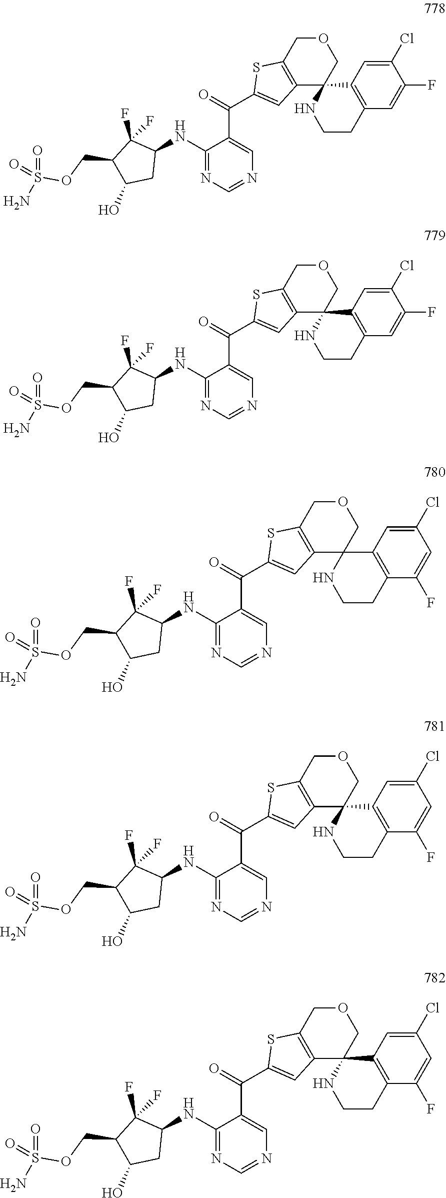

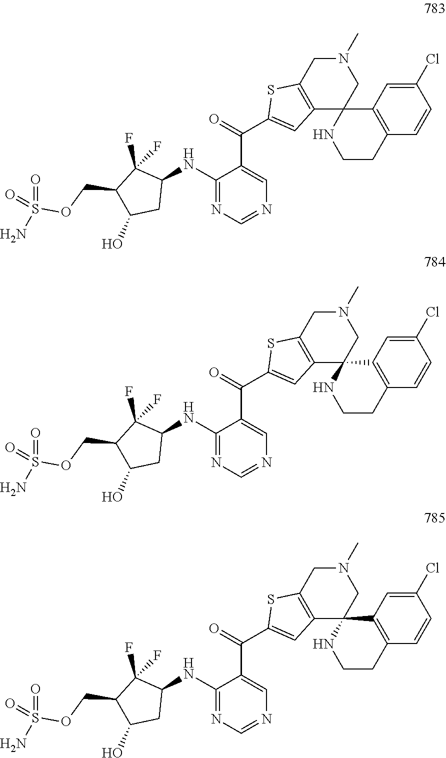

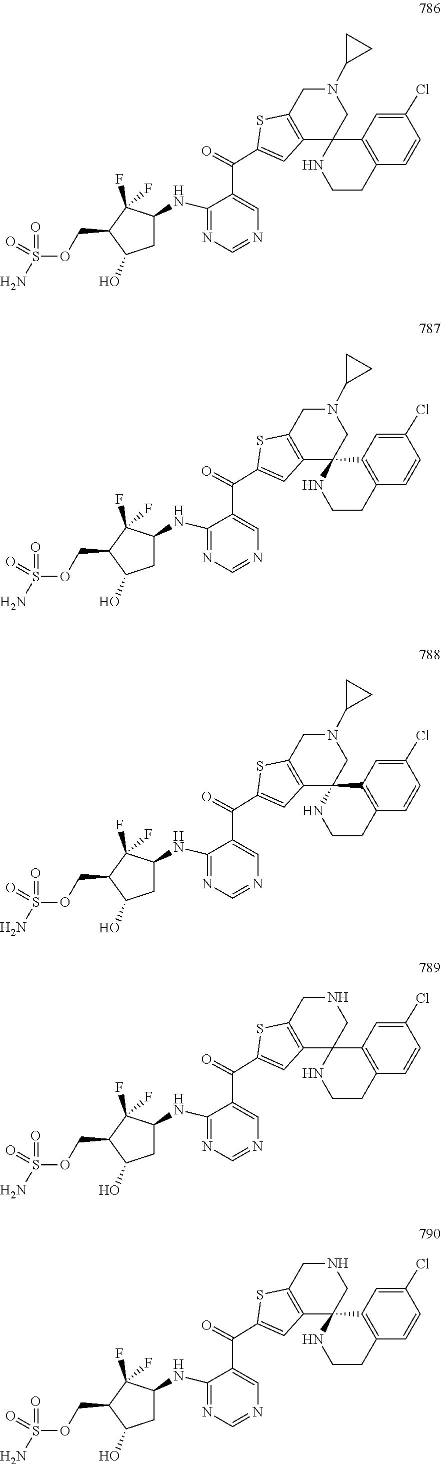

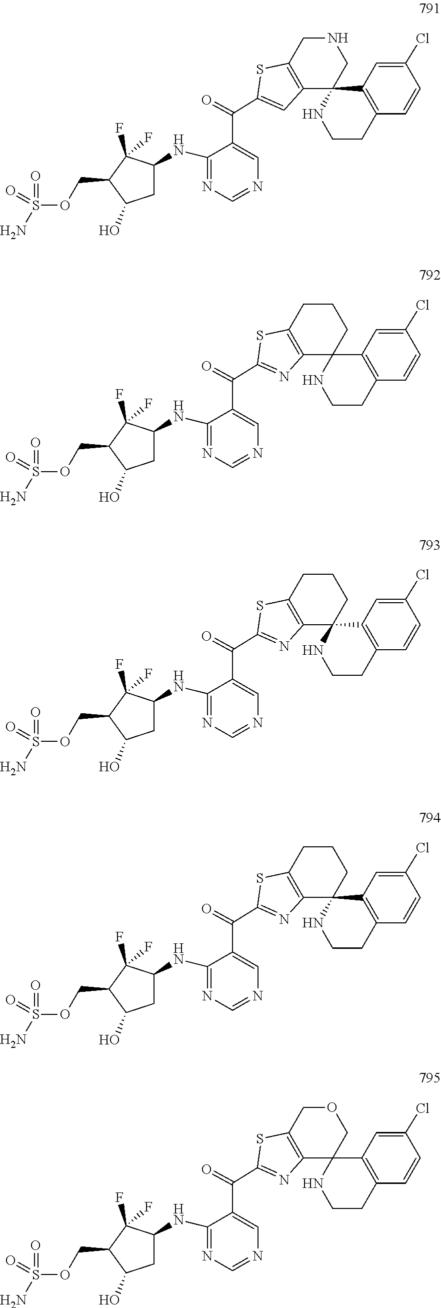

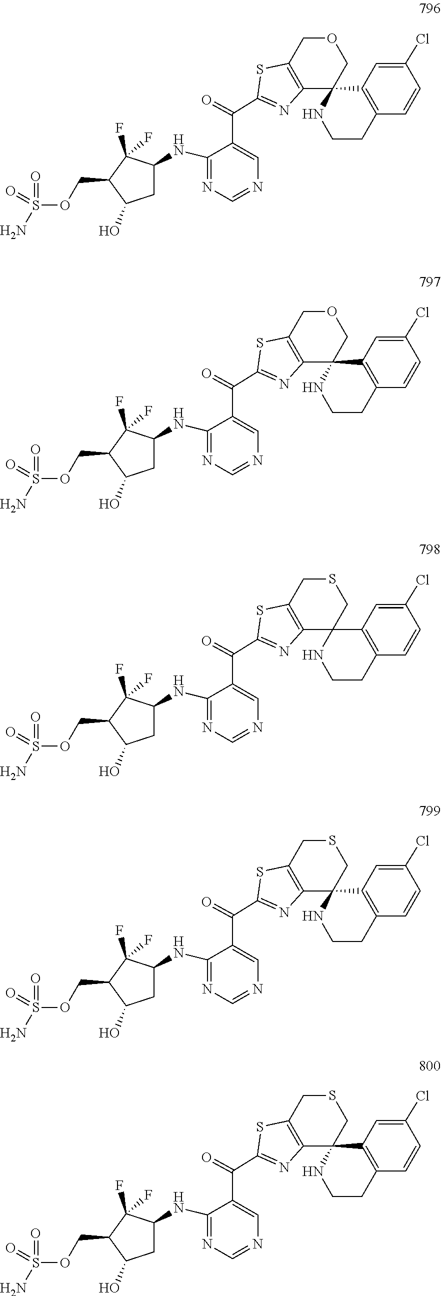

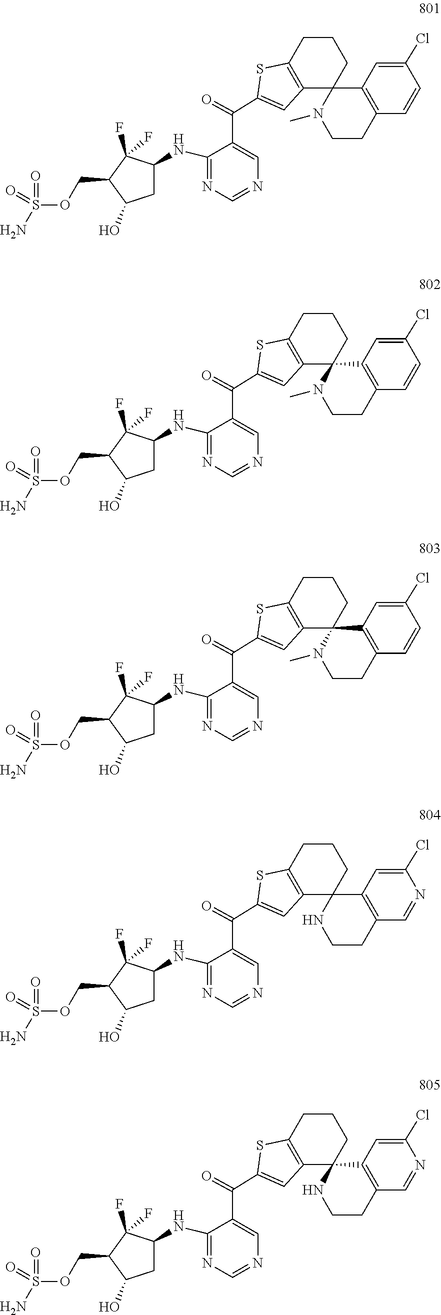

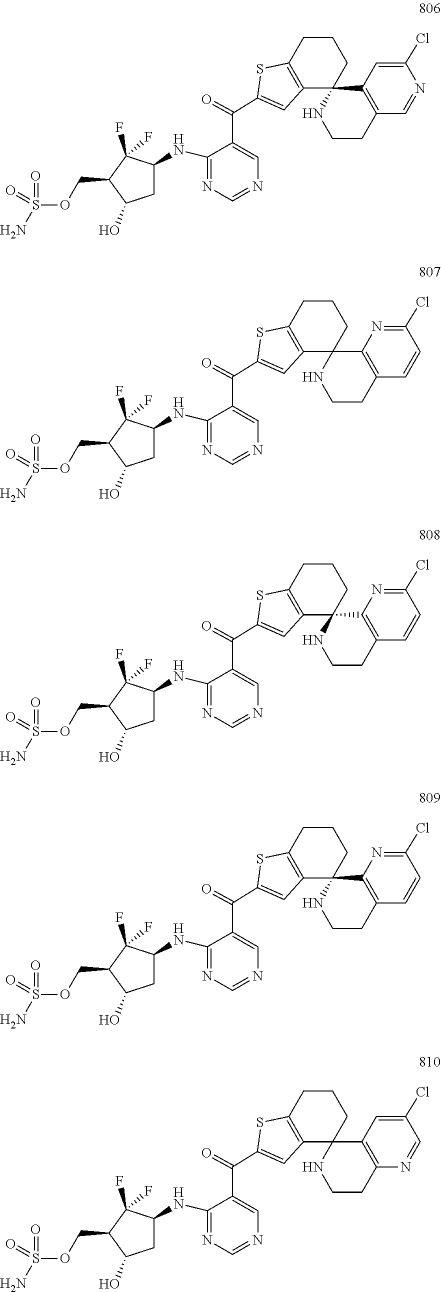

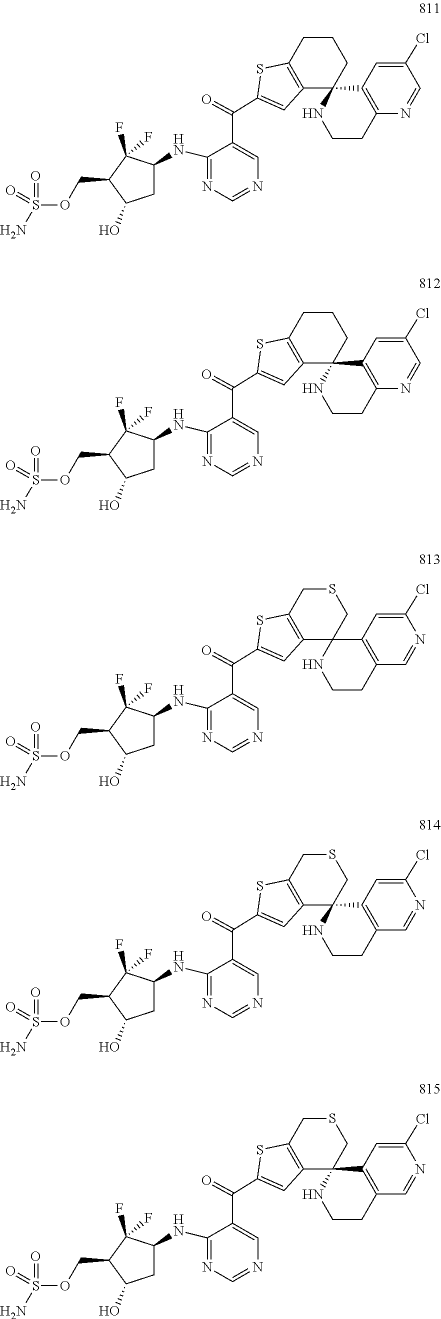

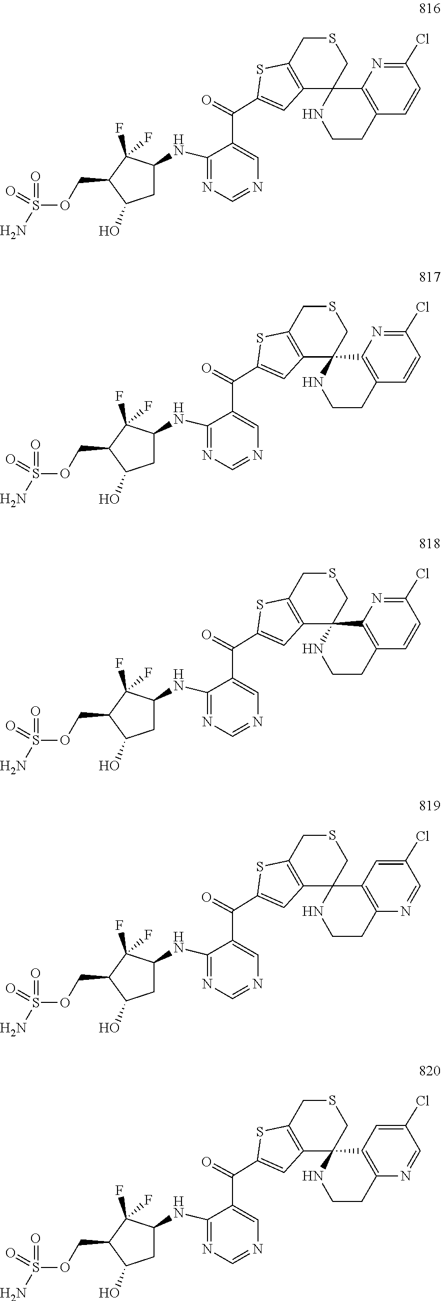

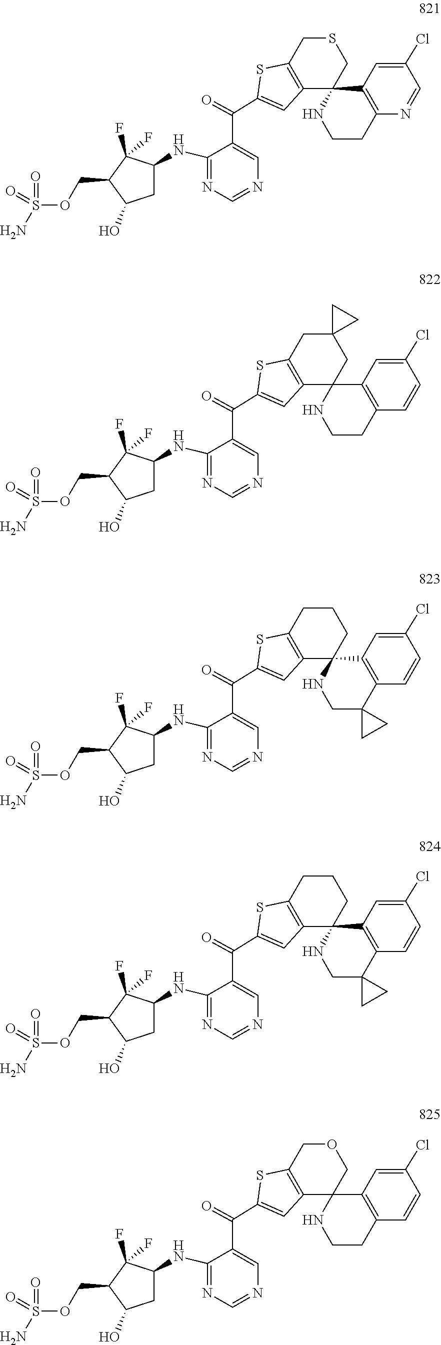

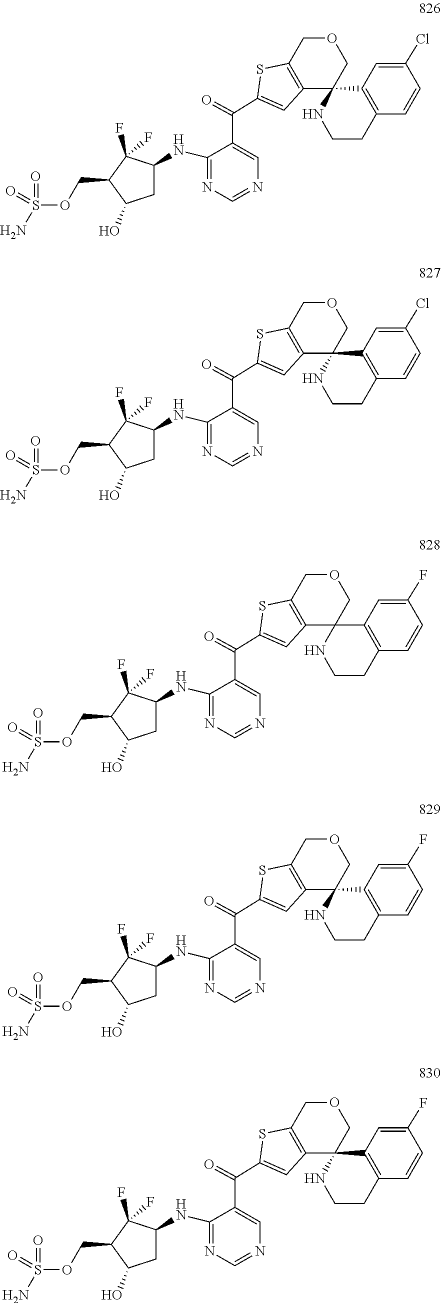

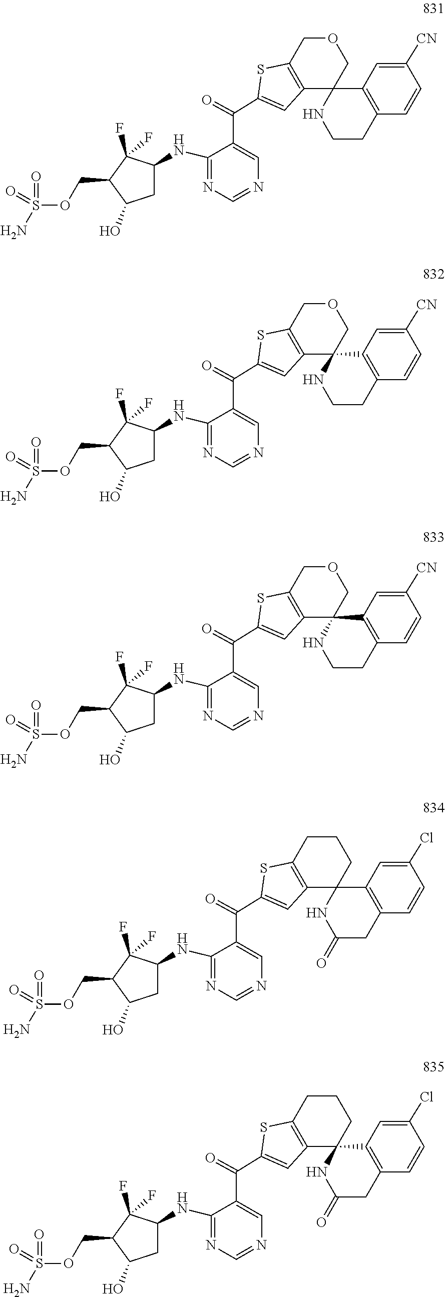

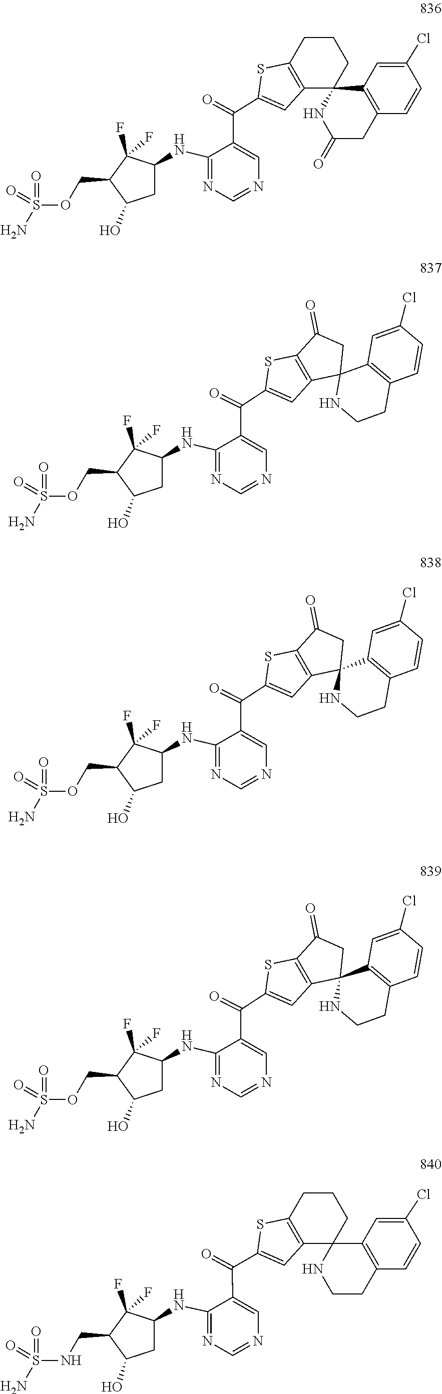

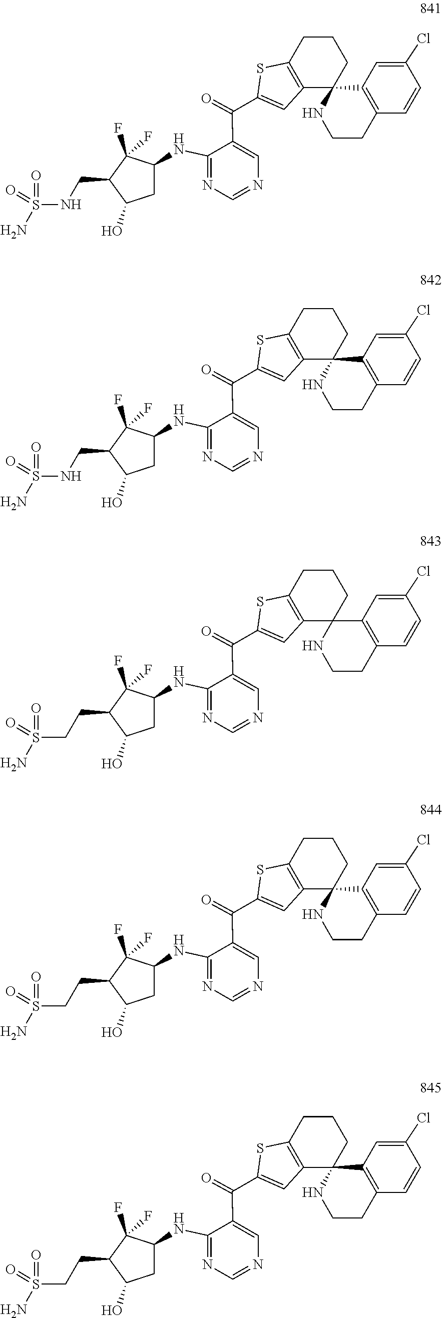

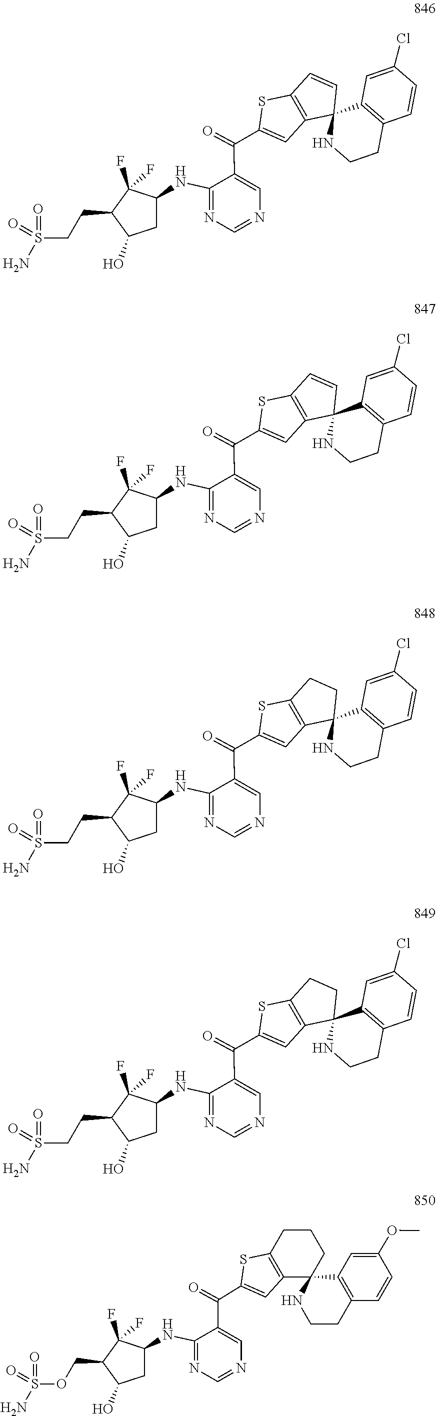

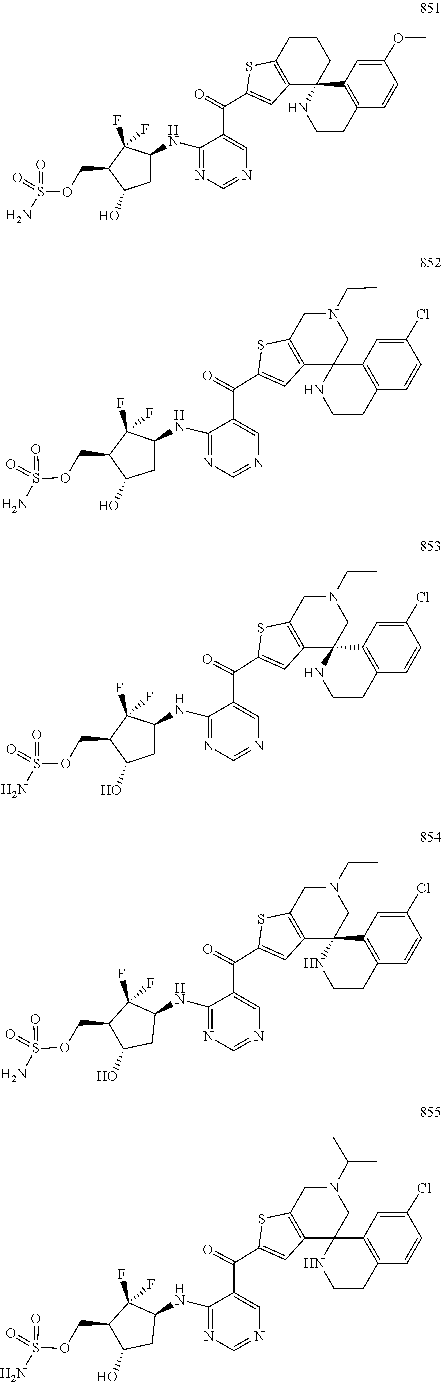

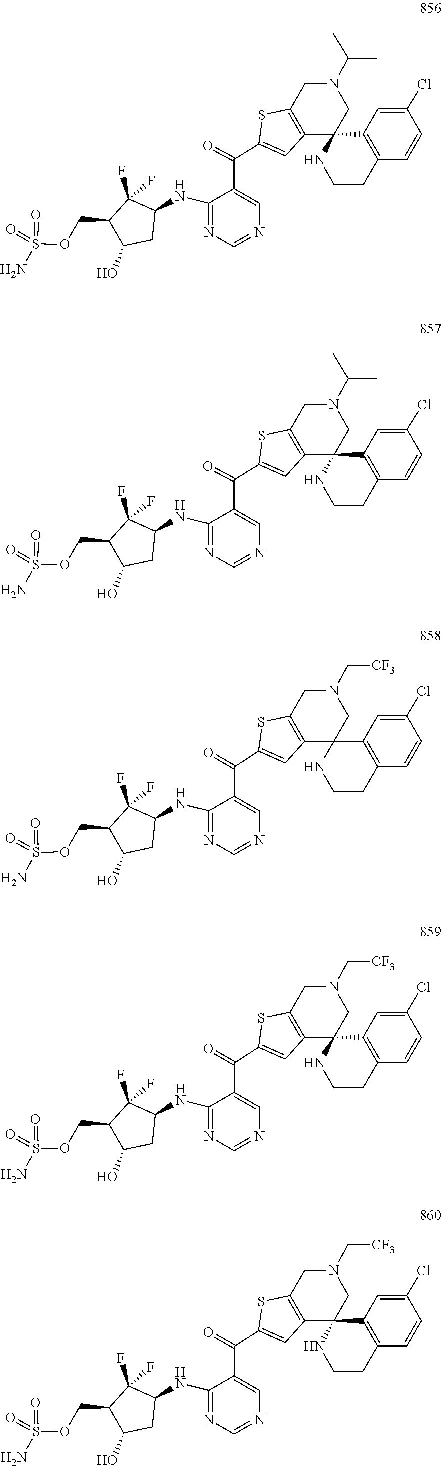

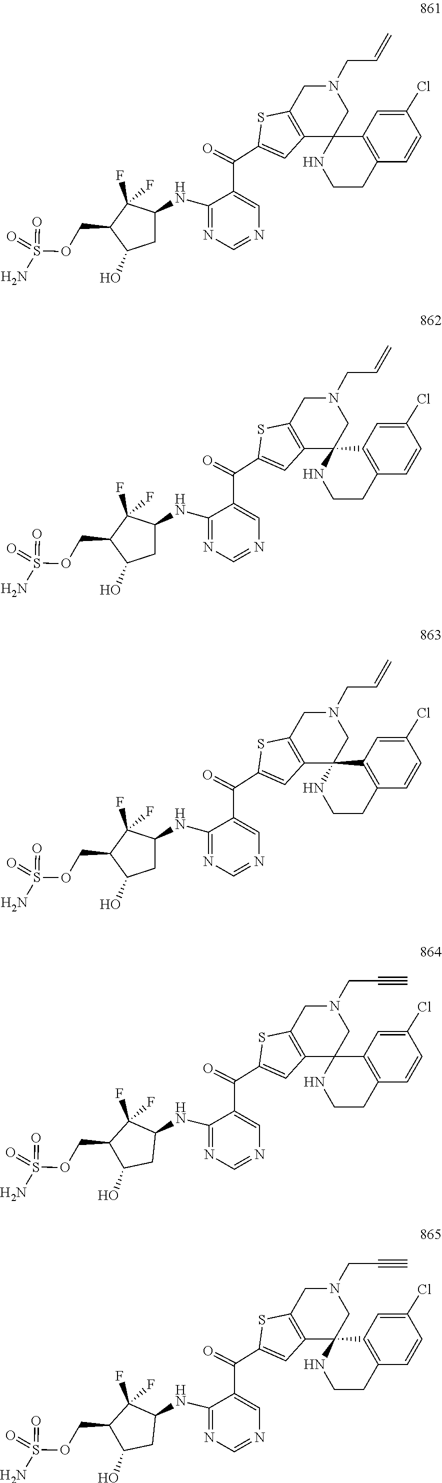

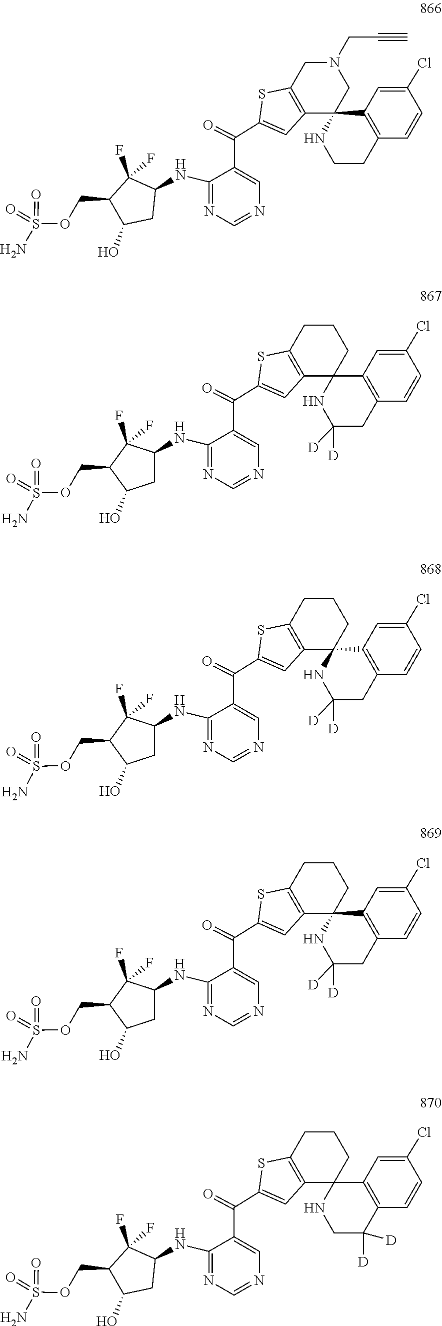

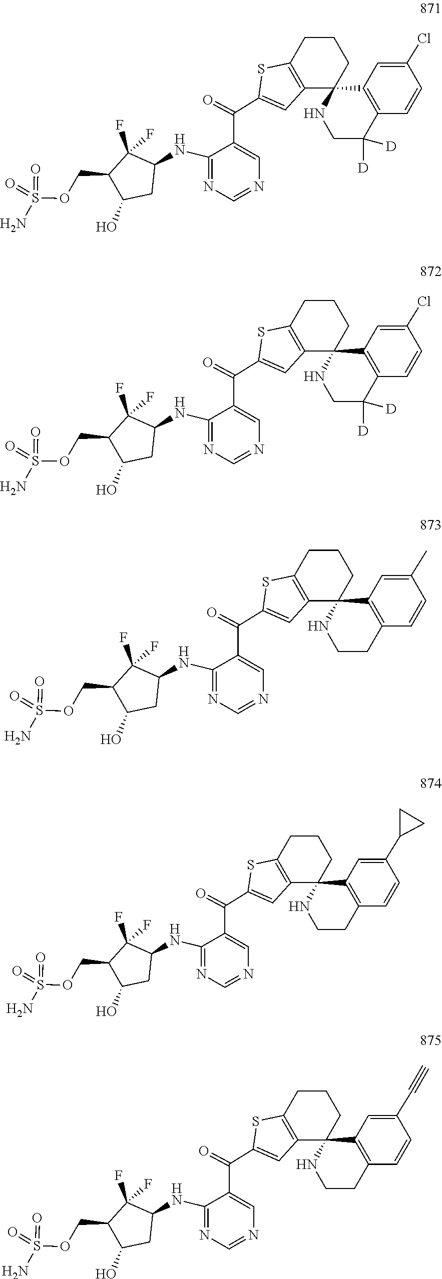

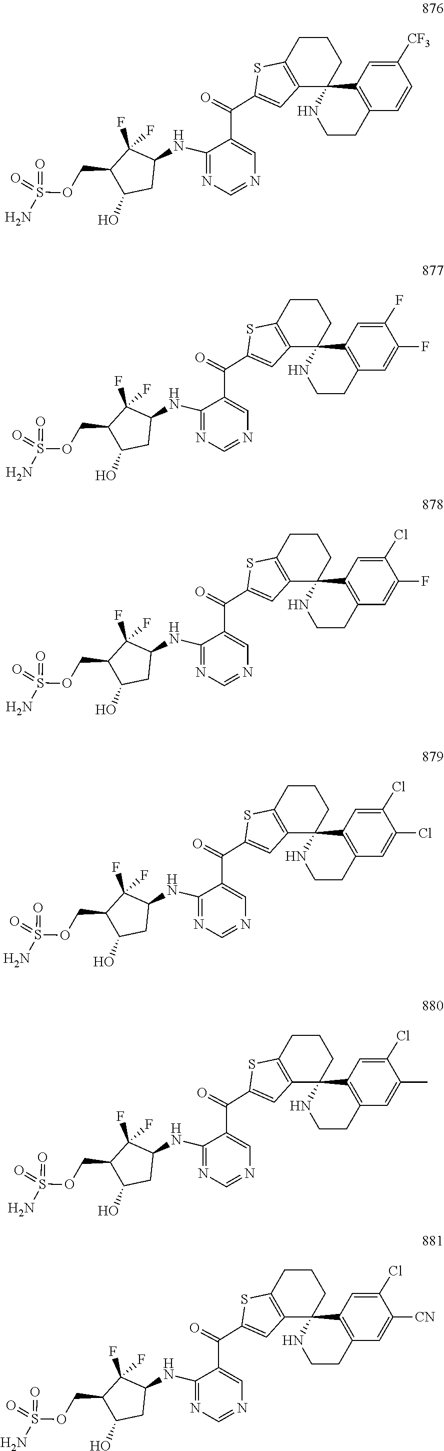

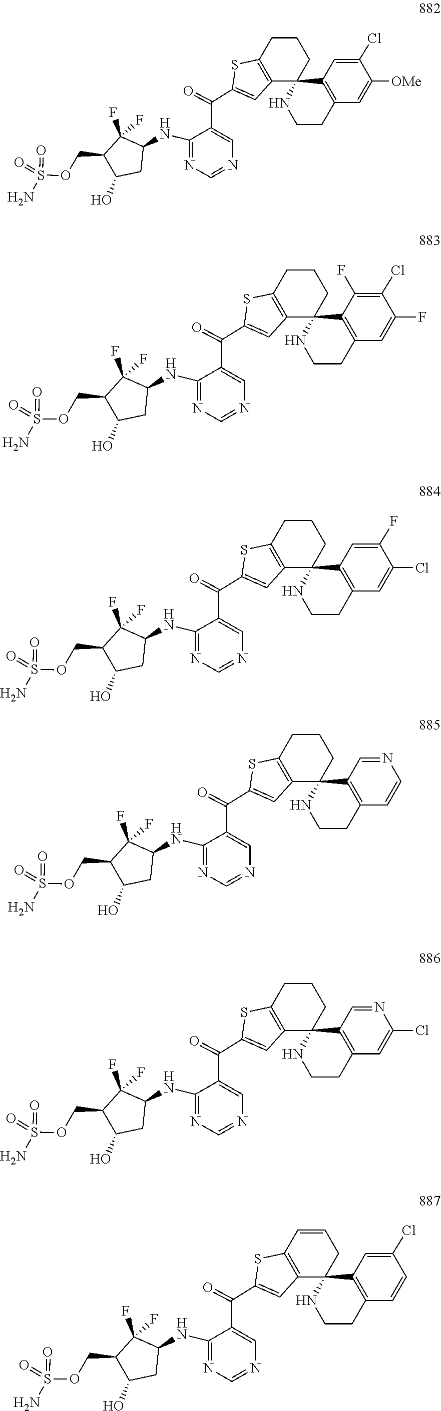

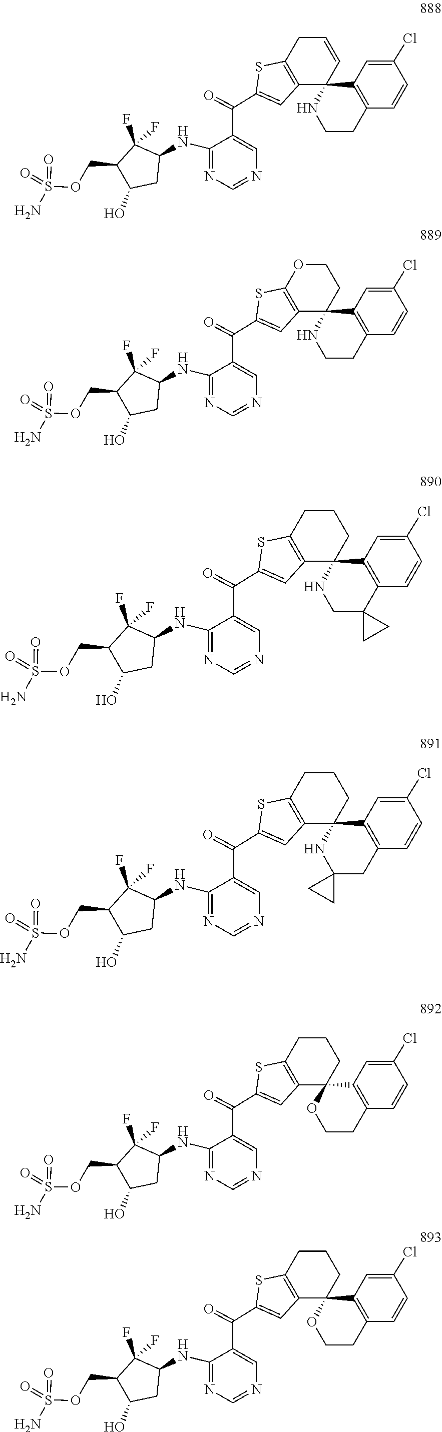

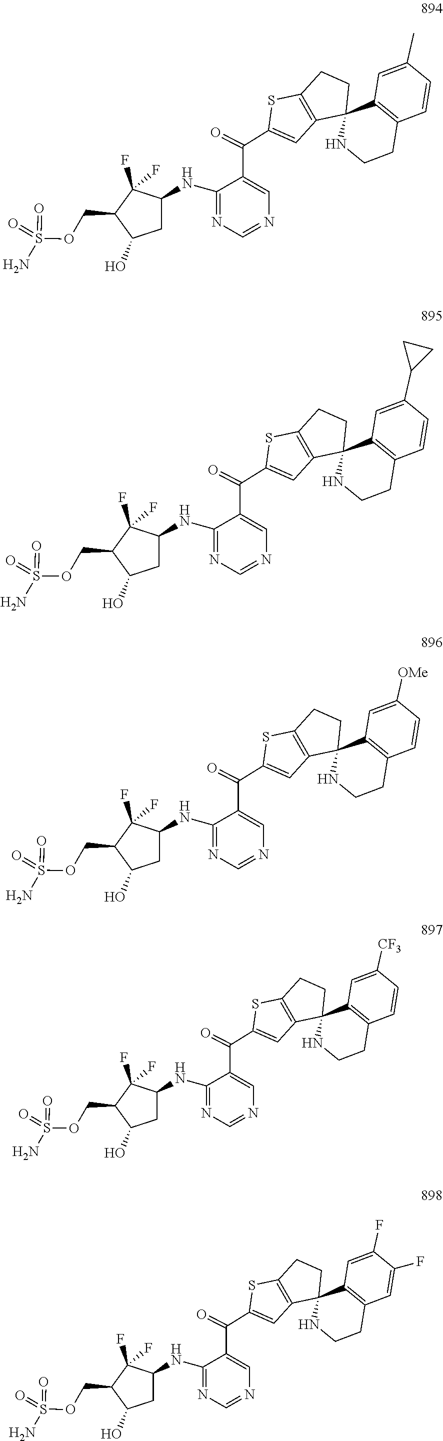

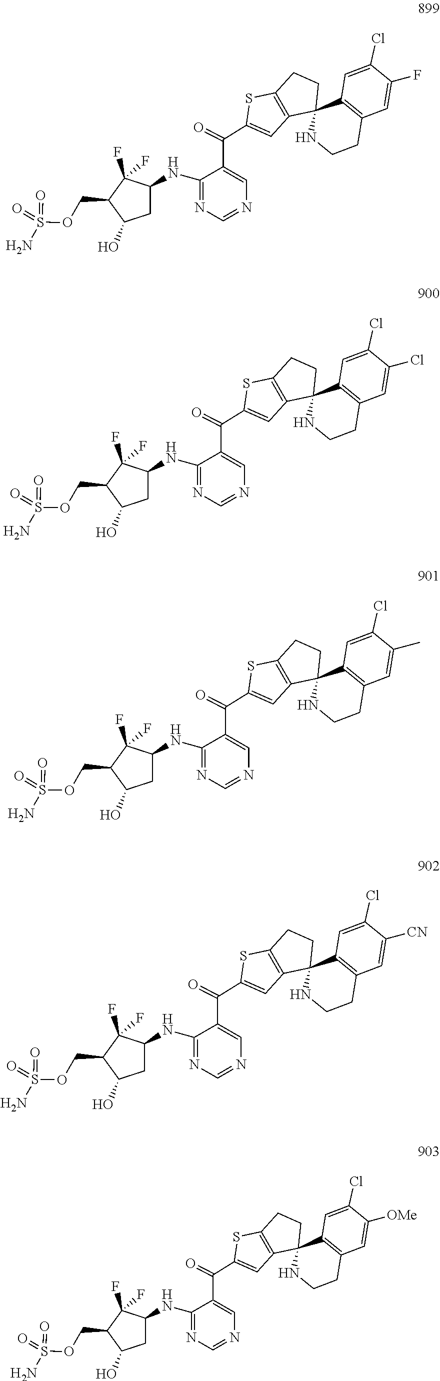

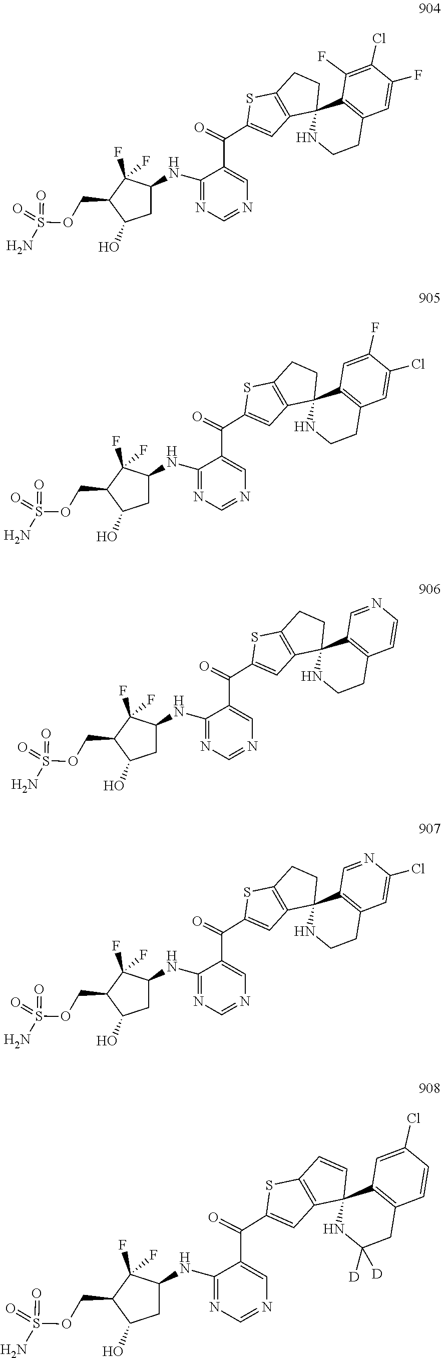

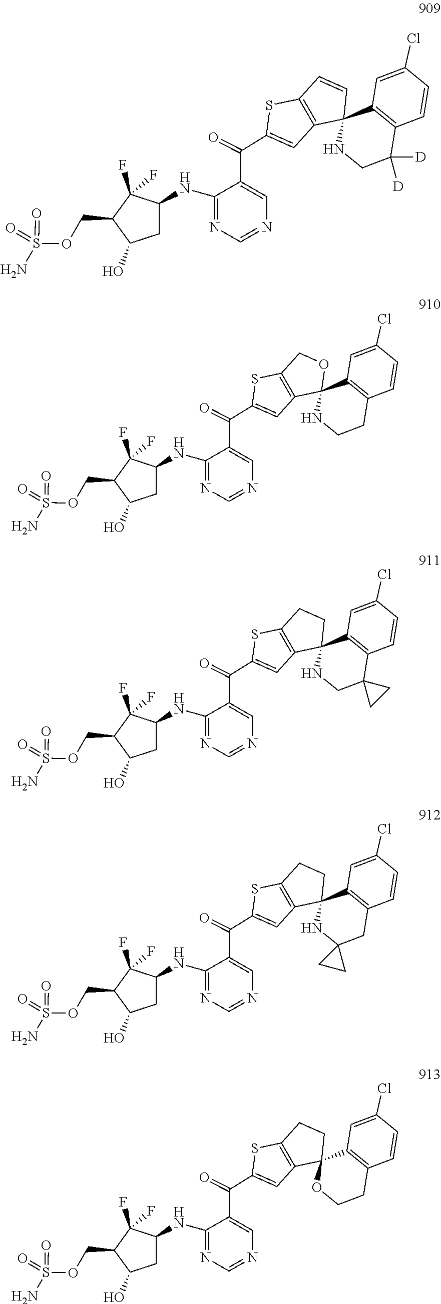

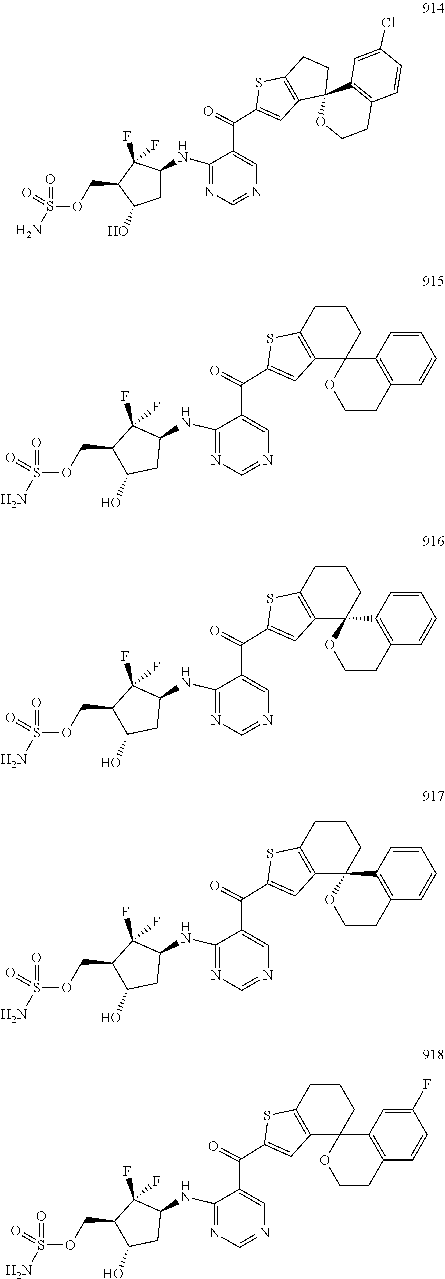

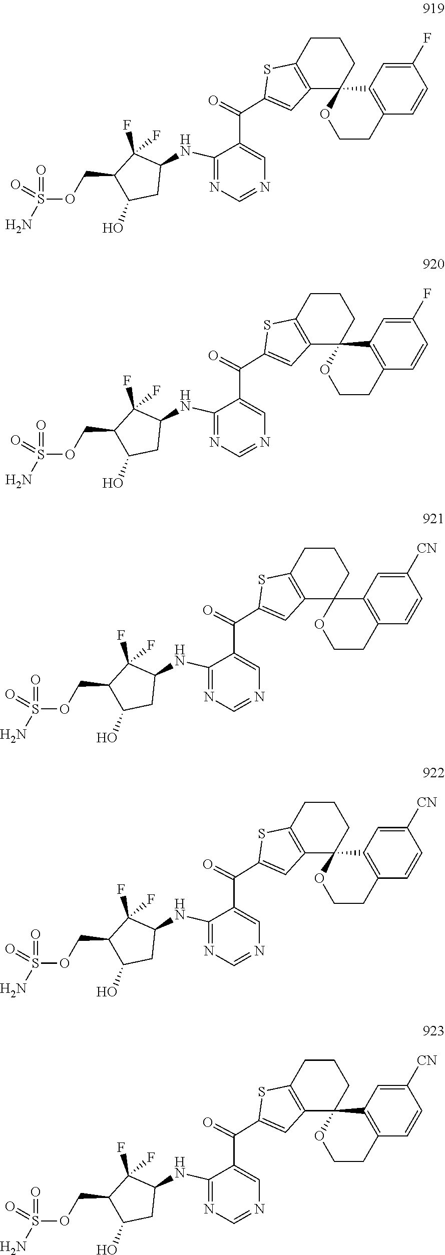

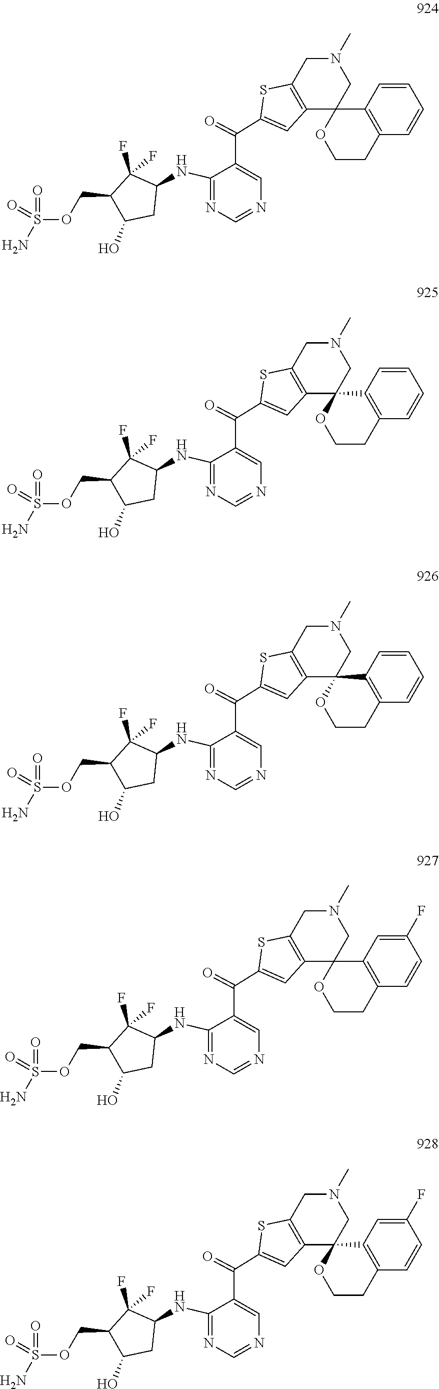

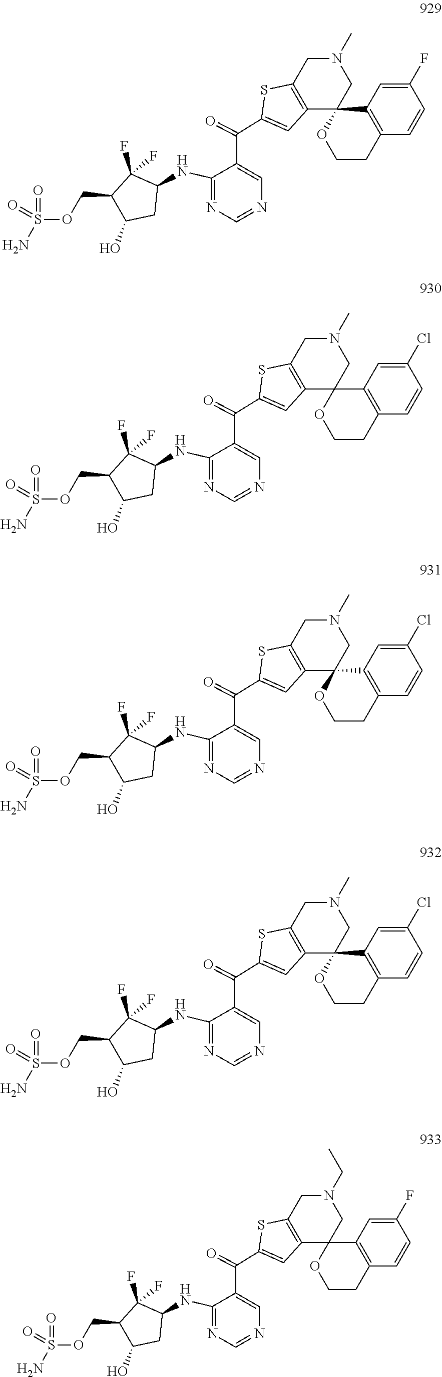

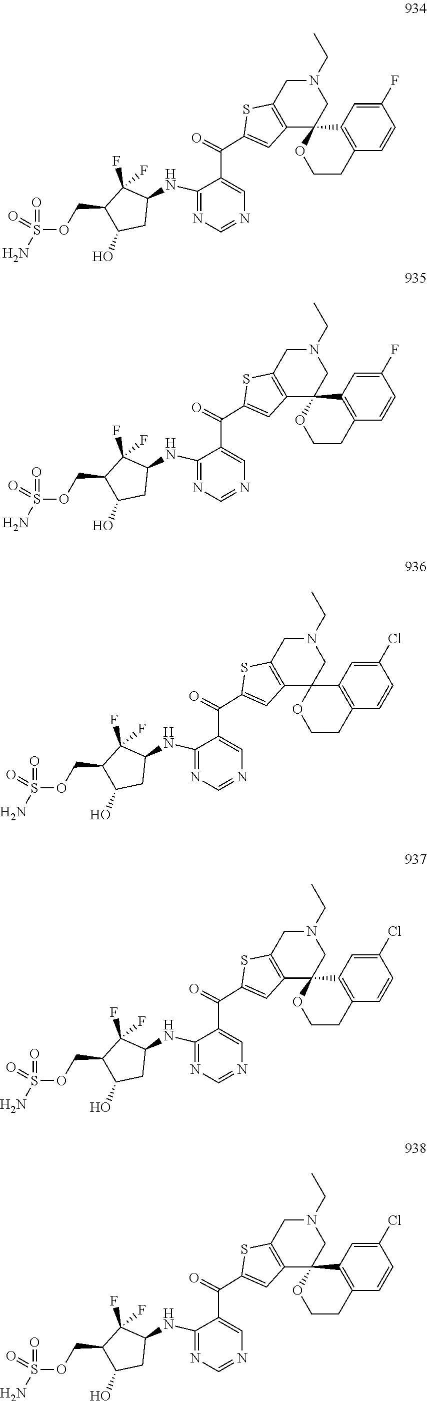

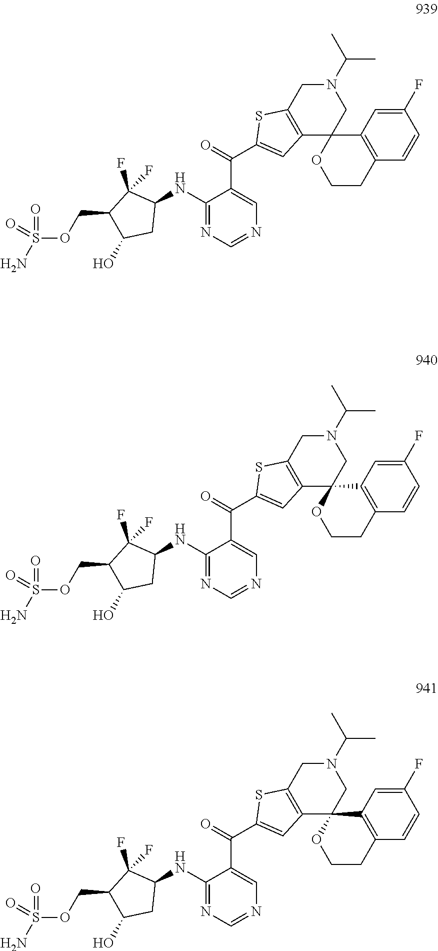

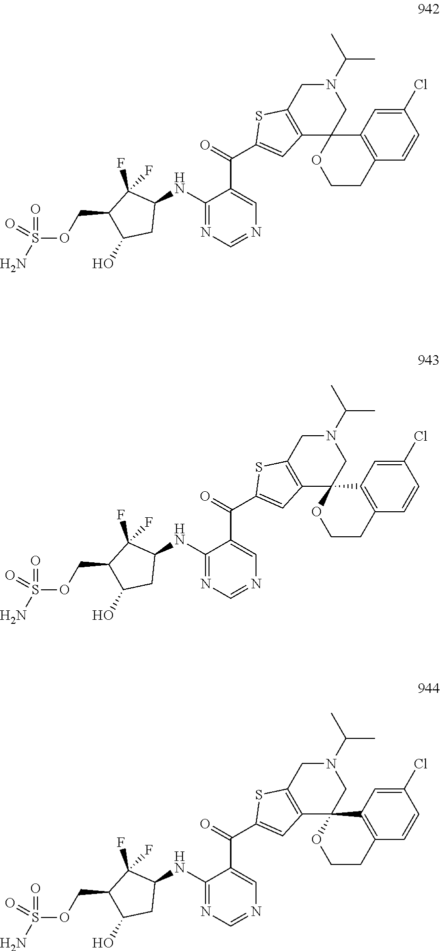

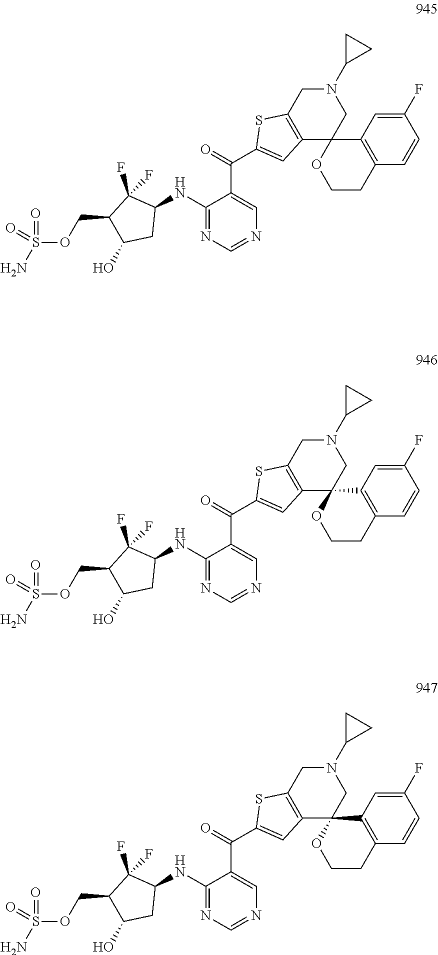

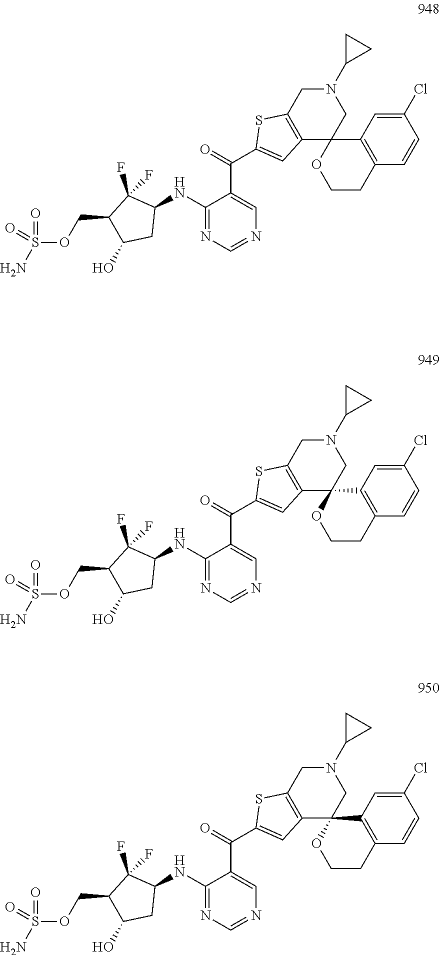

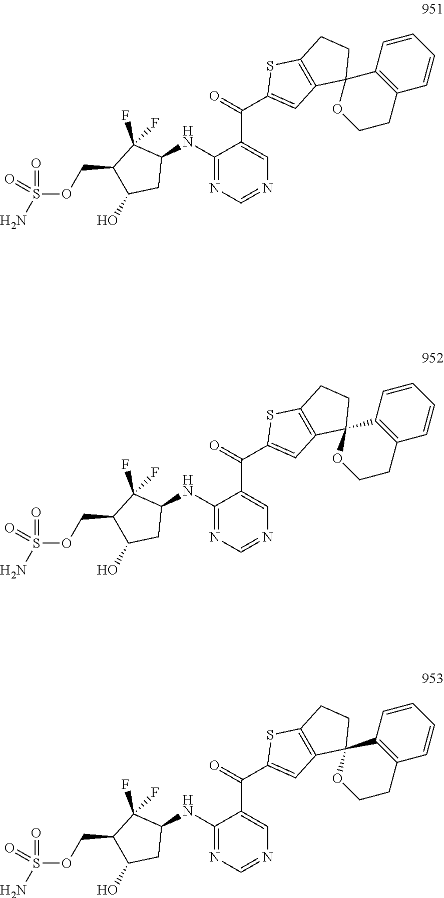

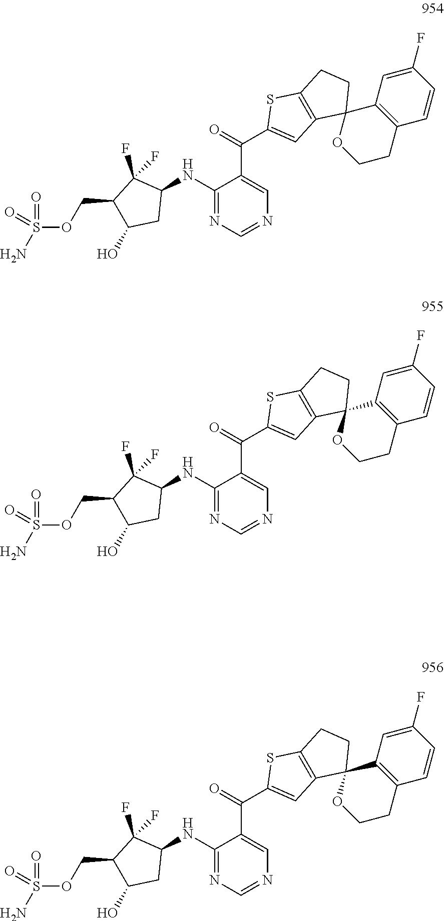

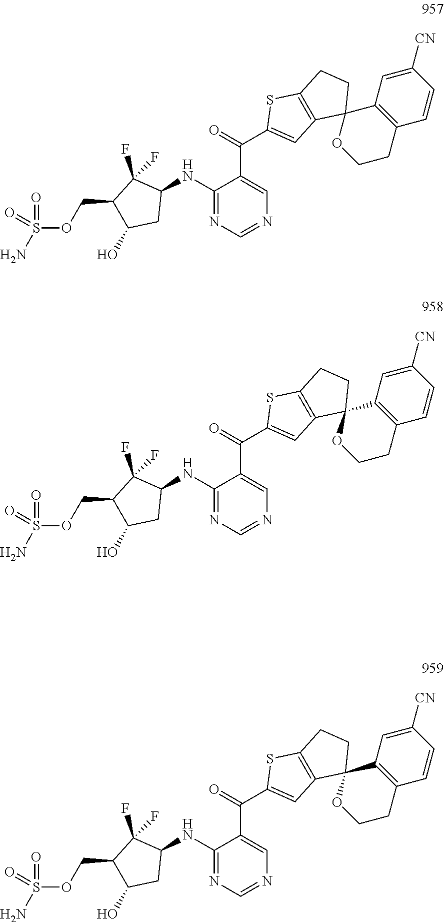

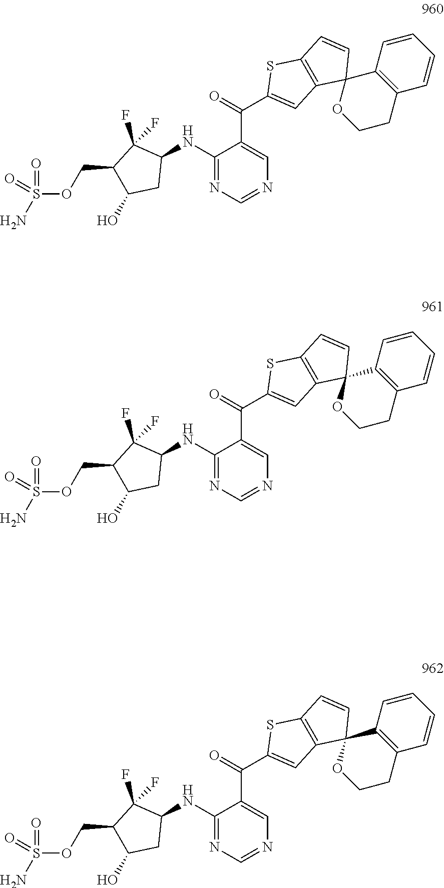

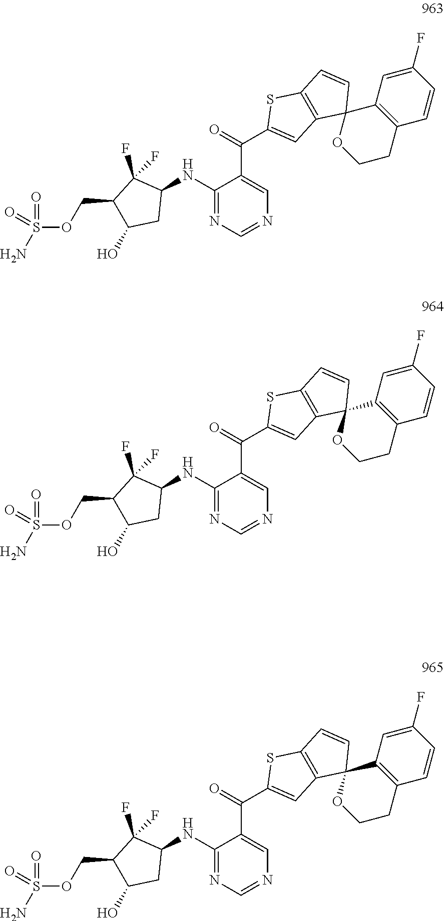

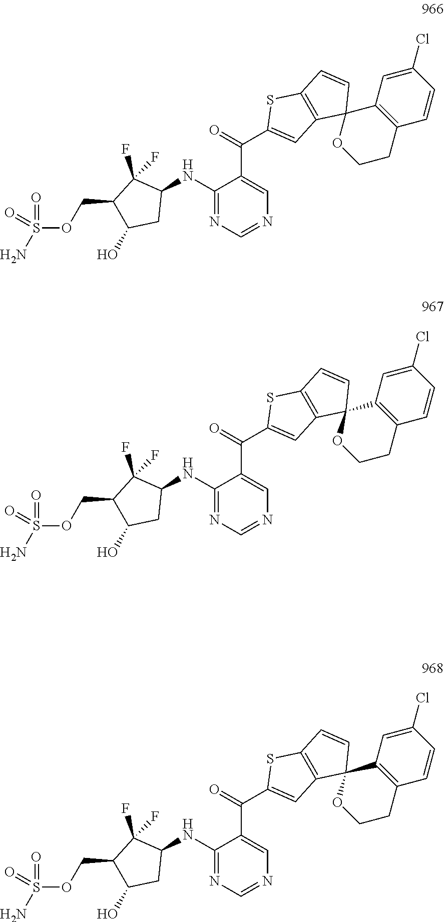

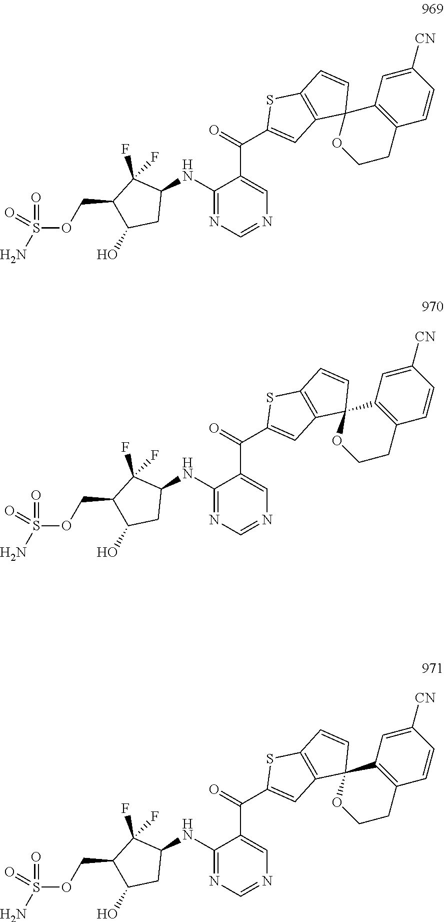

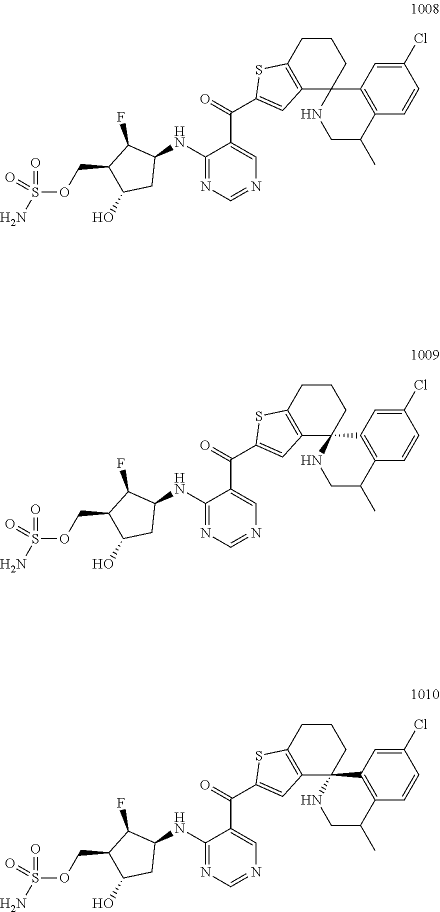

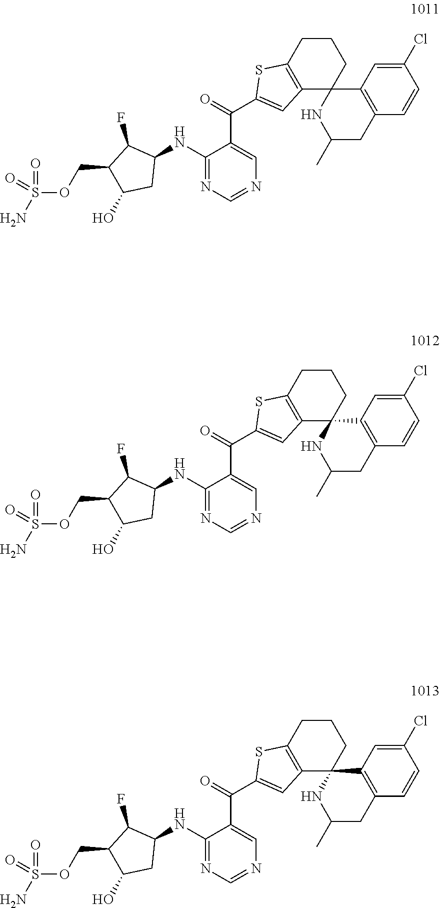

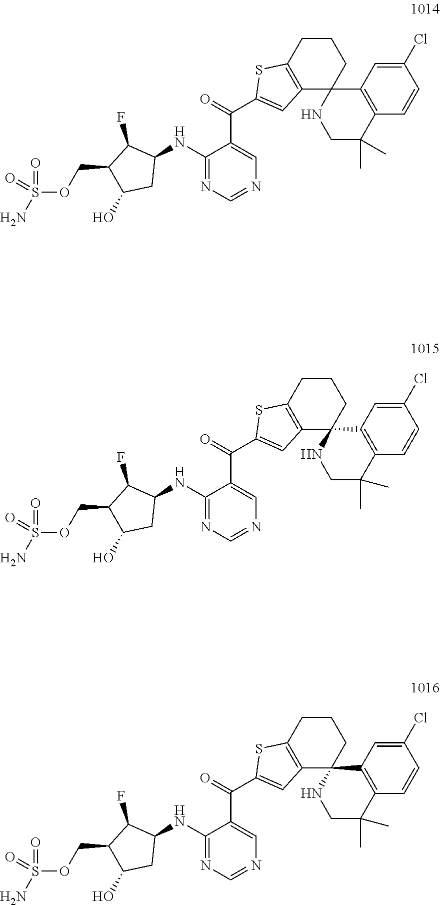

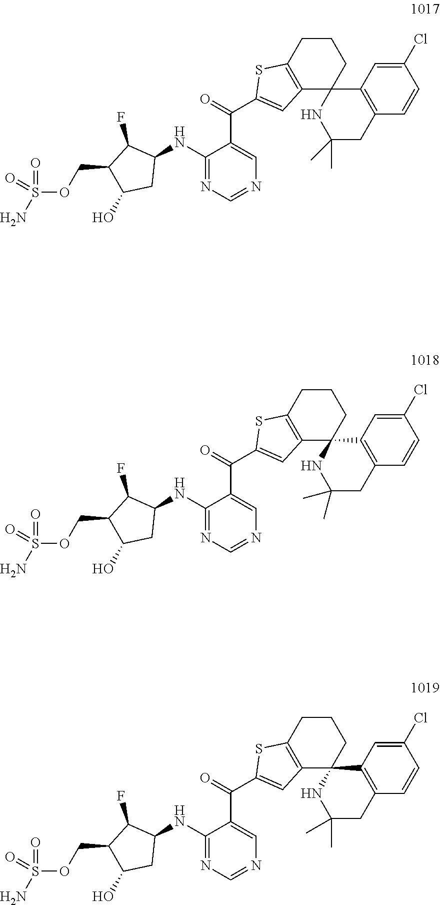

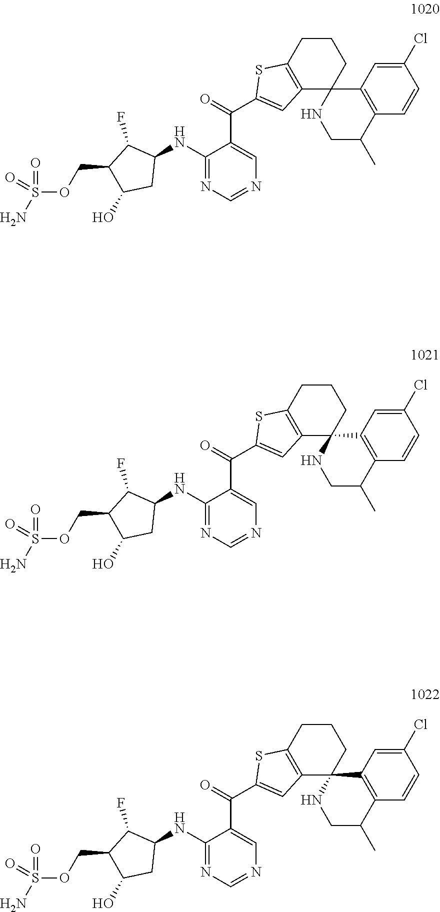

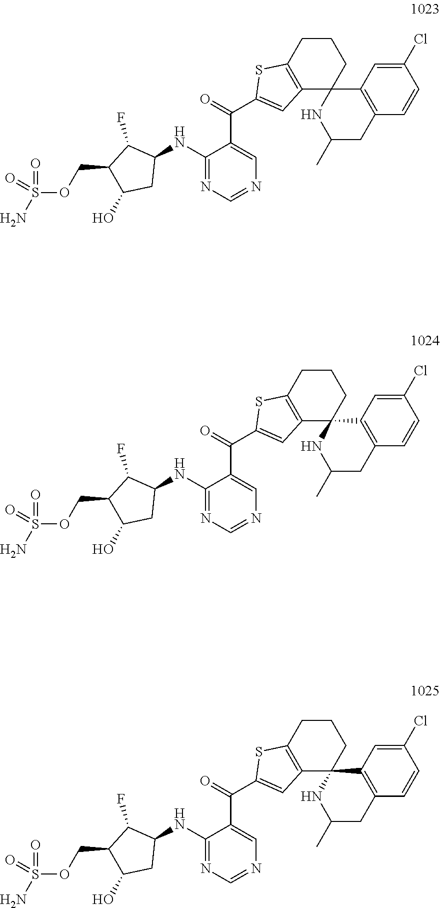

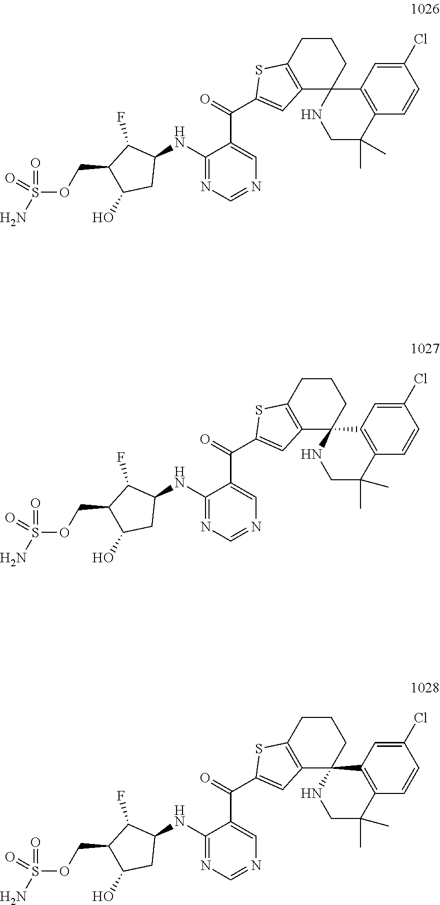

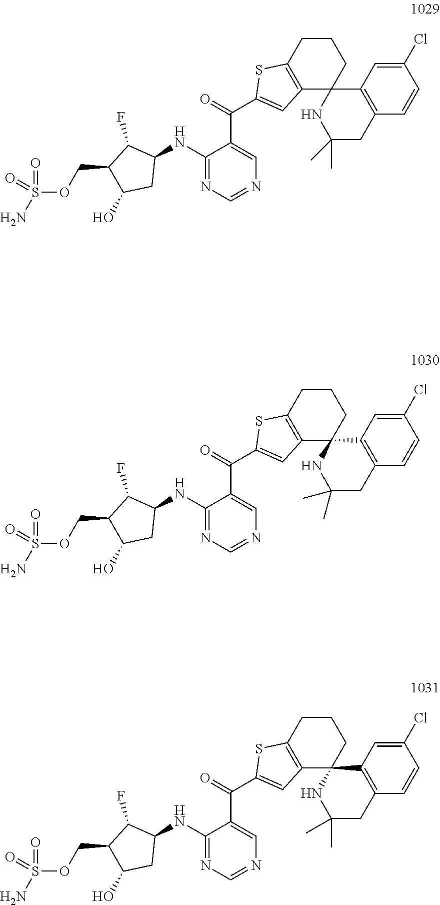

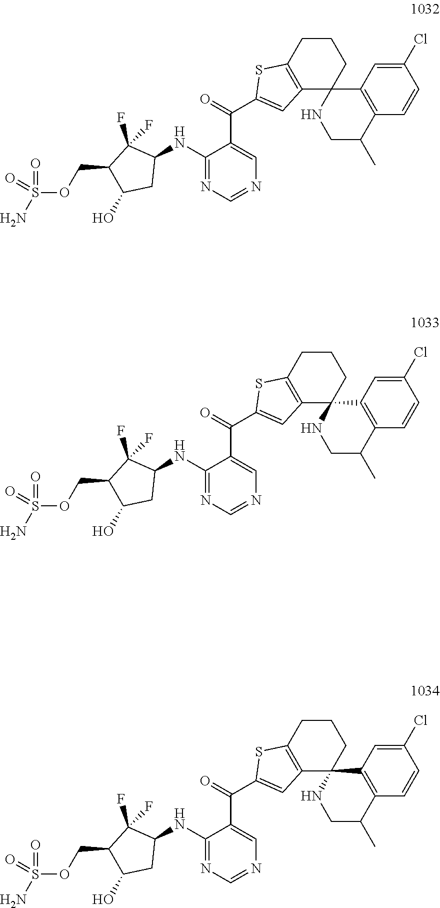

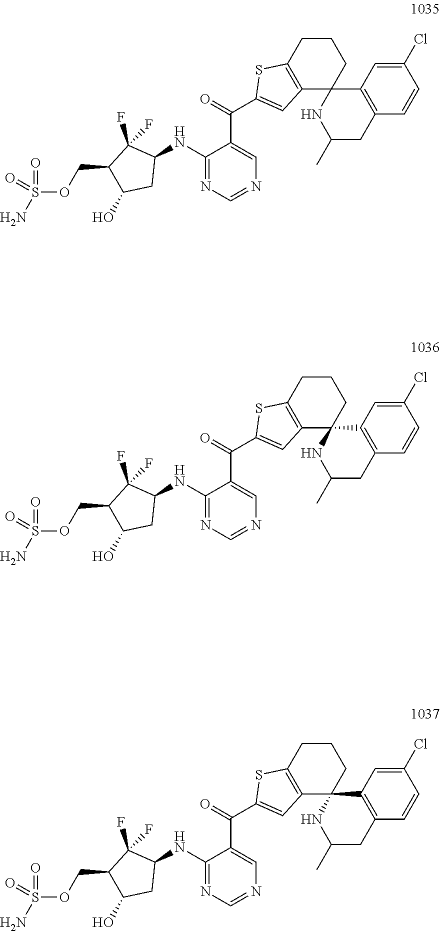

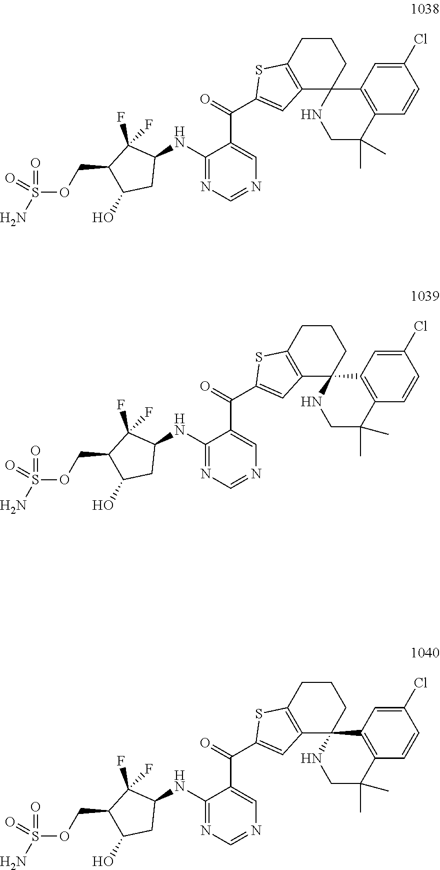

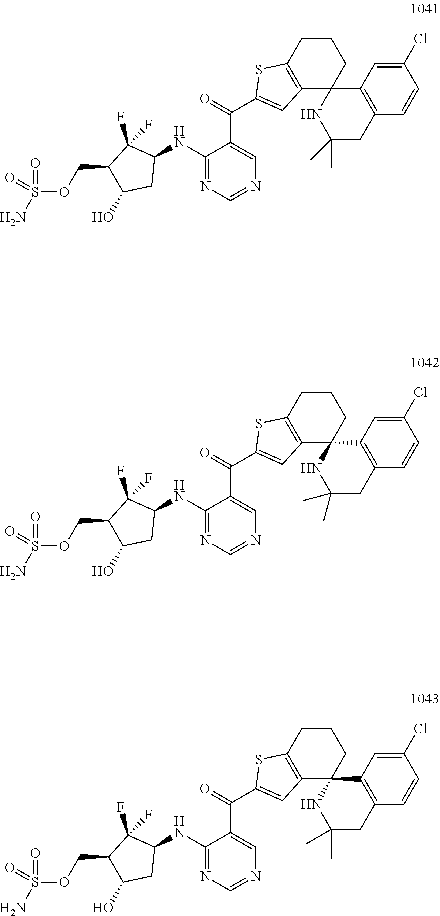

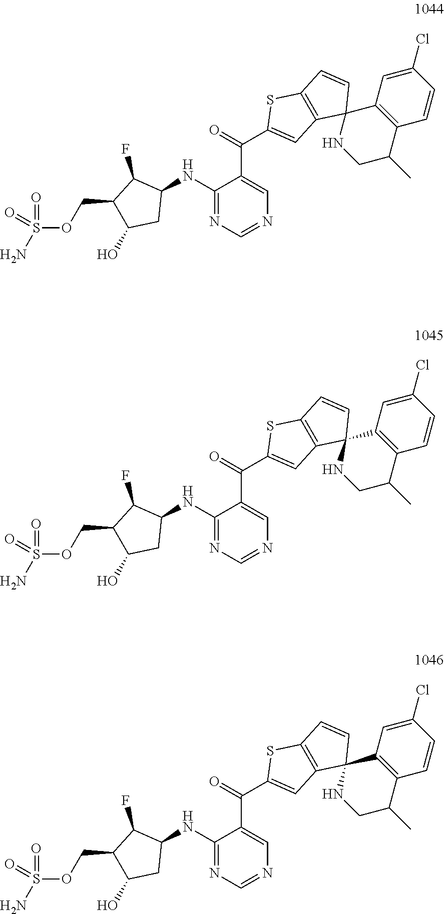

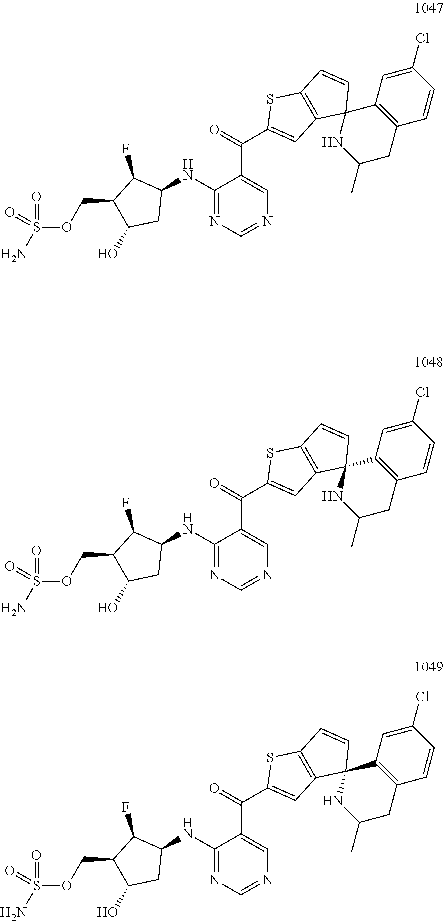

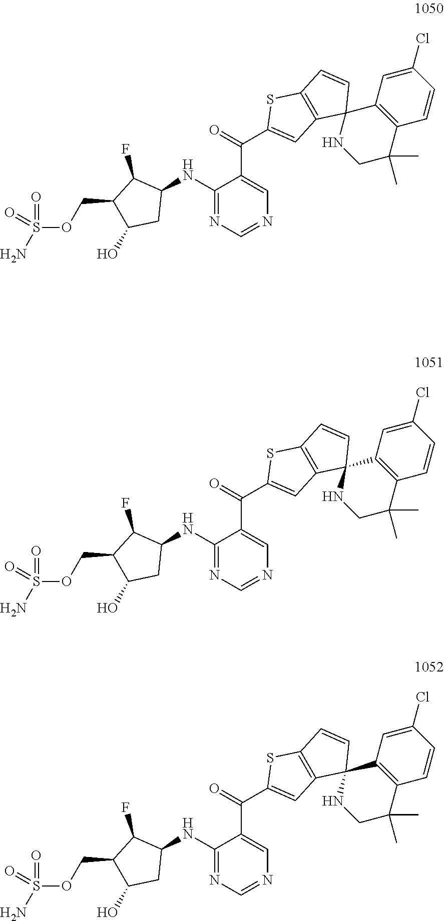

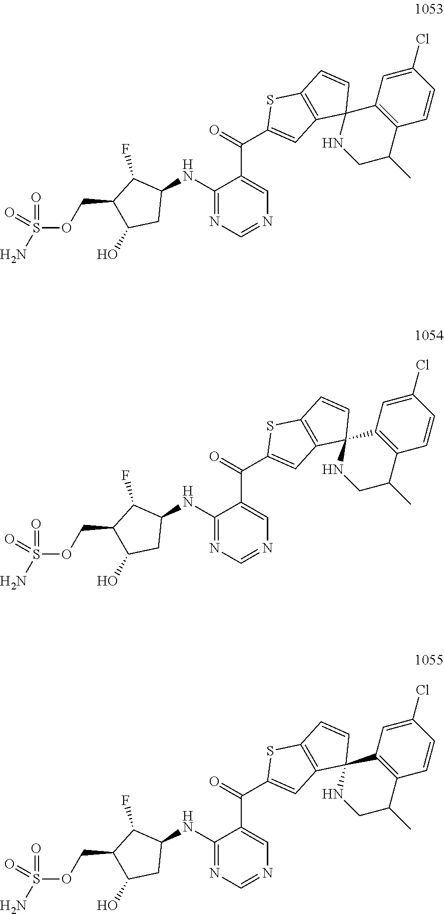

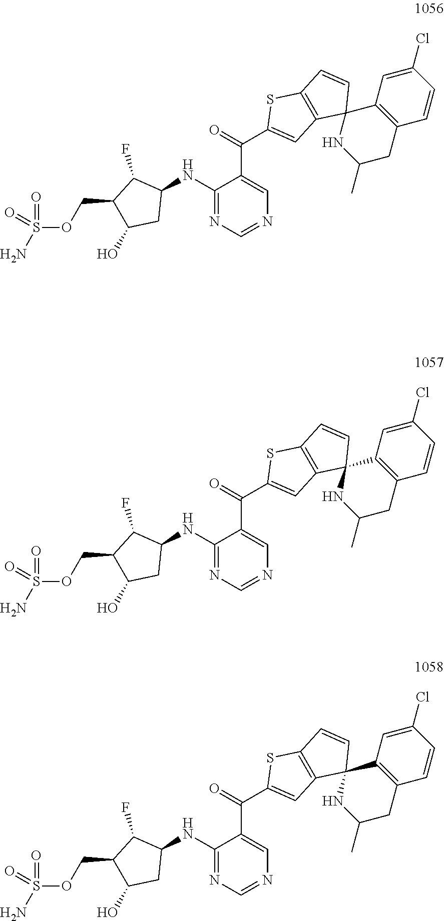

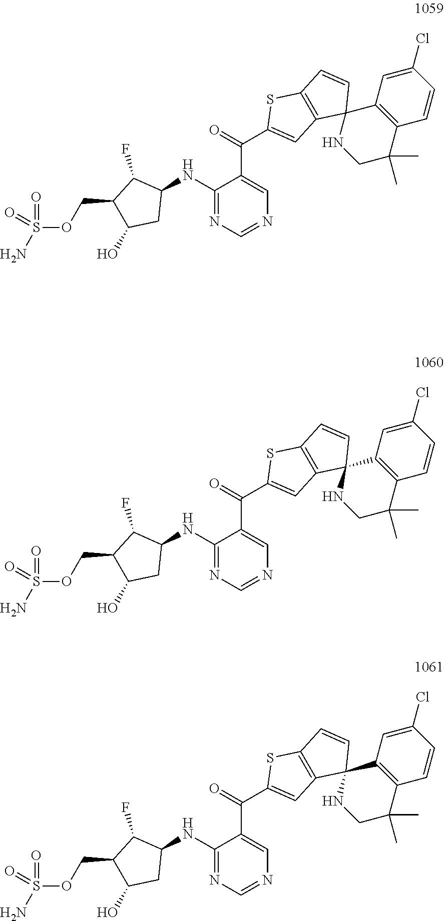

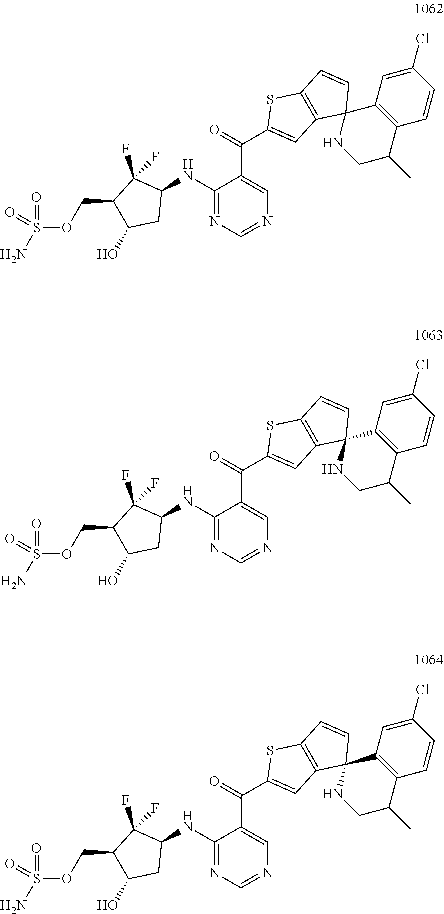

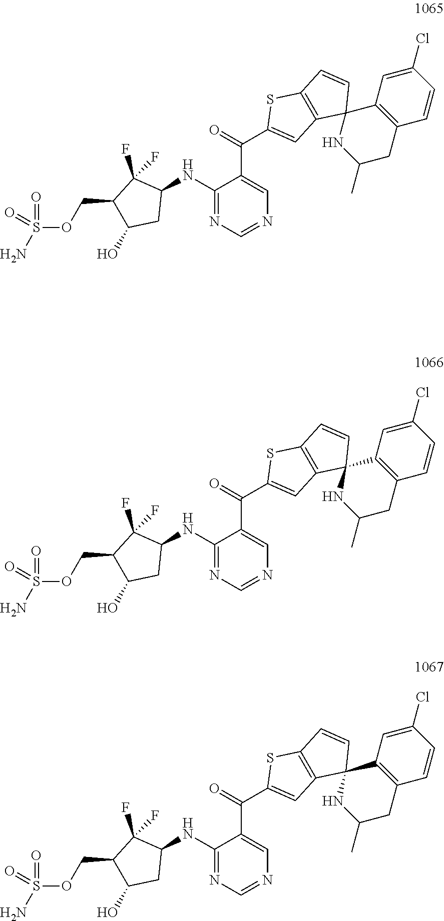

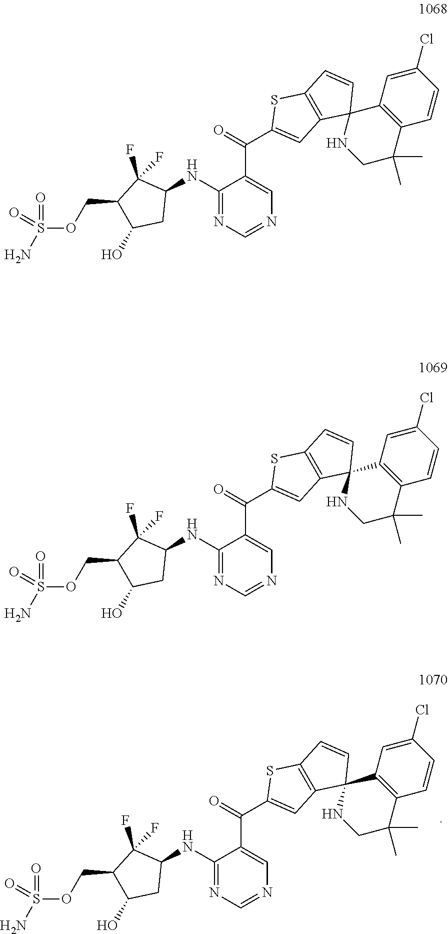

The present disclosure further provides a compound of general formula (2) or an isomer thereof, a crystalline form thereof, a pharmaceutically acceptable salt thereof, a hydrate thereof, or a solvate thereof:

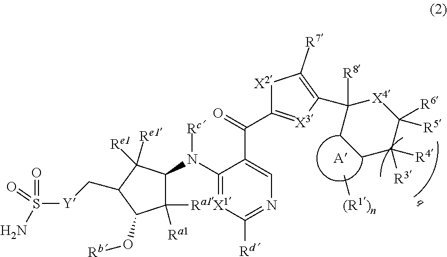

wherein in general formula (2):
Y is —O—, —CH₂—, or —N(H)—;
R^{a1} is —H, —F, —NH₂, or —OH;
R^{a1′} is —H or —F, and when R^{a1} is —NH₂ or —OH, R^{a1′} is —H;
R^{b′} is —H or (C1-C4) alkyl;
R^{e′} is —H or (C1-C4) alkyl;
R^{d′} is —H, halogen, —CF₃, or (C1-C4) alkyl;
R^{e1} and R^{e1′} are each independently and optionally —H or halogen, and R^{e1} and R^{e1′} are not both —H;
X^{1′} is C(H), C(F), or N;
X^{2′} is S or O;
X^{3′} is C(R^{x3′}) or N;
R^{x3′} is —H, halogen, or —CH₃;
X^{4′} is S, O, C(R^{x41a})(R^{x41b}), or N(R^{x42′});
R^{x42′} is —H, (C1-C4) alkyl, or (C3-C5) cycloalkyl;
R^{x41a} and R^{x41b} are each independently and optionally —H, halogen, —OH, —OR^{x411′}, —N(R^{x411′})(R^{x412′}), —CN, (C1-C6) alkyl, (C1-C6) haloalkyl, (C3-C9) cycloalkyl, or (C1-C6) alkoxy;
R^{x411′} and R^{x412′} are each independently and optionally —H, (C1-C4) alkyl, or (C3-C5) cycloalkyl, or R^{x41′} and
R^{x412′} on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H or halogen;
R^{3′} and R^{4′} are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR^{31′}, —(CH₂)ᵣNR^{31′}R^{32′}, OR^{31′}, NR^{31′}R^{32′}, —CN, —C(O)NR^{31′}R^{32′}, —NR^{32′}C(O)R^{31′}, —NR³²'S(O)₂R^{31′}, —S(O)ₚR^{31′}, and —S(O)₂NR^{31′}R^{32′}; orR^{3′} and R⁴, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or R^{3′} and the adjacent R^{5′}, together with the atom to which they are attached, can form a (C3-C9) cycloalkyl or (3- to 11-membered) heterocycloalkyl, wherein the (C3-C9) cycloalkyl or (3- to 11-membered) heterocycloalkyl may be each independently and optionally substituted with 1, 2, 3, or 4 of the following groups: —H, halogen, (C1-C6) alkyl, or (C1-C6) alkoxy; or when R^{3′} and the adjacent R^{5′} are both absent, an endocyclic double bond may be formed between the carbon atoms separately linked to R^{4′} and R^{6′}; or R^{3′} and R^{4′} together form an oxo; R^{5′} and R^{6′} are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR^{31′}, —(CH₂)ᵣNR^{31′}R^{32′}—OR^{31′}—NR^{31′}R^{32′}, —CN, —C(O)NR^{31′}R^{32′}, —NR^{32′}C(O)R^{31′}, —NR³²'S(O)₂R^{31′}, —S(O)ₚR^{31′}, and —S(O)₂NR^{31′}R^{32′}; or R¹ and R^{6′}, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, and (C1-C6) alkoxy; or R^{5′} and R^{6′} together form an oxo;
ring A′ is (C6-C10) aryl or (5- to 10-membered) heteroaryl;
each R^{1′} is independently and optionally: —H, halogen, —OH, —NO₂, —NR^{31′}R^{32′}, —(CH₂)ᵣOR^{31′}, —(CH₂)ᵣNR^{31′}R^{32′} —CN, (C1-C6) alkyl, (C1-C6) haloalkyl, (C1-C6) alkoxy, (C2-C6) alkenyl, (C2-C6) alkynyl, (C3-C8) cycloalkyl, —C(O)NR^{31′}R^{32′}, —NR^{32′}C(O)R^{31′}, —NR^{32′}S(O)₂R^{31′}, —S(O)ₚR^{31′}, or —S(O)₂NR^{31′}R^{32′}, wherein the (C1-C6) alkyl, (C1-C6) haloalkyl, (C1-C6) alkoxy, (C2-C6) alkenyl, (C2-C6) alkynyl, or (C3-C8) cycloalkyl may be each independently and optionally substituted with 1, 2, 3, or 4 of the following groups: —H, halogen, —OH, —(CH₂)ᵣOR^{31′}, —(CH₂)ᵣNR^{31′}R^{32′}, —OR^{31′}, —NR^{31′}R^{32′}, —CN, and (C1-C6) alkyl;
R^{7′} is: —H, -D, halogen, —OH, —NR^{31′}R^{32′}, —CN, (C1-C6) alkyl, (C1-C6) haloalkyl, (C1-C6) alkoxy, (C2-C6) alkenyl, (C2-C6) alkynyl, or (C3-C8) cycloalkyl;
R^{8′} is: —H, -D, —OH, (C1-C6) alkyl, (C2-C6) alkenyl, (C2-C6) alkynyl, or (C3-C8) cycloalkyl;
R^{31′} and R^{32′} are each independently and optionally —H, (C1-C4) alkyl, or (C3-C5) cycloalkyl, or R^{31′} and R^{32′} on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H and halogen; and
n is an integer of 0, 1, 2, 3, or 4, r is an integer of 0, 1, or 2, p is an integer of 0, 1, or 2, and q is an integer of 0, 1, or 2.

In another preferred embodiment, in general formula (2), R^{d′} is —H, —F, —CF₃, or —CH₃.

In another preferred embodiment, in general formula (2), R^{e1′} and R^{e1′} are each independently and optionally —H or —F, and R^{e1} and R^{e1′} are not both —H.

In another preferred embodiment, in general formula (2), R^{x42′} is —H, (C1-C3) alkyl, or (C3-C5) cycloalkyl.

In another preferred embodiment, in general formula (2), R^{x42′} is —H,

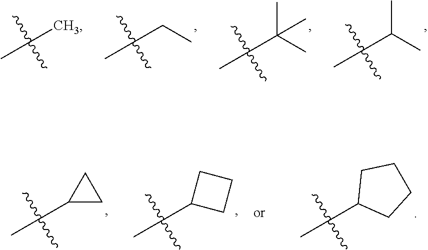

In another preferred embodiment, in general formula (2), R^{41a} and R^{x41b} are each independently and optionally —H, —F, —OH, —OCH₃, —N(CH₃)₂, —CN,

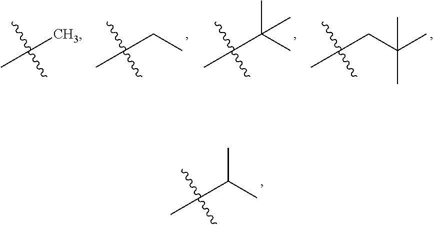

—CF₃, —CH₂CF₃,

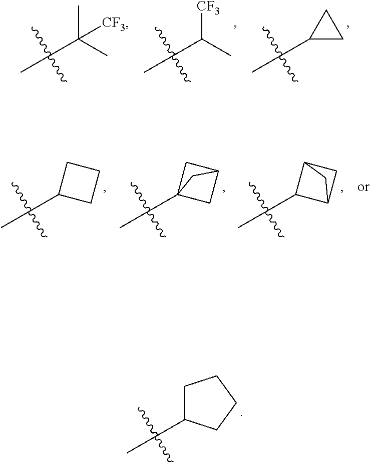

In another preferred embodiment, in general formula (2), R^{3′} and R^{4′} are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R^{3′} and R^{4′}, together with the carbon atom to which they are attached, can form (4- to 6-membered) heterocycloalkyl or (C3-C4) cycloalkyl, wherein the (4- to 6-membered) heterocycloalkyl or (C3-C4) cycloalkyl may be optionally substituted with 1, 2, 3, or 4 of the following groups: —H, —F,

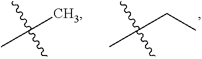

or —OCH₃; or R^{3′} and the adjacent R^{5′}, together with the atom to which they are attached, can form a (C3-C6) cycloalkyl or (3- to 6-membered) heterocycloalkyl, wherein the (C3-C6) cycloalkyl or (3- to 6-membered) heterocycloalkyl may be each independently and optionally substituted with 1, 2, 3, or 4 of the following groups: —H, —F,

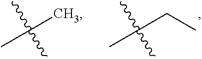

or —OCH₃; or when R^{3′} and the adjacent R^{5′} are both absent, an endocyclic double bond may be formed between the carbon atoms separately linked to R^{4′} and R^{6′}; or R^{3′} and R^{4′} together form an oxo.

In another preferred embodiment, in general formula (2), R^{5′} and R^{6′} are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R⁵ and R⁶, together with the carbon atom to which they are attached, can form a (4- to 6-membered) heterocycloalkyl or (C3-C4) cycloalkyl, wherein the (4- to 6-membered) heterocycloalkyl or C3-C4) cycloalkyl may be optionally substituted with 1, 2, 3, or 4 of the following groups: —H, —F,

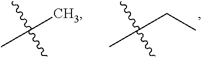

or —OCH₃; or R^{5′} and R^{6′} together form an oxo.

In another preferred embodiment, in general formula (2), ring A′ is phenyl or (5- to 6-membered) heteroaryl.

In another preferred embodiment, in general formula (2), ring A′ is

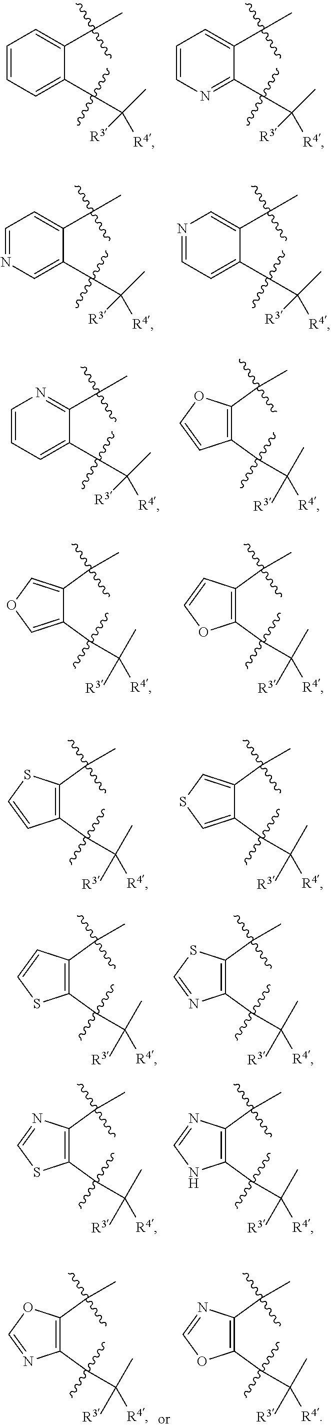

In another preferred embodiment, in general formula (2), each R^{1′} is independently and optionally: —H, —F, —Cl, —Br, —I, —OH, —NO₂, —N(CH₃)₂, —NH₂, —CH₂OCH₃, —CH₂N(CH₃)₂, —CN, (C1-C3) alkyl, (C1-C3) haloalkyl, (C1-C3) alkoxy, (C2-C4) alkenyl, (C2-C4) alkynyl, (C3-C6) cycloalkyl, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂, wherein the (C1-C3) alkyl, (C1-C3) haloalkyl, (C1-C3) alkoxy, (C2-C4) alkenyl, (C2-C4) alkynyl, or (C3-C6) cycloalkyl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN,

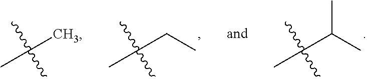

In another preferred embodiment, in general formula (2), each R^{1′} is independently: —H, —F, —Cl, —Br, —I, —OH, —NO₂, —N(CH₃)₂, —NH₂, —CH₂OCH₃, —CH₂N(CH₃)₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, —S(O)₂N(CH₃)₂,

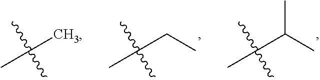

—CF₃, —CH₂CF₃,

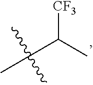 [SMILES: *C(C)C(F)(F)F]

—OCH₃, —OCH₂CH₃, —OCH(CH₃)₂,

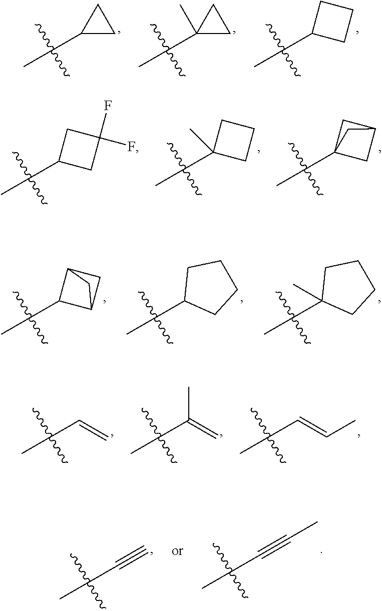

In another preferred embodiment, in general formula (2), R^{7′} is: —H, -D, —F, —Cl, —Br, —I, —OH, —N(CH₃)₂, —NH₂, —CN, (C1-C3) alkyl, (C1-C3) haloalkyl, (C1-C3) alkoxy, (C2-C4) alkenyl, (C2-C4) alkynyl, or (C3-C5) cycloalkyl.

In another preferred embodiment, in general formula (2), R^{7′} is: —H, -D, —F, —Cl, —Br, —I, —OH, —N(CH₃)₂, —NH₂, —CN,

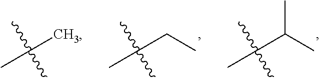

—CF₃, —CH₂CF₃,

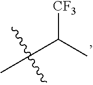 [SMILES: *C(C)C(F)(F)F]

—OCH₃, —OCH₂CH₃, —OCH(CH₃)₂,

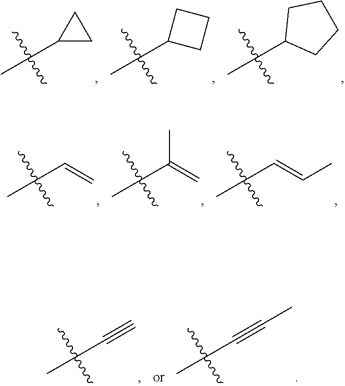

In another preferred embodiment, in general formula (2), R^{8′} is: —H, -D, —OH, (C1-C3) alkyl, (C2-C4) alkenyl, (C2-C4) alkynyl, or (C3-C5) cycloalkyl.

In another preferred embodiment, in general formula (2), R^{8′} is: —H, -D, C

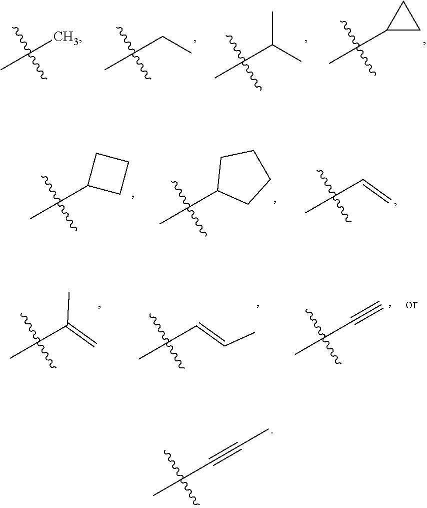

In another preferred embodiment, in general formula (2), the structural unit

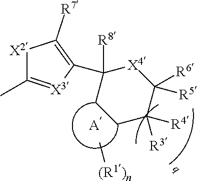

is

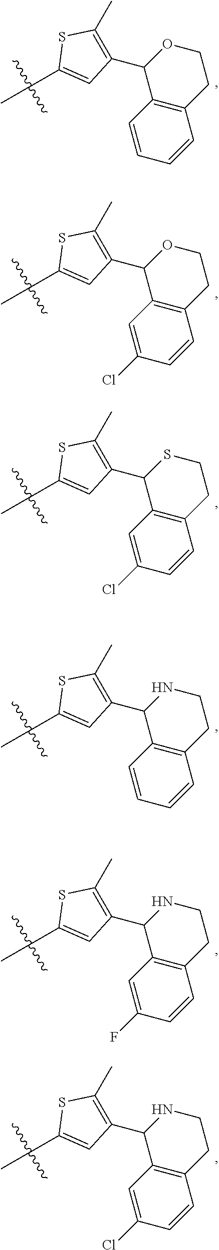

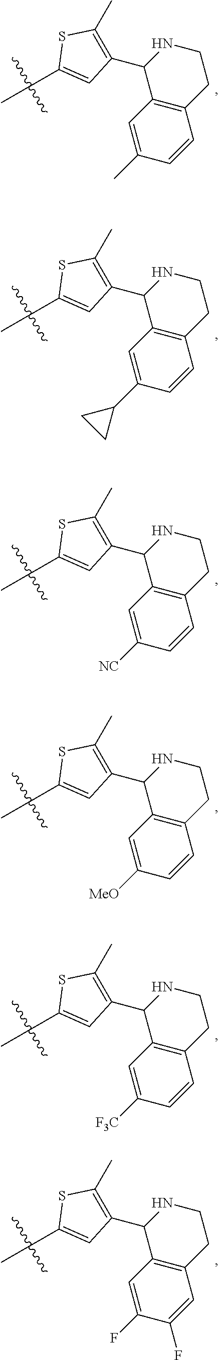

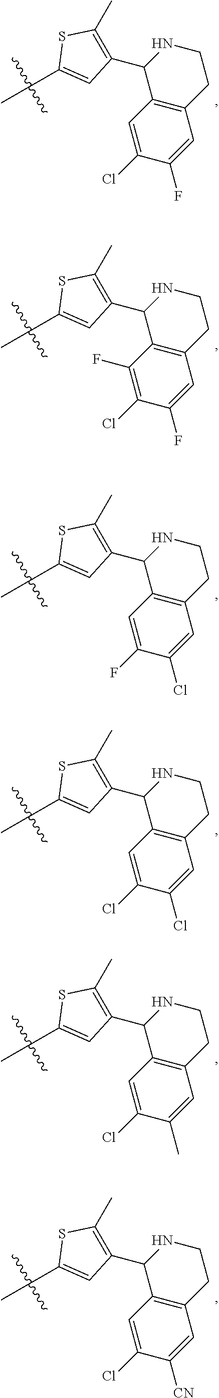 [SMILES: *c1cc(C2NCCc3cc(F)c(Cl)cc32)c(C)s1 ; *c1cc(C2NCCc3cc(F)c(Cl)c(F)c32)c(C)s1 ; *c1cc(C2NCCc3cc(Cl)c(F)cc32)c(C)s1 ; *c1cc(C2NCCc3cc(Cl)c(Cl)cc32)c(C)s1 ; *c1cc(C2NCCc3cc(C)c(Cl)cc32)c(C)s1 ; *c1cc(C2NCCc3cc(C#N)c(Cl)cc32)c(C)s1]

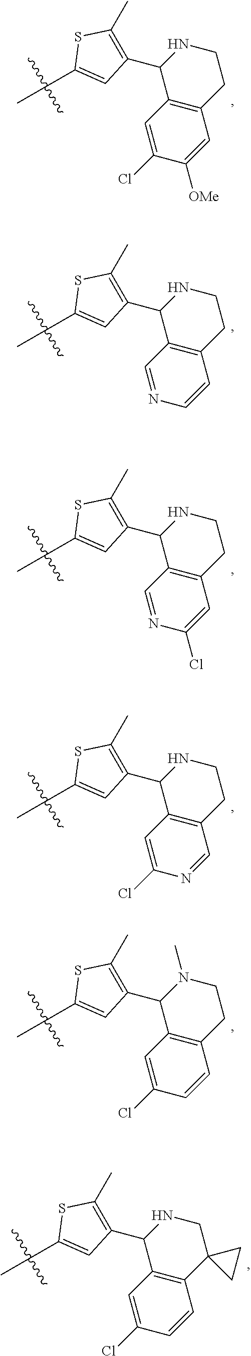

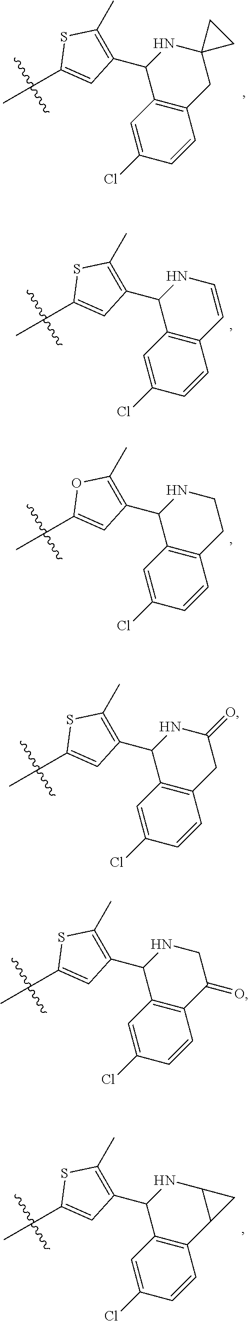

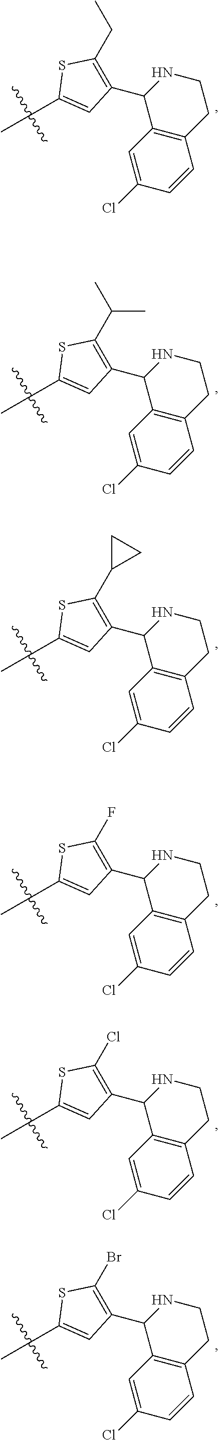

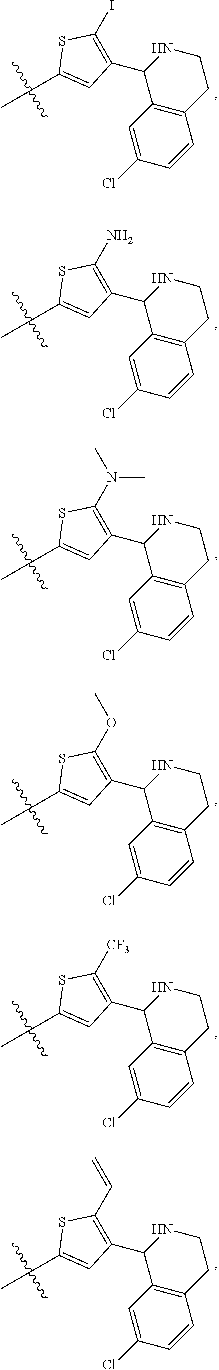

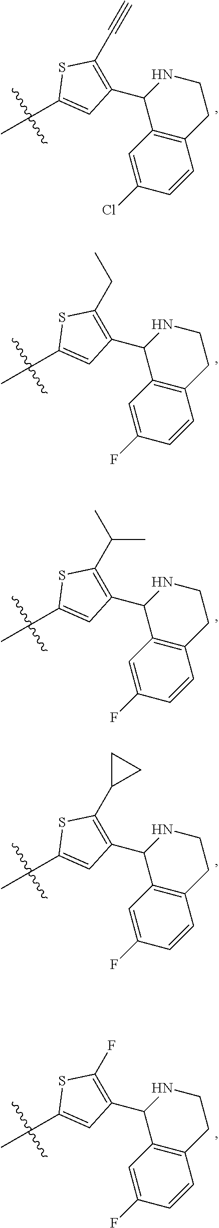

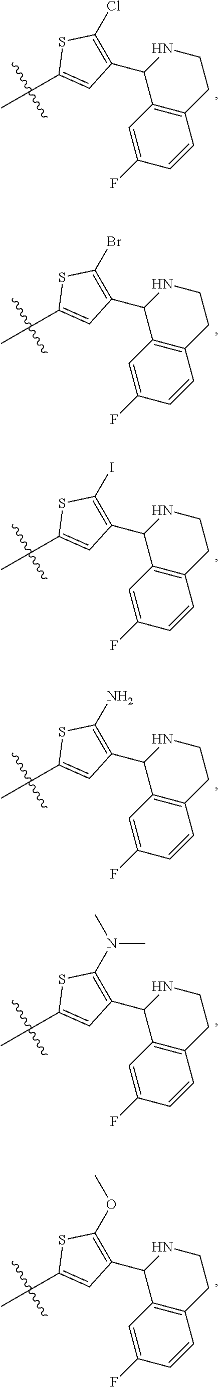

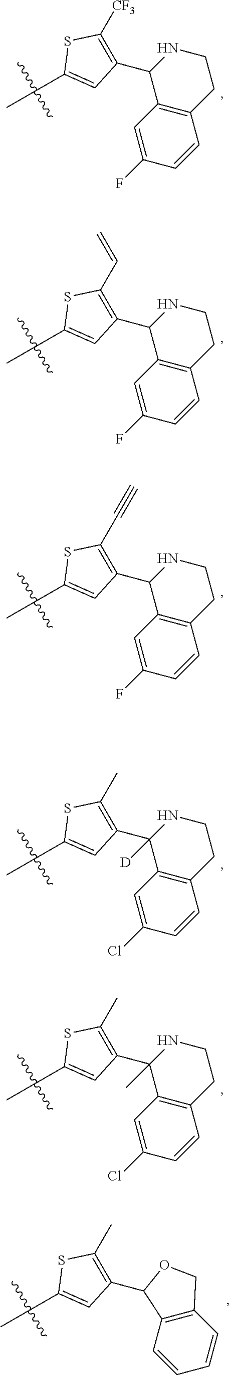 [SMILES: *c1cc(C2NCCc3ccc(F)cc32)c(C(F)(F)F)s1 ; *c1cc(C2NCCc3ccc(F)cc32)c(C=C)s1 ; *c1cc(C2NCCc3ccc(F)cc32)c(C#C)s1 ; *c1cc(C2([2H])NCCc3ccc(Cl)cc32)c(C)s1 ; *c1cc(C2(C)NCCc3ccc(Cl)cc32)c(C)s1 ; *c1cc(C2OCc3ccccc32)c(C)s1]

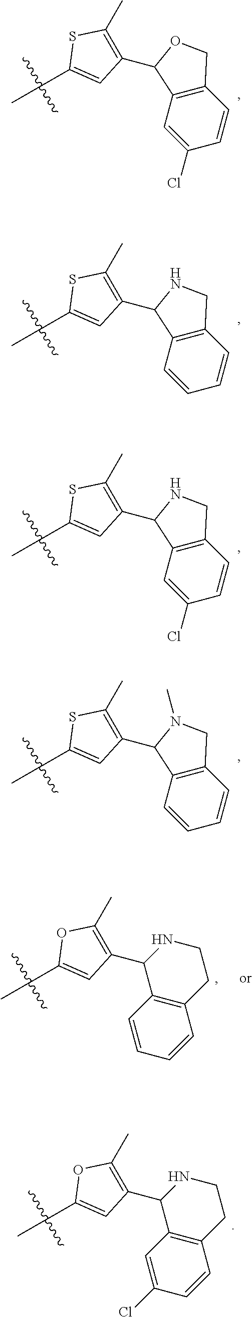 [SMILES: *c1cc(C2OCc3ccc(Cl)cc32)c(C)s1 ; *c1cc(C2NCc3ccccc32)c(C)s1 ; *c1cc(C2NCc3ccc(Cl)cc32)c(C)s1 ; *c1cc(C2c3ccccc3CN2C)c(C)s1 ; *c1cc(C2NCCc3ccccc32)c(C)o1 ; *c1cc(C2NCCc3ccc(Cl)cc32)c(C)o1]

In another specific embodiment of the present disclosure, the compound of general formula (2) has one of the following structures

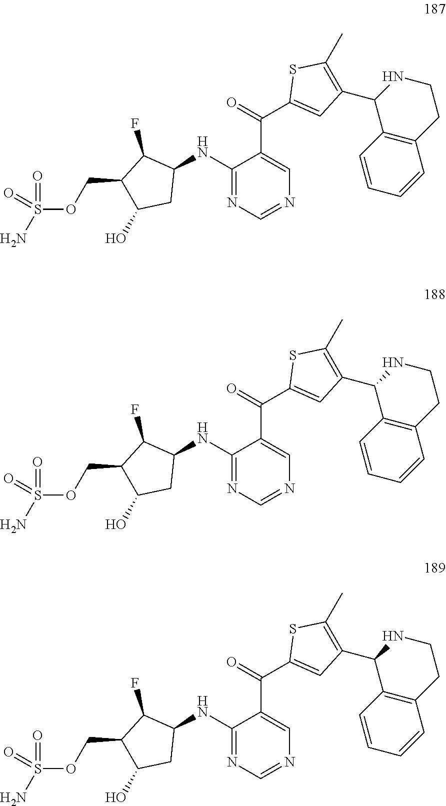

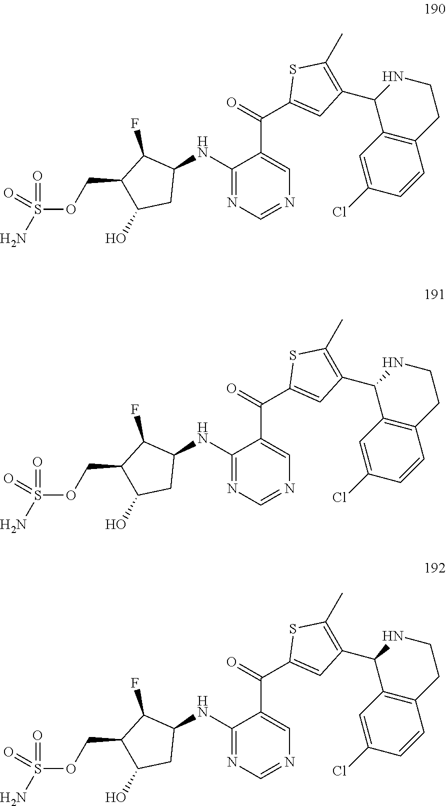

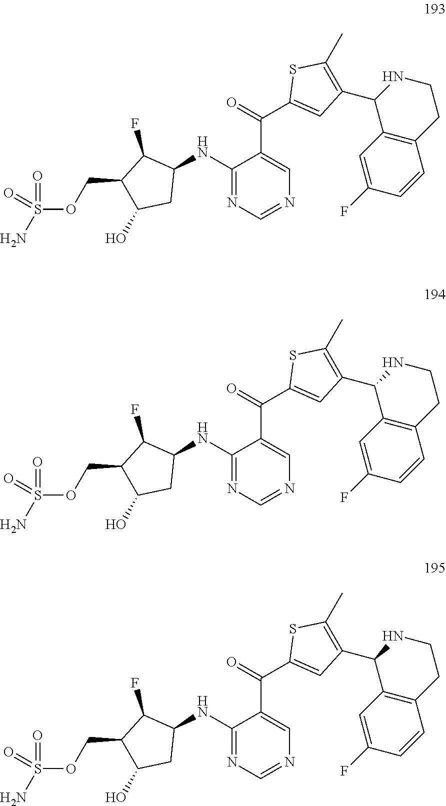

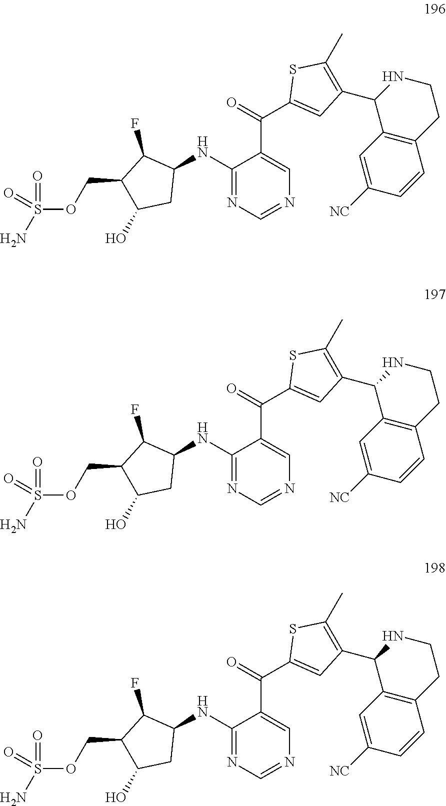

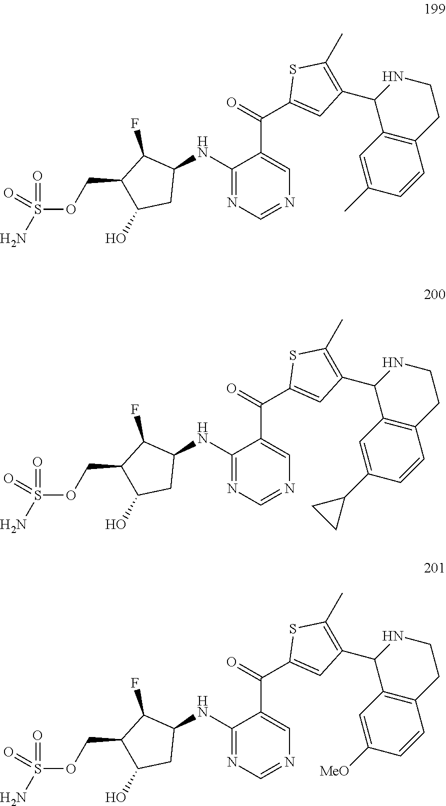

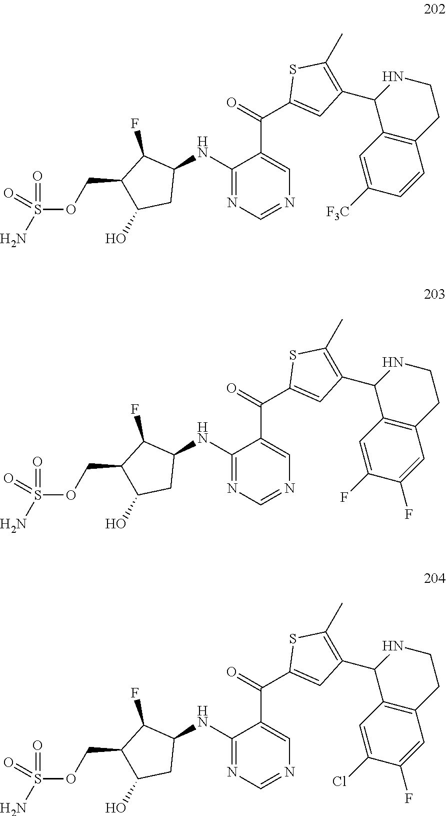

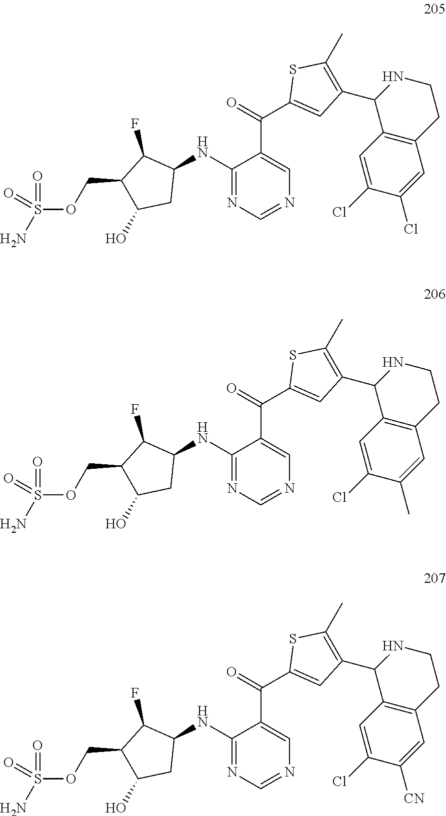

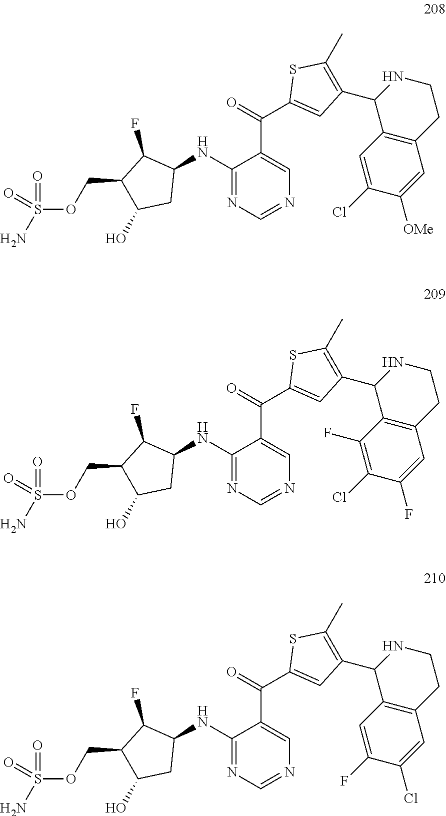

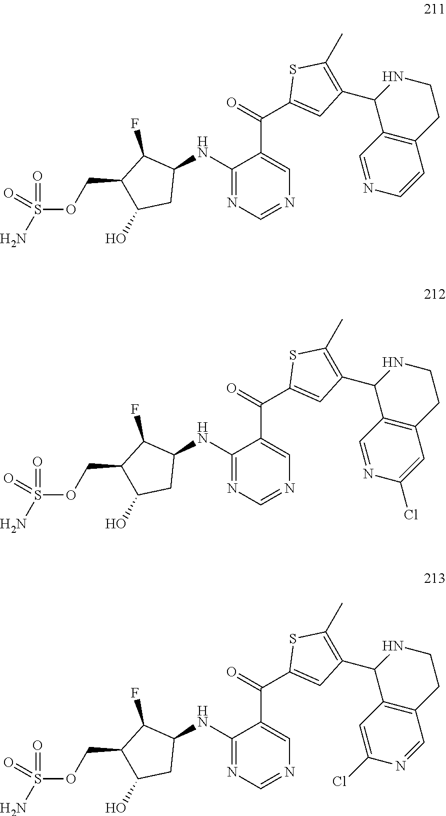

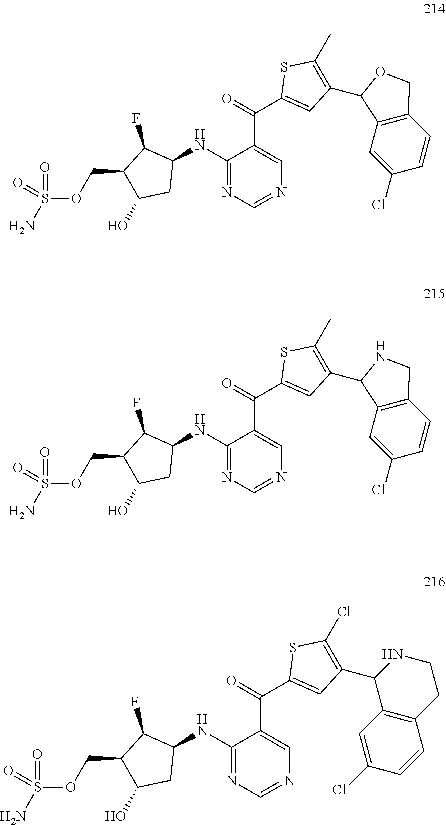

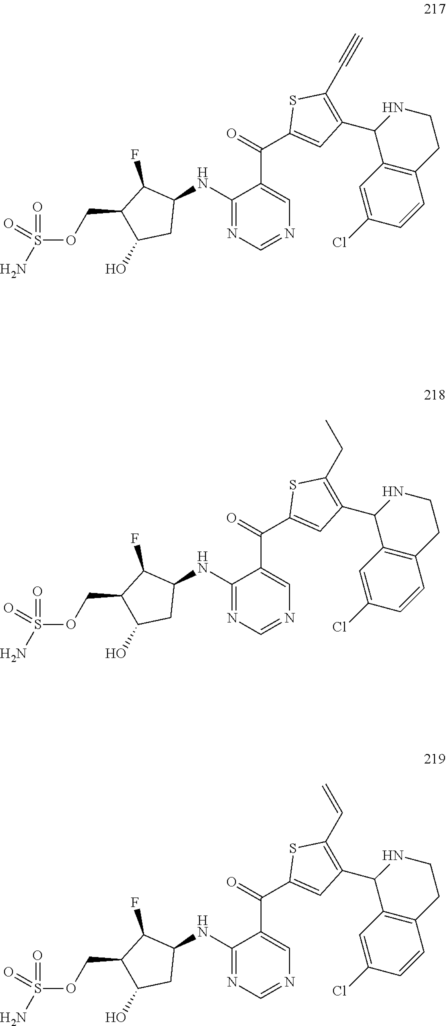

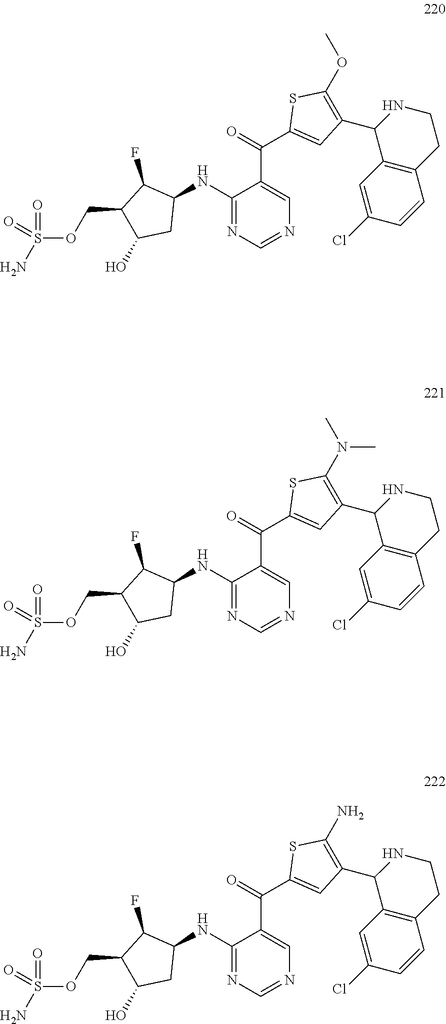

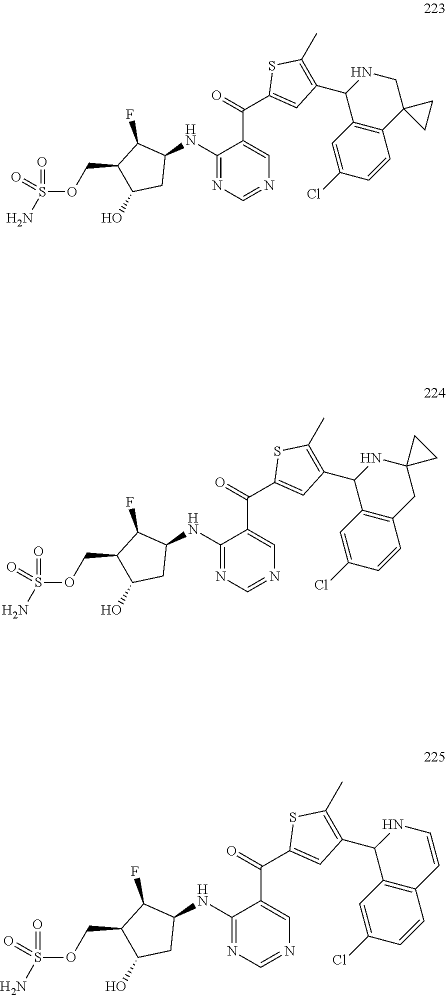

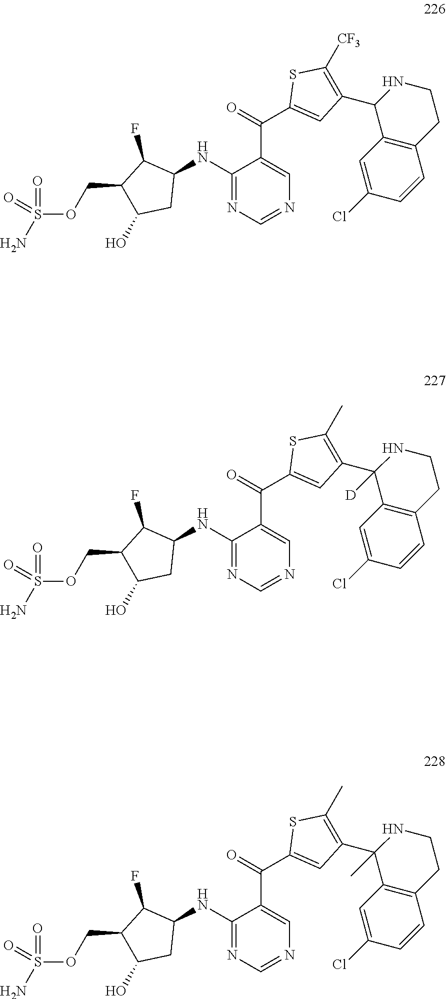

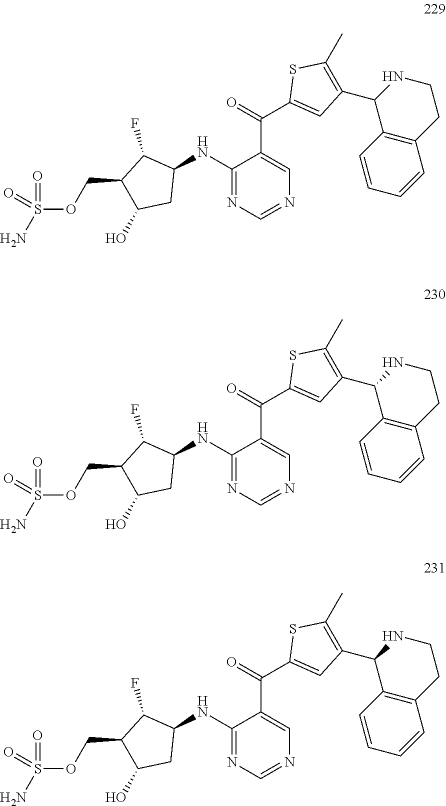

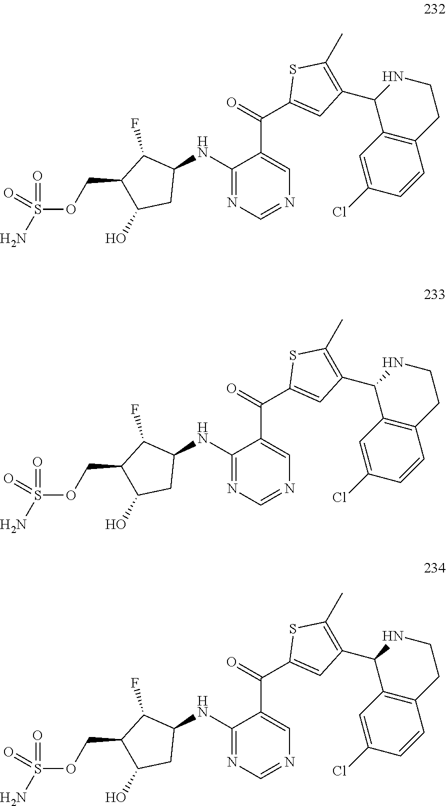

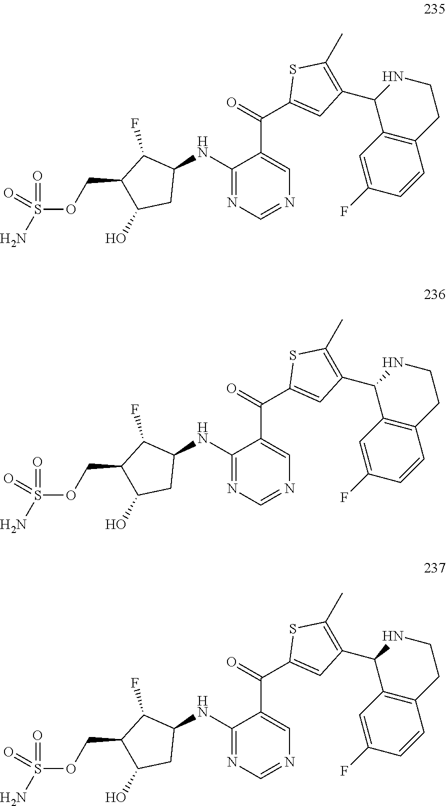

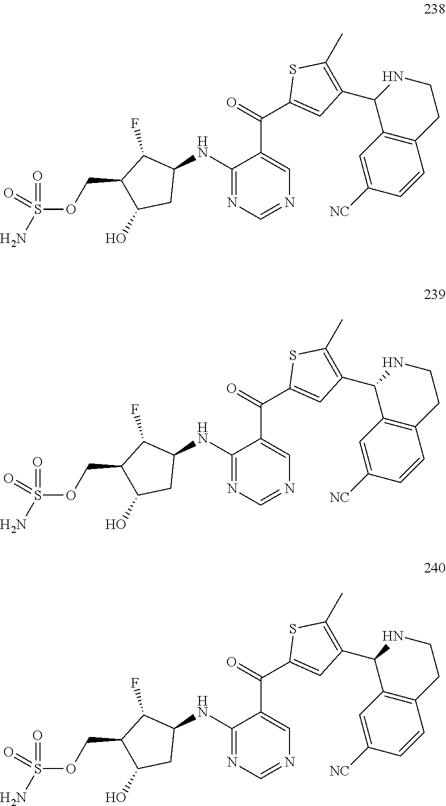

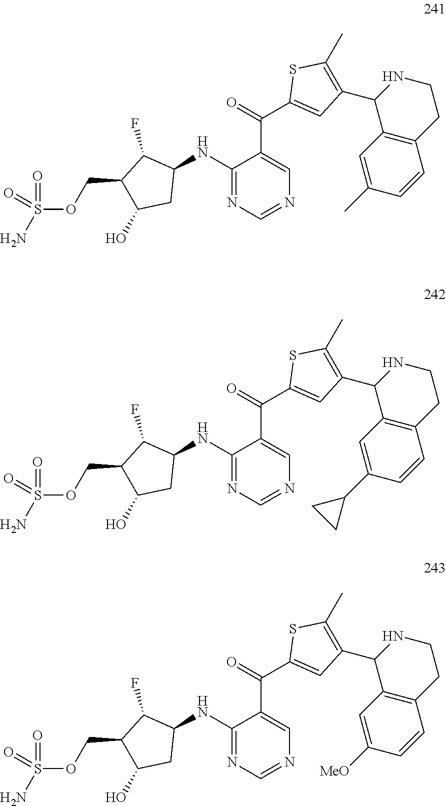

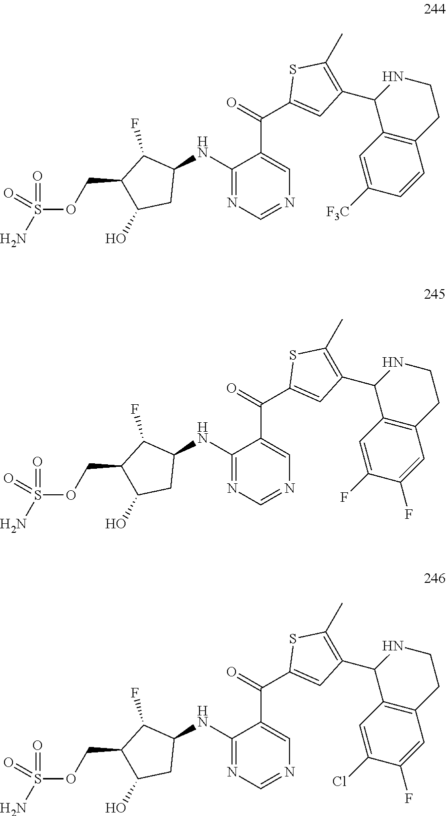

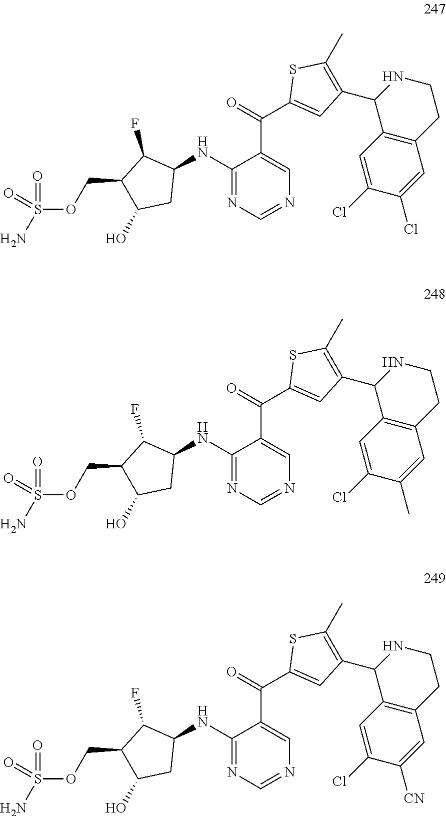

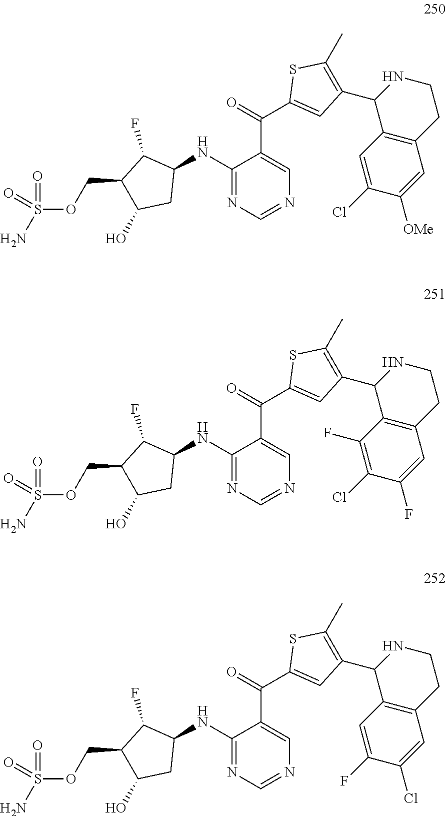

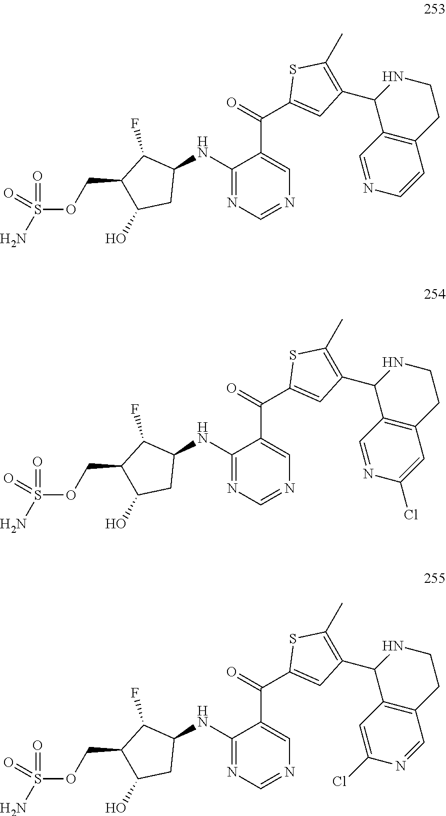

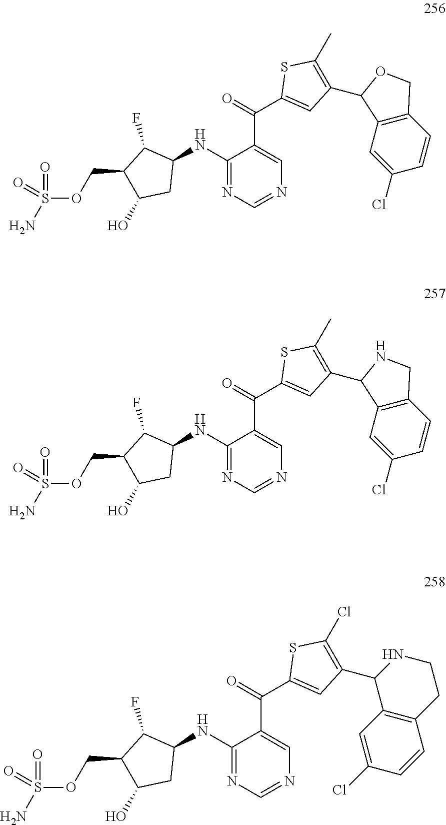

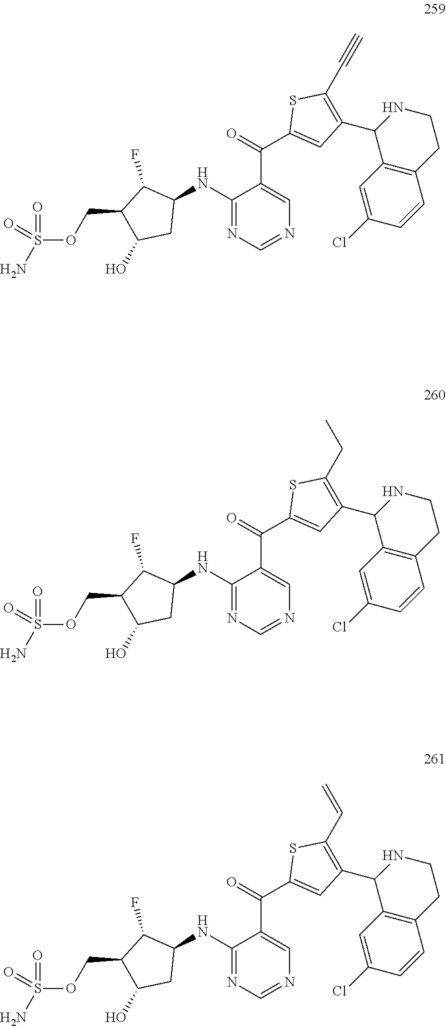

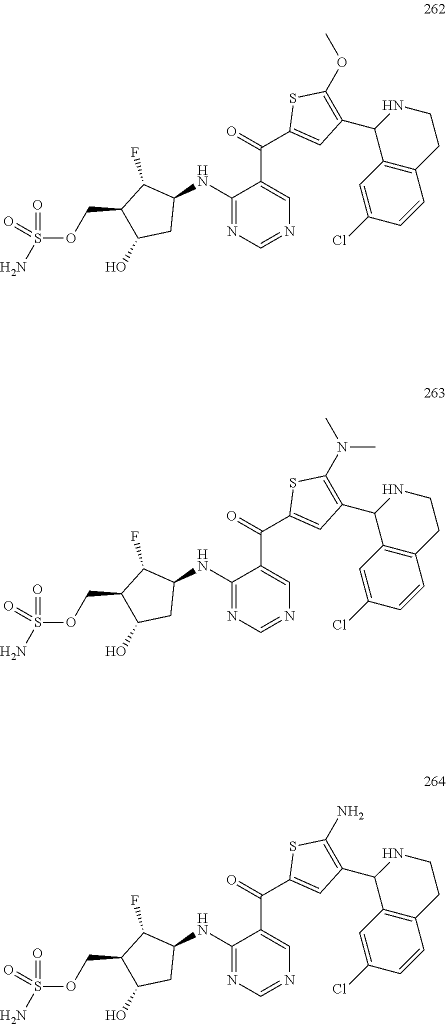

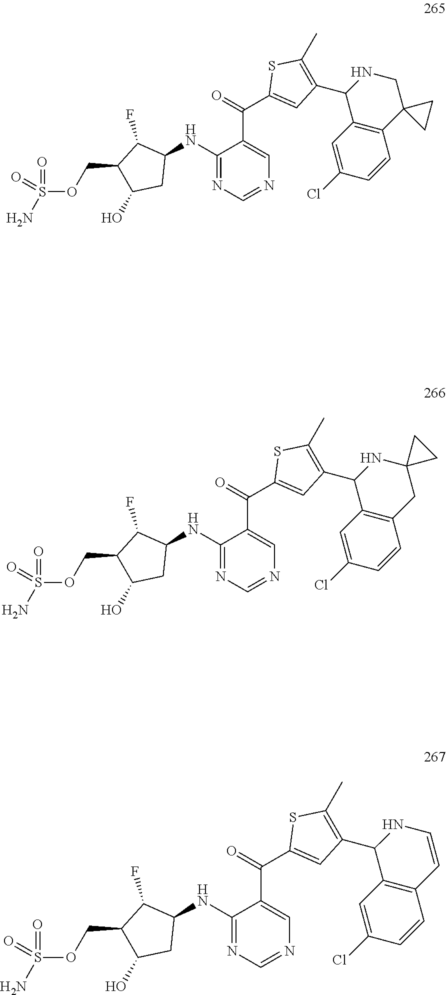

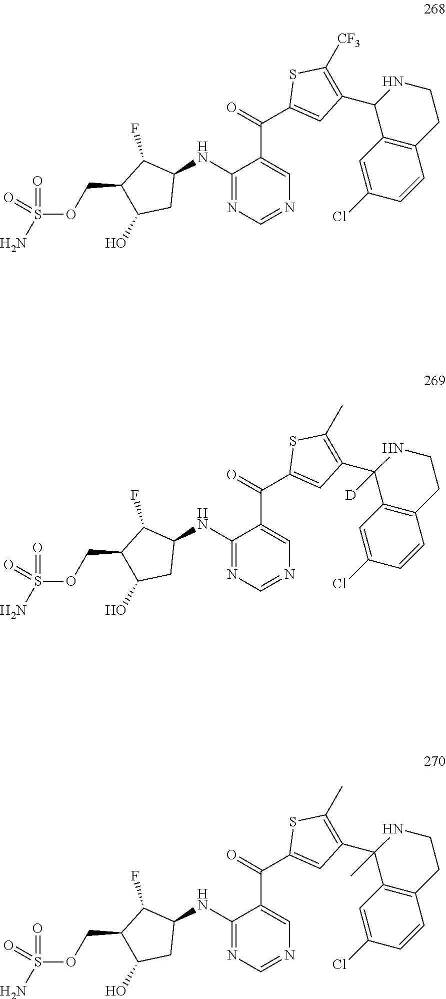

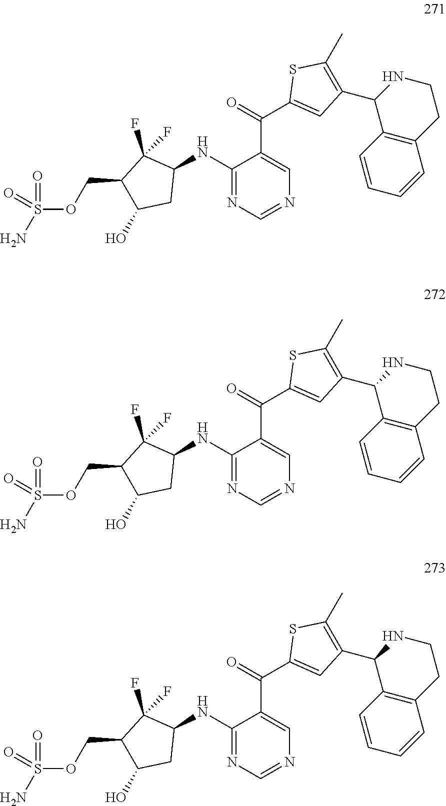

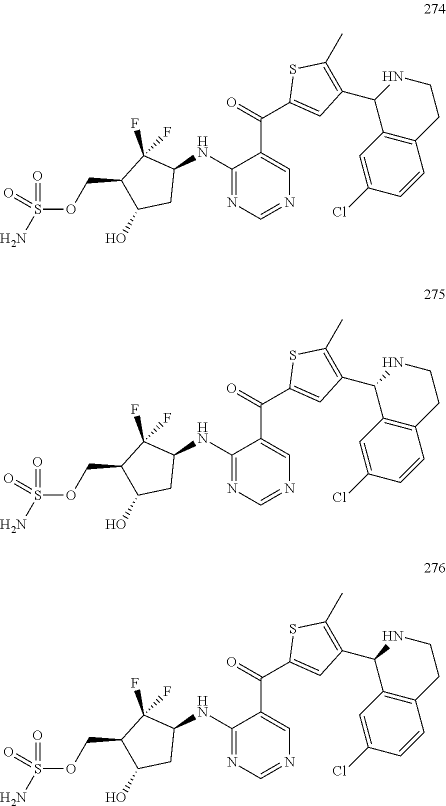

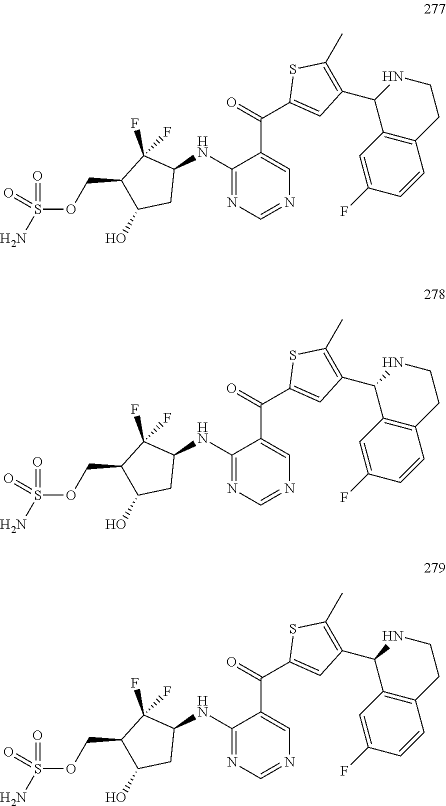

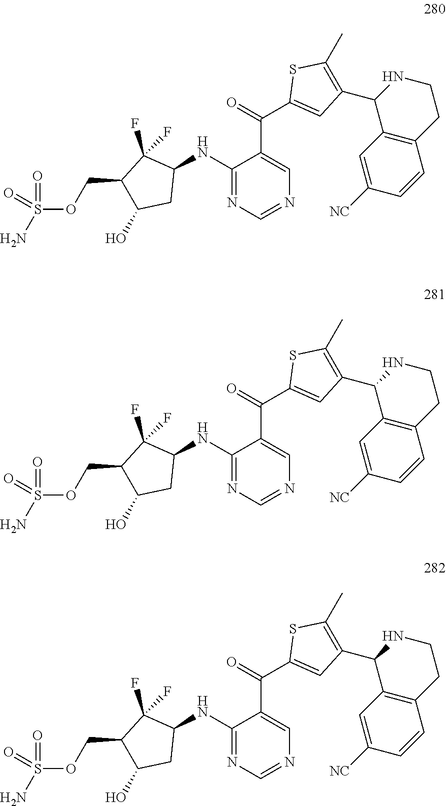

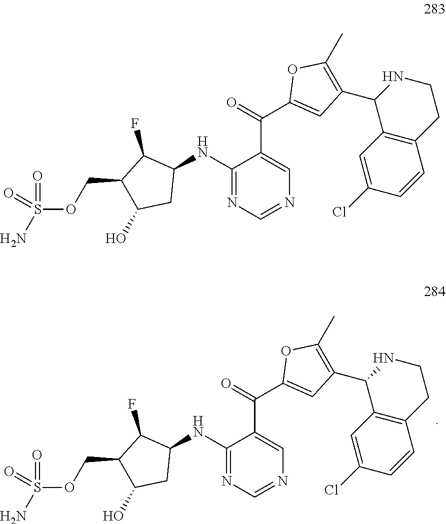

In another specific embodiment of the present disclosure, the compound of general formula (2) has one of the following structures:

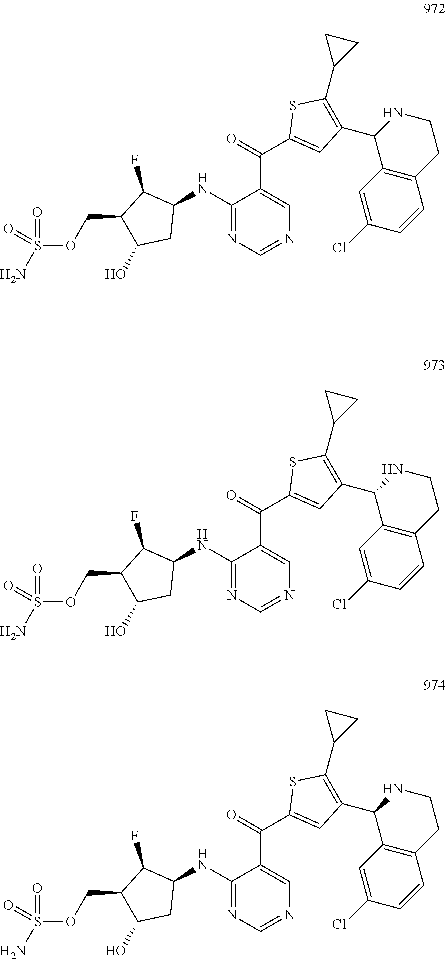

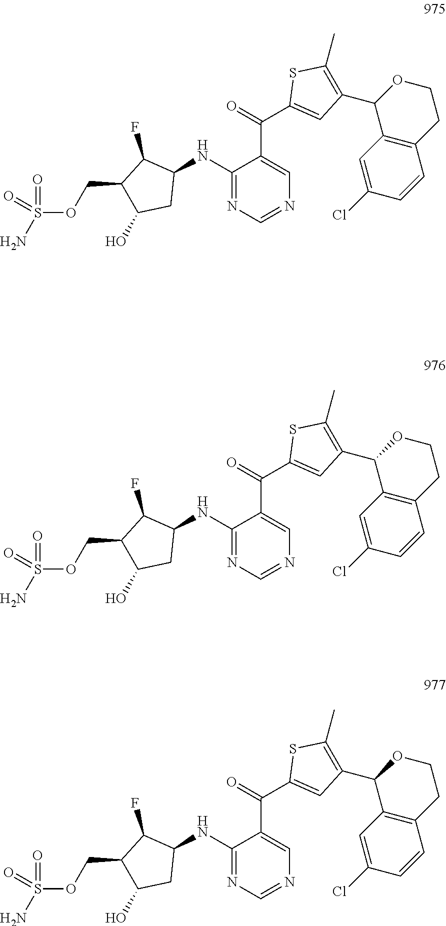

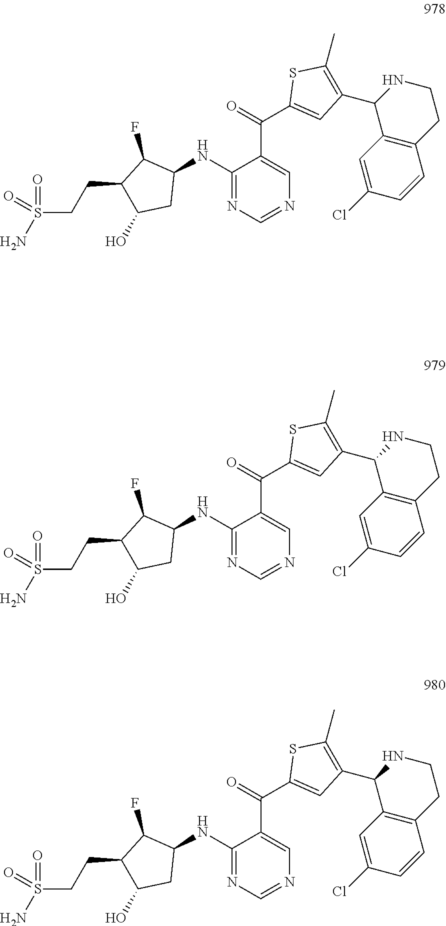

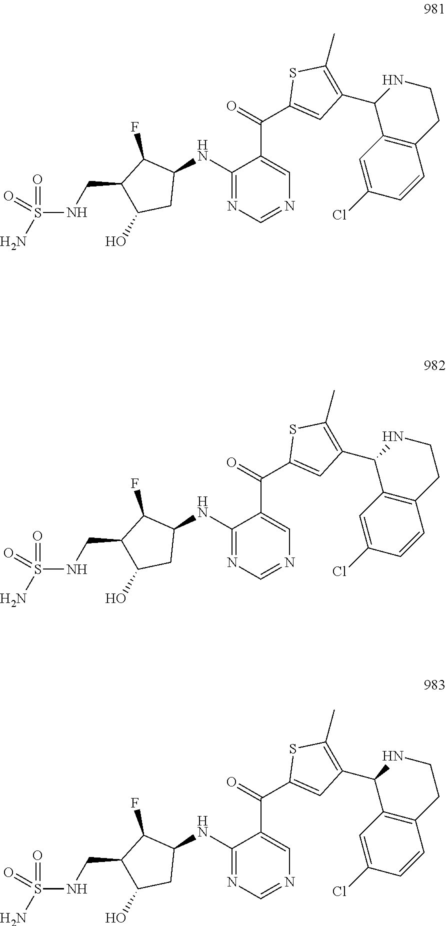

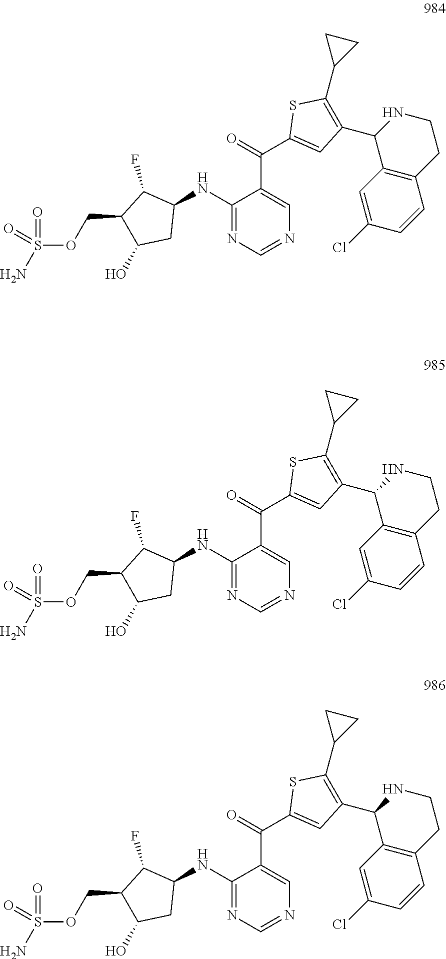

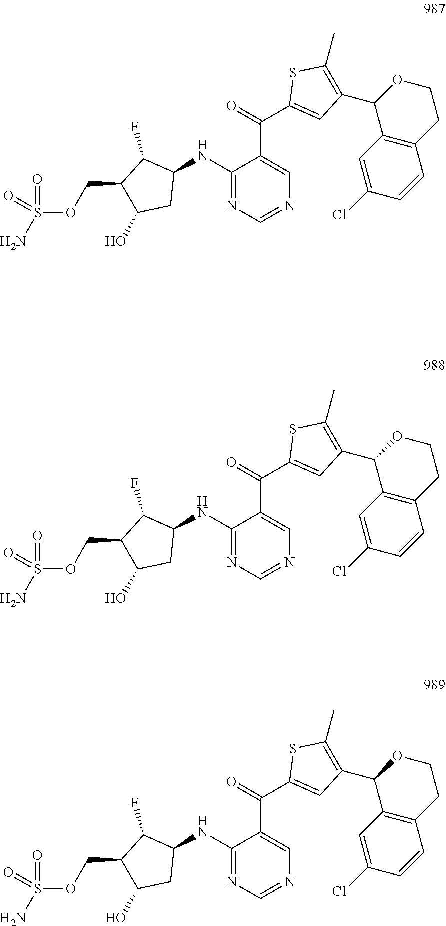

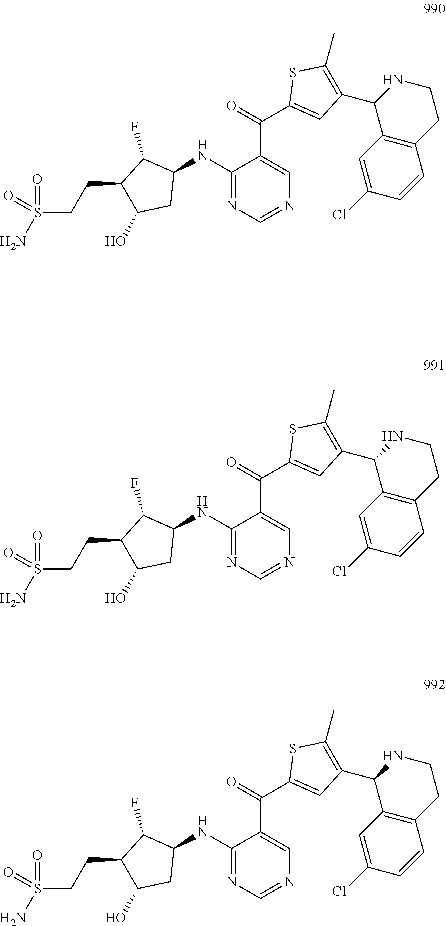

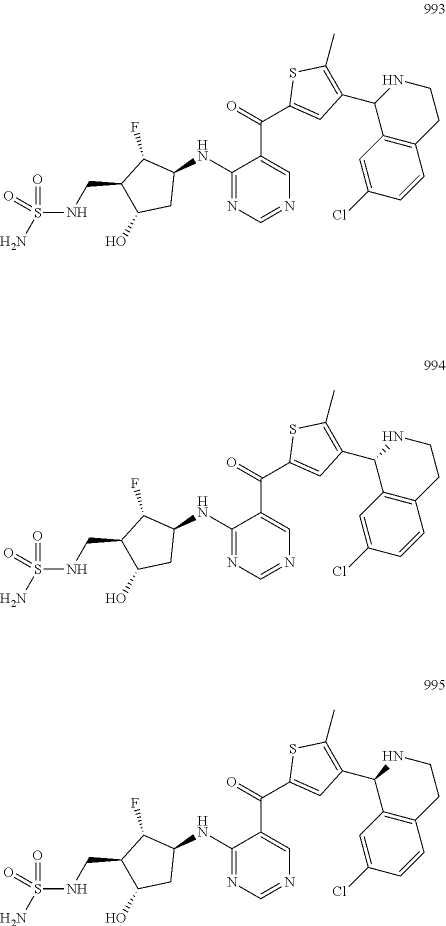

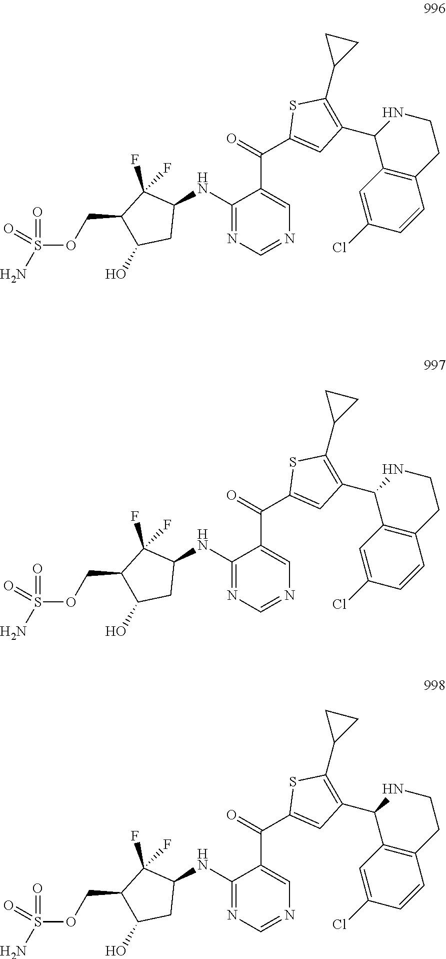

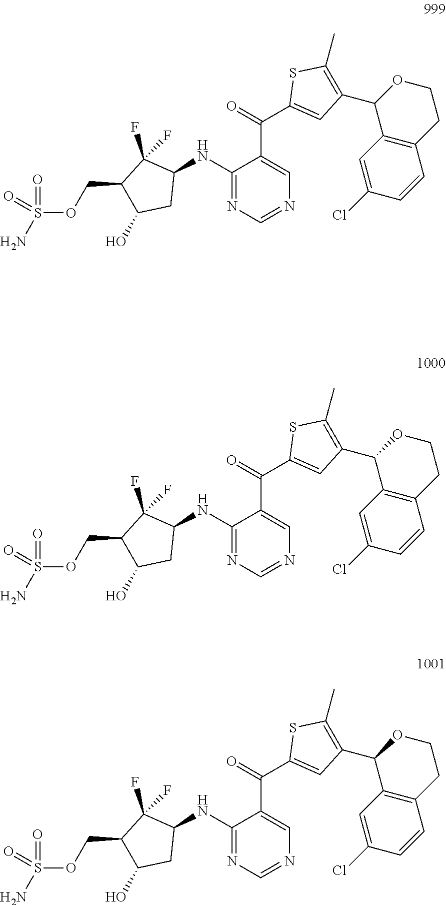

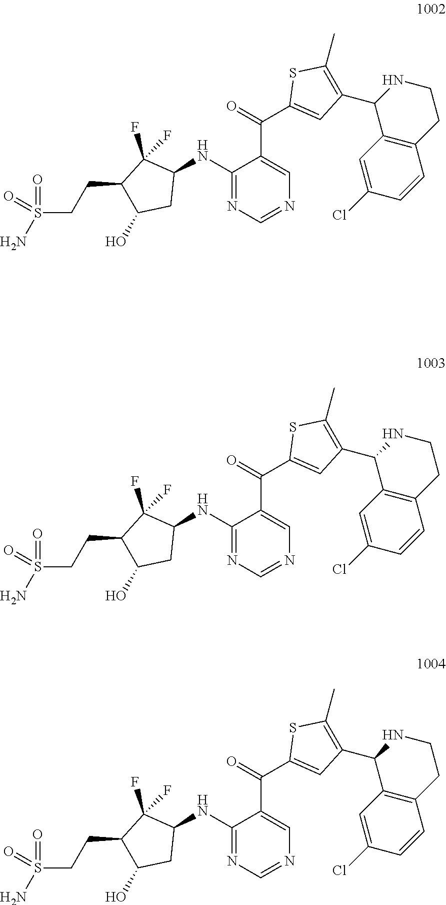

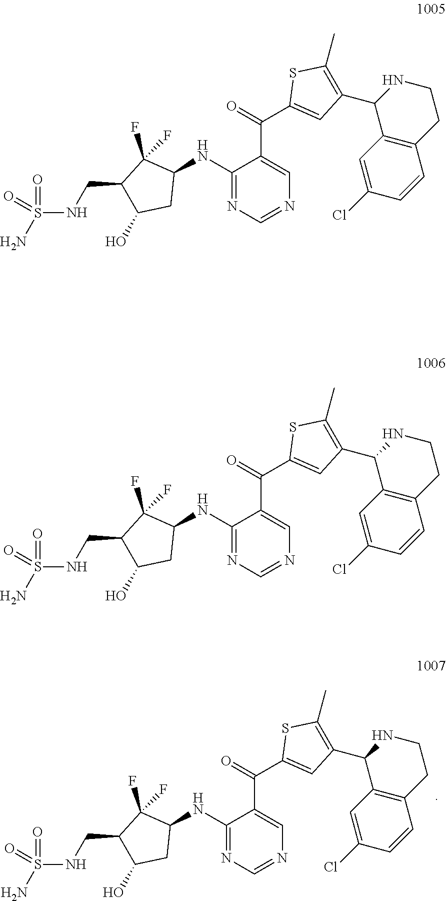

The present disclosure is further intended to provide a pharmaceutical composition comprising a pharmaceutically acceptable carrier, diluent and/or excipient, and the compound of general formula (1) or general formula (2) or the isomer, the crystalline form, the pharmaceutically acceptable salt, the hydrate or the solvate thereof of the present disclosure as an active ingredient.

The present disclosure is still further intended to provide use of the compound of general formula (1) or general formula (2) or the isomer, the crystalline form, the pharmaceutically acceptable salt, the hydrate or the solvate thereof of the present disclosure, or the pharmaceutical composition described above in the preparation of a medicament for treating, regulating or preventing a disease related to SAE protein, wherein the disease is preferably cancer, and the cancer is a hematologic cancer or a solid tumor.

The present disclosure is even further intended to provide a method for treating, regulating or preventing a disease related to SAE protein, comprising: administering to a subject a therapeutically effective amount of the compound of general formula (1) or general formula (2) or the isomer, the crystalline form, the pharmaceutically acceptable salt, the hydrate or the solvate thereof of the present disclosure, or the pharmaceutical composition described above.

It should be understood that both the aforementioned general description and the following detailed description of the present disclosure are exemplary and explanatory, and are intended to provide further explanation of the present disclosure claimed.

### Synthesis of Compounds

Methods for preparing the compounds disclosed herein are specifically described below, which, however, are not intended to limit the present disclosure in any way.

The compounds described above may be synthesized using standard synthetic techniques or well-known techniques in combination with the methods described herein. In addition, the solvents, temperatures, and other reaction conditions mentioned herein may vary. Starting materials for the synthesis of the compounds may be obtained synthetically or commercially. The compounds described herein and other related compounds with different substituents may be synthesized using well-known techniques and starting materials, including the methods found in March, ADVANCED ORGANIC CHEMISTRY, 4^{th} Ed., (Wiley 1992); Carey and Sundberg, ADVANCED ORGANIC CHEMISTRY, 4^{th} Ed., Vols. A and B (Plenum 2000,2001); and Green and Wuts, PROTECTIVE GROUPS IN ORGANIC SYNTHESIS, 3^{rd} Ed., (Wiley 1999). General methods for preparing the compounds may be altered by using appropriate reagents and conditions for introducing different groups into the molecular formulas provided herein.

In one aspect, the compounds described herein are prepared according to methods well known in the art. However, the conditions of the methods, such as reactants, solvents, bases, the amount of the compound used, reaction temperature, and time required for the reaction are not limited to the following explanation. The compounds of the present disclosure may also be conveniently prepared by optionally combining various synthetic methods described herein or known in the art, and such combinations may be easily determined by those skilled in the art to which the present disclosure pertains. In one aspect, the present disclosure further provides a method for preparing the compounds described herein, wherein the compound of general formula (1) or general formula (2) may be prepared by the following general reaction schemes 1-19.

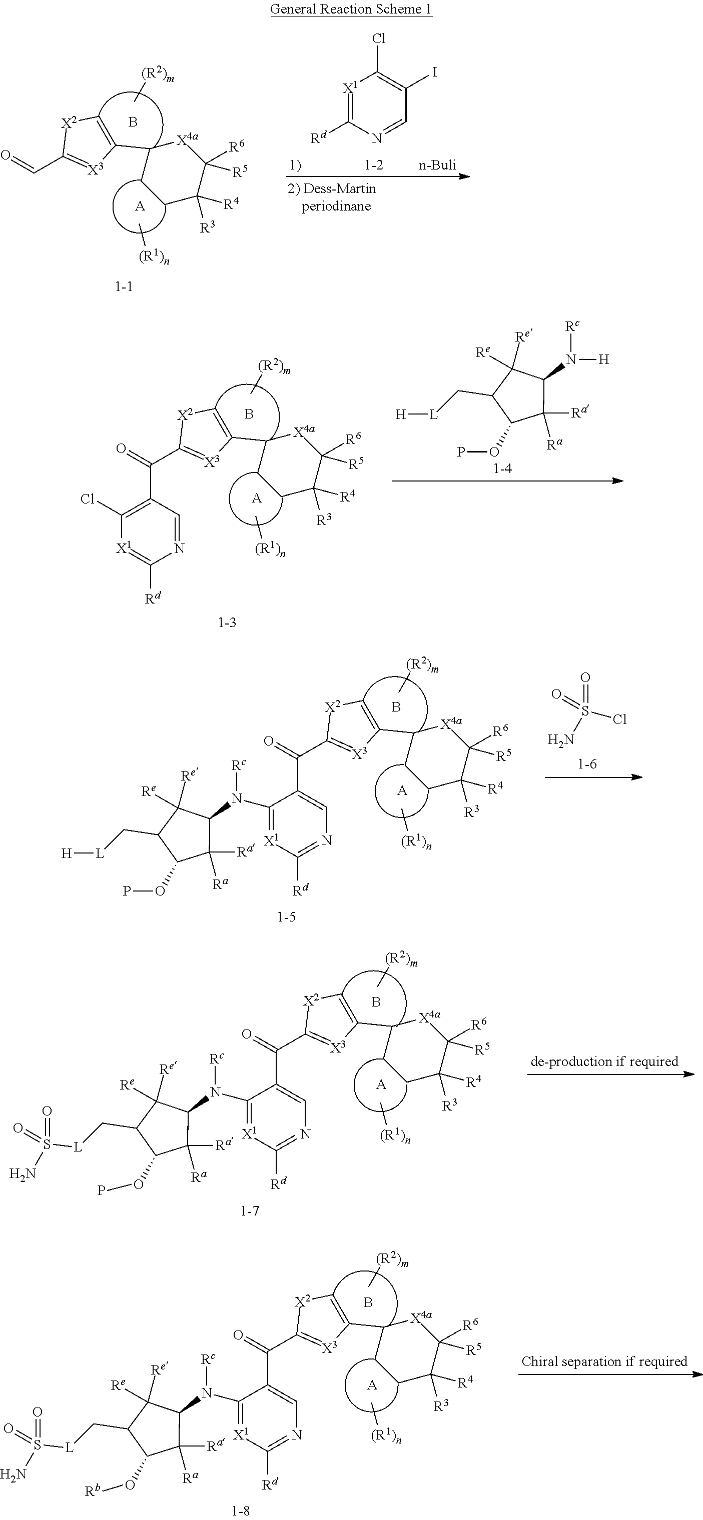

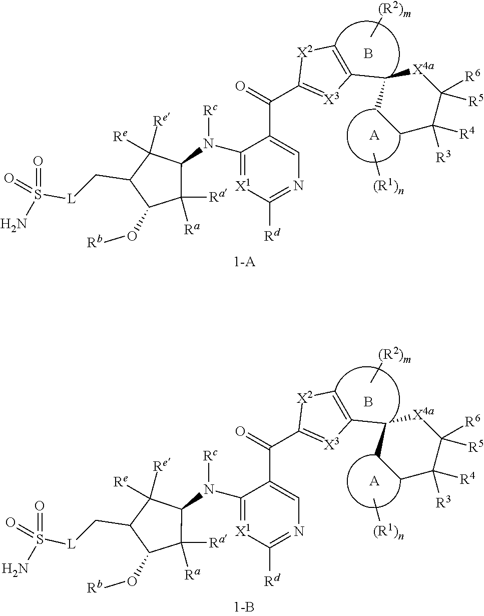

The compound of general formula (1) may be prepared according to general reaction scheme 1, wherein P is R^{b} or a hydroxyl-protecting group; ring A, ring B, R¹, R², R³, R⁴, R⁵, R⁶, R^{a}, R^{a′}, R^{b}, R^{c}, R^{d}, R^{e}, R^{e′}, X¹, X², X³, m, and n are as defined above; H represents hydrogen, N represents nitrogen, O represents oxygen, Cl represents chlorine, X^{4a} represents O or S, and L represents O or NH. As shown in general reaction scheme 1, compound 1-1 undergoes a substitution reaction with compound 1-2 to produce an alcohol intermediate, which is further oxidized to obtain ketone compound 1-3; compound 1-3 undergoes a substitution reaction with compound 1-4 to produce compound 1-5, and compound 1-5 undergoes a substitution reaction with compound 1-6 to produce compound 1-7; in some cases, compound 1-7 undergoes hydroxyl group deprotection to produce compound 1-8, and in some cases, compound 1-8 is subjected to chiral resolution to obtain optical isomers 1-A and 1-B.

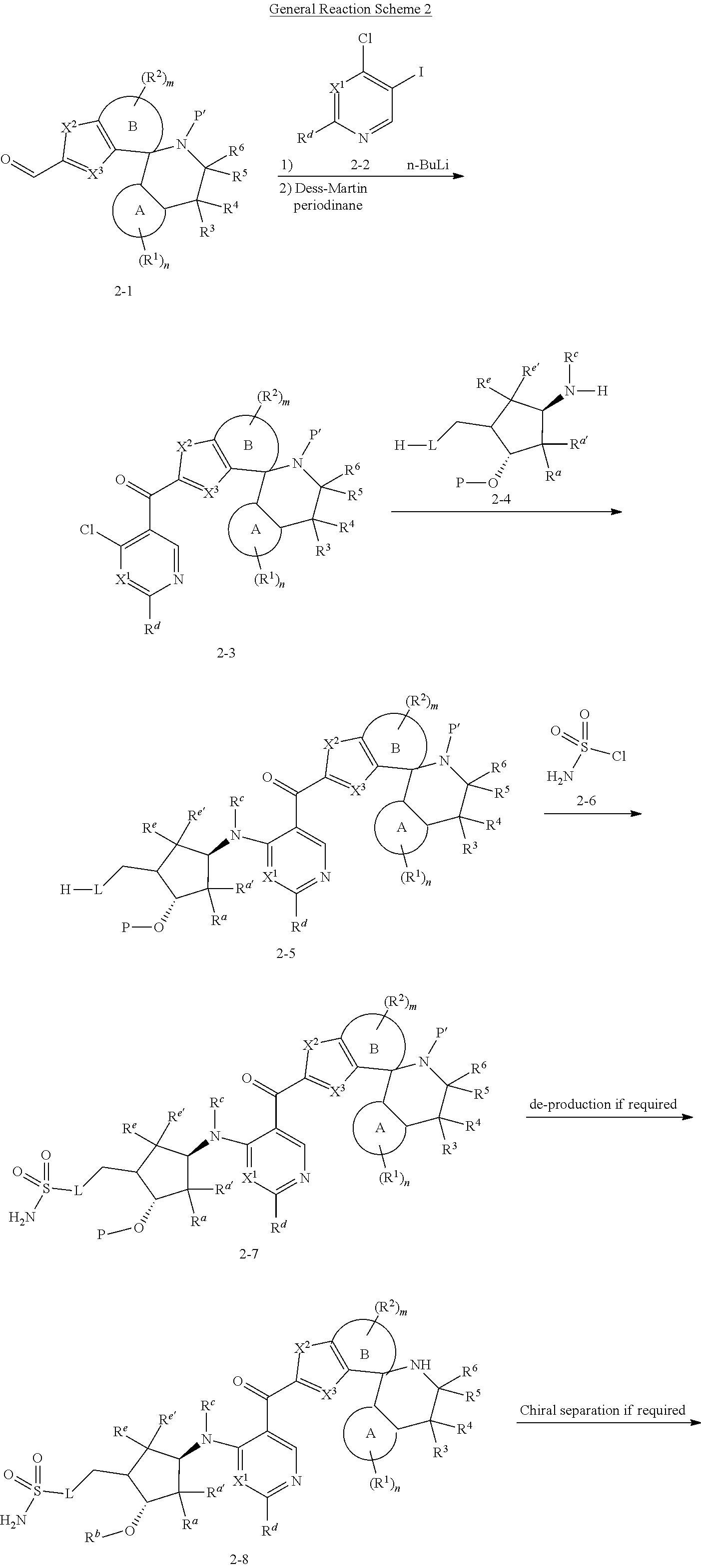

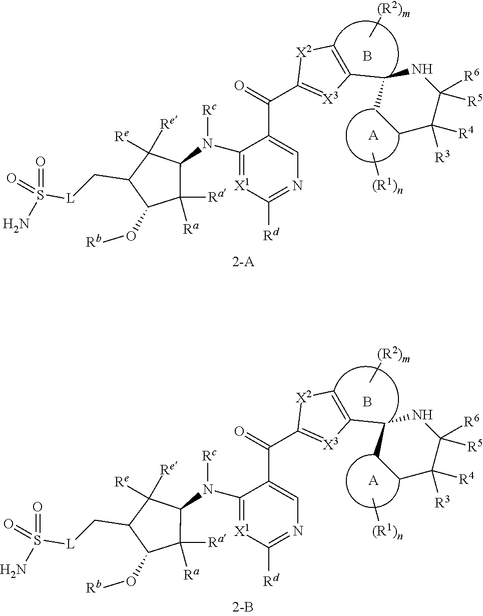

The compound of general formula (1) may be further prepared according to general reaction scheme 2, wherein P is R^{b} or a hydroxyl-protecting group, and P is an amino-protecting group; ring A, ring B, R¹, R², R³, R⁴, R⁵, R⁶, R^{a}, R^{a′}, R^{b}, R^{c}, R^{d}, R^{e}, R^{e′}, X¹, X², X³, m, and n are as defined above; H represents hydrogen, N represents nitrogen, O represents oxygen, Cl represents chlorine, and L represents O or NH. As shown in general reaction scheme 1, compound 2-1 undergoes a substitution reaction with compound 2-2 to produce an alcohol intermediate, which is further oxidized to obtain ketone compound 2-3; compound 2-3 undergoes a substitution reaction with compound 2-4 to produce compound 2-5, and compound 2-5 undergoes a substitution reaction with compound 2-6 to produce compound 2-7; in some cases, compound 2-7 undergoes deprotection to produce compound 2-8, and in some cases, compound 2-8 is subjected to chiral resolution to obtain optical isomers 2-A and 2-B.

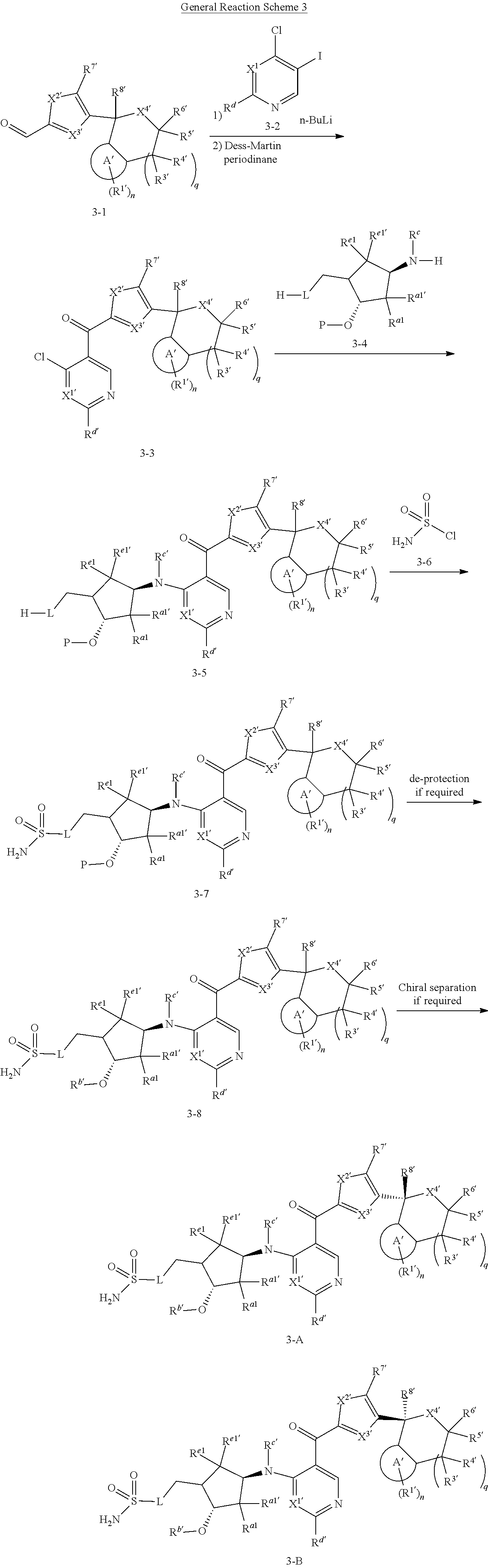

The compound of general formula (2) may be prepared according to general reaction scheme 3, wherein P is R^{b′} or a hydroxyl-protecting group; ring A′, R^{1′}, R^{3′}, R^{4′}, R^{5′}, R^{6′}, R^{7′}, R^{8′}, R^{a1}, R^{a1′}, R^{b′}, R^{c′}, R^{d′}, R^{e1}, R^{e1′}, X^{1′}, X^{2′}, X^{3′}, n, and q are as defined above; H represents hydrogen, N represents nitrogen, O represents oxygen, Cl represents chlorine, X^{4a} represents O or S and L represents O or NH. As shown in general reaction scheme 3, compound 3-1 undergoes a substitution reaction with compound 3-2 to produce an alcohol intermediate, which is further oxidized to obtain ketone compound 3-3; compound 3-3 undergoes a substitution reaction with compound 3-4 to produce compound 3-5, and compound 3-5 undergoes a substitution reaction with compound 3-6 to produce compound 3-7; in some cases, compound 3-7 undergoes hydroxyl group deprotection to produce compound 3-8, and in some cases, compound 3-8 is subjected to chiral resolution to obtain optical isomers 3-A and 3-1B.

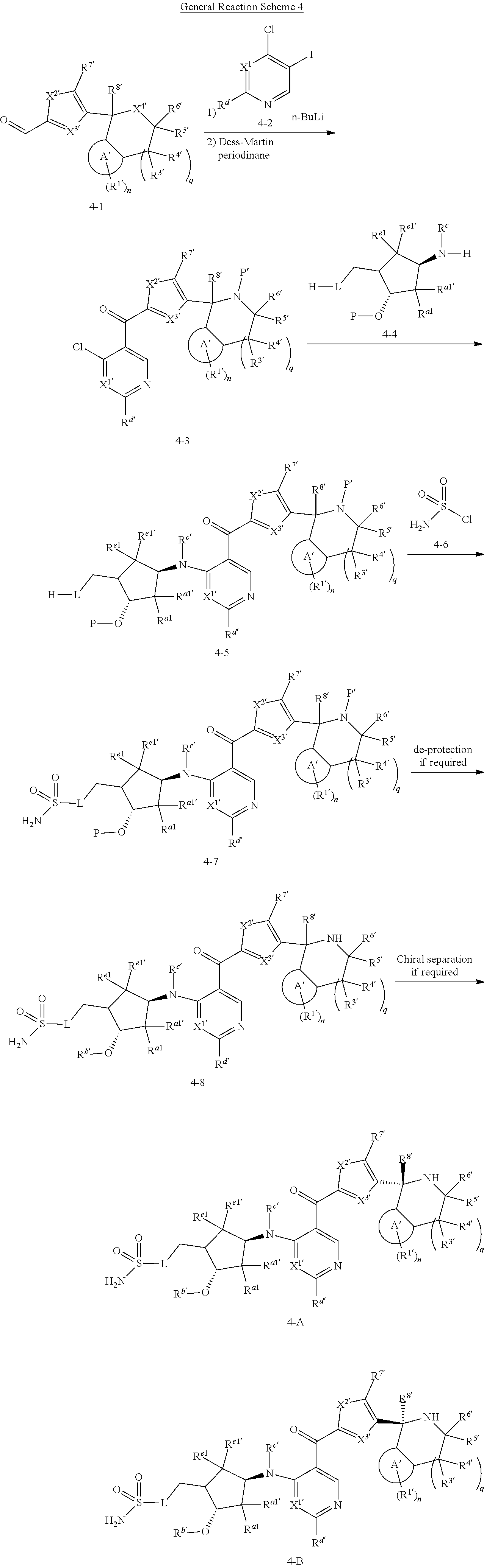

The compound of general formula (2) may be further prepared according to general reaction scheme 4, wherein P is R^{b′} or a hydroxyl-protecting group, and P′ is an amino-protecting group; ring A′, R^{1′}, R^{3′}, R^{4′}, R^{5′}, R^{6′}, R^{7′}, R^{8′}, R^{a1}, R^{a1′}, R^{b′}, R^{c′}, R^{d′}, R^{e1}, R^{e1′}, X^{1′}, X^{2′}, X^{3′}, n, and q are as defined above; H represents hydrogen, N represents nitrogen, O represents oxygen, Cl represents chlorine, X^{4a} represents O or S, and L represents O or NH. As shown in general reaction scheme 4, compound 4-1 undergoes a substitution reaction with compound 4-2 to produce an alcohol intermediate, which is further oxidized to obtain ketone compound 4-3; compound 4-3 undergoes a substitution reaction with compound 4-4 to produce compound 4-5, and compound 4-5 undergoes a substitution reaction with compound 4-6 to produce compound 4-7; in some cases, compound 4-7 undergoes hydroxyl group deprotection to produce compound 4-8, and in some cases, compound 4-8 is subjected to chiral resolution to obtain optical isomers 4-A and 4-B.

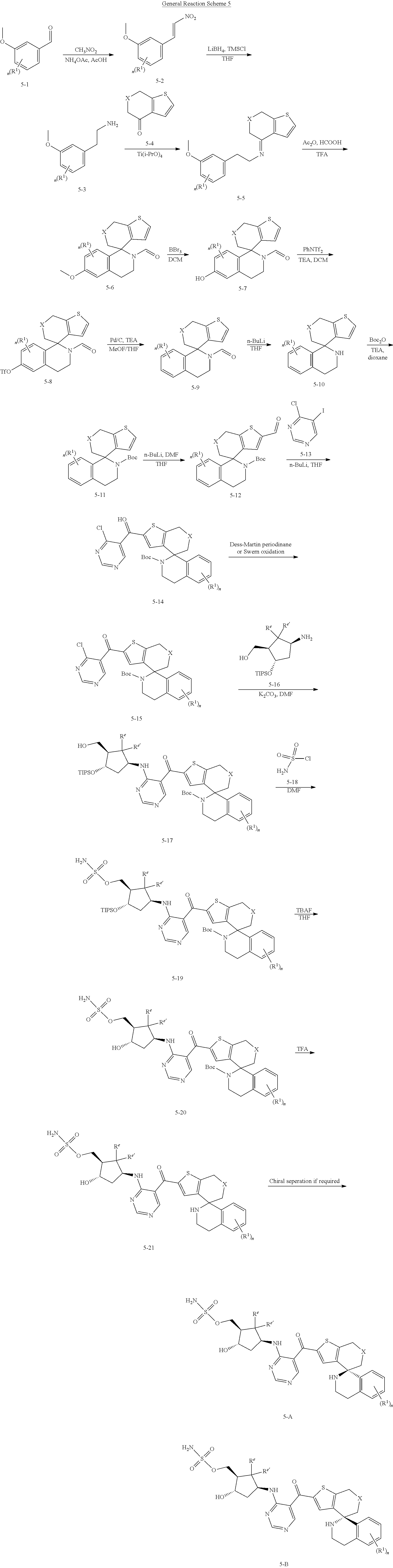

Embodiments of the compound of general formula (1) may be prepared according to general reaction scheme 5, wherein R¹, R^{e}, R^{e′}, and n are as defined above, X represents O, S, or CH₂, H represents hydrogen, N represents nitrogen, O represents oxygen, and Cl represents chlorine. As shown in general reaction scheme 5, compound 5-1 undergoes a condensation reaction with nitromethane to produce compound 5-2, and compound 5-2 is reduced to obtain amine 5-3; amine 5-3 undergoes a condensation reaction with ketone 5-4 to obtain compound 5-5, and compound 5-5 undergoes a cyclization reaction under an acidic condition to produce compound 5-6; compound 5-6 is demethylated to obtain compound 5-7, and compound 5-7 reacts with PhNTf₂ to produce compound 5-8; compound 5-8 is reduced to obtain compound 5-9, and compound 5-9 undergoes formyl group removal to obtain compound 5-10; compound 5-10 is protected by (Boc)₂O to obtain compound 5-11, and compound 5-11 reacts with DMF under the condition of n-butyllithium to obtain aldehyde 5-12; aldehyde 5-12 undergoes an addition reaction with compound 5-13 under the condition of n-butyllithium to obtain compound 5-14, and compound 5-14 is subjected to Dess-Martin oxidation or Swern oxidation to obtain ketone 5-15; ketone 5-15 undergoes a substitution reaction with compound 5-16 under an alkaline condition to produce compound 5-17, and compound 5-17 reacts with compound 5-18 to produce compound 5-19; compound 5-19 undergoes deprotection by TBAF to obtain compound 5-20, and compound 5-20 undergoes amino group deprotection under an acidic condition to obtain compound 5-21; and in some cases, compound 5-21 is subjected to chiral resolution to obtain optical isomers 5-A and 5-B.

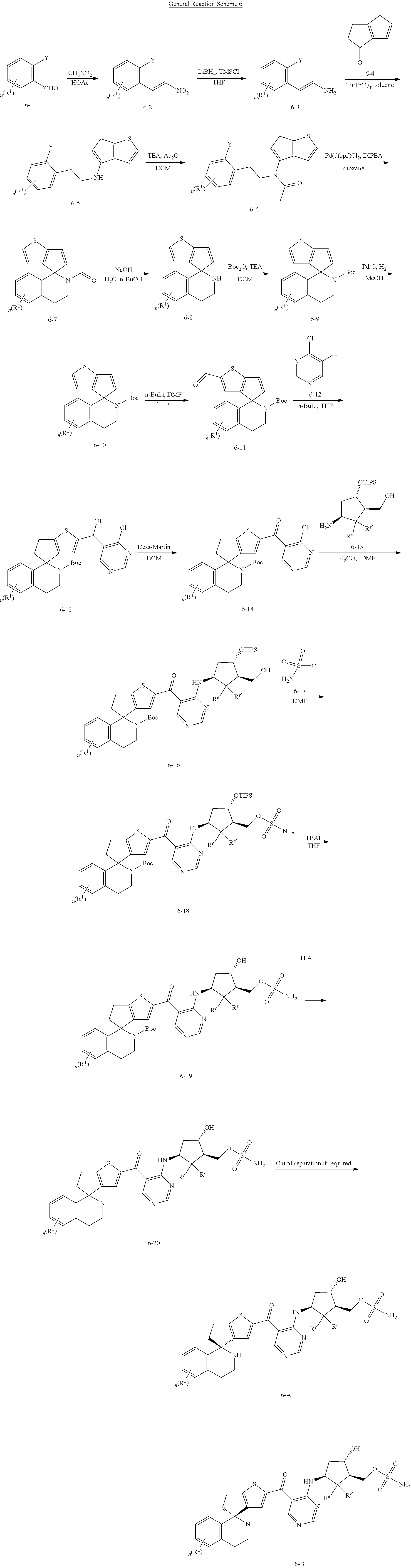

Embodiments of the compound of general formula (1) may be prepared according to general reaction scheme 6, wherein R¹, R^{e}, R^{e′}, and n are as defined above, Y represents Br or I, H represents hydrogen, N represents nitrogen, O represents oxygen, and Cl represents chlorine. As shown in general reaction scheme 6, compound 6-1 undergoes a condensation reaction with nitromethane to produce compound 6-2, and compound 6-2 is reduced to obtain amine 6-3; amine 6-3 undergoes a condensation reaction with ketone 6-4 to obtain compound 6-5, and compound 6-5 reacts with acetic anhydride to produce compound 6-6; compound 6-6 undergoes a cyclization reaction under the catalysis of Pd to produce compound 6-7, and compound 6-7 undergoes deacetylation to obtain compound 6-8; compound 6-8 is protected by (Boc)₂O to obtain compound 6-9, and compound 6-9 undergoes a hydrogenation reaction to produce compound 6-10; compound 6-10 reacts with DMF under the condition of n-butyllithium to obtain aldehyde 6-11, and aldehyde 6-11 undergoes an addition reaction with compound 6-12 under the condition of n-butyllithium to obtain compound 6-13; compound 6-13 is subjected to Dess-Martin oxidation to obtain ketone 6-14, and ketone 6-14 undergoes a substitution reaction with compound 6-15 under an alkaline condition to produce compound 6-16; compound 6-16 reacts with compound 6-17 to produce compound 6-18, and compound 6-18 undergoes deprotection by TBAF to obtain compound 6-19; compound 6-19 undergoes amino group deprotection under an acidic condition to obtain compound 6-20, and in some cases, compound 6-20 is subjected to chiral resolution to obtain optical isomers 6-A and 6-B.

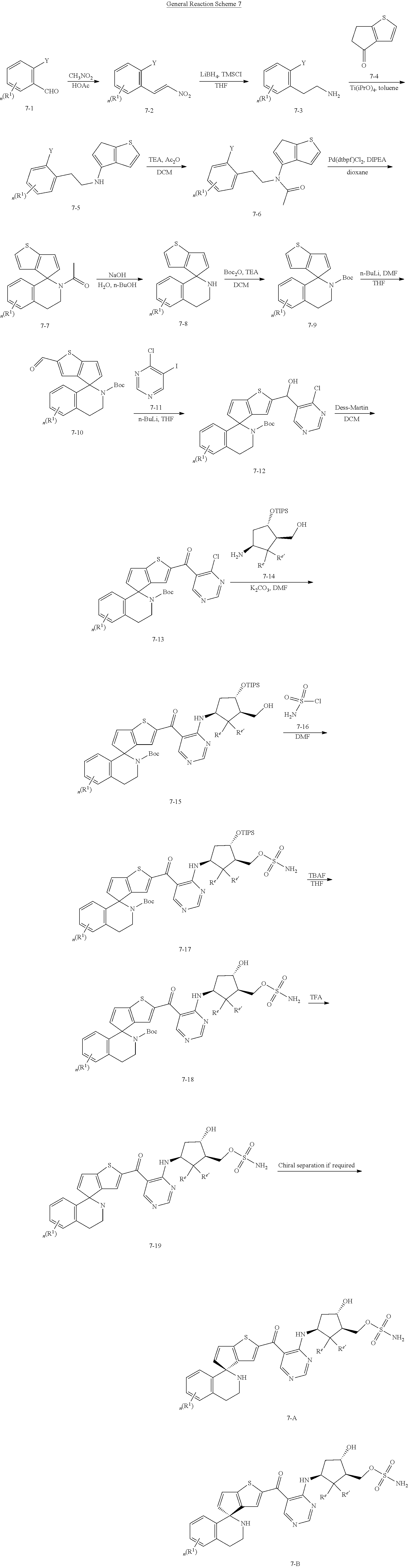

Embodiments of the compound of general formula (1) may be prepared according to general reaction scheme 7, wherein R¹, R^{e}, R^{e′}, and n are as defined above, Y represents Br or I, H represents hydrogen, N represents nitrogen, O represents oxygen, and Cl represents chlorine. As shown in general reaction scheme 7, compound 7-1 undergoes a condensation reaction with nitromethane to produce compound 7-2, and compound 7-2 is reduced to obtain amine 7-3; amine 7-3 undergoes a condensation reaction with ketone 7-4 to obtain compound 7-5, and compound 7-5 reacts with acetic anhydride to produce compound 7-6; compound 7-6 undergoes a cyclization reaction under the catalysis of Pd to produce compound 7-7, and compound 7-7 undergoes deacetylation to obtain compound 7-8; compound 7-8 is protected by (Boc)₂O to obtain compound 7-9, and compound 7-9 reacts with DMF under the condition of n-butyllithium to obtain aldehyde 7-10; aldehyde 7-10 undergoes an addition reaction with compound 7-11 under the condition of n-butyllithium to obtain compound 7-12, and compound 7-12 is subjected to Dess-Martin oxidation to obtain ketone 7-13; ketone 7-13 undergoes a substitution reaction with compound 7-14 under an alkaline condition to produce compound 7-15, and compound 7-15 reacts with compound 7-16 to produce compound 7-17; compound 7-17 undergoes deprotection by TBAF to obtain compound 7-18, and compound 7-18 undergoes amino group deprotection under an acidic condition to obtain compound 7-19; and in some cases, compound 7-19 is subjected to chiral resolution to obtain optical isomers 7-A and 7-B.

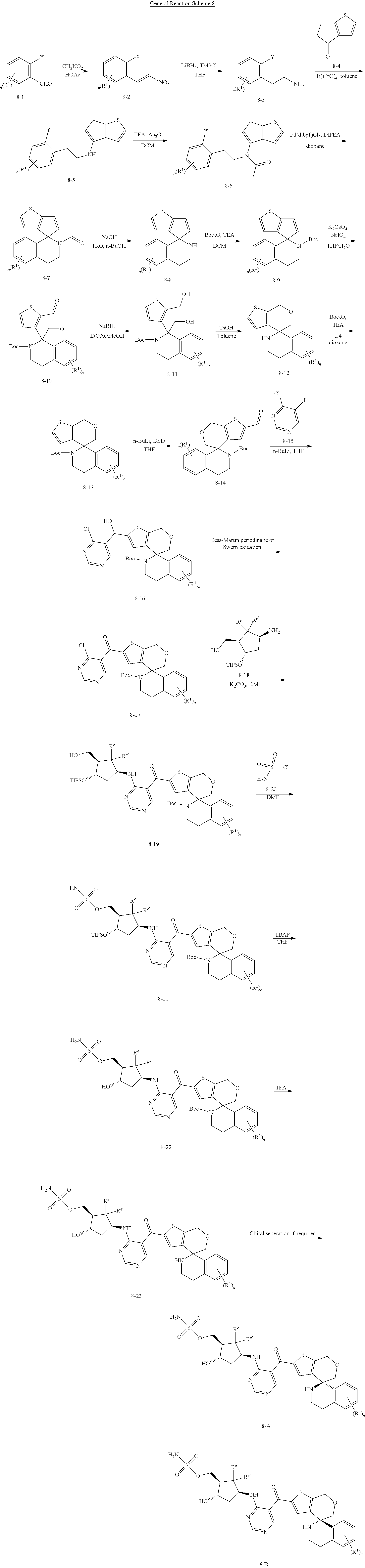

Embodiments of the compound of general formula (1) may be prepared according to general reaction scheme 8, wherein R¹, R^{e}, R^{e′}, and n are as defined above, Y represents Br or I, H represents hydrogen, N represents nitrogen, O represents oxygen, and Cl represents chlorine. As shown in general reaction scheme 8, compound 8-1 undergoes a condensation reaction with nitromethane to produce compound 8-2, and compound 8-2 is reduced to obtain amine 8-3; amine 8-3 undergoes a condensation reaction with ketone 8-4 to obtain compound 8-5, and compound 8-5 reacts with acetic anhydride to produce compound 8-6; compound 8-6 undergoes a cyclization reaction under the catalysis of Pd to produce compound 8-7, and compound 8-7 undergoes deacetylation to obtain compound 8-8; compound 8-8 is protected by (Boc)₂O to obtain compound 8-9, and compound 8-9 undergoes an oxidation reaction under the condition of potassium osmate and sodium periodate to obtain dialdehyde 8-10; dialdehyde 8-10 is reduced with sodium borohydride to obtain diol 8-11, and diol 8-11 undergoes a cyclization reaction under an acidic condition to obtain compound 8-12; compound 8-12 is protected by (Boc)₂O to obtain compound 8-13, and compound 8-13 reacts with DMF under the condition of n-butyllithium to obtain aldehyde 8-14; aldehyde 8-14 undergoes an addition reaction with compound 8-15 under the condition of n-butyllithium to obtain compound 8-16, and compound 8-18 is subjected to Dess-Martin oxidation or Swern oxidation to obtain ketone 8-17; ketone 8-17 undergoes a substitution reaction with compound 8-18 under an alkaline condition to produce compound 8-19, and compound 8-19 reacts with compound 8-20 to produce compound 8-21; compound 8-21 undergoes deprotection by TBAF to obtain compound 8-22, and compound 8-22 undergoes amino group deprotection under an acidic condition to obtain compound 8-23; and in some cases, compound 8-23 is subjected to chiral resolution to obtain optical isomers 8-A and 8-B.

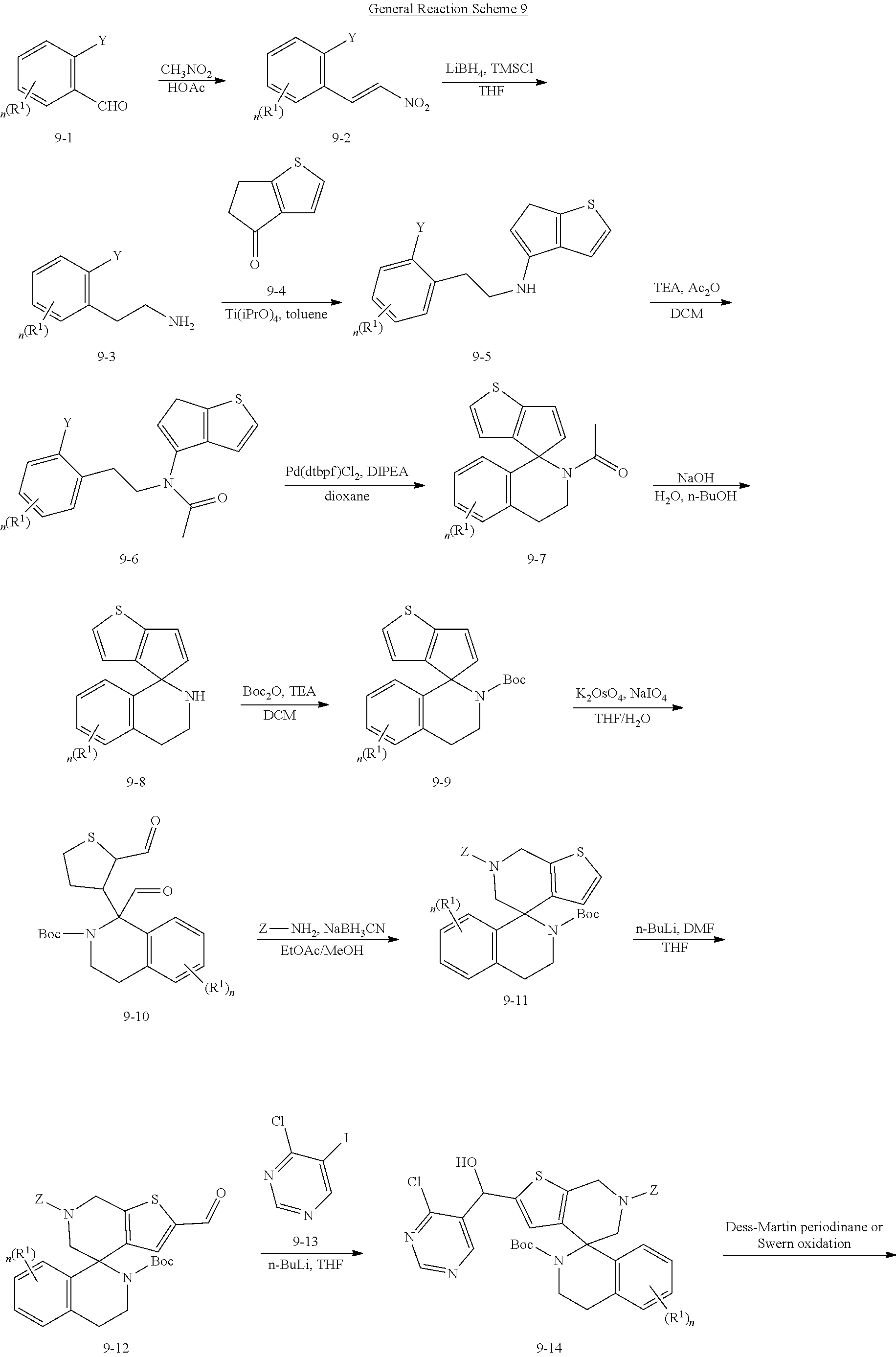

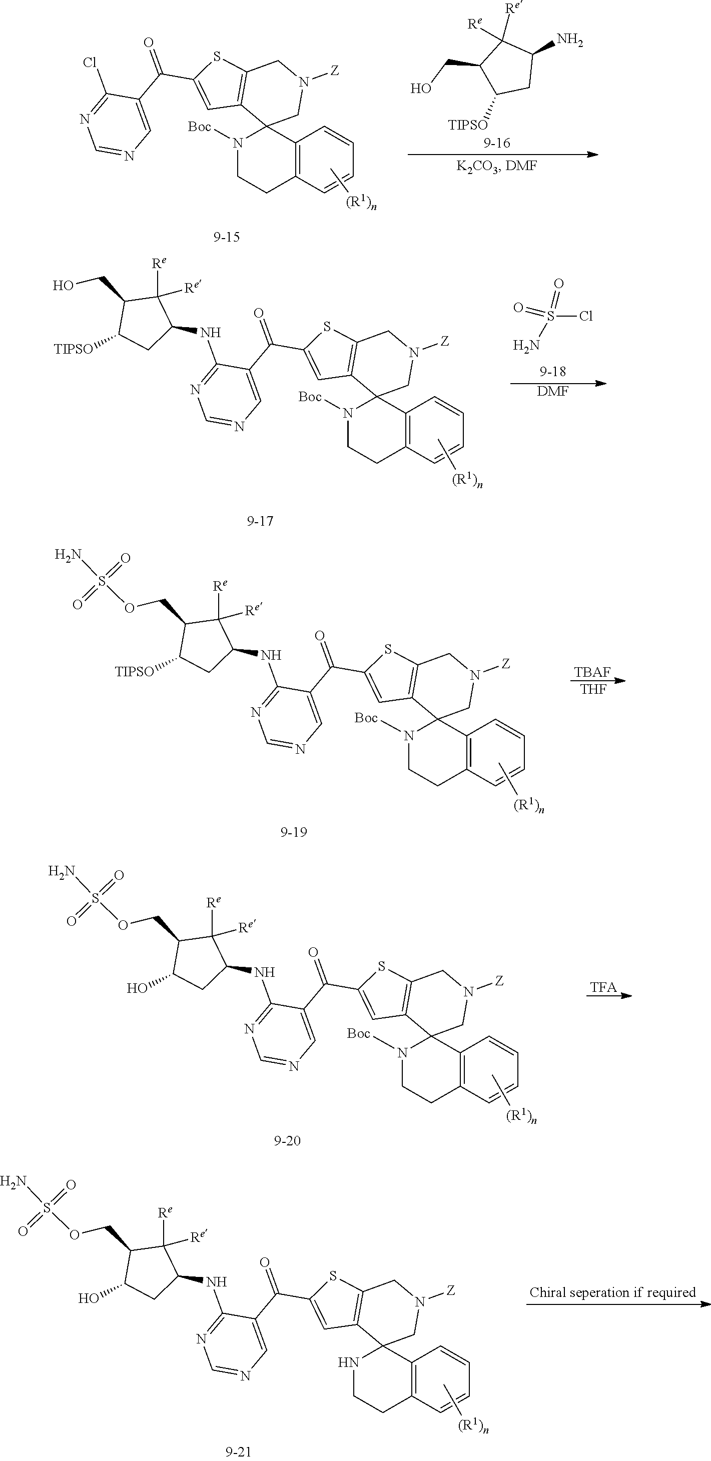

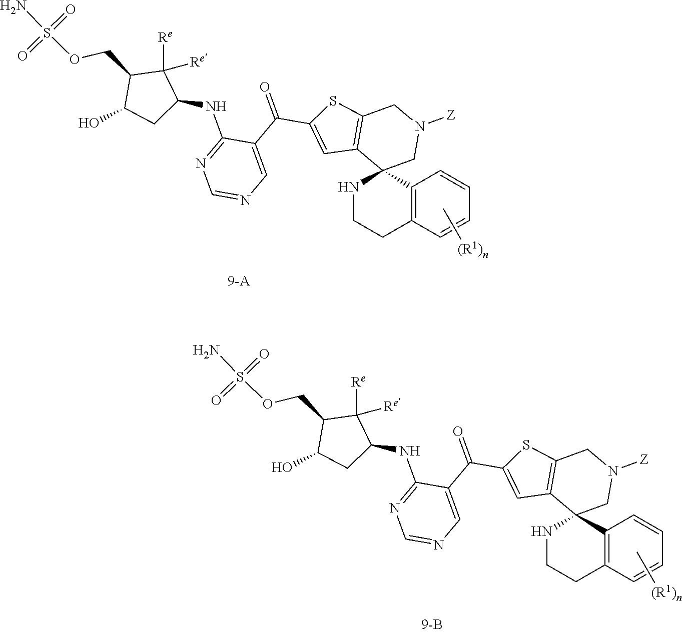

Embodiments of the compound of general formula (1) may be prepared according to general reaction scheme 9, wherein R¹, R^{e}, R^{e′}, and n are as defined above, Y represents Br or I, Z represents —H, —OH, (C1-C6) alkyl, (C1-C6) haloalkyl, (C1-C6) alkoxy, (C2-C6) alkenyl, (C2-C6) alkynyl, or (C3-C8) cycloalkyl, H represents hydrogen, N represents nitrogen, O represents oxygen, and Cl represents chlorine. As shown in general reaction scheme 9, compound 9-1 undergoes a condensation reaction with nitromethane to produce compound 9-2, and compound 9-2 is reduced to obtain amine 9-3; amine 9-3 undergoes a condensation reaction with ketone 9-4 to obtain compound 9-5, and compound 9-5 reacts with acetic anhydride to produce compound 9-6; compound 9-6 undergoes a cyclization reaction under the catalysis of Pd to produce compound 9-7, and compound 9-7 undergoes deacetylation to obtain compound 9-8; compound 9-8 is protected by (Boc)₂O to obtain compound 9-9, and compound 9-9 undergoes an oxidation reaction under the condition of potassium osmate and sodium periodate to obtain dialdehyde 9-10; dialdehyde 9-10 undergoes reductive amination to obtain amine 9-11, and compound 9-11 reacts with DMF under the condition of n-butyllithium to obtain aldehyde 9-12; aldehyde 9-12 undergoes an addition reaction with compound 9-13 under the condition of n-butyllithium to obtain compound 9-14, and compound 9-14 is subjected to Dess-Martin oxidation or Swern oxidation to obtain ketone 9-15; ketone 9-15 undergoes a substitution reaction with compound 9-16 under an alkaline condition to produce compound 9-17, and compound 9-17 reacts with compound 9-18 to produce compound 9-19; compound 9-19 undergoes deprotection by TBAF to obtain compound 9-20, and compound 9-20 undergoes amino group deprotection under an acidic condition to obtain compound 9-21; and in some cases, compound 9-21 is subjected to chiral resolution to obtain optical isomers 9-A and 9-B.

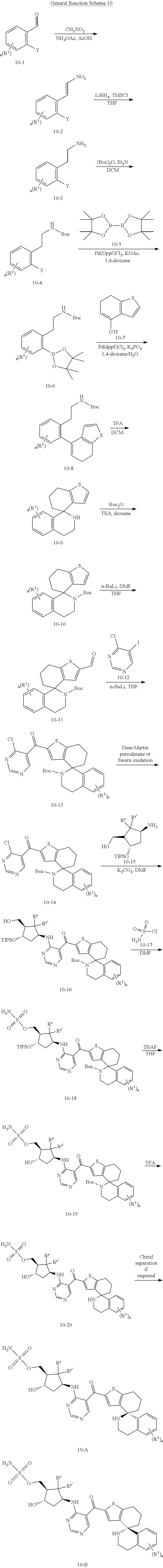

Embodiments of the compound of general formula (1) may be prepared according to general reaction scheme 10, wherein R¹, R^{e}, R^{e′}, and n are as defined above, Y represents Br, I, or OTf, H represents hydrogen, N represents nitrogen, O represents oxygen, and Cl represents chlorine. As shown in general reaction scheme 10, compound 10-1 undergoes a condensation reaction with nitromethane to produce compound 10-2, and compound 10-2 is reduced to obtain amine 10-3; compound 10-3 is protected by (Boc)₂O to obtain compound 10-4; compound 10-4 undergoes a coupling reaction with compound 10-5 under the catalysis of Pd to obtain compound 10-6; compound 10-6 undergoes a coupling reaction with compound 10-7 under the catalysis of Pd to obtain compound 10-8; compound 10-8 undergoes a cyclization reaction under the condition of trifluoroacetic acid and dichloromethane to produce compound 10-9, and compound 10-9 is protected by (Boc)₂O to obtain compound 10-10; compound 10-10 reacts with DMF under the condition of n-butyllithium to obtain aldehyde 10-11, and aldehyde 10-11 undergoes an addition reaction with compound 10-12 under the condition of n-butyllithium to obtain compound 10-13; compound 10-13 is subjected to Dess-Martin oxidation or Swern oxidation to obtain ketone 10-14, and ketone 10-14 undergoes a substitution reaction with compound 10-15 under an alkaline condition to produce compound 10-16; compound 10-16 reacts with compound 10-17 to produce compound 10-18, and compound 10-18 undergoes deprotection by TBAF to obtain compound 10-19; compound 10-19 undergoes amino group deprotection under an acidic condition to obtain compound 10-20, and in some cases, compound 10-20 is subjected to chiral resolution to obtain optical isomers 10-A and 10-B.

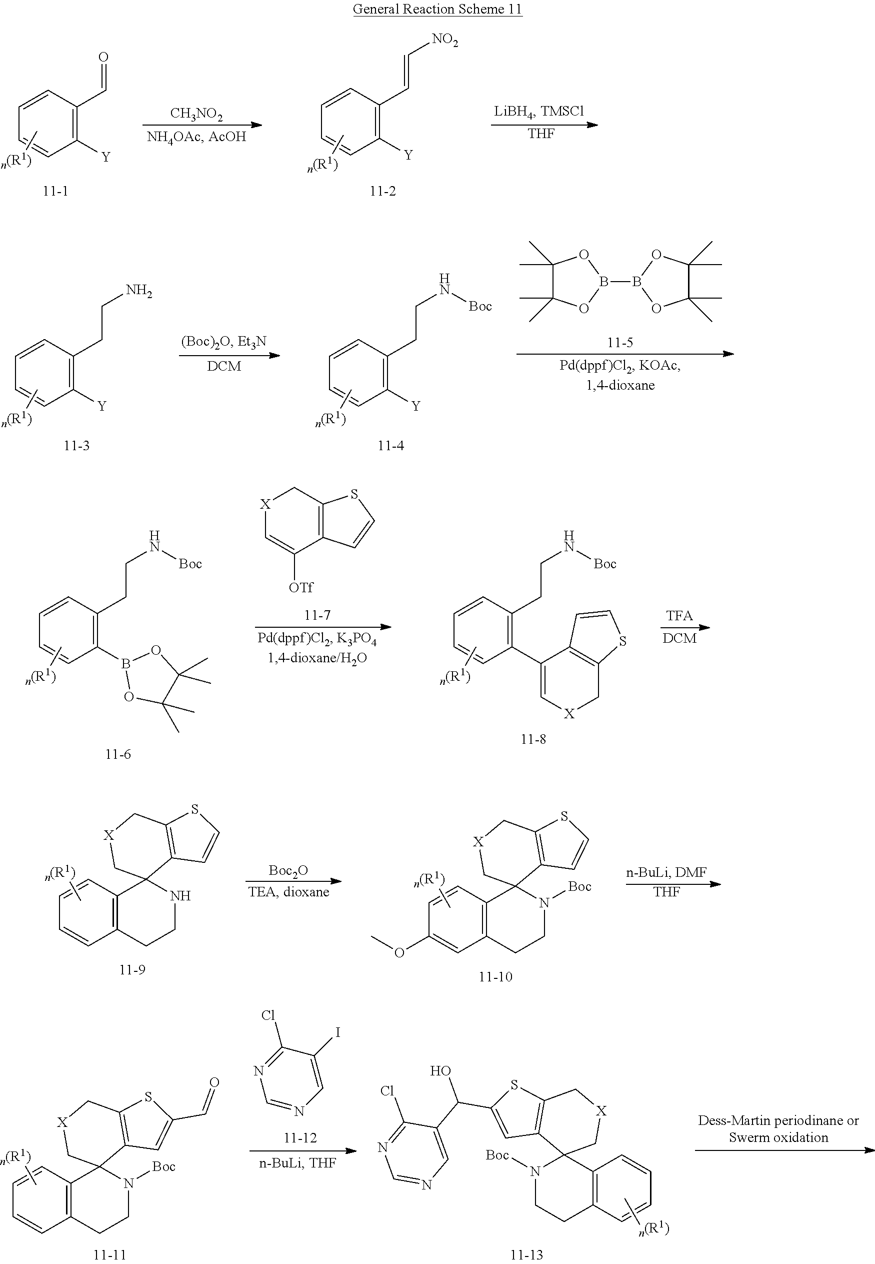

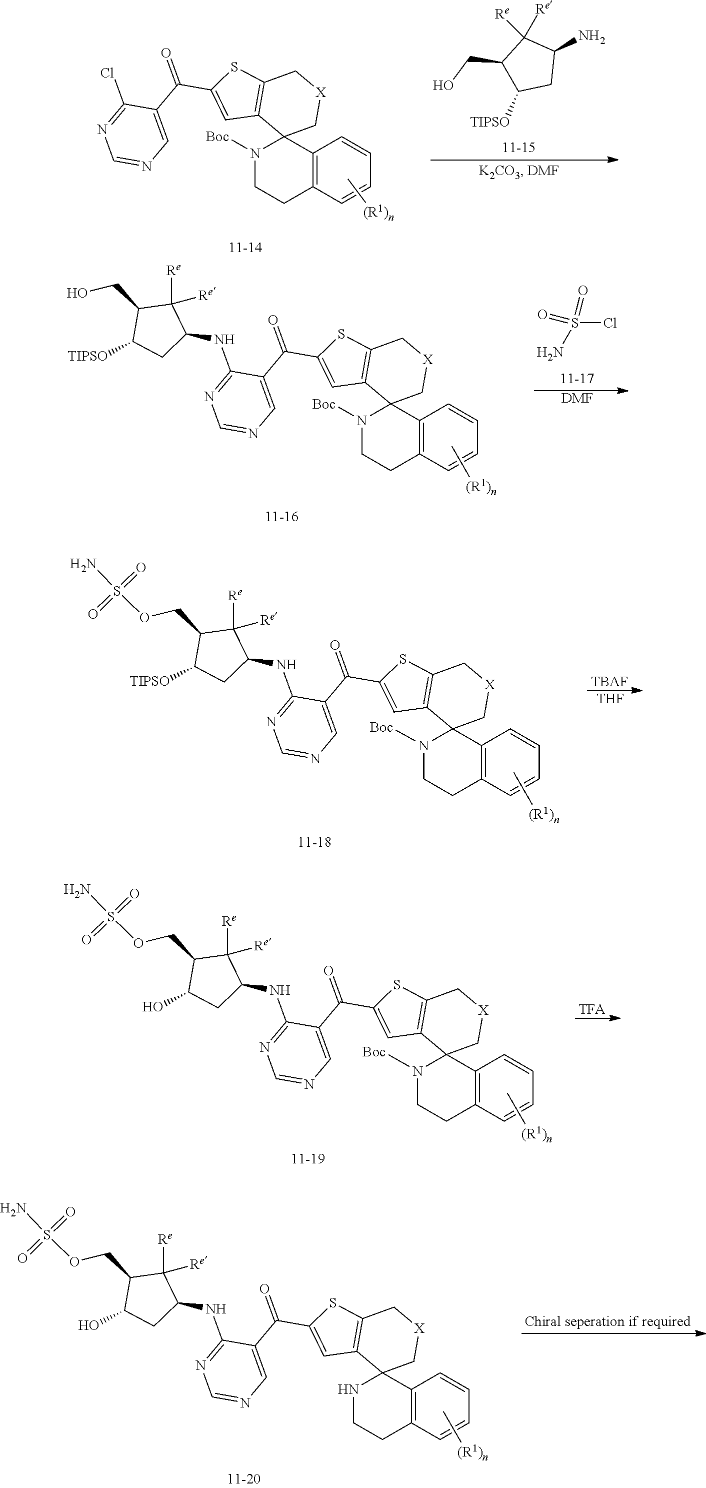

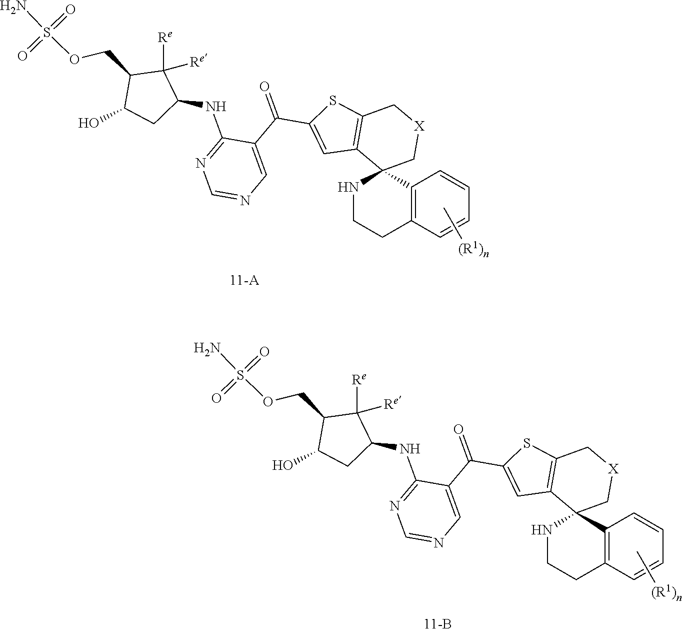

Embodiments of the compound of general formula (1) may be prepared according to general reaction scheme 11, wherein R¹, R^{e}, R^{e′}, and n are as defined above, Y represents Br, I, or OTf, X represents 0 or S, H represents hydrogen, N represents nitrogen, O represents oxygen, and Cl represents chlorine. As shown in general reaction scheme 11, compound 11-1 undergoes a condensation reaction with nitromethane to produce compound 11-2, and compound 11-2 is reduced to obtain amine 11-3; compound 11-3 is protected by (Boc)₂O to obtain compound 11-4; compound 11-4 undergoes a coupling reaction with compound 11-5 under the catalysis of Pd to obtain compound 11-6; compound 11-6 undergoes a coupling reaction with compound 11-7 under the catalysis of Pd to obtain compound 11-8; compound 11-8 undergoes a cyclization reaction under the condition of trifluoroacetic acid and dichloromethane to produce compound 11-9, and compound 11-9 is protected by (Boc)₂O to obtain compound 11-10; compound 11-10 reacts with DMF under the condition of n-butyllithium to obtain aldehyde 11-11, and aldehyde 11-11 undergoes an addition reaction with compound 11-12 under the condition of n-butyllithium to obtain compound 11-13; compound 11-13 is subjected to Dess-Martin oxidation or Swern oxidation to obtain ketone 11-14, and ketone 11-14 undergoes a substitution reaction with compound 11-15 under an alkaline condition to produce compound 11-16; compound 11-16 reacts with compound 11-17 to produce compound 11-18, and compound 11-18 undergoes deprotection by TBAF to obtain compound 11-19; compound 11-19 undergoes amino group deprotection under an acidic condition to obtain compound 11-20, and in some cases, compound 11-20 is subjected to chiral resolution to obtain optical isomers 11-A and 11-B.

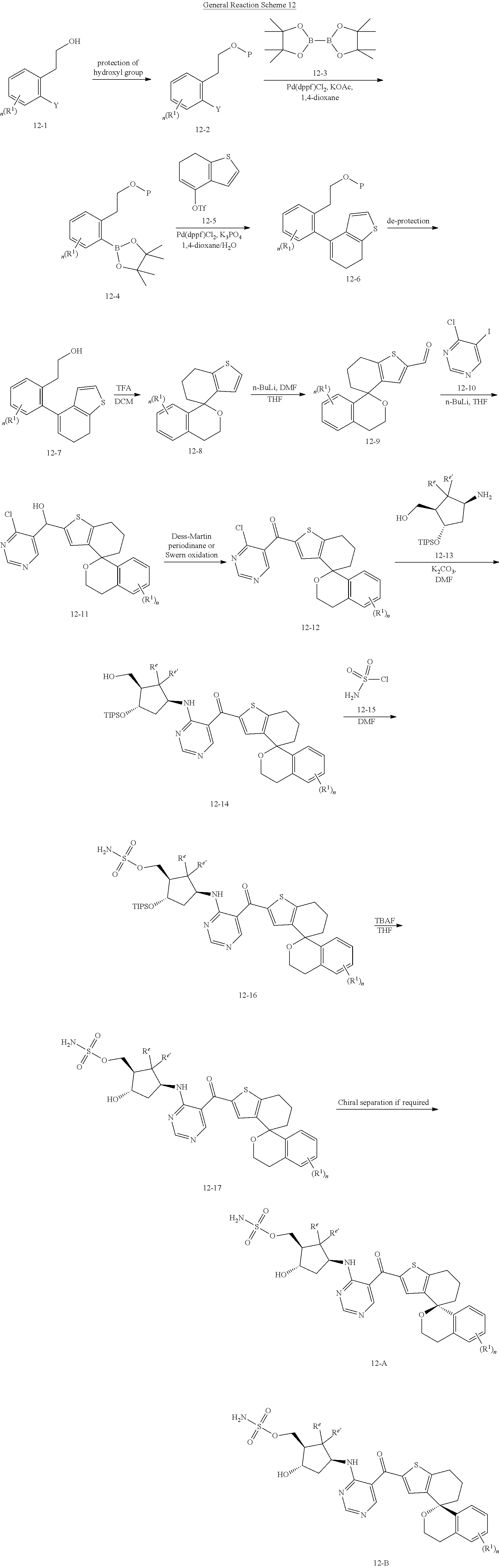

Embodiments of the compound of general formula (1) may be prepared according to general reaction scheme 12, wherein R¹, R^{e}, R^{e′}, and n are as defined above, Y represents Br, I, or OTf, H represents hydrogen, P represents a hydroxyl-protecting group, H represents hydrogen, N represents nitrogen, O represents oxygen, and Cl represents chlorine. As shown in general reaction scheme 12, compound 12-1 is protected to obtain compound 12-2; compound 12-2 undergoes a coupling reaction with compound 12-3 under the catalysis of Pd to obtain compound 12-4; compound 12-4 undergoes a coupling reaction with compound 12-5 under the catalysis of Pd to obtain compound 12-6; compound 12-6 is deprotected to obtain compound 12-7; compound 12-7 undergoes a cyclization reaction under the condition of trifluoroacetic acid and dichloromethane to produce compound 12-8; compound 12-8 reacts with DMF under the condition of n-butyllithium to obtain aldehyde 12-9; aldehyde 12-9 undergoes an addition reaction with compound 12-10 under the condition of n-butyllithium to obtain compound 12-11; compound 12-11 is subjected to Dess-Martin oxidation or Swern oxidation to obtain ketone 12-12; ketone 12-12 undergoes a substitution reaction with compound 12-13 under an alkaline condition to produce compound 12-14; compound 12-14 reacts with compound 12-15 to produce compound 12-16; compound 12-16 is deprotected by TBAF to obtain compound 12-17; and in some cases, compound 12-17 is subjected to chiral resolution to obtain optical isomers 12-A and 12-B.

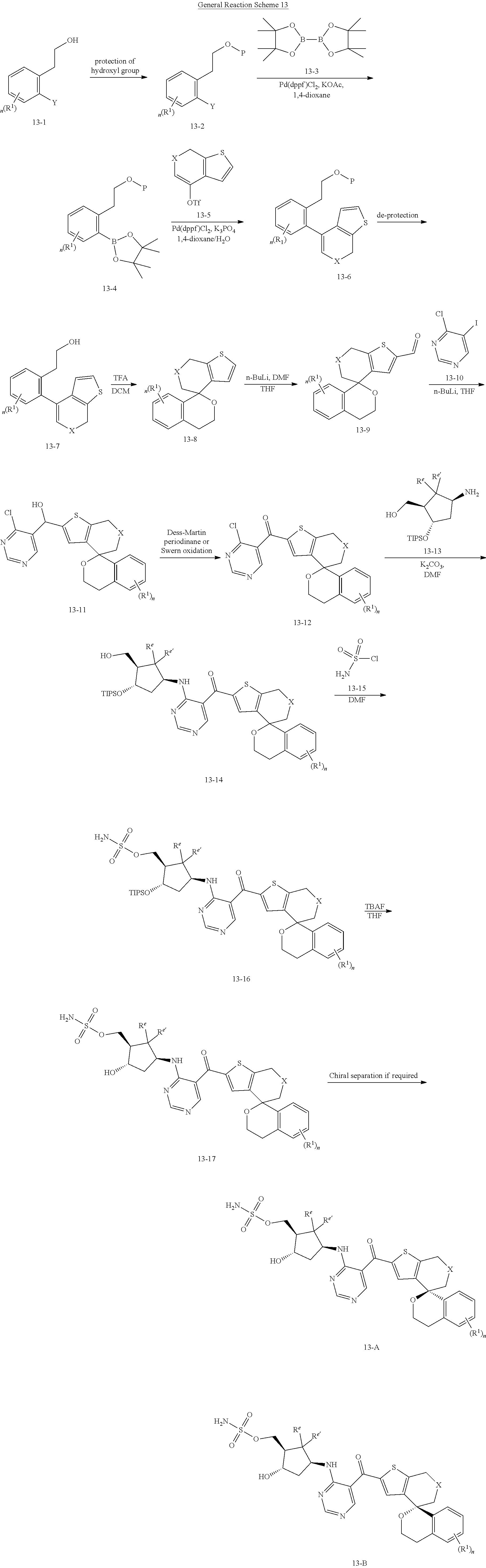

Embodiments of the compound of general formula (1) may be prepared according to general reaction scheme 13, wherein R¹, R^{e}, R^{e′}, and n are as defined above, Y represents Br, I, or OTf, H represents hydrogen, X represents O or S, P represents a hydroxyl-protecting group, H represents hydrogen, N represents nitrogen, O represents oxygen, and Cl represents chlorine. As shown in general reaction scheme 13, compound 13-1 is protected to obtain compound 13-2; compound 13-2 undergoes a coupling reaction with compound 13-3 under the catalysis of Pd to obtain compound 13-4; compound 13-4 undergoes a coupling reaction with compound 13-5 under the catalysis of Pd to obtain compound 13-6; compound 13-6 is deprotected to obtain compound 13-7; compound 13-7 undergoes a cyclization reaction under the condition of trifluoroacetic acid and dichloromethane to produce compound 13-8; compound 13-8 reacts with DMF under the condition of n-butyllithium to obtain aldehyde 13-9; aldehyde 13-9 undergoes an addition reaction with compound 13-10 under the condition of n-butyllithium to obtain compound 13-11; compound 13-11 is subjected to Dess-Martin oxidation or Swern oxidation to obtain ketone 13-12; ketone 13-12 undergoes a substitution reaction with compound 13-13 under an alkaline condition to produce compound 13-14; compound 13-14 reacts with compound 13-15 to produce compound 13-16; compound 13-16 is deprotected by TBAF to obtain compound 13-17; and in some cases, compound 13-17 is subjected to chiral resolution to obtain optical isomers 13-A and 13-B.

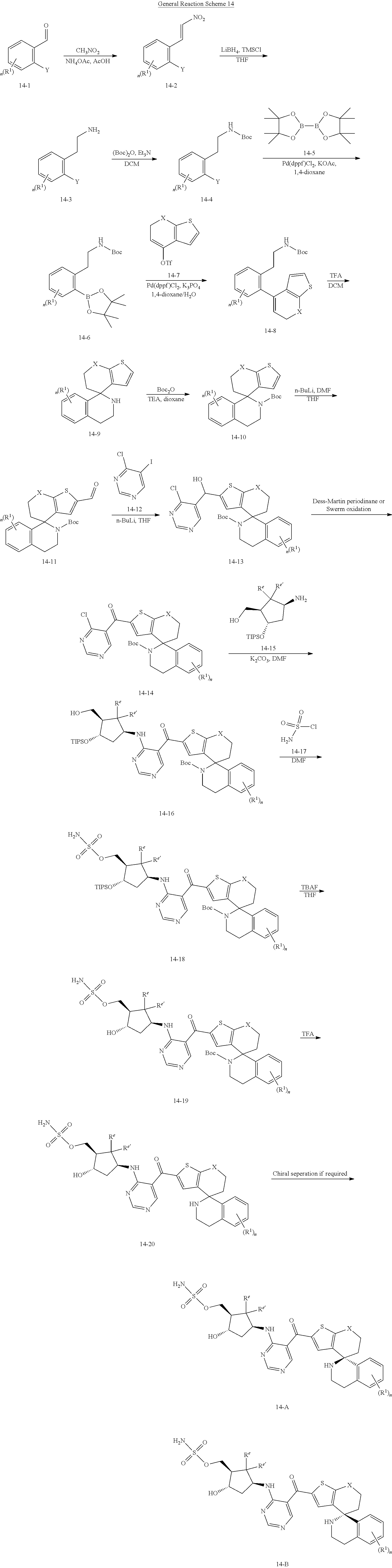

Embodiments of the compound of general formula (1) may be prepared according to general reaction scheme 14, wherein R¹, R^{e}, R^{e′}, and n are as defined above, Y represents Br, I, or OTf, X represents 0 or S, H represents hydrogen, N represents nitrogen, O represents oxygen, and Cl represents chlorine. As shown in general reaction scheme 14, compound 14-1 undergoes a condensation reaction with nitromethane to produce compound 14-2, and compound 14-2 is reduced to obtain amine 14-3; compound 14-3 is protected by (Boc)₂O to obtain compound 14-4; compound 14-4 undergoes a coupling reaction with compound 14-5 under the catalysis of Pd to obtain compound 14-6; compound 14-6 undergoes a coupling reaction with compound 14-7 under the catalysis of Pd to obtain compound 14-8; compound 14-8 undergoes a cyclization reaction under the condition of trifluoroacetic acid and dichloromethane to produce compound 14-9, and compound 14-9 is protected by (Boc)₂O to obtain compound 14-10; compound 14-10 reacts with DMF under the condition of n-butyllithium to obtain aldehyde 14-11, and aldehyde 14-11 undergoes an addition reaction with compound 14-12 under the condition of n-butyllithium to obtain compound 14-13; compound 14-13 is subjected to Dess-Martin oxidation or Swern oxidation to obtain ketone 14-14, and ketone 14-14 undergoes a substitution reaction with compound 14-15 under an alkaline condition to produce compound 14-16; compound 14-16 reacts with compound 14-17 to produce compound 14-18, and compound 14-18 undergoes deprotection by TBAF to obtain compound 14-19; compound 14-19 undergoes amino group deprotection under an acidic condition to obtain compound 14-20, and in some cases, compound 14-20 is subjected to chiral resolution to obtain optical isomers 14-A and 14-B.

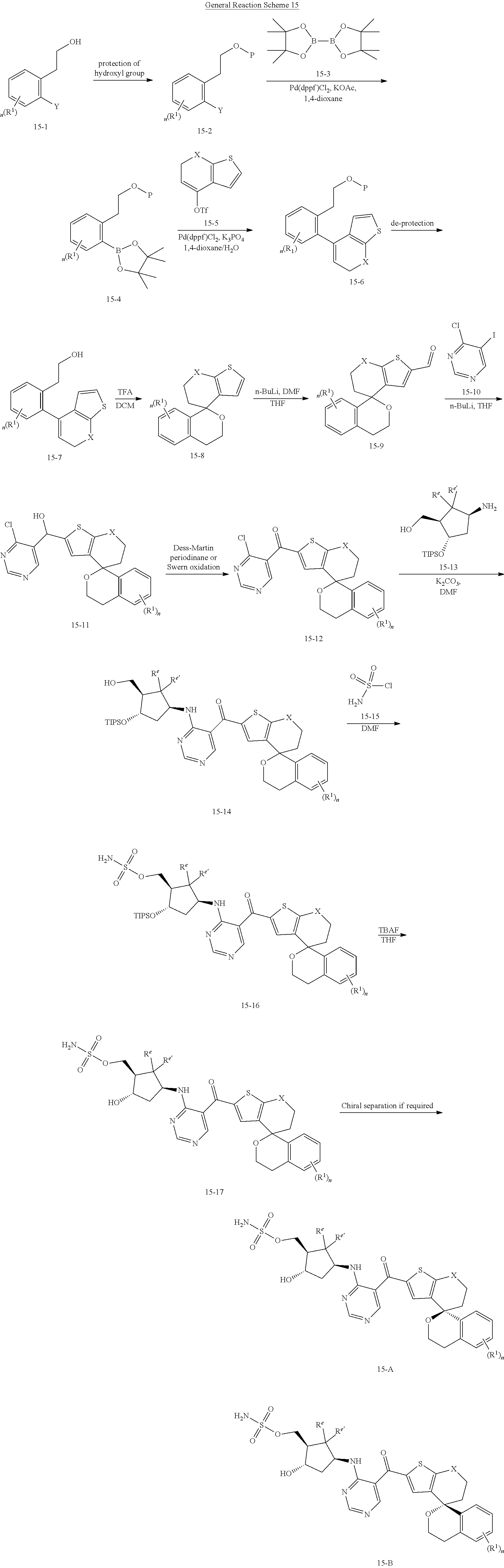

Embodiments of the compound of general formula (1) may be prepared according to general reaction scheme 15, wherein R¹, R^{e}, R^{e′}, and n are as defined above, Y represents Br, I, or OTf, H represents hydrogen, X represents O or S, P represents a hydroxyl-protecting group, H represents hydrogen, N represents nitrogen, O represents oxygen, and Cl represents chlorine. As shown in general reaction scheme 15, compound 15-1 is protected to obtain compound 15-2; compound 15-2 undergoes a coupling reaction with compound 15-3 under the catalysis of Pd to obtain compound 15-4; compound 15-4 undergoes a coupling reaction with compound 15-5 under the catalysis of Pd to obtain compound 15-6; compound 15-6 is deprotected to obtain compound 15-7; compound 15-7 undergoes a cyclization reaction under the condition of trifluoroacetic acid and dichloromethane to produce compound 15-8; compound 15-8 reacts with DMF under the condition of n-butyllithium to obtain aldehyde 15-9; aldehyde 15-9 undergoes an addition reaction with compound 15-10 under the condition of n-butyllithium to obtain compound 15-11; compound 15-11 is subjected to Dess-Martin oxidation or Swern oxidation to obtain ketone 15-12; ketone 15-12 undergoes a substitution reaction with compound 15-13 under an alkaline condition to produce compound 15-14; compound 15-14 reacts with compound 15-15 to produce compound 15-16; compound 15-16 is deprotected by TBAF to obtain compound 15-17; and in some cases, compound 15-17 is subjected to chiral resolution to obtain optical isomers 15-A and 15-B.

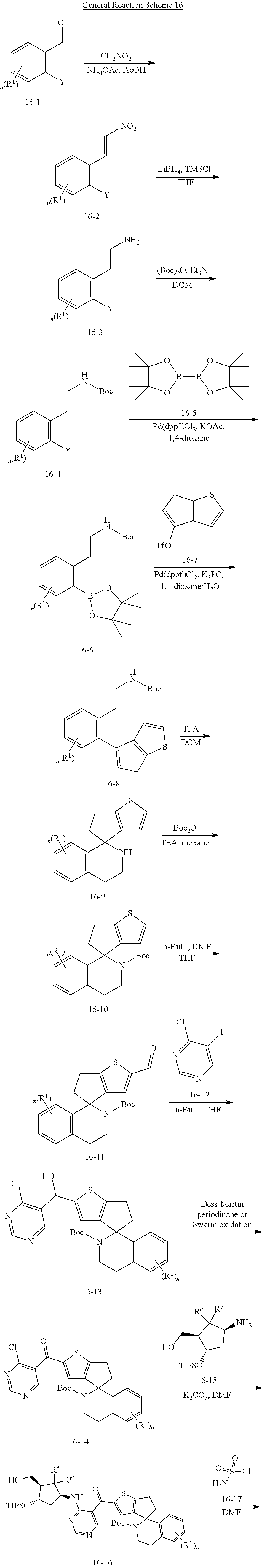

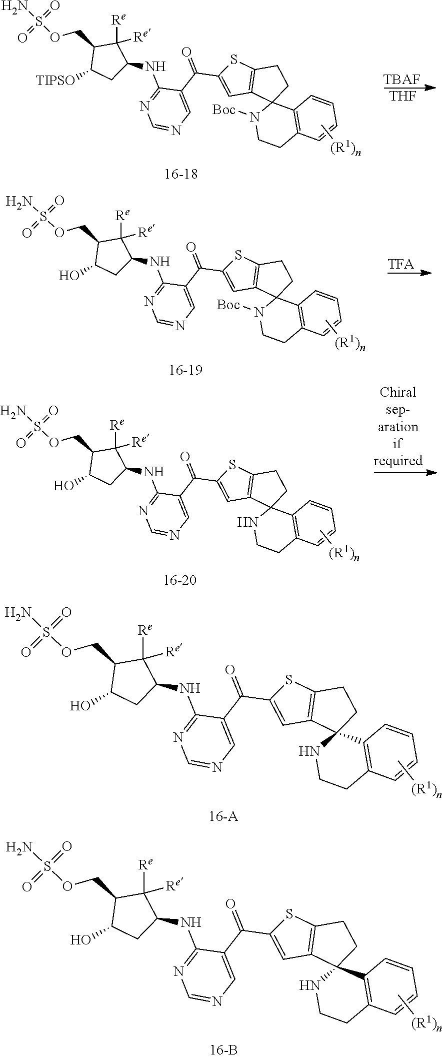

Embodiments of the compound of general formula (1) may be prepared according to general reaction scheme 16, wherein R¹, R^{e}, R^{e′}, and n are as defined above, Y represents Br, I, or OTf, H represents hydrogen, N represents nitrogen, O represents oxygen, and Cl represents chlorine. As shown in general reaction scheme 16, compound 16-1 undergoes a condensation reaction with nitromethane to produce compound 16-2, and compound 16-2 is reduced to obtain amine 16-3; compound 16-3 is protected by (Boc)₂O to obtain compound 16-4; compound 16-4 undergoes a coupling reaction with compound 16-5 under the catalysis of Pd to obtain compound 16-6; compound 16-6 undergoes a coupling reaction with compound 16-7 under the catalysis of Pd to obtain compound 16-8; compound 16-8 undergoes a cyclization reaction under the condition of trifluoroacetic acid and dichloromethane to produce compound 16-9, and compound 16-9 is protected by (Boc)₂O to obtain compound 16-10; compound 16-10 reacts with DMF under the condition of n-butyllithium to obtain aldehyde 16-11, and aldehyde 16-11 undergoes an addition reaction with compound 16-12 under the condition of n-butyllithium to obtain compound 16-13; compound 16-13 is subjected to Dess-Martin oxidation or Swern oxidation to obtain ketone 16-14, and ketone 16-14 undergoes a substitution reaction with compound 16-15 under an alkaline condition to produce compound 16-16; compound 16-16 reacts with compound 16-17 to produce compound 16-18, and compound 16-18 undergoes deprotection by TBAF to obtain compound 16-19; compound 16-19 undergoes amino group deprotection under an acidic condition to obtain compound 16-20, and in some cases, compound 16-20 is subjected to chiral resolution to obtain optical isomers 16-A and 16-B.

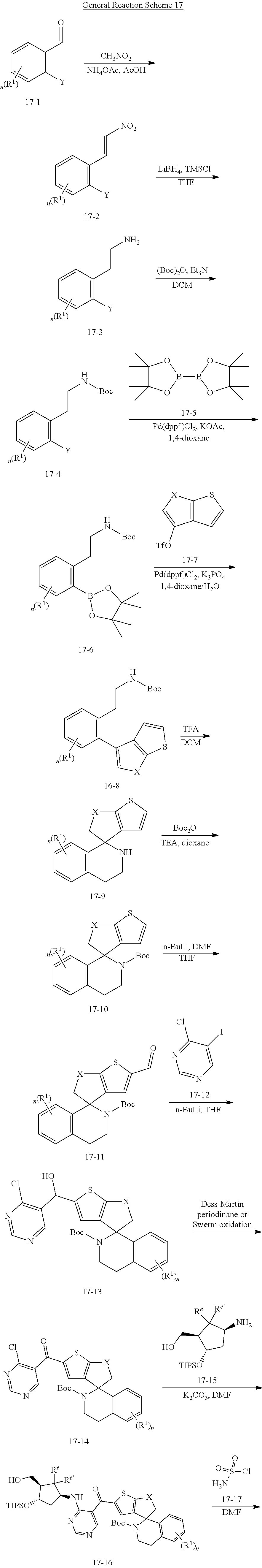

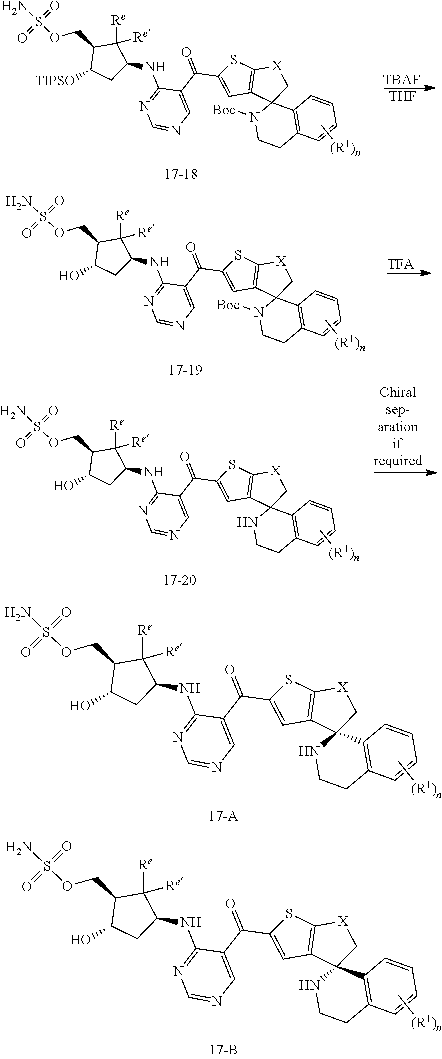

Embodiments of the compound of general formula (1) may be prepared according to general reaction scheme 17, wherein R¹, R^{e}, R^{e′}, and n are as defined above, Y represents Br, I, or OTf, X represents 0 or S, H represents hydrogen, N represents nitrogen, O represents oxygen, and Cl represents chlorine. As shown in general reaction scheme 17, compound 17-1 undergoes a condensation reaction with nitromethane to produce compound 17-2, and compound 17-2 is reduced to obtain amine 17-3; compound 17-3 is protected by (Boc)₂O to obtain compound 17-4; compound 17-4 undergoes a coupling reaction with compound 17-5 under the catalysis of Pd to obtain compound 17-6; compound 17-6 undergoes a coupling reaction with compound 17-7 under the catalysis of Pd to obtain compound 17-8; compound 17-8 undergoes a cyclization reaction under the condition of trifluoroacetic acid and dichloromethane to produce compound 17-9, and compound 17-9 is protected by (Boc)₂O to obtain compound 17-10; compound 17-10 reacts with DMF under the condition of n-butyllithium to obtain aldehyde 17-11, and aldehyde 17-11 undergoes an addition reaction with compound 17-12 under the condition of n-butyllithium to obtain compound 17-13; compound 17-13 is subjected to Dess-Martin oxidation or Swern oxidation to obtain ketone 17-14, and ketone 17-14 undergoes a substitution reaction with compound 17-15 under an alkaline condition to produce compound 17-16; compound 17-16 reacts with compound 17-17 to produce compound 17-18, and compound 17-18 undergoes deprotection by TBAF to obtain compound 17-19; compound 17-19 undergoes amino group deprotection under an acidic condition to obtain compound 17-20, and in some cases, compound 17-20 is subjected to chiral resolution to obtain optical isomers 17-A and 17-B.

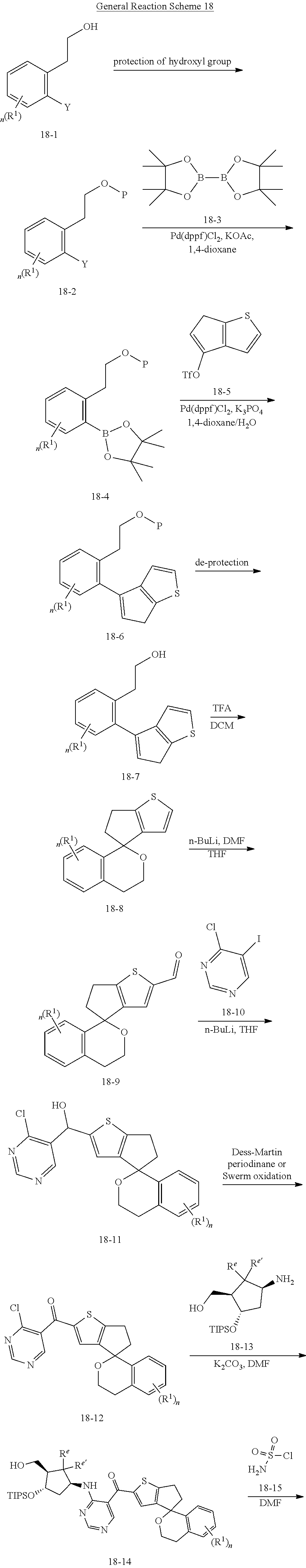

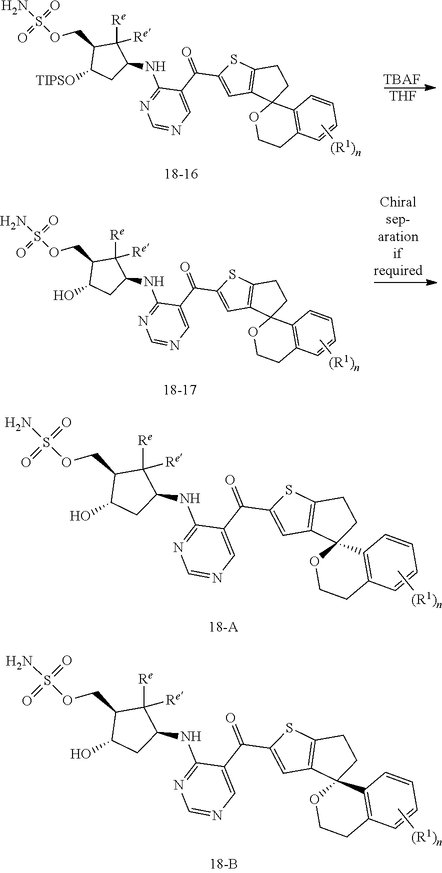

Embodiments of the compound of general formula (1) may be prepared according to general reaction scheme 18, wherein R¹, R^{e}, R^{e′}, and n are as defined above, Y represents Br, I, or OTf, H represents hydrogen, P represents a hydroxyl-protecting group, H represents hydrogen, N represents nitrogen, O represents oxygen, and Cl represents chlorine. As shown in general reaction scheme 18, compound 18-1 is protected to obtain compound 18-2; compound 18-2 undergoes a coupling reaction with compound 18-3 under the catalysis of Pd to obtain compound 18-4; compound 18-4 undergoes a coupling reaction with compound 18-5 under the catalysis of Pd to obtain compound 18-6; compound 18-6 is deprotected to obtain compound 18-7; compound 18-7 undergoes a cyclization reaction under the condition of trifluoroacetic acid and dichloromethane to produce compound 18-8; compound 18-8 reacts with DMF under the condition of n-butyllithium to obtain aldehyde 18-9; aldehyde 18-9 undergoes an addition reaction with compound 18-10 under the condition of n-butyllithium to obtain compound 18-11; compound 18-11 is subjected to Dess-Martin oxidation or Swern oxidation to obtain ketone 18-12; ketone 18-12 undergoes a substitution reaction with compound 18-13 under an alkaline condition to produce compound 18-14; compound 18-14 reacts with compound 18-15 to produce compound 18-16; compound 18-16 is deprotected by TBAF to obtain compound 18-17; and in some cases, compound 18-17 is subjected to chiral resolution to obtain optical isomers 18-A and 18-B.

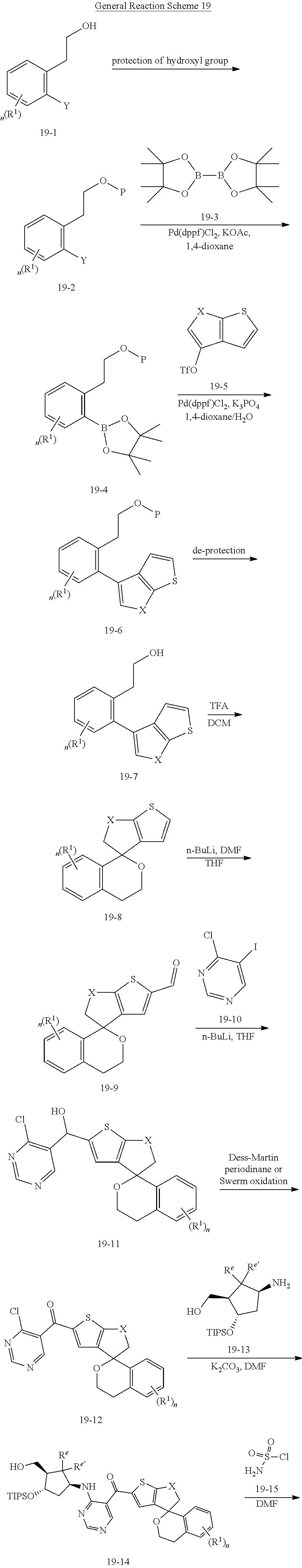

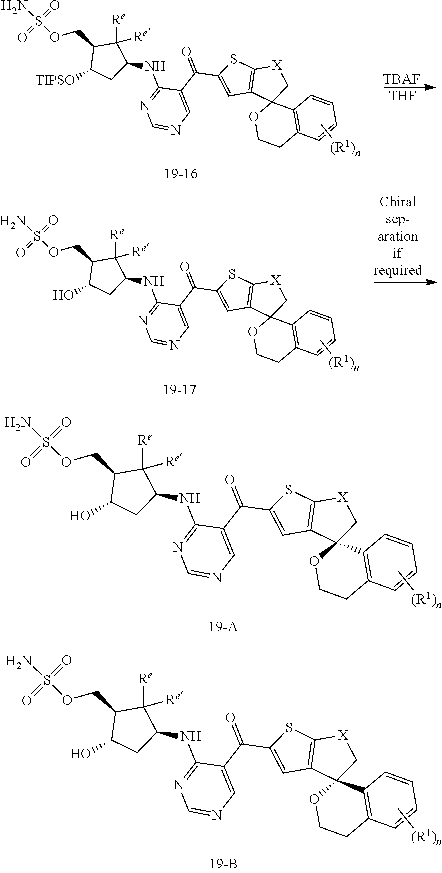

Embodiments of the compound of general formula (1) may be prepared according to general reaction scheme 19, wherein R¹, R^{e}, R^{e′}, and n are as defined above, Y represents Br, I, or OTf, H represents hydrogen, X represents O or S, P represents a hydroxyl-protecting group, H represents hydrogen, N represents nitrogen, O represents oxygen, and Cl represents chlorine. As shown in general reaction scheme 19, compound 19-1 is protected to obtain compound 19-2; compound 19-2 undergoes a coupling reaction with compound 19-3 under the catalysis of Pd to obtain compound 19-4; compound 19-4 undergoes a coupling reaction with compound 19-5 under the catalysis of Pd to obtain compound 19-6; compound 19-6 is deprotected to obtain compound 19-7; compound 19-7 undergoes a cyclization reaction under the condition of trifluoroacetic acid and dichloromethane to produce compound 19-8; compound 19-8 reacts with DMF under the condition of n-butyllithium to obtain aldehyde 19-9; aldehyde 19-9 undergoes an addition reaction with compound 19-10 under the condition of n-butyllithium to obtain compound 19-11; compound 19-11 is subjected to Dess-Martin oxidation or Swern oxidation to obtain ketone 19-12; ketone 19-12 undergoes a substitution reaction with compound 19-13 under an alkaline condition to produce compound 19-14; compound 19-14 reacts with compound 19-15 to produce compound 19-16; compound 19-16 is deprotected by TBAF to obtain compound 19-17; and in some cases, compound 19-17 is subjected to chiral resolution to obtain optical isomers 19-A and 19-B.

### Further Forms of Compounds

“Pharmaceutically acceptable” herein refers to a substance, such as a carrier or diluent, which will not lead to loss of biological activity or properties of a compound and is relatively non-toxic. For example, when an individual is given a substance, the substance will not cause undesired biological effects or interact with any component contained therein in a deleterious manner.

The term “pharmaceutically acceptable salt” refers to a form of a compound that does not cause significant irritation to the organism receiving the administration or eliminate the biological activity and properties of the compound. In certain specific aspects, the pharmaceutically acceptable salt is obtained by subjecting the compound of the general formula to a reaction with acids or bases, wherein the acids or bases include, but are not limited to, those found in Stahl and Wermuth, Handbook of Pharmaceutical Salts: Properties, Selection, and Use, 1^{st} Ed., (Wiley, 2002).

It should be understood that references to pharmaceutically acceptable salts include solvent addition forms or crystalline forms, especially solvates or polymorphs. A solvate contains either stoichiometric or non-stoichiometric amount of solvent and is selectively formed during crystallization in a pharmaceutically acceptable solvent such as water and ethanol. Hydrates are formed when the solvent is water, or alcoholates are formed when the solvent is ethanol. The solvates of the compound of the general formula are conveniently prepared or formed according to the methods described herein. For example, hydrates of the compound of the general formula are conveniently prepared by recrystallization in a mixed solvent of water/organic solvent, wherein the organic solvent used includes, but is not limited to, tetrahydrofuran, acetone, ethanol or methanol. Furthermore, the compounds described herein may be present in either a non-solvated form or a solvated form. In general, the solvated forms are considered equivalent to the non-solvated forms for purposes of the compounds and methods provided herein.

In other specific embodiments, the compound of the general formula is prepared in different forms including, but not limited to, amorphous, pulverized and nanoparticle forms. In addition, the compound of the general formula includes crystalline forms, and may also be polymorphs. Polymorphs include different lattice arrangements of the same elements of a compound. Polymorphs generally have different X-ray diffraction spectra, infrared spectra, melting points, density, hardness, crystalline forms, optical and electrical properties, stability and solubility. Different factors such as a recrystallization solvent, crystallization rate, and storage temperature may lead to a single dominant crystalline form.

In another aspect, the compound of the general formula may have a chiral center and/or axial chirality, and therefore may be present in the form of a racemate, a racemic mixture, a single enantiomer, a diastereomeric compound, a single diastereomer and a cis-trans isomer. Each chiral center or axial chirality will independently produce two optical isomers, and all possible optical isomers, diastereomeric mixtures, and pure or partially pure compounds are included within the scope of the present disclosure. The present disclosure is meant to include all such isomeric forms of these compounds.

The compound of the present disclosure may contain unnatural proportions of atomic isotopes at one or more of the atoms that constitute the compound. For example, the compound may be labeled with radioactive isotopes, such as tritium (³H), iodine-125 (¹²⁵I), and C-14 (¹⁴C). For another example, deuterium can be used to substitute a hydrogen atom to form a deuterated compound. The bond formed by deuterium and carbon is stronger than that formed by ordinary hydrogen and carbon. Compared with an undeuterated medicament, the deuterated medicament generally has the advantages of reduced toxic and side effects, increased pharmaceutical stability, enhanced efficacy, prolonged pharmaceutical in vivo half-life and the like. All isotopic variations of the compound of the present disclosure, whether radioactive or not, are contained within the scope of the present disclosure.

Any atom of the compound of the present disclosure, unless otherwise specified, refers to an isotope of the atom in stable state of the compound. Unless otherwise specified, when a site in a molecular structure is selected as “H” or “hydrogen”, the site should be understood as having the natural abundance of the hydrogen isotope. Similarly, unless otherwise specified, when a site is selected as “D” or “deuterium”, the site should be understood to have a deuterium isotopic abundance that is at least 3000 times its natural abundance (the natural abundance of the deuterium isotope is 0.015%).

More preferably, each deuterated site of the deuterated compounds of the present disclosure has a deuterium atom abundance that is at least 3500 times its natural abundance (52.2% deuterium atom enrichment). More preferably, the deuterium atom abundance is at least 4500 times the natural abundance (67.5% deuterium atom enrichment). More preferably, the deuterium atom abundance is at least 5000 times the natural abundance (75% deuterium atom enrichment). More preferably, the deuterium atom abundance is at least 6000 times the natural abundance (90% deuterium atom enrichment). More preferably, the deuterium atom abundance is at least 6333 times the natural abundance (95% deuterium atom enrichment). More preferably, the deuterium atom abundance is at least 6466.7 times the natural abundance (97% deuterium atom enrichment). More preferably, the deuterium atom abundance is at least 6600 times the natural abundance (99% deuterium atom enrichment). More preferably, the deuterium atom abundance is at least 6633.3 times the natural abundance (99.5% deuterium atom enrichment).

### Terminology

Unless otherwise stated, the terms used in the present application, including those in the specification and claims, are defined as follows. It must be noted that in the specification and the appended claims, the singular forms “a” and “an” include plural meanings unless clearly indicated otherwise. Unless otherwise stated, conventional methods for mass spectrometry, nuclear magnetic resonance spectroscopy, HPLC, protein chemistry, biochemistry, recombinant DNA techniques and pharmacology are used. As used herein, “or” or “and” refers to “and/or” unless otherwise stated.

Unless otherwise specified, “alkyl” refers to a saturated aliphatic hydrocarbon group, including linear and branched groups containing 1 to 6 carbon atoms. Lower alkyl groups containing 1 to 4 carbon atoms, such as methyl, ethyl, propyl, 2-propyl, n-butyl, isobutyl, or tert-butyl, are preferred. Lower alkyl groups containing 1-3 carbon atoms, such as methyl, ethyl, propyl, and 2-propyl, are further preferred. As used herein, “alkyl” includes unsubstituted and substituted alkyl, particularly alkyl substituted with one or more halogens. Preferred alkyl is selected from CH₃, CH₃CH₂, CF₃, CHF₂, CF₃CH₂, CF₃(CH₃)CH, ⁱPr, ⁿPr, ⁱBu, ⁿBu, and ^{t}Bu.

Unless otherwise specified, “alkylene” refers to a divalent alkyl as defined above. Examples of alkylene include, but are not limited to, methylene and ethylene.

Unless otherwise specified, “alkenyl” refers to an unsaturated aliphatic hydrocarbon group containing carbon-carbon double bonds, including linear or branched groups containing 1 to 14 carbon atoms. Lower alkenyl groups containing 1 to 4 carbon atoms, such as vinyl, 1-propenyl, 1-butenyl, or 2-methylpropenyl, are preferred. Lower alkenyl groups containing 1 to 2 carbon atoms are further preferred.

Unless otherwise specified, “alkenylene” refers to a divalent alkenyl as defined above.

Unless otherwise specified, “alkynyl” refers to an unsaturated aliphatic hydrocarbon group containing carbon-carbon triple bonds, including linear and branched groups containing 1 to 14 carbon atoms. Lower alkynyl groups containing 1 to 4 carbon atoms, such as ethynyl, 1-propynyl, or 1-butynyl, are preferred. Lower alkynyl groups containing 1 to 2 carbon atoms are further preferred.

Unless otherwise specified, “alkynylene” refers to a divalent alkynyl as defined above.

Unless otherwise specified, “cycloalkyl” refers to a non-aromatic hydrocarbon ring system (monocyclic, bicyclic, or polycyclic), and is preferably a non-aromatic hydrocarbon ring system containing 3 to 14 ring carbon atoms (C₃₋₁₄ cycloalkyl). In some embodiments, cycloalkyl has 3 to 10 ring carbon atoms (C₃₋₁₀ cycloalkyl). In some embodiments, cycloalkyl has 3 to 8 ring carbon atoms (C₃₋₈ cycloalkyl). In some embodiments, cycloalkyl has 3 to 7 ring carbon atoms (C₃₋₇ cycloalkyl). In some embodiments, cycloalkyl has 3 to 6 ring carbon atoms (C₃₋₆ cycloalkyl). In some embodiments, cycloalkyl has 4 to 6 ring carbon atoms (C₄₋₆ cycloalkyl). In some embodiments, cycloalkyl has 5 to 6 ring carbon atoms (C₅₋₆ cycloalkyl). In some embodiments, cycloalkyl has 5 to 10 ring carbon atoms (C₅₋₁₀ cycloalkyl). For cycloalkyl, partially unsaturated cycloalkyl may be referred to as “cycloalkenyl” if the carbocyclic ring contains at least one double bond, or “cycloalkynyl” if the carbocyclic ring contains at least one triple bond. Cycloalkyl may include monocyclic or polycyclic groups (e.g., having 2, 3, or 4 fused rings) and spiro rings. In some embodiments, cycloalkyl is monocyclic. In some embodiments, cycloalkyl is bicyclic. In some embodiments, cycloalkyl is monocyclic or bicyclic. In some embodiments, cycloalkyl is tricyclic. The ring carbon atoms of cycloalkyl may optionally be oxidized to form an oxo or thio group. Cycloalkyl further includes cycloalkylene. In some embodiments, cycloalkyl contains 0, 1, or 2 double bonds. In some embodiments, cycloalkyl contains 1 or 2 double bonds (partially unsaturated cycloalkyl). In some embodiments, cycloalkyl may be fused to aryl, heteroaryl, cycloalkyl, and heterocycloalkyl. In some embodiments, cycloalkyl may be fused to aryl, cycloalkyl and heterocycloalkyl. In some embodiments, cycloalkyl may be fused to aryl and heterocycloalkyl. In some embodiments, cycloalkyl may be fused to aryl and cycloalkyl. Examples of cycloalkyl include cyclopropyl, cyclobutyl, cyclopentyl, cyclohexyl, cycloheptyl, cyclopentenyl, cyclohexenyl, cyclohexadienyl, cycloheptatrienyl, norcamphanyl, norpinanyl, norcamyl, bicyclo[1.1.1]pentyl, bicyclo[2.1.1]hexyl and the like.

Unless otherwise specified, “cycloalkylene” refers to a divalent cycloalkyl as defined above.

Unless otherwise specified, “alkoxy” refers to an alkyl group that bonds to the rest of the molecule through an ether oxygen atom. Representative alkoxy groups are those having 1-6 carbon atoms, such as methoxy, ethoxy, propoxy, isopropoxy, butoxy, isobutoxy, sec-butoxy, and tert-butoxy. As used herein, “alkoxy” includes unsubstituted and substituted alkoxy, particularly alkoxy substituted with one or more halogens. Preferred alkoxy is selected from OCH₃, OCF₃, CHF₂O, CF₃CH₂O, ⁱ-PrO, ⁿ-PrO, ⁱ-BuO, ⁿ-BuO, and ^{t}BuO.

Unless otherwise specified, “aryl” refers to an aromatic hydrocarbon group, which is monocyclic or polycyclic; for example, a monocyclic aryl ring is fused to one or more carbocyclic aromatic groups. Examples of aryl include, but are not limited to, phenyl, naphthyl, and phenanthryl.

Unless otherwise specified, “aryloxy” refers to an aryl group that bonds to the rest of the molecule through an ether oxygen atom. Examples of aryloxy include, but are not limited to, phenoxy and naphthoxy.

Unless otherwise specified, “arylene” refers to a divalent aryl as defined above. Examples of arylene include, but are not limited to, phenylene, naphthylene, and phenanthrylene.

Unless otherwise specified, “heteroaryl” refers to an aromatic group containing substitution of one or more heteroatoms or an unsubstituted aromatic group, preferably a 5- to 14-membered aromatic group containing 1 to 4 heteroatoms selected from oxygen, sulfur, and nitrogen, and more preferably a 5- to 9-membered aromatic group containing 1 to 2 heteroatoms optionally selected from oxygen, sulfur, or nitrogen. The heteroatoms are independently selected from O, N, or S, and the number of the heteroatoms is preferably 1, 2, or 3. Heteroaryl is monocyclic or polycyclic. Monocyclic heteroaryl is preferably a 5- to 6-membered aromatic group containing 1 to 3 heteroatoms optionally selected from oxygen, nitrogen, and sulfur. More preferably, monocyclic heteroaryl is a 5- to 6-membered aromatic group containing 1 to 2 heteroatoms optionally selected from oxygen, nitrogen, and sulfur. More preferably, monocyclic heteroaryl is a 5- to 6-membered aromatic group containing 1 heteroatom optionally selected from oxygen, nitrogen, and sulfur. In some embodiments, a monocyclic heteroaryl ring is fused to one or more carbocyclic aromatic groups or other monocyclic heterocycloalkyl groups. Examples of heteroaryl include, but are not limited to, pyridinyl, pyridazinyl, imidazolyl, pyrimidinyl, pyrazolyl, triazolyl, pyrazinyl, quinolinyl, isoquinolinyl, furanyl, thienyl, isoxazolyl, thiazolyl, oxazolyl, 1,2,4-oxadiazolyl, 1,3,4-oxadiazolyl, 1,2,5-oxadiazolyl, isothiazolyl, pyrrolyl, indolyl, benzimidazolyl, benzofuranyl, benzothiazolyl, benzothienyl, benzoxazolyl, benzopyridinyl, pyrrolopyrimidinyl, 1H-pyrrolo[3,2-b]pyridinyl, 1H-pyrrolo[2,3-c]pyridinyl, 1H-pyrrolo[3,2-c]pyridinyl, 1H-pyrrolo[2,3-b]pyridinyl,

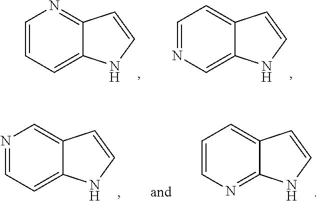

Unless otherwise specified, “heteroarylene” refers to a divalent heteroaryl as defined above.

Unless otherwise specified, “heterocycloalkyl” refers to a non-aromatic ring or ring system, which may optionally contain one or more alkenylene as part of the ring structure, having at least one heteroatom ring member independently selected from boron, phosphorus, nitrogen, sulfur, oxygen, and selenium; heterocycloalkyl is preferably a saturated or partially unsaturated ring containing 1 to 4 heteroatoms selected from oxygen, sulfur, or nitrogen, more preferably a saturated or partially unsaturated ring containing 1 to 2 heteroatoms selected from oxygen, sulfur, or nitrogen. In some embodiments, heterocycloalkyl is a 5- to 8-membered non-aromatic ring containing ring carbon atoms and 1 to 4 ring heteroatoms, and each heteroatom is independently and optionally selected from nitrogen, oxygen, and sulfur (5- to 8-membered heterocycloalkyl). Heterocycloalkyl is a 5- to 6-membered non-aromatic ring containing ring carbon atoms and 1 to 4 ring heteroatoms, and each heteroatom is independently and optionally selected from nitrogen, oxygen, and sulfur (5- to 6-membered heterocycloalkyl). In some embodiments, 5- to 6-membered heterocycloalkyl contains 1 to 3 ring heteroatoms independently selected from nitrogen, oxygen, and sulfur. In some embodiments, 5- to 6-membered heterocycloalkyl contains 1 to 2 ring heteroatoms independently selected from nitrogen, oxygen, and sulfur. In some embodiments, 5- to 6-membered heterocycloalkyl contains 1 ring heteroatom independently selected from nitrogen, oxygen, and sulfur. Partially unsaturated heterocycloalkyl may be referred to as “heterocycloalkenyl” if heterocycloalkyl contains at least one double bond, or “heterocycloalkynyl” if the heterocycloalkyl contains at least one triple bond. Heterocycloalkyl may include monocyclic, bicyclic, spiro ring, or polycyclic (e.g., having two fused or bridged rings) systems. In some embodiments, heterocycloalkyl is a monocyclic group having 1, 2, or 3 heteroatoms independently selected from nitrogen, sulfur, and oxygen. The ring carbon atoms and heteroatoms of heterocycloalkyl may optionally be oxidized to form oxo or thio groups or other oxidized bonds (e.g., C(O), S(O), C(S) or S(O)₂, and N-oxides), or the nitrogen atoms may be quaternized. Heterocycloalkyl may be attached via a ring carbon atom or a ring heteroatom. In some embodiments, heterocycloalkyl contains 0 to 3 double bonds. In some embodiments, heterocycloalkyl contains 0 to 2 double bonds. The definition of heterocycloalkyl further includes moieties (also referred to as partially unsaturated heterocyclic rings) having one or more aromatic rings fused to (i.e., sharing a bond with) the heterocycloalkyl ring, for example, benzo-derivatives of piperidine, morpholine, azepin, and thienyl. Heterocycloalkyl containing a fused aromatic ring may be attached via any ring atom, including ring atoms of the fused aromatic ring. Examples of heterocycloalkyl include, but are not limited to, azetidinyl, azepinyl, dihydrobenzofuranyl, dihydrofuranyl, dihydropyranyl, N-morpholinyl, 3-oxa-9-azaspiro[5.5]undecyl, 1-oxa-8-azaspiro[4.5]decyl, piperidinyl, piperazinyl, oxopiperazinyl, pyranyl, pyrrolidinyl, quininyl, tetrahydrofuranyl, tetrahydropyranyl, 1,2,3,4-tetrahydroquinolinyl, tropanyl, 4,5,6,7-tetrahydrothiazolo[5,4-c]pyridinyl, 4,5,6,7-tetrahydro-1H-imidazo[4,5-c]pyridine, N-methylpiperidinyl, tetrahydroimidazolyl, pyrazolidinyl, butyrolactam, valerolactam, imidazolidinonyl, hydantoinyl, dioxolanyl, phthalimidyl, pyrimidine-2,4(1H,3H)-dione, 1,4-dioxanyl, morpholinyl, thiomorpholinyl, thiomorpholinyl-S-oxide, thiomorpholinyl-S,S-oxide, piperazinyl, pyranyl, pyridonyl, 3-pyrrolinyl, thiopyranyl, pyronyl, tetrahydrothienyl, 2-azaspiro[3.3]heptanyl, indolinyl,

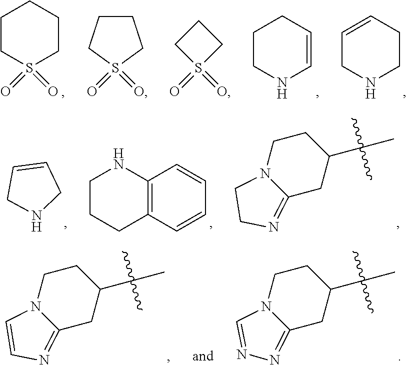

Unless otherwise specified, “heterocycloalkylene” refers to divalent heterocycloalkyl as defined above.

Unless otherwise specified, “oxo” refers to ═O; for example, a group formed by substitution of carbon with one oxo is “carbonyl

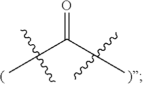 [SMILES: *C(*)=O]

a group formed by substitution of sulfur with one oxo is “sulfinyl

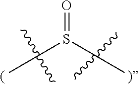 [SMILES: *S(*)=O]

and a group formed by substitution of sulfur with two oxo is “sulfonyl

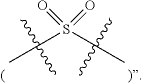

Unless otherwise specified, “halogen” (or halo) refers to fluorine, chlorine, bromine, or iodine. The term “halo” (or “halogenated”) before a group name indicates that the group is partially or fully halogenated, that is, substituted in any combination with F, Cl, Br, or I, preferably with F or Cl.

Unless otherwise specified, the term “substituted” means that one or more hydrogen atoms on a designated atom or group are substituted with one or more substituents other than hydrogen without exceeding the normal valence of the designated atom. For example, one or more hydrogens of alkyl, alkylene, alkenyl, alkynyl, hydroxy, amino, or the like may be substituted with one or more substituents. The substituents include, but are not limited to, alkyl, alkenyl, alkynyl, alkoxy, acyl, amino, amido, amidino, aryl, azido, carbamoyl, carboxy, carboxylate, cyano, guanidino, halogen, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof.

The definition of “substituted” does not include analogous indeterminate structures obtained by defining substituents having further substituents attached to infinity (e.g., substituted aryl having substituted alkyl is itself substituted with substituted aryl, which is further substituted with substituted heteroalkyl, and the like).

Unless otherwise specified, the maximum number of consecutive substitutions in the compound described herein is three. For example, the consecutive substitutions of substituted aryl with two other substituted aryls are limited to ((substituted aryl)substituted aryl)substituted aryl. Similarly, the definitions described above do not include impermissible substitution patterns (e.g., methyl substituted with 5 fluorines or heteroaryl having two adjacent oxygen ring atoms). Such impermissible substitution patterns are well known to those skilled in the art. When used to modify a chemical group, “substituted” may describe other chemical groups as defined herein. For example, the term “substituted aryl” includes, but is not limited to, “alkylaryl”. Unless otherwise specified, if a group is described as optionally substituted, any substituent of the group is itself unsubstituted. “Optional” or “optionally” means that the subsequently described event or circumstance may, but does not necessarily, occur, and the description includes instances where the event or circumstance occurs and instances where it does not.

Unless otherwise specified, it will be understood that the word “comprise” or variations thereof such as “comprises” or “comprising” refers to the inclusion of a stated element or integer or a group of elements or integers, but not the exclusion of any other element or integer or a group of elements or integers.

The substituent “—O—CH₂—O—” means that two oxygen atoms in the substituent are linked to two adjacent carbon atoms in the heterocycloalkyl, aryl, or heteroaryl, for example

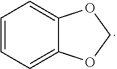

When the number of a linker group is 0, such as —(CH₂)₀—, it means that the linker group is a single bond.

When one of the variables is selected from a chemical bond, it means that the two groups linked by this variable are linked directly. For example, when L in X-L-Y represents a chemical bond, it means that the structure is actually X-Y.

The term “membered ring” includes any cyclic structure. The term “membered” is intended to refer to the number of backbone atoms that form a ring. For example, cyclohexyl, pyridinyl, pyranyl and thiopyranyl are six-membered rings, and cyclopentyl, pyrrolyl, furanyl, and thienyl are five-membered rings.

The term “moiety” refers to a specific portion or functional group of a molecule. A chemical moiety is generally considered to be a chemical entity contained in or attached to a molecule.

The term “isomer” refers to any tautomer, stereoisomer, atropisomer, isotopic isomer, enantiomer, or diastereomer of any compound of the present disclosure. The compound of the present disclosure may have one or more chiral centers or double bonds, and thus exists in the form of stereoisomers, e.g., double bond isomers (i.e., E/Z geometric isomers), or diastereomers (e.g., enantiomers (i.e., (+) or (−)) or cis/trans isomers).

The compound of the present disclosure therefore encompasses all corresponding stereoisomers, i.e., stereoisomerically pure (e.g., geometrically pure, enantiomerically pure, or diastereomerically pure) forms, as well as mixtures of enantiomers and stereoisomers, e.g., racemates. The mixtures of enantiomers and stereoisomers of the compound of the present disclosure may be resolved into their component enantiomers or stereoisomers by well-known methods, such as chiral gas chromatography and chiral high-performance liquid chromatography, and by crystallization of the compound in the form of chiral salt complexes or in chiral solvents. Enantiomers and stereoisomers may also be obtained from stereomerically pure or enantiomerically pure intermediates, reagents, and catalysts by well-known asymmetric synthetic methods.

The term “isotopic isomer” refers to distinct molecules that differ in structure only by their isotopic composition and are identical in the remaining structure.

The term “atropisomer” refers to a conformational stereoisomer that results when rotation about a single bond within a molecule is hindered or greatly slowed due to the steric interaction with other parts of the molecule, and the substituents at both ends of the single bond are asymmetric, i.e., the atropisomer does not require a stereocenter. In the case of sufficiently high rotational hindrance around the single bond and sufficiently slow interconversion between conformations, the separation of individual isomers may be allowed (LaPlante et al., J. Med. Chem. 2011, 54, 20, 7005), preferably by a chiral resolution method.

Unless otherwise stated the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond ( [symbol] ) or a straight dashed bond ( [symbol] ).

Unless otherwise stated, a single bond or a double bond is represented by [symbol] .

### Specific Pharmaceutical and Medical Terminology

The term “acceptable”, as used herein, means that a formula component or an active ingredient does not unduly and adversely affect a general therapeutic target's health.

The terms “treatment”, “treatment course”, and “therapy”, as used herein, include alleviating, inhibiting, or ameliorating a symptom or condition of a disease; inhibiting the development of complications; ameliorating or preventing underlying metabolic syndrome; inhibiting the development of a disease or symptom, e.g., controlling the progression of a disease or condition; alleviating a disease or symptom; leading to disease or symptom regression; and alleviating a complication caused by a disease or symptom, or preventing or treating a sign caused by a disease or symptom. As used herein, a compound or pharmaceutical composition, when administered, can ameliorate a disease, symptom, or condition, which particularly refers to ameliorating the severity, delaying the onset, slowing the progression, or reducing the duration of the disease. Fixed or temporary administration, or continuous or intermittent administration, may be attributed to or associated with the administration.

“Active ingredient” refers to the compound of general formula (1), and pharmaceutically acceptable inorganic or organic salts of the compound of general formula (1). The compound of the present disclosure may contain one or more asymmetric centers (chiral center or axial chirality) and thus occurs in the forms of a racemate, a racemic mixture, a single enantiomer, a diastereomeric compound, and a single diastereomer. Asymmetric centers that may be present depend on the nature of the various substituents on the molecule. Each of such asymmetric centers will independently produce two optical isomers, and all possible optical isomers, diastereomeric mixtures and pure or partially pure compounds are included within the scope of the present disclosure. The present disclosure is meant to include all such isomeric forms of these compounds.

The terms such as “compound”, “composition”, “agent”, or “medicine or medicament” are used interchangeably herein and all refer to a compound or composition that, when administered to an individual (human or animal), is capable of inducing a desired pharmacological and/or physiological response by local and/or systemic action.

The term “administered, administering, or administration” refers herein to the direct administration of the compound or composition, or the administration of a prodrug, derivative, analog, or the like of the active compound.

Although the numerical ranges and parameters defining the broad scope of the present disclosure are approximations, the related numerical values set forth in the specific examples have been presented herein as precisely as possible. Any numerical value, however, inherently contains a standard deviation necessarily resulting from certain methods of testing. Herein, “about” generally means that the actual numerical value is within a particular numerical value or range±10%, 5%, 1%, or 0.5%. Alternatively, the term “about” indicates that the actual numerical value falls within the acceptable standard error of a mean, as considered by those skilled in the art. All ranges, quantities, numerical values, and percentages used herein (e.g., to describe an amount of a material, a length of time, a temperature, an operating condition, a quantitative ratio, and the like) are to be understood as being modified by the word “about”, except in the experimental examples or where otherwise explicitly indicated. Accordingly, unless otherwise contrarily stated, the numerical parameters set forth in the specification and the appended claims are all approximations that may vary as desired. At least, these numerical parameters should be understood as the significant digits indicated or the numerical values obtained using conventional rounding rules.

Unless otherwise defined in the specification, the scientific and technical terms used herein have the same meaning as commonly understood by those skilled in the art. Furthermore, nouns in their singular forms used in the specification encompass their plural forms, unless contradicted by context; nouns in their plural forms used also encompass their singular forms.

### Therapeutic Use

The compound of general formula (1) or general formula (2) or the pharmaceutical composition of the present disclosure is generally useful for inhibiting SAE protein, and therefore, for treating one or more disorders related to the activity of SAE protein. Therefore, in certain embodiments, the present disclosure provides a method for treating SAE protein-mediated disorders, which comprises the step of administering to a patient in need thereof the compound of general formula (1) or general formula (2) or the pharmaceutically acceptable composition thereof of the present disclosure.

In some embodiments, a method for treating cancer is provided, the method comprising administering to an individual in need thereof an effective amount of any of the aforementioned pharmaceutical compositions comprising the compound of structural general formula (1) or general formula (2). In some embodiments, the cancer includes but is not limited to hematologic malignancies (leukemia, lymphoma, myeloma including multiple myeloma, myelodysplastic syndrome, and myeloproliferative syndrome) and solid tumors (carcinomas such as prostate, breast, lung, colon, pancreas, kidney, ovary, and soft tissue cancers, osteosarcoma, and interstitial tumors), and the like, preferably lung cancer, cervical cancer, colorectal cancer, lymphoma, myeloma, leukemia, hepatocellular carcinoma, pancreatic cancer, kidney cancer, breast cancer, head and neck cancer, melanoma, prostate cancer, adrenal cancer, endometrial cancer, appendiceal cancer, and metastasis of these cancers.

### Route of Administration

The compound and the pharmaceutically acceptable salt thereof of the present disclosure can be made into various formulations comprising a safe and effective amount of the compound or the pharmaceutically acceptable salt thereof of the present disclosure, and a pharmaceutically acceptable excipient or carrier. The “safe and effective amount” means that the amount of the compound is sufficient to significantly improve the condition without causing serious side effects. The safe and effective amount of the compound is determined according to the age, condition, course of treatment, and other specific conditions of a treated subject.

The “pharmaceutically acceptable excipient or carrier” refers to one or more compatible solid or liquid fillers or gel substances that are suitable for human use and must be of sufficient purity and sufficiently low toxicity.

“Compatible” herein means that the components of the composition are capable of intermixing with the compound of the present disclosure and with each other, without significantly diminishing the pharmaceutical efficacy of the compound. Examples of pharmaceutically acceptable excipients or carriers include cellulose and derivatives thereof (e.g., sodium carboxymethylcellulose, sodium ethylcellulose, or cellulose acetate), gelatin, tale, solid lubricants (e.g., stearic acid or magnesium stearate), calcium sulfate, vegetable oil (e.g., soybean oil, sesame oil, peanut oil, or olive oil), polyols (e.g., propylene glycol, glycerol, mannitol, or sorbitol), emulsifiers (e.g., Tween®), wetting agents (e.g., sodium lauryl sulfate), colorants, flavoring agents, stabilizers, antioxidants, preservatives, pyrogen-free water, etc.

When the compound of the present disclosure is administered, it may be administered orally, rectally, parenterally (intravenously, intramuscularly, or subcutaneously), or topically.

Solid dosage forms for oral administration include capsules, tablets, pills, pulvises, and granules. In these solid dosage forms, the active compound is mixed with at least one conventional inert excipient (or carrier), such as sodium citrate or dicalcium phosphate, or with the following ingredients: (a) fillers or extenders, such as starch, lactose, sucrose, glucose, mannitol, and silicic acid; (b) binders, such as hydroxymethyl cellulose, alginate, gelatin, polyvinylpyrrolidone, sucrose, and acacia; (c) humectants, such as glycerol; (d) disintegrants, such as agar, calcium carbonate, potato or tapioca starch, alginic acid, certain complex silicates, and sodium carbonate; (e) solution retarders, such as paraffin; (f) absorption accelerators, such as quaternary ammonium compounds; (g) wetting agents, such as cetyl alcohol and glycerol monostearate; (h) adsorbents, such as kaolin; and (i) lubricants, such as tale, calcium stearate, magnesium stearate, solid polyethylene glycol and sodium lauryl sulfate, or mixtures thereof. In the case of capsules, tablets, and pills, the dosage forms may further include buffers.

Solid dosage forms such as tablets, dragees, capsules, pills and granules can be prepared using coatings and shells such as enteric coatings and other materials well known in the art. They may include opacifying agents, and the active compound or compound in such a composition may be released in a certain part of the digestive tract in a delayed manner. Examples of embedding components that can be used are polymeric substances and wax-based substances. If necessary, the active compound can also be in microcapsule form with one or more of the excipients described above.

Liquid dosage forms for oral administration include pharmaceutically acceptable emulsions, solutions, suspensions, syrups, and elixirs. In addition to the active compound, the liquid dosage form may include inert diluents commonly used in the art, such as water or other solvents, solubilizers and emulsifiers, for example, ethanol, isopropanol, ethyl carbonate, ethyl acetate, propylene glycol, 1,3-butanediol, dimethylformamide, and oils, especially cottonseed oil, peanut oil, corn germ oil, olive oil, castor oil and sesame oil, or mixtures of these substances.

Besides such inert diluents, the composition may further include adjuvants, such as wetting agents, emulsifiers, suspending agents, sweeteners, flavoring agents, and perfuming agents.

In addition to the active compound, suspensions may include suspending agents, such as ethoxylated isostearyl alcohols, polyoxyethylene sorbitol and sorbitan esters, microcrystalline cellulose, aluminum methylate and agar, or mixtures of these substances.

Compositions for parenteral injection may include physiologically acceptable sterile aqueous or anhydrous solutions, dispersions, suspensions or emulsions, and sterile powders for redissolving into sterile injectable solutions or dispersions. Suitable aqueous and non-aqueous carriers, diluents, solvents, or excipients include water, ethanol, polyols, and suitable mixtures thereof.

Dosage forms for topical administration of the compound of the present disclosure include ointments, pulvises, patches, sprays, and inhalants. The active ingredient is mixed under sterile conditions with a physiologically acceptable carrier and any preservatives, buffers or propellants that may be required if necessary.

The compound of the present disclosure may be administered alone or in combination with other pharmaceutically acceptable compounds. When the pharmaceutical composition is used, a safe and effective amount of the compound of the present disclosure is administered to a mammal (such as a human) to be treated, wherein the dose of administration is a pharmaceutically effective dose. For a human of 60 kg, the daily dose of administration is usually 1-2000 mg, preferably 50-1000 mg. In determining a specific dose, such factors as the route of administration, the health condition of the patient and the like will also be considered, which are well-known to skilled physicians.

The above features mentioned in the present disclosure or those mentioned in the examples may be combined arbitrarily. All the features disclosed in this specification may be used with any composition form and the various features disclosed in this specification may be replaced with any alternative features that provide the same, equivalent, or similar purpose. Thus, unless otherwise specified, the features disclosed herein are merely general examples of equivalent or similar features.

### DETAILED DESCRIPTION

Various specific aspects, features, and advantages of the compounds, methods, and pharmaceutical compositions described above will be set forth in detail in the following description, which will make the content of the present disclosure very clear. It should be understood that the detailed description and examples below describe specific examples for reference only. After reading the description of the present disclosure, those skilled in the art can make various changes or modifications to the present disclosure, and such equivalents also fall within the scope of the present application defined herein.

In all the examples, ¹H-NMR spectra were recorded with a Varian Mercury 400 nuclear magnetic resonance spectrometer, and chemical shifts are represented by 6 (ppm); silica gel for separation was 200-300 mesh silica gel if not specified, and the ratio of the eluents was a volume ratio.

The following abbreviations are used in the present disclosure: (Boc)₂O for di-tert-butyl dicarbonate; CDCl₃ for deuterated chloroform; Cs₂CO₃ for cesium carbonate; EtOAc or Ethyl Acetate for ethyl acetate; Hexane for n-hexane; HPLC for high-performance liquid chromatography; MeCN for acetonitrile; DAST for diethylaminosulfur trifluoride; DCM for dichloromethane; DIAD for diisopropyl azodicarboxylate; DIPEA for diisopropylethylamine; Dioxane for 1,4-dioxane; DME for glycol dimethyl ether; DMF for N,N-dimethylformamide; DMAP for 4-(dimethylamino)pyridine; DMSO for dimethyl sulfoxide; DAST for Diethylaminosulfur Trifluoride or diethylaminosulfur trifluoride; EtOH for ethanol; h for hour; IPA for isopropanol; ISCO® for a Biotage Isolera Prime flash preparative liquid chromatograph; min for minute; K₂CO₃ for potassium carbonate; KOAc for potassium acetate; KOH for potassium hydroxide; K₃PO₄ for potassium phosphate; LiBH₄ for lithium borohydride; LiHMDS for lithium bis(trimethylsilyl)amide; min for minute; MeOH for methanol; MeONa for sodium methoxide; MS for mass spectrometry; NaBH(OAc)₃ for sodium triacetoxyborohydride; NaH for sodium hydrogen; NaN₃ for sodium azide; NH₄Cl for ammonium chloride; n-BuLi for n-butyllithium; NMR for nuclear magnetic resonance; NIS for iodosuccinimide; PBST for a phosphate-buffered saline solution containing Tween; Pd/C for palladium on carbon; Pd(PPh₃)₄ for tetrakis(triphenylphosphine)palladium; Pd(OAc)₂ for palladium acetate; Pd(dppf)Cl₂ for [1,1′-bis(diphenylphosphino)ferrocene]dichloropalladium(II); Pd(dtbpf)Cl₂ for Dichloro(1,1′-bis(di-tert-butylphosphino)ferrocene)palladium(II); PE or Petroleum ether for petroleum ether; PFA for paraformaldehyde; PhNTf₂ for N-Phenylbis(trifluoromethanesulfonimide); PPh₃ for triphenylphosphine; PPTS for pyridinium p-toluenesulfonate; TEA for triethylamine; TFA for trifluoroacetic acid; Ti(i-PrO)₄ for titanium tetraisopropoxide; TMSCl for trimethylchlorosilane; TsOH for p-toluenesulfonic acid; XantPhos for 4,5-bis(diphenylphosphino)-9,9-dimethylxanthene; TfOH for trifluoromethanesulfonic acid; TIPS for triisopropylsilyl; TLC for thin-layer chromatography; and XPhos for 2-dicyclohexylphosphonium-2′,4′,6′-triisopropylbiphenyl.

### Example 1. Synthesis of Compound 1

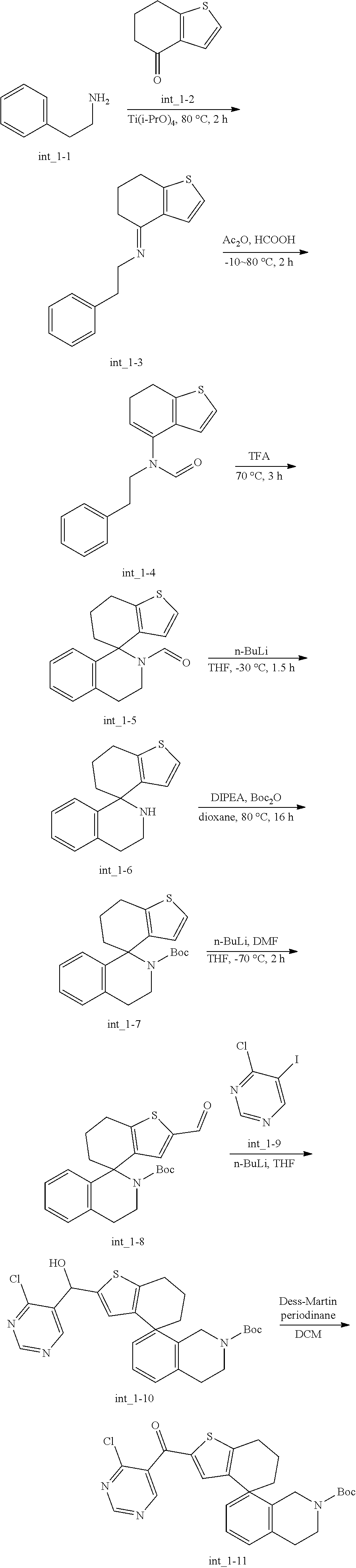

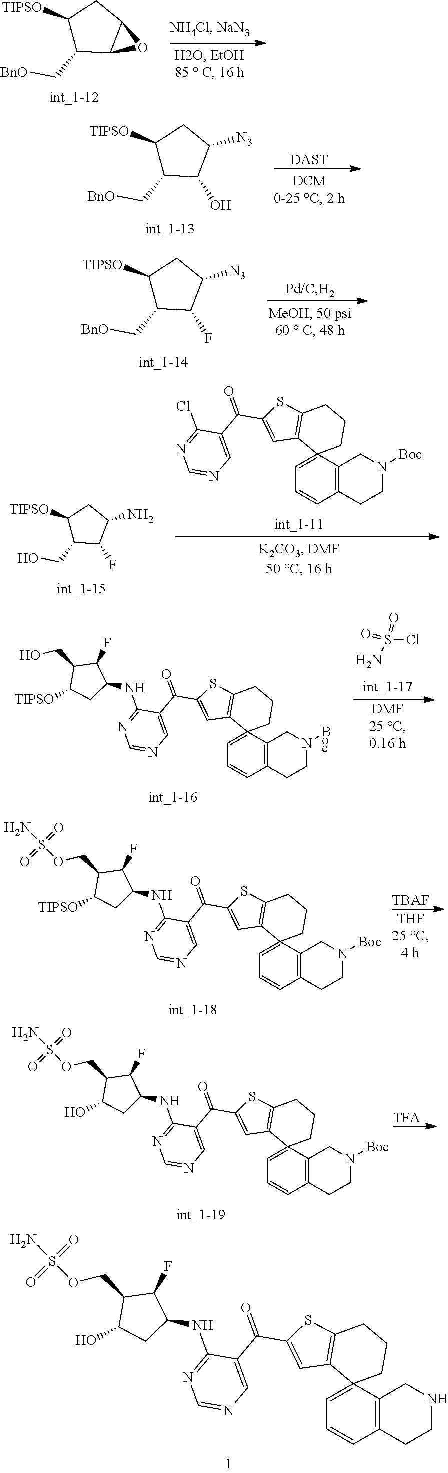

### Step 1: Synthesis of compound int_1-3

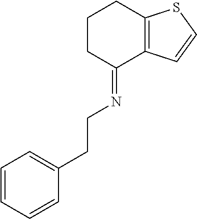 [SMILES: c1ccc(CC/N=C2\CCCc3sccc32)cc1]

Int_1-1 (2.8 g, 22.99 mmol) and int_1-2 (3.5 g, 22.99 mmol, 454.55 μL) were dissolved in Ti(i-PrO)₄ (20 mL). The mixed solution was purged with nitrogen three times, heated to 80° C., and stirred for 2 h. LC-MS monitoring showed the reaction was completed. The reaction solution was cooled to room temperature, which was directly used in the next step.

ESI-MS m/z: 256 [M+H]⁺.

### Step 2: Synthesis of compound int_1-4

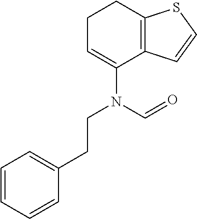 [SMILES: O=CN(CCc1ccccc1)C1=CCCc2sccc21]

HCOOH (78 mL) was slowly and dropwise added to Ac₂O (194 mL) at 0° C., and after the addition was completed, the reaction solution was allowed to react at 20° C. for 0.5 h. Then, the int_1-3 solution obtained in step 1 was cooled to −10° C., and the mixed solution of HOOCH and Ac₂O described above was slowly and dropwise added to the int_1-3 solution. The temperature was kept at −10° C. during the dropwise addition. After the dropwise addition was completed, the reaction solution was warmed to 80° C. and allowed to react for 2 h. LC-MS monitoring showed the reaction was completed. The reaction solution was directly used in the next step.

ESI-MS m/z: 284 [M+H]⁺.

### Step 3: Synthesis of Compound Int_1-5

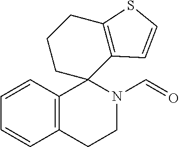 [SMILES: O=CN1CCc2ccccc2C12CCCc1sccc12]

TFA (316 mL) was slowly added to the int_1-4 solution obtained in step 2 at 70° C. within 1 h. The mixed solution was cooled, stirred at 70° C., and allowed to react for 3 h. LC-MS monitoring showed the reaction was completed. After the reaction solution was cooled to room temperature, the reaction solution was concentrated under reduced pressure to give a crude product, and the crude product was adjusted to a weakly basic pH with an aqueous sodium bicarbonate solution (400 mL). The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography (SiO₂, PE/EtOAc=4/1) to give a solid (3.5 g, yield: 48.6%).

ESI-MS m/z: 284 [M+H]⁺.

### Step 4: Synthesis of Compound Int_1-6

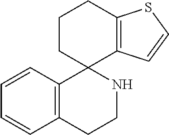 [SMILES: c1ccc2c(c1)CCNC21CCCc2sccc21]

Int_1-5 (1.2 g, 3.83 mmol) was dissolved in THF (20 mL), and the mixture was cooled to −30° C. n-BuLi (2.5 M, 2.30 mL) was slowly and dropwise added to the reaction solution under nitrogen atmosphere, and the reaction solution was allowed to react at −30° C. for 2 h. LC-MS monitoring showed the reaction was completed. After the reaction solution was cooled to room temperature, a saturated aqueous ammonium chloride solution (20 mL) was added to the reaction solution. The aqueous phase was extracted with ethyl acetate (50 mL×3), and the organic phase was dried over anhydrous sodium sulfate. The organic phase was concentrated under reduced pressure to give a crude product, which was directly used in the next step.

ESI-MS m/z: 256 [M+H]⁺.

### Step 5: Synthesis of Compound Int_1-7

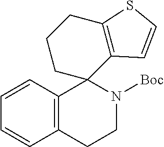 [SMILES: CC(C)(C)OC(=O)N1CCc2ccccc2C12CCCc1sccc12]

Int_1-6 (1.2 g, 3.73 mmol) and (Boc)₂O (1.22 g, 5.59 mmol, 1.28 mL) were dissolved in 1,4-dioxane (20 mL), and TEA (1.13 g, 11.18 mmol, 1.56 mL) was added to the reaction solution at room temperature. The reaction solution was warmed to 90° C. and allowed to react for 16 h. LC-MS monitoring showed the reaction was completed. After the reaction solution was cooled to room temperature, the reaction solution was concentrated under reduced pressure to give a crude product. The crude product was purified by preparative column chromatography (SiO₂, PE/EtOAc=4/1) to give a solid (1.2 g, yield: 83.4%).

ESI-MS m/z: 356 [M+H]⁺.

### Step 6: Synthesis of Compound Int_1-8

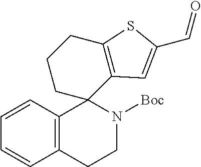 [SMILES: CC(C)(C)OC(=O)N1CCc2ccccc2C12CCCc1sc(C=O)cc12]

Int_1-7 (1.2 g, 3.11 mmol) was dissolved in THF (25 mL), and the mixture was cooled to −70° C. n-BuLi (2.5 M, 3.74 mL) was slowly and dropwise added to the reaction solution under nitrogen atmosphere, and the reaction solution was allowed to react at −70° C. for 0.5 h. Then, DMF (682.55 mg, 9.34 mmol, 718.48 μL) was dropwise added to the reaction solution at −70° C., and after the dropwise addition was completed, the reaction solution was allowed to react at −60° C. for 1.5 h. LC-MS monitoring showed the reaction was completed. A saturated ammonium chloride solution (20 mL) was slowly added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (1.08 g, yield: 83.9%).

MS (ESI): 384 [M+H]⁺.

### Step 7: Synthesis of Compound Int_1-10

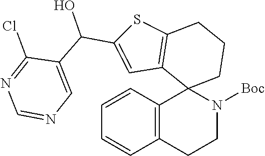 [SMILES: CC(C)(C)OC(=O)N1CCc2ccccc2C12CCCc1sc(C(O)c3cncnc3Cl)cc12]

Int_1-9 (1.50 g, 6.26 mmol) was dissolved in THF (30 mL), and the mixture was cooled to −70° C. n-BuLi (2.5 M, 5.01 mL) was slowly and dropwise added to the reaction solution under nitrogen atmosphere, and the reaction solution was allowed to react at −70° C. for 1 h. Then, a solution of int_1-8 (800 mg, 2.09 mmol) in THF (15 mL) was dropwise added to the reaction solution at −70° C., and after the dropwise addition was completed, the reaction solution was allowed to react at −70° C. for 1 h, and was then warmed to room temperature and allowed to react for 16 h. L C-MS monitoring showed the reaction was completed. A saturated ammonium chloride solution (50 mL) was slowly added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a solid (600 mg, yield: 57.7%).

¹H NMR (400 MHz, CHLOROFORM-d) 6=9.07-9.02 (m, 1H), 8.96-8.91 (m, 1H), 7.19 (d, J=4.4 Hz, 2H), 7.12-7.03 (m, 1H), 6.60-6.50 (m, 2H), 6.24-6.15 (m, 1H), 4.47-4.36 (m, 1H), 3.21 (dt, J=2.6, 12.2 Hz, 1H), 3.10-2.99 (m, 1H), 2.93-2.85 (m, 1H), 2.84-2.66 (m, 4H), 1.85-1.59 (m, 6H), 1.27-1.17 (m, 9H).

MS (ESI): 498 [M+H]⁺.

### Step 8: Synthesis of Compound Int_1-11

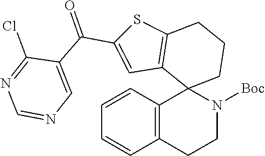 [SMILES: CC(C)(C)OC(=O)N1CCc2ccccc2C12CCCc1sc(C(=O)c3cncnc3Cl)cc12]

Int_1-10 (600 mg, 1.20 mmol) was dissolved in DCM (6 mL), and a Dess-Martin oxidant (766.46 mg, 1.81 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (550 mg, yield: 92%).

MS (ESI): 496 [M+H]⁺.

### Step 9: Synthesis of compound int_1-13

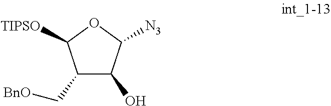

Int_1-12 (23.0 g, 61.1 mmol) and NH₄Cl (11.4 g, 214 mmol) was dissolved in water (100 mL) and ethanol (400 mL). NaN₃ (11.9 g, 183 mmol) was slowly added to the reaction solution at room temperature, and the reaction solution was warmed to 85° C. and allowed to react for 16 h under nitrogen atmosphere. TLC monitoring showed the reaction was completed. A saturated aqueous sodium carbonate solution was slowly added to the reaction solution to adjust the pH to >9. The aqueous phase was extracted with ethyl acetate (500 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-15% THF/Petroleum ether gradient) to give a oily liquid (17.3 g, yield: 67.5%).

¹H NMR (400 MHz, DMSO-d6) δ=7.41-7.23 (m, 5H), 5.46 (d, J=6.1 Hz, 1H), 4.55-4.39 (m, 2H), 4.17-4.10 (m, 1H), 3.88-3.75 (m, 1H), 3.69-3.60 (m, 1H), 3.52 (dq, J=4.3, 9.1 Hz, 2H), 1.93-1.77 (m, 2H), 1.71 (ddd, J=6.8, 9.1, 13.3 Hz, 1H), 1.08-0.90 (m, 22H).

### Step 10: Synthesis of Compound Int_1-14

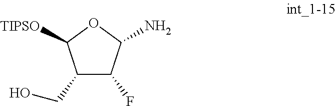

DAST (2.88 g, 17.9 mmol, 2.36 mL) was dissolved in DCM (30 mL). Under nitrogen atmosphere, a solution (30 mL) of int_1-13 (5.00 g, 11.9 mmol) in DCM was added to the reaction solution in an ice bath, and the reaction solution was allowed to react at room temperature for 2 h under nitrogen atmosphere. TLC monitoring showed the reaction was completed. A saturated aqueous sodium bicarbonate solution was slowly added to the reaction solution to adjust the pH to about 8. The aqueous phase was extracted with dichloromethane (100 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-10% THF/Petroleum ether gradient) to give a oily liquid (2.70 g, yield: 53.7%).

¹H NMR (400 MHz, DMSO-d6) δ=7.41-7.24 (m, 5H), 4.52-4.48 (m, 2H), 4.47-4.30 (m, 1H), 4.18-4.07 (m, 1H), 3.56-3.49 (m, 2H), 2.71-2.56 (m, 1H), 2.46-1.93 (m, 2H), 1.77-1.41 (m, 1H), 1.05-0.95 (m, 24H).

### Step 11: Synthesis of Compound Int_1-15

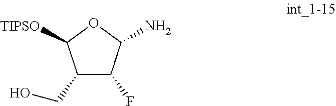

Int_1-14 (2.70 g, 6.40 mmol) was dissolved in methanol (40 mL), and Pd/C (1.50 g, 10% purity) was added to the reaction solution at room temperature. The reaction solution was purged with hydrogen three times, and then warmed to 60° C. and allowed to react under hydrogen atmosphere (50 PSI) for 48 h. LC-MS monitoring showed the reaction was completed. The reaction solution was cooled to room temperature and filtered, and the filtrate was collected. The filtrate was concentrated under reduced pressure to give a crude product (500 mg, yield: 25.6%), which was directly used in the next step.

MS (ESI): 306 [M+H]⁺.

### Step 12: Synthesis of Compound Int_1-16

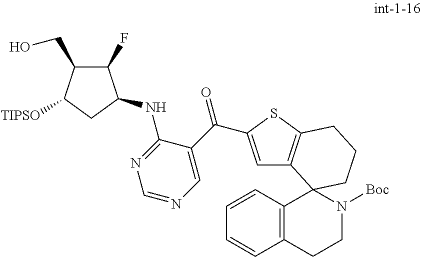

Int_1-15 (0.500 g, 1.64 mmol) and int_1-11 (813 mg, 1.64 mmol) were dissolved in DMF (10 mL), and K₂CO₃ (679 mg, 4.91 mmol) was added to the reaction solution at room temperature. The reaction solution was warmed to 50° C. and allowed to react for 1 h. LC-MS monitoring showed the reaction was completed. Ice water (100 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-30% THF/Petroleum ether gradient) to give a product (382 mg, yield: 30.4%).

MS (ESI): 765 [M+H]⁺.

### Step 13: Synthesis of Compound Int_1-18

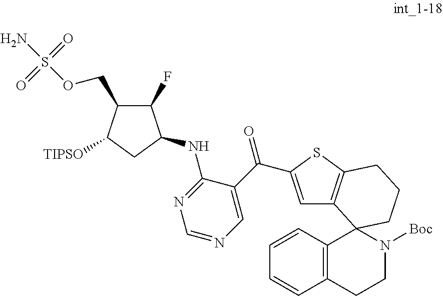

Int_1-16 (0.178 g, 233 μmol) was dissolved in DMF (2 mL), and int_1-17 (269 mg, 2.33 mmol) was added to the reaction solution at room temperature. The reaction solution was allowed to react at room temperature for 1 h. LC-MS monitoring showed the reaction was completed. Ice water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography to give a solid (165 mg, yield: 84.1%).

MS (ESI): 844 [M+H]⁺.

### Step 14: Synthesis of Compound Int_1-19

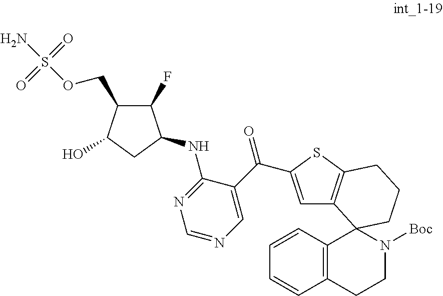 [SMILES: CC(C)(C)OC(=O)N1CCc2ccccc2C12CCCc1sc(C(=O)c3cncnc3N[C@H]3C[C@H](O)[C@@H](COS(N)(=O)=O)[C@H]3F)cc12]

Int_1-18 (0.500 g, 592 μmol) was dissolved in THF (5 mL), and TBAF (1 M, 1.17 mL) was added to the reaction solution at room temperature. The reaction solution was allowed to react at room temperature for 1 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-100% Ethyl acetate/Petroleum ether gradient) to give a solid (190 mg, yield: 46.6%).

MS (ESI): 688 [M+H]⁺.

### Step 15: Synthesis of Compound 1

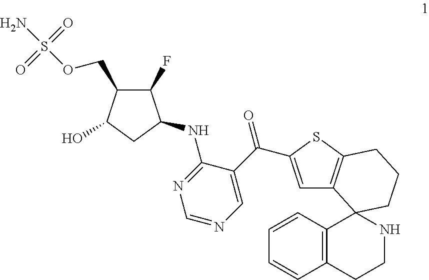 [SMILES: NS(=O)(=O)OC[C@H]1[C@@H](F)[C@@H](Nc2ncncc2C(=O)c2cc3c(s2)CCCC32NCCc3ccccc32)C[C@@H]1O]

Int_1-16 (500 mg, 726 μmol) was added to TFA (1 mL) at room temperature, and the reaction solution was allowed to react at room temperature for 5 min. LC-MS monitoring showed the reaction was completed. A saturated aqueous sodium bicarbonate solution was added to the reaction solution to adjust the pH to 8, and then the reaction solution was purified by preparative HPLC (column: Boston Prime C18 150×30 mm×5 μm; mobile phase: [water (ammonia hydroxide v/v)-ACN]; B %: 30%-50%, 9 min) to give a solid (118 mg, yield: 27.6%).

MS (ESI): 588 [M+H]⁺.

### Example 2. Synthesis of Compound 2 and Compound 3

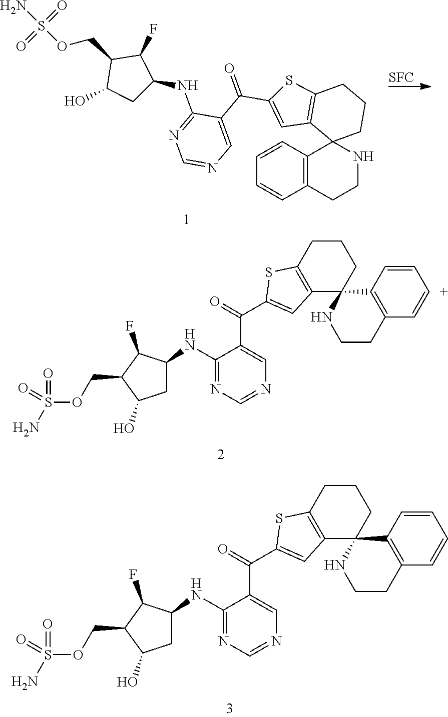

Compound 1 (0.1 g, 170 μmol) was subjected to SFC chiral resolution (column: DAICEL CHIRALPAK IG (250 mm×30 mm, 10 μm); mobile phase: [CO₂-EtOH (0.1% NH₃H₂O)]) to give compound 2 (peak 2, 33 mg) and compound 3 (peak 1, 31 mg).

Compound 2: MS (ESI): 588 [M+H]⁺.

Compound 3: MS (ESI): 588 [M+H]⁺

### Example 3. Synthesis of Compound 88 (Synthetic Route 1)

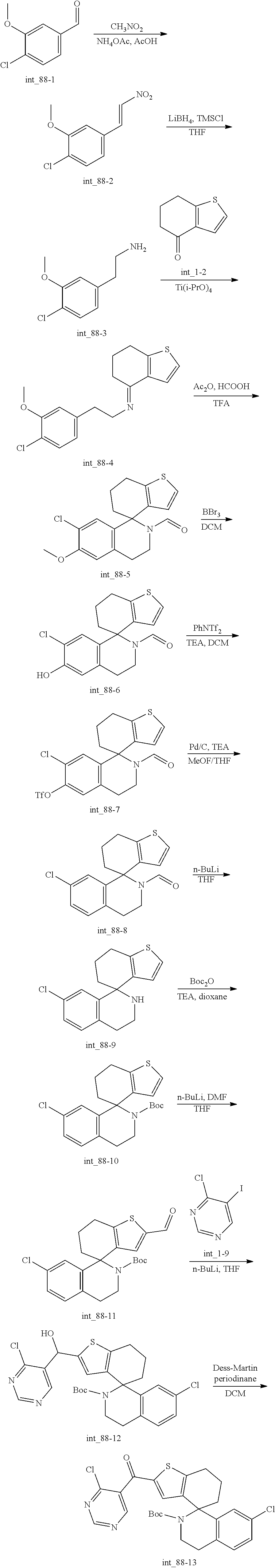

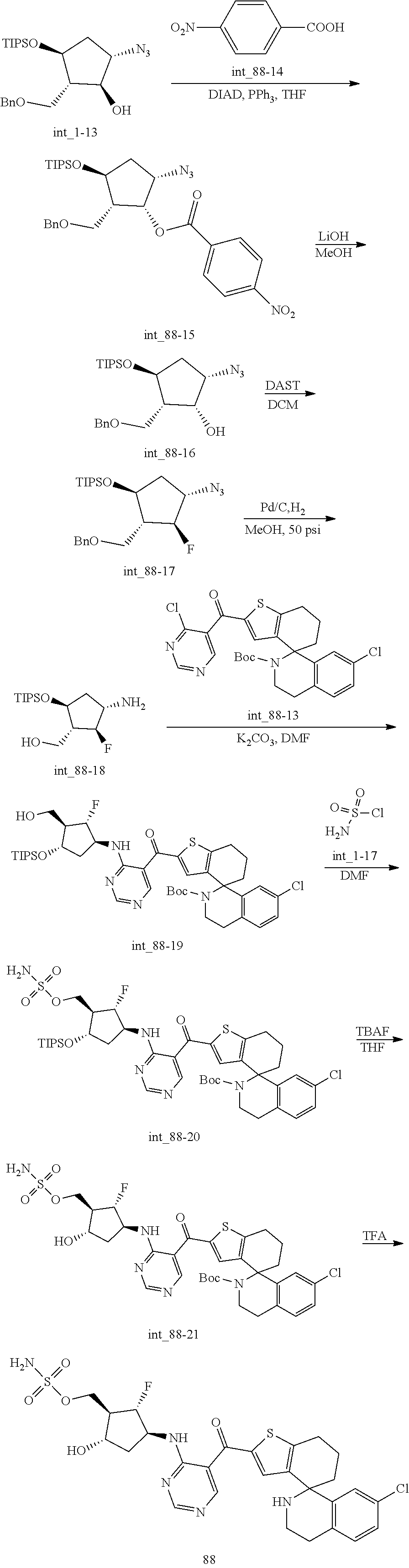

### Step 1: Synthesis of Compound Int_88-2

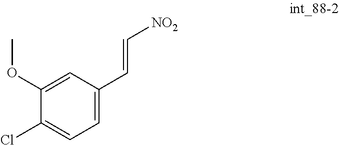

Int_88-1 (25.0 g, 162 mmol) and NH₄OAc (28.8 g, 373 mmol) were dissolved in acetic acid (250 mL), and the mixed solution was purged with nitrogen three times. CH₃NO₂ (25.7 g, 422 mmol, 22.8 mL) was added to the reaction solution under nitrogen atmosphere, and the reaction solution was heated to 100° C. and stirred for 16 h. LC-MS monitoring showed the reaction was completed. The reaction solution was cooled to room temperature and then poured slowly into 1000 mL of ice water. The aqueous phase was extracted with ethyl acetate (800 mL×3), and the organic phase was dried over anhydrous sodium sulfate. The organic phase was concentrated under reduced pressure to give a crude product (27 g, yield: 78.2%), which was directly used in the next step. ¹HNMR (400 MHz, DMSO-d6) δ=3.89 (s, 3H) 7.33 (dd, J=11.25, 8.44 Hz, 1H) 7.46 (ddd, J=8.25, 4.59, 1.96 Hz, 1H) 7.72 (dd, J=8.44, 1.96 Hz, 1H) 8.10 (d, J=13.57 Hz, 1H) 8.29 (d, J=13.57 Hz, 1H).

### Step 2: Synthesis of Compound Int_88-3

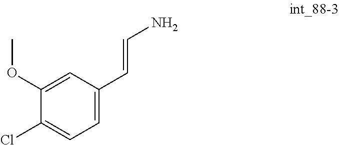

LiBH₄ (27.6 g, 1.27 mol) was dissolved in tetrahydrofuran (300 mL), and TMSCl (276 g, 2.54 mol, 322 mL) was added to the reaction solution at 0° C. under nitrogen atmosphere. The reaction solution was stirred at 0° C. for 30 min, and a solution of int_88-2 (25.0 g, 127 mmol) in tetrahydrofuran (250 mL) was slowly and dropwise added to the reaction solution within 30 min. The reaction solution was warmed to 75° C. and allowed to react for another 2 h. LC-MS monitoring showed the reaction was completed. The reaction solution was cooled to 0° C., and 600 mL of methanol was slowly added to quench the reaction. The organic phase was concentrated under reduced pressure to give a crude product, and the crude product was adjusted to pH>7 with ammonia water. The aqueous phase was extracted with ethyl acetate (500 mL×3), and the organic phase was dried over anhydrous sodium sulfate. The organic phase was concentrated under reduced pressure to give a crude product (25 g), which was directly used in the next step.

ESI-MS m/z: 170 [M+H]⁺.

### Step 3: Synthesis of Compound Int_88-4

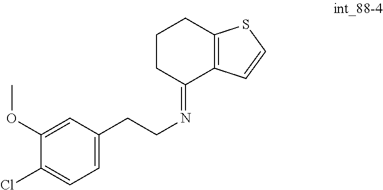 [SMILES: COc1cc(CC/N=C2\CCCc3sccc32)ccc1Cl]

Int_88-3 (12.5 g, 73.9 mmol) and int_1-2 (11.8 g, 77.6 mmol) were dissolved in Ti(i-PrO)₄ (1130 mL). The mixed solution was purged with nitrogen three times, heated to 80° C., and stirred for 2 h. LC-MS monitoring showed the reaction was completed. The reaction solution was cooled to room temperature, which was directly used in the next step.

ESI-MS m/z: 304 [M+H]⁺.

### Step 4: Synthesis of Compound Int_88-5

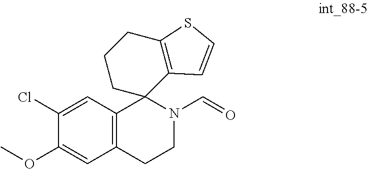 [SMILES: COc1cc2c(cc1Cl)C1(CCCc3sccc31)N(C=O)CC2]

HCOOH (200 mL) was slowly and dropwise added to Ac₂O (500 mL) at −10° C., and after the addition was completed, the reaction solution was allowed to react at 20° C. for 0.5 h. Then, the int_88-4 solution obtained in step 3 was cooled to −10° C., and the mixed solution of HOOCH and Ac₂O described above was slowly and dropwise added to the int_88-4 solution. The temperature was kept at −10° C. during the dropwise addition. After the dropwise addition was completed, the reaction solution was warmed to 70° C. and allowed to react for 3 h. LC-MS monitoring showed the reaction was completed. The reaction solution was cooled to room temperature. The organic phase was concentrated under reduced pressure to give a crude product, and the crude product was adjusted to pH>7 with a saturated aqueous sodium bicarbonate solution. The aqueous phase was extracted with ethyl acetate (500 mL×3), and the organic phase was dried over anhydrous sodium sulfate. The organic phase was concentrated under reduced pressure to give a crude product. The crude product was purified by preparative column chromatography (SiO₂, PE/EtOAc=1/1) to give a solid (2 g, yield: 10.6%).

¹H NMR (400 MHz, DMSO-d6) δ=7.57 (s, 1H), 7.32 (d, J=5.1 Hz, 1H), 6.98 (s, 1H), 6.72 (s, 1H), 6.24 (d, J=5.3 Hz, 1H), 4.40 (td, J=3.9, 12.9 Hz, 1H), 3.85 (s, 3H), 3.24-3.12 (m, 1H), 2.93 (br dd, J=3.7, 7.9 Hz, 2H), 2.89-2.79 (m, 2H), 2.35 (br dd, J=2.0, 11.9 Hz, 1H), 2.01-1.81 (m, 2H), 1.70-1.53 (m, 1H).

ESI-MS m/z: 348 [M+H]⁺.

### Step 5: Synthesis of Compound Int_88-6

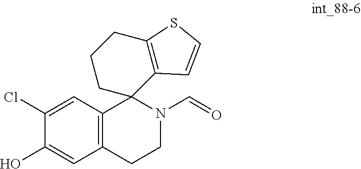 [SMILES: O=CN1CCc2cc(O)c(Cl)cc2C12CCCc1sccc12]

Int_88-5 (3.30 g, 9.49 mmol) was dissolved in dichloromethane (30 mL), and the mixed solution was purged with nitrogen three times. The reaction solution was cooled to 0° C., and BBr₃ (11.88 g, 47.43 mmol, 4.57 mL) was slowly and dropwise added to the reaction solution. The reaction solution was warmed to room temperature and allowed to react for 1 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product (2.8 g, yield: 88.4%), which was directly used in the next step.

¹H NMR (400 MHz, DMSO-d6) δ=10.36 (s, 1H), 7.72 (s, 1H), 7.48 (d, J=5.1 Hz, 1H), 6.91 (s, 1H), 6.79 (s, 1H), 6.41 (d, J=5.3 Hz, 1H), 4.57-4.45 (m, 1H), 3.38-3.28 (m, 1H), 3.15-3.04 (m, 2H), 2.98-2.85 (m, 2H), 2.55-2.44 (m, 1H), 2.14-1.97 (m, 2H), 1.86-1.66 (m, 1H).

### Step 6: Synthesis of Compound Int_88-7

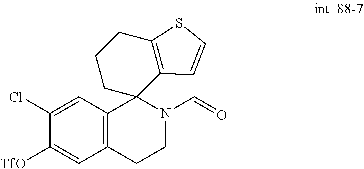 [SMILES: O=CN1CCc2cc(OS(=O)(=O)C(F)(F)F)c(Cl)cc2C12CCCc1sccc12]

Int_88-6 (2.80 g, 8.39 mmol), PhNTf₂ (5.39 g, 15.10 mmol), and TEA (2.12 g, 20.97 mmol, 2.92 mL) were dissolved in dichloromethane (30 mL). The reaction solution was purged with nitrogen three times, and allowed to react at room temperature for 16 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography (SiO₂, PE/EtOAc=1/1) to give a solid (2.3 g, yield: 58.8%).

¹H NMR (400 MHz, DMSO-d6) δ=7.45-7.39 (m, 1H), 7.36 (d, J=5.3 Hz, 1H), 7.31-7.24 (m, 1H), 7.12 (s, 1H), 6.28 (d, J=5.3 Hz, 1H), 4.51-4.38 (m, 1H), 3.02-2.85 (m, 4H), 2.42 (td, J=2.0, 12.0 Hz, 2H), 2.06-1.94 (m, 1H), 1.90 (br d, J=13.8 Hz, 1H), 1.94-1.83 (m, 1H), 1.68-1.50 (m, 1H).

ESI-MS m/z: 466 [M+H]⁺.

### Step 7: Synthesis of Compound Int_88-8

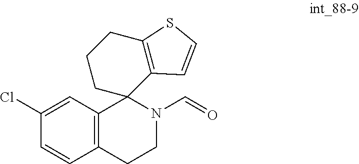 [SMILES: O=CN1CCc2ccc(Cl)cc2C12CCCc1sccc12]

Int_88-7 (2.30 g, 4.94 mmol), Pd/C (1.05 g, 987.35 μmol, 10% purity), and TEA (2.00 g, 19.75 mmol, 2.75 mL) were dissolved in a mixed solvent of methanol (21 mL) and tetrahydrofuran (7 mL). The reaction solution was purged with hydrogen three times, and allowed to react at room temperature for 16 h. LC-MS monitoring showed the reaction was completed. The reaction solution was filtered to give a filtrate, and the filtrate was concentrated under reduced pressure to give a crude product. The crude product was purified by preparative column chromatography (SiO₂, PE/EtOAc=1/1) to give a solid (0.64 g, yield: 40.7%).

¹H NMR (400 MHz, DMSO-d6) δ=7.57 (s, 1H), 7.34 (d, J=5.3 Hz, 1H), 7.25 (d, J=0.9 Hz, 2H), 6.76 (s, 1H), 6.24 (d, J=5.3 Hz, 1H), 4.41 (td, J=4.1, 13.1 Hz, 1H), 3.25-3.13 (m, 1H), 2.95 (dd, J=3.9, 7.8 Hz, 2H), 2.86 (dd, J=4.5, 8.3 Hz, 2H), 2.45-2.35 (m, 1H), 2.03-1.95 (m, 1H), 1.95-1.83 (m, 1H), 1.69-1.51 (m, 1H).

ESI-MS m/z: 318 [M+H]⁺.

### Step 8: Synthesis of Compound Int_88-9

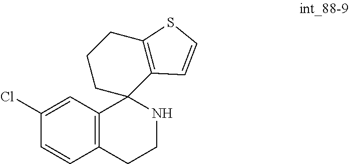 [SMILES: Clc1ccc2c(c1)C1(CCCc3sccc31)NCC2]

Int_88-8 (0.64 g, 2.01 mmol) was dissolved in THF (10 mL), and the mixture was cooled to −30° C. n-BuLi (2.5 M, 2.42 mL) was slowly and dropwise added to the reaction solution under nitrogen atmosphere, and the reaction solution was allowed to react at −30° C. for 2 h. Subsequently, the reaction solution was warmed to room temperature and allowed to react for 1 h. LC-MS monitoring showed the reaction was completed. A saturated aqueous ammonium chloride solution (10 mL) was added to the reaction solution. The aqueous phase was extracted with ethyl acetate (30 mL×3), and the organic phase was dried over anhydrous sodium sulfate. The organic phase was concentrated under reduced pressure to give a crude product (0.5 g, yield: 85.6%), which was directly used in the next step.

¹H NMR (400 MHz, DMSO-d6) δ 7.15-7.10 (m, 3H), 6.69 (t, J=1.3 Hz, 1H), 6.35 (d, J=5.2 Hz, 1H), 3.03-2.93 (m, 2H), 2.91-2.78 (m, 3H), 2.66 (dt, J=16.1, 3.7 Hz, 1H), 2.16 (ddd, J=13.2, 5.0, 2.6 Hz, 1H), 2.03 (ddtd, J=16.0, 13.0, 6.8, 2.7 Hz, 1H), 1.84-1.71 (m, 2H).

ESI-MS m/z: 290 [M+H]⁺.

### Step 9: Synthesis of Compound Int_88-10

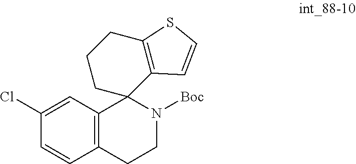 [SMILES: CC(C)(C)OC(=O)N1CCc2ccc(Cl)cc2C12CCCc1sccc12]

Int_88-9 (0.50 g, 1.73 mmol) and (Boc)₂O (828.34 mg, 3.80 mmol, 871.93 μL) were dissolved in 1,4-dioxane (10 mL), and TEA (523.71 mg, 5.18 mmol, 720.37 μL) was added to the reaction solution at room temperature. The reaction solution was warmed to 80° C. and allowed to react for 16 h. LC-MS monitoring showed the reaction was completed. After the reaction solution was cooled to room temperature, the reaction solution was concentrated under reduced pressure to give a crude product. The crude product was purified by preparative column chromatography (SiO₂, PE/EtOAc=10/1) to give a solid (0.11 g, yield: 16.3%).

¹H NMR (400 MHz, DMSO-d6) δ=7.33-7.23 (m, 3H), 6.53 (d, J=5.1 Hz, 1H), 6.40 (d, J=2.0 Hz, 1H), 4.27 (td, J=3.7, 12.0 Hz, 1H), 3.11 (ddd, J=5.1, 9.8, 12.2 Hz, 1H), 2.97-2.87 (m, 3H), 2.75-2.63 (m, 2H), 1.77 (br d, J=13.2 Hz, 1H), 1.65-1.49 (m, 2H), 1.15 (s, 9H).

### Step 10: Synthesis of Compound Int_88-11

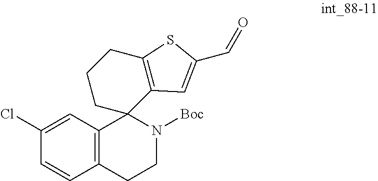 [SMILES: CC(C)(C)OC(=O)N1CCc2ccc(Cl)cc2C12CCCc1sc(C=O)cc12]

Int_88-10 (1.44 g, 3.693 mmol) was dissolved in THF (20 mL), and the mixture was cooled to −75° C. n-BuLi (2.5 M, 4.43 mL) was slowly and dropwise added to the reaction solution under nitrogen atmosphere, and the reaction solution was allowed to react at −75° C. for 1 h. Then, DMF (809 mg, 11.079 mmol) was dropwise added to the reaction solution at −75° C., and after the dropwise addition was completed, the reaction solution was allowed to react at −75° C. for 1 h. LC-MS monitoring showed the reaction was completed. A saturated ammonium chloride solution (15 mL) was slowly added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a solid (1.38 g, yield: 89.9%).

MS (ESI): 418 [M+H]⁺.

### Step 11: Synthesis of Compound Int_88-12

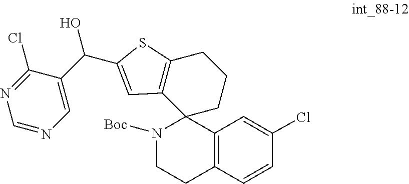 [SMILES: CC(C)(C)OC(=O)N1CCc2ccc(Cl)cc2C12CCCc1sc(C(O)c3cncnc3Cl)cc12]

Int_1-9 (4.79 g, 19.911 mmol) was dissolved in THF (50 mL), and the mixture was cooled to −75° C. n-BuLi (2.5 M, 16 mL) was slowly and dropwise added to the reaction solution under nitrogen atmosphere, and the reaction solution was allowed to react at −75° C. for 1 h. Then, a solution of int_88-11 (1.387 g, 3.319 mmol) in TIF (25 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (200 mL) was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (1.427 g, yield: 80%).

MS (ESI): 532 [M+H]⁺.

### Step 12: Synthesis of Compound Int_88-13

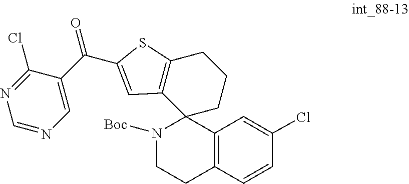 [SMILES: CC(C)(C)OC(=O)N1CCc2ccc(Cl)cc2C12CCCc1sc(C(=O)c3cncnc3Cl)cc12]

Int_88-12 (1.427 g, 2.68 mmol) was dissolved in DCM (50 mL), and a Dess-Martin oxidant (1.36 g, 3.22 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (1.324 g, yield: 93%).

MS (ESI): 530 [M+H]⁺.

### Step 13: Synthesis of Compound Int_88-15

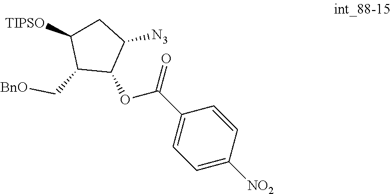

Int_1-13 (5 g, 11.91 mmol), triphenylphosphine (4.68 g, 17.87 mmol), and int_88-14 (2.38 g, 14.29 mmol) were dissolved in THF (200 mL), and DIAD (3.61 g, 17.87 mmol) was added to the reaction solution at room temperature. The reaction solution was allowed to react at room temperature for 6 h. LC-MS monitoring showed the reaction was completed. 200 mL of water was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (5.1 g, yield: 76.1%).

MS (ESI): 569 [M+H]⁺.

### Step 14: Synthesis of Compound Int_88-16

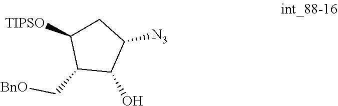

Int_88-15 (5 g, 8.79 mmol) was dissolved in a mixed solvent of methanol (100 mL) and water (50 mL). Lithium hydroxide (631 mg, 26.37 mmol) was added to the reaction solution at room temperature, and the reaction solution was allowed to react at room temperature for 6 h. LC-MS monitoring showed the reaction was completed. 200 mL of water was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (3.2 g, yield: 88.8%).

MS (ESI): 420 [M+H]⁺.

### Step 15: Synthesis of Compound Int_88-17

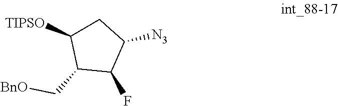

DAST (2.88 g, 17.9 mmol, 2.36 mL) was dissolved in DCM (30 mL). Under nitrogen atmosphere, a solution (30 mL) of int_88-16 (5.00 g, 11.9 mmol) in DCM was added to the reaction solution in an ice bath, and the reaction solution was allowed to react at room temperature for 2 h under nitrogen atmosphere. TLC monitoring showed the reaction was completed. A saturated aqueous sodium bicarbonate solution was slowly added to the reaction solution to adjust the pH to about 8. The aqueous phase was extracted with dichloromethane (100 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-10% THF/Petroleum ether gradient) to give a oily liquid (3.5 g, yield: 70%).

MS (ESI): 422 [M+H]⁺.

### Step 16: Synthesis of Compound Int_88-18

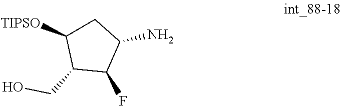

Int_88-17 (3 g, 7.11 mmol) was dissolved in methanol (40 mL), and Pd/C (1.50 g, 10% purity) was added to the reaction solution at room temperature. The reaction solution was purged with hydrogen three times, and then warmed to 60° C. and allowed to react under hydrogen atmosphere (50 PSI) for 48 h. LC-MS monitoring showed the reaction was completed. The reaction solution was cooled to room temperature and filtered, and the filtrate was collected. The filtrate was concentrated under reduced pressure to give a crude product (650 mg, yield: 29.9%), which was directly used in the next step.

MS (ESI): 306 [M+H]⁺.

### Step 17: Synthesis of Compound Int_88-19

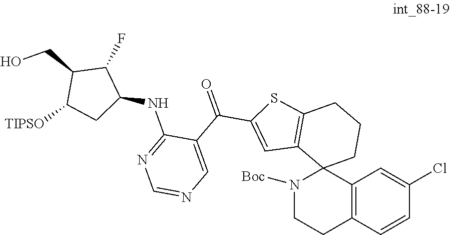

Int_88-18 (501 mg, 1.64 mmol) and int_88-13 (869.9 mg, 1.64 mmol) were dissolved in DMF (10 mL), and K₂CO₃ (679 mg, 4.91 mmol) was added to the reaction solution at room temperature. The reaction solution was warmed to 50° C. and allowed to react for 1 h. LC-MS monitoring showed the reaction was completed. Ice water (100 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-30% THF/Petroleum ether gradient) to give a product (355 mg, yield: 27.3%).

MS (ESI): 799 [M+H]⁺.

### Step 18: Synthesis of Compound Int_88-20

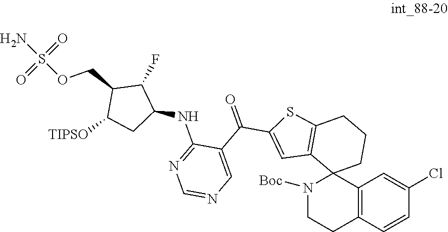

Int_88-19 (186 mg, 233 μmol) was dissolved in DMF (2 mL), and int_1-17 (269 mg, 2.33 mmol) was added to the reaction solution at room temperature. The reaction solution was allowed to react at room temperature for 1 h. LC-MS monitoring showed the reaction was completed. Ice water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography to give a solid (182 mg, yield: 89.2%).

MS (ESI): 878 [M+H]⁺.

### Step 19: Synthesis of Compound Int_88-21

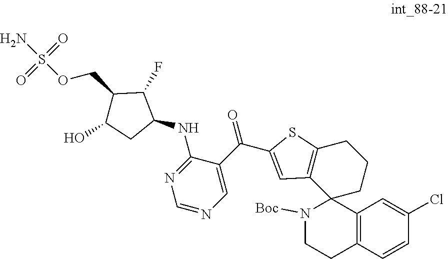 [SMILES: CC(C)(C)OC(=O)N1CCc2ccc(Cl)cc2C12CCCc1sc(C(=O)c3cncnc3N[C@H]3C[C@H](O)[C@@H](COS(N)(=O)=O)[C@@H]3F)cc12]

Int_88-20 (0.500 g, 569 μmol) was dissolved in THF (5 mL), and TBAF (1 M, 1.17 mL) was added to the reaction solution at room temperature. The reaction solution was allowed to react at room temperature for 1 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-100% Ethyl acetate/Petroleum ether gradient) to give a solid (359 mg, yield: 87.3%).

MS (ESI): 722 [M+H]⁺.

### Step 20: Synthesis of Compound 88

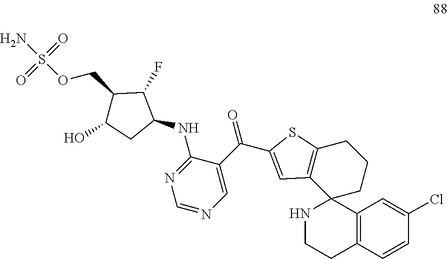 [SMILES: NS(=O)(=O)OC[C@H]1[C@H](F)[C@@H](Nc2ncncc2C(=O)c2cc3c(s2)CCCC32NCCc3ccc(Cl)cc32)C[C@@H]1O]

Int_88-21 (500 mg, 692 μmol) was added to TFA (1 mL) at room temperature, and the reaction solution was allowed to react at room temperature for 5 min. LC-MS monitoring showed the reaction was completed. A saturated aqueous sodium bicarbonate solution was added to the reaction solution to adjust the pH to 8, and then the reaction solution was purified by preparative HPLC (column: Boston Prime C18 150×30 mm×5 μm; mobile phase: [water (ammonia hydroxide v/v)-ACN]; B %: 30%-50%, 9 min) to give a solid (405 mg, yield: 94.1%).

MS (ESI): 622 [M+H]⁺.

### Example 4. Synthesis of Compound 89 and Compound 90

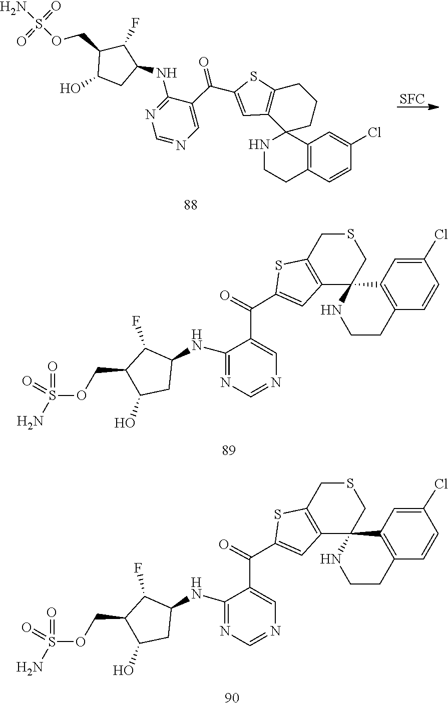 [SMILES: NS(=O)(=O)OC[C@H]1[C@H](F)[C@@H](Nc2ncncc2C(=O)c2cc3c(s2)CCCC32NCCc3ccc(Cl)cc32)C[C@@H]1O ; "SFC" ; NS(=O)(=O)OC[C@H]1[C@H](F)[C@@H](Nc2ncncc2C(=O)c2cc3c(s2)CSC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)CSC[C@]32NCCc3ccc(Cl)cc32)C[C@@H]1O]

Compound 88 (0.1 g, 160 μmol) was subjected to SFC chiral resolution (column: DAICEL CHIRALPAK IG (250 mm×30 mm, 10 μm); mobile phase: [CO₂-EtOH (0.1% NH₃H₂O)]; B %: 55%, isocratic elution mode) to give compound 89 (33 mg) and compound 90 (35 mg).

Compound 89: ¹H NMR (400 MHz, DMSO-d6) δ 8.55 (s, 1H), 8.48 (s, 1H), 8.39 (d, J=8.2 Hz, 1H), 7.50 (s, 2H), 7.14 (d, J=2.5 Hz, 2H), 6.97 (s, 1H), 6.77 (d, J=1.9 Hz, 1H), 5.16 (d, J=4.6 Hz, 1H), 5.02 (dt, J=18.5, 8.3 Hz, 1H), 4.87 (dt, J=54.3, 6.6 Hz, 1H), 4.11 (qd, J=10.0, 5.9 Hz, 2H), 3.91 (t, J=5.4 Hz, 1H), 2.97 (m, 4H), 2.92-2.80 (m, 1H), 2.67 (d, J=16.0 Hz, 1H), 2.31-2.13 (m, 2H), 2.06 (d, J=13.9 Hz, 1H), 1.95 (d, J=10.3 Hz, 1H), 1.90-1.72 (m, 3H); MS (ESI): 622 [M+H]⁺.

Analytical SFC retention time: 1.048 min (Instrument: Waters UPCC with PDA Detector; Column: Chiralpak IG-3 50×4.6 mm I.D., 3 μm; Mobile phase: A: CO₂, B: Ethanol (0.05% DEA); Isocratic: 40% B; Flow rate: 4 mL/min; Column temp: 35° C.; ABPR: 1500 psi).

Compound 90: ¹H NMR (400 MHz, DMSO-d6) δ 8.55 (s, 1H), 8.46 (s, 1H), 8.33 (d, J=8.2 Hz, 1H), 7.51 (s, 2H), 7.12 (t, J=1.6 Hz, 2H), 6.93 (s, 1H), 6.75 (d, J=1.8 Hz, 1H), 5.15 (d, J=4.6 Hz, 1H), 5.01 (dt, J=18.6, 8.4 Hz, 1H), 4.86 (dt, J=54.3, 6.7 Hz, 1H), 4.20-4.01 (m, 2H), 3.93-3.83 (m, 1H), 3.06-2.90 (m, 4H), 2.85 (dt, J=16.1, 7.9 Hz, 1H), 2.63 (d, J=16.2 Hz, 1H), 2.32-2.13 (m, 2H), 2.05 (d, J=8.5 Hz, 1H), 1.93 (t, J=11.1 Hz, 1H), 1.79 (dt, J=14.3, 8.9 Hz, 3H); MS (ESI): 622 [M+H]⁺.

Analytical SFC retention time: 0.592 min (Instrument: Waters UPCC with PDA Detector; Column: Chiralpak IG-3 50x4.6 mm I.D., 3 um; Mobile phase: A: CO₂, B: Ethanol (0.05% DEA); Isocratic: 40% B; Flow rate: 4 mL/min; Column temp: 35° C.; ABPR: 1500 psi).

### Example 5. Synthesis of Intermediate Int_88-9

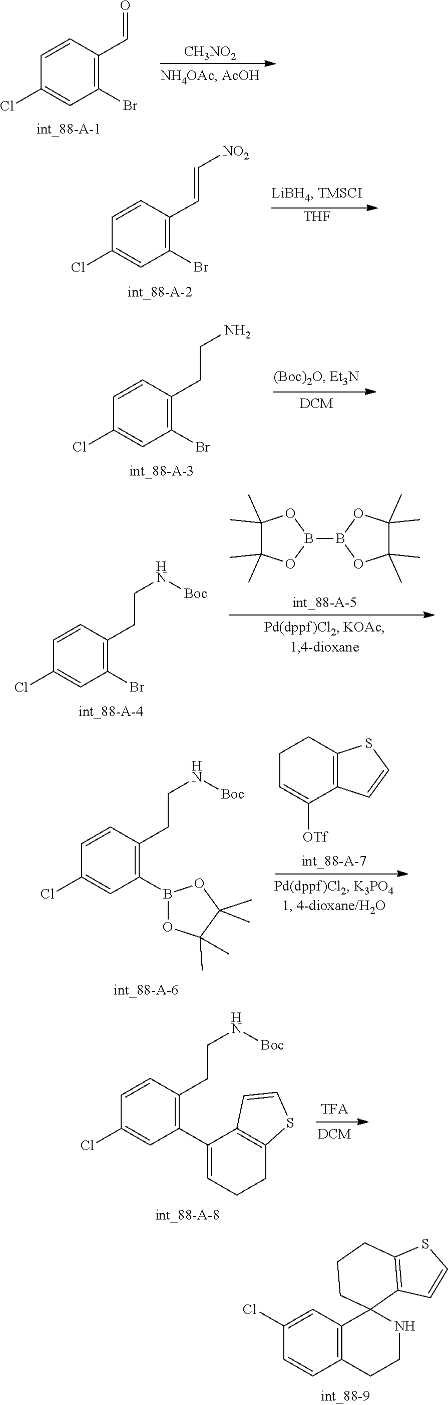

### Step 1: Synthesis of Compound Int_88-A-2

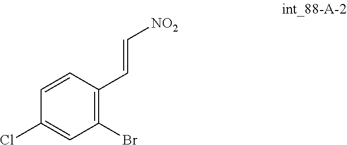 [SMILES: O=[N+]([O-])/C=C/c1ccc(Cl)cc1Br]

Int_88-A-1 (35.0 g, 160 mmol) and NH₄OAc (28.8 g, 373 mmol) were dissolved in acetic acid (250 mL), and the mixed solution was purged with nitrogen three times. CH₃NO₂ (25.7 g, 422 mmol, 22.8 mL) was added to the reaction solution under nitrogen atmosphere, and the reaction solution was heated to 100° C. and stirred for 16 h. LC-MS monitoring showed the reaction was completed. The reaction solution was cooled to room temperature and then poured slowly into 1000 mL of ice water. The aqueous phase was extracted with ethyl acetate (1000 mL×3), and the organic phase was dried over anhydrous sodium sulfate. The organic phase was concentrated under reduced pressure to give a crude product (31 g, yield: 73.9%), which was directly used in the next step.

¹H-NMR (400 MHz, DMSO-d6): δ=8.29 (d, 1H), 8.18 (d, 1H), 8.07 (d, 1H), 7.99 (d, 1H), 7.63 (dd, 1H).

### Step 2: Synthesis of Compound Int_88-A-3

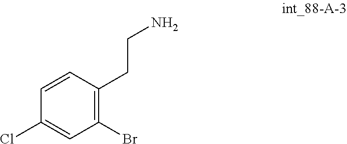 [SMILES: NCCc1ccc(Cl)cc1Br]

LiBH₄ (28.3 g, 1.3 mol) was dissolved in tetrahydrofuran (300 mL), and TMSCl (282 g, 2.6 mol, 329 mL) was added to the reaction solution at 0° C. under nitrogen atmosphere. The reaction solution was stirred at 0° C. for 30 min, and a solution of int_88-A-2 (34.1 g, 130 mmol) in tetrahydrofuran (250 mL) was slowly and dropwise added to the reaction solution within 30 min. The reaction solution was warmed to 75° C. and allowed to react for another 2 h. LC-MS monitoring showed the reaction was completed. The reaction solution was cooled to 0° C., and 600 mL of methanol was slowly added to quench the reaction. The organic phase was concentrated under reduced pressure to give a crude product, and the crude product was adjusted to pH>7 with ammonia water. The aqueous phase was extracted with ethyl acetate (500 mL×3), and the organic phase was dried over anhydrous sodium sulfate. The organic phase was concentrated under reduced pressure to give a crude product (23.5 g), which was directly used in the next step.

ESI-MS m/z: 234 [M+H]⁺.

### Step 3: Synthesis of Compound Int_88-A-4

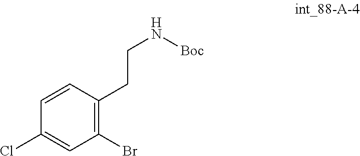 [SMILES: CC(C)(C)OC(=O)NCCc1ccc(Cl)cc1Br]

Int_88-A-3 (25 g, 106.6 mmol) and (Boc)₂O (46.5 g, 213.2 mmol) were dissolved in 1,4-dioxane (10 mL), and TEA (21.5 g, 213.2 mmol) was added to the reaction solution at room temperature. The reaction solution was warmed to 80° C. and allowed to react for 16 h. LC-MS monitoring showed the reaction was completed. After the reaction solution was cooled to room temperature, the reaction solution was concentrated under reduced pressure to give a crude product, and the crude product was diluted with water. The aqueous phase was extracted with ethyl acetate (500 mL×3), and the organic phase was dried over anhydrous sodium sulfate. The crude product was purified by preparative column chromatography to give a solid (27 g, yield: 75.8%).

ESI-MS m/z: 334 [M+H]⁺.

### Step 4: Synthesis of Compound Int_88-A-6

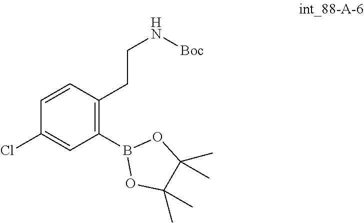 [SMILES: CC(C)(C)OC(=O)NCCc1ccc(Cl)cc1B1OC(C)(C)C(C)(C)O1]

Int_88-A-4 (5 g, 14.9 mmol), int_88-A-5 (7.56 g, 29.8 mmol), Pd(dppf)Cl₂ (1.09 g, 1.5 mmol), and potassium acetate (3.65 g, 37.25 mmol) were dissolved in 1,4-dioxane (150 mL). The reaction solution was purged with nitrogen three times and warmed to 100° C. and allowed to react for 6 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography to give a solid (3.9 g, yield: 68.6%).

ESI-MS m/z: 382 [M+H]⁺.

### Step 5: Synthesis of Compound Int_88-A-8

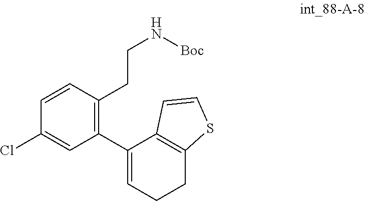 [SMILES: CC(C)(C)OC(=O)NCCc1ccc(Cl)cc1C1=CCCc2sccc21]

Int_88-A-6 (2 g, 5.23 mmol), int_88-A-7 (1.78 g, 6.28 mmol), Pd(dppf)Cl₂ (365.8 mg, 0.5 mmol), and potassium phosphate (2.22 g, 10.46 mmol) were dissolved in a mixed solvent of 1,4-dioxane (50 mL) and water (5 mL). The reaction solution was purged with nitrogen three times and warmed to 100° C. and allowed to react for 6 h. LC-MS monitoring showed the reaction was completed. The reaction solution was poured slowly into 100 mL of ice water. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (1.65 g, yield: 81.2%).

ESI-MS m/z: 390 [M+H]⁺.

### Step 6: Synthesis of Compound Int_88-9

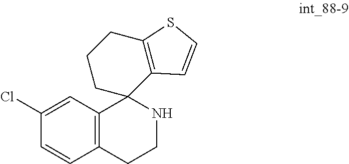 [SMILES: Clc1ccc2c(c1)C1(CCCc3sccc31)NCC2]

Int_88-A-8 (1 g, 2.56 mmol) was dissolved in dichloromethane (10 mL). TFA (10 mL) was added to the reaction solution under nitrogen atmosphere, and the reaction solution was allowed to react at room temperature for 8 h. LC-MS monitoring showed the reaction was completed. Saturated aqueous sodium bicarbonate solution (50 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (151 mg, yield: 20.3%).

¹H NMR (400 MHz, DMSO-d6) δ 7.14-7.11 (m, 3H), 6.68 (t, J=1.4 Hz, 1H), 6.35 (d, J=5.2 Hz, 1H), 2.97 (m, 2H), 2.88-2.79 (m, 3H), 2.71-2.62 (m, 1H), 2.16 (ddd, J=13.1, 5.0, 2.7 Hz, 1H), 2.03 (tdd, J=12.9, 6.9, 3.3 Hz, 1H), 1.84-1.72 (m, 2H).

ESI-MS m/z: 290 [M+H]⁺.

### Example 6. Synthesis of Compound 91

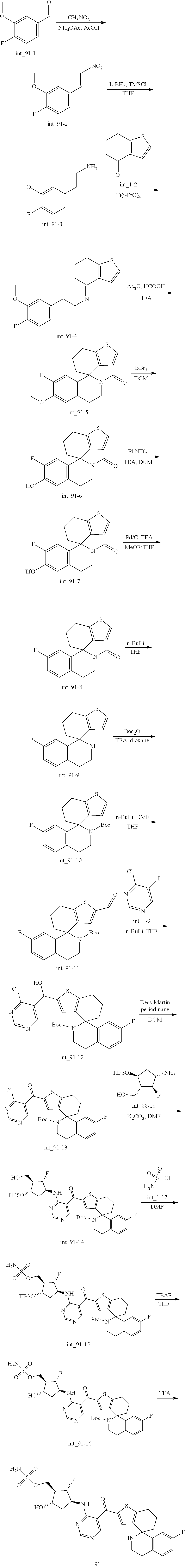

### Step 1: Synthesis of Compound Int_91-2

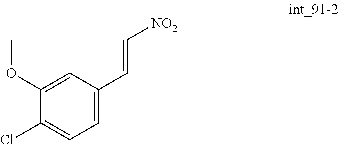

Int_91-1 (25.0 g, 162 mmol) and NH₄OAc (28.8 g, 373 mmol) were dissolved in acetic acid (250 mL), and the mixed solution was purged with nitrogen three times. CH₃NO₂ (25.7 g, 422 mmol, 22.8 mL) was added to the reaction solution under nitrogen atmosphere, and the reaction solution was heated to 100° C. and stirred for 16 h. LC-MS monitoring showed the reaction was completed. The reaction solution was cooled to room temperature and then poured slowly into 1000 mL of ice water. The aqueous phase was extracted with ethyl acetate (800 mL×3), and the organic phase was dried over anhydrous sodium sulfate. The organic phase was concentrated under reduced pressure to give a crude product (21 g, yield: 65.8%), which was directly used in the next step.

¹HNMR (400 MHz, DMSO-d6) δ=3.89 (s, 3H), 7.33 (dd, J=11.25, 8.44 Hz, 1H), 7.46 (ddd, J=8.25, 4.59, 1.96 Hz, 1H), 7.72 (dd, J=8.44, 1.96 Hz, 1H), 8.10 (d, J=13.57 Hz, 1H), 8.29 (d, J=13.57 Hz, 1H).

### Step 2: Synthesis of compound int_91-3

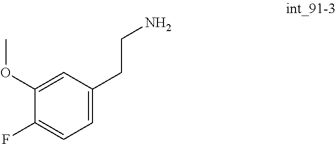

LiBH₄ (27.6 g, 1.27 mol) was dissolved in tetrahydrofuran (300 mL), and TMSCl (276 g, 2.54 mol, 322 mL) was added to the reaction solution at 0° C. under nitrogen atmosphere. The reaction solution was stirred at 0° C. for 30 min, and a solution of int_91-2 (25.0 g, 127 mmol) in tetrahydrofuran (250 mL) was slowly and dropwise added to the reaction solution within 30 min. The reaction solution was warmed to 75° C. and allowed to react for another 2 h. LC-MS monitoring showed the reaction was completed. The reaction solution was cooled to 0° C., and 600 mL of methanol was slowly added to quench the reaction. The organic phase was concentrated under reduced pressure to give a crude product, and the crude product was adjusted to pH>7 with ammonia water. The aqueous phase was extracted with ethyl acetate (500 mL×3), and the organic phase was dried over anhydrous sodium sulfate. The organic phase was concentrated under reduced pressure to give a crude product (25 g), which was directly used in the next step.

ESI-MS m/z: 170 [M+H]⁺.

### Step 3: Synthesis of Compound Int_91-4

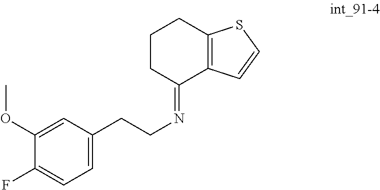 [SMILES: COc1cc(CC/N=C2\CCCc3sccc32)ccc1F]

Int_91-3 (12.5 g, 73.9 mmol) and int_1-2 (11.8 g, 77.6 mmol) were dissolved in Ti(i-PrO)₄ (1130 mL). The mixed solution was purged with nitrogen three times, heated to 80° C., and stirred for 2 h. LC-MS monitoring showed the reaction was completed. The reaction solution was cooled to room temperature, which was directly used in the next step.

ESI-MS m/z: 304 [M+H]⁺.

### Step 4: Synthesis of Compound Int_91-5

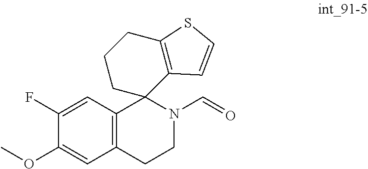 [SMILES: COc1cc2c(cc1F)C1(CCCc3sccc31)N(C=O)CC2]

HCOOH (400 mL) was slowly and dropwise added to Ac₂O (1000 mL) at −10° C., and after the addition was completed, the reaction solution was allowed to react at 20° C. for 0.5 h. Then, the int_91-4 solution obtained in step 3 was cooled to −10° C., and the mixed solution of HOOCH and Ac₂O described above was slowly and dropwise added to the int_91-4 solution. The temperature was kept at −10° C. during the dropwise addition. After the dropwise addition was completed, the reaction solution was warmed to 80° C. and allowed to react for 16 h. LC-MS monitoring showed the reaction was completed. The reaction solution was cooled to room temperature. The organic phase was concentrated under reduced pressure to give a crude product, and the crude product was adjusted to pH>7 with a saturated aqueous sodium bicarbonate solution. The aqueous phase was extracted with ethyl acetate (1000 mL×3), and the organic phase was dried over anhydrous sodium sulfate. The organic phase was concentrated under reduced pressure to give a crude product. The crude product was purified by preparative column chromatography (SiO₂, PE/EtOAc=3/2) to give a solid (1.5 g, yield: 5.05%).

¹HNMR (400 MHz, DMSO-d6) δ=1.52-1.66 (m, 1H), 1.82-1.94 (m, 2H), 2.28-2.38 (m, 1H), 2.78-2.86 (m, 2H), 2.89-2.97 (m, 2H), 3.10-3.22 (m, 1H), 3.82 (s, 3H), 4.40 (dt, J=12.93, 3.99 Hz, 1H), 6.22 (d, J=5.28 Hz, 1H), 6.52 (d, J=12.98 Hz, 1H), 6.97 (d, J=9.02 Hz, 1H), 7.30 (d, J=5.28 Hz, 1H), 7.57 (s, 1H).

ESI-MS m/z: 332 [M+H]⁺.

### Step 5: Synthesis of Compound Int_91-6

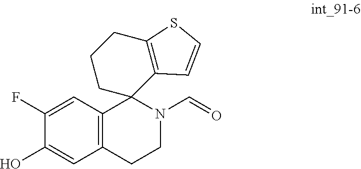 [SMILES: O=CN1CCc2cc(O)c(F)cc2C12CCCc1sccc12]

Int_91-5 (4.00 g, 12.1 mmol) was dissolved in dichloromethane (40 mL), and the mixed solution was purged with nitrogen three times. The reaction solution was cooled to 0° C., and BBr₃ (15.1 g, 60.4 mmol, 5.81 mL) was slowly and dropwise added to the reaction solution. The reaction solution was warmed to room temperature and allowed to react for 1 h. LC-MS monitoring showed the reaction was completed. The reaction solution was poured slowly into 200 mL of ice water. The aqueous phase was extracted with ethyl acetate (200 mL×3), and the organic phase was dried over anhydrous sodium sulfate. The organic phase was concentrated under reduced pressure to give a crude product (4.5 g, yield: 99.3%), which was directly used in the next step.

ESI-MS m/z: 318 [M+H]⁺.

### Step 6: Synthesis of Compound Int_91-7

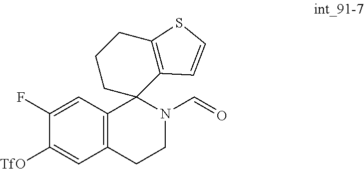 [SMILES: O=CN1CCc2cc(OS(=O)(=O)C(F)(F)F)c(F)cc2C12CCCc1sccc12]

Int_91-6 (4.50 g, 14.2 mmol), PhNTf₂ (9.12 g, 25.5 mmol), and TEA (3.59 g, 35.5 mmol, 4.93 mL) were dissolved in dichloromethane (50 mL) at 0° C. The reaction solution was purged with nitrogen three times, and allowed to react at room temperature for 16 h. LC-MS monitoring showed the reaction was completed. The reaction solution was poured slowly into 200 mL of ice water. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a solid (4 g, yield: 62.8%).

¹HNMR (400 MHz, DMSO-d6) δ=1.52-1.67 (m, 1H), 1.86-1.94 (m, 1H), 1.99 (s, 1H), 2.41 (dt, J=11.83, 2.01 Hz, 1H), 2.91 (br s, 2H), 2.94 (br dd, J=8.03, 3.85 Hz, 2H), 3.18 (ddd, J=13.20, 10.45, 4.95 Hz, 1H), 4.40-4.49 (m, 1H), 6.26 (d, J=5.28 Hz, 1H), 6.98 (d, J=11.66 Hz, 1H), 7.35 (d, J=5.28 Hz, 1H), 7.56 (s, 1H), 7.60 (d, J=7.70 Hz, 1H).

ESI-MS m/z: 450 [M+H]⁺.

### Step 7: Synthesis of Compound Int_91-8

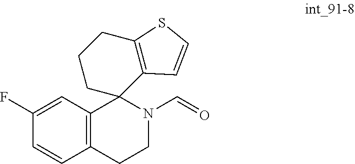 [SMILES: O=CN1CCc2ccc(F)cc2C12CCCc1sccc12]

Int_91-7 (4.00 g, 8.90 mmol), Pd/C (1.00 g, 10.0% purity), and TEA (3.60 g, 35.6 mmol, 4.96 mL) were dissolved in methanol (80 mL). The reaction solution was purged with hydrogen three times, and allowed to react at room temperature for 16 h under hydrogen atmosphere (20.0 Psi). LC-MS monitoring showed the reaction was completed. The reaction solution was filtered to give a filtrate, and the filtrate was concentrated under reduced pressure to give a crude product. The crude product was purified by preparative column chromatography (SiO₂, PE/EtOAc=1/1) to give a solid (2.2 g, yield: 81.2%).

¹HNMR (400 MHz, DMSO-d6) δ=1.53-1.67 (m, 1H), 1.84-1.93 (m, 1H), 1.98 (td, J=13.45, 2.69 Hz, 1H), 2.35-2.43 (m, 1H), 2.84 (br dd, J=7.27, 3.85 Hz, 2H), 2.94 (dd, J=8.01, 3.97 Hz, 2H), 3.19 (dt, J=12.93, 7.78 Hz, 1H), 4.40 (dt, J=13.02, 4.07 Hz, 1H), 6.22 (d, J=5.26 Hz, 1H), 6.53 (dd, J=10.51, 2.69 Hz, 1H), 7.04 (td, J=8.50, 2.69 Hz, 1H), 7.25 (dd, J=8.50, 6.05 Hz, 1H), 7.32 (d, J=5.26 Hz, 1H), 7.59 (s, 1H).

ESI-MS m/z: 302 [M+H]⁺.

### Step 8: Synthesis of Compound Int_91-9

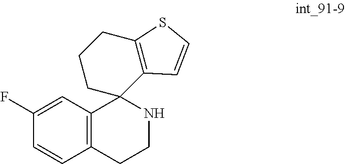 [SMILES: Fc1ccc2c(c1)C1(CCCc3sccc31)NCC2]

Int_91-8 (2.20 g, 7.30 mmol) was dissolved in THF (25 mL), and the mixture was cooled to −78° C. n-BuLi (2.50 M, 8.76 mL) was slowly and dropwise added to the reaction solution under nitrogen atmosphere, and the reaction solution was allowed to react at −30° C. for 2 h. Subsequently, the reaction solution was warmed to room temperature and allowed to react for 0.5 h. LC-MS monitoring showed the reaction was completed. A saturated aqueous ammonium chloride solution (50 mL) was added to the reaction solution. The aqueous phase was extracted with ethyl acetate (50 mL×3), and the organic phase was dried over anhydrous sodium sulfate.

The organic phase was concentrated under reduced pressure to give a crude product. The crude product was purified by preparative column chromatography (SiO₂, DCM/MeOH=95/5) to give a solid (1.4 g, yield: 56.1%).

¹HNMR (400 MHz, DMSO-d6) δ=1.72-1.84 (m, 2H), 1.96-2.08 (m, 1H), 2.10-2.22 (m, 1H), 2.67 (br s, 1H), 2.75-2.90 (m, 4H), 2.95-3.00 (m, 2H), 6.34 (d, J=5.28 Hz, 1H), 6.45 (dd, J=10.56, 2.64 Hz, 1H), 6.92 (td, J=8.53, 2.75 Hz, 1H), 7.10 (br s, 1H), 7.11-7.15 (m, 1H).

ESI-MS m/z: 274 [M+H]⁺.

### Step 9: Synthesis of Compound Int_91-10

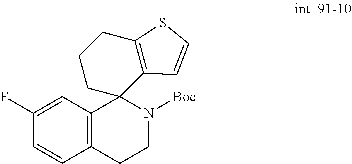 [SMILES: CC(C)(C)OC(=O)N1CCc2ccc(F)cc2C12CCCc1sccc12]

Int_91-9 (1.40 g, 5.12 mmol) and (Boc)₂O (2.46 g, 11.3 mmol, 2.59 mL) were dissolved in 1,4-dioxane (15 mL), and TEA (1.55 g, 15.4 mmol, 2.14 mL) was added to the reaction solution at room temperature. The reaction solution was warmed to 80° C. and allowed to react for 16 h. LC-MS monitoring showed the reaction was completed. After the reaction solution was cooled to room temperature, the reaction solution was concentrated under reduced pressure to give a crude product. The crude product was purified by preparative column chromatography (SiO₂, PE/EtOAc=4/1) to give a solid (870 mg, yield: 45.2%).

ESI-MS m/z: 374 [M+H]⁺.

### Step 10: Synthesis of Compound Int_91-11

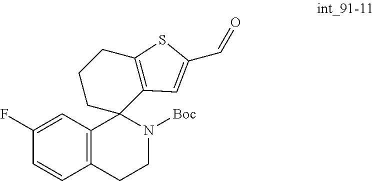 [SMILES: CC(C)(C)OC(=O)N1CCc2ccc(F)cc2C12CCCc1sc(C=O)cc12]

Int_91-10 (900 mg, 2.41 mmol) was dissolved in THF (10 mL), and the mixture was cooled to −70° C. n-BuLi (2.50 M, 2.89 mL) was slowly and dropwise added to the reaction solution under nitrogen atmosphere, and the reaction solution was allowed to react at −70° C. for 0.5 h. Then, DMF (528 mg, 7.23 mmol, 556 μL) was dropwise added to the reaction solution at −70° C., and after the dropwise addition was completed, the reaction solution was allowed to react at −75° C. for 1 h. LC-MS monitoring showed the reaction was completed. A saturated ammonium chloride solution (25 mL) was slowly added to the reaction solution. The aqueous phase was extracted with ethyl acetate (30 mL×3), and the organic phase was dried over anhydrous sodium sulfate.

The organic phase was concentrated under reduced pressure to give a crude product. The crude product was purified by preparative column chromatography (SiO₂, PE/EtOAc=4/1) to give a solid (970 mg, yield: 95.9%).

¹HNMR (400 MHz, CHLOROFORM-d) δ=1.23 (s, 9H), 1.66-1.80 (m, 2H), 1.82-1.91 (m, 1H), 2.73-2.94 (m, 3H), 2.95-3.06 (m, 2H), 3.25 (td, J=12.07, 3.12 Hz, 1H), 4.40-4.49 (m, 1H), 6.25 (dd, J=10.45, 2.63 Hz, 1H), 6.90 (td, J=8.25, 2.57 Hz, 1H), 7.17 (dd, J=8.07, 5.87 Hz, 1H), 7.27-7.28 (m, 1H), 9.75 (s, 1H).

MS (ESI): 402 [M+H]⁺.

### Step 11: Synthesis of Compound Int_91-12

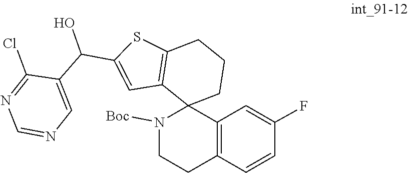 [SMILES: CC(C)(C)OC(=O)N1CCc2ccc(F)cc2C12CCCc1sc(C(O)c3cncnc3Cl)cc12]

Int_1-9 (1.62 g, 6.72 mmol) was dissolved in THF (15 mL), and the mixture was cooled to −75° C. n-BuLi (2.50 M, 5.38 mL) was slowly and dropwise added to the reaction solution under nitrogen atmosphere, and the reaction solution was allowed to react at −75° C. for 1 h. Then, a solution of int_91-11 (900 mg, 2.24 mmol) in THF (25 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (100 mL) was slowly added to the reaction solution. The aqueous phase was extracted with ethyl acetate (100 mL×3), and the organic phase was dried over anhydrous sodium sulfate.

The organic phase was concentrated under reduced pressure to give a crude product. The crude product was purified by preparative column chromatography (SiO₂, PE/EtOAc=7/3) to give a solid (690 mg, yield: 59.7%).

¹HNMR (400 MHz, DMSO-d6) δ=0.94-1.18 (m, 9H), 1.44-1.80 (m, 4H), 2.55-2.71 (m, 2H), 2.77-2.94 (m, 3H), 3.01-3.12 (m, 1H), 4.18-4.28 (m, 1H), 5.96-6.12 (m, 1H), 6.16-6.27 (m, 1H), 6.40-6.45 (m, 1H), 6.57-6.72 (m, 1H), 6.99-7.07 (m, 1H), 7.28 (br dd, J=8.47, 6.05 Hz, 1H), 8.93-8.97 (m, 1H).

MS (ESI): 516 [M+H]⁺.

### Step 12: Synthesis of Compound Int_91-13

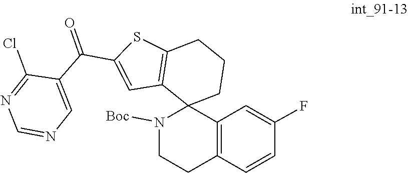 [SMILES: CC(C)(C)OC(=O)N1CCc2ccc(F)cc2C12CCCc1sc(C(=O)c3cncnc3Cl)cc12]

Int_91-12 (640 mg, 1.24 mmol) was dissolved in DCM (10 mL), and a Dess-Martin oxidant (1.05 g, 2.48 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography (SiO₂, PE/EtOAc=10/1) to give a solid (630 mg, yield: 98.8%).

¹HNMR (400 MHz, DMSO-d6) δ=1.19 (s, 9H), 1.54-1.66 (m, 2H), 1.75-1.85 (m, 1H), 2.59-2.81 (m, 2H), 2.85-2.91 (m, 2H), 3.03-3.18 (m, 2H), 4.17-4.26 (m, 1H), 6.22 (dd, J=10.78, 2.64 Hz, 1H), 7.05 (td, J=8.47, 2.64 Hz, 1H), 7.25-7.34 (m, 2H), 8.86 (s, 1H), 9.15 (s, 1H).

MS (ESI): 514 [M+H]⁺.

### Step 13: Synthesis of Compound Int_91-14

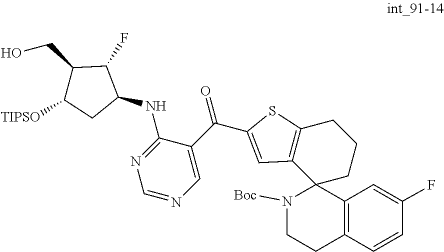

Int_91-13 (390 mg, 759 μmol) and int_88-18 (348 mg, 1.14 mmol) were dissolved in DMF (10 mL), and K₂CO₃ (524 mg, 3.79 mmol) was added to the reaction solution at room temperature. The reaction solution was allowed to react at room temperature for 16 h. LC-MS monitoring showed the reaction was completed. Ice water (50 mL) was added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography (SiO₂, PE/EtOAc=7/3) to give a product (378 mg, yield: 63.6%).

MS (ESI): 783 [M+H]⁺.

### Step 14: Synthesis of Compound Int_91-15

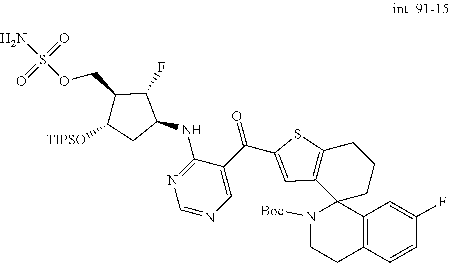

Int_91-14 (563 mg, 719 μmol) was dissolved in DMF (10 mL), and int_1-17 (249 mg, 2.16 mmol) was added to the reaction solution at room temperature. The reaction solution was allowed to react at room temperature for 0.5 h. LC-MS monitoring showed the reaction was completed. Ice water (20 mL) was added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography to give a solid (605 mg, yield: 97.7%).

MS (ESI): 862 [M+H]⁺.

### Step 15: Synthesis of Compound Int_91-16

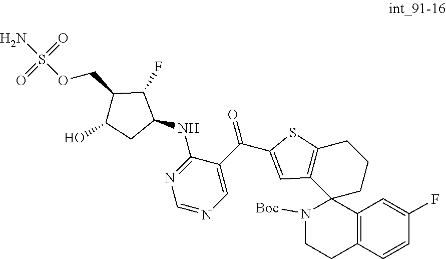 [SMILES: CC(C)(C)OC(=O)N1CCc2ccc(F)cc2C12CCCc1sc(C(=O)c3cncnc3N[C@H]3C[C@H](O)[C@@H](COS(N)(=O)=O)[C@@H]3F)cc12]

Int_91-15 (562 mg, 652 μmol) was dissolved in THF (10 mL), and TBAF (1.00 M, 652 μL) was added to the reaction solution at room temperature. The reaction solution was allowed to react at room temperature for 16 h. LC-MS monitoring showed the reaction was completed. Water (20 mL) was added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography (SiO₂, PE/EtOAc=4/1) to give a solid (412 mg, yield: 89.5%).

MS (ESI): 706 [M+H]⁺.

### Step 16: Synthesis of Compound 91

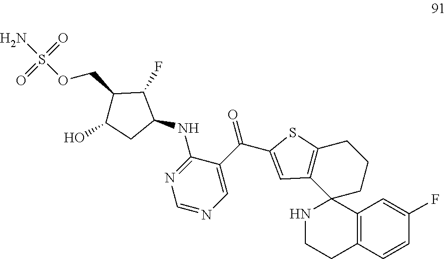 [SMILES: NS(=O)(=O)OC[C@H]1[C@H](F)[C@@H](Nc2ncncc2C(=O)c2cc3c(s2)CCCC32NCCc3ccc(F)cc32)C[C@@H]1O]

Int_91-16 (200 mg, 283 μmol) was dissolved in DCM (5 mL) at room temperature, and the mixture was added into TFA (2 mL). The reaction solution was allowed to react at room temperature for 5 min. LC-MS monitoring showed the reaction was completed. The reaction solution was cooled to 0° C. A saturated aqueous sodium bicarbonate solution was added to the reaction solution to adjust the pH to 8. The aqueous phase was extracted with ethyl acetate (20 mL×3), and the organic phase was dried over anhydrous sodium sulfate. The organic phase was concentrated under reduced pressure to give a crude product. The crude product was purified by preparative HPLC to give a solid (60 mg, yield: 35.0%).

MS (ESI): 606 [M+H]⁺.

### Example 7. Synthesis of Compound 92 and Compound 93

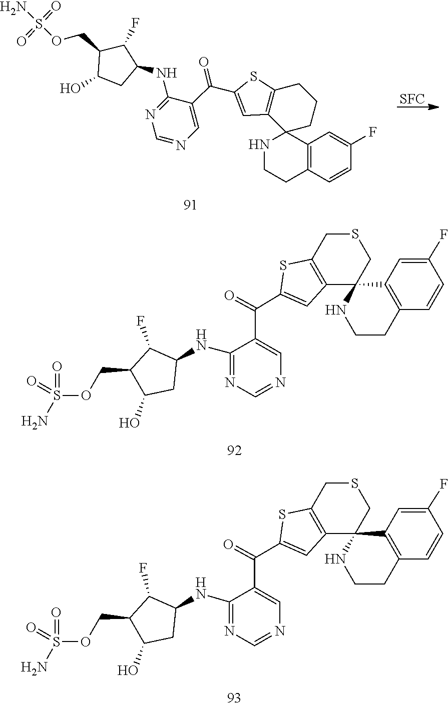 [SMILES: NS(=O)(=O)OC[C@H]1[C@H](F)[C@@H](Nc2ncncc2C(=O)c2cc3c(s2)CCCC32NCCc3ccc(F)cc32)C[C@@H]1O ; "SFC" ; NS(=O)(=O)OC[C@H]1[C@H](F)[C@@H](Nc2ncncc2C(=O)c2cc3c(s2)CSC[C@@]32NCCc3ccc(F)cc32)C[C@@H]1O ; NS(=O)(=O)OC[C@H]1[C@H](F)[C@@H](Nc2ncncc2C(=O)c2cc3c(s2)CSC[C@]32NCCc3ccc(F)cc32)C[C@@H]1O]

Compound 91 (100 mg, 0.165 mmol) was subjected to SFC chiral resolution (column: DAICEL CHIRALPAK IG (250 mm×30 mm, 10 μm); mobile phase: [CO₂-iso-propanol (0.1% NH₃H₂O)]; B %: 50%, isocratic elution mode) to give compound 92 (35 mg) and compound 93 (37 mg).

Compound 92: ¹H NMR (400 MHz, DMSO-d6) δ 8.57 (s, 1H), 8.49 (s, 1H), 8.38 (d, J=8.2 Hz, 1H), 7.53 (s, 2H), 7.18-7.07 (m, 1H), 6.94 (d, J=4.2 Hz, 2H), 6.56 (dd, J=10.3, 2.8 Hz, 1H), 5.19 (d, J=4.6 Hz, 1H), 5.12-4.97 (m, 1H), 4.89 (dt, J=54.1, 6.5 Hz, 1H), 4.27-4.04 (m, 2H), 3.93 (s, 1H), 2.98 (m, 4H), 2.84 (d, J=7.7 Hz, 1H), 2.65 (m, 1H), 2.35-2.13 (m, 2H), 2.08 (m, 1H), 1.98 (m, 1H), 1.83 (d, J=13.2 Hz, 3H); MS (ESI): 606 [M+H]⁺.

Analytical SFC retention time: 2.764 min (Instrument: Waters UPCC with PDA Detector; Column: Chiralpak IG-3 100×4.6 mm I.D., 3 μm; Mobile phase: A: CO₂, B: iso-propanol (0.05% DEA); Isocratic: 40% B; Flow rate: 2.8 mL/min; Column temp: 35° C.; ABPR: 1500 psi).

Compound 93: ¹H NMR (400 MHz, DMSO-d6) δ 8.57 (s, 1H), 8.48 (s, 1H), 8.33 (d, J=8.2 Hz, 1H), 7.53 (s, 2H), 7.19-7.06 (m, 1H), 7.02-6.88 (m, 2H), 6.55 (dd, J=10.3, 2.8 Hz, 1H), 5.18 (d, J=4.6 Hz, 1H), 5.10-5.00 (m, 1H), 4.88 (dt, J=54.2, 6.8 Hz, 1H), 4.26-4.04 (m, 2H), 3.91 (s, 1H), 2.97 (m, 4H), 2.86 (dd, J=16.0, 7.8 Hz, 1H), 2.65 (m, 1H), 2.35-2.14 (m, 2H), 2.08 (s, 1H), 1.95 (s, 1H), 1.81 (t, J=11.8 Hz, 3H); MS (ESI): 606 [M+H]⁺.

Analytical SFC retention time: 1.291 min (Instrument: Waters UPCC with PDA Detector; Column: Chiralpak IG-3 100x4.6 mm I.D., 3 um; Mobile phase: A: CO₂, B: iso-propanol (0.05% DEA); Isocratic: 40% B; Flow rate: 2.8 mL/min; Column temp: 35° C.; ABPR: 1500 psi).

### Example 8. Synthesis of Intermediate int 91-9

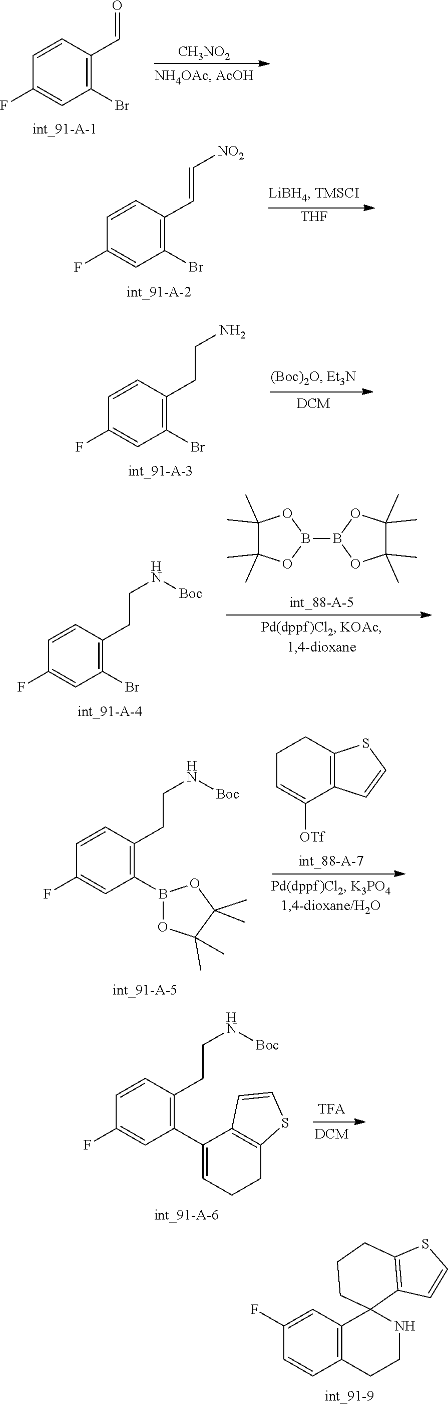

### Step 1: Synthesis of Compound Int_91-A-2

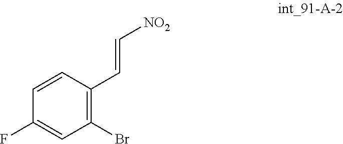 [SMILES: O=[N+]([O-])/C=C/c1ccc(F)cc1Br]

Int_91-A-1 (32.4 g, 160 mmol) and NH₄OAc (28.8 g, 373 mmol) were dissolved in acetic acid (250 mL), and the mixed solution was purged with nitrogen three times. CH₃NO₂ (25.7 g, 422 mmol, 22.8 mL) was added to the reaction solution under nitrogen atmosphere, and the reaction solution was heated to 100° C. and stirred for 16 h. LC-MS monitoring showed the reaction was completed. The reaction solution was cooled to room temperature and then poured slowly into 1000 mL of ice water. The aqueous phase was extracted with ethyl acetate (1000 mL×3), and the organic phase was dried over anhydrous sodium sulfate. The organic phase was concentrated under reduced pressure to give a crude product (32.3 g, yield: 82.1%), which was directly used in the next step.

### Step 2: Synthesis of Compound Int_91-A-3

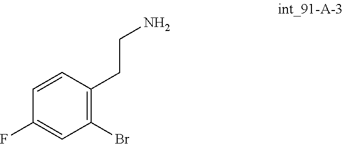 [SMILES: NCCc1ccc(F)cc1Br]

LiBH₄ (28.3 g, 1.3 mol) was dissolved in tetrahydrofuran (300 mL), and TMSCl (282 g, 2.6 mol, 329 mL) was added to the reaction solution at 0° C. under nitrogen atmosphere. The reaction solution was stirred at 0° C. for 30 min, and a solution of int_91-A-2 (31.9 g, 130 mmol) in tetrahydrofuran (250 mL) was slowly and dropwise added to the reaction solution within 30 min. The reaction solution was warmed to 75° C. and allowed to react for another 2 h. LC-MS monitoring showed the reaction was completed. The reaction solution was cooled to 0° C., and 600 mL of methanol was slowly added to quench the reaction. The organic phase was concentrated under reduced pressure to give a crude product, and the crude product was adjusted to pH>7 with ammonia water. The aqueous phase was extracted with ethyl acetate (500 mL×3), and the organic phase was dried over anhydrous sodium sulfate. The organic phase was concentrated under reduced pressure to give a crude product (20.7 g), which was directly used in the next step.

ESI-MS m/z: 218 [M+H]⁺.

### Step 3: Synthesis of Compound Int_91-A-4

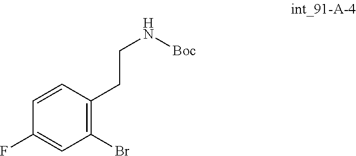 [SMILES: CC(C)(C)OC(=O)NCCc1ccc(F)cc1Br]

Int_91-A-3 (23.2 g, 106.6 mmol) and (Boc)₂O (46.5 g, 213.2 mmol) were dissolved in 1,4-dioxane (10 mL), and TEA (21.5 g, 213.2 mmol) was added to the reaction solution at room temperature. The reaction solution was warmed to 80° C. and allowed to react for 16 h. LC-MS monitoring showed the reaction was completed. After the reaction solution was cooled to room temperature, the reaction solution was concentrated under reduced pressure to give a crude product, and the crude product was diluted with water. The aqueous phase was extracted with ethyl acetate (500 mL×3), and the organic phase was dried over anhydrous sodium sulfate. The crude product was purified by preparative column chromatography to give a solid (28.1 g, yield: 82.8%).

ESI-MS m/z: 318 [M+H]⁺.

### Step 4: Synthesis of Compound Int_91-A-5

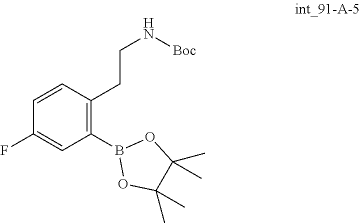 [SMILES: CC(C)(C)OC(=O)NCCc1ccc(F)cc1B1OC(C)(C)C(C)(C)O1]

Int_91-A-4 (4.7 g, 14.9 mmol), int_88-A-5 (7.56 g, 29.8 mmol), Pd(dppf)Cl₂ (1.09 g, 1.5 mmol), and potassium acetate (3.65 g, 37.25 mmol) were dissolved in 1,4-dioxane (150 mL). The reaction solution was purged with nitrogen three times and warmed to 100° C. and allowed to react for 6 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography to give a solid (3.7 g, yield: 68.5%).

ESI-MS m/z: 366 [M+H]⁺.

### Step 5: Synthesis of Compound Int_91-A-6

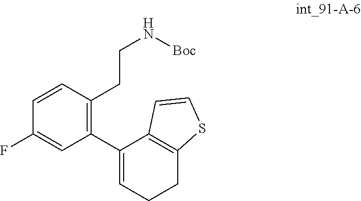 [SMILES: CC(C)(C)OC(=O)NCCc1ccc(F)cc1C1=CCCc2sccc21]

Int_91-A-5 (1.9 g, 5.23 mmol), int_88-A-7 (1.78 g, 6.28 mmol), Pd(dppf)Cl₂ (365.8 mg, 0.5 mmol), and potassium phosphate (2.22 g, 10.46 mmol) were dissolved in a mixed solvent of 1,4-dioxane (50 mL) and water (5 mL). The reaction solution was purged with nitrogen three times and warmed to 100° C. and allowed to react for 6 h. LC-MS monitoring showed the reaction was completed. The reaction solution was poured slowly into 100 mL of ice water. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (1.37 g, yield: 70.2%).

ESI-MS m/z: 374 [M+H]⁺.

### Step 6: Synthesis of Compound Int_91-9

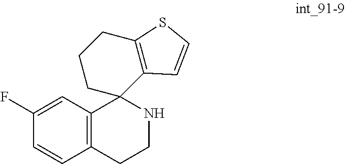 [SMILES: Fc1ccc2c(c1)C1(CCCc3sccc31)NCC2]

Int_91-A-6 (1.1 g, 3 mmol) was dissolved in dichloromethane (10 mL). TFA (10 mL) was added to the reaction solution under nitrogen atmosphere, and the reaction solution was allowed to react at room temperature for 8 h. LC-MS monitoring showed the reaction was completed. Saturated aqueous sodium bicarbonate solution (50 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (125 mg, yield: 15.2%).

¹HNMR (400 MHz, DMSO-d6) δ=1.72-1.84 (m, 2H), 1.96-2.08 (m, 1H), 2.10-2.22 (m, 1H), 2.67 (br s, 1H), 2.75-2.90 (m, 4H), 2.95-3.00 (m, 2H), 6.34 (d, J=5.28 Hz, 1H), 6.45 (dd, J=10.56, 2.64 Hz, 1H), 6.92 (td, J=8.53, 2.75 Hz, 1H), 7.10 (br s, 1H), 7.11-7.15 (m, 1H).

ESI-MS m/z: 274 [M+H]⁺.

### Example 9. Synthesis of Compound 103

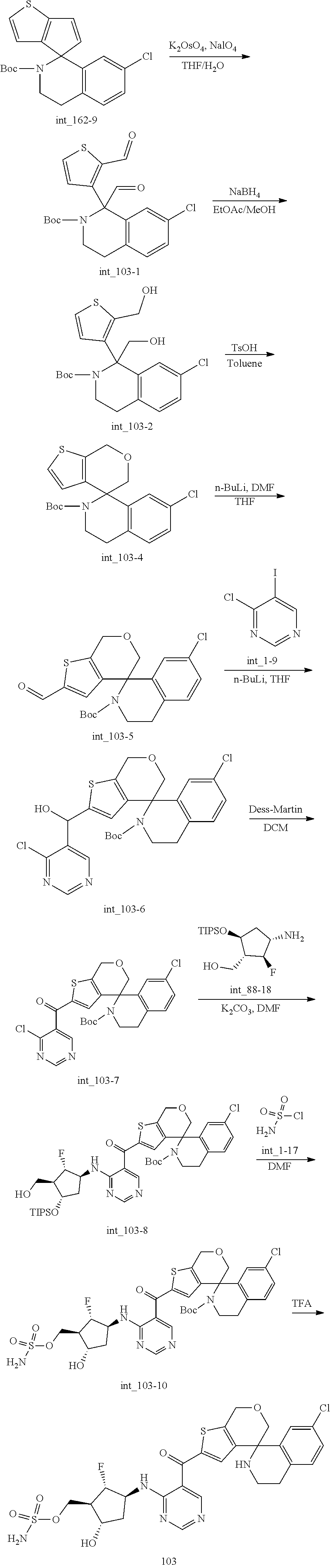

### Step 1: Synthesis of Compound Int_103-1

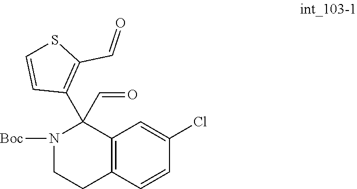 [SMILES: CC(C)(C)OC(=O)N1CCc2ccc(Cl)cc2C1(C=O)c1ccsc1C=O]

Int_162-9 (2.5 g, 6.686 mmol), sodium periodate (7.2 g, 33.431 mmol) and K₂OsO₄·2H₂O (250 mg, 0.678 mmol) were dissolved in a mixed solvent of tetrahydrofuran (250 mL) and water (125 mL). The reaction solution was purged with nitrogen three times, and allowed to react at room temperature for 2 h. LC-MS monitoring showed the reaction was completed. The reaction solution was poured slowly into 100 mL of ice water. The aqueous phase was extracted with dichloromethane (100 mL×3), and the organic phase was dried over anhydrous sodium sulfate. The organic phase was concentrated under reduced pressure to give a crude product. The crude product was purified by preparative column chromatography to give a solid (2.6 g, yield: 96.2%).

ESI-MS m/z: 406 [M+H]⁺.

### Step 2: Synthesis of Compound Int_103-2

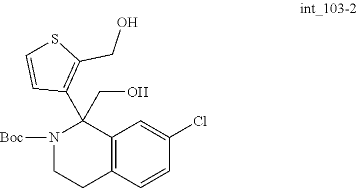 [SMILES: CC(C)(C)OC(=O)N1CCc2ccc(Cl)cc2C1(CO)c1ccsc1CO]

Int_103-1 (2.7 g, 6.686 mmol) was dissolved in a mixed solvent of ethyl acetate (60 mL) and methanol (15 mL), and sodium borohydride (1.0 g, 26.744 mmol) was added to the reaction solution. The reaction solution was allowed to react at room temperature for 2 h. LC-MS monitoring showed the reaction was completed. The reaction solution was poured slowly into 100 mL of ice water. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a solid (1.8 g, yield: 65.6%).

ESI-MS m/z: 410 [M+H]⁺.

### Step 3: Synthesis of Compound Int_103-3

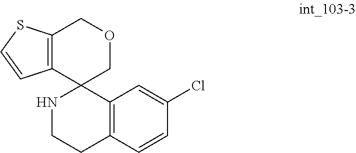 [SMILES: Clc1ccc2c(c1)C1(COCc3sccc31)NCC2]

Int_103-2 (1.2 g, 2.93 mmol) was dissolved in methylbenzene (50 mL), and p-toluenesulfonic acid monohydrate (5.6 g, 29.3 mmol) was added to the reaction solution. The reaction solution was warmed to 110° C. and allowed to react for 1 h. LC-MS monitoring showed the reaction was completed. The reaction solution was cooled to room temperature and then poured slowly into 50 mL of ice water. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (310 mg, yield: 38.7%).

ESI-MS m/z: 292 [M+H]⁺.

### Step 4: Synthesis of Compound Int_103-4

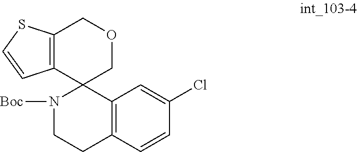 [SMILES: CC(C)(C)OC(=O)N1CCc2ccc(Cl)cc2C12COCc1sccc12]

Int_103-3 (1.87 g, 6.426 mmol) and (Boc)₂O (2.1 g, 9.639 mmol) were dissolved in 1,4-dioxane (16 mL), and TEA (973 mg, 9.639 mmol) was added to the reaction solution at room temperature. The reaction solution was warmed to 80° C. and allowed to react for 16 h under nitrogen atmosphere. LC-MS monitoring showed the reaction was completed. The reaction solution was cooled to room temperature and adjusted to a weakly acidic pH with 2 M aqueous hydrochloric acid solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a solid (1.6 g, yield: 63.5%).

ESI-MS m/z: 392 [M+H]⁺.

### Step 5: Synthesis of Compound Int_103-5

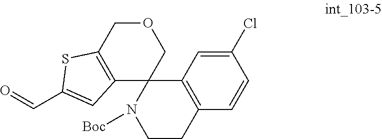 [SMILES: CC(C)(C)OC(=O)N1CCc2ccc(Cl)cc2C12COCc1sc(C=O)cc12]

Int_103-4 (265 mg, 0.676 mmol) was dissolved in THF (10 mL), and the mixture was cooled to −70° C. n-BuLi (2.5 M, 0.81 mL) was slowly and dropwise added to the reaction solution under nitrogen atmosphere, and the reaction solution was allowed to react at −70° C. for 1 h. Then, DMF (148 mg, 2.028 mmol) was dropwise added to the reaction solution at −60° C., and after the dropwise addition was completed, the reaction solution was allowed to react at 70° C. for 1 h. LC-MS monitoring showed the reaction was completed. A saturated ammonium chloride solution (20 mL) was slowly added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography (SiO₂, PE/EtOAc=5/1) to give a solid (170 mg, yield: 60.1%).

MS (ESI): 420 [M+H]⁺.

### Step 6: Synthesis of Compound Int_103-6

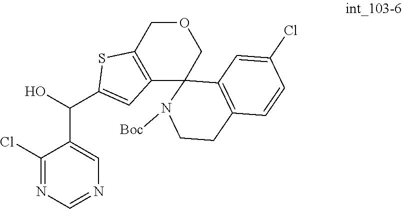 [SMILES: CC(C)(C)OC(=O)N1CCc2ccc(Cl)cc2C12COCc1sc(C(O)c3cncnc3Cl)cc12]

Int_1-9 (592 mg, 2.463 mmol) was dissolved in THF (10 mL), and the mixture was cooled to −70° C. n-BuLi (2.5 M, 2 mL, 4.926 mmol) was slowly and dropwise added to the reaction solution under nitrogen atmosphere, and the reaction solution was allowed to react at −70° C. for 1 h. Then, a solution of int_103-5 (170 mg, 0.411 mmol) in THF (1 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (20 mL) was slowly added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography to give a solid (180 mg, yield: 81.2%).

MS (ESI): 534 [M+H]⁺.

### Step 7: Synthesis of Compound Int_103-7

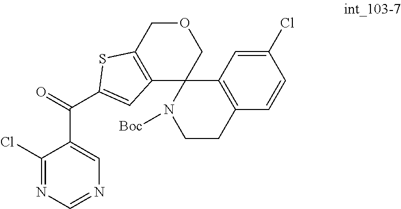 [SMILES: CC(C)(C)OC(=O)N1CCc2ccc(Cl)cc2C12COCc1sc(C(=O)c3cncnc3Cl)cc12]

Int_103-6 (180 mg, 0.337 mmol) was dissolved in DCM (15 mL), and a Dess-Martin oxidant (172 mg, 0.404 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a solid (156 mg, yield: 87.1%).

MS (ESI): 532 [M+H]⁺.

### Step 8: Synthesis of Compound Int_103-8

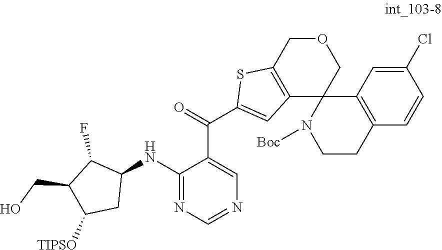

Int_103-7 (174 mg, 0.328 mmol) and int_88-18 (150 mg, 0.491 mmol) were dissolved in DMF (10 mL), and K₂CO₃ (138 mg, 1 mmol) was added to the reaction solution at room temperature. The reaction solution was allowed to react at room temperature for 16 h. LC-MS monitoring showed the reaction was completed. Ice water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (SiO₂, PE/EtOAc=2/1) to give a product (235 mg, yield: 89.6%).

MS (ESI): 801 [M+H]⁺.

### Step 9: Synthesis of Compound Int_103-9

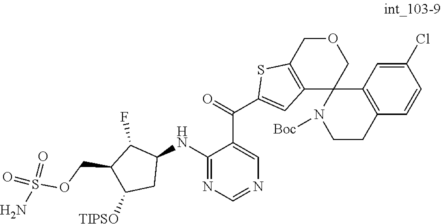

Int_103-8 (235 mg, 0.294 mmol) was dissolved in DMF (4 mL), and int_1-17 (67.8 mg, 0.587 mmol) was added to the reaction solution at room temperature. The reaction solution was allowed to react at room temperature for 1 h. LC-MS monitoring showed the reaction was completed. Ice water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a solid (230 mg, yield: 89.1%).

MS (ESI): 880 [M+H]⁺.

### Step 10: Synthesis of Compound Int_103-10

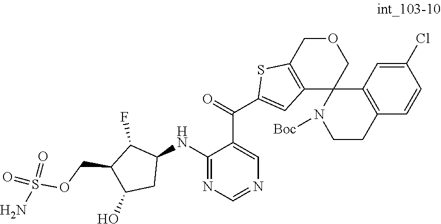 [SMILES: CC(C)(C)OC(=O)N1CCc2ccc(Cl)cc2C12COCc1sc(C(=O)c3cncnc3N[C@H]3C[C@H](O)[C@@H](COS(N)(=O)=O)[C@@H]3F)cc12]

Int_103-9 (253 mg, 0.288 mmol) was dissolved in THF (4 mL), and TBAF (1 M, 0.58 mL, 0.575 mmol) was added to the reaction solution at room temperature. The reaction solution was allowed to react at room temperature for 16 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a solid (175 mg, yield: 84.1%).

MS (ESI): 724 [M+H]⁺.

### Step 11: Synthesis of Compound 103

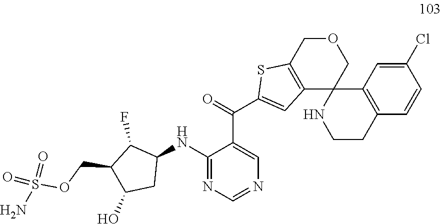 [SMILES: NS(=O)(=O)OC[C@H]1[C@H](F)[C@@H](Nc2ncncc2C(=O)c2cc3c(s2)COCC32NCCc3ccc(Cl)cc32)C[C@@H]1O]

Int_103-10 (176 mg, 0.244 mmol) was dissolved in dichloromethane (6 mL), and TFA (1.5 mL) was added to the reaction solution at room temperature. The reaction solution was allowed to react at room temperature for 10 min. LC-MS monitoring showed the reaction was completed. Ammonia water (1 mL) and water (10 mL) were added to the reaction solution. The aqueous phase was extracted with dichloromethane (10 mL×3), and the organic phase was dried over anhydrous sodium sulfate. The organic phase was concentrated under reduced pressure to give a crude product. The crude product was purified by preparative HPLC to give a solid (87 mg, yield: 57.2%).

MS (ESI): 624 [M+H]⁺.

### Example 10. Synthesis of Compound 104 and Compound 105

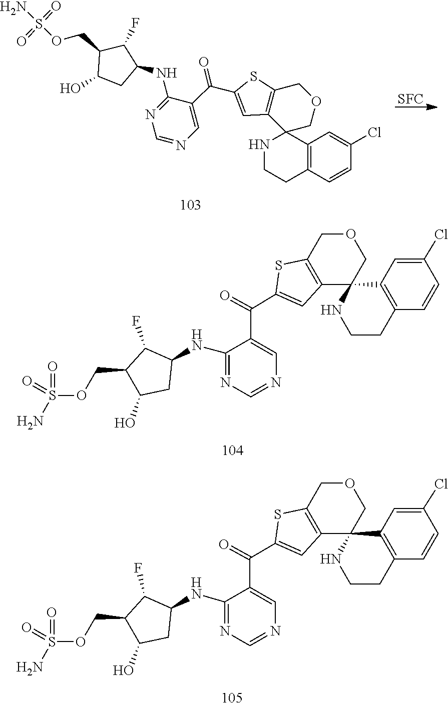 [SMILES: NS(=O)(=O)OC[C@H]1[C@H](F)[C@@H](Nc2ncncc2C(=O)c2cc3c(s2)COCC32NCCc3ccc(Cl)cc32)C[C@@H]1O ; "SFC" ; NS(=O)(=O)OC[C@H]1[C@H](F)[C@@H](Nc2ncncc2C(=O)c2cc3c(s2)COC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)COC[C@]32NCCc3ccc(Cl)cc32)C[C@@H]1O]

Compound 103 (80 mg, 0.128 mmol) was subjected to SFC chiral resolution (column: DAICEL CHIRALPAK IG (250 mm×30 mm, 10 m); mobile phase: [CO₂-EtOH (0.1% NH₃H₂O)]; B %: 50%, isocratic elution mode) to give compound 104 (21 mg) and compound 105 (15 mg).

Compound 104: ¹H NMR (400 MHz, DMSO-d6) δ 8.60 (s, 1H), 8.50 (s, 1H), 8.40 (d, J=8.2 Hz, 1H), 7.54 (s, 2H), 7.21 (d, J=8.8 Hz, 3H), 6.93 (s, 1H), 5.20 (d, J=4.6 Hz, 1H), 5.15-4.79 (m, 4H), 4.13 (qd, J=10.1, 6.0 Hz, 3H), 4.00-3.78 (m, 2H), 3.06 (s, 2H), 2.90 (s, 1H), 2.73 (s, 1H), 2.25 (d, J=22.4 Hz, 2H), 1.98 (s, 1H), 1.92-1.78 (m, 1H); MS (ESI): 624 [M+H]⁺.

Analytical SFC retention time: 3.492 min (Instrument: Waters UPCC with PDA Detector; Column: Chiralpak IG-3 100×4.6 mm I.D., 3 μm; Mobile phase: A: CO₂, B: Ethanol (0.05% DEA); Isocratic: 40% B; Flow rate: 2.5 mL/min; Column temp: 40° C.; ABPR: 100 bar).

Compound 105: ¹H NMR (400 MHz, DMSO-d6) δ 8.59 (s, 1H), 8.48 (s, 1H), 8.33 (d, J=8.3 Hz, 1H), 7.54 (s, 2H), 7.29-7.11 (m, 3H), 6.90 (d, J=2.0 Hz, 1H), 5.19 (d, J=4.6 Hz, 1H), 5.12-4.78 (m, 4H), 4.19-4.06 (m, 2H), 4.02 (d, J=11.4 Hz, 1H), 3.91 (s, 1H), 3.84 (d, J=11.4 Hz, 1H), 3.03 (s, 2H), 2.89 (dt, J=15.5, 7.4 Hz, 1H), 2.68 (d, J=16.5 Hz, 1H), 2.47-2.18 (m, 2H), 1.92 (d, J=9.3 Hz, 1H), 1.88-1.74 (m, 1H); MS (ESI): 624 [M+H]⁺.

Analytical SFC retention time: 2.146 min (Instrument: Waters UPCC with PDA Detector; Column: Chiralpak IG-3 100x4.6 mm I.D., 3 um; Mobile phase: A: CO₂, B: Ethanol (0.05% DEA); Isocratic: 40% B; Flow rate: 2.5 mL/min; Column temp: 40° C.; ABPR: 100bar).

### Example 11. Synthesis of Compound 130

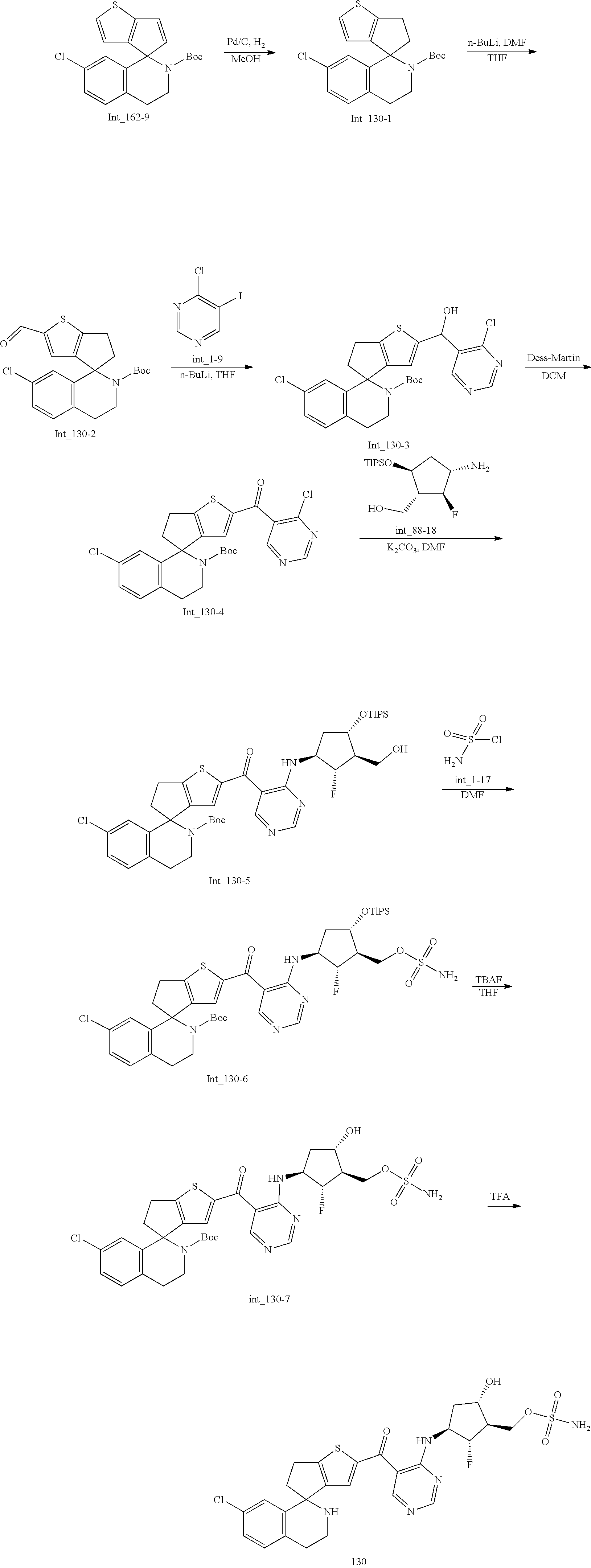

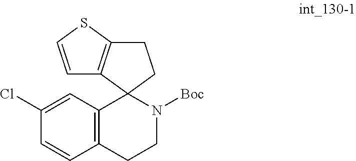 [SMILES: CC(C)(C)OC(=O)N1CCc2ccc(Cl)cc2C12CCc1sccc12]

### Step 2: Synthesis of Compound Int_130-2

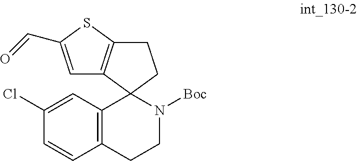 [SMILES: CC(C)(C)OC(=O)N1CCc2ccc(Cl)cc2C12CCc1sc(C=O)cc12]

Int_130-1 (0.30 g, 798 μmol) was dissolved in THF (10 mL), and the mixture was cooled to −60° C. n-BuLi (2.50 M, 479 μL) was slowly and dropwise added to the reaction solution under nitrogen atmosphere, and the reaction solution was allowed to react at −60° C. for 1 h. Then, DMF (175 mg, 2.39 mmol, 184 μL) was dropwise added to the reaction solution at −60° C., and after the dropwise addition was completed, the reaction solution was allowed to react at −60° C. for 3 h. LC-MS monitoring showed the reaction was completed. A saturated ammonium chloride solution (100 mL) was slowly added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (150 mg, yield: 46.5%).

MS (ESI): 404 [M+H]⁺.

### Step 3: Synthesis of Compound Int_130-3

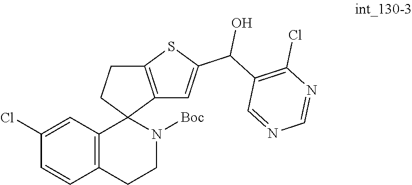 [SMILES: CC(C)(C)OC(=O)N1CCc2ccc(Cl)cc2C12CCc1sc(C(O)c3cncnc3Cl)cc12]

Int_1-9 (4.79 g, 19.9 mmol) was dissolved in THF (50 mL), and the mixture was cooled to −75° C. n-BuLi (2.5 M, 16 mL) was slowly and dropwise added to the reaction solution under nitrogen atmosphere, and the reaction solution was allowed to react at −75° C. for 1 h. Then, a solution of int_130-2 (1.3 g, 3.2 mmol) in THF (25 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (200 mL) was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (1.15 g, yield: 69.6%).

MS (ESI): 518 [M+H]⁺.

### Step 4: Synthesis of Compound Int_130-4

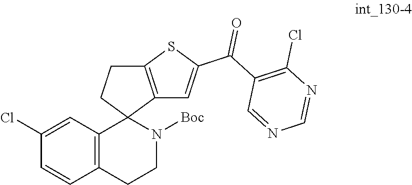 [SMILES: CC(C)(C)OC(=O)N1CCc2ccc(Cl)cc2C12CCc1sc(C(=O)c3cncnc3Cl)cc12]

Int_130-3 (0.200 g, 386 μmol) was dissolved in DCM (10 mL), and a Dess-Martin oxidant (327 mg, 772 μmol, 239 μL) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (170 mg, yield: 85.3%).

¹H NMR (400 MHz, CHLOROFORM-d) δ=9.10 (s, 1H), 8.71 (s, 1H), 7.12 (q, J=8.0 Hz, 2H), 6.86 (s, 1H), 6.58-6.50 (m, 1H), 3.94-3.84 (m, 1H), 3.75-3.63 (m, 1H), 3.34-3.11 (m, 3H), 2.92-2.84 (m, 2H), 2.77-2.68 (m, 1H), 1.24-1.11 (m, 3H), 0.96-0.81 (m, 2H).

MS (ESI): 516 [M+H]⁺.

### Step 5: Synthesis of Compound Int_130-5

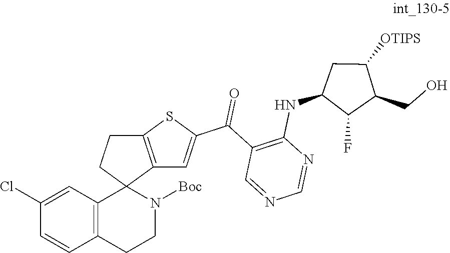

Int_130-4 (403 mg, 782 μmol) and int_88-18 (357 mg, 1.17 mmol) were dissolved in DMF (10 mL), and K₂CO₃ (540 mg, 3.91 mmol) was added to the reaction solution at room temperature. The reaction solution was allowed to react at room temperature for 16 h. LC-MS monitoring showed the reaction was completed. Ice water (50 mL) was added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-20% Ethyl acetate/Petroleum ether gradient) to give a product (480 mg, yield: 78.1%).

MS (ESI): 785 [M+H]⁺.

### Step 6: Synthesis of Compound Int_130-6

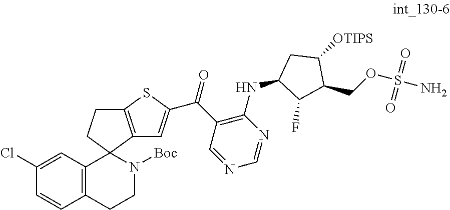

Int_130-5 (0.510 g, 650 μmol) was dissolved in DMF (4 mL), and int_1-17 (150 mg, 1.30 mmol) was added to the reaction solution at room temperature. The reaction solution was allowed to react at room temperature for 1 h. LC-MS monitoring showed the reaction was completed. Ice water (50 mL) was added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (430 mg, yield: 76.6%).

MS (ESI): 864 [M+H]⁺.

### Step 7: Synthesis of Compound Int_130-7

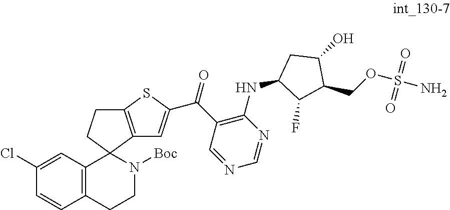 [SMILES: CC(C)(C)OC(=O)N1CCc2ccc(Cl)cc2C12CCc1sc(C(=O)c3cncnc3N[C@H]3C[C@H](O)[C@@H](COS(N)(=O)=O)[C@@H]3F)cc12]

Int_130-6 (432 mg, 500 μmol) was dissolved in THF (4 mL), and TBAF (1 M, 500 μL) was added to the reaction solution at room temperature. The reaction solution was allowed to react at room temperature for 16 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-100% Ethyl acetate/Petroleum ether gradient) to give a solid (238 mg, yield: 67.2%).

MS (ESI): 708 [M+H]⁺.

### Step 8: Synthesis of compound 130

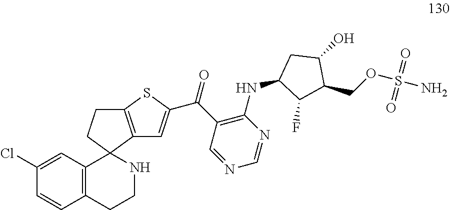 [SMILES: NS(=O)(=O)OC[C@H]1[C@H](F)[C@@H](Nc2ncncc2C(=O)c2cc3c(s2)CCC32NCCc3ccc(Cl)cc32)C[C@@H]1O]

Int_130-7 (177 mg, 250 μmol) was added to TFA (0.500 mL) at room temperature, and the reaction solution was allowed to react at room temperature for 10 min. LC-MS monitoring showed the reaction was completed. Ammonia water (1 mL) and water (10 mL) were added to the reaction solution. The aqueous phase was extracted with dichloromethane (10 mL×3), and the organic phase was dried over anhydrous sodium sulfate. The organic phase was concentrated under reduced pressure to give a crude product. The crude product was purified by preparative HPLC to give a solid (55 mg, yield: 36.1%).

MS (ESI): 608 [M+H]⁺.

### Example 12. Synthesis of Compound 131 and Compound 132

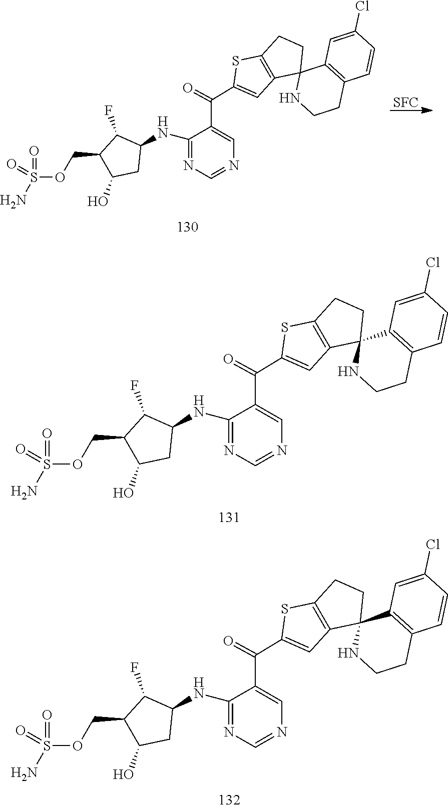

Compound 130 (50 mg, 82.2 μmol) was subjected to SFC chiral resolution (column: DAICEL CHIRALPAK AD (250 mm×30 mm, 10 μm); mobile phase: [CO₂-EtOH (0.1% NH₃H₂O)]; B %: 60%, isocratic elution mode) to give compound 131 (21 mg) and compound 132 (18 mg).

Compound 131: ¹H NMR (400 MHz, DMSO-d6) δ 8.60 (s, 1H) 8.56 (s, 1H) 8.35 (d, J=8.19 Hz, 1H) 7.53 (s, 2H) 7.26 (s, 1H) 7.10-7.21 (m, 2H) 6.79 (d, J=1.71 Hz, 1H) 5.19 (d, J=4.65 Hz, 1H) 5.00-5.13 (m, 1H) 4.81-4.99 (m, 1H) 4.14 (qd, J=9.80, 6.05 Hz, 2H) 3.94 (br s, 1H) 3.15 (dt, J=7.52, 3.58 Hz, 2H) 3.04-3.10 (m, 1H) 2.93-3.01 (m, 1H) 2.59-2.87 (m, 5H) 2.19-2.31 (m, 1H) 1.94-2.05 (m, 1H) 1.80-1.94 (m, 1H); MS (ESI): 608 [M+H]⁺.

Analytical SFC retention time: 1.298 min (Instrument: Waters UPCC with PDA Detector; Column: Chiralpak AD-3 50×4.6 mm I.D., 3 μm; Mobile phase: A: CO₂, B: Ethanol (0.05% DEA); Isocratic: 40% B; Flow rate: 4 mL/min; Column temp: 35° C.; ABPR: 1500 psi).

Compound 132: ¹H NMR (400 MHz, DMSO-d6) δ=8.59 (s, 1H) 8.54 (s, 1H) 8.29 (d, J=8.31 Hz, 1H) 7.54 (s, 2H) 7.24 (s, 1H) 7.11-7.21 (m, 2H) 6.78 (d, J=1.47 Hz, 1H) 5.18 (d, J=4.65 Hz, 1H) 5.00-5.11 (m, 1H) 4.80-4.99 (m, 1H) 4.07-4.20 (m, 2H) 3.92 (br s, 1H) 3.12-3.18 (m, 2H) 3.04-3.11 (m, 1H) 2.93-3.02 (m, 1H) 2.59-2.87 (m, 5H) 2.20-2.31 (m, 1H) 1.89-2.00 (m, 1H) 1.76-1.89 (m, 1H); MS (ESI): 608 [M+H]⁺.

Analytical SFC retention time: 0.461 min (Instrument: Waters UPCC with PDA Detector; Column: Chiralpak AD-3 50x4.6 mm I.D., 3 um; Mobile phase: A: CO₂, B: Ethanol (0.05% DEA); Isocratic: 40% B; Flow rate: 4 mL/min; Column temp: 35° C.; ABPR: 1500 psi).

### Example 13. Synthesis of Compound 162

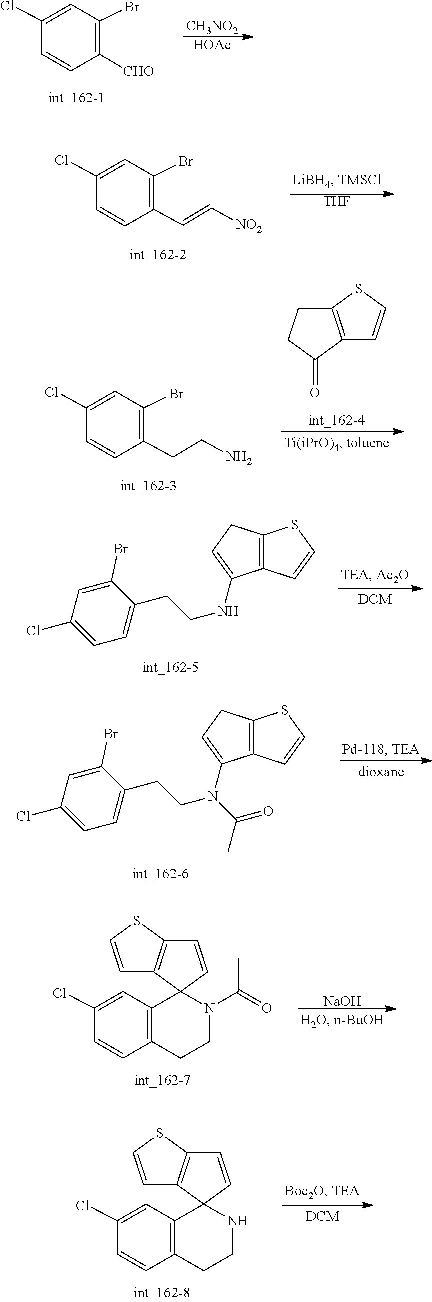

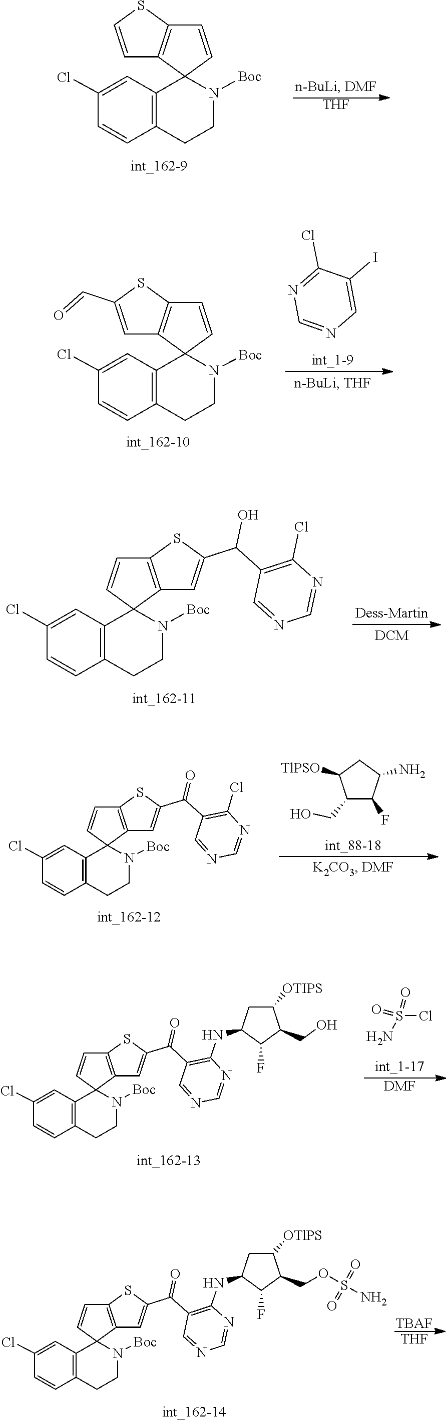

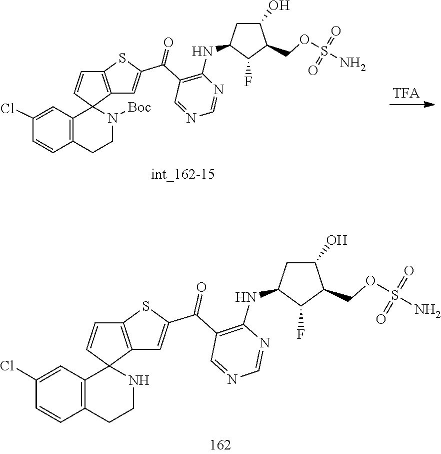 [SMILES: CC(C)(C)OC(=O)N1CCc2ccc(Cl)cc2C12C=Cc1sc(C(=O)c3cncnc3N[C@H]3C[C@H](O)[C@@H](COS(N)(=O)=O)[C@@H]3F)cc12 ; NS(=O)(=O)OC[C@H]1[C@H](F)[C@@H](Nc2ncncc2C(=O)c2cc3c(s2)C=CC32NCCc3ccc(Cl)cc32)C[C@@H]1O]

### Step 1: Synthesis of Compound Int_162-2

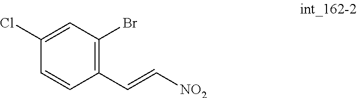 [SMILES: O=[N+]([O-])/C=C/c1ccc(Cl)cc1Br]

Int_162-1 (50.0 g, 228 mmol) and NH₄OAc (40.4 g, 524 mmol) were dissolved in acetic acid (500 mL), and the mixed solution was purged with nitrogen three times. CH₃NO₂ (36.2 g, 593 mmol, 32.0 mL) was added to the reaction solution under nitrogen atmosphere, and the reaction solution was heated to 100° C. and stirred for 16 h. LC-MS monitoring showed the reaction was completed. The reaction solution was cooled to room temperature and then poured slowly into 1000 mL of ice water. The aqueous phase was extracted with ethyl acetate (800 mL×3), and the organic phase was dried over anhydrous sodium sulfate. The organic phase was concentrated under reduced pressure to give a crude product (50 g, yield: 83.6%), which was directly used in the next step.

¹H NMR (400 MHz, CHLOROFORM-d) δ=8.35 (d, J=13.6 Hz, 1H), 7.73 (d, J=2.0 Hz, 1H), 7.58-7.51 (m, 2H), 7.40 (dd, J=2.0, 8.4 Hz, 1H).

### Step 2: Synthesis of Compound Int_162-3

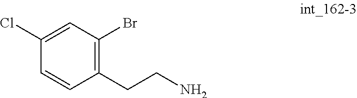 [SMILES: NCCc1ccc(Cl)cc1Br]

LiBH₄ (20.8 g, 952 mmol) was dissolved in tetrahydrofuran (300 mL), and TMSCl (207 g, 1.90 mol, 242 mL) was added to the reaction solution at 0° C. under nitrogen atmosphere. The reaction solution was stirred at 0° C. for 30 min, and a solution of int_162-2 (25.0 g, 95.2 mmol) in tetrahydrofuran (250 mL) was slowly and dropwise added to the reaction solution within 30 min. The reaction solution was warmed to 70° C. and allowed to react for another 2 h. LC-MS monitoring showed the reaction was completed. The reaction solution was cooled to 0° C., and 200 mL of methanol was slowly added to quench the reaction. The organic phase was concentrated under reduced pressure to give a crude product, and the crude product was adjusted to pH>7 with ammonia water. The aqueous phase was extracted with ethyl acetate (500 mL×3), and the organic phase was dried over anhydrous sodium sulfate. The organic phase was concentrated under reduced pressure to give a crude product (20 g), which was directly used in the next step.

ESI-MS m/z: 234 [M+H]⁺.

### Step 3: Synthesis of Compound Int_162-5

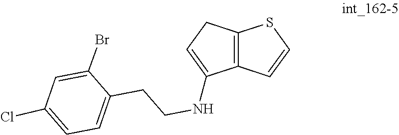 [SMILES: Clc1ccc(CCNC2=CCc3sccc32)c(Br)c1]

Int_162-3 (20.0 g, 85.3 mmol), int_162-4 (9.07 g, 65.6 mmol), and Ti(i-PrO)₄ (37.3 g, 131 mmol) were dissolved in methylbenzene (200 mL). The mixed solution was purged with nitrogen three times, heated to 90° C., and stirred for 2 h. LC-MS monitoring showed the reaction was completed. The reaction solution was cooled to room temperature and concentrated under reduced pressure to give a crude product (23 g), which was directly used in the next step.

ESI-MS m/z: 354 [M+H]⁺.

### Step 4: Synthesis of Compound Int_162-6

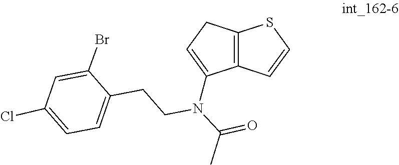 [SMILES: CC(=O)N(CCc1ccc(Cl)cc1Br)C1=CCc2sccc21]

Int_162-5 (23.0 g, 64.9 mmol), TEA (65.6 g, 648 mmol, 90.3 mL), and Ac2O (33.1 g, 324 mmol, 30.5 mL) were dissolved in dichloromethane (400 mL), and the reaction solution was allowed to react at 20° C. for 2 h under nitrogen atmosphere. LC-MS monitoring showed the reaction was completed. The reaction solution was cooled to room temperature. The organic phase was concentrated under reduced pressure to give a crude product, and the crude product was adjusted to pH>7 with a saturated aqueous sodium bicarbonate solution. The aqueous phase was extracted with dichloromethane (500 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (20 g, yield: 77.7%).

¹H NMR (400 MHz, DMSO-d6) δ=1.09-1.17 (m, 1H), 1.88 (br d, J=7.00 Hz, 3H), 2.82-2.96 (m, 2H), 3.41-3.48 (m, 2H), 3.80 (br t, J=7.13 Hz, 2H), 3.99 (q, J=7.13 Hz, 1H), 7.18-7.38 (m, 2H), 7.43-7.54 (m, 1H), 7.63 (d, J=2.13 Hz, 1H).

### Step 5: Synthesis of Compound Int_162-7

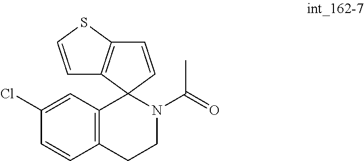 [SMILES: CC(=O)N1CCc2ccc(Cl)cc2C12C=Cc1sccc12]

Int_162-6 (20.0 g, 50.4 mmol), DIPEA (13.0 g, 101 mmol, 17.6 mL), and ditert-butyl(cyclopentyl)phosphane;dichloropalladium;iron (3.29 g, 5.04 mmol) were dissolved in 1,4-dioxane (300 mL). The reaction solution was purged with nitrogen three times, warmed to 110° C., and allowed to react for 7 h. LC-MS monitoring showed the reaction was completed. The reaction solution was cooled to room temperature and then poured slowly into 300 mL of ice water. The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (9.4 g, yield: 59%).

¹H NMR (400 MHz, DMSO-d6) δ=1.69-1.96 (m, 3H), 2.91-3.01 (m, 2H), 3.35 (s, 11H), 3.73-3.86 (m, 1H), 3.89-3.99 (m, 1H), 6.40-6.47 (m, 1H), 6.49-6.60 (m, 1H), 6.77-6.85 (m, 1H), 6.87-6.94 (m, 1 H), 7.15-7.21 (m, 1H), 7.23-7.31 (m, 1H), 7.39-7.49 (m, 1H).

ESI-MS m/z: 316 [M+H]⁺.

### Step 6: Synthesis of Compound Int_162-8

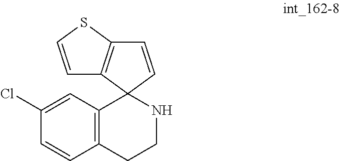 [SMILES: Clc1ccc2c(c1)C1(C=Cc3sccc31)NCC2]

Int_162-7 (9.40 g, 29.8 mmol) was dissolved in a mixed solvent of n-butanol (15 mL) and water (5 mL), and sodium hydroxide (23.8 g, 595 mmol) was added to the reaction solution. The reaction solution was warmed to 100° C. and allowed to react for 16 h. Subsequently, the reaction solution was warmed to room temperature and allowed to react for 1 h. LC-MS monitoring showed the reaction was completed. Water (200 mL) was added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography (SiO₂, PE/EtOAc=1/4) to give a solid (8 g, yield: 98.2%).

¹H NMR (500 MHz, DMSO-d6) δ=1.99 (s, 1H), 2.94-3.00 (m, 1H), 3.08-3.17 (m, 1H), 3.19-3.27 (m, 1H), 3.36-3.43 (m, 1H), 6.44 (d, J=2.14 Hz, 1H), 6.64 (dd, J=5.57, 1.45 Hz, 1H), 6.80 (d, J=5.49 Hz, 1H), 6.94 (d, J=4.88 Hz, 1H), 7.12-7.20 (m, 2H), 7.38 (dd, J=4.81, 1.45 Hz, 1H).

ESI-MS m/z: 274 [M+H]⁺.

### Step 7: Synthesis of Compound Int_162-9

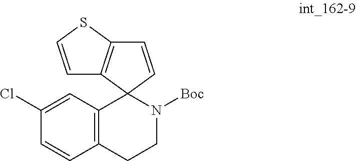 [SMILES: CC(C)(C)OC(=O)N1CCc2ccc(Cl)cc2C12C=Cc1sccc12]

Int_162-8 (8.00 g, 29.2 mmol) and (Boc)₂O (9.57 g, 43.8 mmol, 10.1 mL) were dissolved in dichloromethane (90 mL), and TEA (8.87 g, 87.7 mmol, 12.2 mL) was added to the reaction solution at room temperature. The reaction solution was allowed to react at room temperature for 16 h under nitrogen atmosphere. LC-MS monitoring showed the reaction was completed. The reaction solution was poured slowly into 100 mL of ice water. The aqueous phase was extracted with dichloromethane (100 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (8.9 g, yield: 81.5%).

¹H NMR (400 MHz, DMSO-d6) δ=1.13 (s, 9H), 2.88-3.06 (m, 2H), 3.72-3.91 (m, 2H), 6.47-6.63 (m, 2H), 6.79 (d, J=5.50 Hz, 1H), 6.85-6.95 (m, 1H), 7.16-7.29 (m, 2H), 7.41-7.49 (m, 1H).

### Step 8: Synthesis of Compound Int_162-10

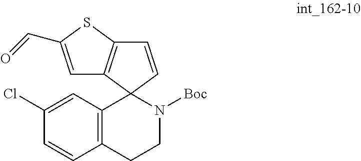 [SMILES: CC(C)(C)OC(=O)N1CCc2ccc(Cl)cc2C12C=Cc1sc(C=O)cc12]

Int_162-9 (0.30 g, 798 μmol) was dissolved in THF (10 mL), and the mixture was cooled to −60° C. n-BuLi (2.50 M, 479 μL) was slowly and dropwise added to the reaction solution under nitrogen atmosphere, and the reaction solution was allowed to react at −60° C. for 1 h. Then, DMF (175 mg, 2.39 mmol, 184 μL) was dropwise added to the reaction solution at −60° C., and after the dropwise addition was completed, the reaction solution was allowed to react at −60° C. for 3 h. LC-MS monitoring showed the reaction was completed. A saturated ammonium chloride solution (100 mL) was slowly added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (140 mg, yield: 43.4%).

¹H NMR (400 MHz, DMSO-d6) δ=1.12 (s, 9H), 2.88-3.08 (m, 2H), 3.66-3.93 (m, 2H), 6.53-6.59 (m, 1H), 6.90 (d, J=5.62 Hz, 1H), 6.94-7.00 (m, 1H), 7.19-7.26 (m, 1H), 7.27-7.33 (m, 1H), 7.74-7.85 (m, 1H), 9.75 (s, 1H).

MS (ESI): 402 [M+H]⁺.

### Step 9: Synthesis of Compound Int_162-11

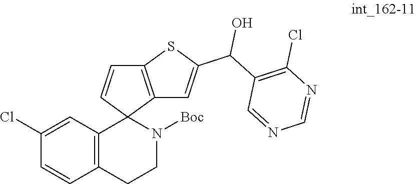 [SMILES: CC(C)(C)OC(=O)N1CCc2ccc(Cl)cc2C12C=Cc1sc(C(O)c3cncnc3Cl)cc12]

Int_1-9 (4.79 g, 19.91 mmol) was dissolved in THF (50 mL), and the mixture was cooled to −75° C. n-BuLi (2.5 M, 16 mL) was slowly and dropwise added to the reaction solution under nitrogen atmosphere, and the reaction solution was allowed to react at −75° C. for 1 h. Then, a solution of int_162-10 (1.33 g, 3.31 mmol) in THF (25 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (200 mL) was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (1.21 g, yield: 71.1%).

¹H NMR (400 MHz, DMSO-d6) δ=1.14 (s, 9H), 2.82-3.02 (m, 3H), 3.67-3.82 (m, 2H), 5.74-5.78 (m, 1H), 6.53-6.59 (m, 1H), 6.90-6.94 (m, 1H), 6.98-7.03 (m, 1H), 7.19-7.29 (m, 2H), 7.57-7.62 (m, 1 H), 8.97-9.01 (m, 1H), 9.19 (s, 1H).

MS (ESI): 516 [M+H]⁺.

### Step 10: Synthesis of Compound Int_162-12

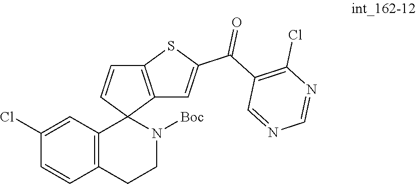 [SMILES: CC(C)(C)OC(=O)N1CCc2ccc(Cl)cc2C12C=Cc1sc(C(=O)c3cncnc3Cl)cc12]

Int_162-11 (1.60 g, 3.10 mmol) was dissolved in DCM (100 mL), and a Dess-Martin oxidant (2.63 g, 6.20 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (SiO₂, PE/EtOAc=3/1) to give a solid (0.9 g, yield: 56.5%).

MS (ESI): 514 [M+H]⁺.

### Step 11: Synthesis of Compound Int_162-13

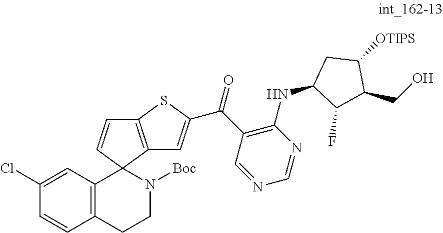

Int_162-12 (0.90 g, 1.75 mmol) and int_88-18 (1.59 g, 5.22 mmol) were dissolved in DMF (20 mL), and K₂CO₃ (1.21 g, 8.75 mmol) was added to the reaction solution at room temperature. The reaction solution was warmed to 50° C. and allowed to react for 1 h. LC-MS monitoring showed the reaction was completed. Ice water (100 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-30% THF/Petroleum ether gradient) to give a product (0.7 g, yield: 51%).

MS (ESI): 783 [M+H]⁺.

### Step 12: Synthesis of Compound Int 162-14

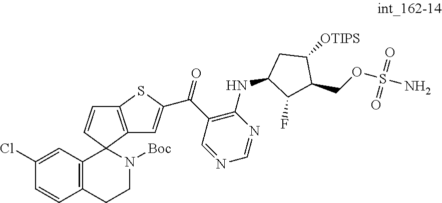

Int_162-13 (0.716 g, 914 μmol) was dissolved in DMF (20 mL), and int 1-17 (423 mg, 3.66 mmol) was added to the reaction solution at room temperature. The reaction solution was allowed to react at room temperature for 1 h. LC-MS monitoring showed the reaction was completed. Ice water (20 mL) was added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography to give a solid (770 mg, yield: 97.7%).

MS (ESI): 862 [M+H]⁺.

### Step 13: Synthesis of Compound Int_162-15

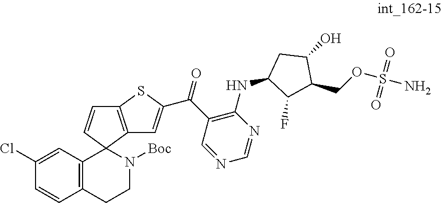 [SMILES: CC(C)(C)OC(=O)N1CCc2ccc(Cl)cc2C12C=Cc1sc(C(=O)c3cncnc3N[C@H]3C[C@H](O)[C@@H](COS(N)(=O)=O)[C@@H]3F)cc12]

Int_162-14 (786 mg, 912 μmol) was dissolved in THF (20 mL), and TBAF (1 M, 912 μL) was added to the reaction solution at room temperature. The reaction solution was allowed to react at room temperature for 16 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-30% Ethyl acetate/Petroleum ether gradient) to give a solid (400 mg, yield: 62.1%).

MS (ESI): 706 [M+H]⁺.

### Step 14: Synthesis of Compound 162

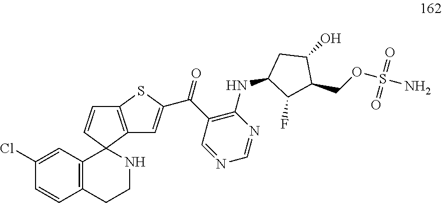 [SMILES: NS(=O)(=O)OC[C@H]1[C@H](F)[C@@H](Nc2ncncc2C(=O)c2cc3c(s2)C=CC32NCCc3ccc(Cl)cc32)C[C@@H]1O]

Int_162-15 (205 mg, 291 μmol) was added to TFA (0.5 mL) at room temperature, and the reaction solution was allowed to react at room temperature for 10 min. LC-MS monitoring showed the reaction was completed. A saturated aqueous sodium bicarbonate solution was added to the reaction solution to adjust the pH to 8, and then the reaction solution was purified by preparative HPLC to give a solid (90 mg, yield: 51.1%).

MS (ESI): 606 [M+H]⁺.

### Example 14. Synthesis of Compound 343 and Compound 344

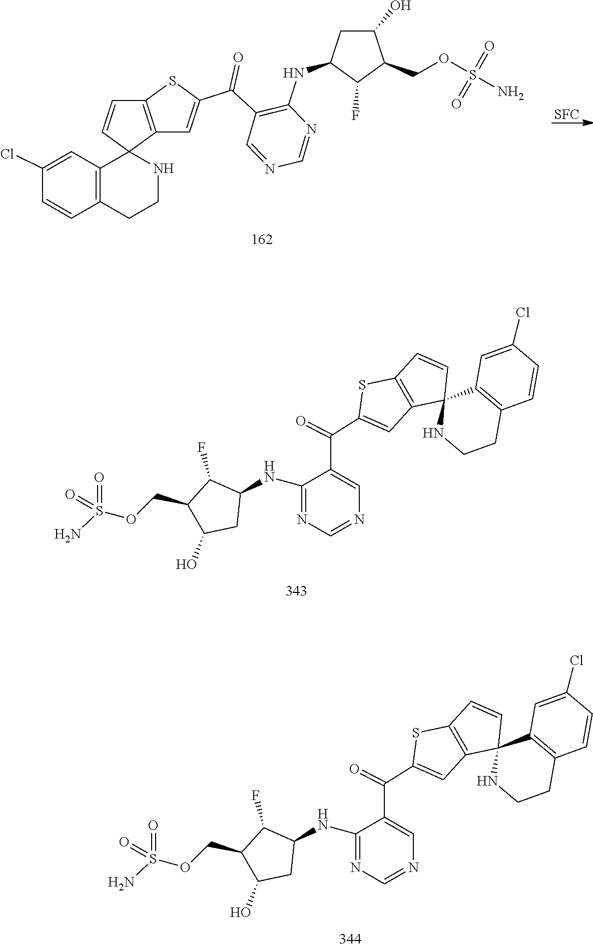

Compound 162 (100 mg, 164 μmol) was subjected to SFC chiral resolution (column: DAICEL CHIRALPAK IG (250 mm×30 mm, 10 μm); mobile phase: [CO₂-EtOH (0.1% NH₃H₂O)]; B %: 50%, isocratic elution mode) to give compound 343 (25 mg) and compound 344 (20 mg).

Compound 343: ¹H NMR (400 MHz, DMSO-d6) a=8.59 (s, 1H), 8.50 (s, 1H), 8.12-8.19 (m, 1H), 7.57 (s, 1H), 7.53 (s, 2H), 7.14-7.22 (m, 2H), 6.97 (s, 2H), 6.46 (s, 1H), 5.17 (br s, 1H), 4.98-5.10 (m, 1H), 4.78-4.97 (m, 1H), 4.08-4.17 (m, 2H), 3.90 (br s, 1H), 3.22 (br d, J=6.97 Hz, 2H), 3.09-3.14 (m, 1H), 2.77-2.90 (m, 2H), 2.20-2.29 (m, 1H), 1.91 (br d, J=9.78 Hz, 1H), 1.74-1.83 (m, 1H); MS (ESI): 606 [M+H]⁺.

Analytical SFC retention time: 2.439 min (Instrument: Waters UPCC with PDA Detector; Column: Chiralpak IG-3 100x4.6 mm I.D., 3 um; Mobile phase: A: CO₂, B: Ethanol (0.05% DEA); Isocratic: 40% B; Flow rate: 2.8 mL/min; Column temp: 35° C.; ABPR: 1500 psi).

Compound 344: ¹H NMR (400 MHz, DMSO-d6) a=8.59 (s, 1H), 8.50 (s, 1H), 8.12-8.19 (m, 1H), 7.57 (s, 1H), 7.53 (s, 2H), 7.14-7.22 (m, 2H), 6.97 (s, 2H), 6.46 (s, 1H), 5.17 (br s, 1H), 4.98-5.10 (m, 1H), 4.78-4.97 (m, 1H), 4.08-4.17 (m, 2H), 3.90 (br s, 1H), 3.22 (br d, J=6.97 Hz, 2H), 3.09-3.14 (m, 1H), 2.77-2.90 (m, 2H), 2.20-2.29 (m, 1H), 1.91 (br d, J=9.78 Hz, 1H), 1.74-1.83 (m, 1H); MS (ESI): 606 [M+H]⁺.

Analytical SFC retention time: 1.920 min (Instrument: Waters UPCC with PDA Detector; Column: Chiralpak IG-3 100×4.6 mm I.D., 3 μm; Mobile phase: A: CO₂, B: Ethanol (0.05% DEA); Isocratic: 40% B; Flow rate: 2.8 mL/min; Column temp: 35° C.; ABPR: 1500 psi).

### Example 15. Synthesis of Compound 190

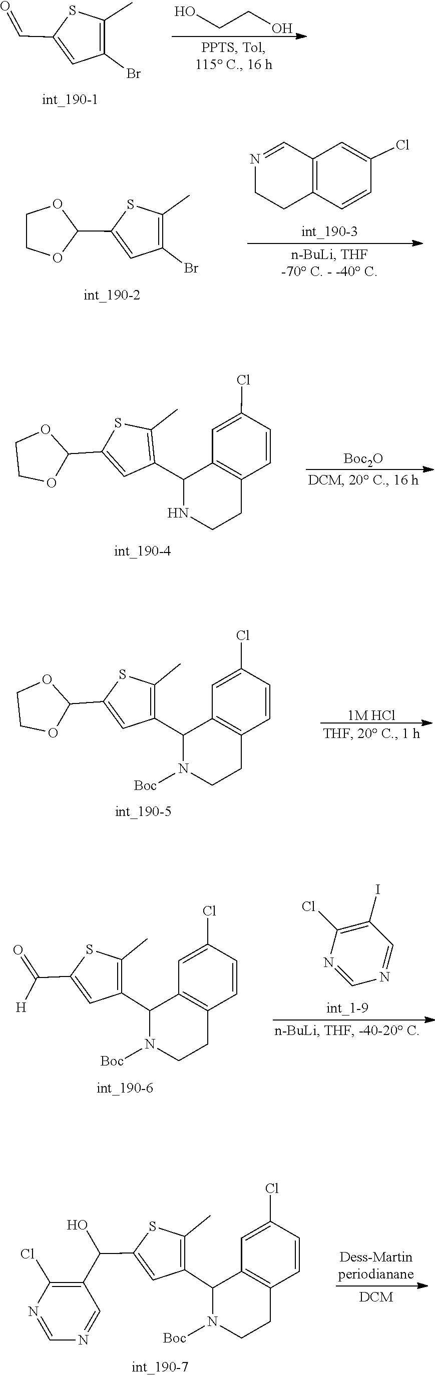

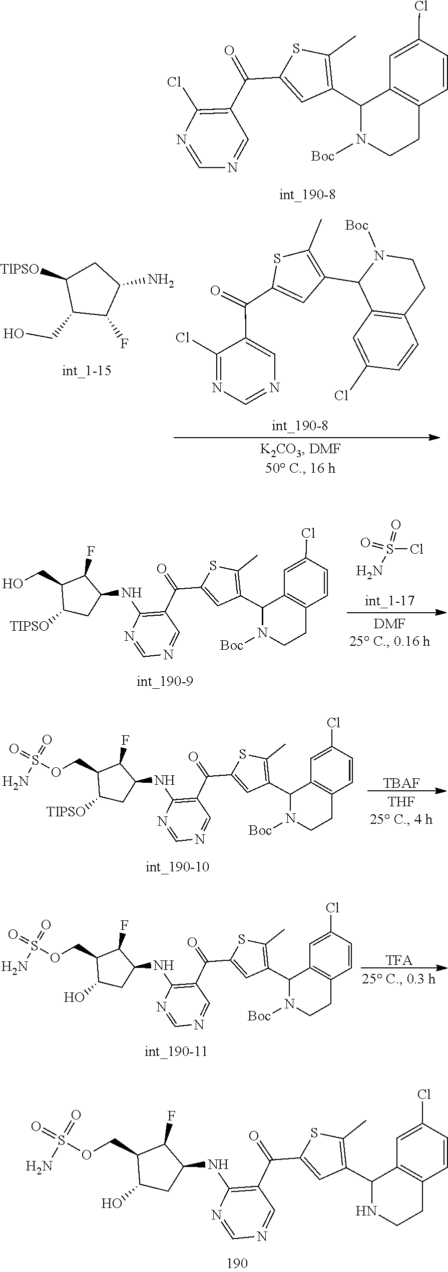

### Step 1: Synthesis of Compound Int_190-2

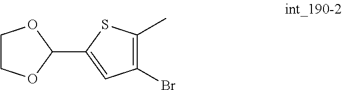

Int_190-1 (20.0 g, 97.5 mmol), PPTS (245 mg, 975 μmol), and ethylene glycol (18.2 g, 293 mmol, 16.4 mL) were dissolved in methylbenzene (300 mL). The mixed solution was purged with nitrogen three times, heated to 115° C., and allowed to react for 16 h. LC-MS monitoring showed the reaction was completed. The reaction solution was cooled to room temperature, and concentrated under reduced pressure to give a crude product. The crude product was purified by preparative column chromatography (ISCO®; 220 g SepaFlash® Silica Flash Column, Eluent of 0-10% Ethyl acetate/Petroleum ether gradient) to give a solid (23 g, yield: 94.7%).

¹H NMR (400 MHz, CHLOROFORM-d) δ=6.93-6.83 (m, 1H), 5.93 (s, 1H), 4.06-3.99 (m, 2H), 3.96-3.88 (m, 2H), 2.35-2.27 (m, 3H).

### Step 2: Synthesis of Compound Int_190-4

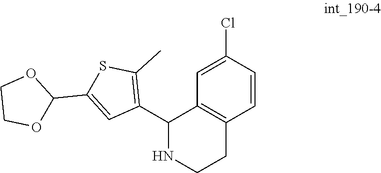 [SMILES: Cc1sc(C2OCCO2)cc1C1NCCc2ccc(Cl)cc21]

Int_190-2 (20.5 g, 82.1 mmol) was dissolved in THF (100 mL), and the mixture was cooled to −70° C. n-BuLi (2.5 M, 38.6 mL) was slowly and dropwise added to the reaction solution under nitrogen atmosphere, and the reaction solution was allowed to react at −70° C. for 0.5 h. At −70° C., int_190-3 (8.00 g, 48.3 mmol) was dissolved in THF (50 mL), and the mixture was cooled to −40° C. BF₃·Et₂O (7.54 g, 53.1 mmol, 6.56 mL) was slowly added dropwise to the reaction solution under nitrogen atmosphere. The reaction solution was allowed to react at −40° C. for 10 min, and the previously prepared lithium reagent was slowly added dropwise to the reaction solution with the temperature maintained at −40° C. After the dropwise addition was completed, the reaction solution was slowly warmed to room temperature and allowed to react for 1 h. LC-MS monitoring showed the reaction was completed. A saturated ammonium chloride solution (300 mL) was slowly added to the reaction solution. The aqueous phase was extracted with ethyl acetate (450 mL×3), and the organic phase was dried over anhydrous sodium sulfate. The organic phase was concentrated under reduced pressure to give a crude product. The crude product was purified by preparative column chromatography (ISCO®; 120 g SepaFlash® Silica Flash Column, Eluent of 0-100% Ethyl acetate/Petroleum ether gradient) to give a solid (8 g, yield: 49.3%).

MS (ESI): 336 [M+H]⁺.

### Step 3: Synthesis of Compound Int_190-5

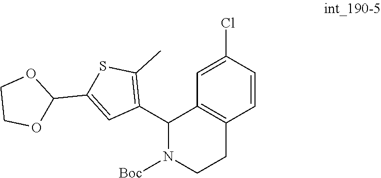 [SMILES: Cc1sc(C2OCCO2)cc1C1c2cc(Cl)ccc2CCN1C(=O)OC(C)(C)C]

Int_190-4 (8.00 g, 23.8 mmol) and (Boc)₂O (6.24 g, 28.6 mmol, 6.57 mL) were dissolved in DCM (60 mL), and TEA (2.89 g, 28.6 mmol) was added to the reaction solution at room temperature. The reaction solution was allowed to react at room temperature for 16 h. LC-MS monitoring showed the reaction was completed. The reaction solution was cooled to room temperature, and concentrated under reduced pressure to give a crude product. The crude product was purified by preparative column chromatography (ISCO®; 80 g SepaFlash® Silica Flash Column, Eluent of 0-20% Ethyl acetate/Petroleum ether gradient) to give a solid (5 g, yield: 48.1%).

¹H NMR (400 MHz, CHLOROFORM-d) δ=7.21-7.08 (m, 2H), 6.96 (s, 1H), 6.54 (s, 1H), 6.26 (br s, 1H), 5.88 (s, 1H), 5.32 (s, 1H), 4.29-4.05 (m, 3H), 4.01-3.93 (m, 2H), 3.17 (dt, J=3.9, 12.9 Hz, 1H), 3.07-2.91 (m, 1H), 2.80-2.67 (m, 1H), 2.56 (s, 3H), 1.48 (s, 8H).

### Step 4: Synthesis of Compound Int_190-6

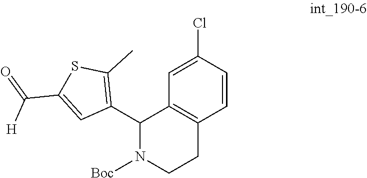 [SMILES: Cc1sc(C=O)cc1C1c2cc(Cl)ccc2CCN1C(=O)OC(C)(C)C]

Int_190-5 (5.00 g, 11.5 mmol) was dissolved in THF (60 mL), and an aqueous hydrochloric acid solution (1 M, 30.00 mL) was added dropwise to the reaction solution. The reaction solution was allowed to react at room temperature for 1 h. LC-MS monitoring showed the reaction was completed. Water (200 mL) was slowly added to the reaction solution. The aqueous phase was extracted with dichloromethane (20 mL×3), and the organic phase was dried over anhydrous sodium sulfate. The organic phase was concentrated under reduced pressure to give a crude product. The crude product was purified by preparative column chromatography (ISCO®; 80 g SepaFlash® Silica Flash Column, Eluent of 0-20% Ethyl acetate/Petroleum ether gradient) to give an oil (4.4 g, yield: 97.9%).

¹H NMR (400 MHz, CHLOROFORM-d) δ=9.67 (s, 1H), 7.25-7.19 (m, 1H), 7.18-7.15 (m, 1H), 7.12 (s, 1H), 6.96 (d, J=1.8 Hz, 1H), 6.34 (br s, 1H), 4.15 (q, J=7.1 Hz, 1H), 3.12 (dt, J=3.8, 12.8 Hz, 1H), 3.04-2.93 (m, 1H), 2.77 (br d, J=16.3 Hz, 1H), 1.53-1.47 (m, 9H).

### Step 5: Synthesis of Compound Int_190-7

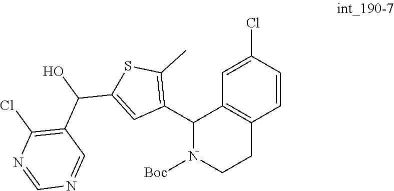 [SMILES: Cc1sc(C(O)c2cncnc2Cl)cc1C1c2cc(Cl)ccc2CCN1C(=O)OC(C)(C)C]

Int_1-9 (5.40 g, 22.5 mmol) was dissolved in THF (50 mL), and the mixture was cooled to −40° C. n-BuLi (2.5 M, 18.0 mL) was slowly and dropwise added to the reaction solution under nitrogen atmosphere, and the reaction solution was allowed to react at −40° C. for 0.5 h. Then, a solution of int_190-6 (4.40 g, 11.2 mmol) in THE (20 mL) was dropwise added to the reaction solution at −40° C., and after the dropwise addition was completed, the reaction solution was slowly warmed to room temperature and allowed to react for 1.5 h. LC-MS monitoring showed the reaction was completed. A saturated ammonium chloride solution (100 mL) was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography (ISCO®; 80 g SepaFlash® Silica Flash Column, Eluent of 0-30% Ethyl acetate/Petroleum ether gradient) to give an oil (4 g, yield: 70.4%).

¹H NMR (400 MHz, CHLOROFORM-d) δ=8.93 (s, 1H), 8.84 (s, 1H), 7.12-7.03 (m, 2H), 6.90-6.83 (m, 1H), 6.40 (d, J=5.5 Hz, 1H), 6.15 (br s, 1H), 6.06-5.99 (m, 1H), 3.08-2.98 (m, 1H), 2.94-2.83 (m, 1H), 2.69-2.59 (m, 2H), 2.42 (s, 3H), 1.37 (s, 9H).

### Step 6: Synthesis of Compound Int_190-8

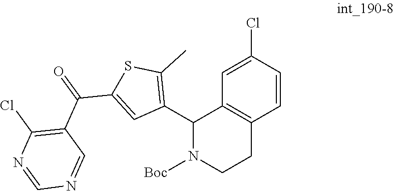 [SMILES: Cc1sc(C(=O)c2cncnc2Cl)cc1C1c2cc(Cl)ccc2CCN1C(=O)OC(C)(C)C]

Int_190-7 (600 mg, 1.18 mmol) was dissolved in DCM (6 mL), and a Dess-Martin oxidant (766.46 mg, 1.81 mmol) was added to the reaction solution at room temperature. The reaction solution was allowed to react at room temperature for 1 h. LC-MS monitoring showed the reaction was completed. A saturated aqueous sodium bicarbonate solution was slowly added to the reaction solution to adjust the pH to about 8. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (530 mg, yield: 89%).

MS (ESI): 504 [M+H]⁺.

### Step 7: Synthesis of Compound Int_190-9

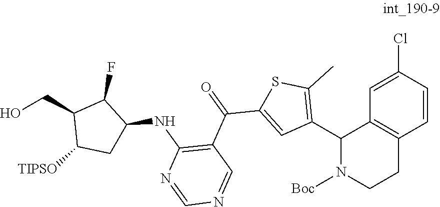

Int_1-15 (0.500 g, 1.64 mmol) and int_190-8 (826 mg, 1.64 mmol) were dissolved in DMF (10 mL), and K₂CO₃ (679 mg, 4.91 mmol) was added to the reaction solution at room temperature. The reaction solution was warmed to 50° C. and allowed to react for 1 h. LC-MS monitoring showed the reaction was completed. Ice water (10 mL) was added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-30% THF/Petroleum ether gradient) to give a product (400 mg, yield: 28.3%).

MS (ESI): 773 [M+H]⁺.

### Step 8: Synthesis of Compound Int_190-10

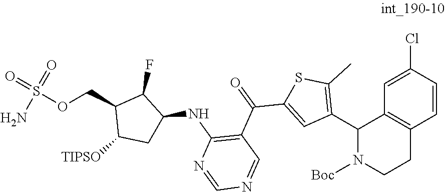

Int_190-9 (0.180 g, 233 μmol) was dissolved in DMF (2 mL), and int_1-17 (269 mg, 2.33 mmol) was added to the reaction solution at room temperature. The reaction solution was allowed to react at room temperature for 1 h. LC-MS monitoring showed the reaction was completed. Ice water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography to give a solid (178 mg, yield: 89.8%).

MS (ESI): 852 [M+H]⁺.

### Step 9: Synthesis of Compound Int_1-11

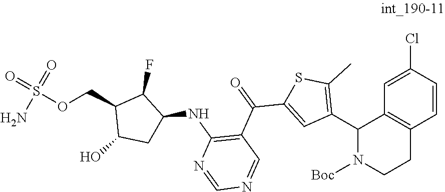 [SMILES: Cc1sc(C(=O)c2cncnc2N[C@H]2C[C@H](O)[C@@H](COS(N)(=O)=O)[C@H]2F)cc1C1c2cc(Cl)ccc2CCN1C(=O)OC(C)(C)C]

Int_190-10 (0.500 g, 586 μmol) was dissolved in THF (5 mL), and TBAF (1 M, 1.17 mL) was added to the reaction solution at room temperature. The reaction solution was allowed to react at room temperature for 1 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-100% Ethyl acetate/Petroleum ether gradient) to give a solid (150 mg, yield: 35.6%).

MS (ESI): 696 [M+H]⁺.

### Step 10: Synthesis of Compound 190

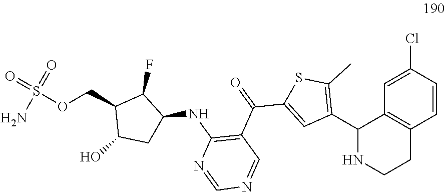 [SMILES: Cc1sc(C(=O)c2cncnc2N[C@H]2C[C@H](O)[C@@H](COS(N)(=O)=O)[C@H]2F)cc1C1NCCc2ccc(Cl)cc21]

Int_190-11 (0.15 g, 209 μmol) was added to TFA (1 mL) at room temperature, and the reaction solution was allowed to react at room temperature for 5 min. LC-MS monitoring showed the reaction was completed. A saturated aqueous sodium bicarbonate solution was added to the reaction solution to adjust the pH to 8, and then the reaction solution was purified by preparative HPLC (column: Boston Prime C18 150×30 mm×5 μm; mobile phase: [water (ammonia hydroxide v/v)-ACN]; gradient: 33%-53% B over 11 min) to give a solid (40 mg, yield: 32.1%).

MS (ESI): 596 [M+H]⁺.

### Example 16. Synthesis of Compound 191 and Compound 192

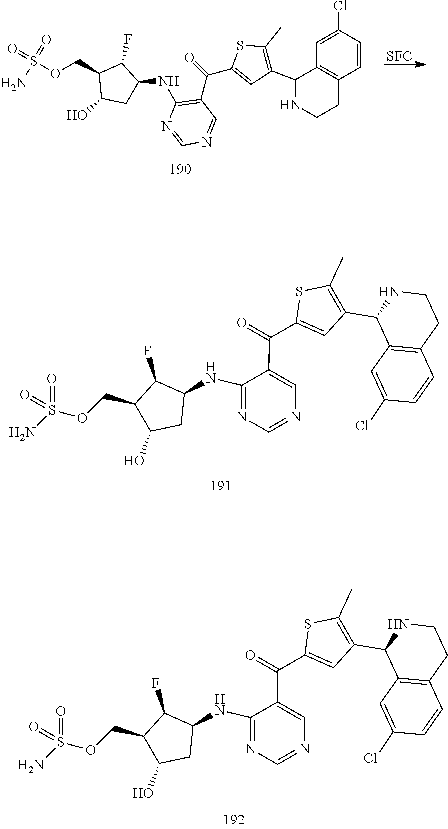

Compound 190 (50 mg, 170 μmol) was subjected to SFC chiral resolution (column: DAICEL CHIRALPAK IG (250 mm×30 mm, 10 μm); mobile phase: [CO₂-MeOH (0.1% NH₃H₂O)]) to give compound 191 (10 mg) and compound 192 (16 mg).

Compound 191: MS (ESI): 596 [M+H]⁺.

Compound 192: MS (ESI): 596 [M+H]⁺.

### Example 17. Synthesis of Compound 232

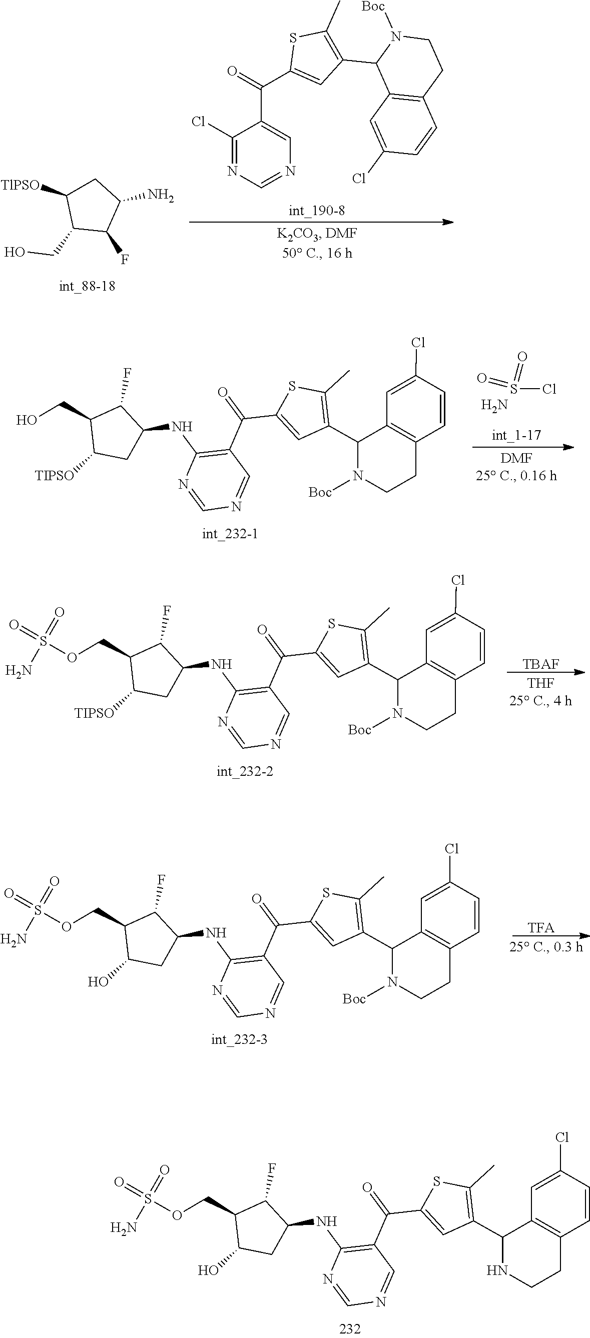

### Step 1: Synthesis of Compound Int_232-1

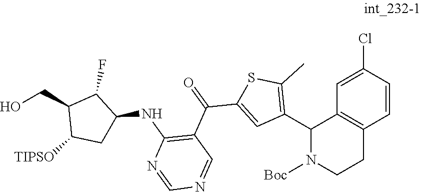

Int_88-18 (0.93 g, 3.04 mmol) and int 190-8 (1.54 g, 3.04 mmol) were dissolved in DMF (10 mL), and K₂CO₃ (1.26 g, 9.13 mmol) was added to the reaction solution at room temperature. The reaction solution was warmed to 50° C. and allowed to react for 5 h. LC-MS monitoring showed the reaction was completed. Ice water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-15% THF/Petroleum ether gradient) to give a product (650 mg, yield: 27.6%).

MS (ESI): 773 [M+H]⁺.

### Step 2: Synthesis of Compound Int_232-2

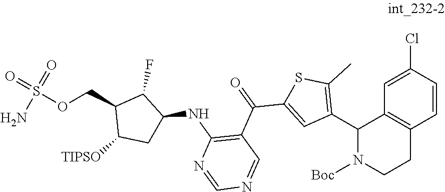

Int_232-1 (0.40 g, 517 μmol) was dissolved in DMF (6 mL), and int_1-17 (0.24 g, 2.07 mmol) was added to the reaction solution at room temperature. The reaction solution was allowed to react at room temperature for 1 h. LC-MS monitoring showed the reaction was completed. Ice water (10 mL) was added to the reaction solution. The aqueous phase was extracted with ethyl acetate (10 mL×3), and the organic phase was dried over anhydrous sodium sulfate. The organic phase was concentrated under reduced pressure to give a crude product. The crude product was purified by preparative column chromatography to give a solid (430 mg, yield: 97.7%).

MS (ESI): 852 [M+H]⁺.

### Step 3: Synthesis of Compound Int_232-3

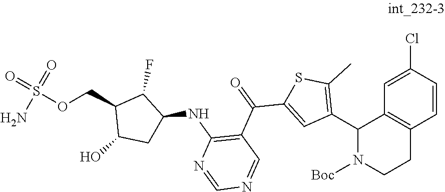 [SMILES: Cc1sc(C(=O)c2cncnc2N[C@H]2C[C@H](O)[C@@H](COS(N)(=O)=O)[C@@H]2F)cc1C1c2cc(Cl)ccc2CCN1C(=O)OC(C)(C)C]

Int_232-2 (0.62 g, 727 μmol) was dissolved in THF (8 mL), and TBAF (1.00 M, 1.45 mL) was added to the reaction solution at room temperature. The reaction solution was allowed to react at room temperature for 1 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography (ISCO®; Silica Flash Column, Eluent of 0-100% Ethyl acetate/Petroleum ether gradient) to give a solid (480 mg, yield: 94.8%).

MS (ESI): 696 [M+H]⁺.

### Step 4: Synthesis of Compound 232

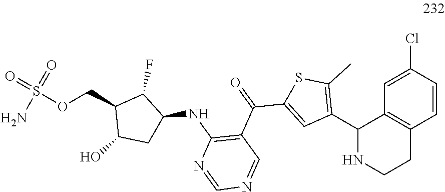 [SMILES: Cc1sc(C(=O)c2cncnc2N[C@H]2C[C@H](O)[C@@H](COS(N)(=O)=O)[C@@H]2F)cc1C1NCCc2ccc(Cl)cc21]

Int_232-3 (0.26 g, 373 μmol) was added to TFA (1 mL) at room temperature, and the reaction solution was allowed to react at room temperature for 10 min. LC-MS monitoring showed the reaction was completed. A saturated aqueous sodium bicarbonate solution was added to the reaction solution to adjust the pH to 8, and then the reaction solution was purified by preparative HPLC (column: Boston Prime C18 150×30 mm×5 μm; mobile phase: [water (NH₃H2O+NH₄HCO3)-ACN]; gradient: 25%-65% B over 9 min) to give a solid (200 mg, yield: 90.9%).

MS (ESI): 596 [M+H]⁺.

### Example 18. Synthesis of Compound 233 and Compound 234

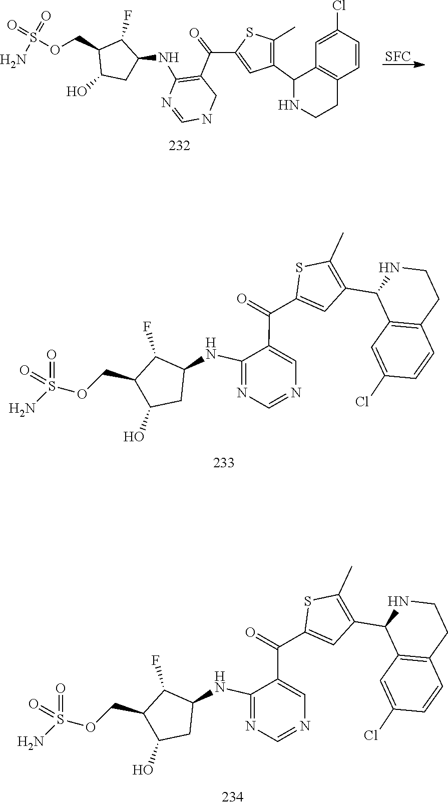

Compound 232 (100 mg, 167 μmol) was subjected to SFC chiral resolution (column: DAICEL CHIRALPAK IG (250 mm×30 mm, 10 μm); mobile phase: [CO₂-EtOH (0.1% NH₃H₂O)]; 60% B, isocratic elution mode) to give compound 233 (26 mg) and compound 234 (26 mg).

Compound 233: ¹H NMR (400 MHz, DMSO-d6) δ=8.64-8.55 (m, 2H), 8.35 (d, J=8.3 Hz, 1H), 7.54 (s, 2H), 7.35 (s, 1H), 7.18 (s, 2H), 6.67 (s, 1H), 5.20 (d, J=4.6 Hz, 1H), 5.17-5.13 (m, 1H), 5.05 (br dd, J=8.8, 19.1 Hz, 1H), 5.00-4.79 (m, 1H), 4.21-4.08 (m, 2H), 3.93 (br s, 1H), 3.77 (s, 1H), 3.15 (br s, 1H), 2.95-2.85 (m, 2H), 2.70 (br d, J=16.6 Hz, 1H), 2.50-2.42 (m, 3H), 2.31-2.20 (m, 1H), 1.98 (br d, J=10.4 Hz, 1H), 1.93-1.80 (m, 1H), 1.24 (s, 1H), 1.05 (d, J=6.6 Hz, 1H), 0.86 (s, 1H); MS (ESI): 596 [M+H]⁺. Analytical SFC retention time: 1.960 min (Instrument: Waters UPCC with PDA Detector; Column: Chiralpak IG-3 50x4.6 mm I.D., 3 um; Mobile phase: A: CO₂, B: Ethanol (0.05% DEA); Isocratic: 40% B; Flow rate: 4 mL/min; Column temp: 35° C.; ABPR: 1500 psi).

Compound 234: ¹H NMR (400 MHz, DMSO-d6) δ=8.63-8.54 (m, 2H), 8.32 (d, J=8.3 Hz, 1H), 7.54 (s, 2H), 7.32 (s, 1H), 7.18 (s, 2H), 6.66 (s, 1H), 5.19 (d, J=4.5 Hz, 1H), 5.15 (s, 1H), 5.12-5.00 (m, 1H), 4.99-4.78 (m, 1H), 4.23-4.05 (m, 2H), 3.91 (br d, J=4.0 Hz, 1H), 3.14 (br d, J=3.4 Hz, 1H), 2.97-2.84 (m, 2H), 2.70 (br d, J=14.9 Hz, 1H), 2.50 (br s, 3H), 2.32-2.20 (m, 1H), 2.01-1.91 (m, 1H), 1.88-1.76 (m, 1H); MS (ESI): 596 [M+H]⁺.

Analytical SFC retention time: 0.712 min (Instrument: Waters UPCC with PDA Detector; Column: Chiralpak IG-3 50×4.6 mm I.D., 3 μm; Mobile phase: A: CO₂, B: Ethanol (0.05% DEA); Isocratic: 40% B; Flow rate: 4 mL/min; Column temp: 35° C.; ABPR: 1500 psi).

### Example 19. Synthesis of Compound 300

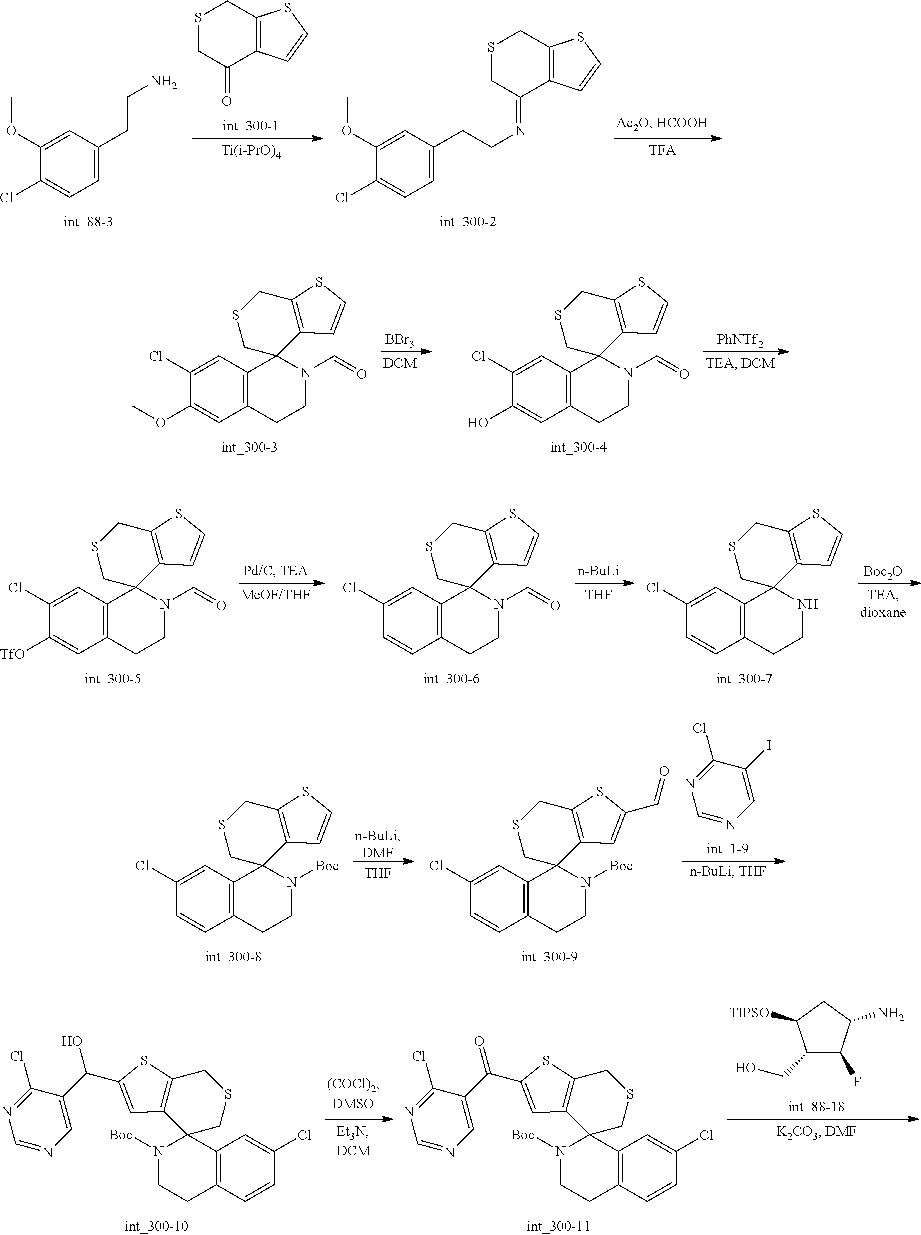

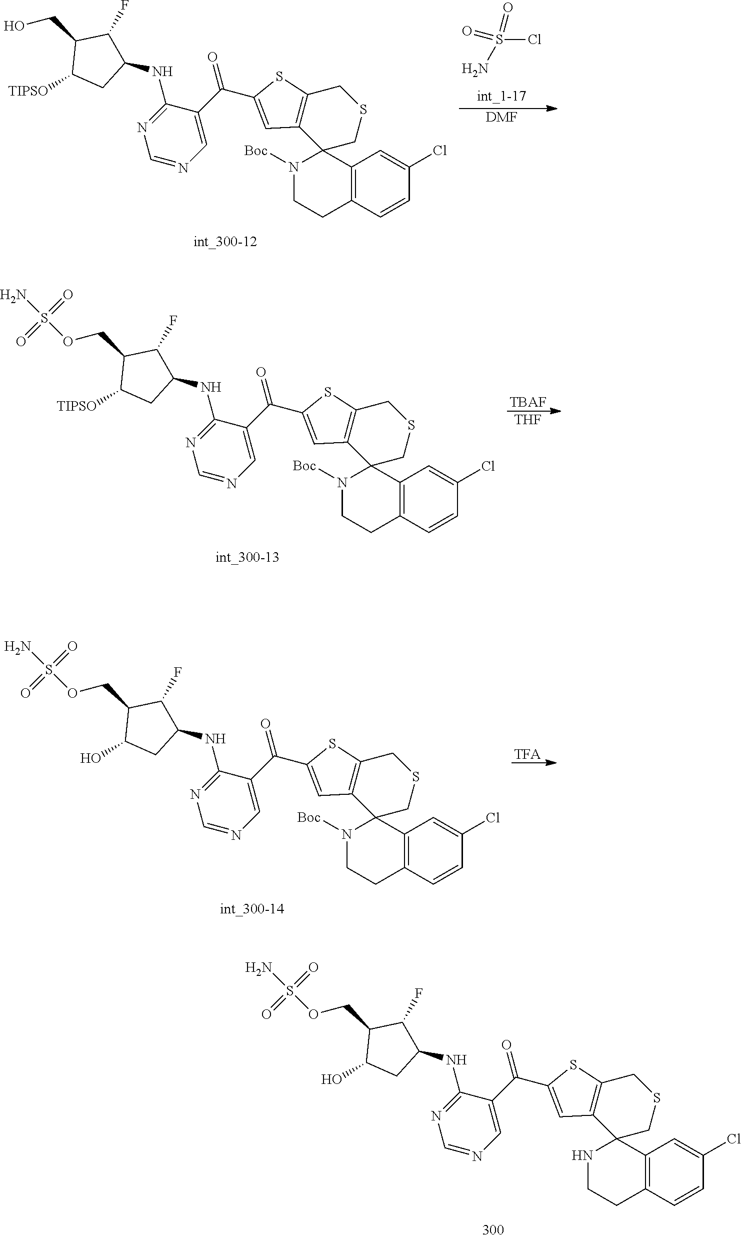

### Step 1: Synthesis of Compound Int 300-2

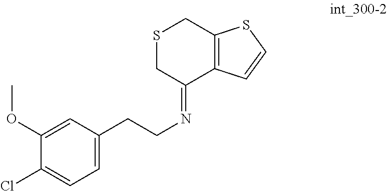 [SMILES: COc1cc(CC/N=C2\CSCc3sccc32)ccc1Cl]

Int_88-3 (9.00 g, 48.48 mmol) and int_300-1 (9.07 g, 53.33 mmol) were dissolved in Ti(i-PrO)₄ (170 mL). The mixed solution was purged with nitrogen three times, heated to 80° C., and stirred for 2 h. LC-MS monitoring showed the reaction was completed. The reaction solution was cooled to room temperature, which was directly used in the next step.

ESI-MS m/z: 338 [M+H]⁺.

### Step 2: Synthesis of Compound Int_300-3

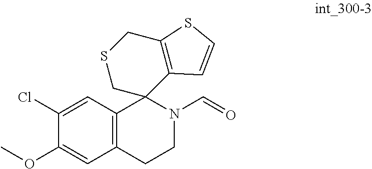 [SMILES: COc1cc2c(cc1Cl)C1(CSCc3sccc31)N(C=O)CC2]

HCOOH (200 mL) was slowly and dropwise added to Ac₂O (500 mL) at −10° C., and after the addition was completed, the reaction solution was allowed to react at 20° C. for 0.5 h. Then, the int_300-2 solution obtained in step 3 was cooled to −10° C., and the mixed solution of HOOCH and Ac₂O described above was slowly and dropwise added to the int 300-2 solution. The temperature was kept at −10° C. during the dropwise addition. After the dropwise addition was completed, the reaction solution was warmed to 70° C. and allowed to react for 3 h. LC-MS monitoring showed the reaction was completed. The reaction solution was cooled to room temperature. The organic phase was concentrated under reduced pressure to give a crude product, and the crude product was adjusted to pH>7 with a saturated aqueous sodium bicarbonate solution. The aqueous phase was extracted with ethyl acetate (500 mL×3), and the organic phase was dried over anhydrous sodium sulfate. The organic phase was concentrated under reduced pressure to give a crude product. The crude product was purified by preparative column chromatography (SiO₂, PE/EtOAc=1/1) to give a solid (2.3 g, yield: 11.7%).

¹H NMR (400 MHz, CHLOROFORM-d) δ=7.91 (s, 1H), 7.06 (d, J=5.3 Hz, 1H), 6.73 (s, 1H), 6.70 (s, 1H), 6.25 (d, J=5.3 Hz, 1H), 4.80-4.69 (m, 1H), 4.18 (d, J=16.3 Hz, 1H), 3.90 (s, 3H), 3.71 (dd, J=16.3, 1.5 Hz, 1H), 3.27-3.11 (m, 2H), 3.06-2.97 (m, 2H), 2.87-2.75 (m, 1H).

ESI-MS m/z: 366 [M+H]⁺.

### Step 3: Synthesis of Compound Int_300-4

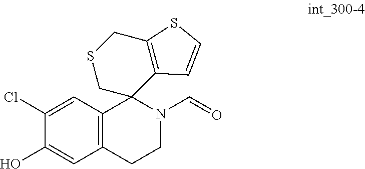 [SMILES: O=CN1CCc2cc(O)c(Cl)cc2C12CSCc1sccc12]

Int_300-3 (3.50 g, 9.5 mmol) was dissolved in dichloromethane (30 mL), and the mixed solution was purged with nitrogen three times. The reaction solution was cooled to 0° C., and BBr₃ (11.88 g, 47.43 mmol) was slowly and dropwise added to the reaction solution. The reaction solution was warmed to room temperature and allowed to react for 1 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product (3 g, yield: 90.9%), which was directly used in the next step.

ESI-MS m/z: 352 [M+H]⁺.

### Step 4: Synthesis of Compound Int_300-5

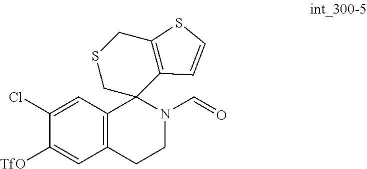 [SMILES: O=CN1CCc2cc(OS(=O)(=O)C(F)(F)F)c(Cl)cc2C12CSCc1sccc12]

Int_300-4 (3 g, 8.5 mmol), PhNTf₂ (5.46 g, 15.3 mmol), and TEA (2.15 g, 21.25 mmol, 2.92 mL) were dissolved in dichloromethane (30 mL). The reaction solution was purged with nitrogen three times, and allowed to react at room temperature for 16 h. LC-MS monitoring showed the reaction was completed. The reaction solution was poured slowly into 300 mL of ice water. The aqueous phase was extracted with ethyl acetate (300 mL×3), and the organic phase was dried over anhydrous sodium sulfate. The organic phase was concentrated under reduced pressure to give a crude product. The crude product was purified by preparative column chromatography (SiO₂, PE/EtOAc=1/1) to give a solid (2.9 g, yield: 70.7%).

ESI-MS m/z: 484 [M+H]⁺.

### Step 5: Synthesis of Compound Int_300-6

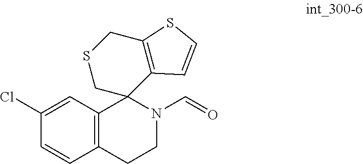 [SMILES: O=CN1CCc2ccc(Cl)cc2C12CSCc1sccc12]

Int_300-5 (2.9 g, 5.99 mmol), Pd/C (1.2 g, 10% purity), and TEA (2.42 g, 23.96 mmol, 3.33 mL) were dissolved in a mixed solvent of methanol (25 mL) and tetrahydrofuran (10 mL). The reaction solution was purged with hydrogen three times, and allowed to react at room temperature for 16 h. LC-MS monitoring showed the reaction was completed. The reaction solution was filtered to give a filtrate, and the filtrate was concentrated under reduced pressure to give a crude product. The crude product was purified by preparative column chromatography (SiO₂, PE/EtOAc=1/1) to give a solid (0.85 g, yield: 42.5%).

ESI-MS m/z: 336 [M+H]⁺.

### Step 6: Synthesis of Compound Int_300-7

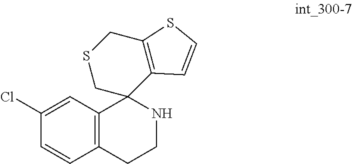 [SMILES: Clc1ccc2c(c1)C1(CSCc3sccc31)NCC2]

Int_300-6 (0.85 g, 2.53 mmol) was dissolved in THF (10 mL), and the mixture was cooled to −30° C. n-BuLi (2.5 M, 3.04 mL) was slowly and dropwise added to the reaction solution under nitrogen atmosphere, and the reaction solution was allowed to react at −30° C. for 2 h. Subsequently, the reaction solution was warmed to room temperature and allowed to react for 1 h. LC-MS monitoring showed the reaction was completed. A saturated aqueous ammonium chloride solution (10 mL) was added to the reaction solution. The aqueous phase was extracted with ethyl acetate (30 mL×3), and the organic phase was dried over anhydrous sodium sulfate. The organic phase was concentrated under reduced pressure to give a crude product (0.71 g, yield: 91.2%), which was directly used in the next step.

¹H NMR (400 MHz, DMSO-d6) δ=7.16 (d, J=1.4 Hz, 2H), 7.14 (d, J=5.3 Hz, 1H), 6.85 (t, J=1.3 Hz, 1H), 6.35 (d, J=5.3 Hz, 1H), 4.16 (d, J=16.1 Hz, 1H), 3.69 (dd, J=16.0, 1.3 Hz, 1H), 3.25-3.11 (m, 2H), 2.98 (m, 2H), 2.88 (m, 1H), 2.74 (br, 1H), 2.68 (dt, J=16.0, 3.5 Hz, 1H).

ESI-MS m/z: 308 [M+H]⁺.

### Step 7: Synthesis of Compound Int_300-8

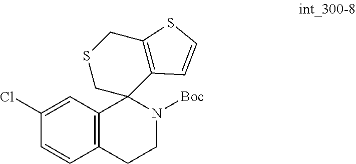 [SMILES: CC(C)(C)OC(=O)N1CCc2ccc(Cl)cc2C12CSCc1sccc12]

Int_300-7 (0.71 g, 2.30 mmol) and (Boc)₂O (1 g, 4.60 mmol) were dissolved in 1,4-dioxane (10 mL), and TEA (581.8 mg, 5.75 mmol, 801.4 μL) was added to the reaction solution at room temperature. The reaction solution was warmed to 80° C. and allowed to react for 16 h. LC-MS monitoring showed the reaction was completed. After the reaction solution was cooled to room temperature, the reaction solution was concentrated under reduced pressure to give a crude product. The crude product was purified by preparative column chromatography (SiO₂, PE/EtOAc=10/1) to give a solid (0.65 g, yield: 69.2%).

¹H NMR (400 MHz, DMSO-d6) δ=7.29 (d, J=5.3 Hz, 1H), 7.24 (d, J=1.4 Hz, 2H), 6.73 (t, J=1.3 Hz, 1H), 6.53 (d, J=5.3 Hz, 1H), 4.23 (dt, J=12.1, 3.7 Hz, 1H), 3.96 (d, J=16.1 Hz, 1H), 3.90 (d, J=13.5 Hz, 1H), 3.81 (dd, J=16.1, 1.5 Hz, 1H), 3.15 (td, J=12.0, 3.2 Hz, 1H), 2.98 (ddd, J=15.8, 11.7, 4.1 Hz, 1H), 2.89 (dt, J=15.5, 3.3 Hz, 1H), 2.52 (d, J=1.6 Hz, 1H), 1.15 (s, 9H).

ESI-MS m/z: 408 [M+H]⁺.

### Step 8: Synthesis of Compound Int_300-9

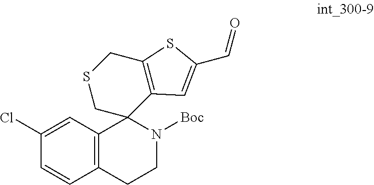 [SMILES: CC(C)(C)OC(=O)N1CCc2ccc(Cl)cc2C12CSCc1sc(C=O)cc12]

Int_300-8 (1.5 g, 3.67 mmol) was dissolved in THF (20 mL), and the mixture was cooled to −75° C. n-BuLi (2.5 M, 4.43 mL) was slowly and dropwise added to the reaction solution under nitrogen atmosphere, and the reaction solution was allowed to react at −75° C. for 1 h. Then, DMF (809 mg, 11.08 mmol) was dropwise added to the reaction solution at −75° C., and after the dropwise addition was completed, the reaction solution was allowed to react at −75° C. for 1 h. LC-MS monitoring showed the reaction was completed. A saturated ammonium chloride solution (100 mL) was slowly added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (1.25 g, yield: 78.1%).

MS (ESI): 436 [M+H]⁺.

### Step 9: Synthesis of Compound Int_300-10

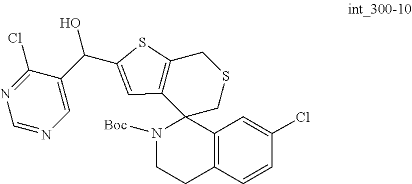 [SMILES: CC(C)(C)OC(=O)N1CCc2ccc(Cl)cc2C12CSCc1sc(C(O)c3cncnc3Cl)cc12]

Int_1-9 (4.8 g, 19.92 mmol) was dissolved in THF (50 mL), and the mixture was cooled to −75° C. n-BuLi (2.5 M, 16 mL) was slowly and dropwise added to the reaction solution under nitrogen atmosphere, and the reaction solution was allowed to react at −75° C. for 1 h. Then, a solution of int_300-9 (1.45 g, 3.33 mmol) in THF (25 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (200 mL) was slowly added to the reaction solution. The aqueous phase was extracted with ethyl acetate (200 mL×3), and the organic phase was dried over anhydrous sodium sulfate. The organic phase was concentrated under reduced pressure to give a crude product. The crude product was purified by preparative column chromatography to give a solid (1.51 g, yield: 82.5%).

MS (ESI): 550 [M+H]⁺.

### Step 10: Synthesis of Compound Int_300-11

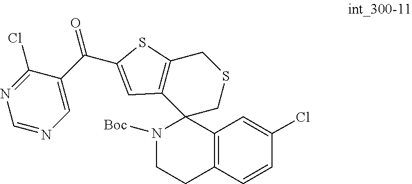 [SMILES: CC(C)(C)OC(=O)N1CCc2ccc(Cl)cc2C12CSCc1sc(C(=O)c3cncnc3Cl)cc12]

Oxalyl chloride (413.7 mg, 3.26 mmol, 279 μL) was dissolved in dichloromethane (20 mL), and dimethyl sulfoxide (254.7 mg, 3.26 mmol, 231 μL) was slowly and dropwise added to the reaction solution at −78° C. The reaction solution was allowed to react at −78° C. for 0.5 h. A solution of int_300-10 (1.5 g, 2.72 mmol) in DCM (10 mL) was dropwise added to the reaction solution, and the reaction solution was allowed to react at −78° C. for another 0.5 h. Triethylamine (1.65 g, 16.32 mmol) was dropwise added to the reaction solution, and the reaction solution was allowed to react at −78° C. for another 0.5 h and then slowly warmed to room temperature. LC-MS monitoring showed the reaction was completed. A saturated aqueous sodium bicarbonate solution was slowly added to the reaction solution to adjust the pH to about 8. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (1.38 g, yield: 92.6%).

MS (ESI): 548 [M+H]⁺.

### Step 11: Synthesis of Compound Int_300-12

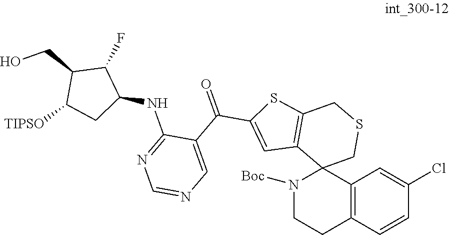

Int_300-11 (1.097 g, 2.0 mmol) and int_1-17 (672 mg, 2.2 mmol) were dissolved in DMF (10 mL), and K₂CO₃ (829.2 mg, 6.0 mmol) was added to the reaction solution at room temperature. The reaction solution was allowed to react at room temperature for 3 h. LC-MS monitoring showed the reaction was completed. Ice water (100 mL) was added to the reaction solution. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a product (1.3 g, yield: 79.7%).

MS (ESI): 817 [M+H]⁺.

### Step 12: Synthesis of Compound Int_300-13

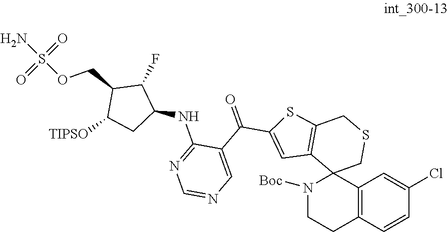

Int_300-12 (1.38 g, 1.7 mmol) was dissolved in DMF (30 mL), and int_1-17 (395.9 mg, 3.42 mmol) was added to the reaction solution at room temperature. The reaction solution was allowed to react at room temperature for 1 h. LC-MS monitoring showed the reaction was completed. Ice water (50 mL) was added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (1.2 g, yield: 78.9%).

MS (ESI): 896 [M+H]⁺.

### Step 13: Synthesis of Compound Int 300-14

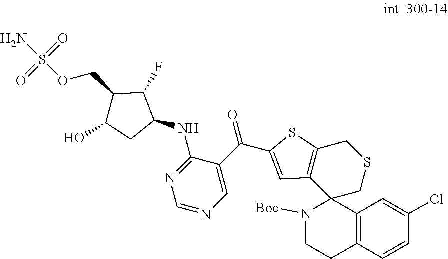 [SMILES: CC(C)(C)OC(=O)N1CCc2ccc(Cl)cc2C12CSCc1sc(C(=O)c3cncnc3N[C@H]3C[C@H](O)[C@@H](COS(N)(=O)=O)[C@@H]3F)cc12]

Int_300-13 (1.61 g, 1.8 mmol) was dissolved in THF (20 mL), and TBAF (1 M, 3.6 mL) was added to the reaction solution at room temperature. The reaction solution was allowed to react at room temperature for 4 h. LC-MS monitoring showed the reaction was completed. Water (50 mL) was added to the reaction solution. The aqueous phase was extracted with ethyl acetate (50 mL×3), and the organic phase was dried over anhydrous sodium sulfate. The organic phase was concentrated under reduced pressure to give a crude product. The crude product was purified by preparative column chromatography to give a solid (1.1 g, yield: 82.7%).

MS (ESI): 740 [M+H]⁺.

### Step 14: Synthesis of Compound 300

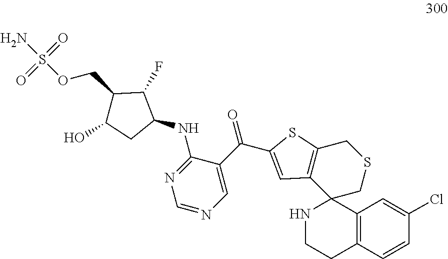 [SMILES: NS(=O)(=O)OC[C@H]1[C@H](F)[C@@H](Nc2ncncc2C(=O)c2cc3c(s2)CSCC32NCCc3ccc(Cl)cc32)C[C@@H]1O]

Int_300-14 (1.11 g, 1.5 mmol) was dissolved in DCM (7 mL) at room temperature, and the mixture was added into TFA (7.8 mL). The reaction solution was allowed to react at room temperature for 5 min. LC-MS monitoring showed the reaction was completed. The reaction solution was cooled to 0° C. A saturated aqueous sodium bicarbonate solution was added to the reaction solution to adjust the pH to 8. The aqueous phase was extracted with ethyl acetate (50 mL×3), and the organic phase was dried over anhydrous sodium sulfate. The organic phase was concentrated under reduced pressure to give a crude product. The crude product was purified by preparative HPLC to give a solid (816 mg, yield: 85.0%).

MS (ESI): 640 [M+H]⁺.

### Example 20. Synthesis of Compound 301 and Compound 302

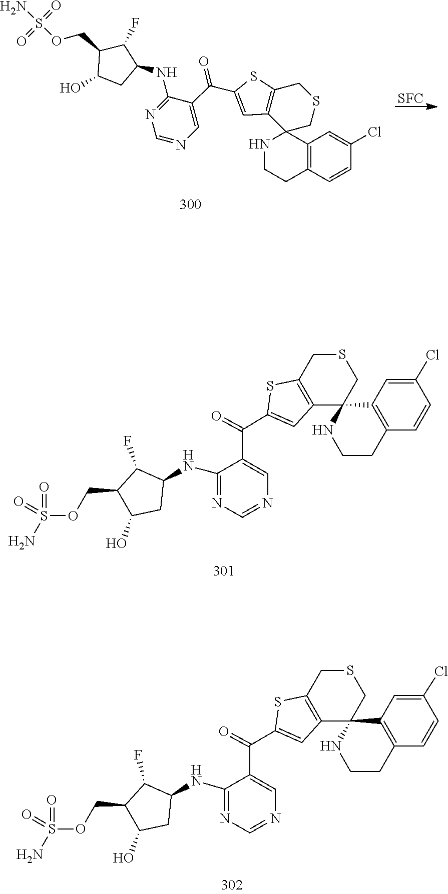 [SMILES: NS(=O)(=O)OC[C@H]1[C@H](F)[C@@H](Nc2ncncc2C(=O)c2cc3c(s2)CSCC32NCCc3ccc(Cl)cc32)C[C@@H]1O ; "SFC" ; NS(=O)(=O)OC[C@H]1[C@H](F)[C@@H](Nc2ncncc2C(=O)c2cc3c(s2)CSC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)CSC[C@]32NCCc3ccc(Cl)cc32)C[C@@H]1O]

Compound 300 (150 mg, 0.234 mmol) was subjected to SFC chiral resolution (column: DAICEL CHIRALPAK IG (250 mm×30 mm, 10 μm); mobile phase: [CO₂-EtOH (0.1% NH₃H₂O)]) to give compound 301 (56 mg) and compound 302 (58 mg).

Compound 301: ¹H NMR (400 MHz, DMSO-d6) δ 8.55 (s, 1H), 8.37 (d, J=3.0 Hz, 1H), 8.36-8.26 (m, 1H), 7.51 (s, 2H), 7.21-7.12 (m, 2H), 6.96 (t, J=2.6 Hz, 2H), 5.17 (dd, J=4.7, 2.2 Hz, 1H), 5.02 (m, 1H), 4.86 (dtd, J=54.2, 6.7, 3.1 Hz, 1H), 4.27 (d, J=16.8 Hz, 1H), 4.20-4.05 (m, 2H), 3.95-3.81 (m, 2H), 3.31-3.16 (m, 2H), 3.08-2.81 (m, 4H), 2.67 (d, J=15.5 Hz, 1H), 2.24 (m, 1H), 1.99-1.88 (m, 1H), 1.87-1.72 (m, 1H); MS (ESI): 640 [M+H]⁺.

Compound 302: ¹H NMR (400 MHz, DMSO-d6) δ 8.54 (s, 1H), 8.36 (d, J=3.0 Hz, 1H), 8.35-8.26 (m, 1H), 7.50 (s, 2H), 7.21-7.11 (m, 2H), 6.95 (t, J=2.6 Hz, 2H), 5.16 (dd, J=4.7, 2.2 Hz, 1H), 5.01 (dt, J=18.8, 8.3 Hz, 1H), 4.86 (dtd, J=54.2, 6.7, 3.1 Hz, 1H), 4.27 (d, J=16.8 Hz, 1H), 4.19-4.04 (m, 2H), 3.93-3.81 (m, 2H), 3.29-3.15 (m, 2H), 3.07-2.81 (m, 4H), 2.66 (d, J=15.5 Hz, 1H), 2.23 (dq, J=22.5, 5.8 Hz, 1H), 1.99-1.87 (m, 1H), 1.87-1.72 (m, 1H); MS (ESI): 640 [M+H]⁺.

### Example 21. Synthesis of Compound 576

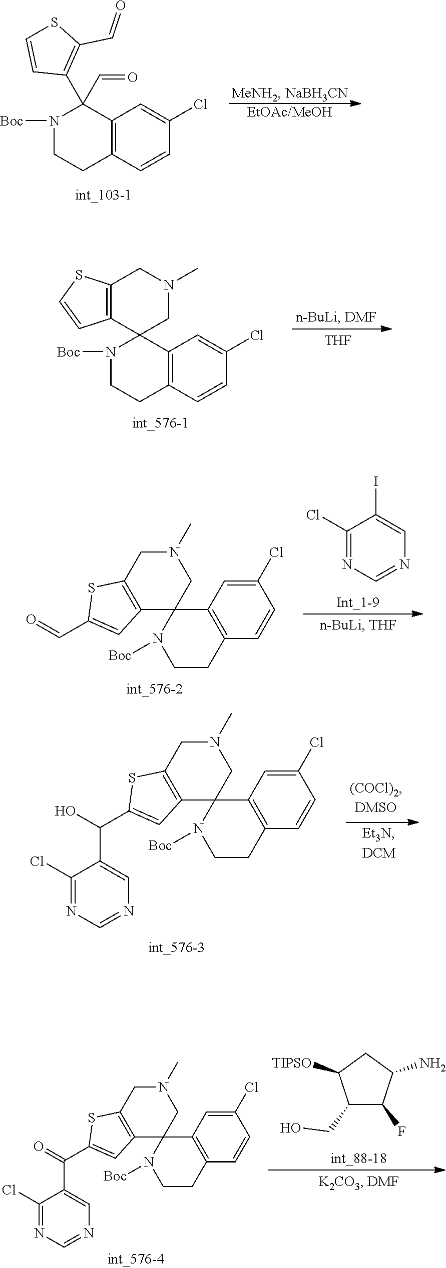

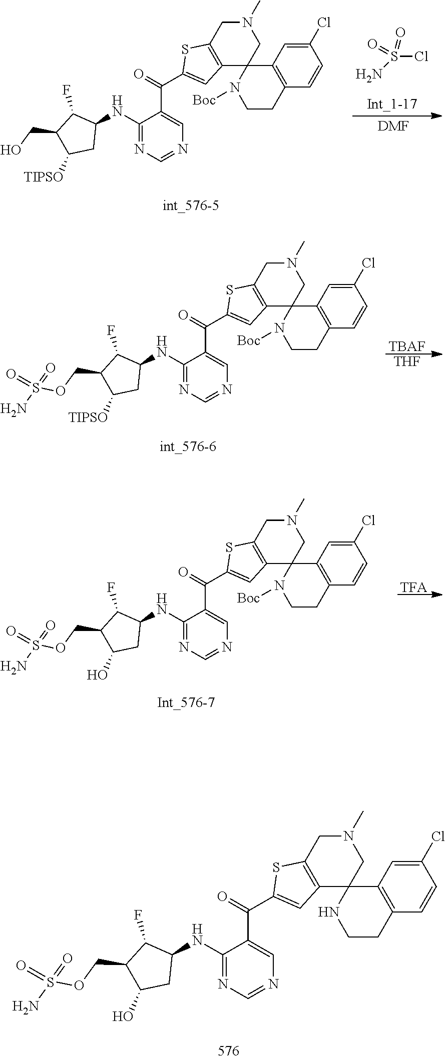

### Step 1: Synthesis of Compound Int_576-1

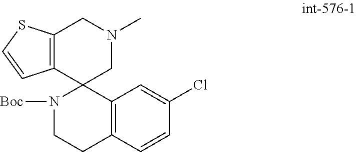 [SMILES: CN1Cc2sccc2C2(C1)c1cc(Cl)ccc1CCN2C(=O)OC(C)(C)C]

Int_103-1 (2.5 g, 6.15 mmol) was dissolved in a mixed solvent of ethyl acetate (60 mL) and methanol (15 mL), and methylamine (573 mg, 18.45 mmol, 1.88 mL, 40% in MeOH) and sodium cyanoborohydride (1.53 g, 24.63 mmol) were added to the reaction solution. The reaction solution was allowed to react at room temperature for 16 h. LC-MS monitoring showed the reaction was completed. The reaction solution was poured slowly into 100 mL of ice water. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (2.1 g, yield: 84.3%).

ESI-MS m/z: 405 [M+H]⁺.

### Step 2: Synthesis of Compound Int_576-2

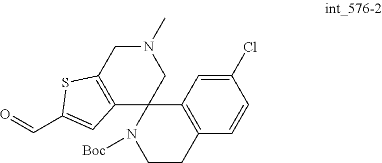 [SMILES: CN1Cc2sc(C=O)cc2C2(C1)c1cc(Cl)ccc1CCN2C(=O)OC(C)(C)C]

Int_576-1 (283.4 mg, 0.7 mmol) was dissolved in THF (10 mL), and the mixture was cooled to −70° C. n-BuLi (2.5 M, 0.83 mL) was slowly and dropwise added to the reaction solution under nitrogen atmosphere, and the reaction solution was allowed to react at −70° C. for 1 h. Then, DMF (153 mg, 2.1 mmol) was dropwise added to the reaction solution at −60° C., and after the dropwise addition was completed, the reaction solution was allowed to react at 70° C. for 1 h. LC-MS monitoring showed the reaction was completed. A saturated ammonium chloride solution (20 mL) was slowly added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography to give a solid (169 mg, yield: 55.7%).

MS (ESI): 433 [M+H]⁺.

### Step 3: Synthesis of Compound Int_576-3

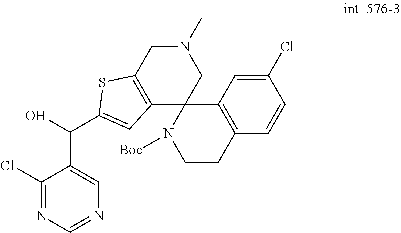 [SMILES: CN1Cc2sc(C(O)c3cncnc3Cl)cc2C2(C1)c1cc(Cl)ccc1CCN2C(=O)OC(C)(C)C]

Int_1-9 (577 mg, 2.4 mmol) was dissolved in THF (10 mL), and the mixture was cooled to −70° C. n-BuLi (2.5 M, 1.9 mL, 4.8 mmol) was slowly and dropwise added to the reaction solution under nitrogen atmosphere, and the reaction solution was allowed to react at −70° C. for 1 h. Then, a solution of int_576-2 (173.1 mg, 0.4 mmol) in THF (1 mL) was dropwise added to the reaction solution at −75° C., and after the dropwise addition was completed, the reaction solution was allowed to react at −75° C. for 1 h, and was then warmed to room temperature and allowed to react for 16 h. L C-MS monitoring showed the reaction was completed. A saturated ammonium chloride solution (20 mL) was slowly added to the reaction solution. The aqueous phase was extracted with ethyl acetate (20 mL×3), and the organic phase was dried over anhydrous sodium sulfate. The organic phase was concentrated under reduced pressure to give a crude product. The crude product was purified by preparative column chromatography to give a solid (162 mg, yield: 73.9%).

MS (ESI): 547 [M+H]⁺.

### Step 4: Synthesis of Compound Int_576-4

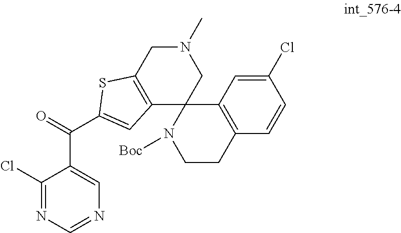 [SMILES: CN1Cc2sc(C(=O)c3cncnc3Cl)cc2C2(C1)c1cc(Cl)ccc1CCN2C(=O)OC(C)(C)C]

Oxalyl chloride (415 mg, 3.27 mmol, 280 μL) was dissolved in dichloromethane (20 mL), and dimethyl sulfoxide (254.75 mg, 3.27 mmol, 232 μL) was slowly and dropwise added to the reaction solution at −78° C. The reaction solution was allowed to react at −78° C. for 0.5 h. A solution of int_576-3 (1.5 g, 2.73 mmol) in DCM (10 mL) was dropwise added to the reaction solution, and the reaction solution was allowed to react at −78° C. for another 0.5 h. Triethylamine (1.65 g, 16.32 mmol) was dropwise added to the reaction solution, and the reaction solution was allowed to react at −78° C. for another 0.5 h and then slowly warmed to room temperature. LC-MS monitoring showed the reaction was completed. A saturated aqueous sodium bicarbonate solution was slowly added to the reaction solution to adjust the pH to about 8. The aqueous phase was extracted with ethyl acetate (100 mL×3), and the organic phase was dried over anhydrous sodium sulfate. The organic phase was concentrated under reduced pressure to give a crude product. The crude product was purified by preparative column chromatography to give a solid (0.56 g, yield: 37.8%).

MS (ESI): 545 [M+H]⁺.

### Step 5: Synthesis of Compound Int_576-5

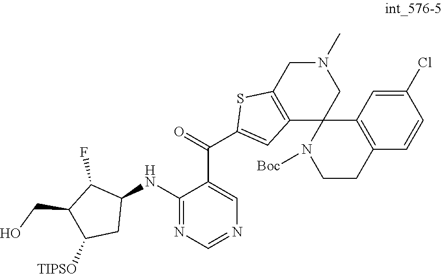

Int_576-4 (272.7 mg, 0.5 mmol) and int_88-18 (183.3 mg, 0.6 mmol) were dissolved in DMF (10 mL), and K₂CO₃ (207.3 mg, 1.5 mmol) was added to the reaction solution at room temperature. The reaction solution was allowed to react at room temperature for 16 h. LC-MS monitoring showed the reaction was completed. Ice water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a product (288 mg, yield: 70.7%).

MS (ESI): 814 [M+H]⁺.

### Step 6: Synthesis of Compound Int_576-6

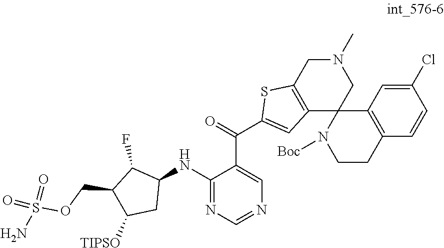

Int_576-5 (285 mg, 0.35 mmol) was dissolved in DMF (4 mL), and int_1-17 (80.8 mg, 0.7 mmol) was added to the reaction solution at room temperature. The reaction solution was allowed to react at room temperature for 1 h. LC-MS monitoring showed the reaction was completed. Ice water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a solid (236 mg, yield: 75.6%).

MS (ESI): 893 [M+H]⁺.

### Step 7: Synthesis of Compound Int 576-7

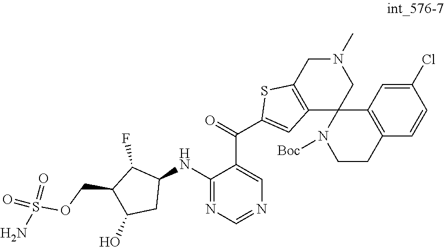 [SMILES: CN1Cc2sc(C(=O)c3cncnc3N[C@H]3C[C@H](O)[C@@H](COS(N)(=O)=O)[C@@H]3F)cc2C2(C1)c1cc(Cl)ccc1CCN2C(=O)OC(C)(C)C]

Int_576-6 (259 mg, 0.29 mmol) was dissolved in TIF (4 mL), and TBAF (1 M, 0.58 mL, 0.58 mmol) was added to the reaction solution at room temperature. The reaction solution was allowed to react at room temperature for 16 h. LC-MS monitoring showed the reaction was completed. Water (30 mL) was added to the reaction solution. The aqueous phase was extracted with ethyl acetate (30 mL×3), and the organic phase was dried over anhydrous sodium sulfate. The organic phase was concentrated under reduced pressure to give a crude product. The crude product was purified by preparative column chromatography to give a solid (180 mg, yield: 84.5%).

MS (ESI): 737 [M+H]⁺.

### Step 8: Synthesis of Compound 576

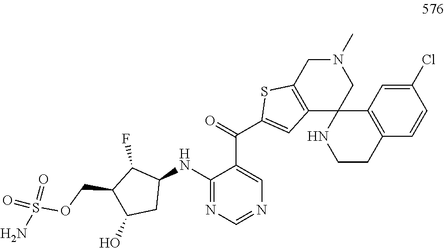 [SMILES: CN1Cc2sc(C(=O)c3cncnc3N[C@H]3C[C@H](O)[C@@H](COS(N)(=O)=O)[C@@H]3F)cc2C2(C1)NCCc1ccc(Cl)cc12]

Int_576-7 (184 mg, 0.25 mmol) was dissolved in dichloromethane (6 mL), and TFA (1.5 mL) was added to the reaction solution at room temperature. The reaction solution was allowed to react at room temperature for 10 min. LC-MS monitoring showed the reaction was completed. Ammonia water (1 mL) and water (10 mL) were added to the reaction solution. The aqueous phase was extracted with dichloromethane (10 mL×3), and the organic phase was dried over anhydrous sodium sulfate. The organic phase was concentrated under reduced pressure to give a crude product. The crude product was purified by preparative HPLC to give a solid (97 mg, yield: 61.0%).

MS (ESI): 637 [M+H]⁺.

### Example 22. Synthesis of Compound 577 and Compound 578

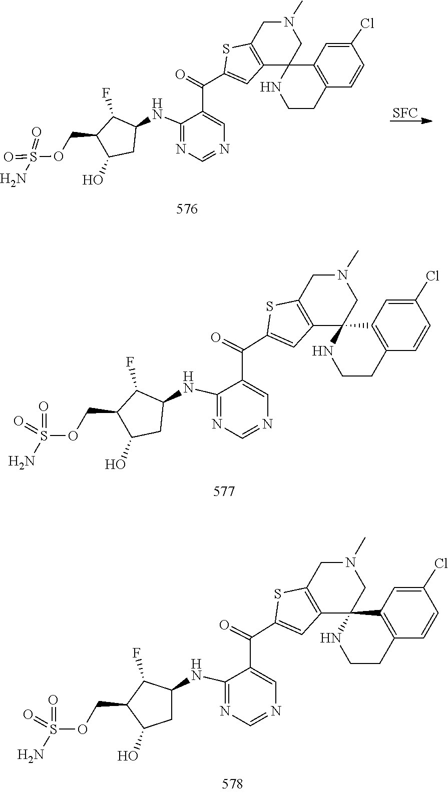

Compound 576 (85 mg, 0.133 mmol) was subjected to SFC chiral resolution (column: Phenomenex-Cellulose-2 (250 mm×30 mm, 10 μm)); mobile phase: [CO₂-MeOH (0.1% NH₃H₂O)]; B %: 45%, isocratic elution mode) to give compound 577 (21 mg) and compound 578 (25 mg).

Compound 577: ¹H NMR (400 MHz, DMSO-d6) δ=8.57 (s, 1H), 8.42 (d, J=5.2 Hz, 1H), 8.29 (dd, J=19.5, 8.2 Hz, 1H), 7.52 (s, 2H), 7.21-7.10 (m, 2H), 7.09 (d, J=3.6 Hz, 1H), 6.90 (t, J=2.4 Hz, 1H), 5.19 (dd, J=4.6, 3.6 Hz, 1H), 5.02 (dt, J=18.5, 8.5 Hz, 1H), 4.87 (ddd, J=54.3, 7.6, 6.1 Hz, 1H), 4.21-4.05 (m, 2H), 3.91 (s, 1H), 3.86-3.63 (m, 2H), 3.12-2.79 (m, 5H), 2.69 (m, 2H), 2.45-2.34 (m, 3H), 2.24 (m, 1H), 2.03-1.87 (m, 1H), 1.87-1.72 (m, 1H); MS (ESI): 637 [M+H]⁺.

Compound 578: ¹H NMR (400 MHz, DMSO-d6) δ=8.55 (s, 1H), 8.40 (d, J=5.1 Hz, 1H), 8.28 (dd, J=19.5, 8.2 Hz, 1H), 7.50 (s, 2H), 7.19-7.09 (m, 2H), 7.06 (d, J=3.6 Hz, 1H), 6.88 (t, J=2.4 Hz, 1H), 5.17 (dd, J=4.6, 3.6 Hz, 1H), 5.00 (dt, J=18.5, 8.5 Hz, 1H), 4.85 (ddd, J=54.2, 7.6, 6.0 Hz, 1H), 4.19-4.03 (m, 2H), 3.89 (s, 1H), 3.85-3.63 (m, 2H), 3.10-2.78 (m, 5H), 2.67 (dt, J=10.9, 5.7 Hz, 2H), 2.43-2.33 (m, 3H), 2.22 (dt, J=22.5, 5.8 Hz, 1H), 2.01-1.86 (m, 1H), 1.86-1.71 (m, 1H); MS (ESI): 637 [M+H]⁺.

The target compounds 4-87, 94-102, 106-129, 133-161, 163-189, 193-231, 235-342, 345-575, and 579-1070 in Table 1 were obtained using the synthetic methods described above with different starting materials.

TABLE 1

| Com- | | MS |
|---|---|---|
| pound | Compound structure | (M + H)⁺ |
| 1 | 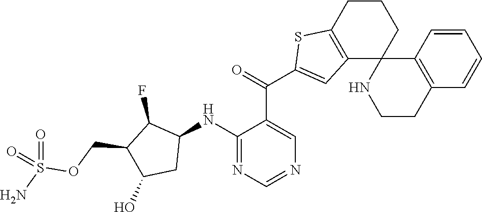 [SMILES: NS(=O)(=O)OC[C@H]1[C@@H](F)[C@@H](Nc2ncncc2C(=O)c2cc3c(s2)CCCC32NCCc3ccccc32)C[C@@H]1O] | 588 |
| 2 | 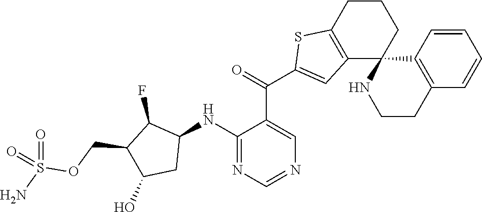 [SMILES: NS(=O)(=O)OC[C@H]1[C@@H](F)[C@@H](Nc2ncncc2C(=O)c2cc3c(s2)CCC[C@@]32NCCc3ccccc32)C[C@@H]1O] | 588 |
| 3 | 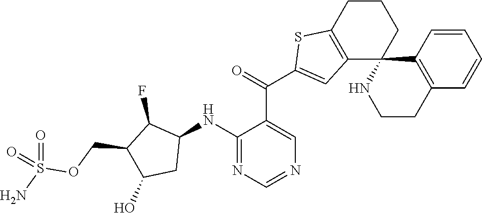 [SMILES: NS(=O)(=O)OC[C@H]1[C@@H](F)[C@@H](Nc2ncncc2C(=O)c2cc3c(s2)CCC[C@]32NCCc3ccccc32)C[C@@H]1O] | 588 |
| 4 | 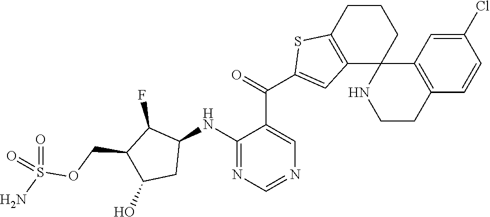 [SMILES: NS(=O)(=O)OC[C@H]1[C@@H](F)[C@@H](Nc2ncncc2C(=O)c2cc3c(s2)CCCC32NCCc3ccc(Cl)cc32)C[C@@H]1O] | 622 |
| 5 | 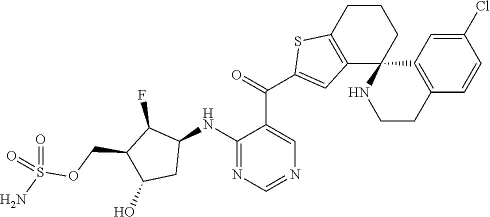 [SMILES: NS(=O)(=O)OC[C@H]1[C@@H](F)[C@@H](Nc2ncncc2C(=O)c2cc3c(s2)CCC[C@@]32NCCc3ccc(Cl)cc32)C[C@@H]1O] | 622 |
| 6 | 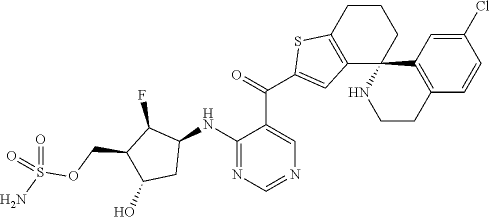 [SMILES: NS(=O)(=O)OC[C@H]1[C@@H](F)[C@@H](Nc2ncncc2C(=O)c2cc3c(s2)CCC[C@]32NCCc3ccc(Cl)cc32)C[C@@H]1O] | 622 |
| 7 | 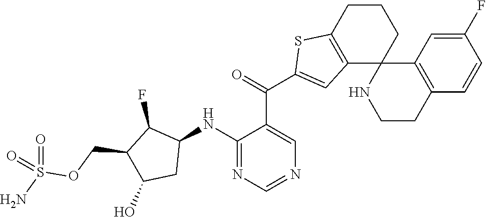 [SMILES: NS(=O)(=O)OC[C@H]1[C@@H](F)[C@@H](Nc2ncncc2C(=O)c2cc3c(s2)CCCC32NCCc3ccc(F)cc32)C[C@@H]1O] | 606 |
| 8 | 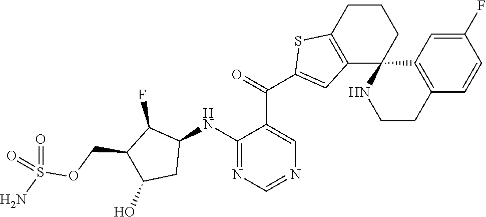 [SMILES: NS(=O)(=O)OC[C@H]1[C@@H](F)[C@@H](Nc2ncncc2C(=O)c2cc3c(s2)CCC[C@@]32NCCc3ccc(F)cc32)C[C@@H]1O] | 606 |
| 9 | 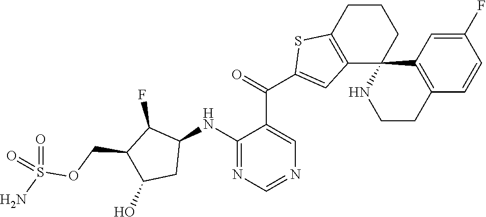 [SMILES: NS(=O)(=O)OC[C@H]1[C@@H](F)[C@@H](Nc2ncncc2C(=O)c2cc3c(s2)CCC[C@]32NCCc3ccc(F)cc32)C[C@@H]1O] | 606 |
| 10 | 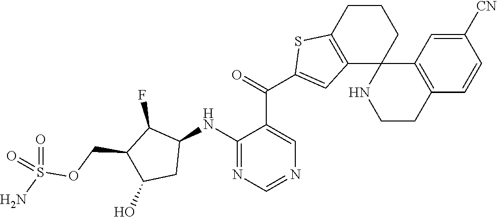 [SMILES: N#Cc1ccc2c(c1)C1(CCCc3sc(C(=O)c4cncnc4N[C@H]4C[C@H](O)[C@@H](COS(N)(=O)=O)[C@H]4F)cc31)NCC2] | 613 |
| 11 | 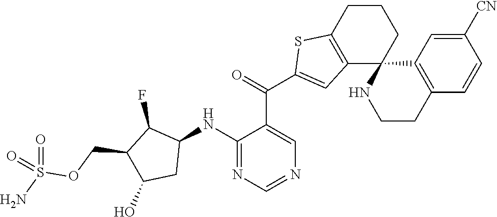 [SMILES: N#Cc1ccc2c(c1)[C@]1(CCCc3sc(C(=O)c4cncnc4N[C@H]4C[C@H](O)[C@@H](COS(N)(=O)=O)[C@H]4F)cc31)NCC2] | 613 |
| 12 | 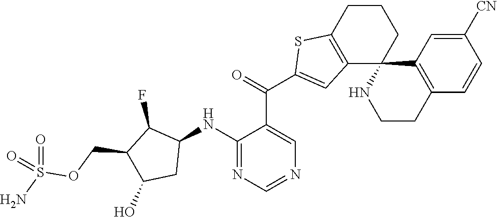 [SMILES: N#Cc1ccc2c(c1)[C@@]1(CCCc3sc(C(=O)c4cncnc4N[C@H]4C[C@H](O)[C@@H](COS(N)(=O)=O)[C@H]4F)cc31)NCC2] | 613 |
| 13 | 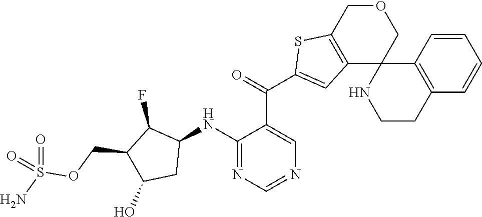 [SMILES: NS(=O)(=O)OC[C@H]1[C@@H](F)[C@@H](Nc2ncncc2C(=O)c2cc3c(s2)COCC32NCCc3ccccc32)C[C@@H]1O] | 590 |
| 14 | 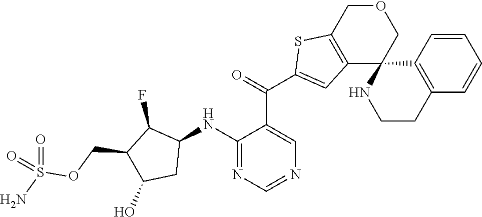 [SMILES: NS(=O)(=O)OC[C@H]1[C@@H](F)[C@@H](Nc2ncncc2C(=O)c2cc3c(s2)COC[C@@]32NCCc3ccccc32)C[C@@H]1O] | 590 |
| 15 | 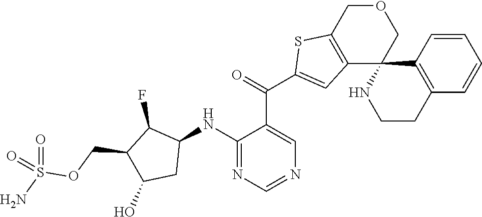 [SMILES: NS(=O)(=O)OC[C@H]1[C@@H](F)[C@@H](Nc2ncncc2C(=O)c2cc3c(s2)COC[C@]32NCCc3ccccc32)C[C@@H]1O] | 590 |
| 16 | 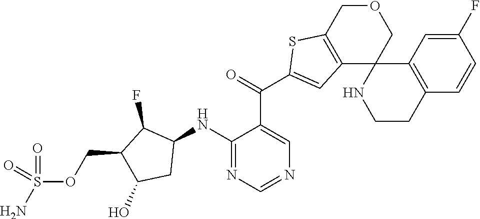 [SMILES: NS(=O)(=O)OC[C@H]1[C@@H](F)[C@@H](Nc2ncncc2C(=O)c2cc3c(s2)COCC32NCCc3ccc(F)cc32)C[C@@H]1O] | 608 |
| 17 | 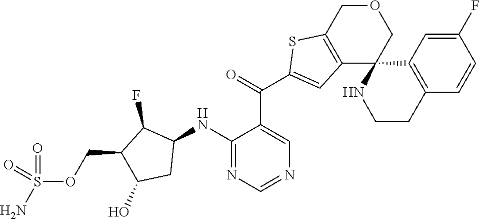 [SMILES: NS(=O)(=O)OC[C@H]1[C@@H](F)[C@@H](Nc2ncncc2C(=O)c2cc3c(s2)COC[C@@]32NCCc3ccc(F)cc32)C[C@@H]1O] | 608 |
| 18 | 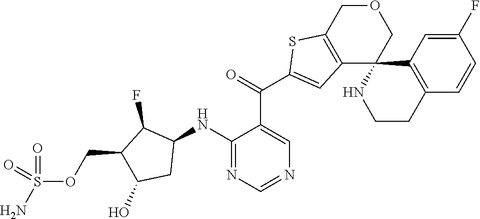 [SMILES: NS(=O)(=O)OC[C@H]1[C@@H](F)[C@@H](Nc2ncncc2C(=O)c2cc3c(s2)COC[C@]32NCCc3ccc(F)cc32)C[C@@H]1O] | 608 |
| 19 | 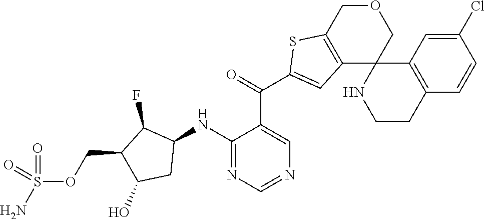 [SMILES: NS(=O)(=O)OC[C@H]1[C@@H](F)[C@@H](Nc2ncncc2C(=O)c2cc3c(s2)COCC32NCCc3ccc(Cl)cc32)C[C@@H]1O] | 624 |
| 20 | 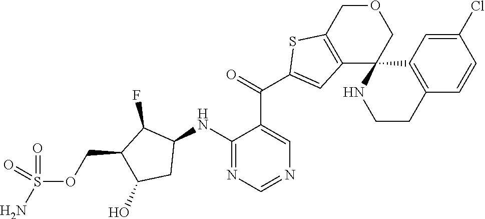 [SMILES: NS(=O)(=O)OC[C@H]1[C@@H](F)[C@@H](Nc2ncncc2C(=O)c2cc3c(s2)COC[C@@]32NCCc3ccc(Cl)cc32)C[C@@H]1O] | 624 |
| 21 | 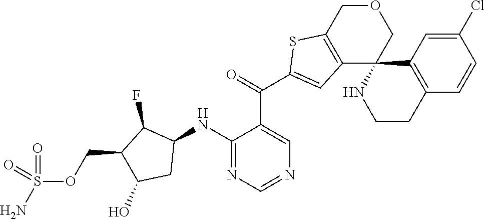 [SMILES: NS(=O)(=O)OC[C@H]1[C@@H](F)[C@@H](Nc2ncncc2C(=O)c2cc3c(s2)COC[C@]32NCCc3ccc(Cl)cc32)C[C@@H]1O] | 624 |
| 22 | 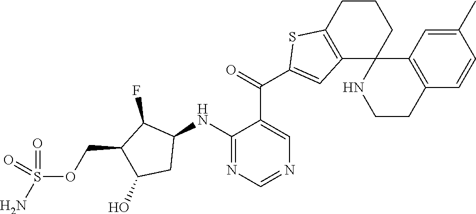 [SMILES: Cc1ccc2c(c1)C1(CCCc3sc(C(=O)c4cncnc4N[C@H]4C[C@H](O)[C@@H](COS(N)(=O)=O)[C@H]4F)cc31)NCC2] | 602 |
| 23 | 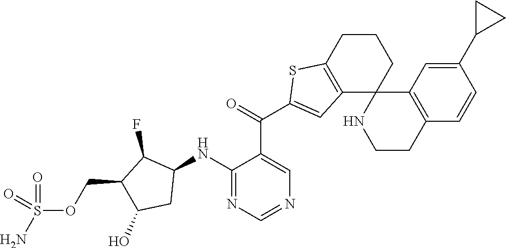 [SMILES: NS(=O)(=O)OC[C@H]1[C@@H](F)[C@@H](Nc2ncncc2C(=O)c2cc3c(s2)CCCC32NCCc3ccc(C4CC4)cc32)C[C@@H]1O] | 628 |
| 24 | 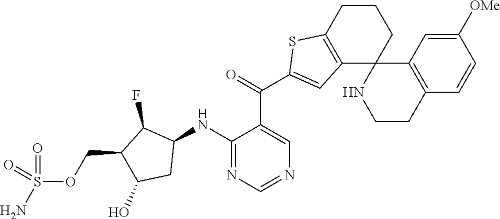 [SMILES: COc1ccc2c(c1)C1(CCCc3sc(C(=O)c4cncnc4N[C@H]4C[C@H](O)[C@@H](COS(N)(=O)=O)[C@H]4F)cc31)NCC2] | 618 |
| 25 | 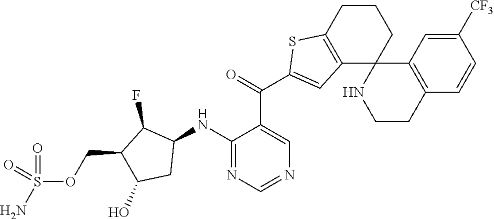 [SMILES: NS(=O)(=O)OC[C@H]1[C@@H](F)[C@@H](Nc2ncncc2C(=O)c2cc3c(s2)CCCC32NCCc3ccc(C(F)(F)F)cc32)C[C@@H]1O] | 656 |
| 26 | 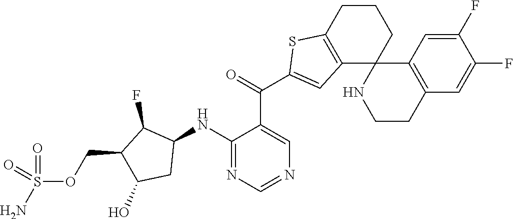 [SMILES: NS(=O)(=O)OC[C@H]1[C@@H](F)[C@@H](Nc2ncncc2C(=O)c2cc3c(s2)CCCC32NCCc3cc(F)c(F)cc32)C[C@@H]1O] | 624 |
| 27 | 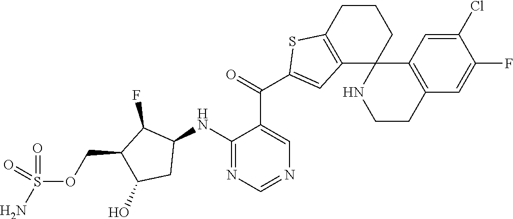 [SMILES: NS(=O)(=O)OC[C@H]1[C@@H](F)[C@@H](Nc2ncncc2C(=O)c2cc3c(s2)CCCC32NCCc3cc(F)c(Cl)cc32)C[C@@H]1O] | 640 |
| 28 | 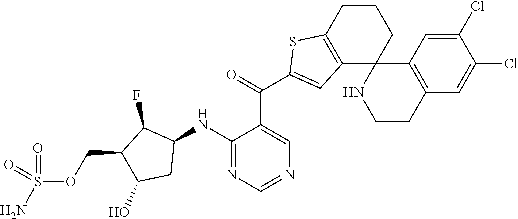 [SMILES: NS(=O)(=O)OC[C@H]1[C@@H](F)[C@@H](Nc2ncncc2C(=O)c2cc3c(s2)CCCC32NCCc3cc(Cl)c(Cl)cc32)C[C@@H]1O] | 656 |
| 29 | 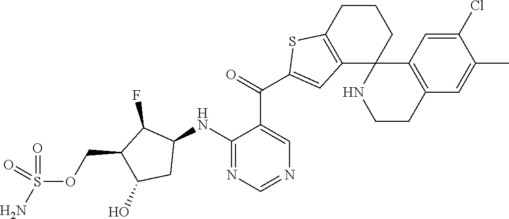 [SMILES: Cc1cc2c(cc1Cl)C1(CCCc3sc(C(=O)c4cncnc4N[C@H]4C[C@H](O)[C@@H](COS(N)(=O)=O)[C@H]4F)cc31)NCC2] | 636 |
| 30 | 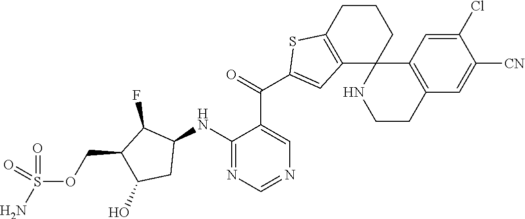 [SMILES: N#Cc1cc2c(cc1Cl)C1(CCCc3sc(C(=O)c4cncnc4N[C@H]4C[C@H](O)[C@@H](COS(N)(=O)=O)[C@H]4F)cc31)NCC2] | 647 |
| 31 | 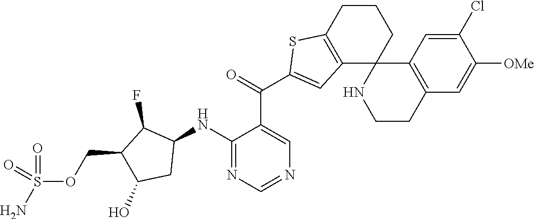 [SMILES: COc1cc2c(cc1Cl)C1(CCCc3sc(C(=O)c4cncnc4N[C@H]4C[C@H](O)[C@@H](COS(N)(=O)=O)[C@H]4F)cc31)NCC2] | 652 |
| 32 | 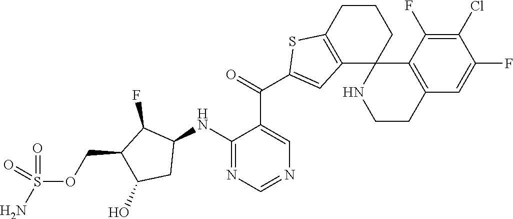 [SMILES: NS(=O)(=O)OC[C@H]1[C@@H](F)[C@@H](Nc2ncncc2C(=O)c2cc3c(s2)CCCC32NCCc3cc(F)c(Cl)c(F)c32)C[C@@H]1O] | 658 |
| 33 | 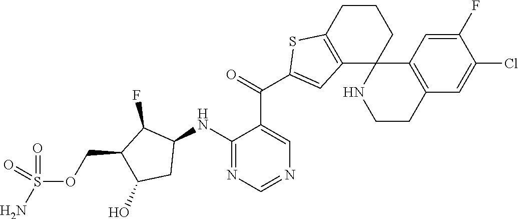 [SMILES: NS(=O)(=O)OC[C@H]1[C@@H](F)[C@@H](Nc2ncncc2C(=O)c2cc3c(s2)CCCC32NCCc3cc(Cl)c(F)cc32)C[C@@H]1O] | 640 |
| 34 | 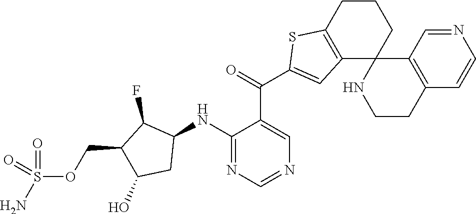 [SMILES: NS(=O)(=O)OC[C@H]1[C@@H](F)[C@@H](Nc2ncncc2C(=O)c2cc3c(s2)CCCC32NCCc3ccncc32)C[C@@H]1O] | 589 |
| 35 | 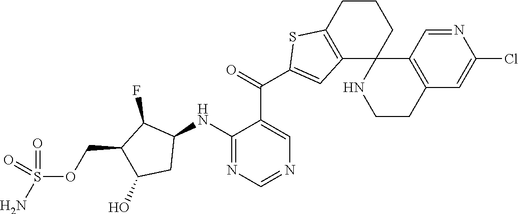 [SMILES: NS(=O)(=O)OC[C@H]1[C@@H](F)[C@@H](Nc2ncncc2C(=O)c2cc3c(s2)CCCC32NCCc3cc(Cl)ncc32)C[C@@H]1O] | 623 |
| 36 | 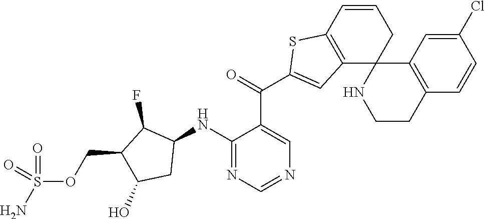 [SMILES: NS(=O)(=O)OC[C@H]1[C@@H](F)[C@@H](Nc2ncncc2C(=O)c2cc3c(s2)C=CCC32NCCc3ccc(Cl)cc32)C[C@@H]1O] | 620 |
| 37 | 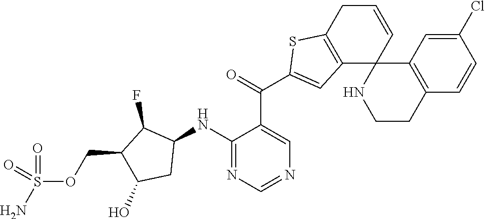 [SMILES: NS(=O)(=O)OC[C@H]1[C@@H](F)[C@@H](Nc2ncncc2C(=O)c2cc3c(s2)CC=CC32NCCc3ccc(Cl)cc32)C[C@@H]1O] | 620 |
| 38 | 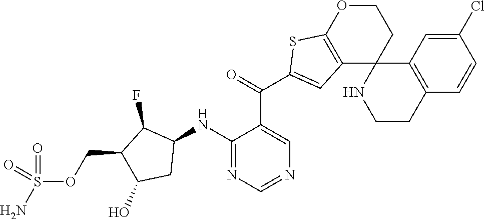 [SMILES: NS(=O)(=O)OC[C@H]1[C@@H](F)[C@@H](Nc2ncncc2C(=O)c2cc3c(s2)OCCC32NCCc3ccc(Cl)cc32)C[C@@H]1O] | 624 |
| 39 | 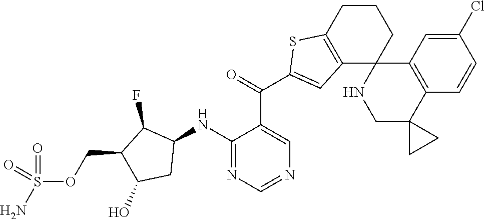 [SMILES: NS(=O)(=O)OC[C@H]1[C@@H](F)[C@@H](Nc2ncncc2C(=O)c2cc3c(s2)CCCC32NCC3(CC3)c3ccc(Cl)cc32)C[C@@H]1O] | 648 |
| 40 | 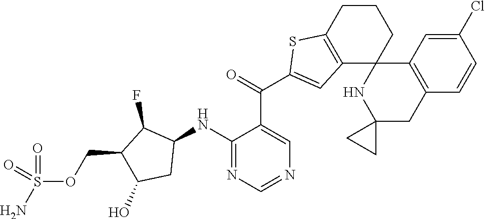 [SMILES: NS(=O)(=O)OC[C@H]1[C@@H](F)[C@@H](Nc2ncncc2C(=O)c2cc3c(s2)CCCC32NC3(CC3)Cc3ccc(Cl)cc32)C[C@@H]1O] | 648 |
| 41 | 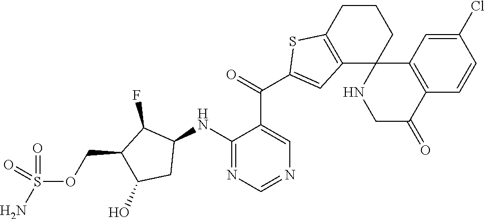 [SMILES: NS(=O)(=O)OC[C@H]1[C@@H](F)[C@@H](Nc2ncncc2C(=O)c2cc3c(s2)CCCC32NCC(=O)c3ccc(Cl)cc32)C[C@@H]1O] | 636 |
| 42 | 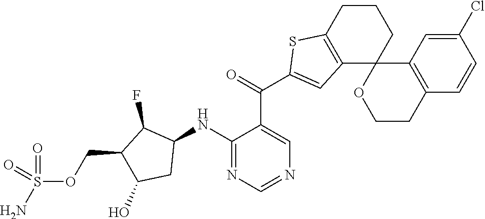 [SMILES: NS(=O)(=O)OC[C@H]1[C@@H](F)[C@@H](Nc2ncncc2C(=O)c2cc3c(s2)CCCC32OCCc3ccc(Cl)cc32)C[C@@H]1O] | 623 |
| 43 | 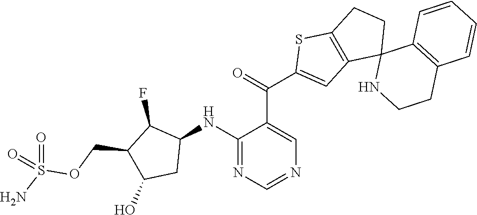 [SMILES: NS(=O)(=O)OC[C@H]1[C@@H](F)[C@@H](Nc2ncncc2C(=O)c2cc3c(s2)CCC32NCCc3ccccc32)C[C@@H]1O] | 574 |
| 44 | 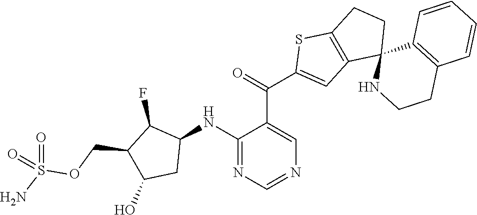 [SMILES: NS(=O)(=O)OC[C@H]1[C@@H](F)[C@@H](Nc2ncncc2C(=O)c2cc3c(s2)CC[C@@]32NCCc3ccccc32)C[C@@H]1O] | 574 |
| 45 | 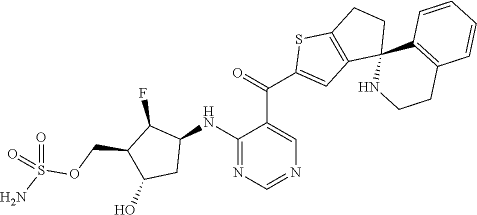 [SMILES: NS(=O)(=O)OC[C@H]1[C@@H](F)[C@@H](Nc2ncncc2C(=O)c2cc3c(s2)CC[C@]32NCCc3ccccc32)C[C@@H]1O] | 574 |
| 46 | 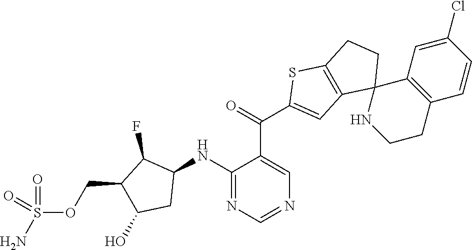 [SMILES: NS(=O)(=O)OC[C@H]1[C@@H](F)[C@@H](Nc2ncncc2C(=O)c2cc3c(s2)CCC32NCCc3ccc(Cl)cc32)C[C@@H]1O] | 608 |
| 47 | 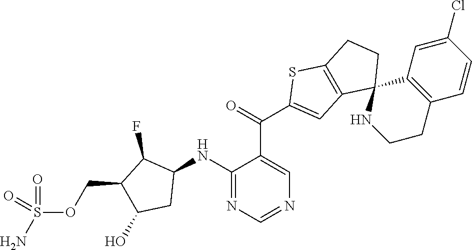 [SMILES: NS(=O)(=O)OC[C@H]1[C@@H](F)[C@@H](Nc2ncncc2C(=O)c2cc3c(s2)CC[C@@]32NCCc3ccc(Cl)cc32)C[C@@H]1O] | 608 |
| 48 | 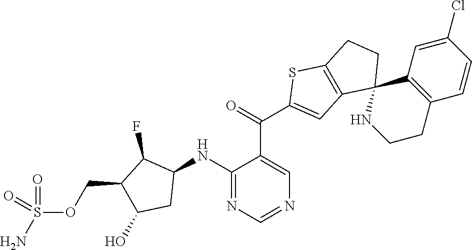 [SMILES: NS(=O)(=O)OC[C@H]1[C@@H](F)[C@@H](Nc2ncncc2C(=O)c2cc3c(s2)CC[C@]32NCCc3ccc(Cl)cc32)C[C@@H]1O] | 608 |
| 49 | 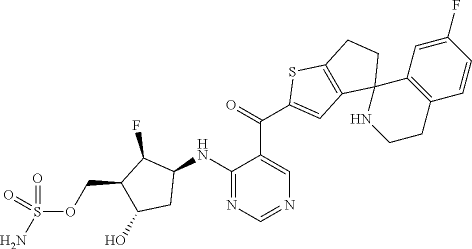 [SMILES: NS(=O)(=O)OC[C@H]1[C@@H](F)[C@@H](Nc2ncncc2C(=O)c2cc3c(s2)CCC32NCCc3ccc(F)cc32)C[C@@H]1O] | 592 |
| 50 | 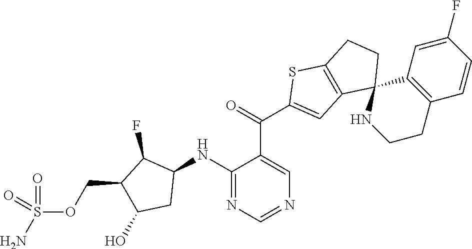 [SMILES: NS(=O)(=O)OC[C@H]1[C@@H](F)[C@@H](Nc2ncncc2C(=O)c2cc3c(s2)CC[C@@]32NCCc3ccc(F)cc32)C[C@@H]1O] | 592 |
| 51 | 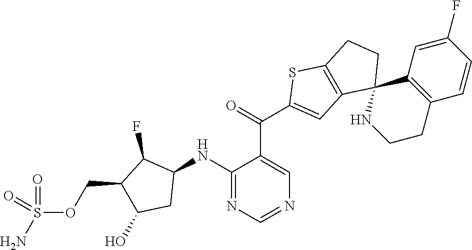 [SMILES: NS(=O)(=O)OC[C@H]1[C@@H](F)[C@@H](Nc2ncncc2C(=O)c2cc3c(s2)CC[C@]32NCCc3ccc(F)cc32)C[C@@H]1O] | 592 |
| 52 | 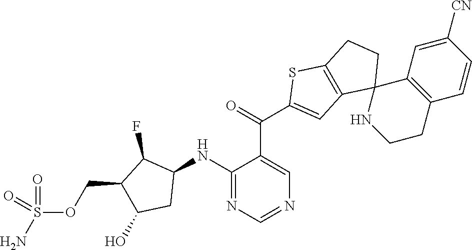 [SMILES: N#Cc1ccc2c(c1)C1(CCc3sc(C(=O)c4cncnc4N[C@H]4C[C@H](O)[C@@H](COS(N)(=O)=O)[C@H]4F)cc31)NCC2] | 599 |
| 53 | 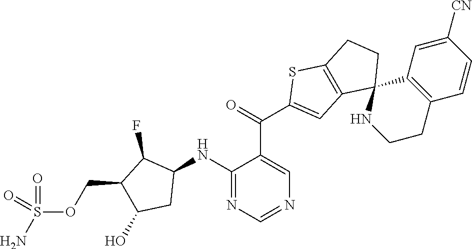 [SMILES: N#Cc1ccc2c(c1)[C@]1(CCc3sc(C(=O)c4cncnc4N[C@H]4C[C@H](O)[C@@H](COS(N)(=O)=O)[C@H]4F)cc31)NCC2] | 599 |
| 54 | 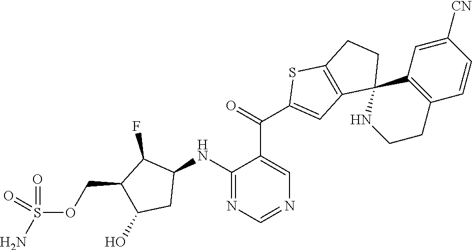 [SMILES: N#Cc1ccc2c(c1)[C@@]1(CCc3sc(C(=O)c4cncnc4N[C@H]4C[C@H](O)[C@@H](COS(N)(=O)=O)[C@H]4F)cc31)NCC2] | 599 |
| 55 | 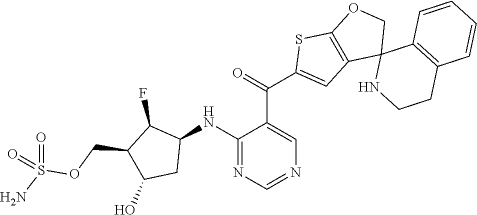 [SMILES: NS(=O)(=O)OC[C@H]1[C@@H](F)[C@@H](Nc2ncncc2C(=O)c2cc3c(s2)OCC32NCCc3ccccc32)C[C@@H]1O] | 576 |
| 56 | 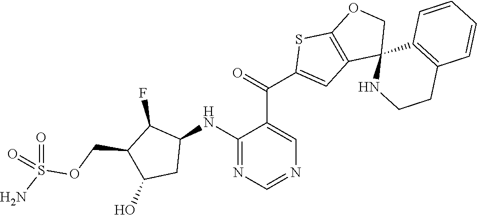 [SMILES: NS(=O)(=O)OC[C@H]1[C@@H](F)[C@@H](Nc2ncncc2C(=O)c2cc3c(s2)OC[C@@]32NCCc3ccccc32)C[C@@H]1O] | 576 |
| 57 | 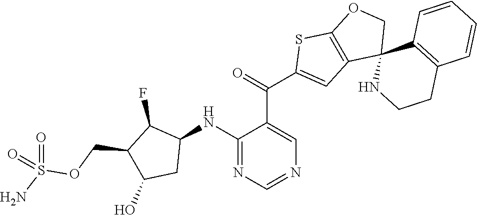 [SMILES: NS(=O)(=O)OC[C@H]1[C@@H](F)[C@@H](Nc2ncncc2C(=O)c2cc3c(s2)OC[C@]32NCCc3ccccc32)C[C@@H]1O] | 576 |
| 58 | 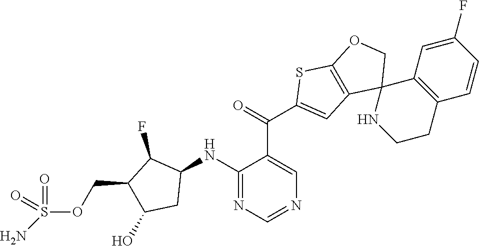 [SMILES: NS(=O)(=O)OC[C@H]1[C@@H](F)[C@@H](Nc2ncncc2C(=O)c2cc3c(s2)OCC32NCCc3ccc(F)cc32)C[C@@H]1O] | 594 |
| 59 | 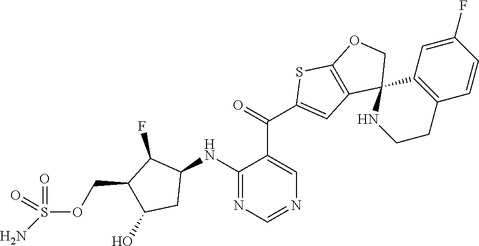 [SMILES: NS(=O)(=O)OC[C@H]1[C@@H](F)[C@@H](Nc2ncncc2C(=O)c2cc3c(s2)OC[C@@]32NCCc3ccc(F)cc32)C[C@@H]1O] | 594 |
| 60 | 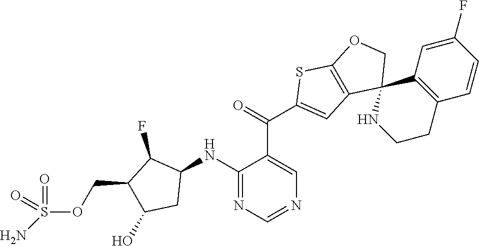 [SMILES: NS(=O)(=O)OC[C@H]1[C@@H](F)[C@@H](Nc2ncncc2C(=O)c2cc3c(s2)OC[C@]32NCCc3ccc(F)cc32)C[C@@H]1O] | 594 |
| 61 | 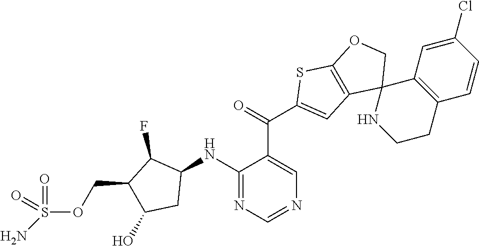 [SMILES: NS(=O)(=O)OC[C@H]1[C@@H](F)[C@@H](Nc2ncncc2C(=O)c2cc3c(s2)OCC32NCCc3ccc(Cl)cc32)C[C@@H]1O] | 610 |
| 62 | 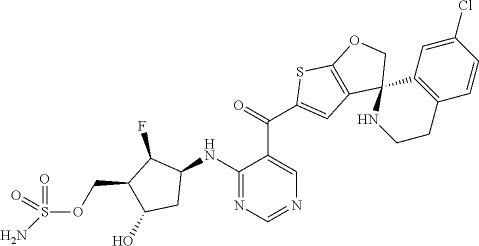 [SMILES: NS(=O)(=O)OC[C@H]1[C@@H](F)[C@@H](Nc2ncncc2C(=O)c2cc3c(s2)OC[C@@]32NCCc3ccc(Cl)cc32)C[C@@H]1O] | 610 |
| 63 | 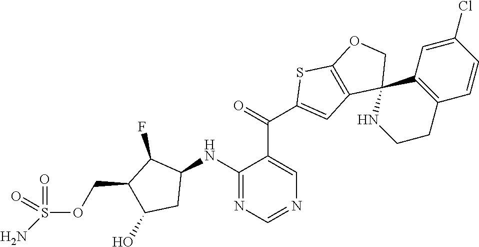 [SMILES: NS(=O)(=O)OC[C@H]1[C@@H](F)[C@@H](Nc2ncncc2C(=O)c2cc3c(s2)OC[C@]32NCCc3ccc(Cl)cc32)C[C@@H]1O] | 610 |
| 64 | 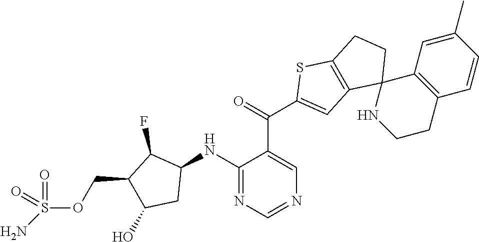 [SMILES: Cc1ccc2c(c1)C1(CCc3sc(C(=O)c4cncnc4N[C@H]4C[C@H](O)[C@@H](COS(N)(=O)=O)[C@H]4F)cc31)NCC2] | 588 |
| 65 | 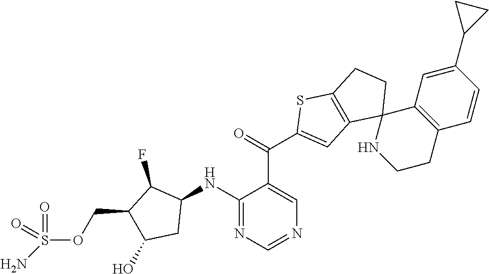 [SMILES: NS(=O)(=O)OC[C@H]1[C@@H](F)[C@@H](Nc2ncncc2C(=O)c2cc3c(s2)CCC32NCCc3ccc(C4CC4)cc32)C[C@@H]1O] | 614 |
| 66 | 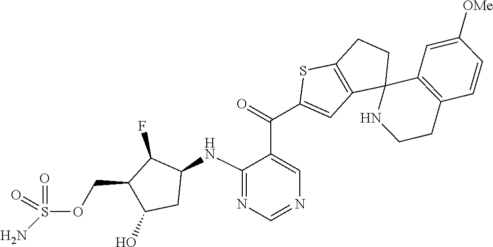 [SMILES: COc1ccc2c(c1)C1(CCc3sc(C(=O)c4cncnc4N[C@H]4C[C@H](O)[C@@H](COS(N)(=O)=O)[C@H]4F)cc31)NCC2] | 604 |
| 67 | 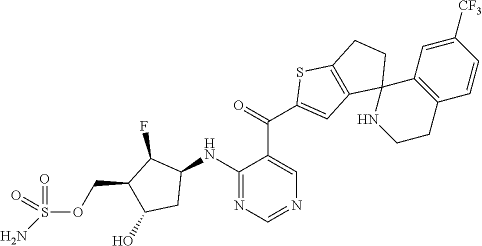 [SMILES: NS(=O)(=O)OC[C@H]1[C@@H](F)[C@@H](Nc2ncncc2C(=O)c2cc3c(s2)CCC32NCCc3ccc(C(F)(F)F)cc32)C[C@@H]1O] | 642 |
| 68 | 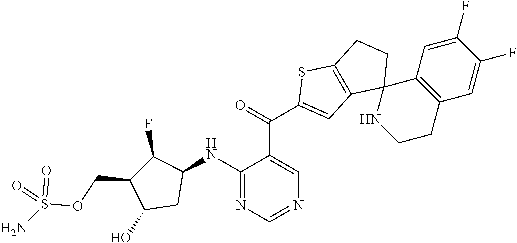 [SMILES: NS(=O)(=O)OC[C@H]1[C@@H](F)[C@@H](Nc2ncncc2C(=O)c2cc3c(s2)CCC32NCCc3cc(F)c(F)cc32)C[C@@H]1O] | 610 |
| 69 | 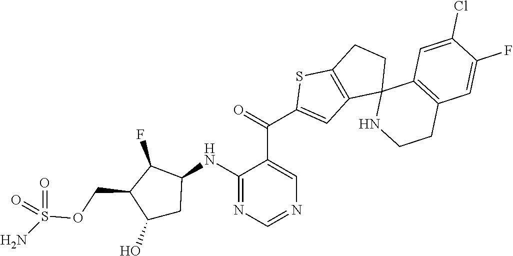 [SMILES: NS(=O)(=O)OC[C@H]1[C@@H](F)[C@@H](Nc2ncncc2C(=O)c2cc3c(s2)CCC32NCCc3cc(F)c(Cl)cc32)C[C@@H]1O] | 626 |
| 70 | 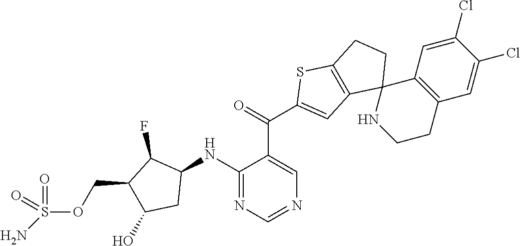 [SMILES: NS(=O)(=O)OC[C@H]1[C@@H](F)[C@@H](Nc2ncncc2C(=O)c2cc3c(s2)CCC32NCCc3cc(Cl)c(Cl)cc32)C[C@@H]1O] | 642 |
| 71 | 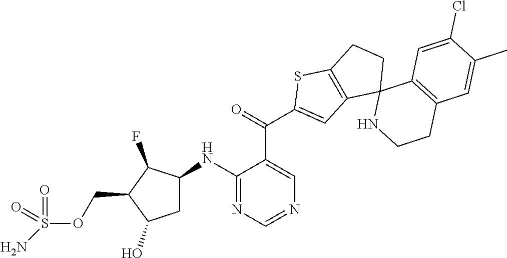 [SMILES: Cc1cc2c(cc1Cl)C1(CCc3sc(C(=O)c4cncnc4N[C@H]4C[C@H](O)[C@@H](COS(N)(=O)=O)[C@H]4F)cc31)NCC2] | 622 |
| 72 | 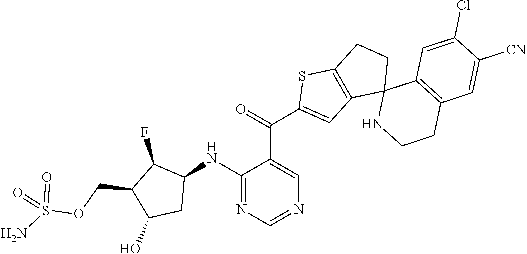 [SMILES: N#Cc1cc2c(cc1Cl)C1(CCc3sc(C(=O)c4cncnc4N[C@H]4C[C@H](O)[C@@H](COS(N)(=O)=O)[C@H]4F)cc31)NCC2] | 633 |
| 73 | 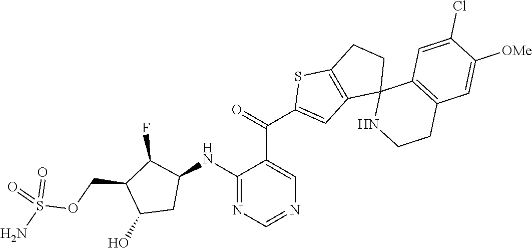 [SMILES: COc1cc2c(cc1Cl)C1(CCc3sc(C(=O)c4cncnc4N[C@H]4C[C@H](O)[C@@H](COS(N)(=O)=O)[C@H]4F)cc31)NCC2] | 638 |
| 74 | 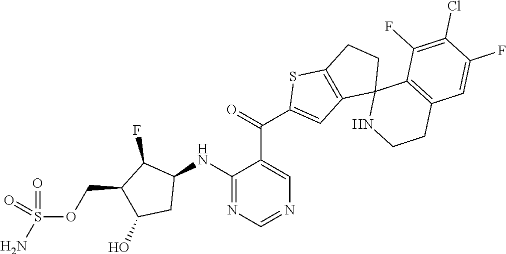 [SMILES: NS(=O)(=O)OC[C@H]1[C@@H](F)[C@@H](Nc2ncncc2C(=O)c2cc3c(s2)CCC32NCCc3cc(F)c(Cl)c(F)c32)C[C@@H]1O] | 644 |
| 75 | 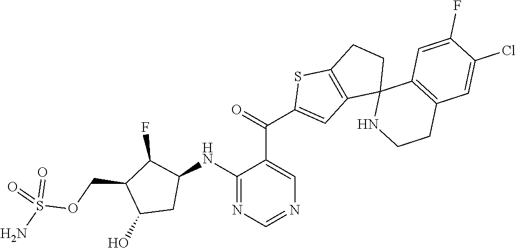 [SMILES: NS(=O)(=O)OC[C@H]1[C@@H](F)[C@@H](Nc2ncncc2C(=O)c2cc3c(s2)CCC32NCCc3cc(Cl)c(F)cc32)C[C@@H]1O] | 626 |
| 76 | 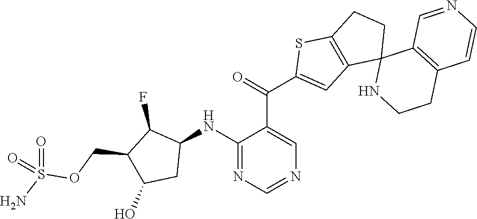 [SMILES: NS(=O)(=O)OC[C@H]1[C@@H](F)[C@@H](Nc2ncncc2C(=O)c2cc3c(s2)CCC32NCCc3ccncc32)C[C@@H]1O] | 575 |
| 77 | 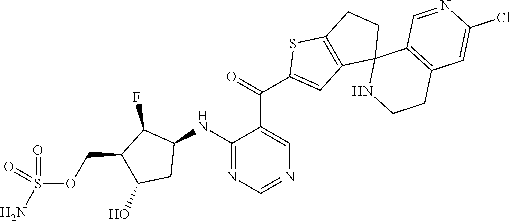 [SMILES: NS(=O)(=O)OC[C@H]1[C@@H](F)[C@@H](Nc2ncncc2C(=O)c2cc3c(s2)CCC32NCCc3cc(Cl)ncc32)C[C@@H]1O] | 609 |
| 78 | 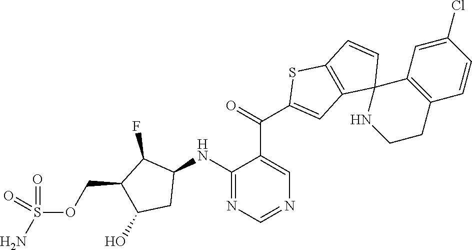 [SMILES: NS(=O)(=O)OC[C@H]1[C@@H](F)[C@@H](Nc2ncncc2C(=O)c2cc3c(s2)C=CC32NCCc3ccc(Cl)cc32)C[C@@H]1O] | 606 |
| 79 | 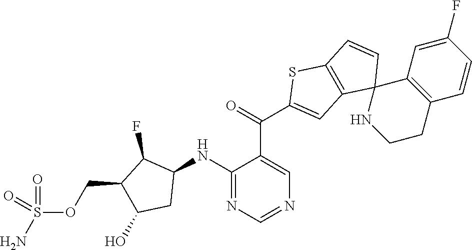 [SMILES: NS(=O)(=O)OC[C@H]1[C@@H](F)[C@@H](Nc2ncncc2C(=O)c2cc3c(s2)C=CC32NCCc3ccc(F)cc32)C[C@@H]1O] | 590 |
| 80 | 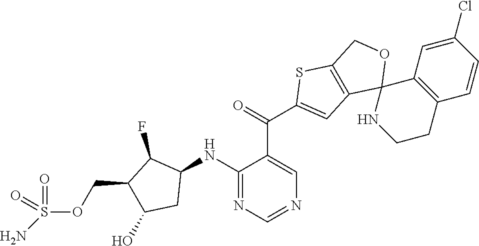 [SMILES: NS(=O)(=O)OC[C@H]1[C@@H](F)[C@@H](Nc2ncncc2C(=O)c2cc3c(s2)COC32NCCc3ccc(Cl)cc32)C[C@@H]1O] | 610 |
| 81 | 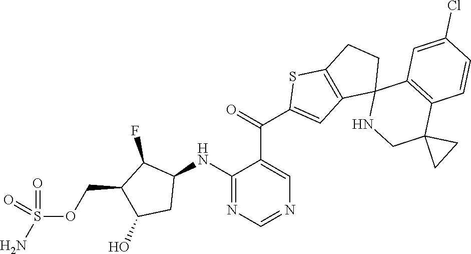 [SMILES: NS(=O)(=O)OC[C@H]1[C@@H](F)[C@@H](Nc2ncncc2C(=O)c2cc3c(s2)CCC32NCC3(CC3)c3ccc(Cl)cc32)C[C@@H]1O] | 634 |
| 82 | 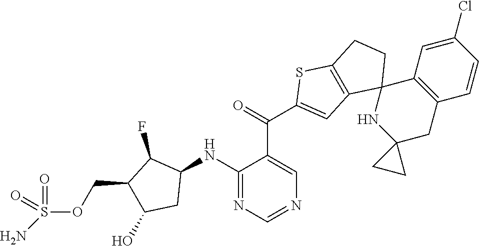 [SMILES: NS(=O)(=O)OC[C@H]1[C@@H](F)[C@@H](Nc2ncncc2C(=O)c2cc3c(s2)CCC32NC3(CC3)Cc3ccc(Cl)cc32)C[C@@H]1O] | 634 |
| 83 | 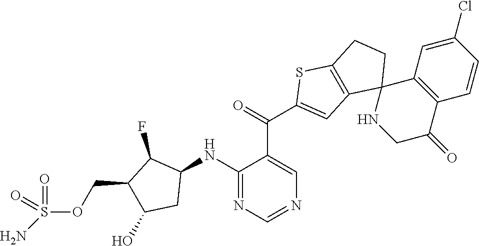 [SMILES: NS(=O)(=O)OC[C@H]1[C@@H](F)[C@@H](Nc2ncncc2C(=O)c2cc3c(s2)CCC32NCC(=O)c3ccc(Cl)cc32)C[C@@H]1O] | 622 |
| 84 | 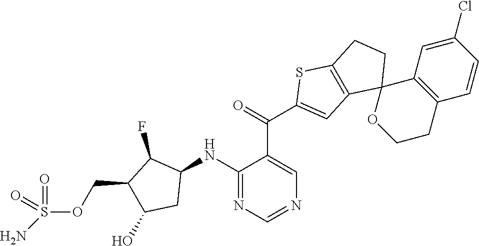 [SMILES: NS(=O)(=O)OC[C@H]1[C@@H](F)[C@@H](Nc2ncncc2C(=O)c2cc3c(s2)CCC32OCCc3ccc(Cl)cc32)C[C@@H]1O] | 609 |
| 85 | 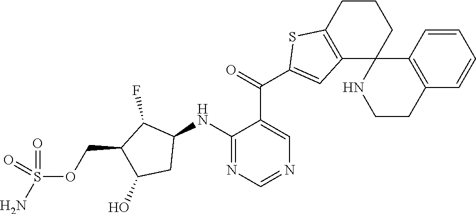 [SMILES: NS(=O)(=O)OC[C@H]1[C@H](F)[C@@H](Nc2ncncc2C(=O)c2cc3c(s2)CCCC32NCCc3ccccc32)C[C@@H]1O] | 588 |
| 86 | 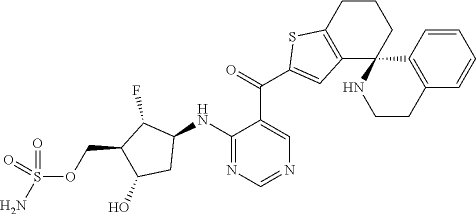 [SMILES: NS(=O)(=O)OC[C@H]1[C@H](F)[C@@H](Nc2ncncc2C(=O)c2cc3c(s2)CCC[C@@]32NCCc3ccccc32)C[C@@H]1O] | 588 |
| 87 | 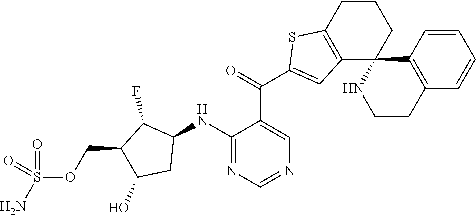 [SMILES: NS(=O)(=O)OC[C@H]1[C@H](F)[C@@H](Nc2ncncc2C(=O)c2cc3c(s2)CCC[C@]32NCCc3ccccc32)C[C@@H]1O] | 588 |
| 88 | 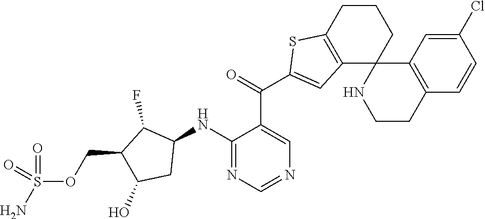 [SMILES: NS(=O)(=O)OC[C@H]1[C@H](F)[C@@H](Nc2ncncc2C(=O)c2cc3c(s2)CCCC32NCCc3ccc(Cl)cc32)C[C@@H]1O] | 622 |
| 89 | 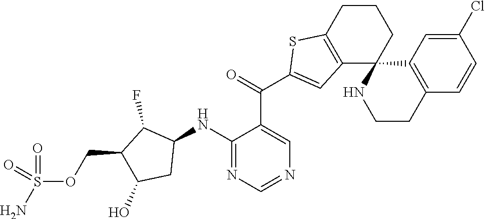 [SMILES: NS(=O)(=O)OC[C@H]1[C@H](F)[C@@H](Nc2ncncc2C(=O)c2cc3c(s2)CCC[C@@]32NCCc3ccc(Cl)cc32)C[C@@H]1O] | 622 |
| 90 | 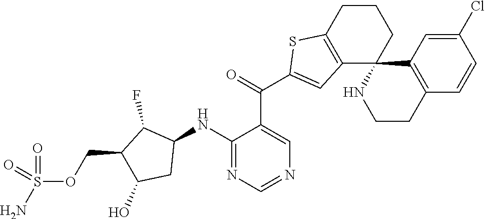 [SMILES: NS(=O)(=O)OC[C@H]1[C@H](F)[C@@H](Nc2ncncc2C(=O)c2cc3c(s2)CCC[C@]32NCCc3ccc(Cl)cc32)C[C@@H]1O] | 622 |
| 91 | 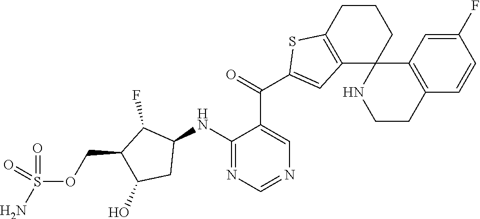 [SMILES: NS(=O)(=O)OC[C@H]1[C@H](F)[C@@H](Nc2ncncc2C(=O)c2cc3c(s2)CCCC32NCCc3ccc(F)cc32)C[C@@H]1O] | 606 |
| 92 | 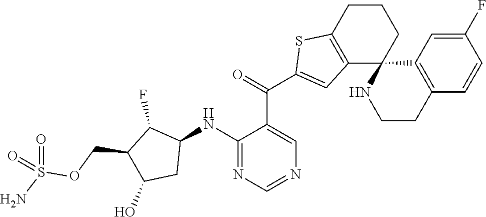 [SMILES: NS(=O)(=O)OC[C@H]1[C@H](F)[C@@H](Nc2ncncc2C(=O)c2cc3c(s2)CCC[C@@]32NCCc3ccc(F)cc32)C[C@@H]1O] | 606 |
| 93 | 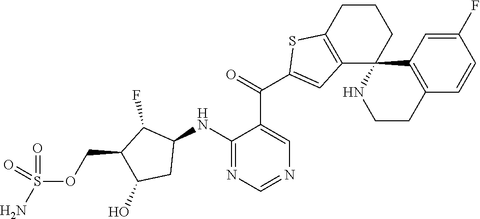 [SMILES: NS(=O)(=O)OC[C@H]1[C@H](F)[C@@H](Nc2ncncc2C(=O)c2cc3c(s2)CCC[C@]32NCCc3ccc(F)cc32)C[C@@H]1O] | 606 |
| 94 | 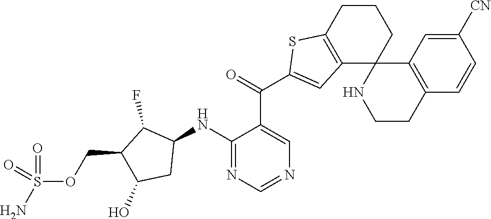 [SMILES: N#Cc1ccc2c(c1)C1(CCCc3sc(C(=O)c4cncnc4N[C@H]4C[C@H](O)[C@@H](COS(N)(=O)=O)[C@@H]4F)cc31)NCC2] | 613 |
| 95 | 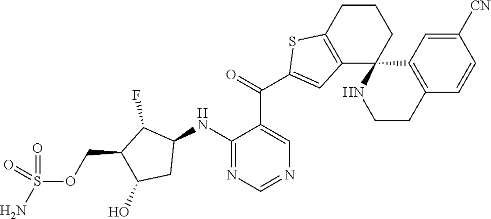 [SMILES: N#Cc1ccc2c(c1)[C@]1(CCCc3sc(C(=O)c4cncnc4N[C@H]4C[C@H](O)[C@@H](COS(N)(=O)=O)[C@@H]4F)cc31)NCC2] | 613 |
| 96 | 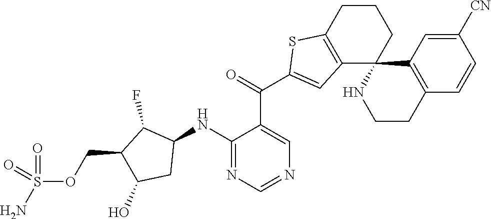 [SMILES: N#Cc1ccc2c(c1)[C@@]1(CCCc3sc(C(=O)c4cncnc4N[C@H]4C[C@H](O)[C@@H](COS(N)(=O)=O)[C@@H]4F)cc31)NCC2] | 613 |
| 97 | 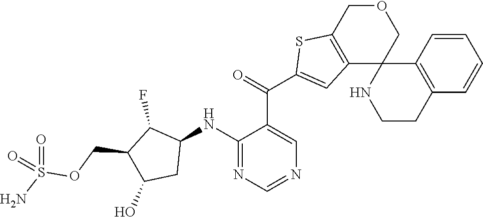 [SMILES: NS(=O)(=O)OC[C@H]1[C@H](F)[C@@H](Nc2ncncc2C(=O)c2cc3c(s2)COCC32NCCc3ccccc32)C[C@@H]1O] | 590 |
| 98 | 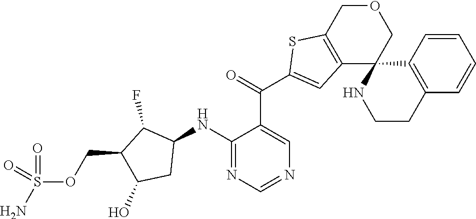 [SMILES: NS(=O)(=O)OC[C@H]1[C@H](F)[C@@H](Nc2ncncc2C(=O)c2cc3c(s2)COC[C@@]32NCCc3ccccc32)C[C@@H]1O] | 590 |
| 99 | 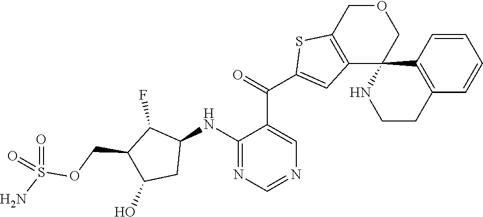 [SMILES: NS(=O)(=O)OC[C@H]1[C@H](F)[C@@H](Nc2ncncc2C(=O)c2cc3c(s2)COC[C@]32NCCc3ccccc32)C[C@@H]1O] | 590 |
| 100 | 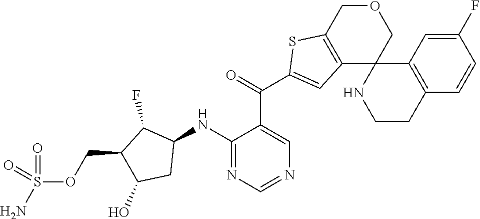 [SMILES: NS(=O)(=O)OC[C@H]1[C@H](F)[C@@H](Nc2ncncc2C(=O)c2cc3c(s2)COCC32NCCc3ccc(F)cc32)C[C@@H]1O] | 608 |
| 101 | 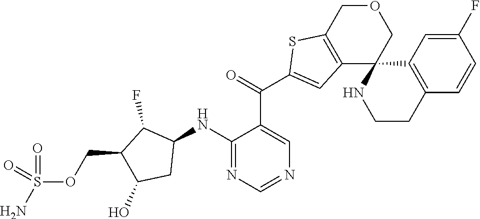 [SMILES: NS(=O)(=O)OC[C@H]1[C@H](F)[C@@H](Nc2ncncc2C(=O)c2cc3c(s2)COC[C@@]32NCCc3ccc(F)cc32)C[C@@H]1O] | 608 |
| 102 | 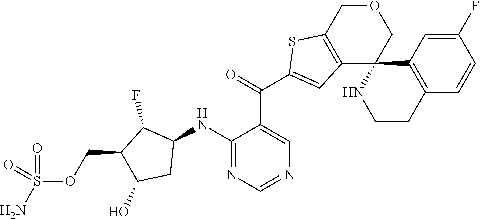 [SMILES: NS(=O)(=O)OC[C@H]1[C@H](F)[C@@H](Nc2ncncc2C(=O)c2cc3c(s2)COC[C@]32NCCc3ccc(F)cc32)C[C@@H]1O] | 608 |
| 103 | 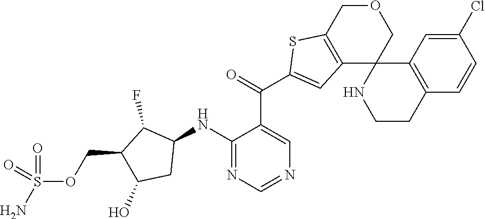 [SMILES: NS(=O)(=O)OC[C@H]1[C@H](F)[C@@H](Nc2ncncc2C(=O)c2cc3c(s2)COCC32NCCc3ccc(Cl)cc32)C[C@@H]1O] | 624 |
| 104 | 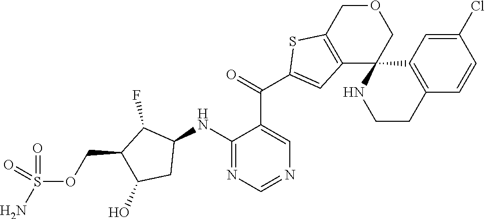 [SMILES: NS(=O)(=O)OC[C@H]1[C@H](F)[C@@H](Nc2ncncc2C(=O)c2cc3c(s2)COC[C@@]32NCCc3ccc(Cl)cc32)C[C@@H]1O] | 624 |
| 105 | 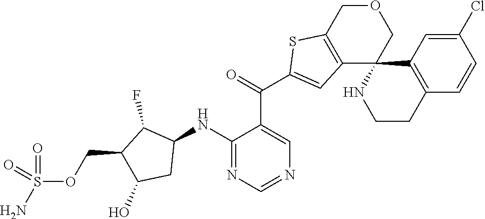 [SMILES: NS(=O)(=O)OC[C@H]1[C@H](F)[C@@H](Nc2ncncc2C(=O)c2cc3c(s2)COC[C@]32NCCc3ccc(Cl)cc32)C[C@@H]1O] | 624 |
| 106 | 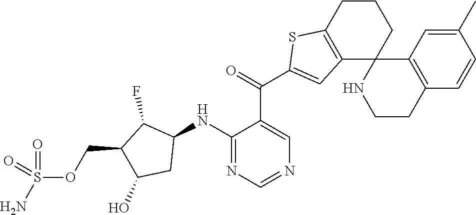 [SMILES: Cc1ccc2c(c1)C1(CCCc3sc(C(=O)c4cncnc4N[C@H]4C[C@H](O)[C@@H](COS(N)(=O)=O)[C@@H]4F)cc31)NCC2] | 602 |
| 107 | 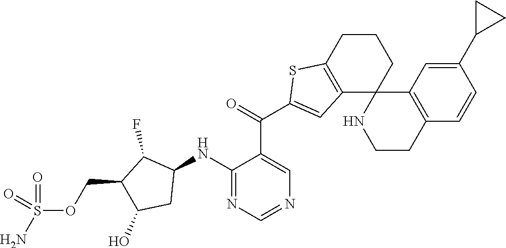 [SMILES: NS(=O)(=O)OC[C@H]1[C@H](F)[C@@H](Nc2ncncc2C(=O)c2cc3c(s2)CCCC32NCCc3ccc(C4CC4)cc32)C[C@@H]1O] | 628 |
| 108 | 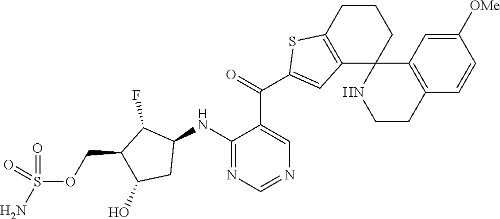 [SMILES: COc1ccc2c(c1)C1(CCCc3sc(C(=O)c4cncnc4N[C@H]4C[C@H](O)[C@@H](COS(N)(=O)=O)[C@@H]4F)cc31)NCC2] | 618 |
| 109 | 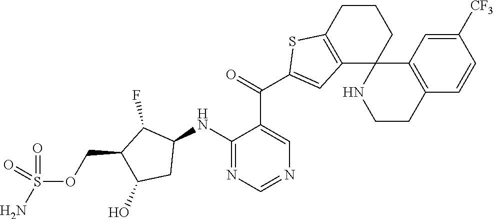 [SMILES: NS(=O)(=O)OC[C@H]1[C@H](F)[C@@H](Nc2ncncc2C(=O)c2cc3c(s2)CCCC32NCCc3ccc(C(F)(F)F)cc32)C[C@@H]1O] | 656 |
| 110 | 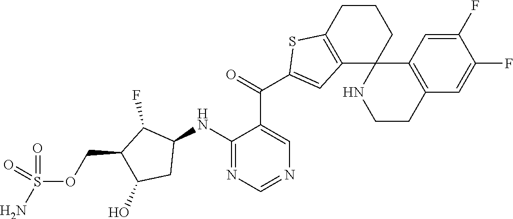 [SMILES: NS(=O)(=O)OC[C@H]1[C@H](F)[C@@H](Nc2ncncc2C(=O)c2cc3c(s2)CCCC32NCCc3cc(F)c(F)cc32)C[C@@H]1O] | 624 |
| 111 | 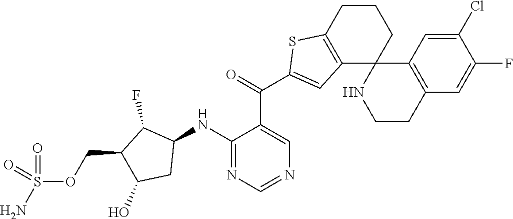 [SMILES: NS(=O)(=O)OC[C@H]1[C@H](F)[C@@H](Nc2ncncc2C(=O)c2cc3c(s2)CCCC32NCCc3cc(F)c(Cl)cc32)C[C@@H]1O] | 640 |
| 112 | 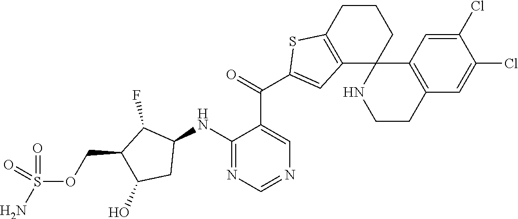 [SMILES: NS(=O)(=O)OC[C@H]1[C@H](F)[C@@H](Nc2ncncc2C(=O)c2cc3c(s2)CCCC32NCCc3cc(Cl)c(Cl)cc32)C[C@@H]1O] | 656 |
| 113 | 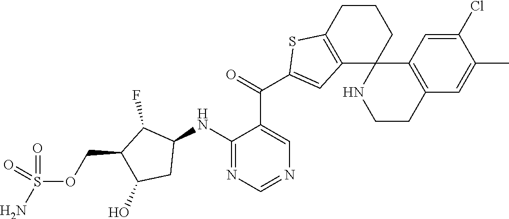 [SMILES: Cc1cc2c(cc1Cl)C1(CCCc3sc(C(=O)c4cncnc4N[C@H]4C[C@H](O)[C@@H](COS(N)(=O)=O)[C@@H]4F)cc31)NCC2] | 636 |
| 114 | 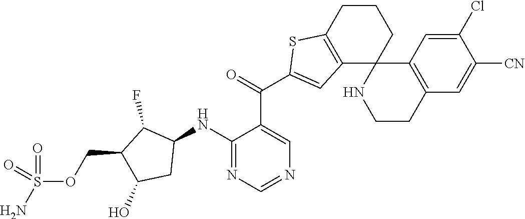 [SMILES: N#Cc1cc2c(cc1Cl)C1(CCCc3sc(C(=O)c4cncnc4N[C@H]4C[C@H](O)[C@@H](COS(N)(=O)=O)[C@@H]4F)cc31)NCC2] | 647 |
| 115 | 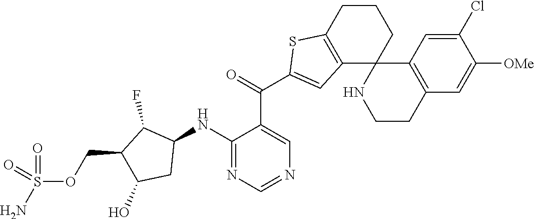 [SMILES: COc1cc2c(cc1Cl)C1(CCCc3sc(C(=O)c4cncnc4N[C@H]4C[C@H](O)[C@@H](COS(N)(=O)=O)[C@@H]4F)cc31)NCC2] | 652 |
| 116 | 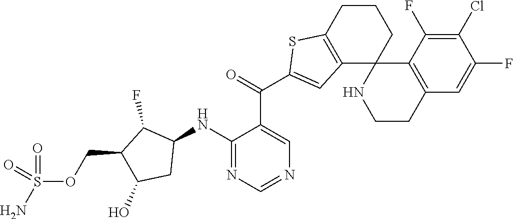 [SMILES: NS(=O)(=O)OC[C@H]1[C@H](F)[C@@H](Nc2ncncc2C(=O)c2cc3c(s2)CCCC32NCCc3cc(F)c(Cl)c(F)c32)C[C@@H]1O] | 658 |
| 117 | 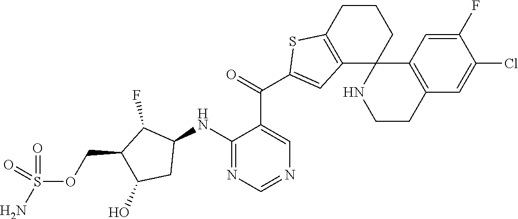 [SMILES: NS(=O)(=O)OC[C@H]1[C@H](F)[C@@H](Nc2ncncc2C(=O)c2cc3c(s2)CCCC32NCCc3cc(Cl)c(F)cc32)C[C@@H]1O] | 640 |
| 118 | 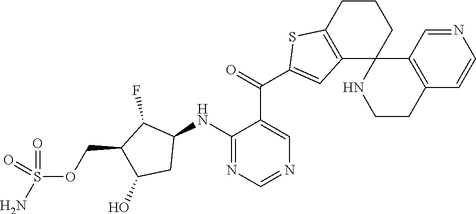 [SMILES: NS(=O)(=O)OC[C@H]1[C@H](F)[C@@H](Nc2ncncc2C(=O)c2cc3c(s2)CCCC32NCCc3ccncc32)C[C@@H]1O] | 589 |
| 119 | 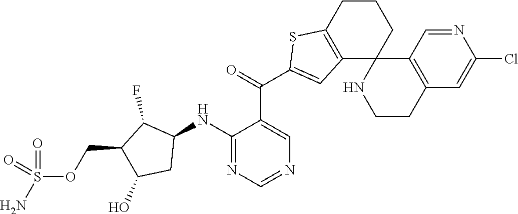 [SMILES: NS(=O)(=O)OC[C@H]1[C@H](F)[C@@H](Nc2ncncc2C(=O)c2cc3c(s2)CCCC32NCCc3cc(Cl)ncc32)C[C@@H]1O] | 623 |
| 120 | 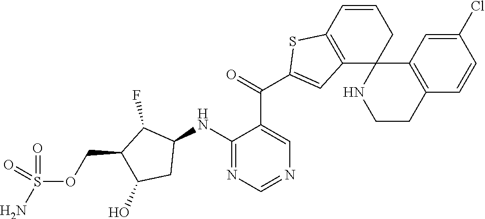 [SMILES: NS(=O)(=O)OC[C@H]1[C@H](F)[C@@H](Nc2ncncc2C(=O)c2cc3c(s2)C=CCC32NCCc3ccc(Cl)cc32)C[C@@H]1O] | 620 |
| 121 | 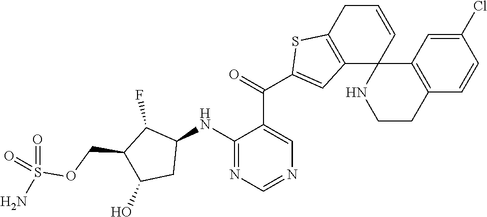 [SMILES: NS(=O)(=O)OC[C@H]1[C@H](F)[C@@H](Nc2ncncc2C(=O)c2cc3c(s2)CC=CC32NCCc3ccc(Cl)cc32)C[C@@H]1O] | 620 |
| 122 | 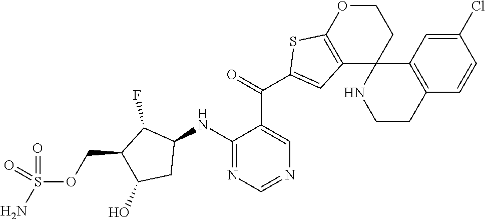 [SMILES: NS(=O)(=O)OC[C@H]1[C@H](F)[C@@H](Nc2ncncc2C(=O)c2cc3c(s2)OCCC32NCCc3ccc(Cl)cc32)C[C@@H]1O] | 624 |
| 123 | 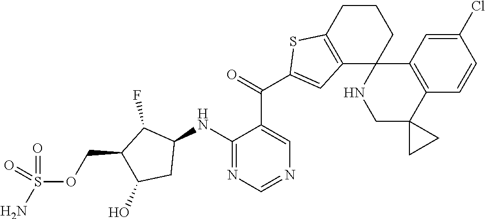 [SMILES: NS(=O)(=O)OC[C@H]1[C@H](F)[C@@H](Nc2ncncc2C(=O)c2cc3c(s2)CCCC32NCC3(CC3)c3ccc(Cl)cc32)C[C@@H]1O] | 648 |
| 124 | 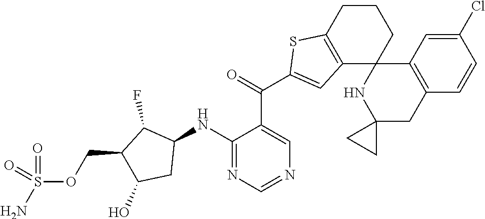 [SMILES: NS(=O)(=O)OC[C@H]1[C@H](F)[C@@H](Nc2ncncc2C(=O)c2cc3c(s2)CCCC32NC3(CC3)Cc3ccc(Cl)cc32)C[C@@H]1O] | 648 |
| 125 | 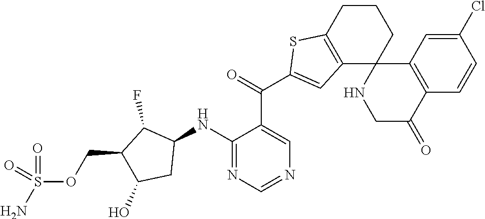 [SMILES: NS(=O)(=O)OC[C@H]1[C@H](F)[C@@H](Nc2ncncc2C(=O)c2cc3c(s2)CCCC32NCC(=O)c3ccc(Cl)cc32)C[C@@H]1O] | 636 |
| 126 | 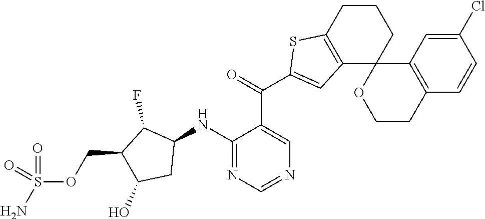 [SMILES: NS(=O)(=O)OC[C@H]1[C@H](F)[C@@H](Nc2ncncc2C(=O)c2cc3c(s2)CCCC32OCCc3ccc(Cl)cc32)C[C@@H]1O] | 623 |
| 127 | 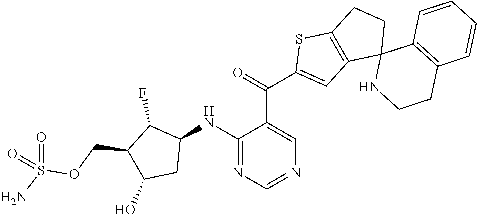 [SMILES: NS(=O)(=O)OC[C@H]1[C@H](F)[C@@H](Nc2ncncc2C(=O)c2cc3c(s2)CCC32NCCc3ccccc32)C[C@@H]1O] | 574 |
| 128 | 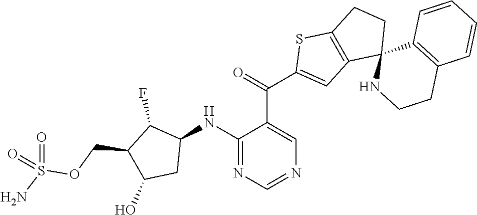 [SMILES: NS(=O)(=O)OC[C@H]1[C@H](F)[C@@H](Nc2ncncc2C(=O)c2cc3c(s2)CC[C@@]32NCCc3ccccc32)C[C@@H]1O] | 574 |
| 129 | 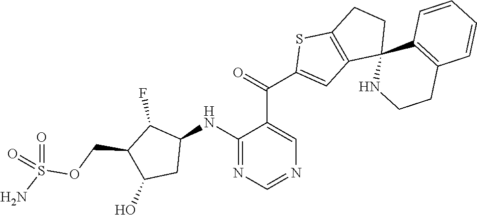 [SMILES: NS(=O)(=O)OC[C@H]1[C@H](F)[C@@H](Nc2ncncc2C(=O)c2cc3c(s2)CC[C@]32NCCc3ccccc32)C[C@@H]1O] | 574 |
| 130 | 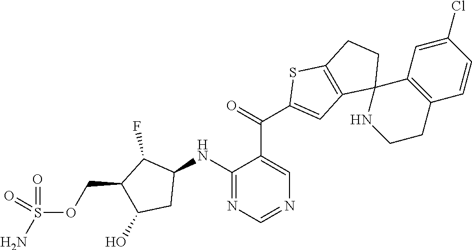 [SMILES: NS(=O)(=O)OC[C@H]1[C@H](F)[C@@H](Nc2ncncc2C(=O)c2cc3c(s2)CCC32NCCc3ccc(Cl)cc32)C[C@@H]1O] | 608 |
| 131 | 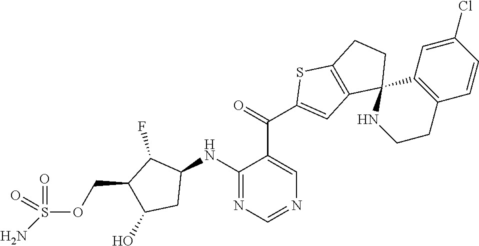 [SMILES: NS(=O)(=O)OC[C@H]1[C@H](F)[C@@H](Nc2ncncc2C(=O)c2cc3c(s2)CC[C@@]32NCCc3ccc(Cl)cc32)C[C@@H]1O] | 608 |
| 132 | 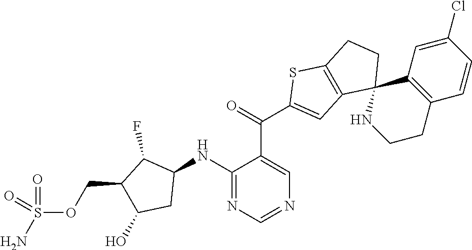 [SMILES: NS(=O)(=O)OC[C@H]1[C@H](F)[C@@H](Nc2ncncc2C(=O)c2cc3c(s2)CC[C@]32NCCc3ccc(Cl)cc32)C[C@@H]1O] | 608 |
| 133 | 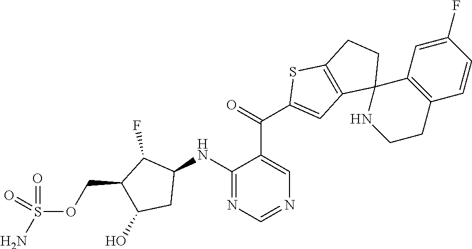 [SMILES: NS(=O)(=O)OC[C@H]1[C@H](F)[C@@H](Nc2ncncc2C(=O)c2cc3c(s2)CCC32NCCc3ccc(F)cc32)C[C@@H]1O] | 592 |
| 134 | 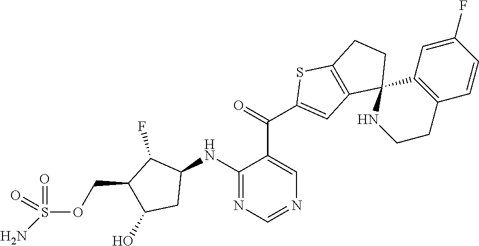 [SMILES: NS(=O)(=O)OC[C@H]1[C@H](F)[C@@H](Nc2ncncc2C(=O)c2cc3c(s2)CC[C@@]32NCCc3ccc(F)cc32)C[C@@H]1O] | 592 |
| 135 | 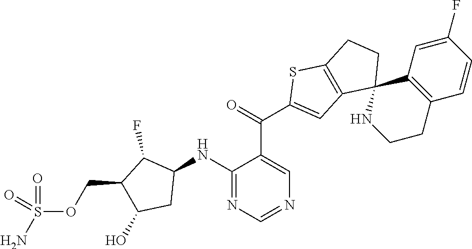 [SMILES: NS(=O)(=O)OC[C@H]1[C@H](F)[C@@H](Nc2ncncc2C(=O)c2cc3c(s2)CC[C@]32NCCc3ccc(F)cc32)C[C@@H]1O] | 592 |
| 136 | 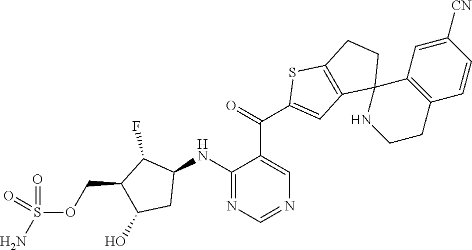 [SMILES: N#Cc1ccc2c(c1)C1(CCc3sc(C(=O)c4cncnc4N[C@H]4C[C@H](O)[C@@H](COS(N)(=O)=O)[C@@H]4F)cc31)NCC2] | 599 |
| 137 | 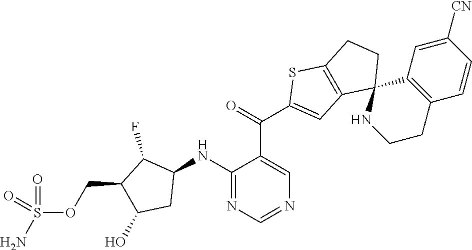 [SMILES: N#Cc1ccc2c(c1)[C@]1(CCc3sc(C(=O)c4cncnc4N[C@H]4C[C@H](O)[C@@H](COS(N)(=O)=O)[C@@H]4F)cc31)NCC2] | 599 |
| 138 | 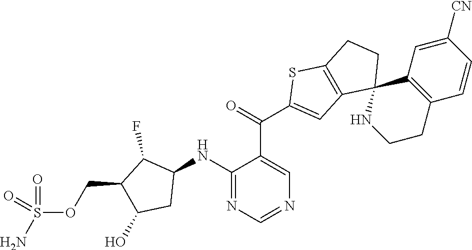 [SMILES: N#Cc1ccc2c(c1)[C@@]1(CCc3sc(C(=O)c4cncnc4N[C@H]4C[C@H](O)[C@@H](COS(N)(=O)=O)[C@@H]4F)cc31)NCC2] | 599 |
| 139 | 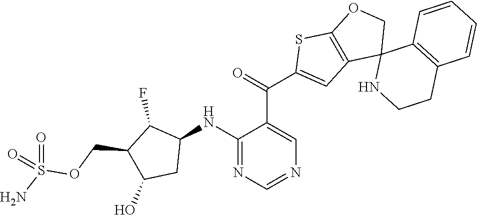 [SMILES: NS(=O)(=O)OC[C@H]1[C@H](F)[C@@H](Nc2ncncc2C(=O)c2cc3c(s2)OCC32NCCc3ccccc32)C[C@@H]1O] | 576 |
| 140 | 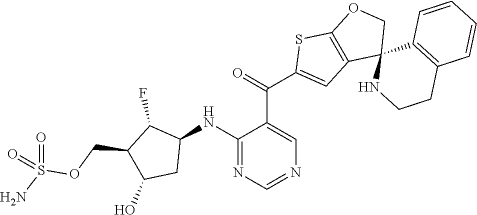 [SMILES: NS(=O)(=O)OC[C@H]1[C@H](F)[C@@H](Nc2ncncc2C(=O)c2cc3c(s2)OC[C@@]32NCCc3ccccc32)C[C@@H]1O] | 576 |
| 141 | 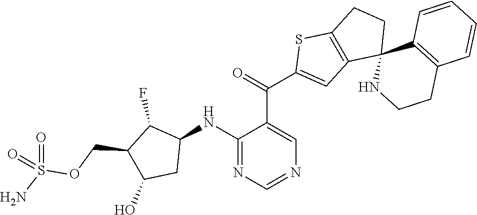 [SMILES: NS(=O)(=O)OC[C@H]1[C@H](F)[C@@H](Nc2ncncc2C(=O)c2cc3c(s2)CC[C@]32NCCc3ccccc32)C[C@@H]1O] | 576 |
| 142 | 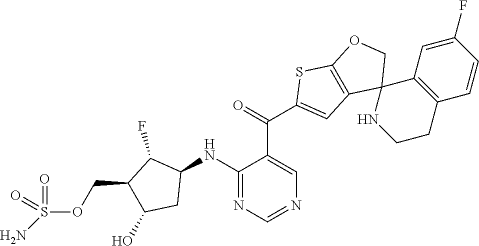 [SMILES: NS(=O)(=O)OC[C@H]1[C@H](F)[C@@H](Nc2ncncc2C(=O)c2cc3c(s2)OCC32NCCc3ccc(F)cc32)C[C@@H]1O] | 594 |
| 143 | 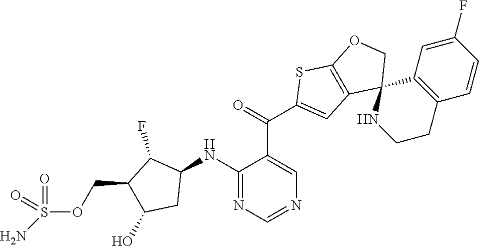 [SMILES: NS(=O)(=O)OC[C@H]1[C@H](F)[C@@H](Nc2ncncc2C(=O)c2cc3c(s2)OC[C@@]32NCCc3ccc(F)cc32)C[C@@H]1O] | 594 |
| 144 | 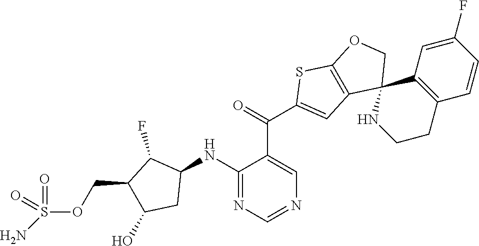 [SMILES: NS(=O)(=O)OC[C@H]1[C@H](F)[C@@H](Nc2ncncc2C(=O)c2cc3c(s2)OC[C@]32NCCc3ccc(F)cc32)C[C@@H]1O] | 594 |
| 145 | 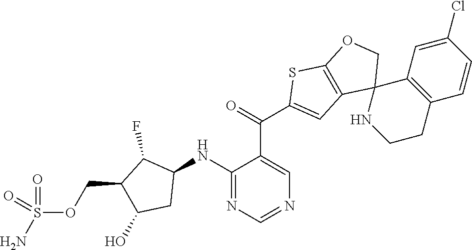 [SMILES: NS(=O)(=O)OC[C@H]1[C@H](F)[C@@H](Nc2ncncc2C(=O)c2cc3c(s2)OCC32NCCc3ccc(Cl)cc32)C[C@@H]1O] | 610 |
| 146 | 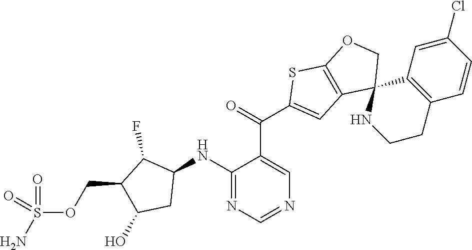 [SMILES: NS(=O)(=O)OC[C@H]1[C@H](F)[C@@H](Nc2ncncc2C(=O)c2cc3c(s2)OC[C@@]32NCCc3ccc(Cl)cc32)C[C@@H]1O] | 610 |
| 147 | 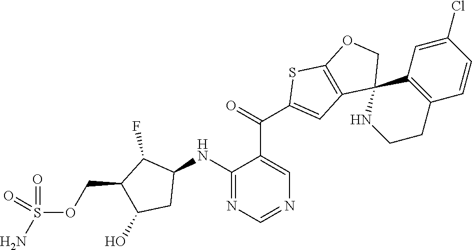 [SMILES: NS(=O)(=O)OC[C@H]1[C@H](F)[C@@H](Nc2ncncc2C(=O)c2cc3c(s2)OC[C@]32NCCc3ccc(Cl)cc32)C[C@@H]1O] | 610 |
| 148 | 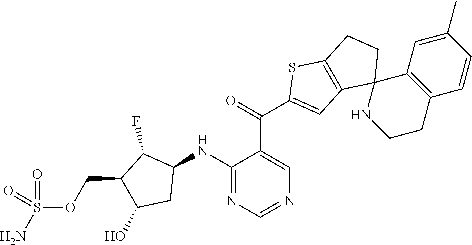 [SMILES: Cc1ccc2c(c1)C1(CCc3sc(C(=O)c4cncnc4N[C@H]4C[C@H](O)[C@@H](COS(N)(=O)=O)[C@@H]4F)cc31)NCC2] | 588 |
| 149 | 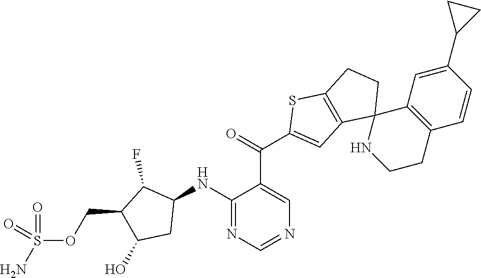 [SMILES: NS(=O)(=O)OC[C@H]1[C@H](F)[C@@H](Nc2ncncc2C(=O)c2cc3c(s2)CCC32NCCc3ccc(C4CC4)cc32)C[C@@H]1O] | 614 |
| 150 | 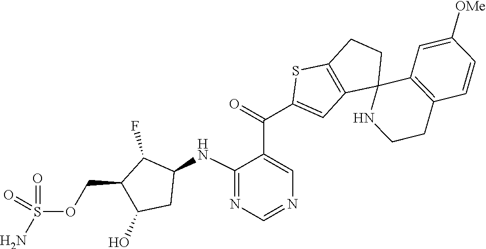 [SMILES: COc1ccc2c(c1)C1(CCc3sc(C(=O)c4cncnc4N[C@H]4C[C@H](O)[C@@H](COS(N)(=O)=O)[C@@H]4F)cc31)NCC2] | 604 |
| 151 | 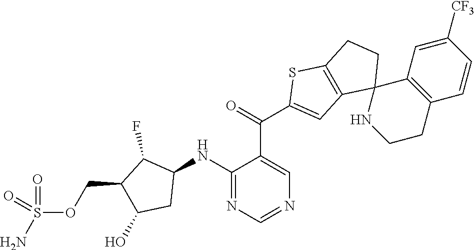 [SMILES: NS(=O)(=O)OC[C@H]1[C@H](F)[C@@H](Nc2ncncc2C(=O)c2cc3c(s2)CCC32NCCc3ccc(C(F)(F)F)cc32)C[C@@H]1O] | 642 |
| 152 | 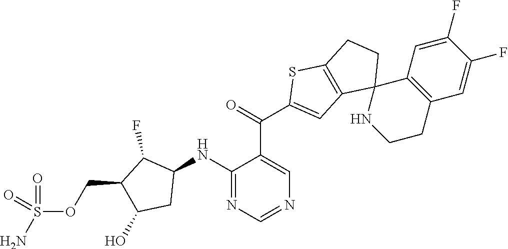 [SMILES: NS(=O)(=O)OC[C@H]1[C@H](F)[C@@H](Nc2ncncc2C(=O)c2cc3c(s2)CCC32NCCc3cc(F)c(F)cc32)C[C@@H]1O] | 610 |
| 153 | 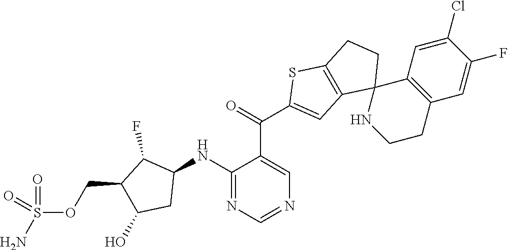 [SMILES: NS(=O)(=O)OC[C@H]1[C@H](F)[C@@H](Nc2ncncc2C(=O)c2cc3c(s2)CCC32NCCc3cc(F)c(Cl)cc32)C[C@@H]1O] | 626 |
| 154 | 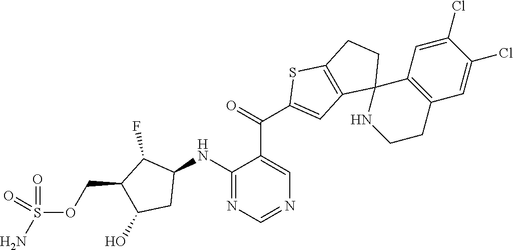 [SMILES: NS(=O)(=O)OC[C@H]1[C@H](F)[C@@H](Nc2ncncc2C(=O)c2cc3c(s2)CCC32NCCc3cc(Cl)c(Cl)cc32)C[C@@H]1O] | 642 |
| 155 | 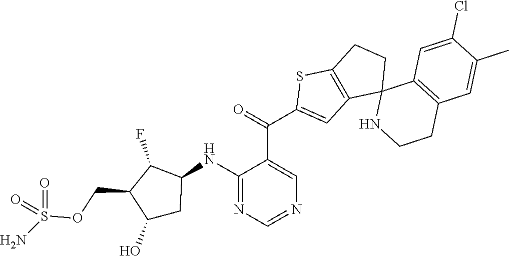 [SMILES: Cc1cc2c(cc1Cl)C1(CCc3sc(C(=O)c4cncnc4N[C@H]4C[C@H](O)[C@@H](COS(N)(=O)=O)[C@@H]4F)cc31)NCC2] | 622 |
| 156 | 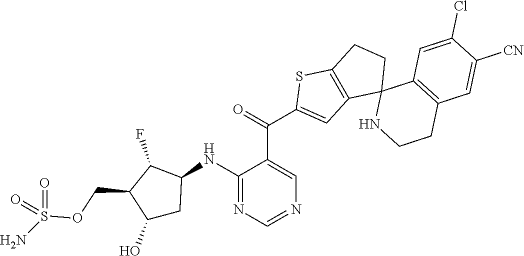 [SMILES: N#Cc1cc2c(cc1Cl)C1(CCc3sc(C(=O)c4cncnc4N[C@H]4C[C@H](O)[C@@H](COS(N)(=O)=O)[C@@H]4F)cc31)NCC2] | 633 |
| 157 | 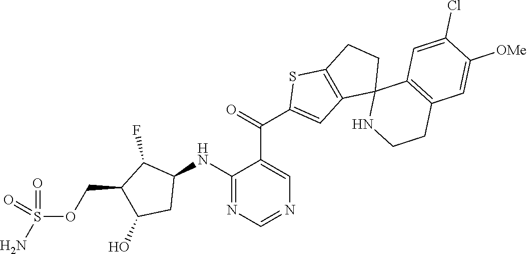 [SMILES: COc1cc2c(cc1Cl)C1(CCc3sc(C(=O)c4cncnc4N[C@H]4C[C@H](O)[C@@H](COS(N)(=O)=O)[C@@H]4F)cc31)NCC2] | 638 |
| 158 | 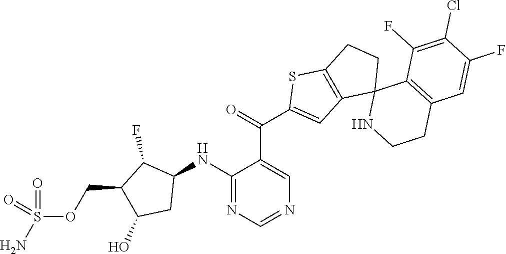 [SMILES: NS(=O)(=O)OC[C@H]1[C@H](F)[C@@H](Nc2ncncc2C(=O)c2cc3c(s2)CCC32NCCc3cc(F)c(Cl)c(F)c32)C[C@@H]1O] | 644 |
| 159 | 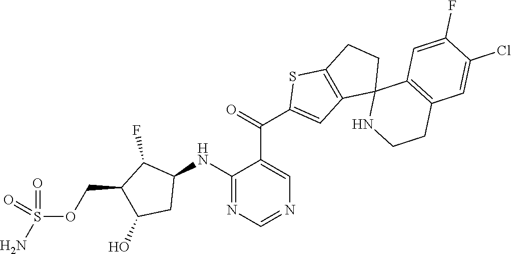 [SMILES: NS(=O)(=O)OC[C@H]1[C@H](F)[C@@H](Nc2ncncc2C(=O)c2cc3c(s2)CCC32NCCc3cc(Cl)c(F)cc32)C[C@@H]1O] | 626 |
| 160 | 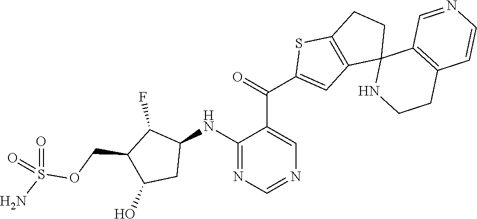 [SMILES: NS(=O)(=O)OC[C@H]1[C@H](F)[C@@H](Nc2ncncc2C(=O)c2cc3c(s2)CCC32NCCc3ccncc32)C[C@@H]1O] | 575 |
| 161 | 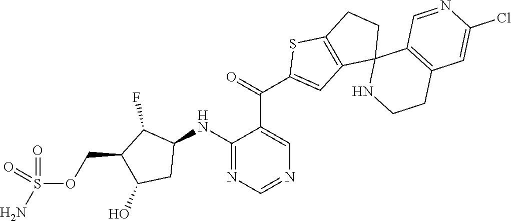 [SMILES: NS(=O)(=O)OC[C@H]1[C@H](F)[C@@H](Nc2ncncc2C(=O)c2cc3c(s2)CCC32NCCc3cc(Cl)ncc32)C[C@@H]1O] | 609 |
| 162 | 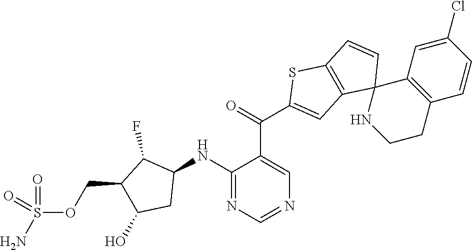 [SMILES: NS(=O)(=O)OC[C@H]1[C@H](F)[C@@H](Nc2ncncc2C(=O)c2cc3c(s2)C=CC32NCCc3ccc(Cl)cc32)C[C@@H]1O] | 606 |
| 163 | 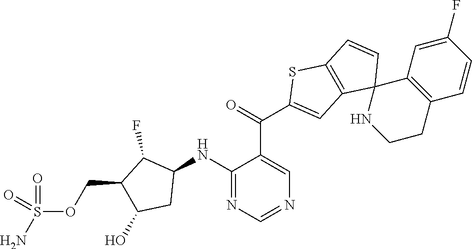 [SMILES: NS(=O)(=O)OC[C@H]1[C@H](F)[C@@H](Nc2ncncc2C(=O)c2cc3c(s2)C=CC32NCCc3ccc(F)cc32)C[C@@H]1O] | 590 |
| 164 | 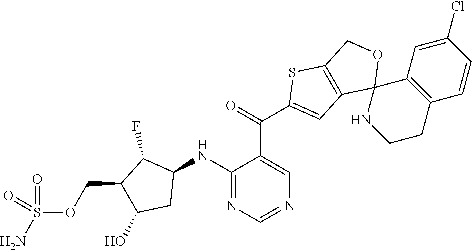 [SMILES: NS(=O)(=O)OC[C@H]1[C@H](F)[C@@H](Nc2ncncc2C(=O)c2cc3c(s2)COC32NCCc3ccc(Cl)cc32)C[C@@H]1O] | 610 |
| 165 | 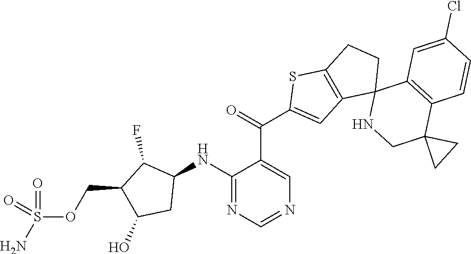 [SMILES: NS(=O)(=O)OC[C@H]1[C@H](F)[C@@H](Nc2ncncc2C(=O)c2cc3c(s2)CCC32NCC3(CC3)c3ccc(Cl)cc32)C[C@@H]1O] | 634 |
| 166 | 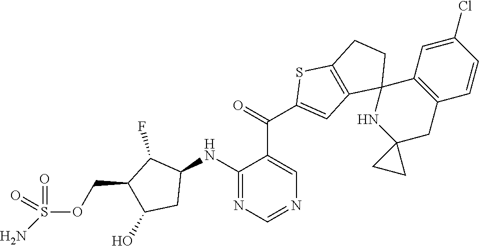 [SMILES: NS(=O)(=O)OC[C@H]1[C@H](F)[C@@H](Nc2ncncc2C(=O)c2cc3c(s2)CCC32NC3(CC3)Cc3ccc(Cl)cc32)C[C@@H]1O] | 634 |
| 167 | 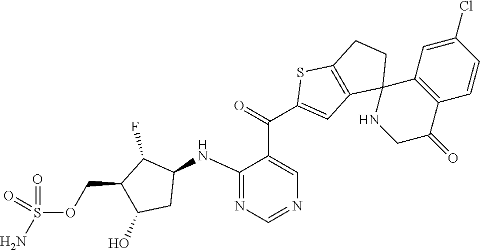 [SMILES: NS(=O)(=O)OC[C@H]1[C@H](F)[C@@H](Nc2ncncc2C(=O)c2cc3c(s2)CCC32NCC(=O)c3ccc(Cl)cc32)C[C@@H]1O] | 622 |
| 168 | 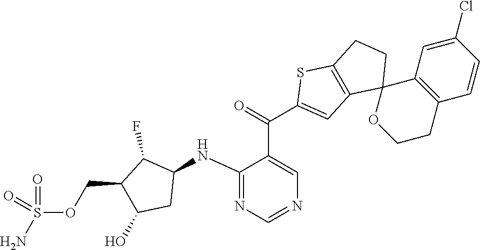 [SMILES: NS(=O)(=O)OC[C@H]1[C@H](F)[C@@H](Nc2ncncc2C(=O)c2cc3c(s2)CCC32OCCc3ccc(Cl)cc32)C[C@@H]1O] | 609 |
| 169 | 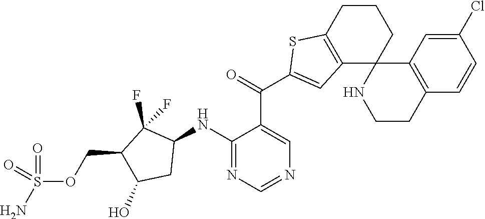 | 640 |
| 170 | 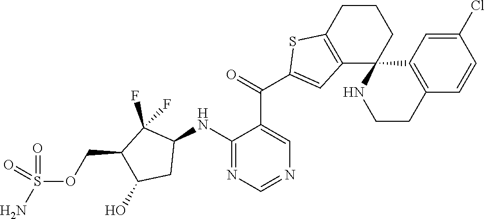 | 640 |
| 171 | 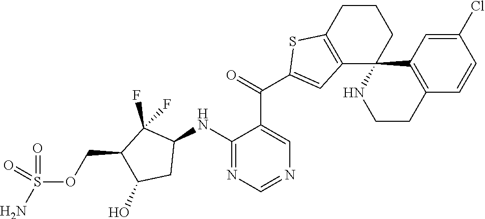 | 640 |
| 172 | 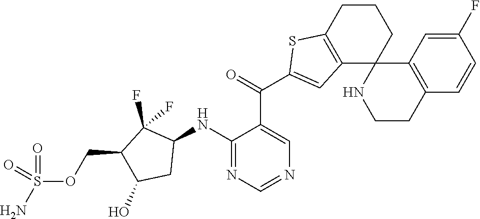 | 624 |
| 173 | 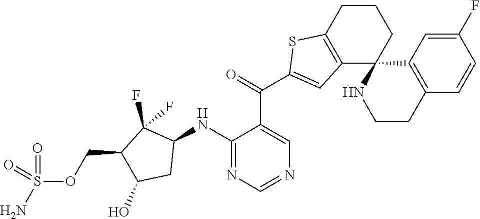 | 624 |
| 174 | 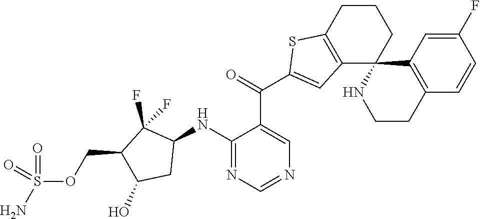 | 624 |
| 175 | 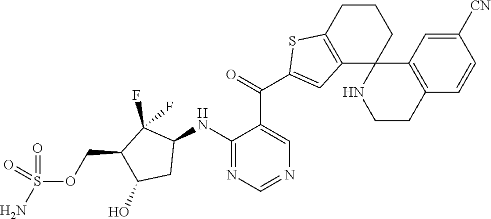 | 631 |
| 176 | 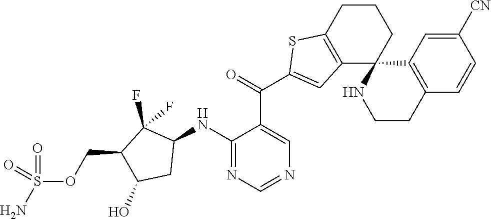 | 631 |
| 177 | 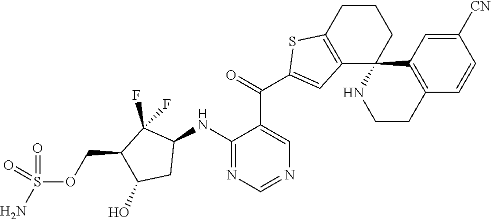 | 631 |
| 178 | 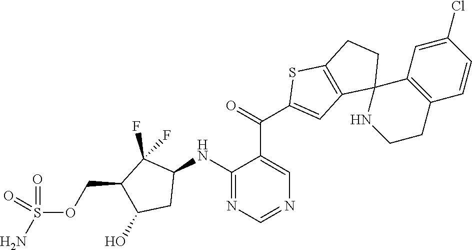 | 626 |
| 179 | 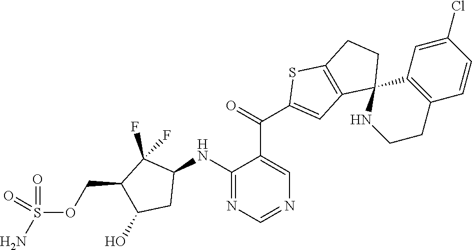 | 626 |
| 180 | 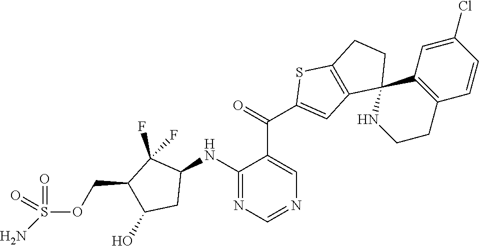 | 626 |
| 181 | 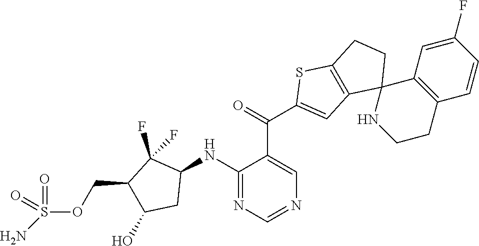 | 610 |
| 182 | 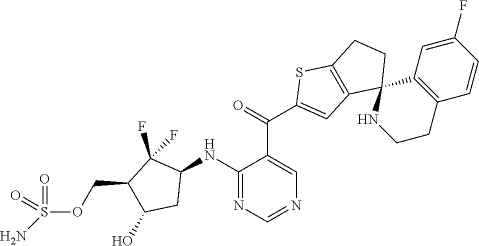 | 610 |
| 183 | 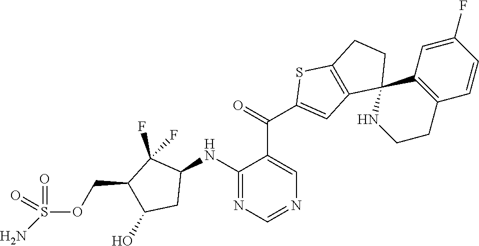 | 610 |
| 184 | 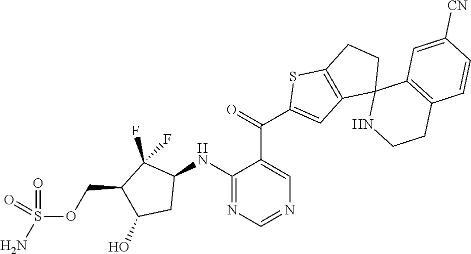 | 617 |
| 185 | 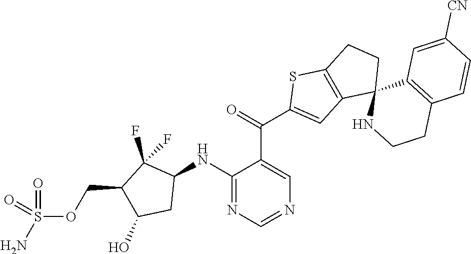 | 617 |
| 186 | 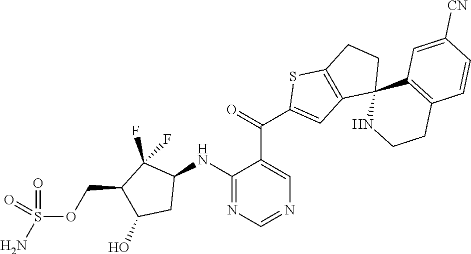 | 617 |
| 187 | 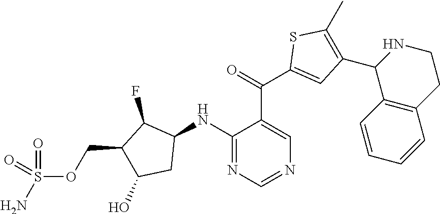 [SMILES: Cc1sc(C(=O)c2cncnc2N[C@H]2C[C@H](O)[C@@H](COS(N)(=O)=O)[C@H]2F)cc1C1NCCc2ccccc21] | 562 |
| 188 | 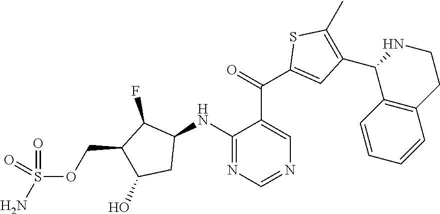 [SMILES: Cc1sc(C(=O)c2cncnc2N[C@H]2C[C@H](O)[C@@H](COS(N)(=O)=O)[C@H]2F)cc1[C@@H]1NCCc2ccccc21] | 562 |
| 189 | 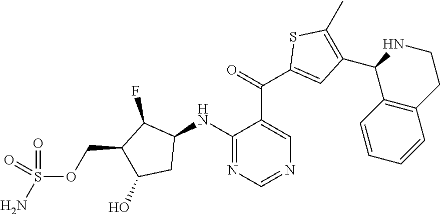 [SMILES: Cc1sc(C(=O)c2cncnc2N[C@H]2C[C@H](O)[C@@H](COS(N)(=O)=O)[C@H]2F)cc1[C@H]1NCCc2ccccc21] | 562 |
| 190 | 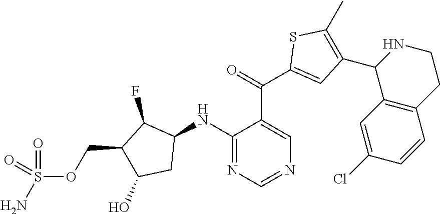 [SMILES: Cc1sc(C(=O)c2cncnc2N[C@H]2C[C@H](O)[C@@H](COS(N)(=O)=O)[C@H]2F)cc1C1NCCc2ccc(Cl)cc21] | 596 |
| 191 | 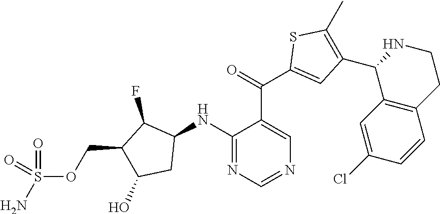 [SMILES: Cc1sc(C(=O)c2cncnc2N[C@H]2C[C@H](O)[C@@H](COS(N)(=O)=O)[C@H]2F)cc1[C@@H]1NCCc2ccc(Cl)cc21] | 596 |
| 192 | 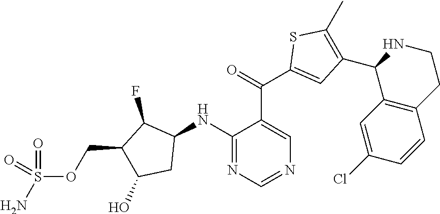 [SMILES: Cc1sc(C(=O)c2cncnc2N[C@H]2C[C@H](O)[C@@H](COS(N)(=O)=O)[C@H]2F)cc1[C@H]1NCCc2ccc(Cl)cc21] | 596 |
| 193 | 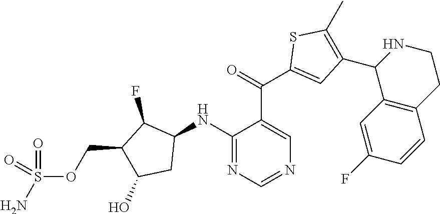 [SMILES: Cc1sc(C(=O)c2cncnc2N[C@H]2C[C@H](O)[C@@H](COS(N)(=O)=O)[C@H]2F)cc1C1NCCc2ccc(F)cc21] | 580 |
| 194 | 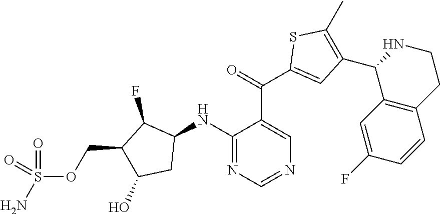 [SMILES: Cc1sc(C(=O)c2cncnc2N[C@H]2C[C@H](O)[C@@H](COS(N)(=O)=O)[C@H]2F)cc1[C@@H]1NCCc2ccc(F)cc21] | 580 |
| 195 | 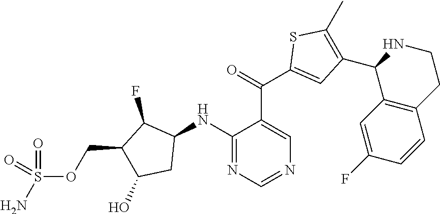 [SMILES: Cc1sc(C(=O)c2cncnc2N[C@H]2C[C@H](O)[C@@H](COS(N)(=O)=O)[C@H]2F)cc1[C@H]1NCCc2ccc(F)cc21] | 580 |
| 196 | 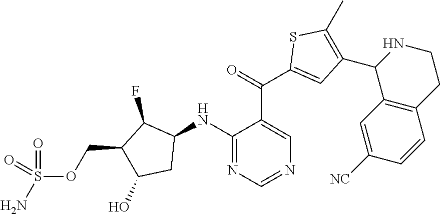 [SMILES: Cc1sc(C(=O)c2cncnc2N[C@H]2C[C@H](O)[C@@H](COS(N)(=O)=O)[C@H]2F)cc1C1NCCc2ccc(C#N)cc21] | 587 |
| 197 | 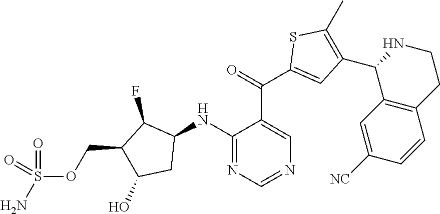 [SMILES: Cc1sc(C(=O)c2cncnc2N[C@H]2C[C@H](O)[C@@H](COS(N)(=O)=O)[C@H]2F)cc1[C@@H]1NCCc2ccc(C#N)cc21] | 587 |
| 198 | 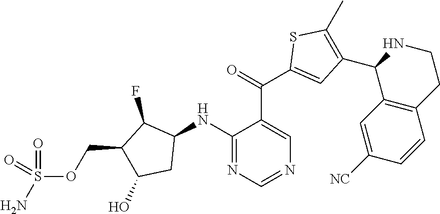 [SMILES: Cc1sc(C(=O)c2cncnc2N[C@H]2C[C@H](O)[C@@H](COS(N)(=O)=O)[C@H]2F)cc1[C@H]1NCCc2ccc(C#N)cc21] | 587 |
| 199 | 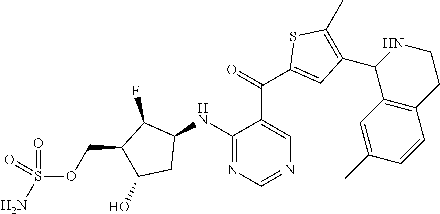 [SMILES: Cc1ccc2c(c1)C(c1cc(C(=O)c3cncnc3N[C@H]3C[C@H](O)[C@@H](COS(N)(=O)=O)[C@H]3F)sc1C)NCC2] | 576 |
| 200 | 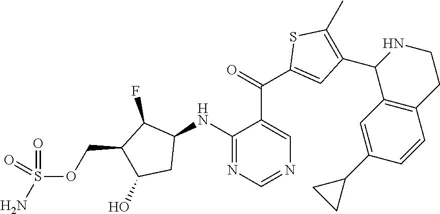 [SMILES: Cc1sc(C(=O)c2cncnc2N[C@H]2C[C@H](O)[C@@H](COS(N)(=O)=O)[C@H]2F)cc1C1NCCc2ccc(C3CC3)cc21] | 602 |
| 201 | 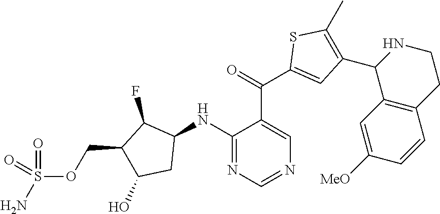 [SMILES: COc1ccc2c(c1)C(c1cc(C(=O)c3cncnc3N[C@H]3C[C@H](O)[C@@H](COS(N)(=O)=O)[C@H]3F)sc1C)NCC2] | 592 |
| 202 | 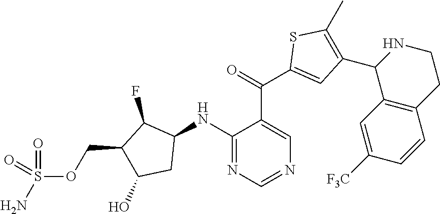 [SMILES: Cc1sc(C(=O)c2cncnc2N[C@H]2C[C@H](O)[C@@H](COS(N)(=O)=O)[C@H]2F)cc1C1NCCc2ccc(C(F)(F)F)cc21] | 630 |
| 203 | 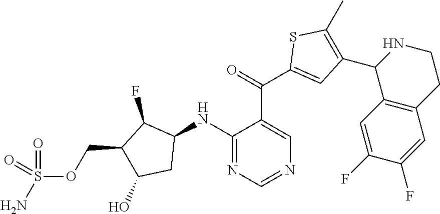 [SMILES: Cc1sc(C(=O)c2cncnc2N[C@H]2C[C@H](O)[C@@H](COS(N)(=O)=O)[C@H]2F)cc1C1NCCc2cc(F)c(F)cc21] | 598 |
| 204 | 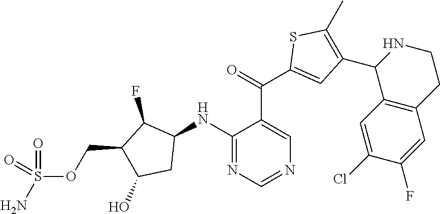 [SMILES: Cc1sc(C(=O)c2cncnc2N[C@H]2C[C@H](O)[C@@H](COS(N)(=O)=O)[C@H]2F)cc1C1NCCc2cc(F)c(Cl)cc21] | 614 |
| 205 | 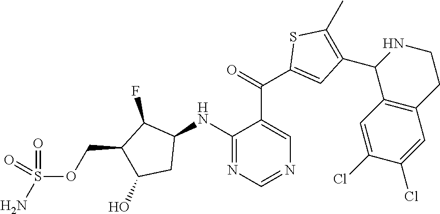 [SMILES: Cc1sc(C(=O)c2cncnc2N[C@H]2C[C@H](O)[C@@H](COS(N)(=O)=O)[C@H]2F)cc1C1NCCc2cc(Cl)c(Cl)cc21] | 630 |
| 206 | 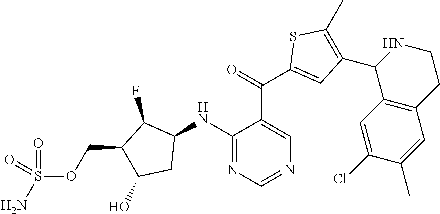 [SMILES: Cc1cc2c(cc1Cl)C(c1cc(C(=O)c3cncnc3N[C@H]3C[C@H](O)[C@@H](COS(N)(=O)=O)[C@H]3F)sc1C)NCC2] | 610 |
| 207 | 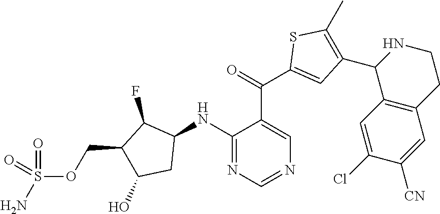 [SMILES: Cc1sc(C(=O)c2cncnc2N[C@H]2C[C@H](O)[C@@H](COS(N)(=O)=O)[C@H]2F)cc1C1NCCc2cc(C#N)c(Cl)cc21] | 621 |
| 208 | 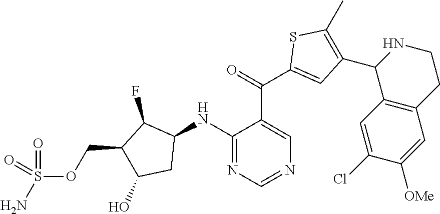 [SMILES: COc1cc2c(cc1Cl)C(c1cc(C(=O)c3cncnc3N[C@H]3C[C@H](O)[C@@H](COS(N)(=O)=O)[C@H]3F)sc1C)NCC2] | 626 |
| 209 | 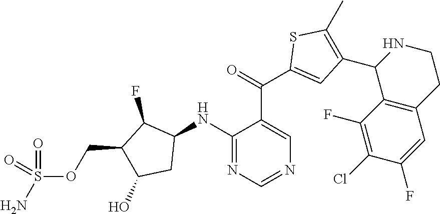 [SMILES: Cc1sc(C(=O)c2cncnc2N[C@H]2C[C@H](O)[C@@H](COS(N)(=O)=O)[C@H]2F)cc1C1NCCc2cc(F)c(Cl)c(F)c21] | 632 |
| 210 | 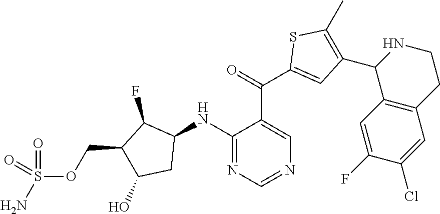 [SMILES: Cc1sc(C(=O)c2cncnc2N[C@H]2C[C@H](O)[C@@H](COS(N)(=O)=O)[C@H]2F)cc1C1NCCc2cc(Cl)c(F)cc21] | 614 |
| 211 | 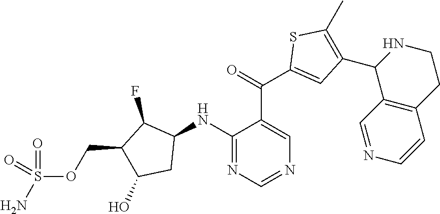 [SMILES: Cc1sc(C(=O)c2cncnc2N[C@H]2C[C@H](O)[C@@H](COS(N)(=O)=O)[C@H]2F)cc1C1NCCc2ccncc21] | 563 |
| 212 | 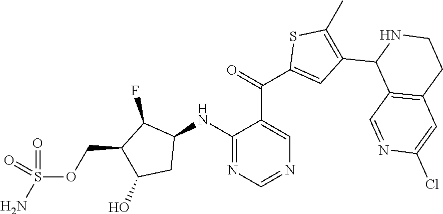 [SMILES: Cc1sc(C(=O)c2cncnc2N[C@H]2C[C@H](O)[C@@H](COS(N)(=O)=O)[C@H]2F)cc1C1NCCc2cc(Cl)ncc21] | 597 |
| 213 | 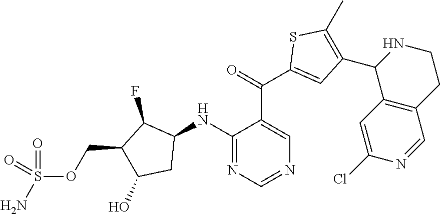 [SMILES: Cc1sc(C(=O)c2cncnc2N[C@H]2C[C@H](O)[C@@H](COS(N)(=O)=O)[C@H]2F)cc1C1NCCc2cnc(Cl)cc21] | 597 |
| 214 | 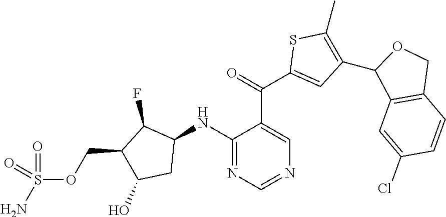 [SMILES: Cc1sc(C(=O)c2cncnc2N[C@H]2C[C@H](O)[C@@H](COS(N)(=O)=O)[C@H]2F)cc1C1OCc2ccc(Cl)cc21] | 583 |
| 215 | 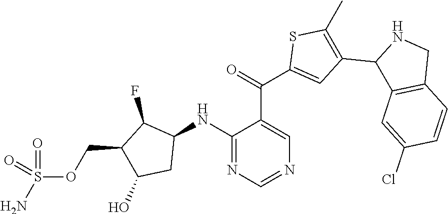 [SMILES: Cc1sc(C(=O)c2cncnc2N[C@H]2C[C@H](O)[C@@H](COS(N)(=O)=O)[C@H]2F)cc1C1NCc2ccc(Cl)cc21] | 582 |
| 216 | 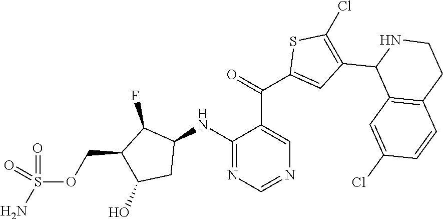 [SMILES: NS(=O)(=O)OC[C@H]1[C@@H](F)[C@@H](Nc2ncncc2C(=O)c2cc(C3NCCc4ccc(Cl)cc43)c(Cl)s2)C[C@@H]1O] | 616 |
| 217 | 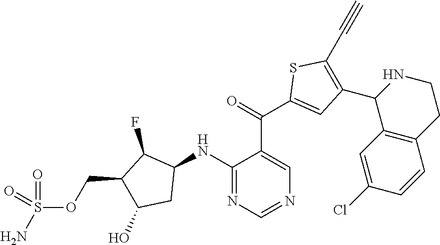 [SMILES: C#Cc1sc(C(=O)c2cncnc2N[C@H]2C[C@H](O)[C@@H](COS(N)(=O)=O)[C@H]2F)cc1C1NCCc2ccc(Cl)cc21] | 606 |
| 218 | 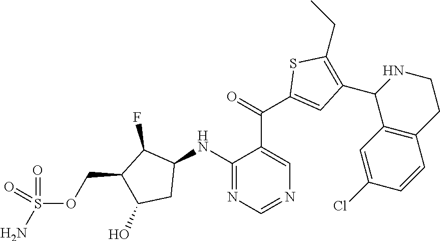 [SMILES: CCc1sc(C(=O)c2cncnc2N[C@H]2C[C@H](O)[C@@H](COS(N)(=O)=O)[C@H]2F)cc1C1NCCc2ccc(Cl)cc21] | 610 |
| 219 | 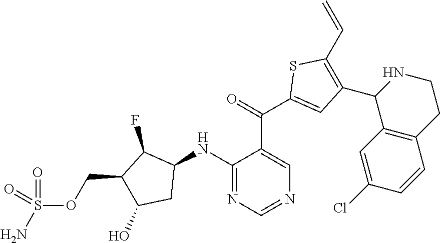 [SMILES: C=Cc1sc(C(=O)c2cncnc2N[C@H]2C[C@H](O)[C@@H](COS(N)(=O)=O)[C@H]2F)cc1C1NCCc2ccc(Cl)cc21] | 608 |
| 220 | 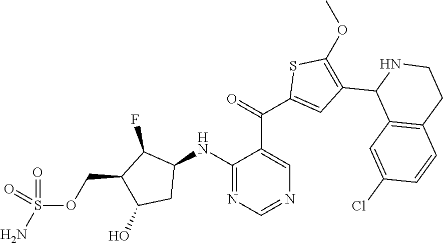 [SMILES: COc1sc(C(=O)c2cncnc2N[C@H]2C[C@H](O)[C@@H](COS(N)(=O)=O)[C@H]2F)cc1C1NCCc2ccc(Cl)cc21] | 612 |
| 221 | 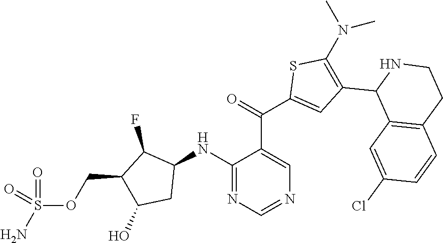 [SMILES: CN(C)c1sc(C(=O)c2cncnc2N[C@H]2C[C@H](O)[C@@H](COS(N)(=O)=O)[C@H]2F)cc1C1NCCc2ccc(Cl)cc21] | 625 |
| 222 | 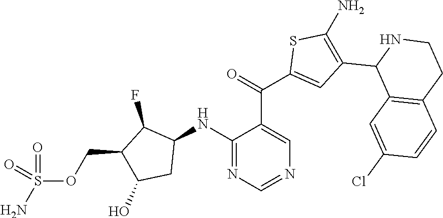 [SMILES: Nc1sc(C(=O)c2cncnc2N[C@H]2C[C@H](O)[C@@H](COS(N)(=O)=O)[C@H]2F)cc1C1NCCc2ccc(Cl)cc21] | 597 |
| 223 | 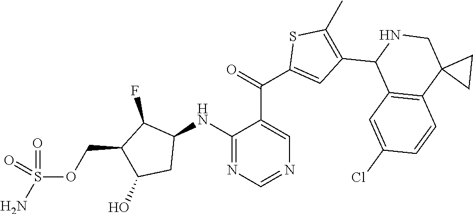 [SMILES: Cc1sc(C(=O)c2cncnc2N[C@H]2C[C@H](O)[C@@H](COS(N)(=O)=O)[C@H]2F)cc1C1NCC2(CC2)c2ccc(Cl)cc21] | 622 |
| 224 | 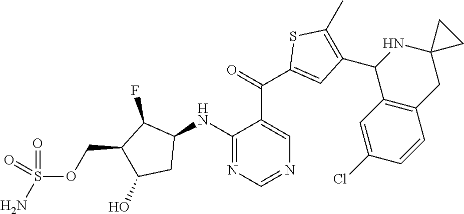 [SMILES: Cc1sc(C(=O)c2cncnc2N[C@H]2C[C@H](O)[C@@H](COS(N)(=O)=O)[C@H]2F)cc1C1NC2(CC2)Cc2ccc(Cl)cc21] | 622 |
| 225 | 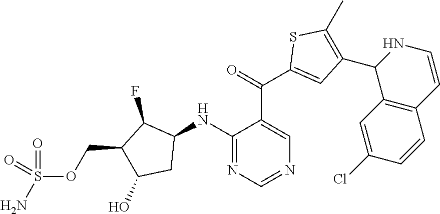 [SMILES: Cc1sc(C(=O)c2cncnc2N[C@H]2C[C@H](O)[C@@H](COS(N)(=O)=O)[C@H]2F)cc1C1NC=Cc2ccc(Cl)cc21] | 594 |
| 226 | 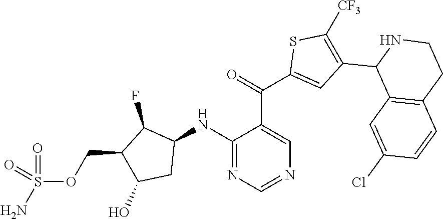 [SMILES: NS(=O)(=O)OC[C@H]1[C@@H](F)[C@@H](Nc2ncncc2C(=O)c2cc(C3NCCc4ccc(Cl)cc43)c(C(F)(F)F)s2)C[C@@H]1O] | 650 |
| 227 | 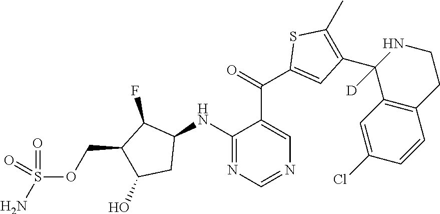 [SMILES: [2H]C1(c2cc(C(=O)c3cncnc3N[C@H]3C[C@H](O)[C@@H](COS(N)(=O)=O)[C@H]3F)sc2C)NCCc2ccc(Cl)cc21] | 597 |
| 228 | 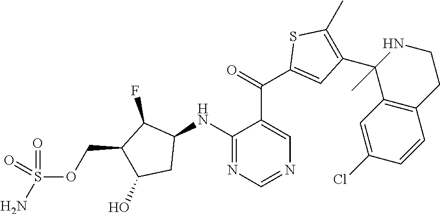 [SMILES: Cc1sc(C(=O)c2cncnc2N[C@H]2C[C@H](O)[C@@H](COS(N)(=O)=O)[C@H]2F)cc1C1(C)NCCc2ccc(Cl)cc21] | 610 |
| 229 | 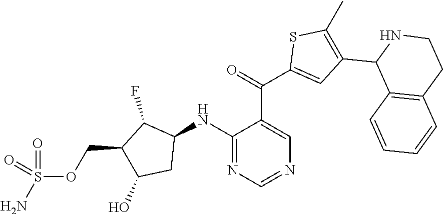 [SMILES: Cc1sc(C(=O)c2cncnc2N[C@H]2C[C@H](O)[C@@H](COS(N)(=O)=O)[C@@H]2F)cc1C1NCCc2ccccc21] | 562 |
| 230 | 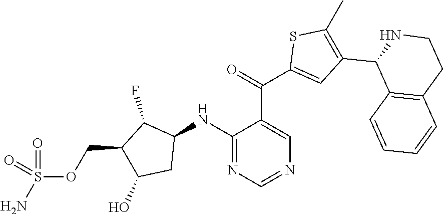 [SMILES: Cc1sc(C(=O)c2cncnc2N[C@H]2C[C@H](O)[C@@H](COS(N)(=O)=O)[C@@H]2F)cc1[C@@H]1NCCc2ccccc21] | 562 |
| 231 | 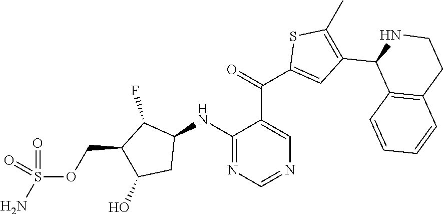 [SMILES: Cc1sc(C(=O)c2cncnc2N[C@H]2C[C@H](O)[C@@H](COS(N)(=O)=O)[C@@H]2F)cc1[C@H]1NCCc2ccccc21] | 562 |
| 232 | 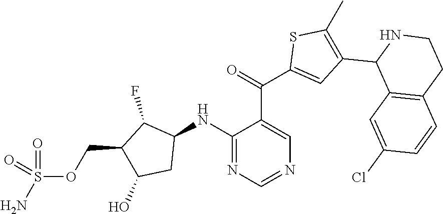 [SMILES: Cc1sc(C(=O)c2cncnc2N[C@H]2C[C@H](O)[C@@H](COS(N)(=O)=O)[C@@H]2F)cc1C1NCCc2ccc(Cl)cc21] | 596 |
| 233 | 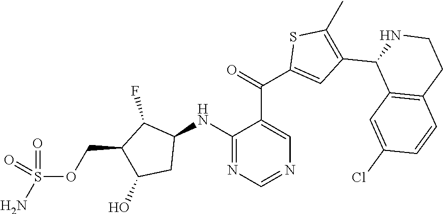 [SMILES: Cc1sc(C(=O)c2cncnc2N[C@H]2C[C@H](O)[C@@H](COS(N)(=O)=O)[C@@H]2F)cc1[C@@H]1NCCc2ccc(Cl)cc21] | 596 |
| 234 | 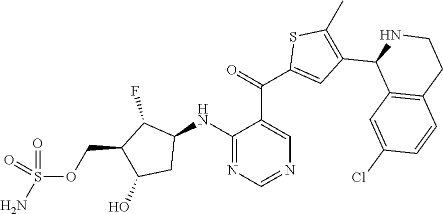 [SMILES: Cc1sc(C(=O)c2cncnc2N[C@H]2C[C@H](O)[C@@H](COS(N)(=O)=O)[C@@H]2F)cc1[C@H]1NCCc2ccc(Cl)cc21] | 596 |
| 235 | 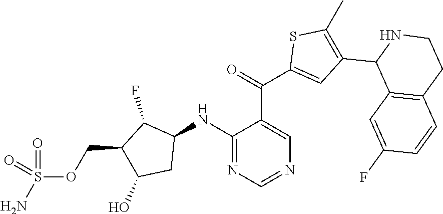 [SMILES: Cc1sc(C(=O)c2cncnc2N[C@H]2C[C@H](O)[C@@H](COS(N)(=O)=O)[C@@H]2F)cc1C1NCCc2ccc(F)cc21] | 580 |
| 236 | 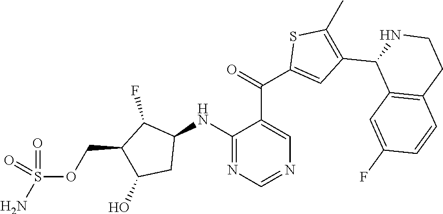 [SMILES: Cc1sc(C(=O)c2cncnc2N[C@H]2C[C@H](O)[C@@H](COS(N)(=O)=O)[C@@H]2F)cc1[C@@H]1NCCc2ccc(F)cc21] | 580 |
| 237 | 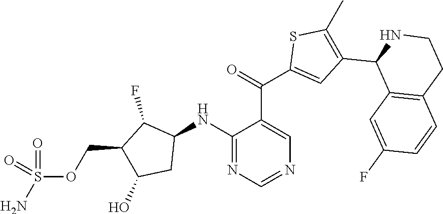 [SMILES: Cc1sc(C(=O)c2cncnc2N[C@H]2C[C@H](O)[C@@H](COS(N)(=O)=O)[C@@H]2F)cc1[C@H]1NCCc2ccc(F)cc21] | 580 |
| 238 | 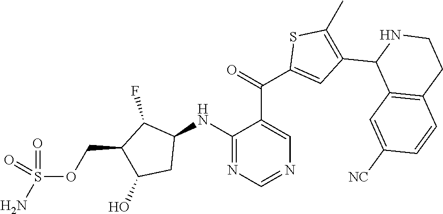 [SMILES: Cc1sc(C(=O)c2cncnc2N[C@H]2C[C@H](O)[C@@H](COS(N)(=O)=O)[C@@H]2F)cc1C1NCCc2ccc(C#N)cc21] | 587 |
| 239 | 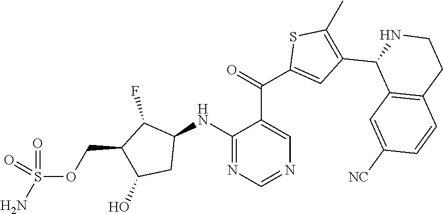 [SMILES: Cc1sc(C(=O)c2cncnc2N[C@H]2C[C@H](O)[C@@H](COS(N)(=O)=O)[C@@H]2F)cc1[C@@H]1NCCc2ccc(C#N)cc21] | 587 |
| 240 | 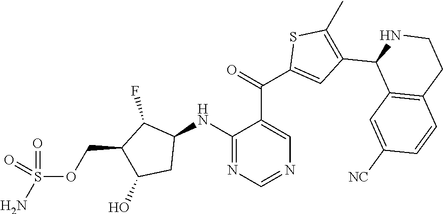 [SMILES: Cc1sc(C(=O)c2cncnc2N[C@H]2C[C@H](O)[C@@H](COS(N)(=O)=O)[C@@H]2F)cc1[C@H]1NCCc2ccc(C#N)cc21] | 587 |
| 241 | 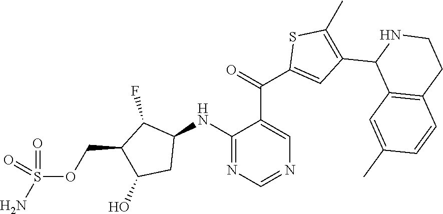 [SMILES: Cc1ccc2c(c1)C(c1cc(C(=O)c3cncnc3N[C@H]3C[C@H](O)[C@@H](COS(N)(=O)=O)[C@@H]3F)sc1C)NCC2] | 576 |
| 242 | 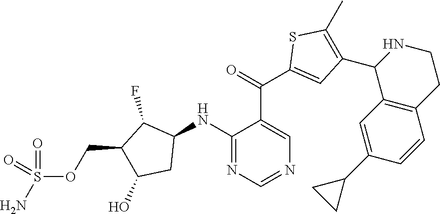 [SMILES: Cc1sc(C(=O)c2cncnc2N[C@H]2C[C@H](O)[C@@H](COS(N)(=O)=O)[C@@H]2F)cc1C1NCCc2ccc(C3CC3)cc21] | 602 |
| 243 | 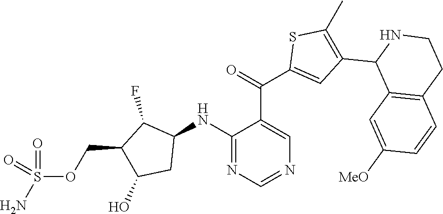 [SMILES: COc1ccc2c(c1)C(c1cc(C(=O)c3cncnc3N[C@H]3C[C@H](O)[C@@H](COS(N)(=O)=O)[C@@H]3F)sc1C)NCC2] | 592 |
| 244 | 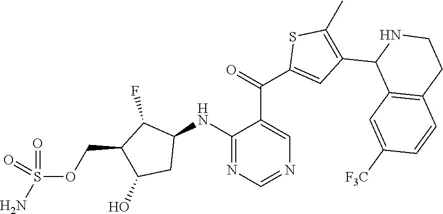 [SMILES: Cc1sc(C(=O)c2cncnc2N[C@H]2C[C@H](O)[C@@H](COS(N)(=O)=O)[C@@H]2F)cc1C1NCCc2ccc(C(F)(F)F)cc21] | 630 |
| 245 | 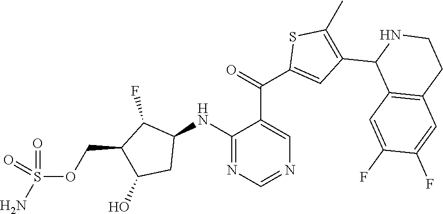 [SMILES: Cc1sc(C(=O)c2cncnc2N[C@H]2C[C@H](O)[C@@H](COS(N)(=O)=O)[C@@H]2F)cc1C1NCCc2cc(F)c(F)cc21] | 598 |
| 246 | 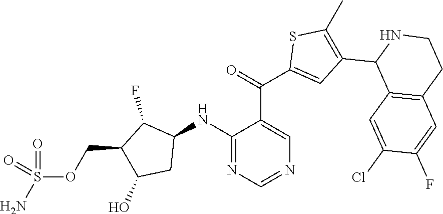 [SMILES: Cc1sc(C(=O)c2cncnc2N[C@H]2C[C@H](O)[C@@H](COS(N)(=O)=O)[C@@H]2F)cc1C1NCCc2cc(F)c(Cl)cc21] | 614 |
| 247 | 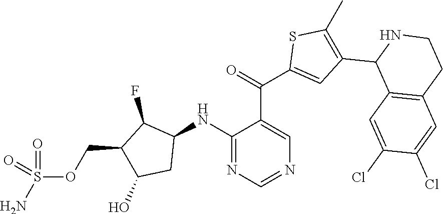 [SMILES: Cc1sc(C(=O)c2cncnc2N[C@H]2C[C@H](O)[C@@H](COS(N)(=O)=O)[C@H]2F)cc1C1NCCc2cc(Cl)c(Cl)cc21] | 630 |
| 248 | 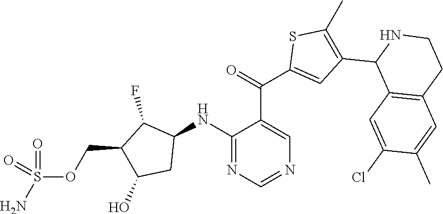 [SMILES: Cc1cc2c(cc1Cl)C(c1cc(C(=O)c3cncnc3N[C@H]3C[C@H](O)[C@@H](COS(N)(=O)=O)[C@@H]3F)sc1C)NCC2] | 610 |
| 249 | 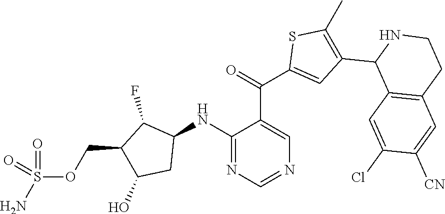 [SMILES: Cc1sc(C(=O)c2cncnc2N[C@H]2C[C@H](O)[C@@H](COS(N)(=O)=O)[C@@H]2F)cc1C1NCCc2cc(C#N)c(Cl)cc21] | 621 |
| 250 | 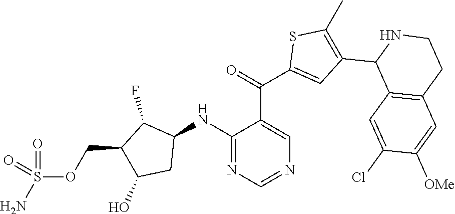 [SMILES: COc1cc2c(cc1Cl)C(c1cc(C(=O)c3cncnc3N[C@H]3C[C@H](O)[C@@H](COS(N)(=O)=O)[C@@H]3F)sc1C)NCC2] | 626 |
| 251 | 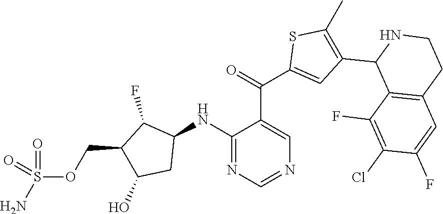 [SMILES: Cc1sc(C(=O)c2cncnc2N[C@H]2C[C@H](O)[C@@H](COS(N)(=O)=O)[C@@H]2F)cc1C1NCCc2cc(F)c(Cl)c(F)c21] | 632 |
| 252 | 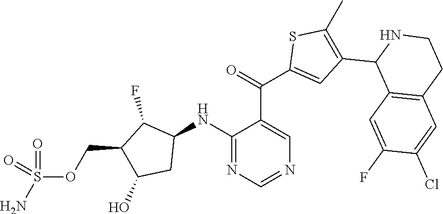 [SMILES: Cc1sc(C(=O)c2cncnc2N[C@H]2C[C@H](O)[C@@H](COS(N)(=O)=O)[C@@H]2F)cc1C1NCCc2cc(Cl)c(F)cc21] | 614 |
| 253 | 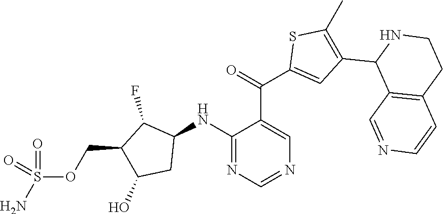 [SMILES: Cc1sc(C(=O)c2cncnc2N[C@H]2C[C@H](O)[C@@H](COS(N)(=O)=O)[C@@H]2F)cc1C1NCCc2ccncc21] | 563 |
| 254 | 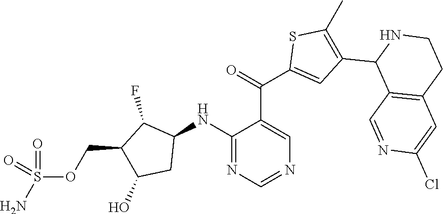 [SMILES: Cc1sc(C(=O)c2cncnc2N[C@H]2C[C@H](O)[C@@H](COS(N)(=O)=O)[C@@H]2F)cc1C1NCCc2cc(Cl)ncc21] | 597 |
| 255 | 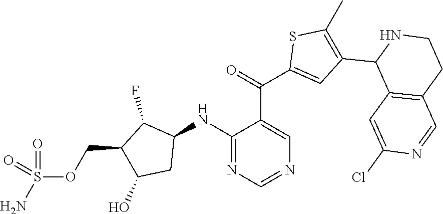 [SMILES: Cc1sc(C(=O)c2cncnc2N[C@H]2C[C@H](O)[C@@H](COS(N)(=O)=O)[C@@H]2F)cc1C1NCCc2cnc(Cl)cc21] | 597 |
| 256 | 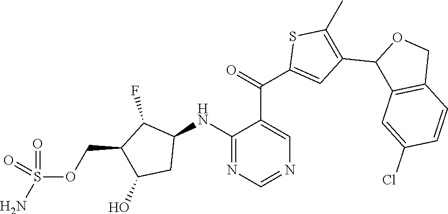 [SMILES: Cc1sc(C(=O)c2cncnc2N[C@H]2C[C@H](O)[C@@H](COS(N)(=O)=O)[C@@H]2F)cc1C1OCc2ccc(Cl)cc21] | 583 |
| 257 | 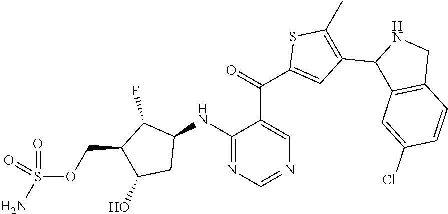 [SMILES: Cc1sc(C(=O)c2cncnc2N[C@H]2C[C@H](O)[C@@H](COS(N)(=O)=O)[C@@H]2F)cc1C1NCc2ccc(Cl)cc21] | 582 |
| 258 | 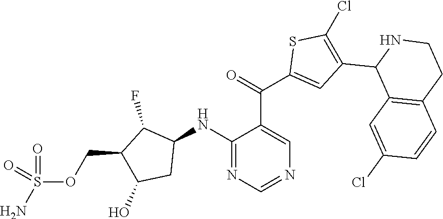 [SMILES: NS(=O)(=O)OC[C@H]1[C@H](F)[C@@H](Nc2ncncc2C(=O)c2cc(C3NCCc4ccc(Cl)cc43)c(Cl)s2)C[C@@H]1O] | 616 |
| 259 | 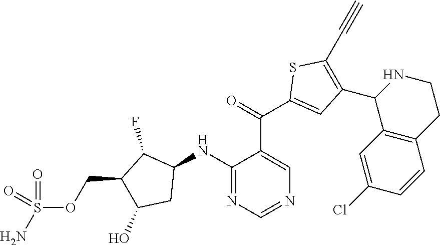 [SMILES: C#Cc1sc(C(=O)c2cncnc2N[C@H]2C[C@H](O)[C@@H](COS(N)(=O)=O)[C@@H]2F)cc1C1NCCc2ccc(Cl)cc21] | 606 |
| 260 | 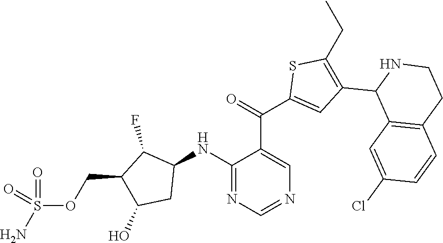 [SMILES: CCc1sc(C(=O)c2cncnc2N[C@H]2C[C@H](O)[C@@H](COS(N)(=O)=O)[C@@H]2F)cc1C1NCCc2ccc(Cl)cc21] | 610 |
| 261 | 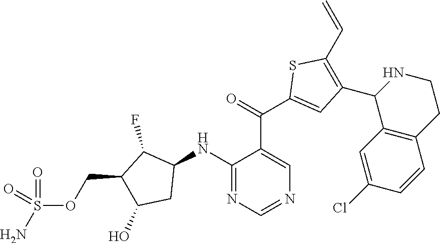 [SMILES: C=Cc1sc(C(=O)c2cncnc2N[C@H]2C[C@H](O)[C@@H](COS(N)(=O)=O)[C@@H]2F)cc1C1NCCc2ccc(Cl)cc21] | 608 |
| 262 | 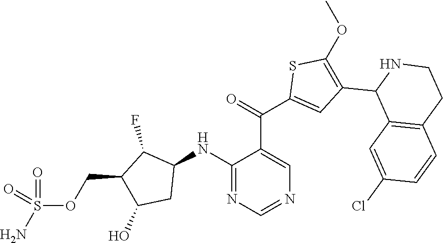 [SMILES: COc1sc(C(=O)c2cncnc2N[C@H]2C[C@H](O)[C@@H](COS(N)(=O)=O)[C@@H]2F)cc1C1NCCc2ccc(Cl)cc21] | 612 |
| 263 | 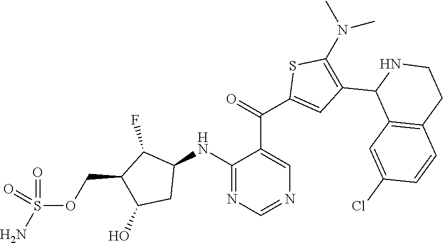 [SMILES: CN(C)c1sc(C(=O)c2cncnc2N[C@H]2C[C@H](O)[C@@H](COS(N)(=O)=O)[C@@H]2F)cc1C1NCCc2ccc(Cl)cc21] | 625 |
| 264 | 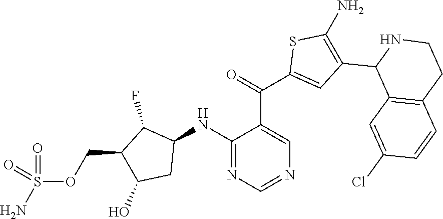 [SMILES: Nc1sc(C(=O)c2cncnc2N[C@H]2C[C@H](O)[C@@H](COS(N)(=O)=O)[C@@H]2F)cc1C1NCCc2ccc(Cl)cc21] | 597 |
| 265 | 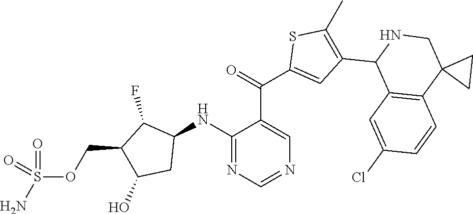 [SMILES: Cc1sc(C(=O)c2cncnc2N[C@H]2C[C@H](O)[C@@H](COS(N)(=O)=O)[C@@H]2F)cc1C1NCC2(CC2)c2ccc(Cl)cc21] | 622 |
| 266 | 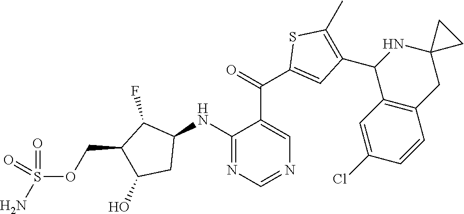 [SMILES: Cc1sc(C(=O)c2cncnc2N[C@H]2C[C@H](O)[C@@H](COS(N)(=O)=O)[C@@H]2F)cc1C1NC2(CC2)Cc2ccc(Cl)cc21] | 622 |
| 267 | 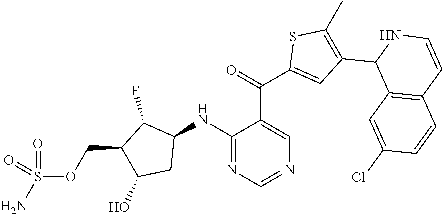 [SMILES: Cc1sc(C(=O)c2cncnc2N[C@H]2C[C@H](O)[C@@H](COS(N)(=O)=O)[C@@H]2F)cc1C1NC=Cc2ccc(Cl)cc21] | 594 |
| 268 | 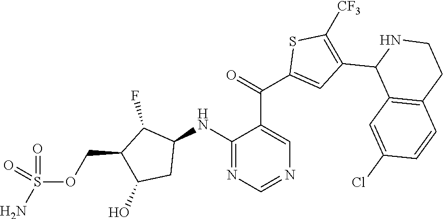 [SMILES: NS(=O)(=O)OC[C@H]1[C@H](F)[C@@H](Nc2ncncc2C(=O)c2cc(C3NCCc4ccc(Cl)cc43)c(C(F)(F)F)s2)C[C@@H]1O] | 650 |
| 269 | 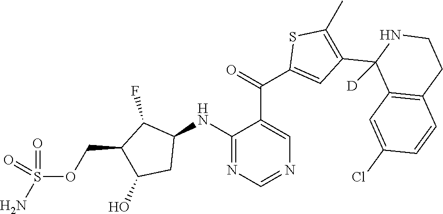 [SMILES: [2H]C1(c2cc(C(=O)c3cncnc3N[C@H]3C[C@H](O)[C@@H](COS(N)(=O)=O)[C@@H]3F)sc2C)NCCc2ccc(Cl)cc21] | 597 |
| 270 | 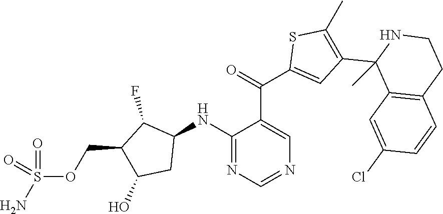 [SMILES: Cc1sc(C(=O)c2cncnc2N[C@H]2C[C@H](O)[C@@H](COS(N)(=O)=O)[C@@H]2F)cc1C1(C)NCCc2ccc(Cl)cc21] | 610 |
| 271 | 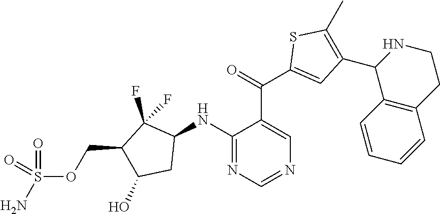 | 580 |
| 272 | 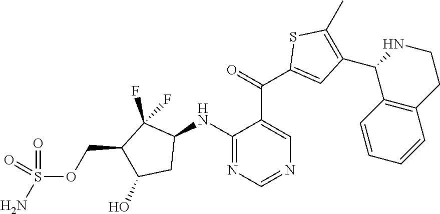 | 580 |
| 273 | 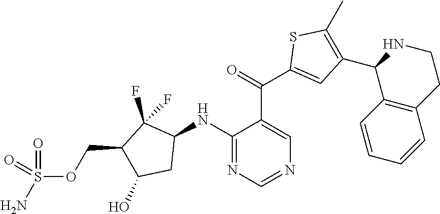 | 580 |
| 274 | 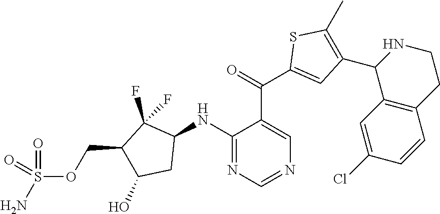 | 614 |
| 275 | 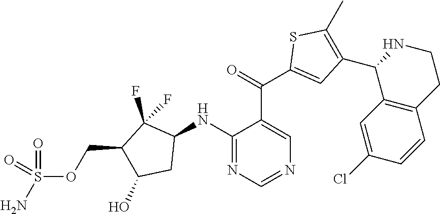 | 614 |
| 276 | 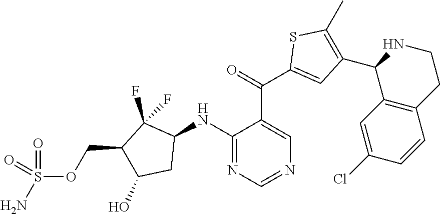 | 614 |
| 277 | 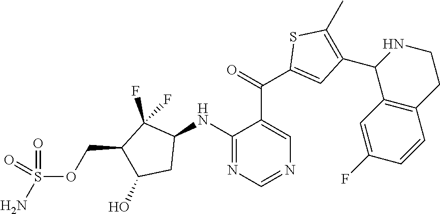 | 598 |
| 278 | 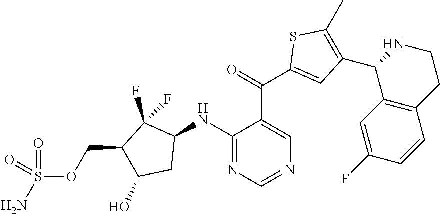 | 598 |
| 279 | 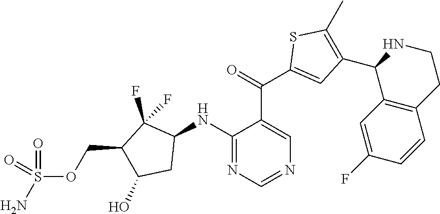 | 598 |
| 280 | 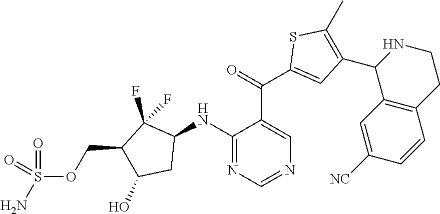 | 605 |
| 281 | 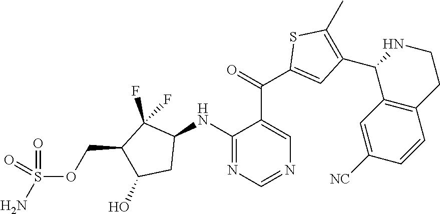 | 605 |
| 282 | 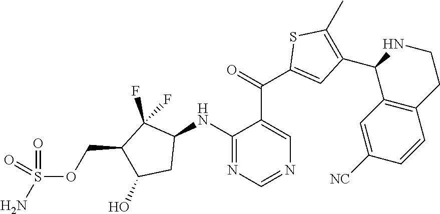 | 605 |
| 283 | 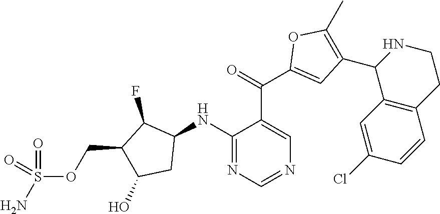 [SMILES: Cc1oc(C(=O)c2cncnc2N[C@H]2C[C@H](O)[C@@H](COS(N)(=O)=O)[C@H]2F)cc1C1NCCc2ccc(Cl)cc21] | 580 |
| 284 | 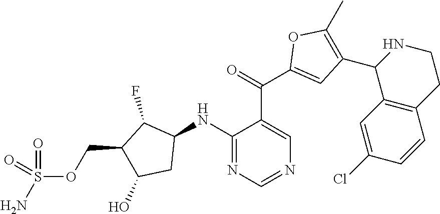 [SMILES: Cc1oc(C(=O)c2cncnc2N[C@H]2C[C@H](O)[C@@H](COS(N)(=O)=O)[C@@H]2F)cc1C1NCCc2ccc(Cl)cc21] | 580 |
| 285 | 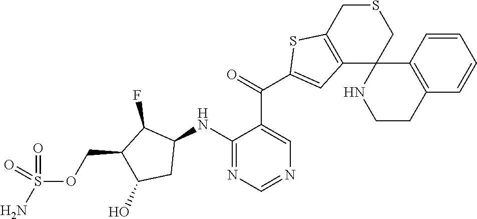 [SMILES: NS(=O)(=O)OC[C@H]1[C@@H](F)[C@@H](Nc2ncncc2C(=O)c2cc3c(s2)CSCC32NCCc3ccccc32)C[C@@H]1O] | 606 |
| 286 | 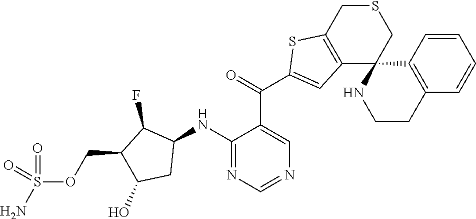 [SMILES: NS(=O)(=O)OC[C@H]1[C@@H](F)[C@@H](Nc2ncncc2C(=O)c2cc3c(s2)CSC[C@@]32NCCc3ccccc32)C[C@@H]1O] | 606 |
| 287 | 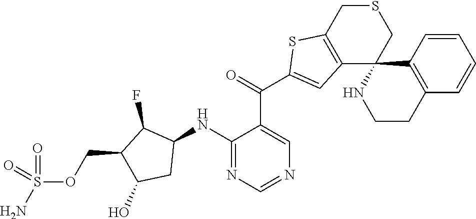 [SMILES: NS(=O)(=O)OC[C@H]1[C@@H](F)[C@@H](Nc2ncncc2C(=O)c2cc3c(s2)CSC[C@]32NCCc3ccccc32)C[C@@H]1O] | 606 |
| 288 | 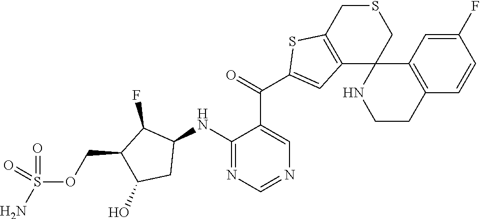 [SMILES: NS(=O)(=O)OC[C@H]1[C@@H](F)[C@@H](Nc2ncncc2C(=O)c2cc3c(s2)CSCC32NCCc3ccc(F)cc32)C[C@@H]1O] | 624 |
| 289 | 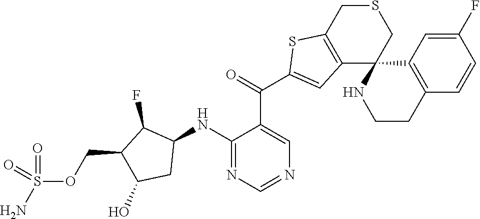 [SMILES: NS(=O)(=O)OC[C@H]1[C@@H](F)[C@@H](Nc2ncncc2C(=O)c2cc3c(s2)CSC[C@@]32NCCc3ccc(F)cc32)C[C@@H]1O] | 624 |
| 290 | 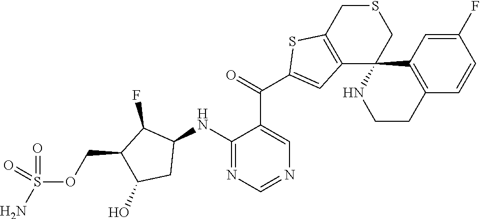 [SMILES: NS(=O)(=O)OC[C@H]1[C@@H](F)[C@@H](Nc2ncncc2C(=O)c2cc3c(s2)CSC[C@]32NCCc3ccc(F)cc32)C[C@@H]1O] | 624 |
| 291 | 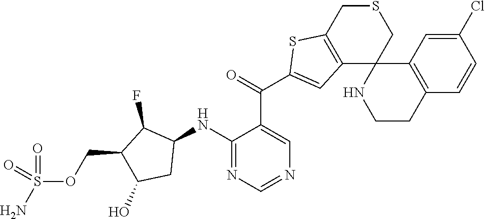 [SMILES: NS(=O)(=O)OC[C@H]1[C@@H](F)[C@@H](Nc2ncncc2C(=O)c2cc3c(s2)CSCC32NCCc3ccc(Cl)cc32)C[C@@H]1O] | 640 |
| 292 | 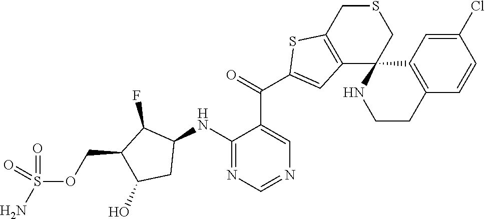 [SMILES: NS(=O)(=O)OC[C@H]1[C@@H](F)[C@@H](Nc2ncncc2C(=O)c2cc3c(s2)CSC[C@@]32NCCc3ccc(Cl)cc32)C[C@@H]1O] | 640 |
| 293 | 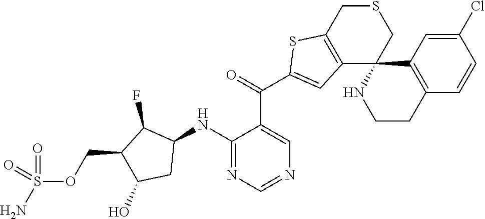 [SMILES: NS(=O)(=O)OC[C@H]1[C@@H](F)[C@@H](Nc2ncncc2C(=O)c2cc3c(s2)CSC[C@]32NCCc3ccc(Cl)cc32)C[C@@H]1O] | 640 |
| 294 | 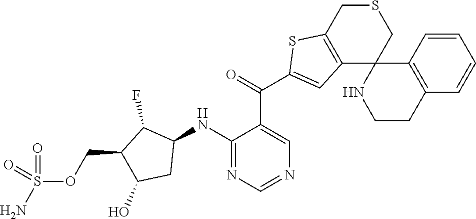 [SMILES: NS(=O)(=O)OC[C@H]1[C@H](F)[C@@H](Nc2ncncc2C(=O)c2cc3c(s2)CSCC32NCCc3ccccc32)C[C@@H]1O] | 606 |
| 295 | 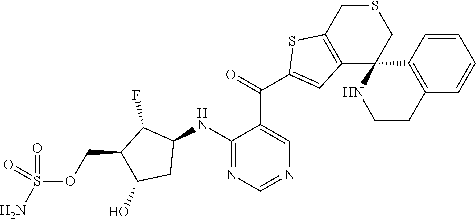 [SMILES: NS(=O)(=O)OC[C@H]1[C@H](F)[C@@H](Nc2ncncc2C(=O)c2cc3c(s2)CSC[C@@]32NCCc3ccccc32)C[C@@H]1O] | 606 |
| 296 | 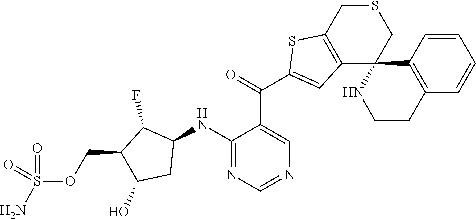 [SMILES: NS(=O)(=O)OC[C@H]1[C@H](F)[C@@H](Nc2ncncc2C(=O)c2cc3c(s2)CSC[C@]32NCCc3ccccc32)C[C@@H]1O] | 606 |
| 297 | 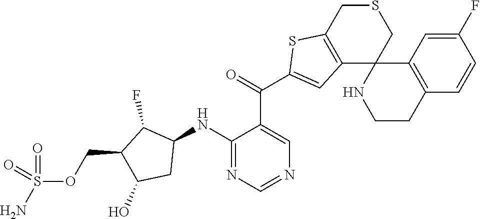 [SMILES: NS(=O)(=O)OC[C@H]1[C@H](F)[C@@H](Nc2ncncc2C(=O)c2cc3c(s2)CSCC32NCCc3ccc(F)cc32)C[C@@H]1O] | 624 |
| 298 | 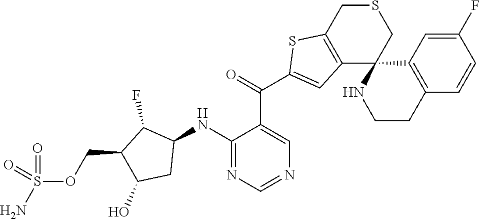 [SMILES: NS(=O)(=O)OC[C@H]1[C@H](F)[C@@H](Nc2ncncc2C(=O)c2cc3c(s2)CSC[C@@]32NCCc3ccc(F)cc32)C[C@@H]1O] | 624 |
| 299 | 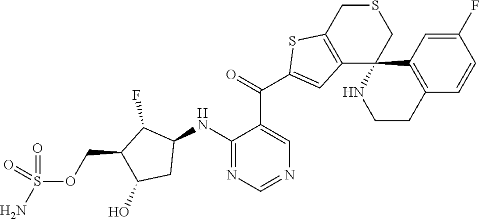 [SMILES: NS(=O)(=O)OC[C@H]1[C@H](F)[C@@H](Nc2ncncc2C(=O)c2cc3c(s2)CSC[C@]32NCCc3ccc(F)cc32)C[C@@H]1O] | 624 |
| 300 | 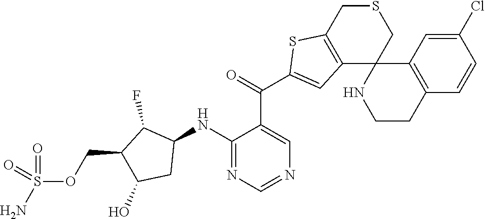 [SMILES: NS(=O)(=O)OC[C@H]1[C@H](F)[C@@H](Nc2ncncc2C(=O)c2cc3c(s2)CSCC32NCCc3ccc(Cl)cc32)C[C@@H]1O] | 640 |
| 301 | 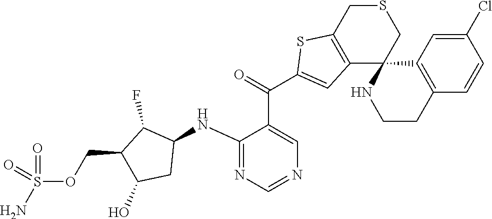 [SMILES: NS(=O)(=O)OC[C@H]1[C@H](F)[C@@H](Nc2ncncc2C(=O)c2cc3c(s2)CSC[C@@]32NCCc3ccc(Cl)cc32)C[C@@H]1O] | 640 |
| 302 | 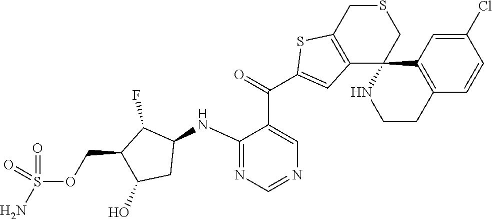 [SMILES: NS(=O)(=O)OC[C@H]1[C@H](F)[C@@H](Nc2ncncc2C(=O)c2cc3c(s2)CSC[C@]32NCCc3ccc(Cl)cc32)C[C@@H]1O] | 640 |
| 303 | 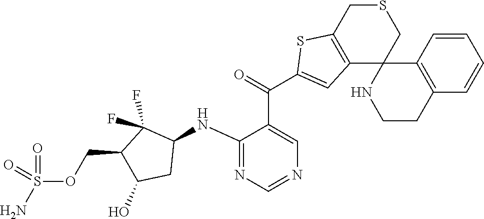 | 624 |
| 304 | 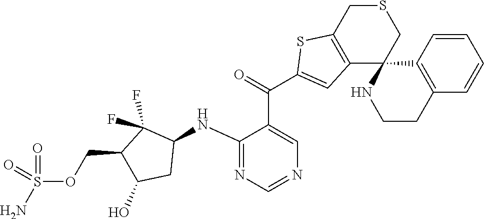 | 624 |
| 305 | 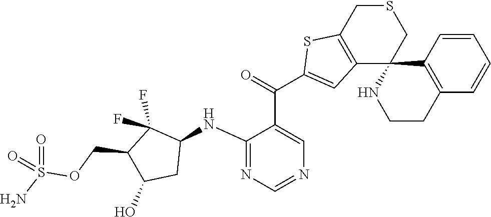 | 624 |
| 306 | 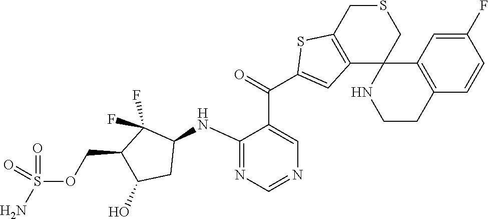 | 642 |
| 307 | 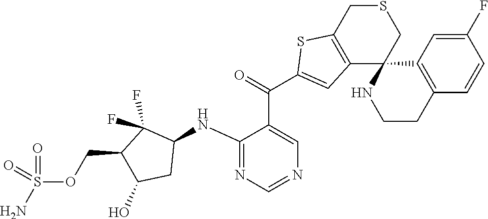 | 642 |
| 308 | 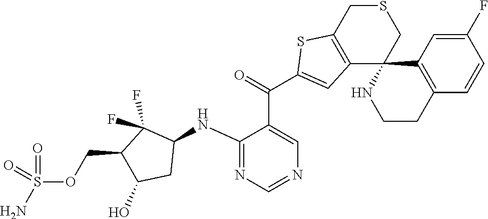 | 642 |
| 309 | 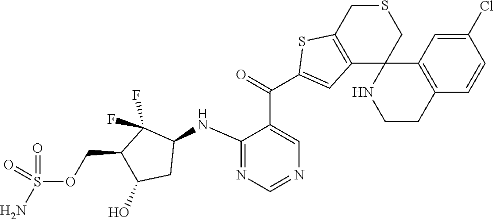 | 658 |
| 310 | 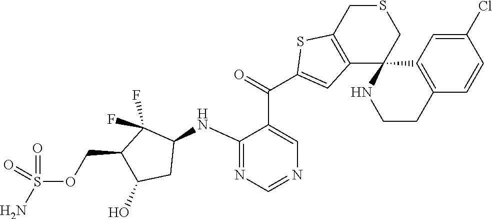 | 658 |
| 311 | 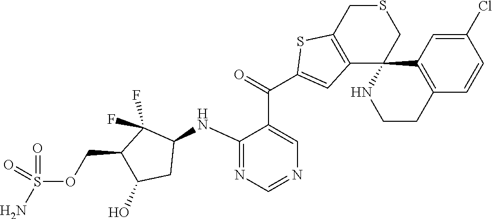 | 658 |
| 312 | 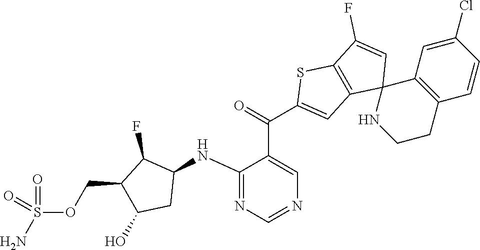 [SMILES: NS(=O)(=O)OC[C@H]1[C@@H](F)[C@@H](Nc2ncncc2C(=O)c2cc3c(s2)C(F)=CC32NCCc3ccc(Cl)cc32)C[C@@H]1O] | 624 |
| 313 | 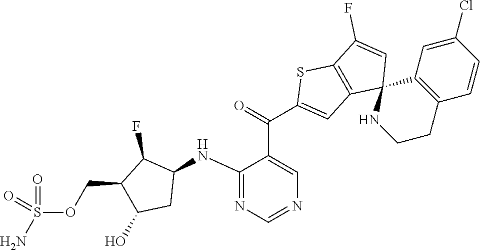 [SMILES: NS(=O)(=O)OC[C@H]1[C@@H](F)[C@@H](Nc2ncncc2C(=O)c2cc3c(s2)C(F)=C[C@@]32NCCc3ccc(Cl)cc32)C[C@@H]1O] | 624 |
| 314 | 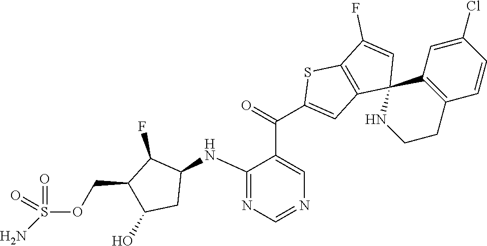 [SMILES: NS(=O)(=O)OC[C@H]1[C@@H](F)[C@@H](Nc2ncncc2C(=O)c2cc3c(s2)C(F)=C[C@]32NCCc3ccc(Cl)cc32)C[C@@H]1O] | 624 |
| 315 | 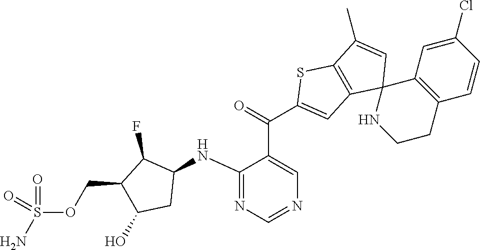 [SMILES: CC1=CC2(NCCc3ccc(Cl)cc32)c2cc(C(=O)c3cncnc3N[C@H]3C[C@H](O)[C@@H](COS(N)(=O)=O)[C@H]3F)sc21] | 620 |
| 316 | 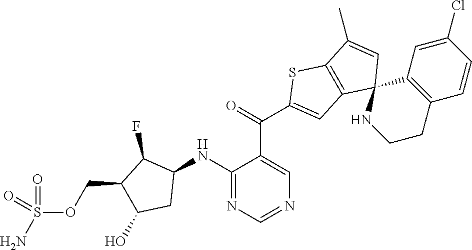 [SMILES: CC1=C[C@@]2(NCCc3ccc(Cl)cc32)c2cc(C(=O)c3cncnc3N[C@H]3C[C@H](O)[C@@H](COS(N)(=O)=O)[C@H]3F)sc21] | 620 |
| 317 | 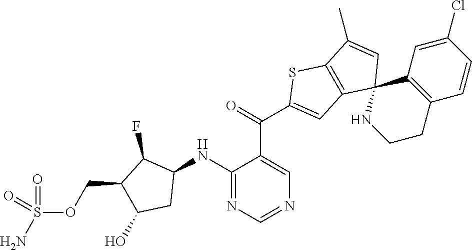 [SMILES: CC1=C[C@]2(NCCc3ccc(Cl)cc32)c2cc(C(=O)c3cncnc3N[C@H]3C[C@H](O)[C@@H](COS(N)(=O)=O)[C@H]3F)sc21] | 620 |
| 318 | 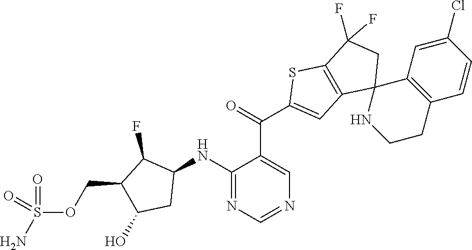 [SMILES: NS(=O)(=O)OC[C@H]1[C@@H](F)[C@@H](Nc2ncncc2C(=O)c2cc3c(s2)C(F)(F)CC32NCCc3ccc(Cl)cc32)C[C@@H]1O] | 644 |
| 319 | 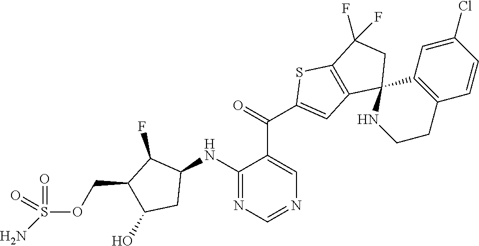 [SMILES: NS(=O)(=O)OC[C@H]1[C@@H](F)[C@@H](Nc2ncncc2C(=O)c2cc3c(s2)C(F)(F)C[C@@]32NCCc3ccc(Cl)cc32)C[C@@H]1O] | 644 |
| 320 | 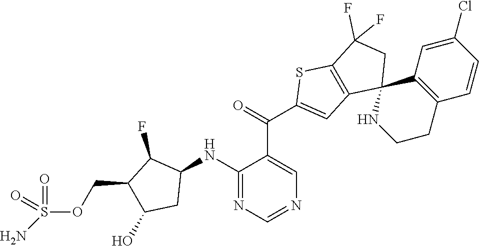 [SMILES: NS(=O)(=O)OC[C@H]1[C@@H](F)[C@@H](Nc2ncncc2C(=O)c2cc3c(s2)C(F)(F)C[C@]32NCCc3ccc(Cl)cc32)C[C@@H]1O] | 644 |
| 321 | 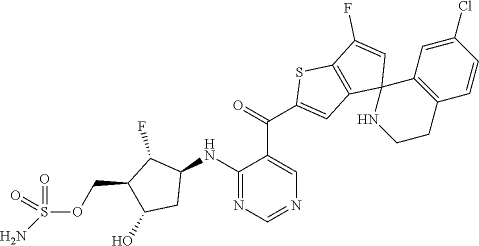 [SMILES: NS(=O)(=O)OC[C@H]1[C@H](F)[C@@H](Nc2ncncc2C(=O)c2cc3c(s2)C(F)=CC32NCCc3ccc(Cl)cc32)C[C@@H]1O] | 624 |
| 322 | 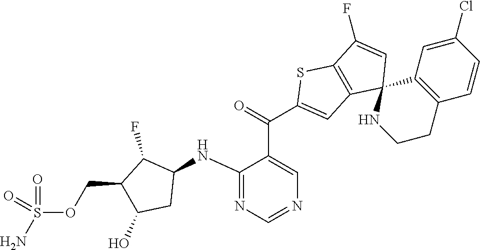 [SMILES: NS(=O)(=O)OC[C@H]1[C@H](F)[C@@H](Nc2ncncc2C(=O)c2cc3c(s2)C(F)=C[C@@]32NCCc3ccc(Cl)cc32)C[C@@H]1O] | 624 |
| 323 | 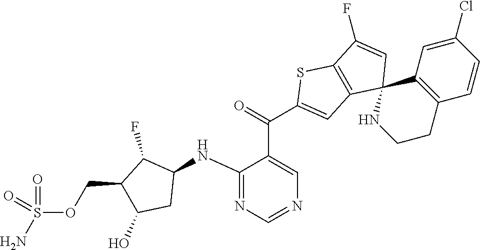 [SMILES: NS(=O)(=O)OC[C@H]1[C@H](F)[C@@H](Nc2ncncc2C(=O)c2cc3c(s2)C(F)=C[C@]32NCCc3ccc(Cl)cc32)C[C@@H]1O] | 624 |
| 324 | 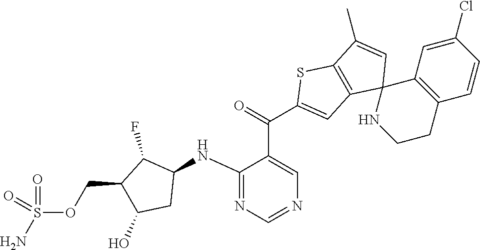 [SMILES: CC1=CC2(NCCc3ccc(Cl)cc32)c2cc(C(=O)c3cncnc3N[C@H]3C[C@H](O)[C@@H](COS(N)(=O)=O)[C@@H]3F)sc21] | 620 |
| 325 | 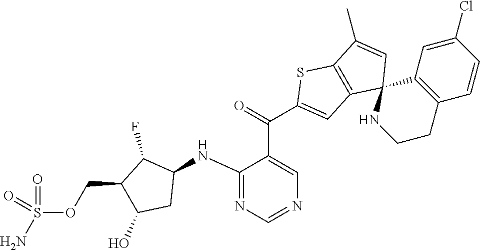 [SMILES: CC1=C[C@@]2(NCCc3ccc(Cl)cc32)c2cc(C(=O)c3cncnc3N[C@H]3C[C@H](O)[C@@H](COS(N)(=O)=O)[C@@H]3F)sc21] | 620 |
| 326 | 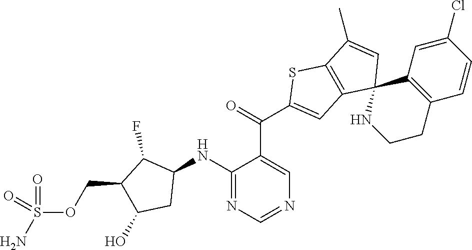 [SMILES: CC1=C[C@]2(NCCc3ccc(Cl)cc32)c2cc(C(=O)c3cncnc3N[C@H]3C[C@H](O)[C@@H](COS(N)(=O)=O)[C@@H]3F)sc21] | 620 |
| 327 | 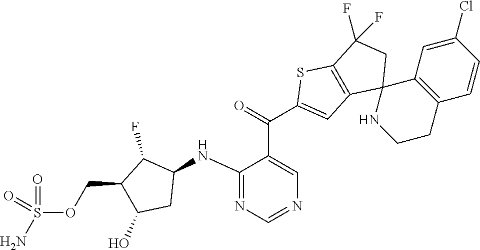 [SMILES: NS(=O)(=O)OC[C@H]1[C@H](F)[C@@H](Nc2ncncc2C(=O)c2cc3c(s2)C(F)(F)CC32NCCc3ccc(Cl)cc32)C[C@@H]1O] | 644 |
| 328 | 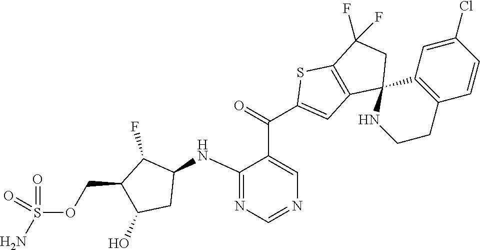 [SMILES: NS(=O)(=O)OC[C@H]1[C@H](F)[C@@H](Nc2ncncc2C(=O)c2cc3c(s2)C(F)(F)C[C@@]32NCCc3ccc(Cl)cc32)C[C@@H]1O] | 644 |
| 329 | 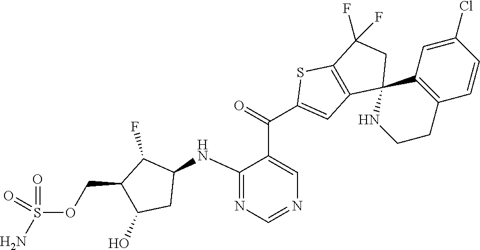 [SMILES: NS(=O)(=O)OC[C@H]1[C@H](F)[C@@H](Nc2ncncc2C(=O)c2cc3c(s2)C(F)(F)C[C@]32NCCc3ccc(Cl)cc32)C[C@@H]1O] | 644 |
| 330 | 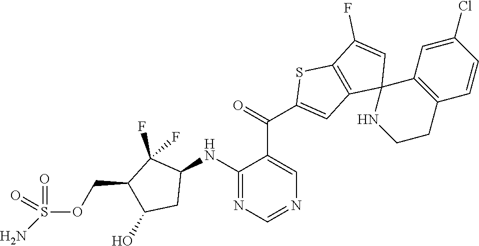 | 642 |
| 331 | 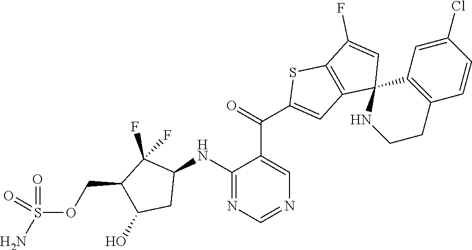 | 642 |
| 332 | 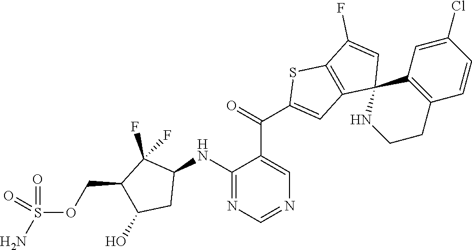 | 642 |
| 333 | 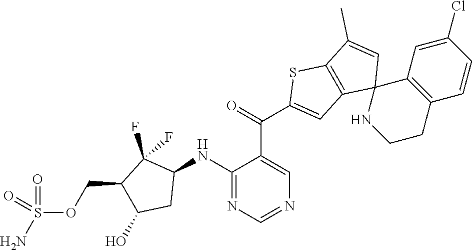 | 638 |
| 334 | 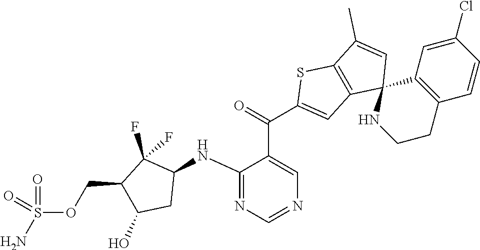 | 638 |
| 335 | 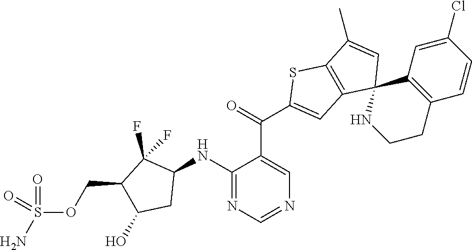 | 638 |
| 336 | 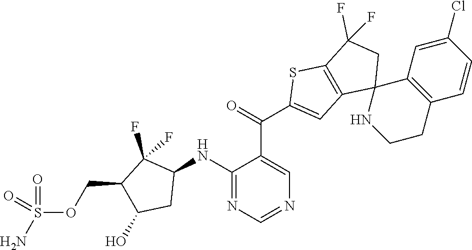 | 662 |
| 337 | 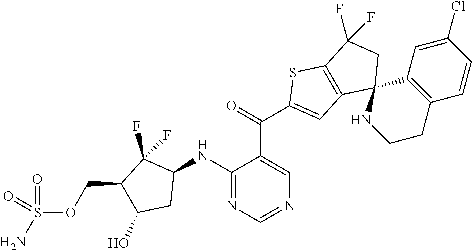 | 662 |
| 338 | 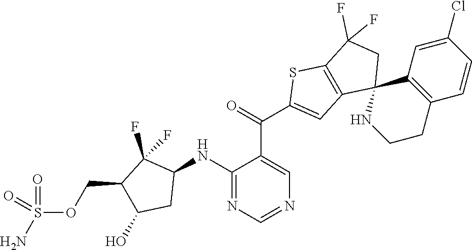 | 662 |
| 339 | 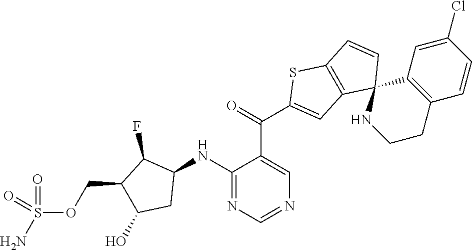 [SMILES: NS(=O)(=O)OC[C@H]1[C@@H](F)[C@@H](Nc2ncncc2C(=O)c2cc3c(s2)C=C[C@@]32NCCc3ccc(Cl)cc32)C[C@@H]1O] | 606 |
| 340 | 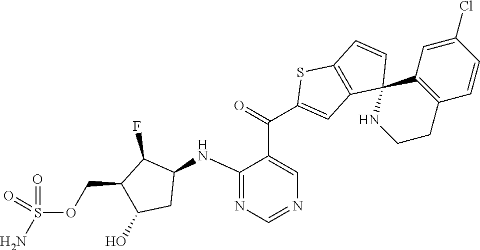 [SMILES: NS(=O)(=O)OC[C@H]1[C@@H](F)[C@@H](Nc2ncncc2C(=O)c2cc3c(s2)C=C[C@]32NCCc3ccc(Cl)cc32)C[C@@H]1O] | 606 |
| 341 | 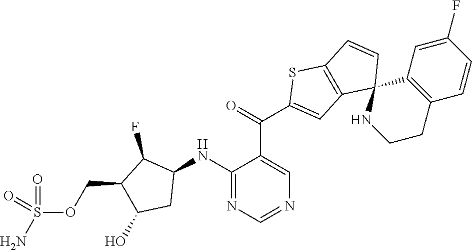 [SMILES: NS(=O)(=O)OC[C@H]1[C@@H](F)[C@@H](Nc2ncncc2C(=O)c2cc3c(s2)C=C[C@@]32NCCc3ccc(F)cc32)C[C@@H]1O] | 590 |
| 342 | 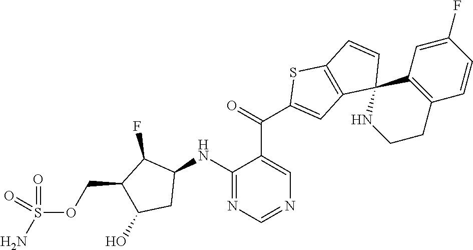 [SMILES: NS(=O)(=O)OC[C@H]1[C@@H](F)[C@@H](Nc2ncncc2C(=O)c2cc3c(s2)C=C[C@]32NCCc3ccc(F)cc32)C[C@@H]1O] | 590 |
| 343 | 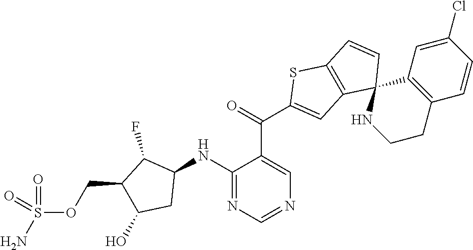 [SMILES: NS(=O)(=O)OC[C@H]1[C@H](F)[C@@H](Nc2ncncc2C(=O)c2cc3c(s2)C=C[C@@]32NCCc3ccc(Cl)cc32)C[C@@H]1O] | 606 |
| 344 | 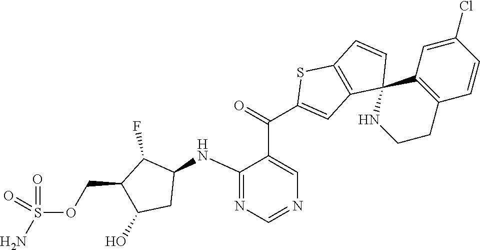 [SMILES: NS(=O)(=O)OC[C@H]1[C@H](F)[C@@H](Nc2ncncc2C(=O)c2cc3c(s2)C=C[C@]32NCCc3ccc(Cl)cc32)C[C@@H]1O] | 606 |
| 345 | 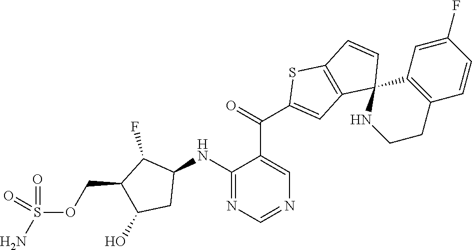 [SMILES: NS(=O)(=O)OC[C@H]1[C@H](F)[C@@H](Nc2ncncc2C(=O)c2cc3c(s2)C=C[C@@]32NCCc3ccc(F)cc32)C[C@@H]1O] | 590 |
| 346 | 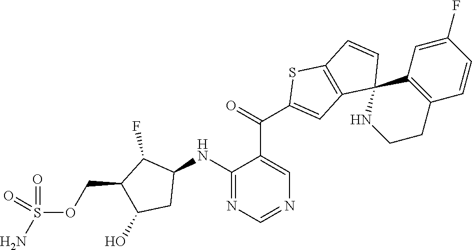 [SMILES: NS(=O)(=O)OC[C@H]1[C@H](F)[C@@H](Nc2ncncc2C(=O)c2cc3c(s2)C=C[C@]32NCCc3ccc(F)cc32)C[C@@H]1O] | 590 |
| 347 | 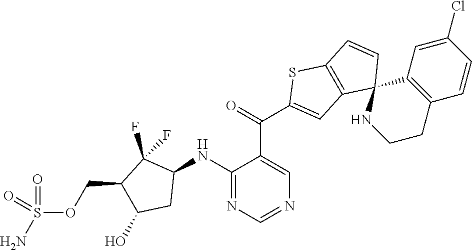 | 624 |
| 348 | 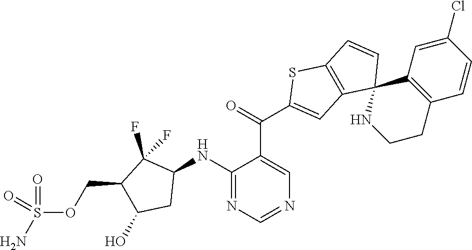 | 624 |
| 349 | 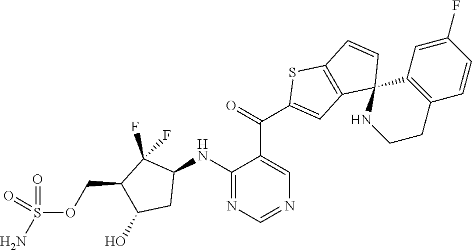 | 608 |
| 350 | 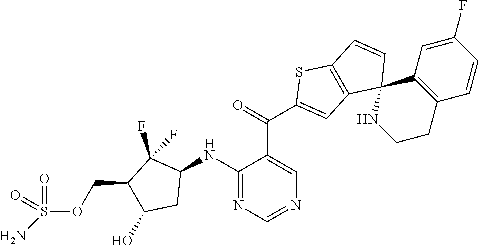 | 608 |
| 351 | 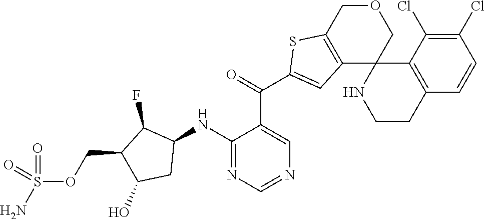 [SMILES: NS(=O)(=O)OC[C@H]1[C@@H](F)[C@@H](Nc2ncncc2C(=O)c2cc3c(s2)COCC32NCCc3ccc(Cl)c(Cl)c32)C[C@@H]1O] | 658 |
| 352 | 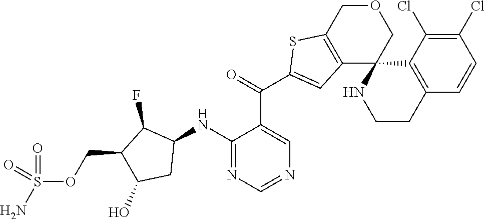 [SMILES: NS(=O)(=O)OC[C@H]1[C@@H](F)[C@@H](Nc2ncncc2C(=O)c2cc3c(s2)COC[C@@]32NCCc3ccc(Cl)c(Cl)c32)C[C@@H]1O] | 658 |
| 353 | 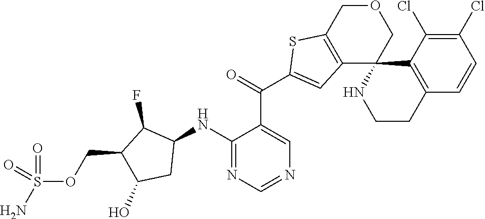 [SMILES: NS(=O)(=O)OC[C@H]1[C@@H](F)[C@@H](Nc2ncncc2C(=O)c2cc3c(s2)COC[C@]32NCCc3ccc(Cl)c(Cl)c32)C[C@@H]1O] | 658 |
| 354 | 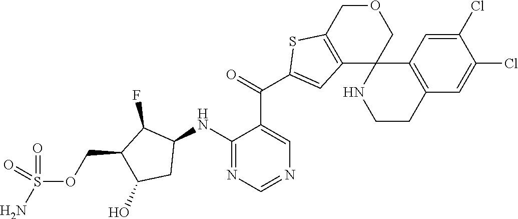 [SMILES: NS(=O)(=O)OC[C@H]1[C@@H](F)[C@@H](Nc2ncncc2C(=O)c2cc3c(s2)COCC32NCCc3cc(Cl)c(Cl)cc32)C[C@@H]1O] | 658 |
| 355 | 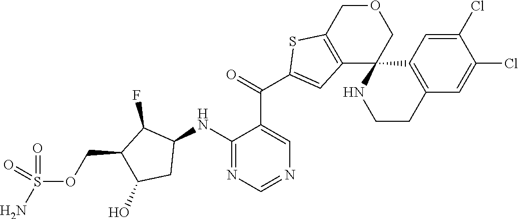 [SMILES: NS(=O)(=O)OC[C@H]1[C@@H](F)[C@@H](Nc2ncncc2C(=O)c2cc3c(s2)COC[C@@]32NCCc3cc(Cl)c(Cl)cc32)C[C@@H]1O] | 658 |
| 356 | 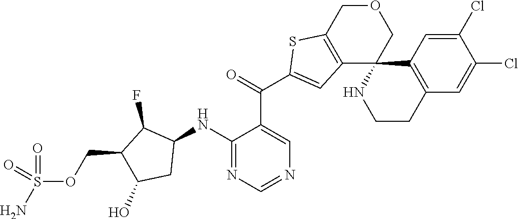 [SMILES: NS(=O)(=O)OC[C@H]1[C@@H](F)[C@@H](Nc2ncncc2C(=O)c2cc3c(s2)COC[C@]32NCCc3cc(Cl)c(Cl)cc32)C[C@@H]1O] | 658 |
| 357 | 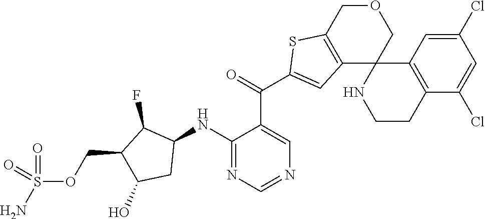 [SMILES: NS(=O)(=O)OC[C@H]1[C@@H](F)[C@@H](Nc2ncncc2C(=O)c2cc3c(s2)COCC32NCCc3c(Cl)cc(Cl)cc32)C[C@@H]1O] | 658 |
| 358 | 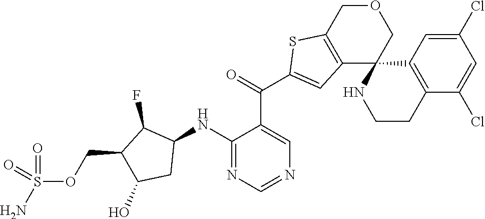 [SMILES: NS(=O)(=O)OC[C@H]1[C@@H](F)[C@@H](Nc2ncncc2C(=O)c2cc3c(s2)COC[C@@]32NCCc3c(Cl)cc(Cl)cc32)C[C@@H]1O] | 658 |
| 359 | 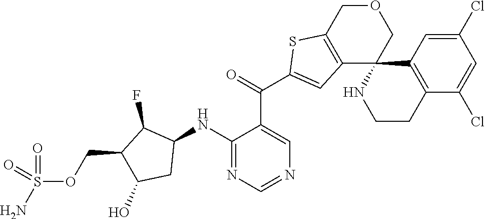 [SMILES: NS(=O)(=O)OC[C@H]1[C@@H](F)[C@@H](Nc2ncncc2C(=O)c2cc3c(s2)COC[C@]32NCCc3c(Cl)cc(Cl)cc32)C[C@@H]1O] | 658 |
| 360 | 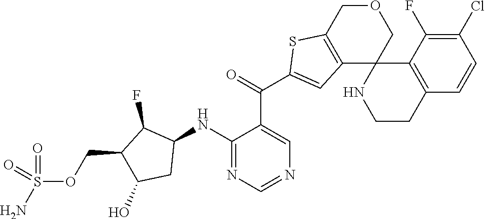 [SMILES: NS(=O)(=O)OC[C@H]1[C@@H](F)[C@@H](Nc2ncncc2C(=O)c2cc3c(s2)COCC32NCCc3ccc(Cl)c(F)c32)C[C@@H]1O] | 642 |
| 361 | 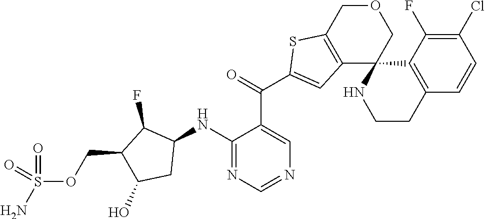 [SMILES: NS(=O)(=O)OC[C@H]1[C@@H](F)[C@@H](Nc2ncncc2C(=O)c2cc3c(s2)COC[C@@]32NCCc3ccc(Cl)c(F)c32)C[C@@H]1O] | 642 |
| 362 | 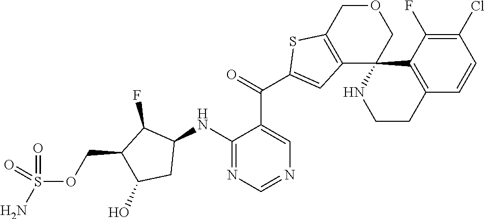 [SMILES: NS(=O)(=O)OC[C@H]1[C@@H](F)[C@@H](Nc2ncncc2C(=O)c2cc3c(s2)COC[C@]32NCCc3ccc(Cl)c(F)c32)C[C@@H]1O] | 642 |
| 363 | 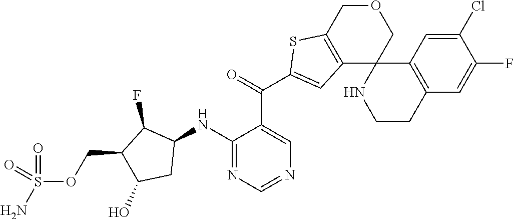 [SMILES: NS(=O)(=O)OC[C@H]1[C@@H](F)[C@@H](Nc2ncncc2C(=O)c2cc3c(s2)COCC32NCCc3cc(F)c(Cl)cc32)C[C@@H]1O] | 642 |
| 364 | 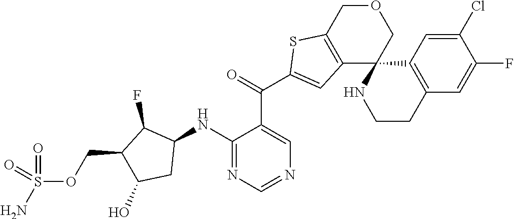 [SMILES: NS(=O)(=O)OC[C@H]1[C@@H](F)[C@@H](Nc2ncncc2C(=O)c2cc3c(s2)COC[C@@]32NCCc3cc(F)c(Cl)cc32)C[C@@H]1O] | 642 |
| 365 | 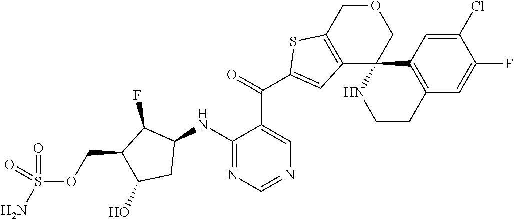 [SMILES: NS(=O)(=O)OC[C@H]1[C@@H](F)[C@@H](Nc2ncncc2C(=O)c2cc3c(s2)COC[C@]32NCCc3cc(F)c(Cl)cc32)C[C@@H]1O] | 642 |
| 366 | 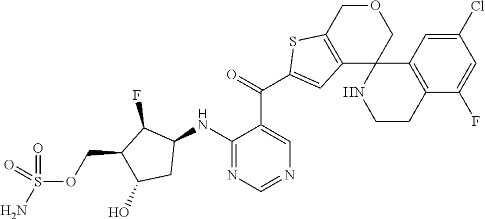 [SMILES: NS(=O)(=O)OC[C@H]1[C@@H](F)[C@@H](Nc2ncncc2C(=O)c2cc3c(s2)COCC32NCCc3c(F)cc(Cl)cc32)C[C@@H]1O] | 642 |
| 367 | 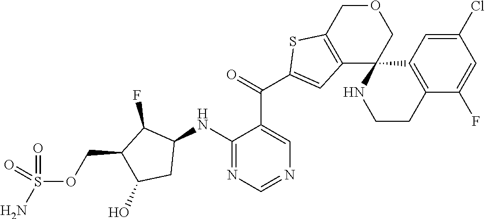 [SMILES: NS(=O)(=O)OC[C@H]1[C@@H](F)[C@@H](Nc2ncncc2C(=O)c2cc3c(s2)COC[C@@]32NCCc3c(F)cc(Cl)cc32)C[C@@H]1O] | 642 |
| 368 | 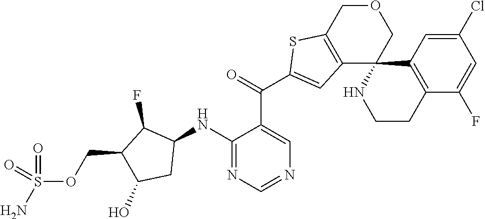 [SMILES: NS(=O)(=O)OC[C@H]1[C@@H](F)[C@@H](Nc2ncncc2C(=O)c2cc3c(s2)COC[C@]32NCCc3c(F)cc(Cl)cc32)C[C@@H]1O] | 642 |
| 369 | 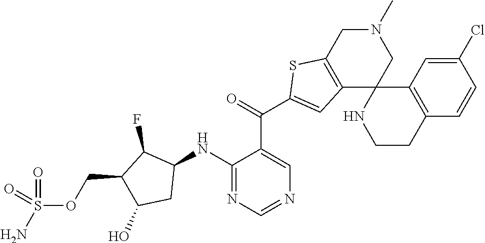 [SMILES: CN1Cc2sc(C(=O)c3cncnc3N[C@H]3C[C@H](O)[C@@H](COS(N)(=O)=O)[C@H]3F)cc2C2(C1)NCCc1ccc(Cl)cc12] | 637 |
| 370 | 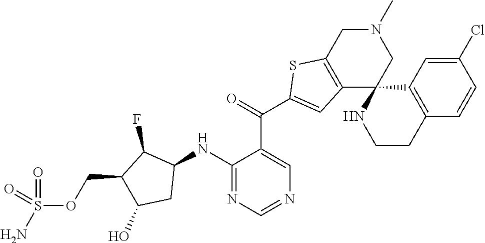 [SMILES: CN1Cc2sc(C(=O)c3cncnc3N[C@H]3C[C@H](O)[C@@H](COS(N)(=O)=O)[C@H]3F)cc2[C@@]2(C1)NCCc1ccc(Cl)cc12] | 637 |
| 371 | 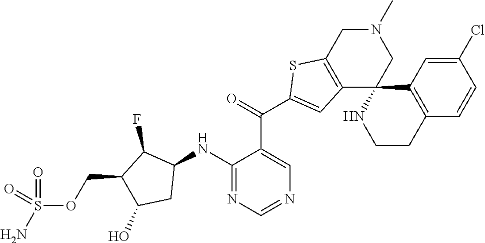 [SMILES: CN1Cc2sc(C(=O)c3cncnc3N[C@H]3C[C@H](O)[C@@H](COS(N)(=O)=O)[C@H]3F)cc2[C@]2(C1)NCCc1ccc(Cl)cc12] | 637 |
| 372 | 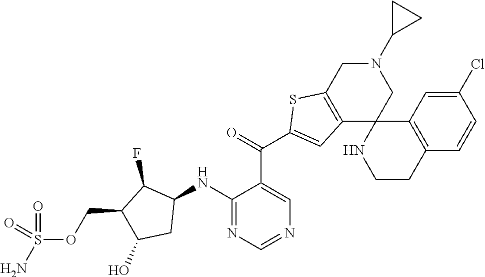 [SMILES: NS(=O)(=O)OC[C@H]1[C@@H](F)[C@@H](Nc2ncncc2C(=O)c2cc3c(s2)CN(C2CC2)CC32NCCc3ccc(Cl)cc32)C[C@@H]1O] | 663 |
| 373 | 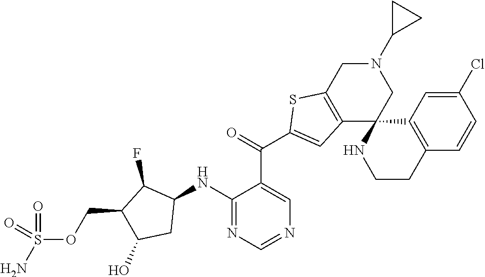 [SMILES: NS(=O)(=O)OC[C@H]1[C@@H](F)[C@@H](Nc2ncncc2C(=O)c2cc3c(s2)CN(C2CC2)C[C@@]32NCCc3ccc(Cl)cc32)C[C@@H]1O] | 663 |
| 374 | 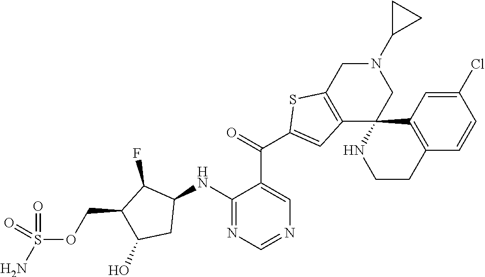 [SMILES: NS(=O)(=O)OC[C@H]1[C@@H](F)[C@@H](Nc2ncncc2C(=O)c2cc3c(s2)CN(C2CC2)C[C@]32NCCc3ccc(Cl)cc32)C[C@@H]1O] | 663 |
| 375 | 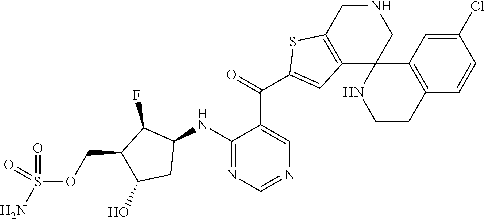 [SMILES: NS(=O)(=O)OC[C@H]1[C@@H](F)[C@@H](Nc2ncncc2C(=O)c2cc3c(s2)CNCC32NCCc3ccc(Cl)cc32)C[C@@H]1O] | 623 |
| 376 | 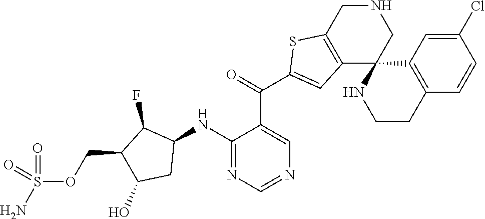 [SMILES: NS(=O)(=O)OC[C@H]1[C@@H](F)[C@@H](Nc2ncncc2C(=O)c2cc3c(s2)CNC[C@@]32NCCc3ccc(Cl)cc32)C[C@@H]1O] | 623 |
| 377 | 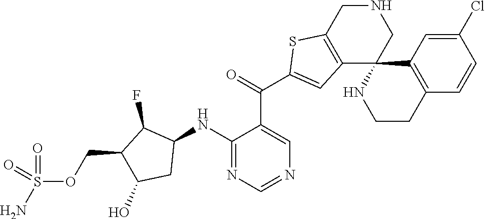 [SMILES: NS(=O)(=O)OC[C@H]1[C@@H](F)[C@@H](Nc2ncncc2C(=O)c2cc3c(s2)CNC[C@]32NCCc3ccc(Cl)cc32)C[C@@H]1O] | 623 |
| 378 | 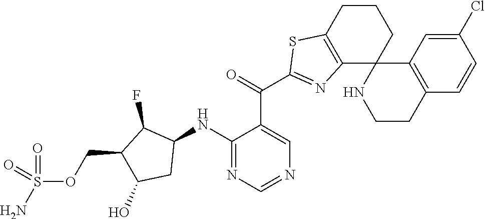 [SMILES: NS(=O)(=O)OC[C@H]1[C@@H](F)[C@@H](Nc2ncncc2C(=O)c2nc3c(s2)CCCC32NCCc3ccc(Cl)cc32)C[C@@H]1O] | 623 |
| 379 | 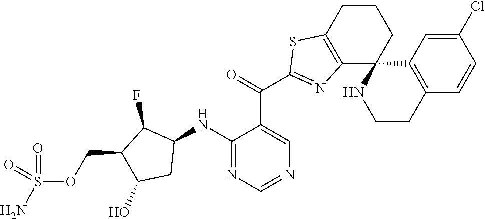 [SMILES: NS(=O)(=O)OC[C@H]1[C@@H](F)[C@@H](Nc2ncncc2C(=O)c2nc3c(s2)CCC[C@@]32NCCc3ccc(Cl)cc32)C[C@@H]1O] | 623 |
| 380 | 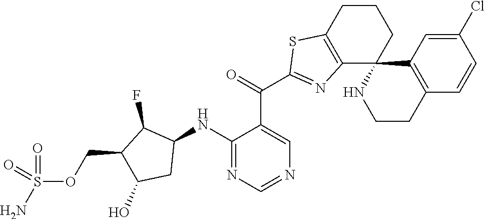 [SMILES: NS(=O)(=O)OC[C@H]1[C@@H](F)[C@@H](Nc2ncncc2C(=O)c2nc3c(s2)CCC[C@]32NCCc3ccc(Cl)cc32)C[C@@H]1O] | 623 |
| 381 | 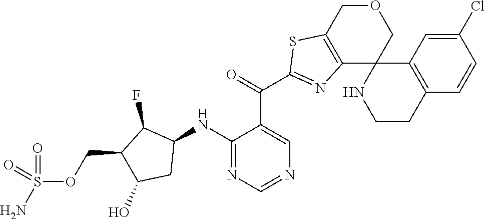 [SMILES: NS(=O)(=O)OC[C@H]1[C@@H](F)[C@@H](Nc2ncncc2C(=O)c2nc3c(s2)COCC32NCCc3ccc(Cl)cc32)C[C@@H]1O] | 625 |
| 382 | 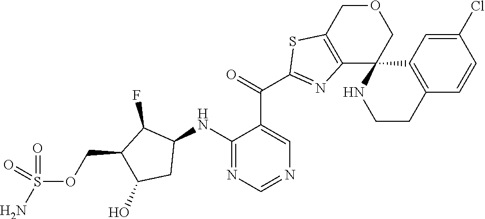 [SMILES: NS(=O)(=O)OC[C@H]1[C@@H](F)[C@@H](Nc2ncncc2C(=O)c2nc3c(s2)COC[C@@]32NCCc3ccc(Cl)cc32)C[C@@H]1O] | 625 |
| 383 | 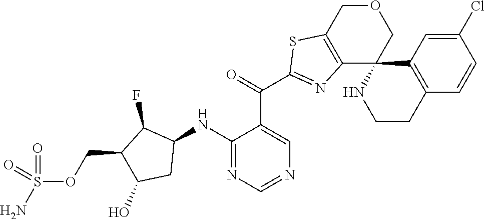 [SMILES: NS(=O)(=O)OC[C@H]1[C@@H](F)[C@@H](Nc2ncncc2C(=O)c2nc3c(s2)COC[C@]32NCCc3ccc(Cl)cc32)C[C@@H]1O] | 625 |
| 384 | 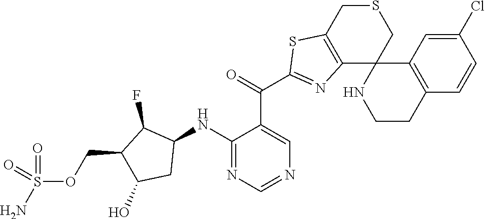 [SMILES: NS(=O)(=O)OC[C@H]1[C@@H](F)[C@@H](Nc2ncncc2C(=O)c2nc3c(s2)CSCC32NCCc3ccc(Cl)cc32)C[C@@H]1O] | 641 |
| 385 | 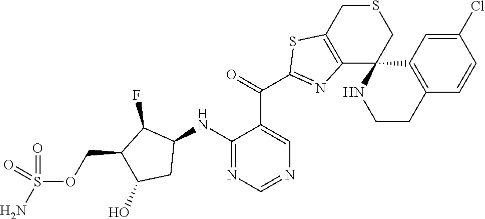 [SMILES: NS(=O)(=O)OC[C@H]1[C@@H](F)[C@@H](Nc2ncncc2C(=O)c2nc3c(s2)CSC[C@@]32NCCc3ccc(Cl)cc32)C[C@@H]1O] | 641 |
| 386 | 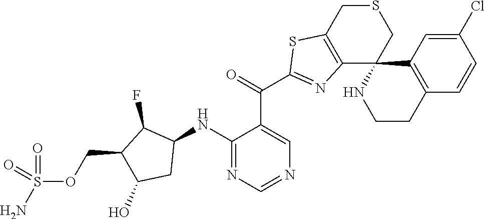 [SMILES: NS(=O)(=O)OC[C@H]1[C@@H](F)[C@@H](Nc2ncncc2C(=O)c2nc3c(s2)CSC[C@]32NCCc3ccc(Cl)cc32)C[C@@H]1O] | 641 |
| 387 | 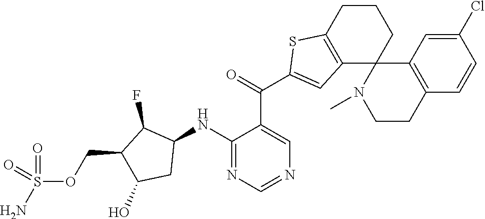 [SMILES: CN1CCc2ccc(Cl)cc2C12CCCc1sc(C(=O)c3cncnc3N[C@H]3C[C@H](O)[C@@H](COS(N)(=O)=O)[C@H]3F)cc12] | 636 |
| 388 | 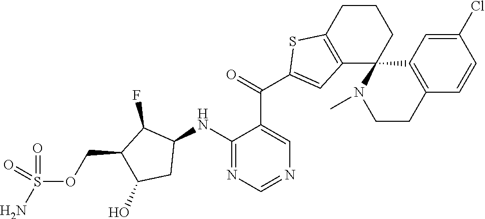 [SMILES: CN1CCc2ccc(Cl)cc2[C@]12CCCc1sc(C(=O)c3cncnc3N[C@H]3C[C@H](O)[C@@H](COS(N)(=O)=O)[C@H]3F)cc12] | 636 |
| 389 | 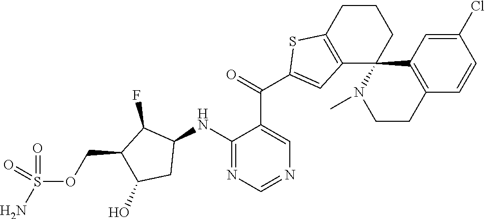 [SMILES: CN1CCc2ccc(Cl)cc2[C@@]12CCCc1sc(C(=O)c3cncnc3N[C@H]3C[C@H](O)[C@@H](COS(N)(=O)=O)[C@H]3F)cc12] | 636 |
| 390 | 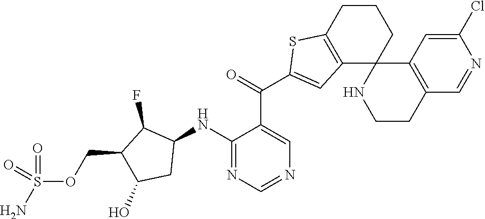 [SMILES: NS(=O)(=O)OC[C@H]1[C@@H](F)[C@@H](Nc2ncncc2C(=O)c2cc3c(s2)CCCC32NCCc3cnc(Cl)cc32)C[C@@H]1O] | 623 |
| 391 | 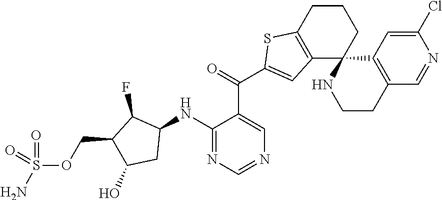 [SMILES: NS(=O)(=O)OC[C@H]1[C@@H](F)[C@@H](Nc2ncncc2C(=O)c2cc3c(s2)CCC[C@@]32NCCc3cnc(Cl)cc32)C[C@@H]1O] | 623 |
| 392 | 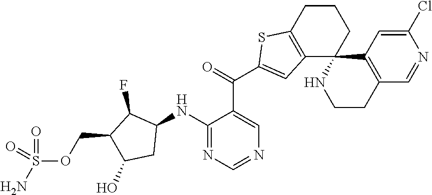 [SMILES: NS(=O)(=O)OC[C@H]1[C@@H](F)[C@@H](Nc2ncncc2C(=O)c2cc3c(s2)CCC[C@]32NCCc3cnc(Cl)cc32)C[C@@H]1O] | 623 |
| 393 | 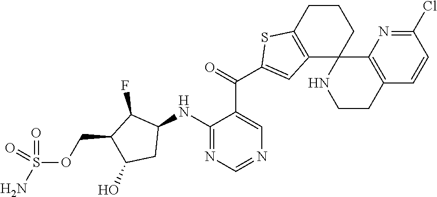 [SMILES: NS(=O)(=O)OC[C@H]1[C@@H](F)[C@@H](Nc2ncncc2C(=O)c2cc3c(s2)CCCC32NCCc3ccc(Cl)nc32)C[C@@H]1O] | 623 |
| 394 | 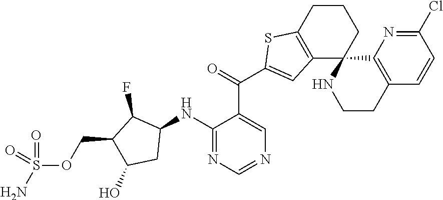 [SMILES: NS(=O)(=O)OC[C@H]1[C@@H](F)[C@@H](Nc2ncncc2C(=O)c2cc3c(s2)CCC[C@@]32NCCc3ccc(Cl)nc32)C[C@@H]1O] | 623 |
| 395 | 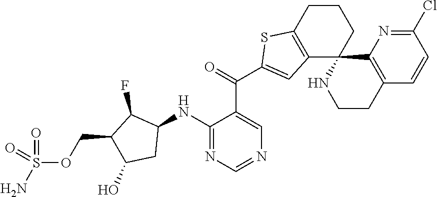 [SMILES: NS(=O)(=O)OC[C@H]1[C@@H](F)[C@@H](Nc2ncncc2C(=O)c2cc3c(s2)CCC[C@]32NCCc3ccc(Cl)nc32)C[C@@H]1O] | 623 |
| 396 | 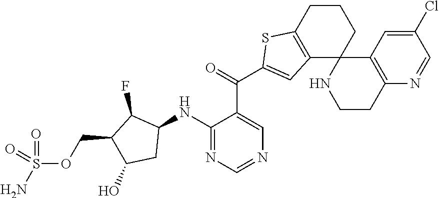 [SMILES: NS(=O)(=O)OC[C@H]1[C@@H](F)[C@@H](Nc2ncncc2C(=O)c2cc3c(s2)CCCC32NCCc3ncc(Cl)cc32)C[C@@H]1O] | 623 |
| 397 | 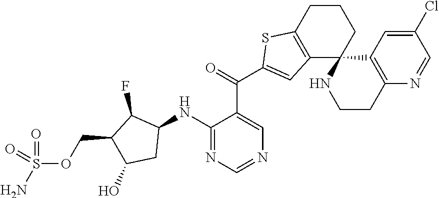 [SMILES: NS(=O)(=O)OC[C@H]1[C@@H](F)[C@@H](Nc2ncncc2C(=O)c2cc3c(s2)CCC[C@@]32NCCc3ncc(Cl)cc32)C[C@@H]1O] | 623 |
| 398 | 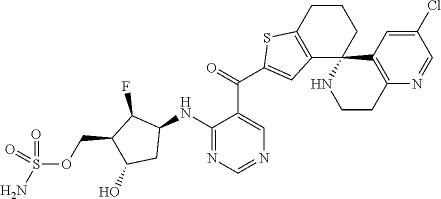 [SMILES: NS(=O)(=O)OC[C@H]1[C@@H](F)[C@@H](Nc2ncncc2C(=O)c2cc3c(s2)CCC[C@]32NCCc3ncc(Cl)cc32)C[C@@H]1O] | 623 |
| 399 | 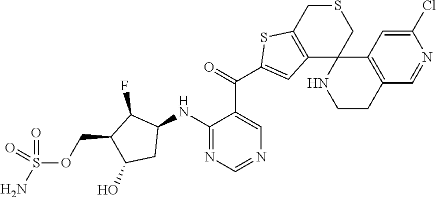 [SMILES: NS(=O)(=O)OC[C@H]1[C@@H](F)[C@@H](Nc2ncncc2C(=O)c2cc3c(s2)CSCC32NCCc3cnc(Cl)cc32)C[C@@H]1O] | 641 |
| 400 | 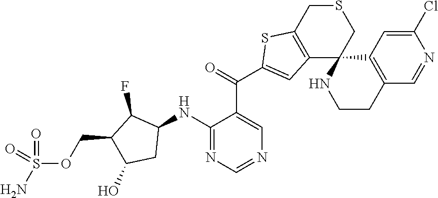 [SMILES: NS(=O)(=O)OC[C@H]1[C@@H](F)[C@@H](Nc2ncncc2C(=O)c2cc3c(s2)CSC[C@@]32NCCc3cnc(Cl)cc32)C[C@@H]1O] | 641 |
| 401 | 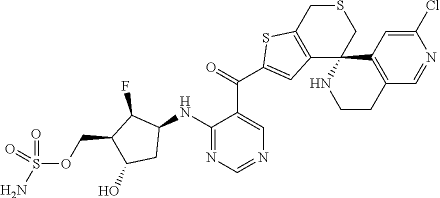 [SMILES: NS(=O)(=O)OC[C@H]1[C@@H](F)[C@@H](Nc2ncncc2C(=O)c2cc3c(s2)CSC[C@]32NCCc3cnc(Cl)cc32)C[C@@H]1O] | 641 |
| 402 | 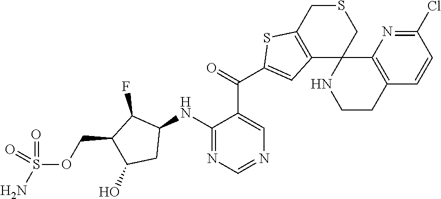 [SMILES: NS(=O)(=O)OC[C@H]1[C@@H](F)[C@@H](Nc2ncncc2C(=O)c2cc3c(s2)CSCC32NCCc3ccc(Cl)nc32)C[C@@H]1O] | 641 |
| 403 | 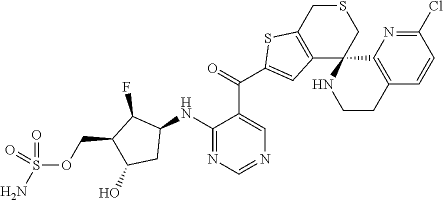 [SMILES: NS(=O)(=O)OC[C@H]1[C@@H](F)[C@@H](Nc2ncncc2C(=O)c2cc3c(s2)CSC[C@@]32NCCc3ccc(Cl)nc32)C[C@@H]1O] | 641 |
| 404 | 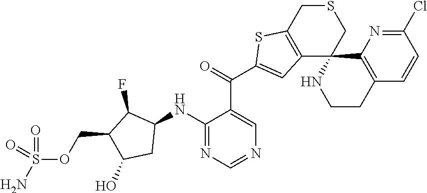 [SMILES: NS(=O)(=O)OC[C@H]1[C@@H](F)[C@@H](Nc2ncncc2C(=O)c2cc3c(s2)CSC[C@]32NCCc3ccc(Cl)nc32)C[C@@H]1O] | 641 |
| 405 | 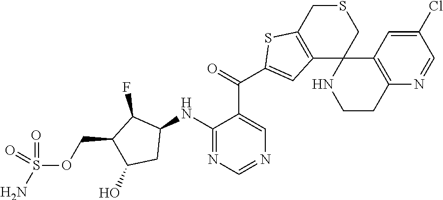 [SMILES: NS(=O)(=O)OC[C@H]1[C@@H](F)[C@@H](Nc2ncncc2C(=O)c2cc3c(s2)CSCC32NCCc3ncc(Cl)cc32)C[C@@H]1O] | 641 |
| 406 | 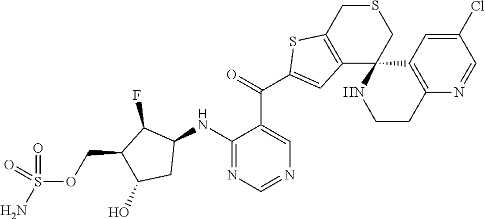 [SMILES: NS(=O)(=O)OC[C@H]1[C@@H](F)[C@@H](Nc2ncncc2C(=O)c2cc3c(s2)CSC[C@@]32NCCc3ncc(Cl)cc32)C[C@@H]1O] | 641 |
| 407 | 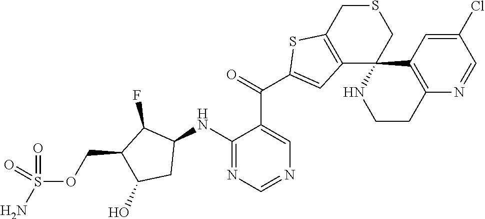 [SMILES: NS(=O)(=O)OC[C@H]1[C@@H](F)[C@@H](Nc2ncncc2C(=O)c2cc3c(s2)CSC[C@]32NCCc3ncc(Cl)cc32)C[C@@H]1O] | 641 |
| 408 | 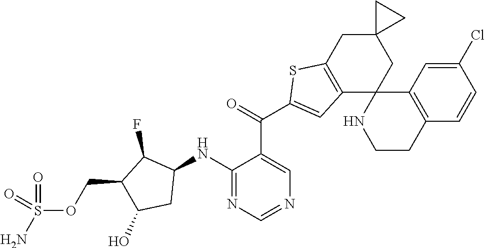 [SMILES: NS(=O)(=O)OC[C@H]1[C@@H](F)[C@@H](Nc2ncncc2C(=O)c2cc3c(s2)CC2(CC2)CC32NCCc3ccc(Cl)cc32)C[C@@H]1O] | 648 |
| 409 | 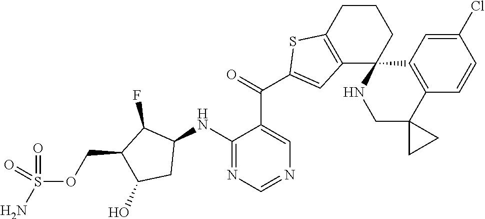 [SMILES: NS(=O)(=O)OC[C@H]1[C@@H](F)[C@@H](Nc2ncncc2C(=O)c2cc3c(s2)CCC[C@@]32NCC3(CC3)c3ccc(Cl)cc32)C[C@@H]1O] | 648 |
| 410 | 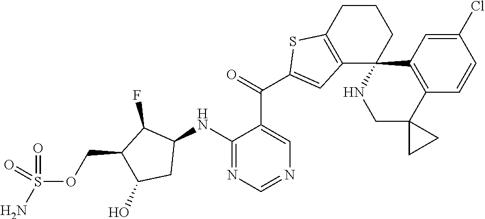 [SMILES: NS(=O)(=O)OC[C@H]1[C@@H](F)[C@@H](Nc2ncncc2C(=O)c2cc3c(s2)CCC[C@]32NCC3(CC3)c3ccc(Cl)cc32)C[C@@H]1O] | 648 |
| 411 | 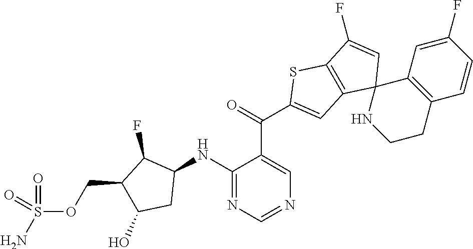 [SMILES: NS(=O)(=O)OC[C@H]1[C@@H](F)[C@@H](Nc2ncncc2C(=O)c2cc3c(s2)C(F)=CC32NCCc3ccc(F)cc32)C[C@@H]1O] | 608 |
| 412 | 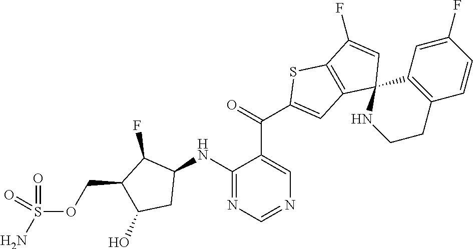 [SMILES: NS(=O)(=O)OC[C@H]1[C@@H](F)[C@@H](Nc2ncncc2C(=O)c2cc3c(s2)C(F)=C[C@@]32NCCc3ccc(F)cc32)C[C@@H]1O] | 608 |
| 413 | 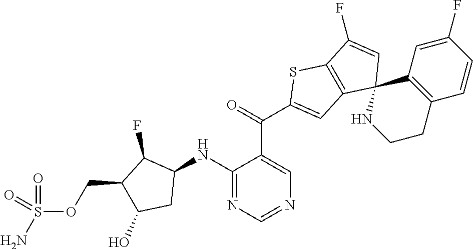 [SMILES: NS(=O)(=O)OC[C@H]1[C@@H](F)[C@@H](Nc2ncncc2C(=O)c2cc3c(s2)C(F)=C[C@]32NCCc3ccc(F)cc32)C[C@@H]1O] | 608 |
| 414 | 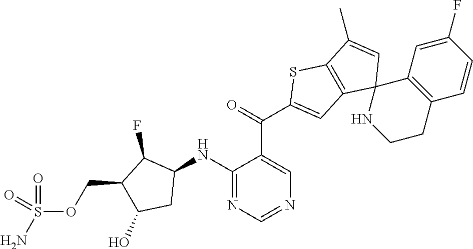 [SMILES: CC1=CC2(NCCc3ccc(F)cc32)c2cc(C(=O)c3cncnc3N[C@H]3C[C@H](O)[C@@H](COS(N)(=O)=O)[C@H]3F)sc21] | 604 |
| 415 | 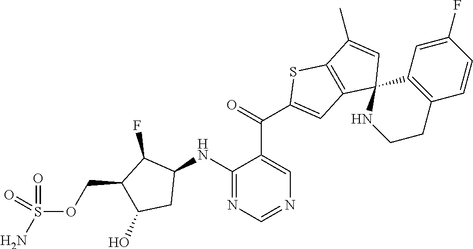 [SMILES: CC1=C[C@@]2(NCCc3ccc(F)cc32)c2cc(C(=O)c3cncnc3N[C@H]3C[C@H](O)[C@@H](COS(N)(=O)=O)[C@H]3F)sc21] | 604 |
| 416 | 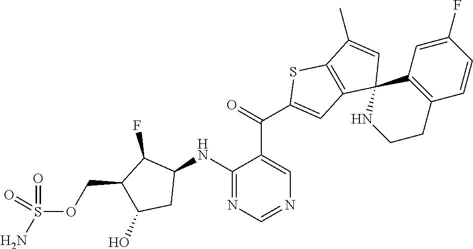 [SMILES: CC1=C[C@]2(NCCc3ccc(F)cc32)c2cc(C(=O)c3cncnc3N[C@H]3C[C@H](O)[C@@H](COS(N)(=O)=O)[C@H]3F)sc21] | 604 |
| 417 | 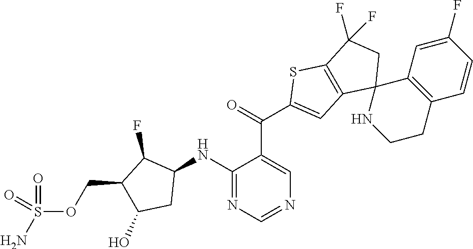 [SMILES: NS(=O)(=O)OC[C@H]1[C@@H](F)[C@@H](Nc2ncncc2C(=O)c2cc3c(s2)C(F)(F)CC32NCCc3ccc(F)cc32)C[C@@H]1O] | 628 |
| 418 | 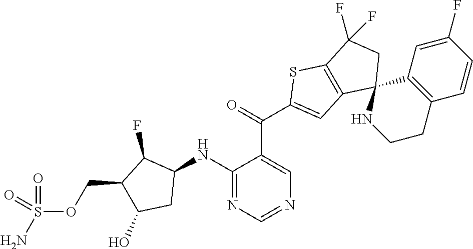 [SMILES: NS(=O)(=O)OC[C@H]1[C@@H](F)[C@@H](Nc2ncncc2C(=O)c2cc3c(s2)C(F)(F)C[C@@]32NCCc3ccc(F)cc32)C[C@@H]1O] | 628 |
| 419 | 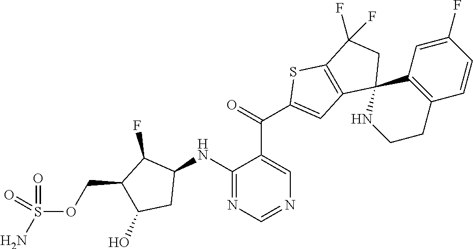 [SMILES: NS(=O)(=O)OC[C@H]1[C@@H](F)[C@@H](Nc2ncncc2C(=O)c2cc3c(s2)C(F)(F)C[C@]32NCCc3ccc(F)cc32)C[C@@H]1O] | 628 |
| 420 | 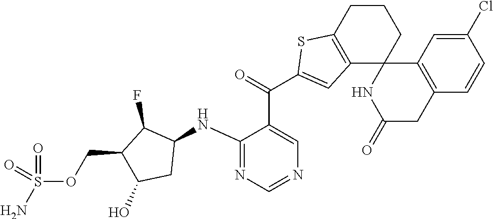 [SMILES: NS(=O)(=O)OC[C@H]1[C@@H](F)[C@@H](Nc2ncncc2C(=O)c2cc3c(s2)CCCC32NC(=O)Cc3ccc(Cl)cc32)C[C@@H]1O] | 636 |
| 421 | 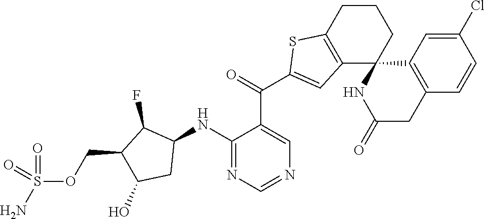 [SMILES: NS(=O)(=O)OC[C@H]1[C@@H](F)[C@@H](Nc2ncncc2C(=O)c2cc3c(s2)CCC[C@@]32NC(=O)Cc3ccc(Cl)cc32)C[C@@H]1O] | 636 |
| 422 | 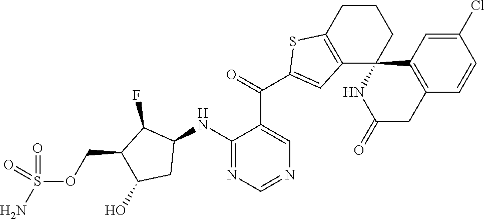 [SMILES: NS(=O)(=O)OC[C@H]1[C@@H](F)[C@@H](Nc2ncncc2C(=O)c2cc3c(s2)CCC[C@]32NC(=O)Cc3ccc(Cl)cc32)C[C@@H]1O] | 636 |
| 423 | 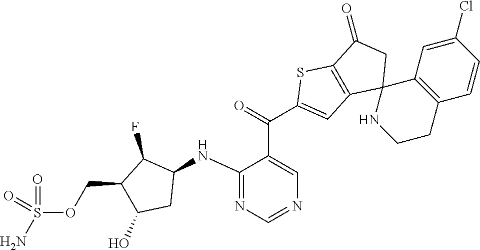 [SMILES: NS(=O)(=O)OC[C@H]1[C@@H](F)[C@@H](Nc2ncncc2C(=O)c2cc3c(s2)C(=O)CC32NCCc3ccc(Cl)cc32)C[C@@H]1O] | 622 |
| 424 | 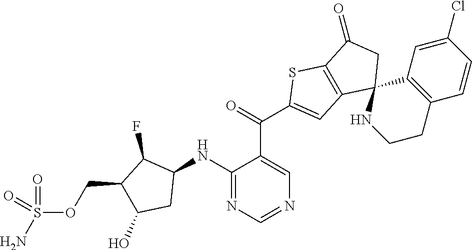 [SMILES: NS(=O)(=O)OC[C@H]1[C@@H](F)[C@@H](Nc2ncncc2C(=O)c2cc3c(s2)C(=O)C[C@@]32NCCc3ccc(Cl)cc32)C[C@@H]1O] | 622 |
| 425 | 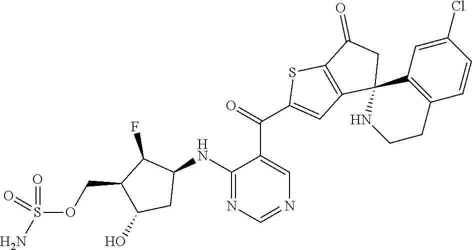 [SMILES: NS(=O)(=O)OC[C@H]1[C@@H](F)[C@@H](Nc2ncncc2C(=O)c2cc3c(s2)C(=O)C[C@]32NCCc3ccc(Cl)cc32)C[C@@H]1O] | 622 |
| 426 | 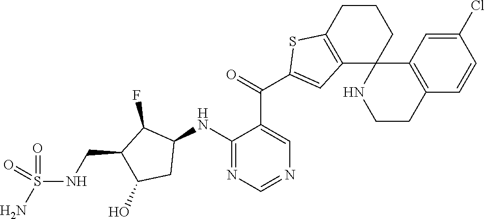 [SMILES: NS(=O)(=O)NC[C@H]1[C@@H](F)[C@@H](Nc2ncncc2C(=O)c2cc3c(s2)CCCC32NCCc3ccc(Cl)cc32)C[C@@H]1O] | 621 |
| 427 | 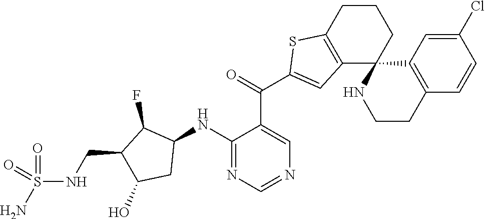 [SMILES: NS(=O)(=O)NC[C@H]1[C@@H](F)[C@@H](Nc2ncncc2C(=O)c2cc3c(s2)CCC[C@@]32NCCc3ccc(Cl)cc32)C[C@@H]1O] | 621 |
| 428 | 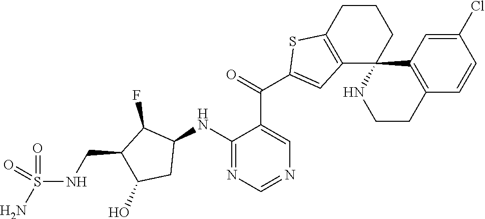 [SMILES: NS(=O)(=O)NC[C@H]1[C@@H](F)[C@@H](Nc2ncncc2C(=O)c2cc3c(s2)CCC[C@]32NCCc3ccc(Cl)cc32)C[C@@H]1O] | 621 |
| 429 | 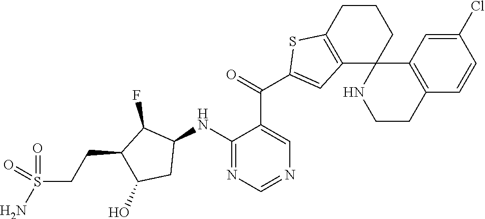 [SMILES: NS(=O)(=O)CC[C@H]1[C@@H](F)[C@@H](Nc2ncncc2C(=O)c2cc3c(s2)CCCC32NCCc3ccc(Cl)cc32)C[C@@H]1O] | 620 |
| 430 | 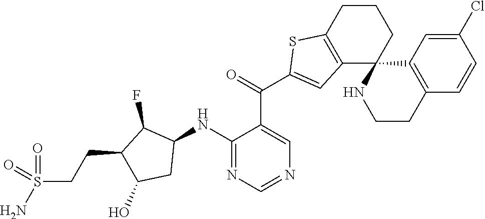 [SMILES: NS(=O)(=O)CC[C@H]1[C@@H](F)[C@@H](Nc2ncncc2C(=O)c2cc3c(s2)CCC[C@@]32NCCc3ccc(Cl)cc32)C[C@@H]1O] | 620 |
| 431 | 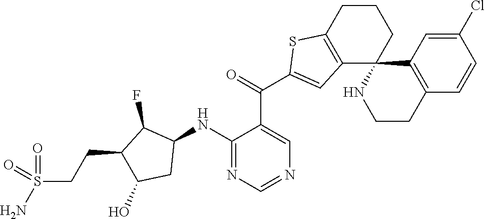 [SMILES: NS(=O)(=O)CC[C@H]1[C@@H](F)[C@@H](Nc2ncncc2C(=O)c2cc3c(s2)CCC[C@]32NCCc3ccc(Cl)cc32)C[C@@H]1O] | 620 |
| 432 | 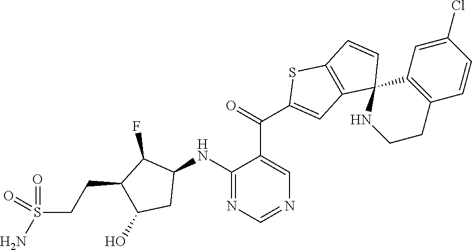 [SMILES: NS(=O)(=O)CC[C@H]1[C@@H](F)[C@@H](Nc2ncncc2C(=O)c2cc3c(s2)C=C[C@@]32NCCc3ccc(Cl)cc32)C[C@@H]1O] | 604 |
| 433 | 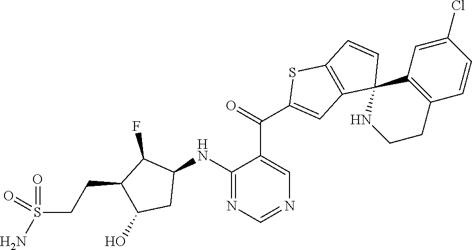 [SMILES: NS(=O)(=O)CC[C@H]1[C@@H](F)[C@@H](Nc2ncncc2C(=O)c2cc3c(s2)C=C[C@]32NCCc3ccc(Cl)cc32)C[C@@H]1O] | 604 |
| 434 | 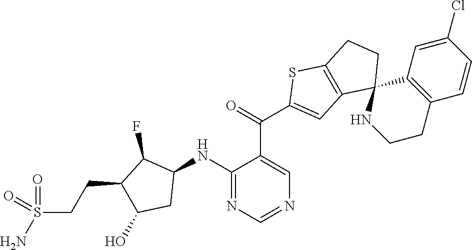 [SMILES: NS(=O)(=O)CC[C@H]1[C@@H](F)[C@@H](Nc2ncncc2C(=O)c2cc3c(s2)CC[C@@]32NCCc3ccc(Cl)cc32)C[C@@H]1O] | 606 |
| 435 | 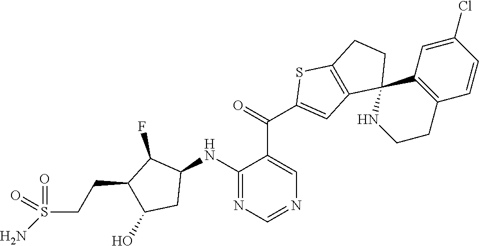 [SMILES: NS(=O)(=O)CC[C@H]1[C@@H](F)[C@@H](Nc2ncncc2C(=O)c2cc3c(s2)CC[C@]32NCCc3ccc(Cl)cc32)C[C@@H]1O] | 606 |
| 436 | 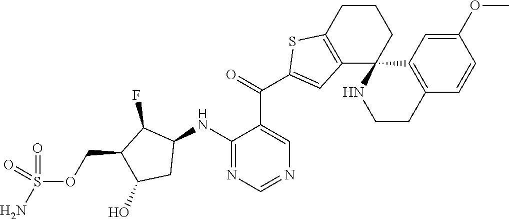 [SMILES: COc1ccc2c(c1)[C@]1(CCCc3sc(C(=O)c4cncnc4N[C@H]4C[C@H](O)[C@@H](COS(N)(=O)=O)[C@H]4F)cc31)NCC2] | 618 |
| 437 | 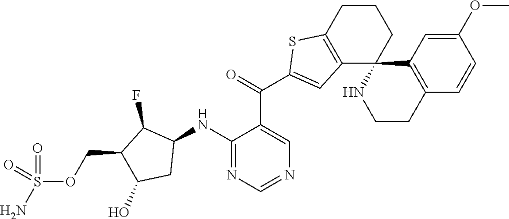 [SMILES: COc1ccc2c(c1)[C@@]1(CCCc3sc(C(=O)c4cncnc4N[C@H]4C[C@H](O)[C@@H](COS(N)(=O)=O)[C@H]4F)cc31)NCC2] | 618 |
| 438 | 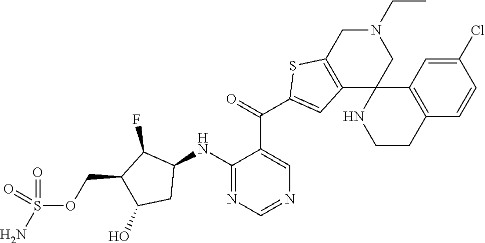 [SMILES: CCN1Cc2sc(C(=O)c3cncnc3N[C@H]3C[C@H](O)[C@@H](COS(N)(=O)=O)[C@H]3F)cc2C2(C1)NCCc1ccc(Cl)cc12] | 651 |
| 439 | 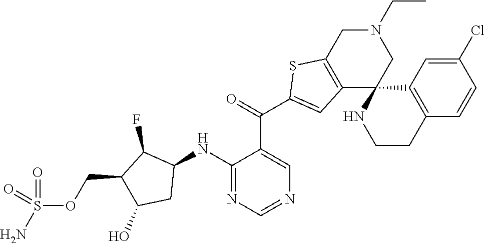 [SMILES: CCN1Cc2sc(C(=O)c3cncnc3N[C@H]3C[C@H](O)[C@@H](COS(N)(=O)=O)[C@H]3F)cc2[C@@]2(C1)NCCc1ccc(Cl)cc12] | 651 |
| 440 | 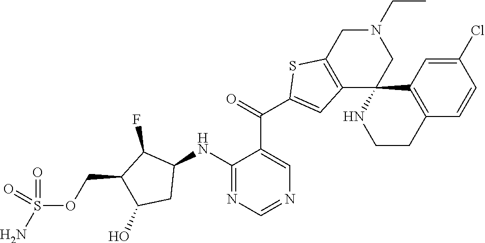 [SMILES: CCN1Cc2sc(C(=O)c3cncnc3N[C@H]3C[C@H](O)[C@@H](COS(N)(=O)=O)[C@H]3F)cc2[C@]2(C1)NCCc1ccc(Cl)cc12] | 651 |
| 441 | 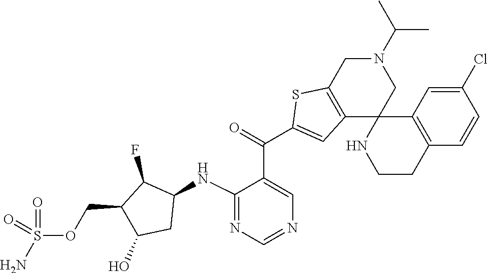 [SMILES: CC(C)N1Cc2sc(C(=O)c3cncnc3N[C@H]3C[C@H](O)[C@@H](COS(N)(=O)=O)[C@H]3F)cc2C2(C1)NCCc1ccc(Cl)cc12] | 665 |
| 442 | 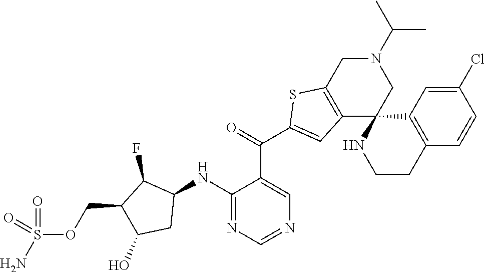 [SMILES: CC(C)N1Cc2sc(C(=O)c3cncnc3N[C@H]3C[C@H](O)[C@@H](COS(N)(=O)=O)[C@H]3F)cc2[C@@]2(C1)NCCc1ccc(Cl)cc12] | 665 |
| 443 | 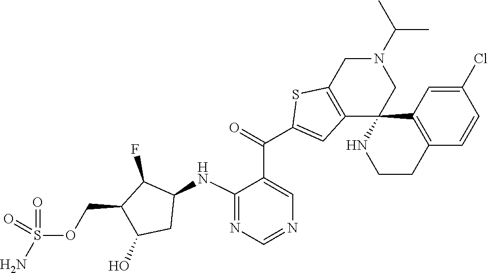 [SMILES: CC(C)N1Cc2sc(C(=O)c3cncnc3N[C@H]3C[C@H](O)[C@@H](COS(N)(=O)=O)[C@H]3F)cc2[C@]2(C1)NCCc1ccc(Cl)cc12] | 665 |
| 444 | 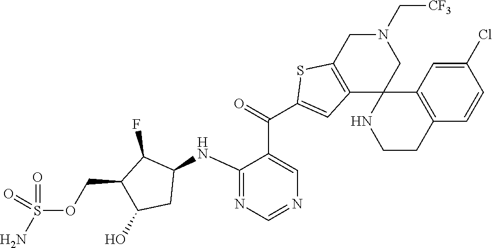 [SMILES: NS(=O)(=O)OC[C@H]1[C@@H](F)[C@@H](Nc2ncncc2C(=O)c2cc3c(s2)CN(CC(F)(F)F)CC32NCCc3ccc(Cl)cc32)C[C@@H]1O] | 705 |
| 445 | 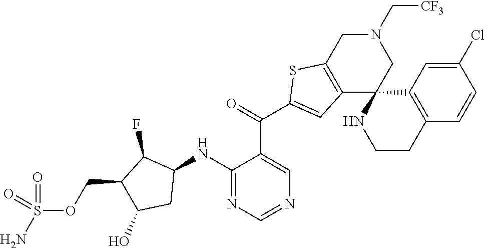 [SMILES: NS(=O)(=O)OC[C@H]1[C@@H](F)[C@@H](Nc2ncncc2C(=O)c2cc3c(s2)CN(CC(F)(F)F)C[C@@]32NCCc3ccc(Cl)cc32)C[C@@H]1O] | 705 |
| 446 | 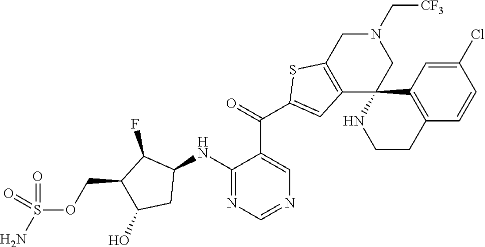 [SMILES: NS(=O)(=O)OC[C@H]1[C@@H](F)[C@@H](Nc2ncncc2C(=O)c2cc3c(s2)CN(CC(F)(F)F)C[C@]32NCCc3ccc(Cl)cc32)C[C@@H]1O] | 705 |
| 447 | 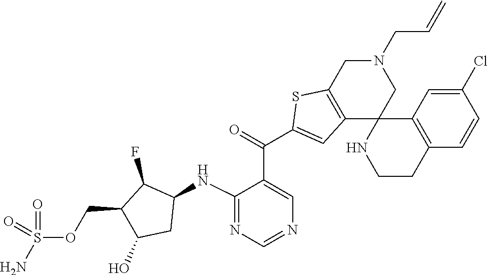 [SMILES: C=CCN1Cc2sc(C(=O)c3cncnc3N[C@H]3C[C@H](O)[C@@H](COS(N)(=O)=O)[C@H]3F)cc2C2(C1)NCCc1ccc(Cl)cc12] | 663 |
| 448 | 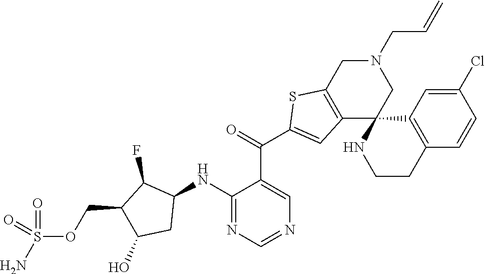 [SMILES: C=CCN1Cc2sc(C(=O)c3cncnc3N[C@H]3C[C@H](O)[C@@H](COS(N)(=O)=O)[C@H]3F)cc2[C@@]2(C1)NCCc1ccc(Cl)cc12] | 663 |
| 449 | 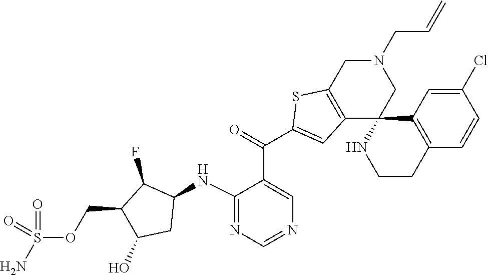 [SMILES: C=CCN1Cc2sc(C(=O)c3cncnc3N[C@H]3C[C@H](O)[C@@H](COS(N)(=O)=O)[C@H]3F)cc2[C@]2(C1)NCCc1ccc(Cl)cc12] | 663 |
| 450 | 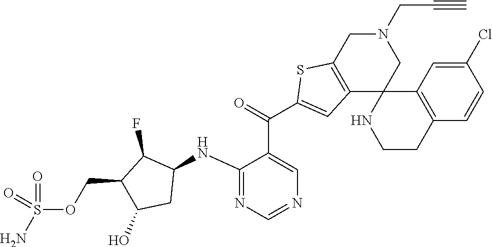 [SMILES: C#CCN1Cc2sc(C(=O)c3cncnc3N[C@H]3C[C@H](O)[C@@H](COS(N)(=O)=O)[C@H]3F)cc2C2(C1)NCCc1ccc(Cl)cc12] | 661 |
| 451 | 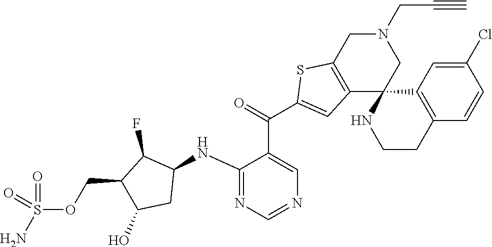 [SMILES: C#CCN1Cc2sc(C(=O)c3cncnc3N[C@H]3C[C@H](O)[C@@H](COS(N)(=O)=O)[C@H]3F)cc2[C@@]2(C1)NCCc1ccc(Cl)cc12] | 661 |
| 452 | 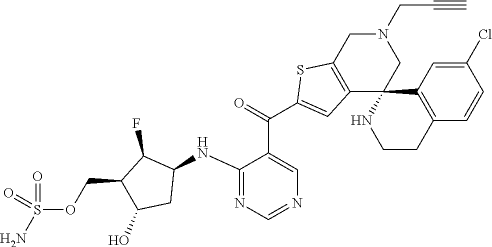 [SMILES: C#CCN1Cc2sc(C(=O)c3cncnc3N[C@H]3C[C@H](O)[C@@H](COS(N)(=O)=O)[C@H]3F)cc2[C@]2(C1)NCCc1ccc(Cl)cc12] | 661 |
| 453 | 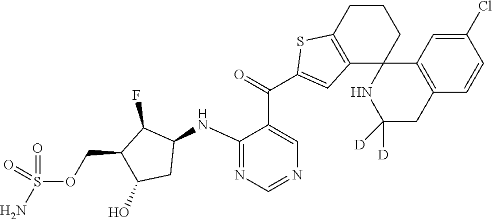 [SMILES: [2H]C1([2H])Cc2ccc(Cl)cc2C2(CCCc3sc(C(=O)c4cncnc4N[C@H]4C[C@H](O)[C@@H](COS(N)(=O)=O)[C@H]4F)cc32)N1] | 624 |
| 454 | 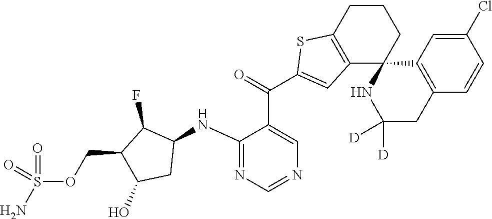 [SMILES: [2H]C1([2H])Cc2ccc(Cl)cc2[C@]2(CCCc3sc(C(=O)c4cncnc4N[C@H]4C[C@H](O)[C@@H](COS(N)(=O)=O)[C@H]4F)cc32)N1] | 624 |
| 455 | 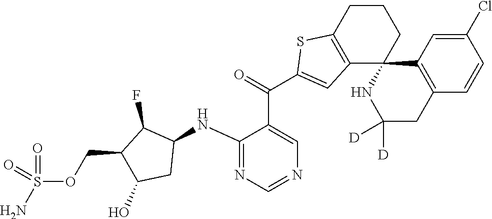 [SMILES: [2H]C1([2H])Cc2ccc(Cl)cc2[C@@]2(CCCc3sc(C(=O)c4cncnc4N[C@H]4C[C@H](O)[C@@H](COS(N)(=O)=O)[C@H]4F)cc32)N1] | 624 |
| 456 | 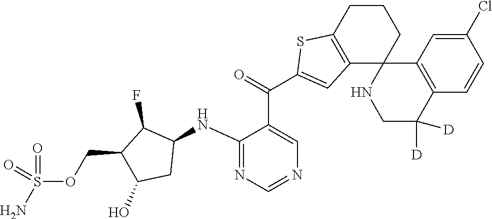 [SMILES: [2H]C1([2H])CNC2(CCCc3sc(C(=O)c4cncnc4N[C@H]4C[C@H](O)[C@@H](COS(N)(=O)=O)[C@H]4F)cc32)c2cc(Cl)ccc21] | 624 |
| 457 | 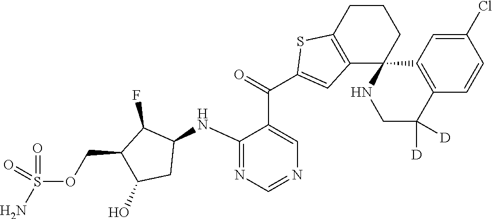 [SMILES: [2H]C1([2H])CN[C@@]2(CCCc3sc(C(=O)c4cncnc4N[C@H]4C[C@H](O)[C@@H](COS(N)(=O)=O)[C@H]4F)cc32)c2cc(Cl)ccc21] | 624 |
| 458 | 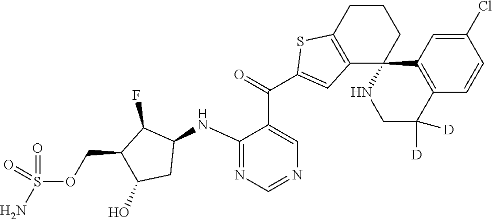 [SMILES: [2H]C1([2H])CN[C@]2(CCCc3sc(C(=O)c4cncnc4N[C@H]4C[C@H](O)[C@@H](COS(N)(=O)=O)[C@H]4F)cc32)c2cc(Cl)ccc21] | 624 |
| 459 | 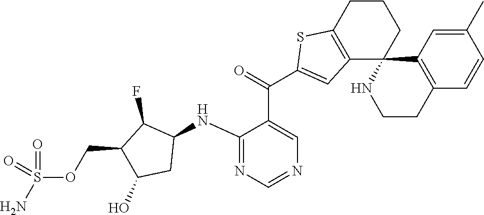 [SMILES: Cc1ccc2c(c1)[C@@]1(CCCc3sc(C(=O)c4cncnc4N[C@H]4C[C@H](O)[C@@H](COS(N)(=O)=O)[C@H]4F)cc31)NCC2] | 602 |
| 460 | 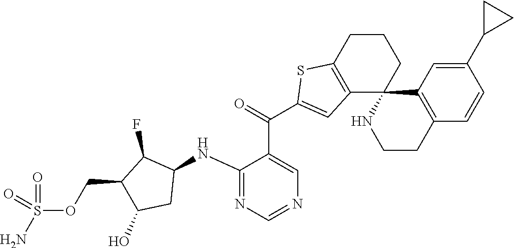 [SMILES: NS(=O)(=O)OC[C@H]1[C@@H](F)[C@@H](Nc2ncncc2C(=O)c2cc3c(s2)CCC[C@]32NCCc3ccc(C4CC4)cc32)C[C@@H]1O] | 628 |
| 461 | 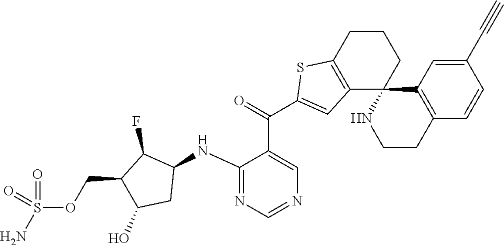 [SMILES: C#Cc1ccc2c(c1)[C@@]1(CCCc3sc(C(=O)c4cncnc4N[C@H]4C[C@H](O)[C@@H](COS(N)(=O)=O)[C@H]4F)cc31)NCC2] | 612 |
| 462 | 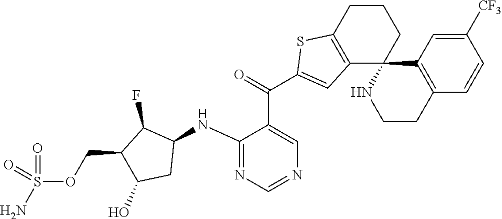 [SMILES: NS(=O)(=O)OC[C@H]1[C@@H](F)[C@@H](Nc2ncncc2C(=O)c2cc3c(s2)CCC[C@]32NCCc3ccc(C(F)(F)F)cc32)C[C@@H]1O] | 656 |
| 463 | 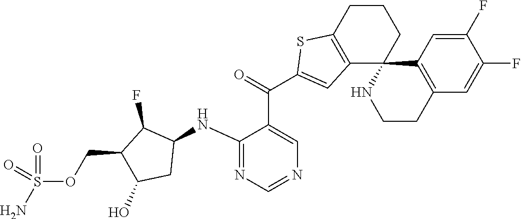 [SMILES: NS(=O)(=O)OC[C@H]1[C@@H](F)[C@@H](Nc2ncncc2C(=O)c2cc3c(s2)CCC[C@]32NCCc3cc(F)c(F)cc32)C[C@@H]1O] | 624 |
| 464 | 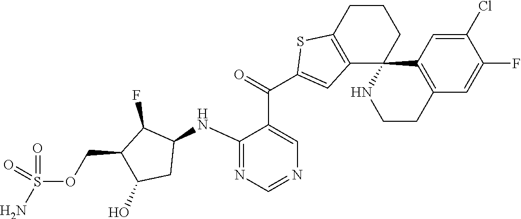 [SMILES: NS(=O)(=O)OC[C@H]1[C@@H](F)[C@@H](Nc2ncncc2C(=O)c2cc3c(s2)CCC[C@]32NCCc3cc(F)c(Cl)cc32)C[C@@H]1O] | 640 |
| 465 | 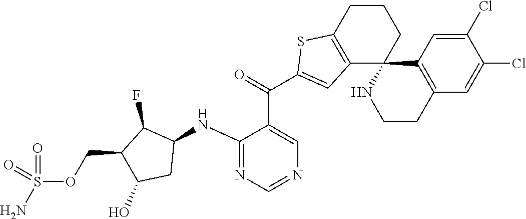 [SMILES: NS(=O)(=O)OC[C@H]1[C@@H](F)[C@@H](Nc2ncncc2C(=O)c2cc3c(s2)CCC[C@]32NCCc3cc(Cl)c(Cl)cc32)C[C@@H]1O] | 656 |
| 466 | 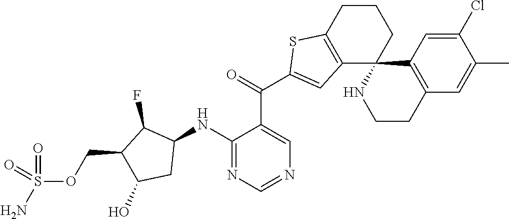 [SMILES: Cc1cc2c(cc1Cl)[C@@]1(CCCc3sc(C(=O)c4cncnc4N[C@H]4C[C@H](O)[C@@H](COS(N)(=O)=O)[C@H]4F)cc31)NCC2] | 636 |
| 467 | 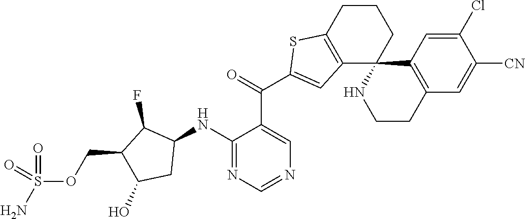 [SMILES: N#Cc1cc2c(cc1Cl)[C@@]1(CCCc3sc(C(=O)c4cncnc4N[C@H]4C[C@H](O)[C@@H](COS(N)(=O)=O)[C@H]4F)cc31)NCC2] | 647 |
| 468 | 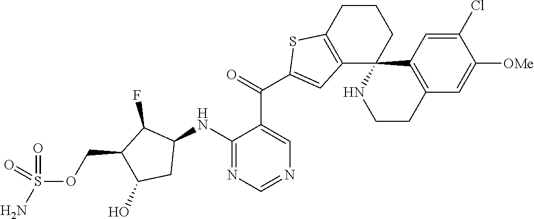 [SMILES: COc1cc2c(cc1Cl)[C@@]1(CCCc3sc(C(=O)c4cncnc4N[C@H]4C[C@H](O)[C@@H](COS(N)(=O)=O)[C@H]4F)cc31)NCC2] | 652 |
| 469 | 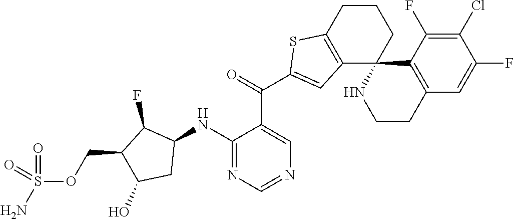 [SMILES: NS(=O)(=O)OC[C@H]1[C@@H](F)[C@@H](Nc2ncncc2C(=O)c2cc3c(s2)CCC[C@]32NCCc3cc(F)c(Cl)c(F)c32)C[C@@H]1O] | 658 |
| 470 | 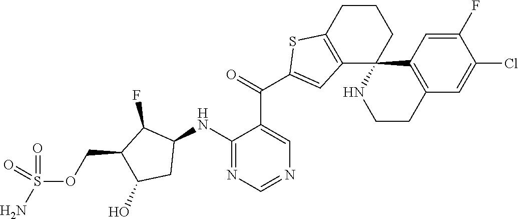 [SMILES: NS(=O)(=O)OC[C@H]1[C@@H](F)[C@@H](Nc2ncncc2C(=O)c2cc3c(s2)CCC[C@]32NCCc3cc(Cl)c(F)cc32)C[C@@H]1O] | 640 |
| 471 | 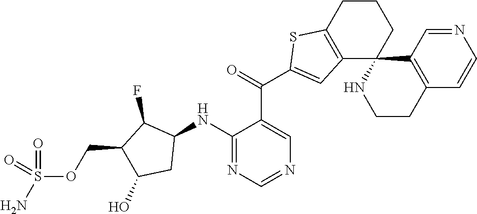 [SMILES: NS(=O)(=O)OC[C@H]1[C@@H](F)[C@@H](Nc2ncncc2C(=O)c2cc3c(s2)CCC[C@]32NCCc3ccncc32)C[C@@H]1O] | 589 |
| 472 | 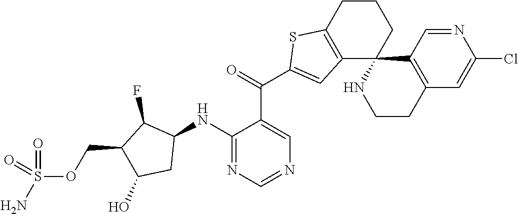 [SMILES: NS(=O)(=O)OC[C@H]1[C@@H](F)[C@@H](Nc2ncncc2C(=O)c2cc3c(s2)CCC[C@]32NCCc3cc(Cl)ncc32)C[C@@H]1O] | 623 |
| 473 | 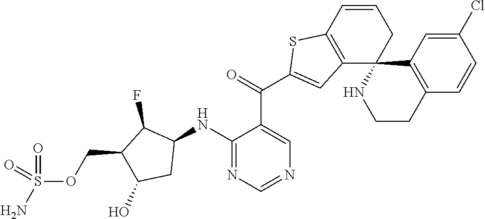 [SMILES: NS(=O)(=O)OC[C@H]1[C@@H](F)[C@@H](Nc2ncncc2C(=O)c2cc3c(s2)C=CC[C@]32NCCc3ccc(Cl)cc32)C[C@@H]1O] | 620 |
| 474 | 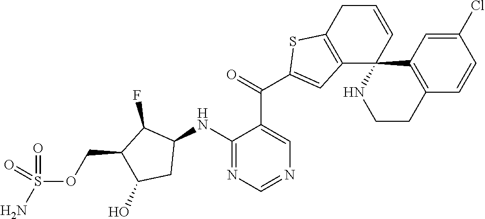 [SMILES: NS(=O)(=O)OC[C@H]1[C@@H](F)[C@@H](Nc2ncncc2C(=O)c2cc3c(s2)CC=C[C@]32NCCc3ccc(Cl)cc32)C[C@@H]1O] | 620 |
| 475 | 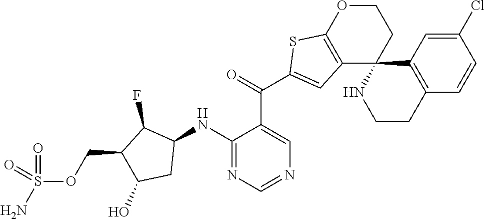 [SMILES: NS(=O)(=O)OC[C@H]1[C@@H](F)[C@@H](Nc2ncncc2C(=O)c2cc3c(s2)OCC[C@]32NCCc3ccc(Cl)cc32)C[C@@H]1O] | 624 |
| 476 | 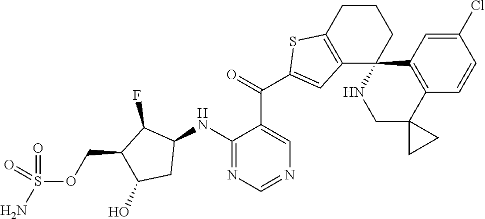 [SMILES: NS(=O)(=O)OC[C@H]1[C@@H](F)[C@@H](Nc2ncncc2C(=O)c2cc3c(s2)CCC[C@]32NCC3(CC3)c3ccc(Cl)cc32)C[C@@H]1O] | 648 |
| 477 | 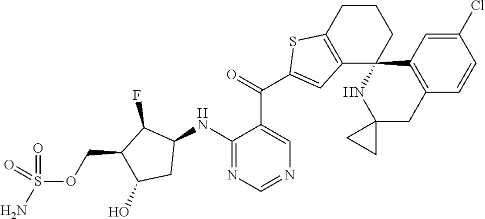 [SMILES: NS(=O)(=O)OC[C@H]1[C@@H](F)[C@@H](Nc2ncncc2C(=O)c2cc3c(s2)CCC[C@]32NC3(CC3)Cc3ccc(Cl)cc32)C[C@@H]1O] | 648 |
| 478 | 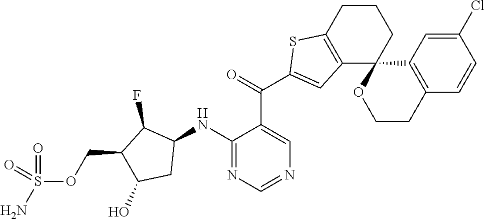 [SMILES: NS(=O)(=O)OC[C@H]1[C@@H](F)[C@@H](Nc2ncncc2C(=O)c2cc3c(s2)CCC[C@@]32OCCc3ccc(Cl)cc32)C[C@@H]1O] | 623 |
| 479 | 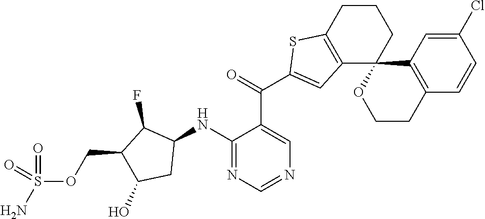 [SMILES: NS(=O)(=O)OC[C@H]1[C@@H](F)[C@@H](Nc2ncncc2C(=O)c2cc3c(s2)CCC[C@]32OCCc3ccc(Cl)cc32)C[C@@H]1O] | 623 |
| 480 | 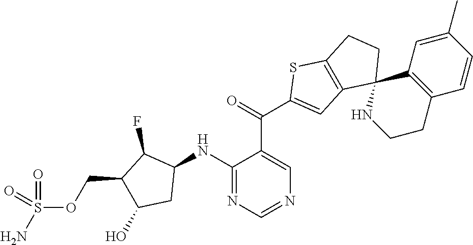 [SMILES: Cc1ccc2c(c1)[C@@]1(CCc3sc(C(=O)c4cncnc4N[C@H]4C[C@H](O)[C@@H](COS(N)(=O)=O)[C@H]4F)cc31)NCC2] | 588 |
| 481 | 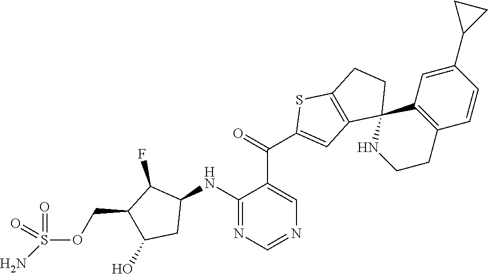 [SMILES: NS(=O)(=O)OC[C@H]1[C@@H](F)[C@@H](Nc2ncncc2C(=O)c2cc3c(s2)CC[C@]32NCCc3ccc(C4CC4)cc32)C[C@@H]1O] | 614 |
| 482 | 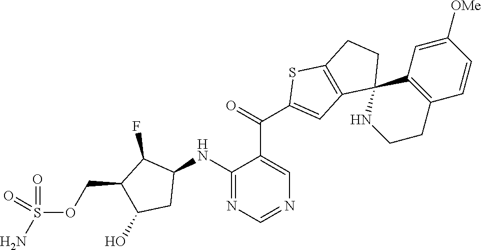 [SMILES: COc1ccc2c(c1)[C@@]1(CCc3sc(C(=O)c4cncnc4N[C@H]4C[C@H](O)[C@@H](COS(N)(=O)=O)[C@H]4F)cc31)NCC2] | 604 |
| 483 | 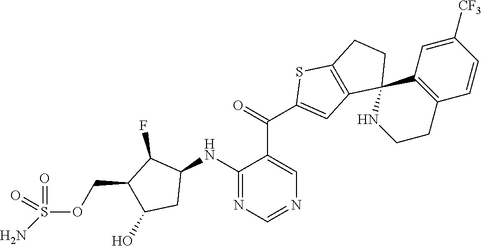 [SMILES: NS(=O)(=O)OC[C@H]1[C@@H](F)[C@@H](Nc2ncncc2C(=O)c2cc3c(s2)CC[C@]32NCCc3ccc(C(F)(F)F)cc32)C[C@@H]1O] | 642 |
| 484 | 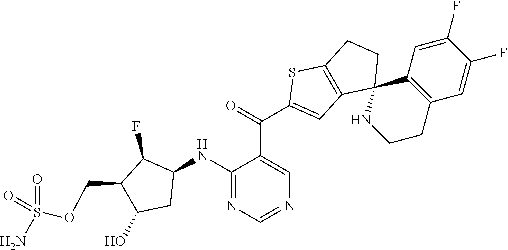 [SMILES: NS(=O)(=O)OC[C@H]1[C@@H](F)[C@@H](Nc2ncncc2C(=O)c2cc3c(s2)CC[C@]32NCCc3cc(F)c(F)cc32)C[C@@H]1O] | 610 |
| 485 | 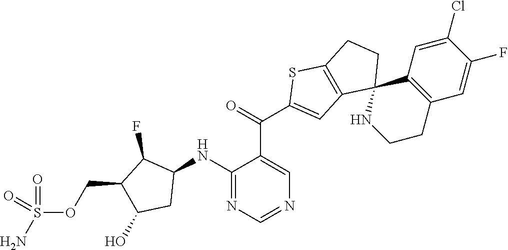 [SMILES: NS(=O)(=O)OC[C@H]1[C@@H](F)[C@@H](Nc2ncncc2C(=O)c2cc3c(s2)CC[C@]32NCCc3cc(F)c(Cl)cc32)C[C@@H]1O] | 626 |
| 486 | 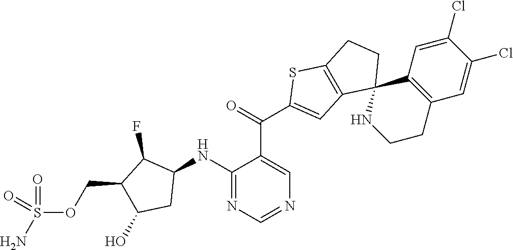 [SMILES: NS(=O)(=O)OC[C@H]1[C@@H](F)[C@@H](Nc2ncncc2C(=O)c2cc3c(s2)CC[C@]32NCCc3cc(Cl)c(Cl)cc32)C[C@@H]1O] | 642 |
| 487 | 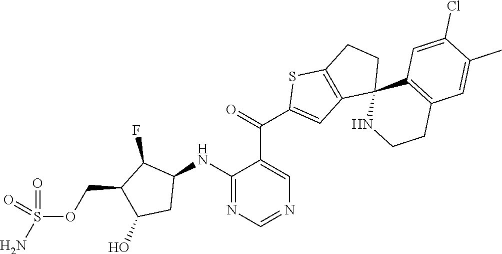 [SMILES: Cc1cc2c(cc1Cl)[C@@]1(CCc3sc(C(=O)c4cncnc4N[C@H]4C[C@H](O)[C@@H](COS(N)(=O)=O)[C@H]4F)cc31)NCC2] | 622 |
| 488 | 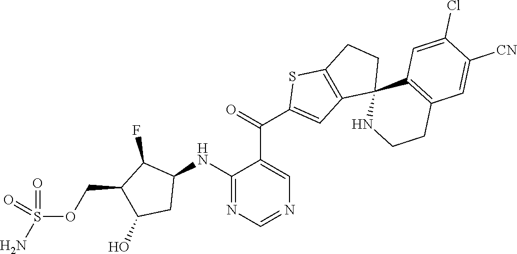 [SMILES: N#Cc1cc2c(cc1Cl)[C@@]1(CCc3sc(C(=O)c4cncnc4N[C@H]4C[C@H](O)[C@@H](COS(N)(=O)=O)[C@H]4F)cc31)NCC2] | 633 |
| 489 | 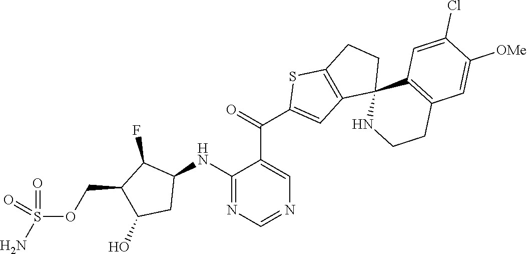 [SMILES: COc1cc2c(cc1Cl)[C@@]1(CCc3sc(C(=O)c4cncnc4N[C@H]4C[C@H](O)[C@@H](COS(N)(=O)=O)[C@H]4F)cc31)NCC2] | 638 |
| 490 | 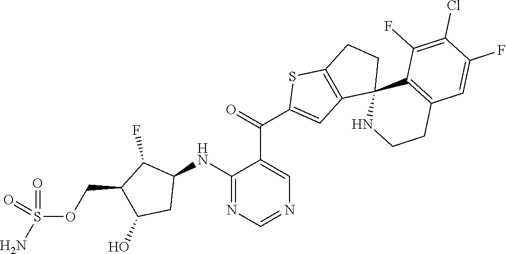 [SMILES: NS(=O)(=O)OC[C@H]1[C@H](F)[C@@H](Nc2ncncc2C(=O)c2cc3c(s2)CC[C@]32NCCc3cc(F)c(Cl)c(F)c32)C[C@@H]1O] | 644 |
| 491 | 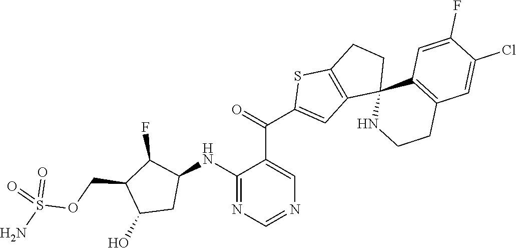 [SMILES: NS(=O)(=O)OC[C@H]1[C@@H](F)[C@@H](Nc2ncncc2C(=O)c2cc3c(s2)CC[C@]32NCCc3cc(Cl)c(F)cc32)C[C@@H]1O] | 626 |
| 492 | 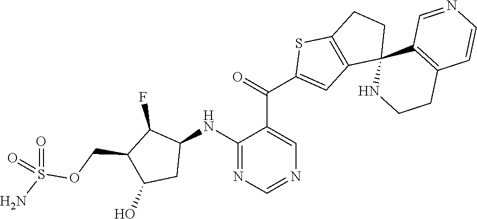 [SMILES: NS(=O)(=O)OC[C@H]1[C@@H](F)[C@@H](Nc2ncncc2C(=O)c2cc3c(s2)CC[C@]32NCCc3ccncc32)C[C@@H]1O] | 575 |
| 493 | 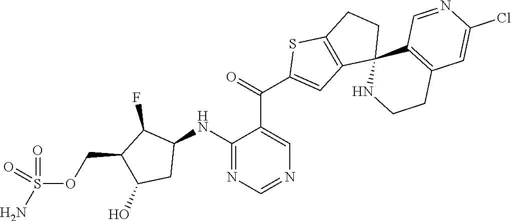 [SMILES: NS(=O)(=O)OC[C@H]1[C@@H](F)[C@@H](Nc2ncncc2C(=O)c2cc3c(s2)CC[C@]32NCCc3cc(Cl)ncc32)C[C@@H]1O] | 609 |
| 494 | 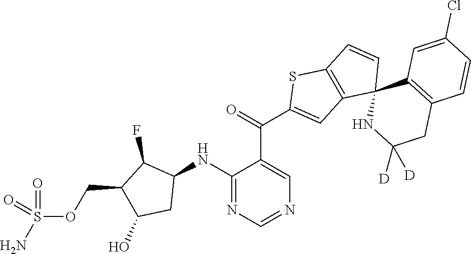 [SMILES: [2H]C1([2H])Cc2ccc(Cl)cc2[C@@]2(C=Cc3sc(C(=O)c4cncnc4N[C@H]4C[C@H](O)[C@@H](COS(N)(=O)=O)[C@H]4F)cc32)N1] | 608 |
| 495 | 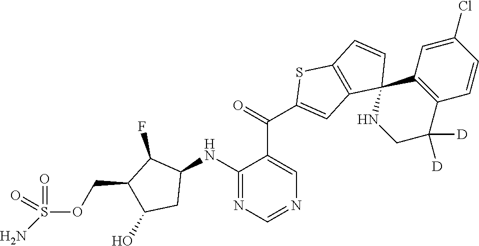 [SMILES: [2H]C1([2H])CN[C@]2(C=Cc3sc(C(=O)c4cncnc4N[C@H]4C[C@H](O)[C@@H](COS(N)(=O)=O)[C@H]4F)cc32)c2cc(Cl)ccc21] | 608 |
| 496 | 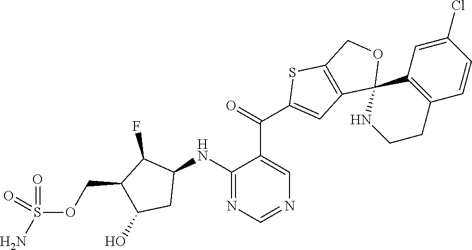 [SMILES: NS(=O)(=O)OC[C@H]1[C@@H](F)[C@@H](Nc2ncncc2C(=O)c2cc3c(s2)CO[C@]32NCCc3ccc(Cl)cc32)C[C@@H]1O] | 610 |
| 497 | 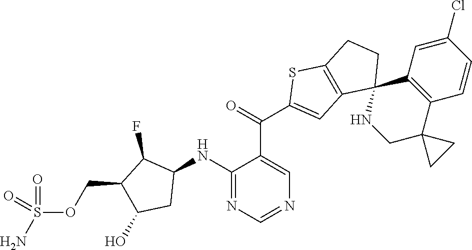 [SMILES: NS(=O)(=O)OC[C@H]1[C@@H](F)[C@@H](Nc2ncncc2C(=O)c2cc3c(s2)CC[C@]32NCC3(CC3)c3ccc(Cl)cc32)C[C@@H]1O] | 634 |
| 498 | 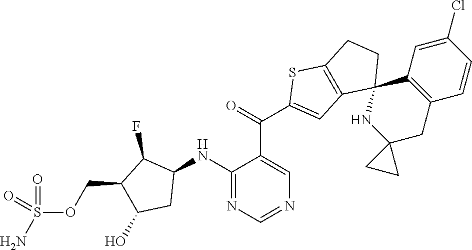 [SMILES: NS(=O)(=O)OC[C@H]1[C@@H](F)[C@@H](Nc2ncncc2C(=O)c2cc3c(s2)CC[C@]32NC3(CC3)Cc3ccc(Cl)cc32)C[C@@H]1O] | 634 |
| 499 | 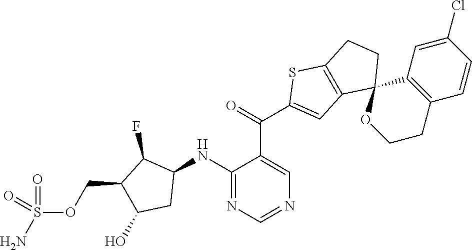 [SMILES: NS(=O)(=O)OC[C@H]1[C@@H](F)[C@@H](Nc2ncncc2C(=O)c2cc3c(s2)CC[C@@]32OCCc3ccc(Cl)cc32)C[C@@H]1O] | 609 |
| 500 | 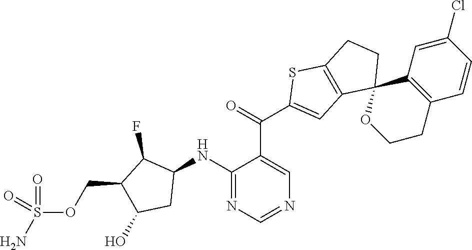 [SMILES: NS(=O)(=O)OC[C@H]1[C@@H](F)[C@@H](Nc2ncncc2C(=O)c2cc3c(s2)CC[C@]32OCCc3ccc(Cl)cc32)C[C@@H]1O] | 609 |
| 501 | 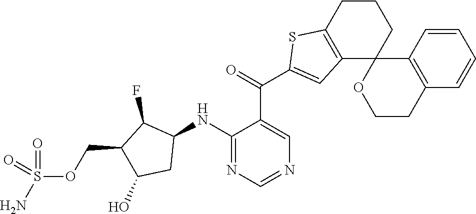 [SMILES: NS(=O)(=O)OC[C@H]1[C@@H](F)[C@@H](Nc2ncncc2C(=O)c2cc3c(s2)CCCC32OCCc3ccccc32)C[C@@H]1O] | 589 |
| 502 | 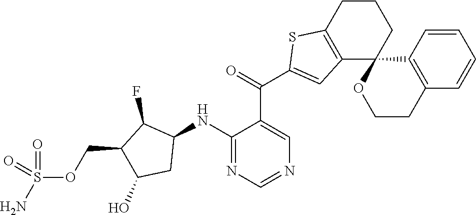 [SMILES: NS(=O)(=O)OC[C@H]1[C@@H](F)[C@@H](Nc2ncncc2C(=O)c2cc3c(s2)CCC[C@@]32OCCc3ccccc32)C[C@@H]1O] | 589 |
| 503 | 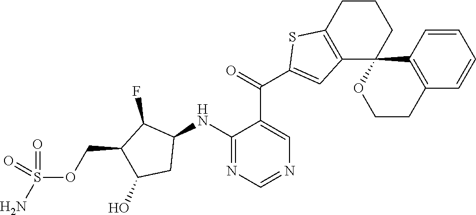 [SMILES: NS(=O)(=O)OC[C@H]1[C@@H](F)[C@@H](Nc2ncncc2C(=O)c2cc3c(s2)CCC[C@]32OCCc3ccccc32)C[C@@H]1O] | 589 |
| 504 | 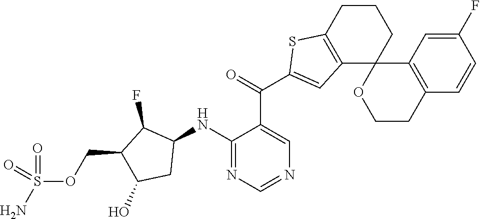 [SMILES: NS(=O)(=O)OC[C@H]1[C@@H](F)[C@@H](Nc2ncncc2C(=O)c2cc3c(s2)CCCC32OCCc3ccc(F)cc32)C[C@@H]1O] | 607 |
| 505 | 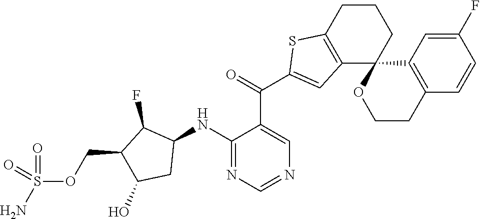 [SMILES: NS(=O)(=O)OC[C@H]1[C@@H](F)[C@@H](Nc2ncncc2C(=O)c2cc3c(s2)CCC[C@@]32OCCc3ccc(F)cc32)C[C@@H]1O] | 607 |
| 506 | 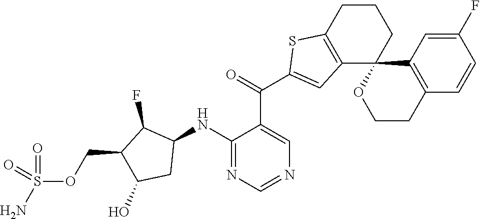 [SMILES: NS(=O)(=O)OC[C@H]1[C@@H](F)[C@@H](Nc2ncncc2C(=O)c2cc3c(s2)CCC[C@]32OCCc3ccc(F)cc32)C[C@@H]1O] | 607 |
| 507 | 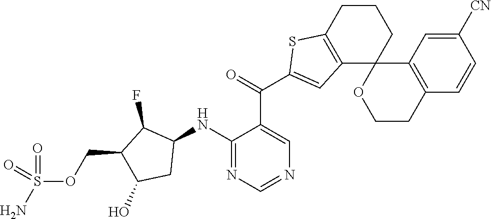 [SMILES: N#Cc1ccc2c(c1)C1(CCCc3sc(C(=O)c4cncnc4N[C@H]4C[C@H](O)[C@@H](COS(N)(=O)=O)[C@H]4F)cc31)OCC2] | 614 |
| 508 | 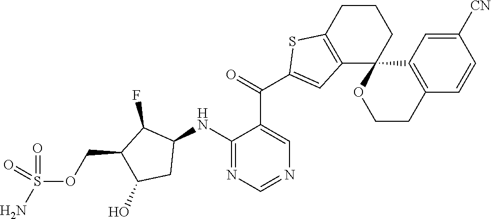 [SMILES: N#Cc1ccc2c(c1)[C@]1(CCCc3sc(C(=O)c4cncnc4N[C@H]4C[C@H](O)[C@@H](COS(N)(=O)=O)[C@H]4F)cc31)OCC2] | 614 |
| 509 | 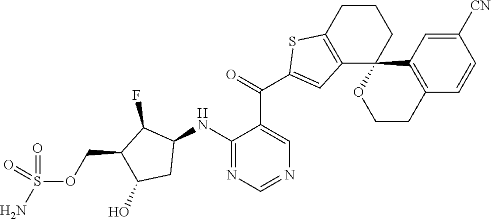 [SMILES: N#Cc1ccc2c(c1)[C@@]1(CCCc3sc(C(=O)c4cncnc4N[C@H]4C[C@H](O)[C@@H](COS(N)(=O)=O)[C@H]4F)cc31)OCC2] | 614 |
| 510 | 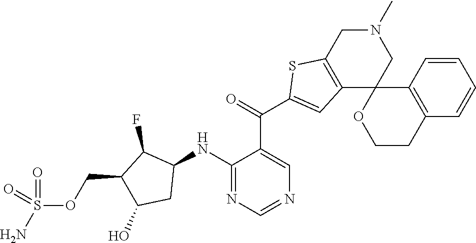 [SMILES: CN1Cc2sc(C(=O)c3cncnc3N[C@H]3C[C@H](O)[C@@H](COS(N)(=O)=O)[C@H]3F)cc2C2(C1)OCCc1ccccc12] | 604 |
| 511 | 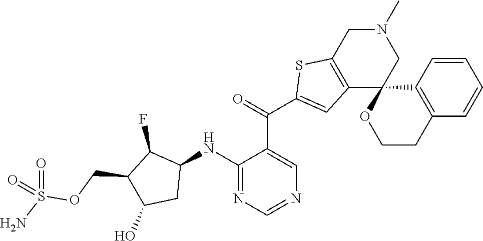 [SMILES: CN1Cc2sc(C(=O)c3cncnc3N[C@H]3C[C@H](O)[C@@H](COS(N)(=O)=O)[C@H]3F)cc2[C@@]2(C1)OCCc1ccccc12] | 604 |
| 512 | 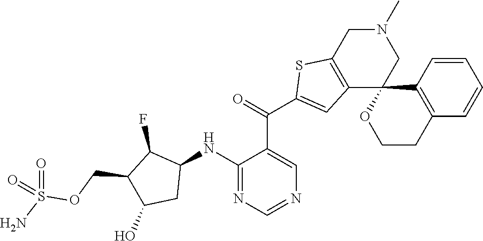 [SMILES: CN1Cc2sc(C(=O)c3cncnc3N[C@H]3C[C@H](O)[C@@H](COS(N)(=O)=O)[C@H]3F)cc2[C@]2(C1)OCCc1ccccc12] | 604 |
| 513 | 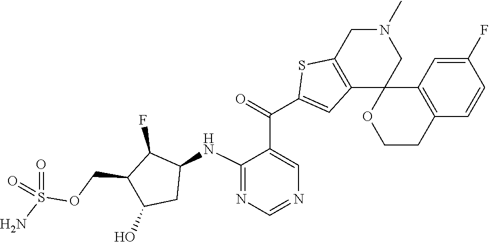 [SMILES: CN1Cc2sc(C(=O)c3cncnc3N[C@H]3C[C@H](O)[C@@H](COS(N)(=O)=O)[C@H]3F)cc2C2(C1)OCCc1ccc(F)cc12] | 622 |
| 514 | 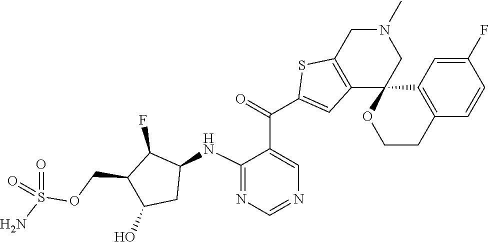 [SMILES: CN1Cc2sc(C(=O)c3cncnc3N[C@H]3C[C@H](O)[C@@H](COS(N)(=O)=O)[C@H]3F)cc2[C@@]2(C1)OCCc1ccc(F)cc12] | 622 |
| 515 | 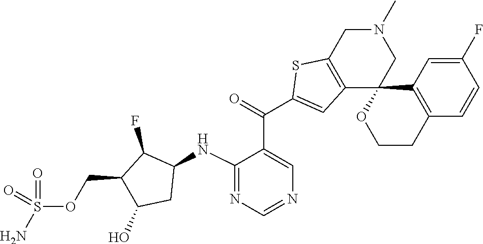 [SMILES: CN1Cc2sc(C(=O)c3cncnc3N[C@H]3C[C@H](O)[C@@H](COS(N)(=O)=O)[C@H]3F)cc2[C@]2(C1)OCCc1ccc(F)cc12] | 622 |
| 516 | 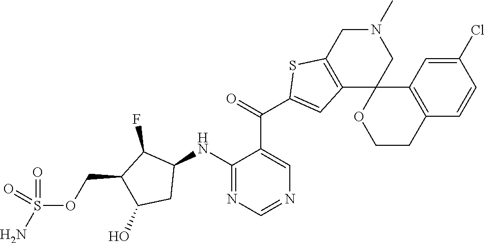 [SMILES: CN1Cc2sc(C(=O)c3cncnc3N[C@H]3C[C@H](O)[C@@H](COS(N)(=O)=O)[C@H]3F)cc2C2(C1)OCCc1ccc(Cl)cc12] | 638 |
| 517 | 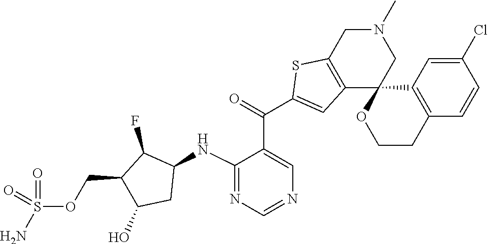 [SMILES: CN1Cc2sc(C(=O)c3cncnc3N[C@H]3C[C@H](O)[C@@H](COS(N)(=O)=O)[C@H]3F)cc2[C@@]2(C1)OCCc1ccc(Cl)cc12] | 638 |
| 518 | 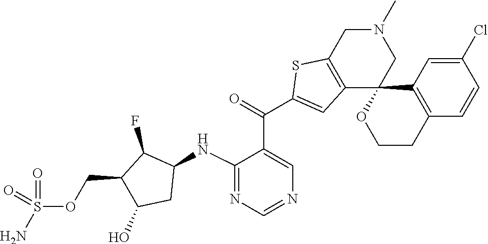 [SMILES: CN1Cc2sc(C(=O)c3cncnc3N[C@H]3C[C@H](O)[C@@H](COS(N)(=O)=O)[C@H]3F)cc2[C@]2(C1)OCCc1ccc(Cl)cc12] | 638 |
| 519 | 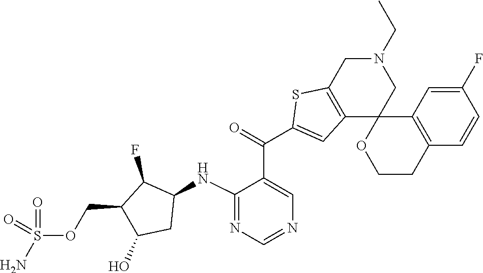 [SMILES: CCN1Cc2sc(C(=O)c3cncnc3N[C@H]3C[C@H](O)[C@@H](COS(N)(=O)=O)[C@H]3F)cc2C2(C1)OCCc1ccc(F)cc12] | 636 |
| 520 | 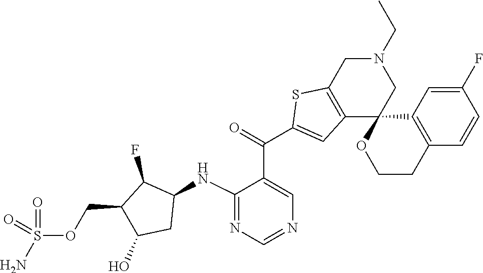 [SMILES: CCN1Cc2sc(C(=O)c3cncnc3N[C@H]3C[C@H](O)[C@@H](COS(N)(=O)=O)[C@H]3F)cc2[C@@]2(C1)OCCc1ccc(F)cc12] | 636 |
| 521 | 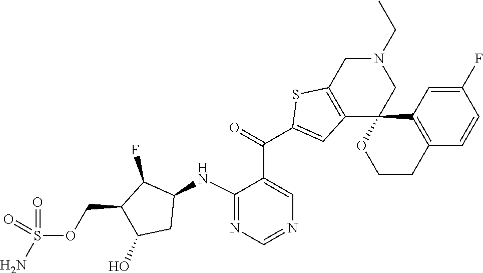 [SMILES: CCN1Cc2sc(C(=O)c3cncnc3N[C@H]3C[C@H](O)[C@@H](COS(N)(=O)=O)[C@H]3F)cc2[C@]2(C1)OCCc1ccc(F)cc12] | 636 |
| 522 | 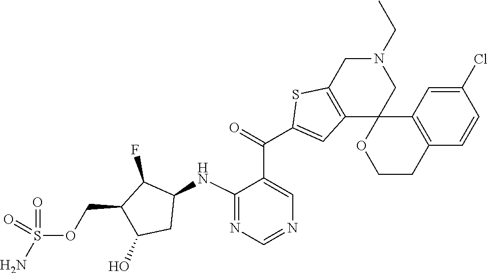 [SMILES: CCN1Cc2sc(C(=O)c3cncnc3N[C@H]3C[C@H](O)[C@@H](COS(N)(=O)=O)[C@H]3F)cc2C2(C1)OCCc1ccc(Cl)cc12] | 652 |
| 523 | 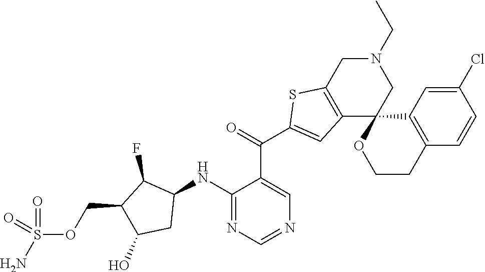 [SMILES: CCN1Cc2sc(C(=O)c3cncnc3N[C@H]3C[C@H](O)[C@@H](COS(N)(=O)=O)[C@H]3F)cc2[C@@]2(C1)OCCc1ccc(Cl)cc12] | 652 |
| 524 | 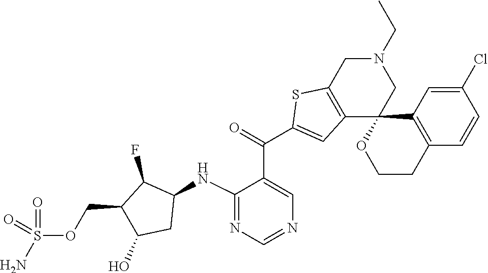 [SMILES: CCN1Cc2sc(C(=O)c3cncnc3N[C@H]3C[C@H](O)[C@@H](COS(N)(=O)=O)[C@H]3F)cc2[C@]2(C1)OCCc1ccc(Cl)cc12] | 652 |
| 525 | 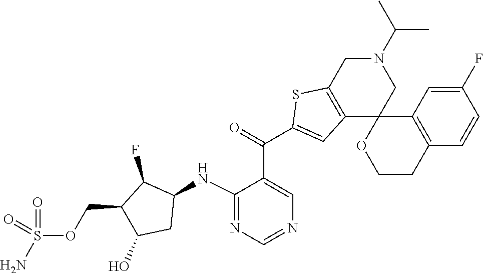 [SMILES: CC(C)N1Cc2sc(C(=O)c3cncnc3N[C@H]3C[C@H](O)[C@@H](COS(N)(=O)=O)[C@H]3F)cc2C2(C1)OCCc1ccc(F)cc12] | 650 |
| 526 | 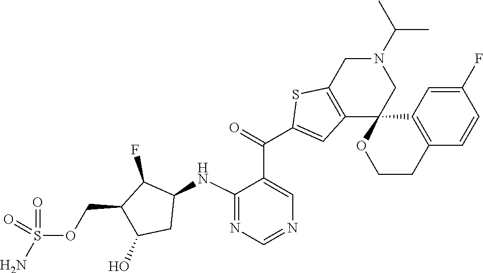 [SMILES: CC(C)N1Cc2sc(C(=O)c3cncnc3N[C@H]3C[C@H](O)[C@@H](COS(N)(=O)=O)[C@H]3F)cc2[C@@]2(C1)OCCc1ccc(F)cc12] | 650 |
| 527 | 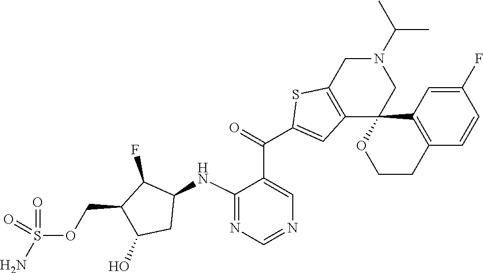 [SMILES: CC(C)N1Cc2sc(C(=O)c3cncnc3N[C@H]3C[C@H](O)[C@@H](COS(N)(=O)=O)[C@H]3F)cc2[C@]2(C1)OCCc1ccc(F)cc12] | 650 |
| 528 | 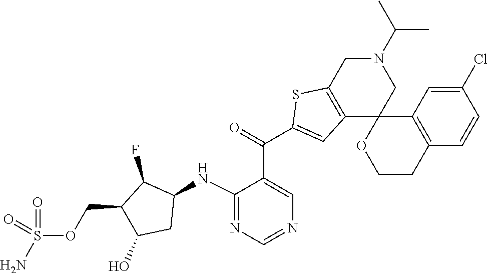 [SMILES: CC(C)N1Cc2sc(C(=O)c3cncnc3N[C@H]3C[C@H](O)[C@@H](COS(N)(=O)=O)[C@H]3F)cc2C2(C1)OCCc1ccc(Cl)cc12] | 666 |
| 529 | 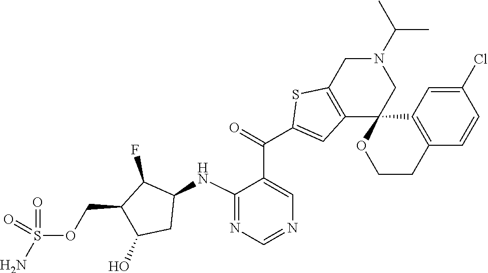 [SMILES: CC(C)N1Cc2sc(C(=O)c3cncnc3N[C@H]3C[C@H](O)[C@@H](COS(N)(=O)=O)[C@H]3F)cc2[C@@]2(C1)OCCc1ccc(Cl)cc12] | 666 |
| 530 | 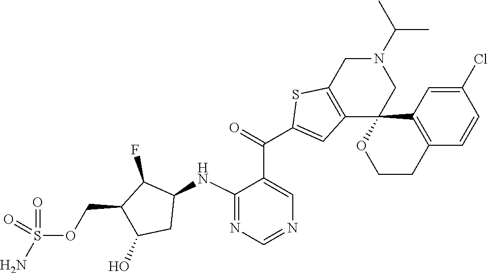 [SMILES: CC(C)N1Cc2sc(C(=O)c3cncnc3N[C@H]3C[C@H](O)[C@@H](COS(N)(=O)=O)[C@H]3F)cc2[C@]2(C1)OCCc1ccc(Cl)cc12] | 666 |
| 531 | 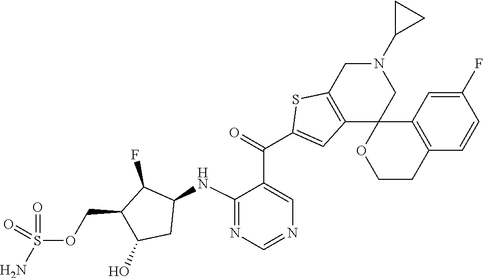 [SMILES: NS(=O)(=O)OC[C@H]1[C@@H](F)[C@@H](Nc2ncncc2C(=O)c2cc3c(s2)CN(C2CC2)CC32OCCc3ccc(F)cc32)C[C@@H]1O] | 648 |
| 532 | 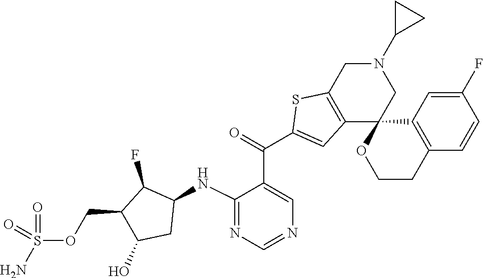 [SMILES: NS(=O)(=O)OC[C@H]1[C@@H](F)[C@@H](Nc2ncncc2C(=O)c2cc3c(s2)CN(C2CC2)C[C@@]32OCCc3ccc(F)cc32)C[C@@H]1O] | 648 |
| 533 | 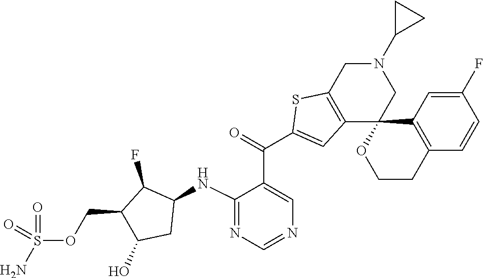 [SMILES: NS(=O)(=O)OC[C@H]1[C@@H](F)[C@@H](Nc2ncncc2C(=O)c2cc3c(s2)CN(C2CC2)C[C@]32OCCc3ccc(F)cc32)C[C@@H]1O] | 648 |
| 534 | 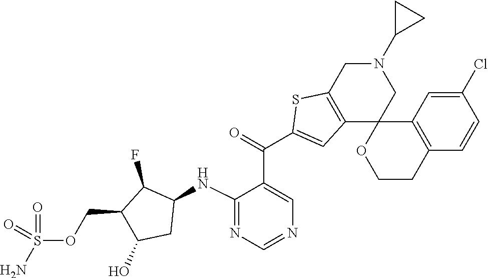 [SMILES: NS(=O)(=O)OC[C@H]1[C@@H](F)[C@@H](Nc2ncncc2C(=O)c2cc3c(s2)CN(C2CC2)CC32OCCc3ccc(Cl)cc32)C[C@@H]1O] | 664 |
| 535 | 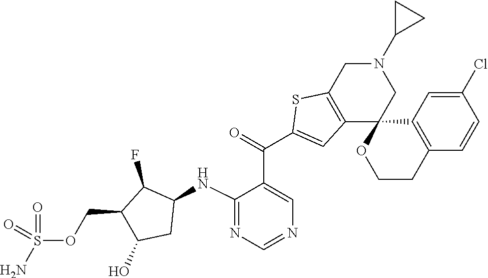 [SMILES: NS(=O)(=O)OC[C@H]1[C@@H](F)[C@@H](Nc2ncncc2C(=O)c2cc3c(s2)CN(C2CC2)C[C@@]32OCCc3ccc(Cl)cc32)C[C@@H]1O] | 664 |
| 536 | 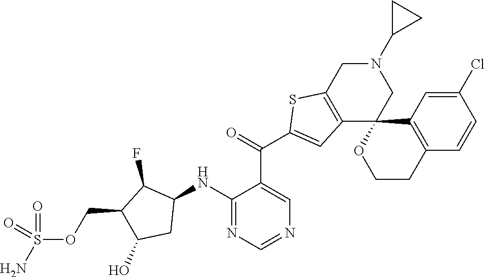 [SMILES: NS(=O)(=O)OC[C@H]1[C@@H](F)[C@@H](Nc2ncncc2C(=O)c2cc3c(s2)CN(C2CC2)C[C@]32OCCc3ccc(Cl)cc32)C[C@@H]1O] | 664 |
| 537 | 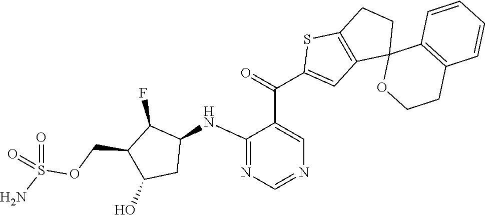 [SMILES: NS(=O)(=O)OC[C@H]1[C@@H](F)[C@@H](Nc2ncncc2C(=O)c2cc3c(s2)CCC32OCCc3ccccc32)C[C@@H]1O] | 575 |
| 538 | 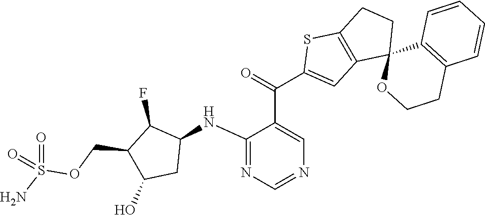 [SMILES: NS(=O)(=O)OC[C@H]1[C@@H](F)[C@@H](Nc2ncncc2C(=O)c2cc3c(s2)CC[C@@]32OCCc3ccccc32)C[C@@H]1O] | 575 |
| 539 | 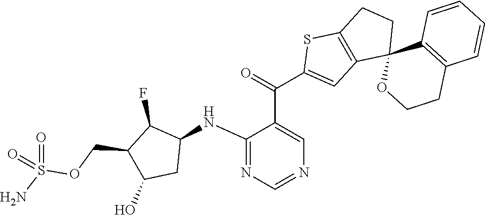 [SMILES: NS(=O)(=O)OC[C@H]1[C@@H](F)[C@@H](Nc2ncncc2C(=O)c2cc3c(s2)CC[C@]32OCCc3ccccc32)C[C@@H]1O] | 575 |
| 540 | 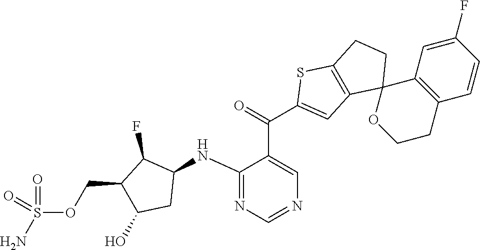 [SMILES: NS(=O)(=O)OC[C@H]1[C@@H](F)[C@@H](Nc2ncncc2C(=O)c2cc3c(s2)CCC32OCCc3ccc(F)cc32)C[C@@H]1O] | 593 |
| 541 | 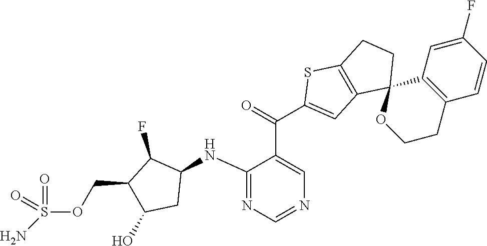 [SMILES: NS(=O)(=O)OC[C@H]1[C@@H](F)[C@@H](Nc2ncncc2C(=O)c2cc3c(s2)CC[C@@]32OCCc3ccc(F)cc32)C[C@@H]1O] | 593 |
| 542 | 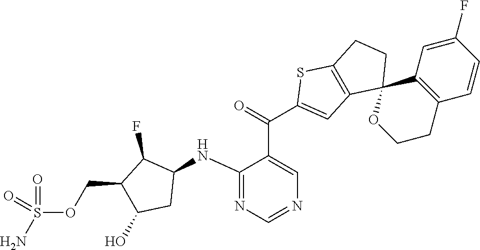 [SMILES: NS(=O)(=O)OC[C@H]1[C@@H](F)[C@@H](Nc2ncncc2C(=O)c2cc3c(s2)CC[C@]32OCCc3ccc(F)cc32)C[C@@H]1O] | 593 |
| 543 | 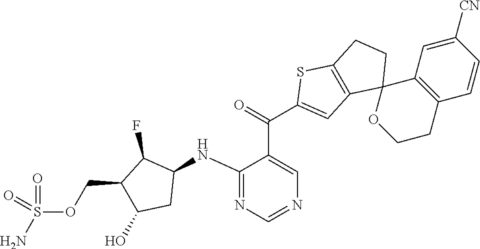 [SMILES: N#Cc1ccc2c(c1)C1(CCc3sc(C(=O)c4cncnc4N[C@H]4C[C@H](O)[C@@H](COS(N)(=O)=O)[C@H]4F)cc31)OCC2] | 600 |
| 544 | 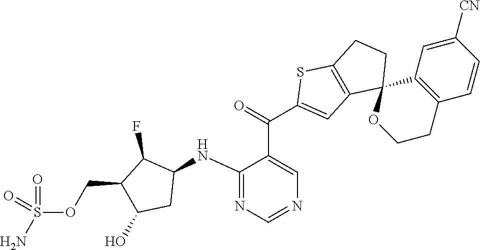 [SMILES: N#Cc1ccc2c(c1)[C@]1(CCc3sc(C(=O)c4cncnc4N[C@H]4C[C@H](O)[C@@H](COS(N)(=O)=O)[C@H]4F)cc31)OCC2] | 600 |
| 545 | 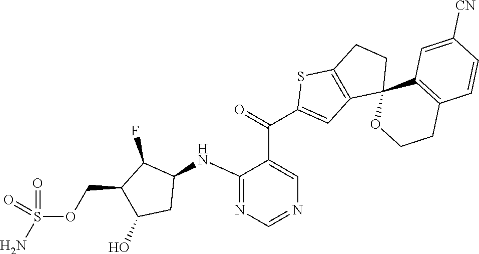 [SMILES: N#Cc1ccc2c(c1)[C@@]1(CCc3sc(C(=O)c4cncnc4N[C@H]4C[C@H](O)[C@@H](COS(N)(=O)=O)[C@H]4F)cc31)OCC2] | 600 |
| 546 | 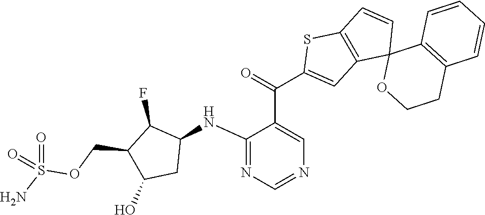 [SMILES: NS(=O)(=O)OC[C@H]1[C@@H](F)[C@@H](Nc2ncncc2C(=O)c2cc3c(s2)C=CC32OCCc3ccccc32)C[C@@H]1O] | 573 |
| 547 | 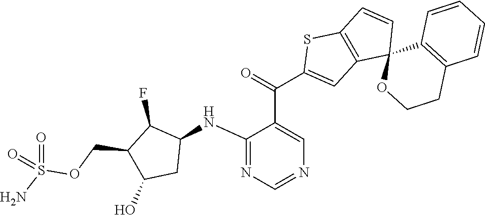 [SMILES: NS(=O)(=O)OC[C@H]1[C@@H](F)[C@@H](Nc2ncncc2C(=O)c2cc3c(s2)C=C[C@@]32OCCc3ccccc32)C[C@@H]1O] | 573 |
| 548 | 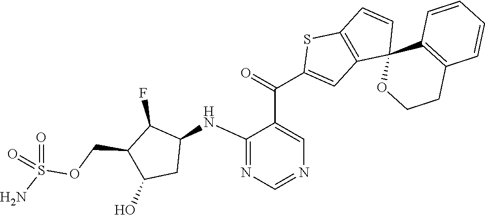 [SMILES: NS(=O)(=O)OC[C@H]1[C@@H](F)[C@@H](Nc2ncncc2C(=O)c2cc3c(s2)C=C[C@]32OCCc3ccccc32)C[C@@H]1O] | 573 |
| 549 | 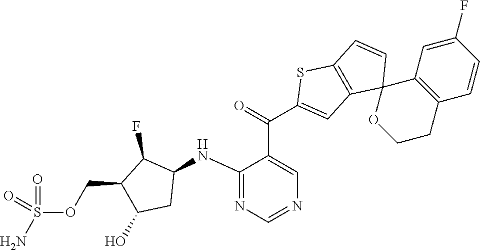 [SMILES: NS(=O)(=O)OC[C@H]1[C@@H](F)[C@@H](Nc2ncncc2C(=O)c2cc3c(s2)C=CC32OCCc3ccc(F)cc32)C[C@@H]1O] | 591 |
| 550 | 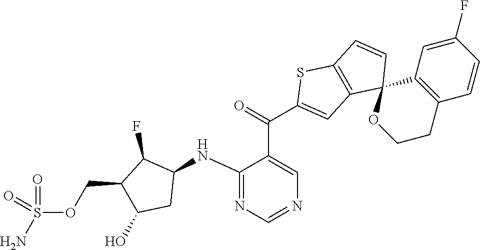 [SMILES: NS(=O)(=O)OC[C@H]1[C@@H](F)[C@@H](Nc2ncncc2C(=O)c2cc3c(s2)C=C[C@@]32OCCc3ccc(F)cc32)C[C@@H]1O] | 591 |
| 551 | 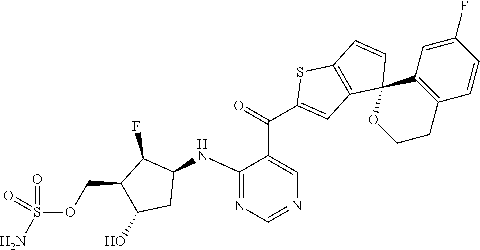 [SMILES: NS(=O)(=O)OC[C@H]1[C@@H](F)[C@@H](Nc2ncncc2C(=O)c2cc3c(s2)C=C[C@]32OCCc3ccc(F)cc32)C[C@@H]1O] | 591 |
| 552 | 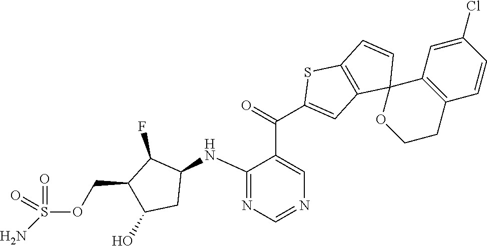 [SMILES: NS(=O)(=O)OC[C@H]1[C@@H](F)[C@@H](Nc2ncncc2C(=O)c2cc3c(s2)C=CC32OCCc3ccc(Cl)cc32)C[C@@H]1O] | 607 |
| 553 | 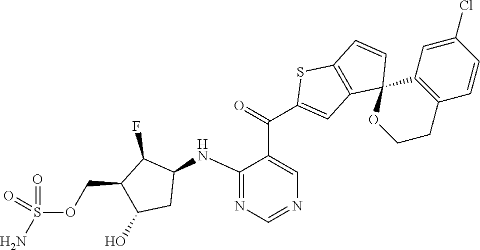 [SMILES: NS(=O)(=O)OC[C@H]1[C@@H](F)[C@@H](Nc2ncncc2C(=O)c2cc3c(s2)C=C[C@@]32OCCc3ccc(Cl)cc32)C[C@@H]1O] | 607 |
| 554 | 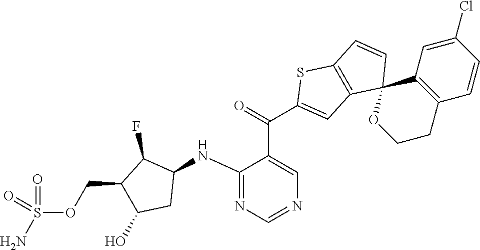 [SMILES: NS(=O)(=O)OC[C@H]1[C@@H](F)[C@@H](Nc2ncncc2C(=O)c2cc3c(s2)C=C[C@]32OCCc3ccc(Cl)cc32)C[C@@H]1O] | 607 |
| 555 | 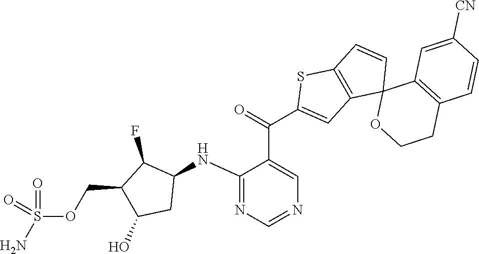 [SMILES: N#Cc1ccc2c(c1)C1(C=Cc3sc(C(=O)c4cncnc4N[C@H]4C[C@H](O)[C@@H](COS(N)(=O)=O)[C@H]4F)cc31)OCC2] | 598 |
| 556 | 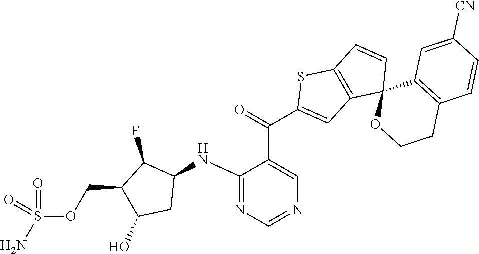 [SMILES: N#Cc1ccc2c(c1)[C@]1(C=Cc3sc(C(=O)c4cncnc4N[C@H]4C[C@H](O)[C@@H](COS(N)(=O)=O)[C@H]4F)cc31)OCC2] | 598 |
| 557 | 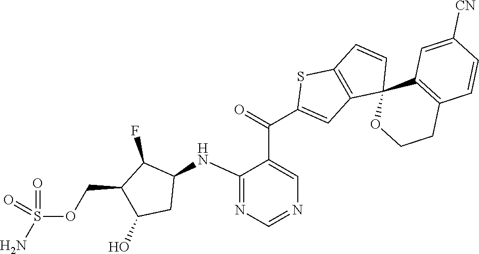 [SMILES: N#Cc1ccc2c(c1)[C@@]1(C=Cc3sc(C(=O)c4cncnc4N[C@H]4C[C@H](O)[C@@H](COS(N)(=O)=O)[C@H]4F)cc31)OCC2] | 598 |
| 558 | 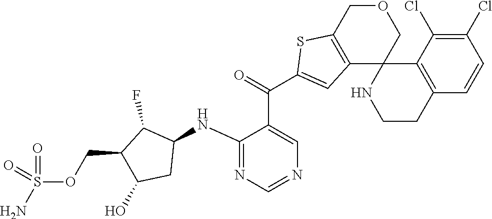 [SMILES: NS(=O)(=O)OC[C@H]1[C@H](F)[C@@H](Nc2ncncc2C(=O)c2cc3c(s2)COCC32NCCc3ccc(Cl)c(Cl)c32)C[C@@H]1O] | 658 |
| 559 | 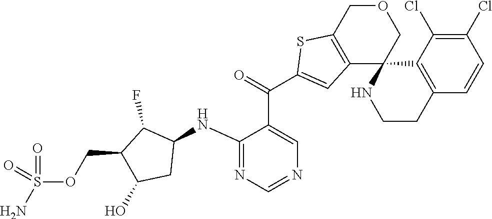 [SMILES: NS(=O)(=O)OC[C@H]1[C@H](F)[C@@H](Nc2ncncc2C(=O)c2cc3c(s2)COC[C@@]32NCCc3ccc(Cl)c(Cl)c32)C[C@@H]1O] | 658 |
| 560 | 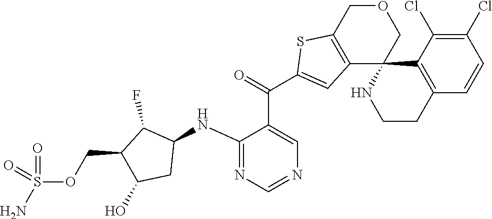 [SMILES: NS(=O)(=O)OC[C@H]1[C@H](F)[C@@H](Nc2ncncc2C(=O)c2cc3c(s2)COC[C@]32NCCc3ccc(Cl)c(Cl)c32)C[C@@H]1O] | 658 |
| 561 | 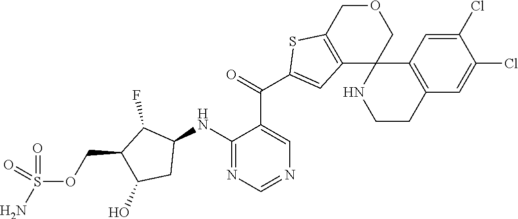 [SMILES: NS(=O)(=O)OC[C@H]1[C@H](F)[C@@H](Nc2ncncc2C(=O)c2cc3c(s2)COCC32NCCc3cc(Cl)c(Cl)cc32)C[C@@H]1O] | 658 |
| 562 | 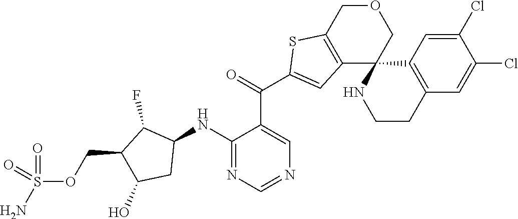 [SMILES: NS(=O)(=O)OC[C@H]1[C@H](F)[C@@H](Nc2ncncc2C(=O)c2cc3c(s2)COC[C@@]32NCCc3cc(Cl)c(Cl)cc32)C[C@@H]1O] | 658 |
| 563 | 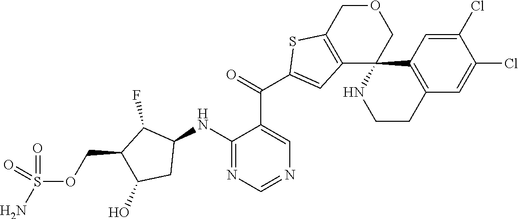 [SMILES: NS(=O)(=O)OC[C@H]1[C@H](F)[C@@H](Nc2ncncc2C(=O)c2cc3c(s2)COC[C@]32NCCc3cc(Cl)c(Cl)cc32)C[C@@H]1O] | 658 |
| 564 | 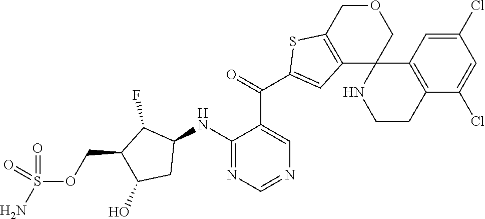 [SMILES: NS(=O)(=O)OC[C@H]1[C@H](F)[C@@H](Nc2ncncc2C(=O)c2cc3c(s2)COCC32NCCc3c(Cl)cc(Cl)cc32)C[C@@H]1O] | 658 |
| 565 | 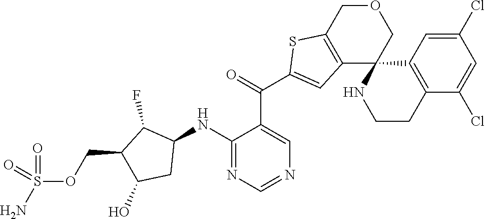 [SMILES: NS(=O)(=O)OC[C@H]1[C@H](F)[C@@H](Nc2ncncc2C(=O)c2cc3c(s2)COC[C@@]32NCCc3c(Cl)cc(Cl)cc32)C[C@@H]1O] | 658 |
| 566 | 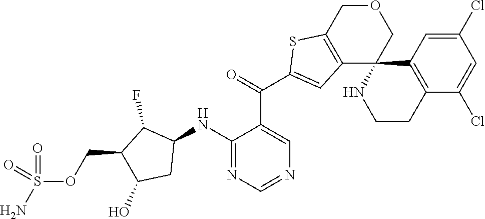 [SMILES: NS(=O)(=O)OC[C@H]1[C@H](F)[C@@H](Nc2ncncc2C(=O)c2cc3c(s2)COC[C@]32NCCc3c(Cl)cc(Cl)cc32)C[C@@H]1O] | 658 |
| 567 | 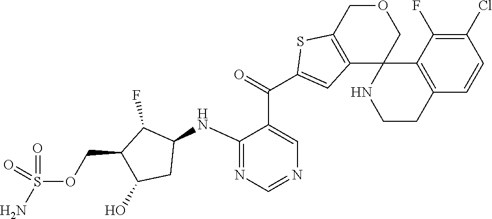 [SMILES: NS(=O)(=O)OC[C@H]1[C@H](F)[C@@H](Nc2ncncc2C(=O)c2cc3c(s2)COCC32NCCc3ccc(Cl)c(F)c32)C[C@@H]1O] | 642 |
| 568 | 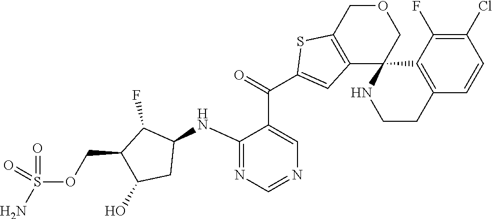 [SMILES: NS(=O)(=O)OC[C@H]1[C@H](F)[C@@H](Nc2ncncc2C(=O)c2cc3c(s2)COC[C@@]32NCCc3ccc(Cl)c(F)c32)C[C@@H]1O] | 642 |
| 569 | 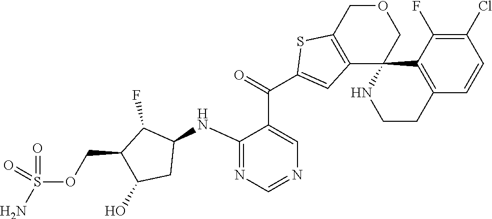 [SMILES: NS(=O)(=O)OC[C@H]1[C@H](F)[C@@H](Nc2ncncc2C(=O)c2cc3c(s2)COC[C@]32NCCc3ccc(Cl)c(F)c32)C[C@@H]1O] | 642 |
| 570 | 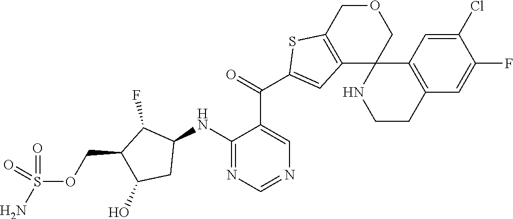 [SMILES: NS(=O)(=O)OC[C@H]1[C@H](F)[C@@H](Nc2ncncc2C(=O)c2cc3c(s2)COCC32NCCc3cc(F)c(Cl)cc32)C[C@@H]1O] | 642 |
| 571 | 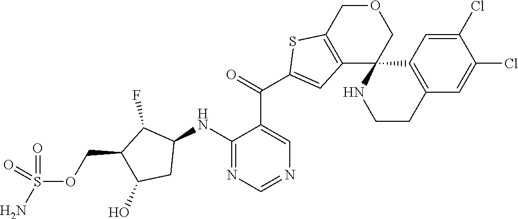 [SMILES: NS(=O)(=O)OC[C@H]1[C@H](F)[C@@H](Nc2ncncc2C(=O)c2cc3c(s2)COC[C@@]32NCCc3cc(Cl)c(Cl)cc32)C[C@@H]1O] | 642 |
| 572 | 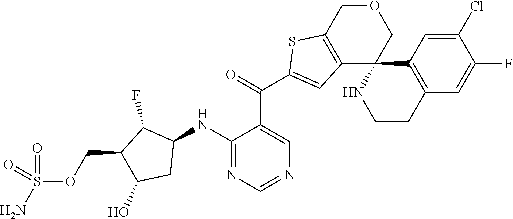 [SMILES: NS(=O)(=O)OC[C@H]1[C@H](F)[C@@H](Nc2ncncc2C(=O)c2cc3c(s2)COC[C@]32NCCc3cc(F)c(Cl)cc32)C[C@@H]1O] | 642 |
| 573 | 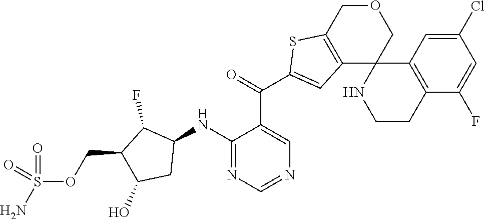 [SMILES: NS(=O)(=O)OC[C@H]1[C@H](F)[C@@H](Nc2ncncc2C(=O)c2cc3c(s2)COCC32NCCc3c(F)cc(Cl)cc32)C[C@@H]1O] | 642 |
| 574 | 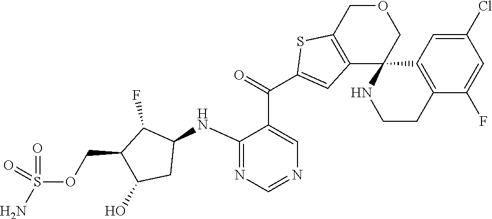 [SMILES: NS(=O)(=O)OC[C@H]1[C@H](F)[C@@H](Nc2ncncc2C(=O)c2cc3c(s2)COC[C@@]32NCCc3c(F)cc(Cl)cc32)C[C@@H]1O] | 642 |
| 575 | 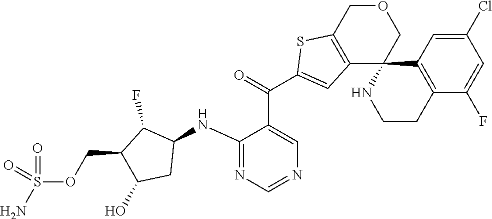 [SMILES: NS(=O)(=O)OC[C@H]1[C@H](F)[C@@H](Nc2ncncc2C(=O)c2cc3c(s2)COC[C@]32NCCc3c(F)cc(Cl)cc32)C[C@@H]1O] | 642 |
| 576 | 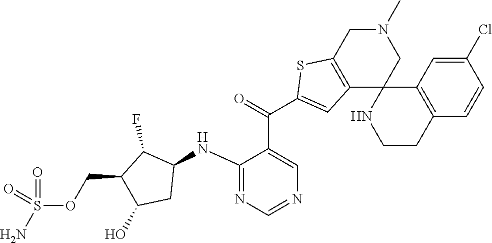 [SMILES: CN1Cc2sc(C(=O)c3cncnc3N[C@H]3C[C@H](O)[C@@H](COS(N)(=O)=O)[C@@H]3F)cc2C2(C1)NCCc1ccc(Cl)cc12] | 637 |
| 577 | 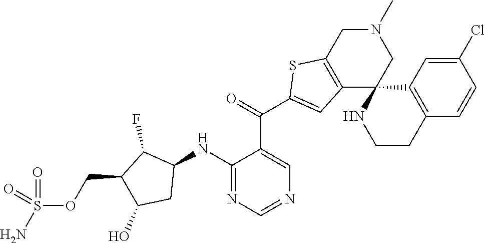 [SMILES: CN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12] | 637 |
| 578 | 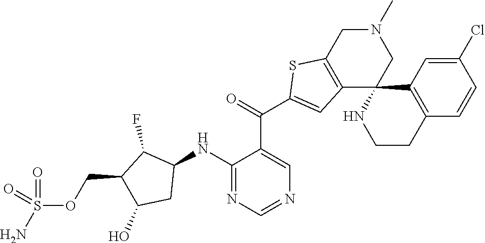 [SMILES: CN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12] | 637 |
| 579 | 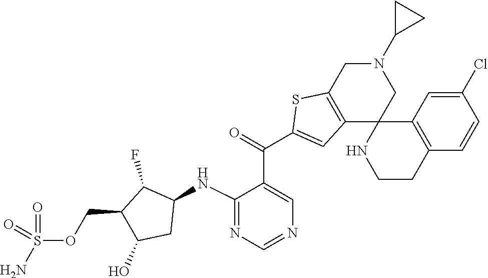 [SMILES: NS(=O)(=O)OC[C@H]1[C@H](F)[C@@H](Nc2ncncc2C(=O)c2cc3c(s2)CN(C2CC2)CC32NCCc3ccc(Cl)cc32)C[C@@H]1O] | 663 |
| 580 | 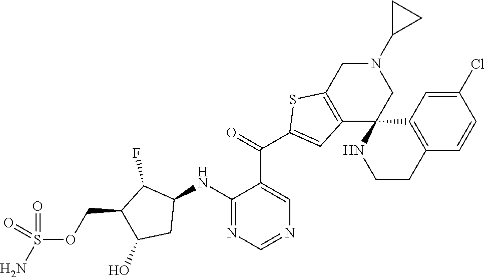 [SMILES: NS(=O)(=O)OC[C@H]1[C@H](F)[C@@H](Nc2ncncc2C(=O)c2cc3c(s2)CN(C2CC2)C[C@@]32NCCc3ccc(Cl)cc32)C[C@@H]1O] | 663 |
| 581 | 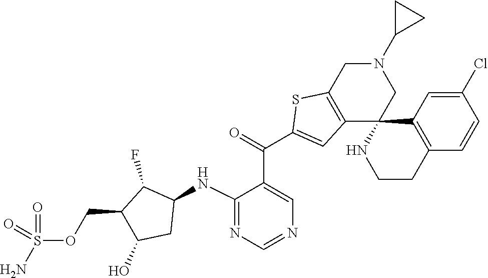 [SMILES: NS(=O)(=O)OC[C@H]1[C@H](F)[C@@H](Nc2ncncc2C(=O)c2cc3c(s2)CN(C2CC2)C[C@]32NCCc3ccc(Cl)cc32)C[C@@H]1O] | 663 |
| 582 | 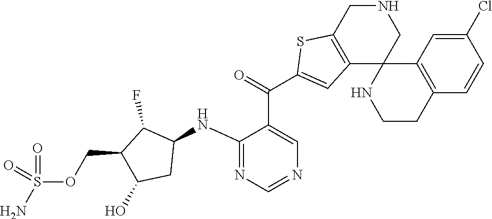 [SMILES: NS(=O)(=O)OC[C@H]1[C@H](F)[C@@H](Nc2ncncc2C(=O)c2cc3c(s2)CNCC32NCCc3ccc(Cl)cc32)C[C@@H]1O] | 623 |
| 583 | 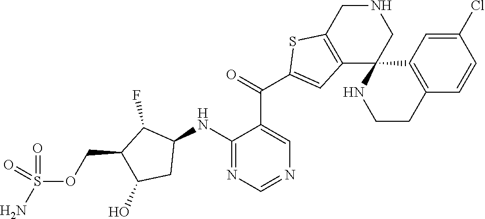 [SMILES: NS(=O)(=O)OC[C@H]1[C@H](F)[C@@H](Nc2ncncc2C(=O)c2cc3c(s2)CNC[C@@]32NCCc3ccc(Cl)cc32)C[C@@H]1O] | 623 |
| 584 | 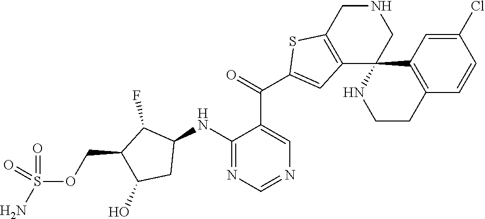 [SMILES: NS(=O)(=O)OC[C@H]1[C@H](F)[C@@H](Nc2ncncc2C(=O)c2cc3c(s2)CNC[C@]32NCCc3ccc(Cl)cc32)C[C@@H]1O] | 623 |
| 585 | 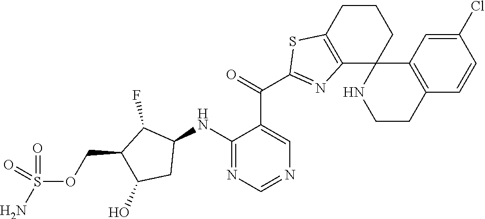 [SMILES: NS(=O)(=O)OC[C@H]1[C@H](F)[C@@H](Nc2ncncc2C(=O)c2nc3c(s2)CCCC32NCCc3ccc(Cl)cc32)C[C@@H]1O] | 623 |
| 586 | 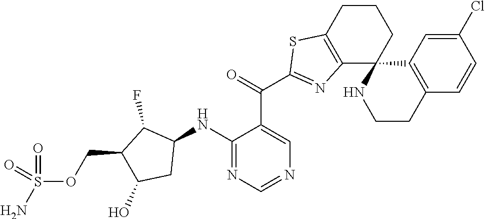 [SMILES: NS(=O)(=O)OC[C@H]1[C@H](F)[C@@H](Nc2ncncc2C(=O)c2nc3c(s2)CCC[C@@]32NCCc3ccc(Cl)cc32)C[C@@H]1O] | 623 |
| 587 | 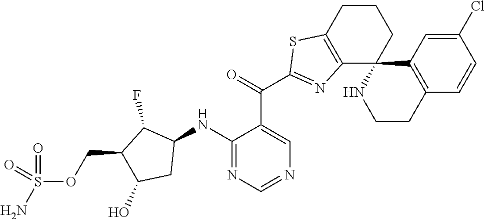 [SMILES: NS(=O)(=O)OC[C@H]1[C@H](F)[C@@H](Nc2ncncc2C(=O)c2nc3c(s2)CCC[C@]32NCCc3ccc(Cl)cc32)C[C@@H]1O] | 623 |
| 588 | 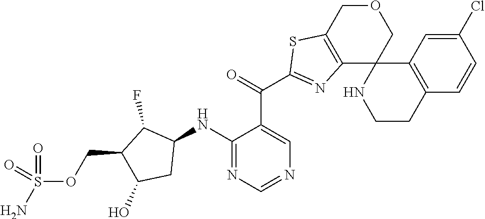 [SMILES: NS(=O)(=O)OC[C@H]1[C@H](F)[C@@H](Nc2ncncc2C(=O)c2nc3c(s2)COCC32NCCc3ccc(Cl)cc32)C[C@@H]1O] | 625 |
| 589 | 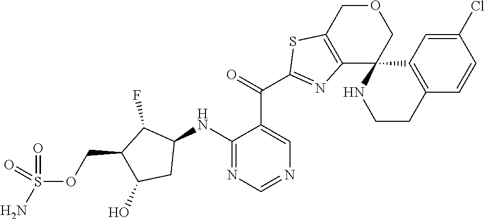 [SMILES: NS(=O)(=O)OC[C@H]1[C@H](F)[C@@H](Nc2ncncc2C(=O)c2nc3c(s2)COC[C@@]32NCCc3ccc(Cl)cc32)C[C@@H]1O] | 625 |
| 590 | 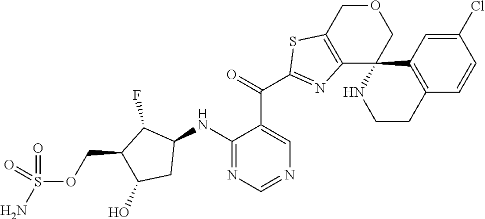 [SMILES: NS(=O)(=O)OC[C@H]1[C@H](F)[C@@H](Nc2ncncc2C(=O)c2nc3c(s2)COC[C@]32NCCc3ccc(Cl)cc32)C[C@@H]1O] | 625 |
| 591 | 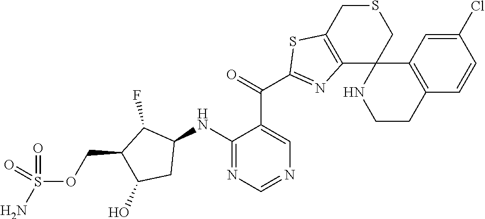 [SMILES: NS(=O)(=O)OC[C@H]1[C@H](F)[C@@H](Nc2ncncc2C(=O)c2nc3c(s2)CSCC32NCCc3ccc(Cl)cc32)C[C@@H]1O] | 641 |
| 592 | 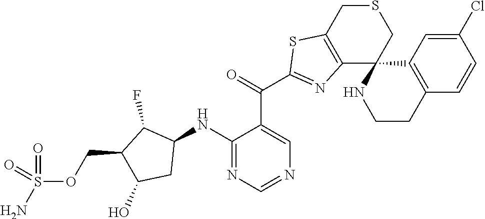 [SMILES: NS(=O)(=O)OC[C@H]1[C@H](F)[C@@H](Nc2ncncc2C(=O)c2nc3c(s2)CSC[C@@]32NCCc3ccc(Cl)cc32)C[C@@H]1O] | 641 |
| 593 | 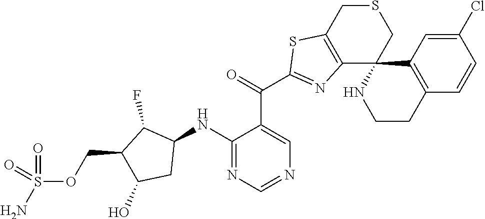 [SMILES: NS(=O)(=O)OC[C@H]1[C@H](F)[C@@H](Nc2ncncc2C(=O)c2nc3c(s2)CSC[C@]32NCCc3ccc(Cl)cc32)C[C@@H]1O] | 641 |
| 594 | 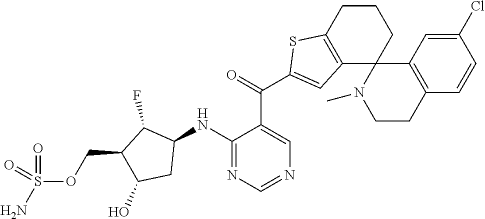 [SMILES: CN1CCc2ccc(Cl)cc2C12CCCc1sc(C(=O)c3cncnc3N[C@H]3C[C@H](O)[C@@H](COS(N)(=O)=O)[C@@H]3F)cc12] | 636 |
| 595 | 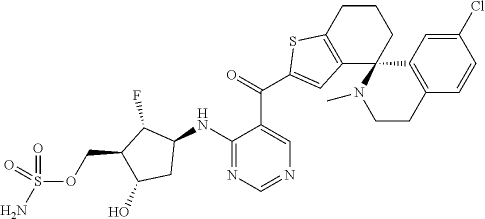 [SMILES: CN1CCc2ccc(Cl)cc2[C@]12CCCc1sc(C(=O)c3cncnc3N[C@H]3C[C@H](O)[C@@H](COS(N)(=O)=O)[C@@H]3F)cc12] | 636 |
| 596 | 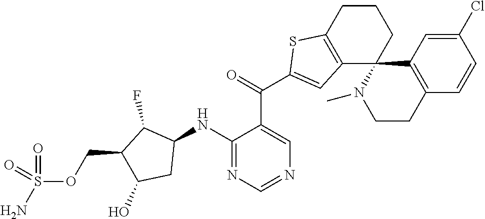 [SMILES: CN1CCc2ccc(Cl)cc2[C@@]12CCCc1sc(C(=O)c3cncnc3N[C@H]3C[C@H](O)[C@@H](COS(N)(=O)=O)[C@@H]3F)cc12] | 636 |
| 597 | 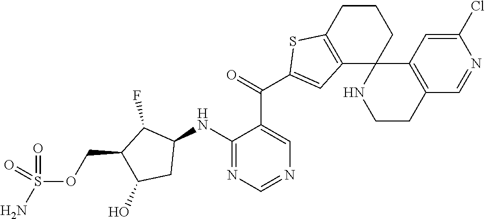 [SMILES: NS(=O)(=O)OC[C@H]1[C@H](F)[C@@H](Nc2ncncc2C(=O)c2cc3c(s2)CCCC32NCCc3cnc(Cl)cc32)C[C@@H]1O] | 623 |
| 598 | 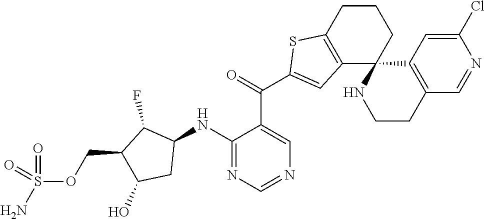 [SMILES: NS(=O)(=O)OC[C@H]1[C@H](F)[C@@H](Nc2ncncc2C(=O)c2cc3c(s2)CCC[C@@]32NCCc3cnc(Cl)cc32)C[C@@H]1O] | 623 |
| 599 | 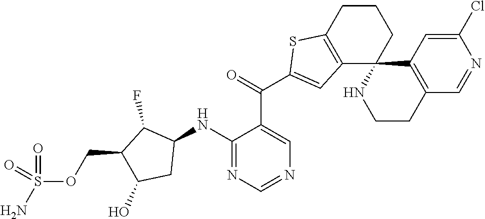 [SMILES: NS(=O)(=O)OC[C@H]1[C@H](F)[C@@H](Nc2ncncc2C(=O)c2cc3c(s2)CCC[C@]32NCCc3cnc(Cl)cc32)C[C@@H]1O] | 623 |
| 600 | 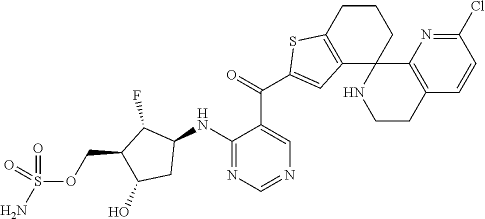 [SMILES: NS(=O)(=O)OC[C@H]1[C@H](F)[C@@H](Nc2ncncc2C(=O)c2cc3c(s2)CCCC32NCCc3ccc(Cl)nc32)C[C@@H]1O] | 623 |
| 601 | 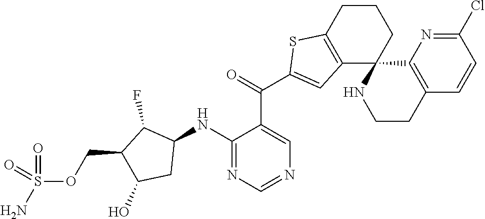 [SMILES: NS(=O)(=O)OC[C@H]1[C@H](F)[C@@H](Nc2ncncc2C(=O)c2cc3c(s2)CCC[C@@]32NCCc3ccc(Cl)nc32)C[C@@H]1O] | 623 |
| 602 | 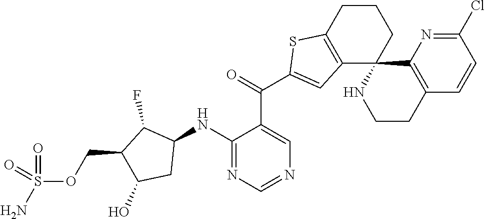 [SMILES: NS(=O)(=O)OC[C@H]1[C@H](F)[C@@H](Nc2ncncc2C(=O)c2cc3c(s2)CCC[C@]32NCCc3ccc(Cl)nc32)C[C@@H]1O] | 623 |
| 603 | 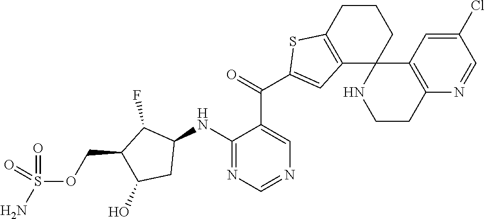 [SMILES: NS(=O)(=O)OC[C@H]1[C@H](F)[C@@H](Nc2ncncc2C(=O)c2cc3c(s2)CCCC32NCCc3ncc(Cl)cc32)C[C@@H]1O] | 623 |
| 604 | 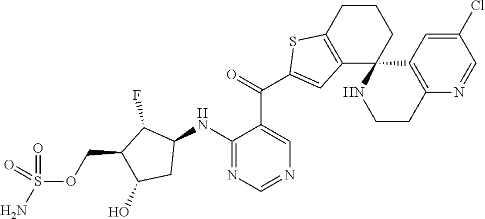 [SMILES: NS(=O)(=O)OC[C@H]1[C@H](F)[C@@H](Nc2ncncc2C(=O)c2cc3c(s2)CCC[C@@]32NCCc3ncc(Cl)cc32)C[C@@H]1O] | 623 |
| 605 | 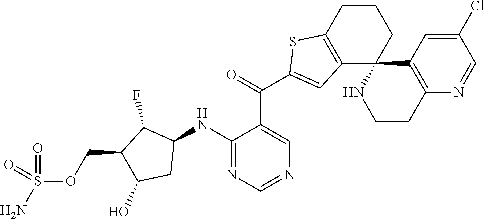 [SMILES: NS(=O)(=O)OC[C@H]1[C@H](F)[C@@H](Nc2ncncc2C(=O)c2cc3c(s2)CCC[C@]32NCCc3ncc(Cl)cc32)C[C@@H]1O] | 623 |
| 606 | 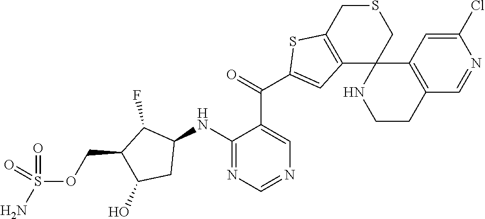 [SMILES: NS(=O)(=O)OC[C@H]1[C@H](F)[C@@H](Nc2ncncc2C(=O)c2cc3c(s2)CSCC32NCCc3cnc(Cl)cc32)C[C@@H]1O] | 641 |
| 607 | 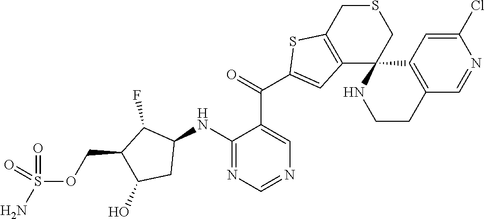 [SMILES: NS(=O)(=O)OC[C@H]1[C@H](F)[C@@H](Nc2ncncc2C(=O)c2cc3c(s2)CSC[C@@]32NCCc3cnc(Cl)cc32)C[C@@H]1O] | 641 |
| 608 | 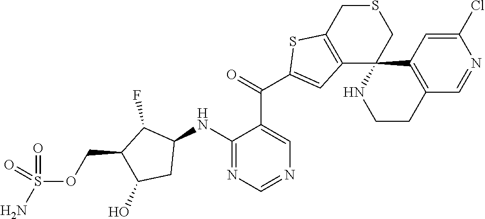 [SMILES: NS(=O)(=O)OC[C@H]1[C@H](F)[C@@H](Nc2ncncc2C(=O)c2cc3c(s2)CSC[C@]32NCCc3cnc(Cl)cc32)C[C@@H]1O] | 641 |
| 609 | 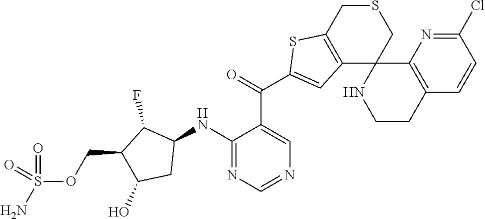 [SMILES: NS(=O)(=O)OC[C@H]1[C@H](F)[C@@H](Nc2ncncc2C(=O)c2cc3c(s2)CSCC32NCCc3ccc(Cl)nc32)C[C@@H]1O] | 641 |
| 610 | 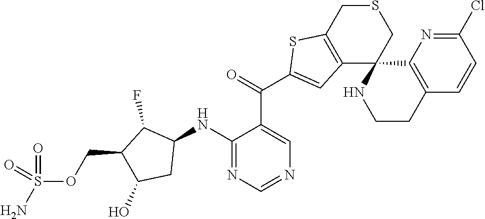 [SMILES: NS(=O)(=O)OC[C@H]1[C@H](F)[C@@H](Nc2ncncc2C(=O)c2cc3c(s2)CSC[C@@]32NCCc3ccc(Cl)nc32)C[C@@H]1O] | 641 |
| 611 | 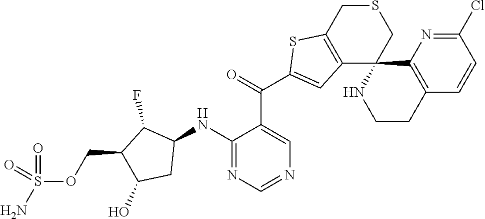 [SMILES: NS(=O)(=O)OC[C@H]1[C@H](F)[C@@H](Nc2ncncc2C(=O)c2cc3c(s2)CSC[C@]32NCCc3ccc(Cl)nc32)C[C@@H]1O] | 641 |
| 612 | 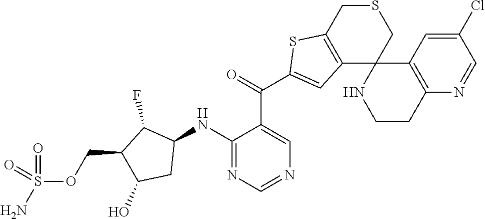 [SMILES: NS(=O)(=O)OC[C@H]1[C@H](F)[C@@H](Nc2ncncc2C(=O)c2cc3c(s2)CSCC32NCCc3ncc(Cl)cc32)C[C@@H]1O] | 641 |
| 613 | 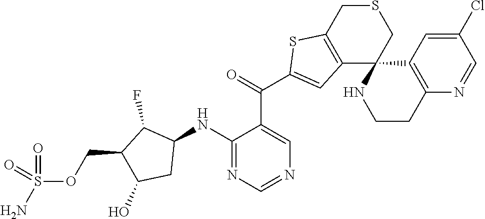 [SMILES: NS(=O)(=O)OC[C@H]1[C@H](F)[C@@H](Nc2ncncc2C(=O)c2cc3c(s2)CSC[C@@]32NCCc3ncc(Cl)cc32)C[C@@H]1O] | 641 |
| 614 | 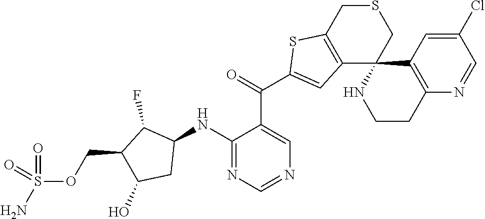 [SMILES: NS(=O)(=O)OC[C@H]1[C@H](F)[C@@H](Nc2ncncc2C(=O)c2cc3c(s2)CSC[C@]32NCCc3ncc(Cl)cc32)C[C@@H]1O] | 641 |
| 615 | 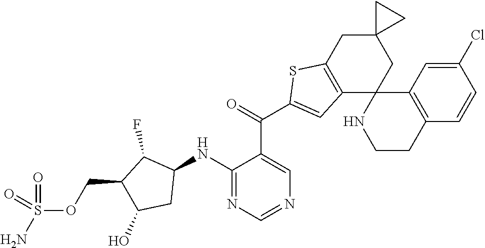 [SMILES: NS(=O)(=O)OC[C@H]1[C@H](F)[C@@H](Nc2ncncc2C(=O)c2cc3c(s2)CC2(CC2)CC32NCCc3ccc(Cl)cc32)C[C@@H]1O] | 648 |
| 616 | 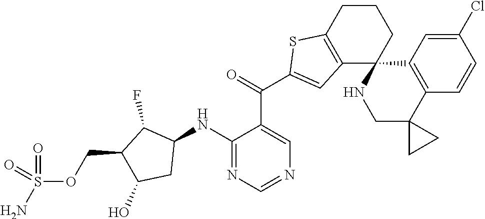 [SMILES: NS(=O)(=O)OC[C@H]1[C@H](F)[C@@H](Nc2ncncc2C(=O)c2cc3c(s2)CCC[C@@]32NCC3(CC3)c3ccc(Cl)cc32)C[C@@H]1O] | 648 |
| 617 | 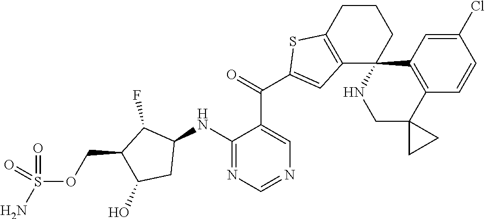 [SMILES: NS(=O)(=O)OC[C@H]1[C@H](F)[C@@H](Nc2ncncc2C(=O)c2cc3c(s2)CCC[C@]32NCC3(CC3)c3ccc(Cl)cc32)C[C@@H]1O] | 648 |
| 618 | 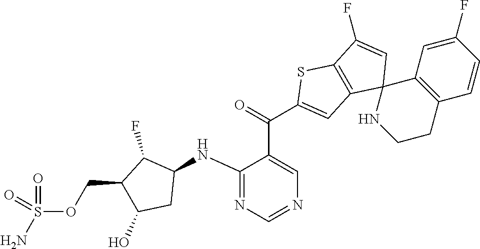 [SMILES: NS(=O)(=O)OC[C@H]1[C@H](F)[C@@H](Nc2ncncc2C(=O)c2cc3c(s2)C(F)=CC32NCCc3ccc(F)cc32)C[C@@H]1O] | 608 |
| 619 | 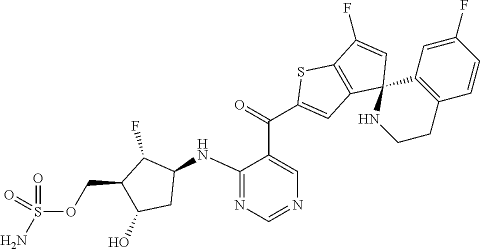 [SMILES: NS(=O)(=O)OC[C@H]1[C@H](F)[C@@H](Nc2ncncc2C(=O)c2cc3c(s2)C(F)=C[C@@]32NCCc3ccc(F)cc32)C[C@@H]1O] | 608 |
| 620 | 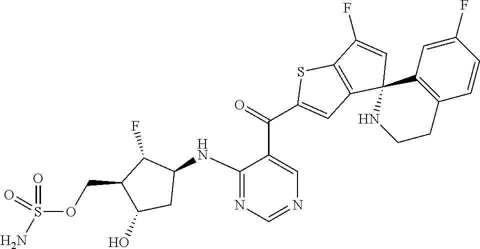 [SMILES: NS(=O)(=O)OC[C@H]1[C@H](F)[C@@H](Nc2ncncc2C(=O)c2cc3c(s2)C(F)=C[C@]32NCCc3ccc(F)cc32)C[C@@H]1O] | 608 |
| 621 | 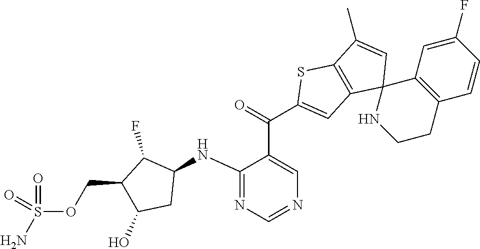 [SMILES: CC1=CC2(NCCc3ccc(F)cc32)c2cc(C(=O)c3cncnc3N[C@H]3C[C@H](O)[C@@H](COS(N)(=O)=O)[C@@H]3F)sc21] | 604 |
| 622 | 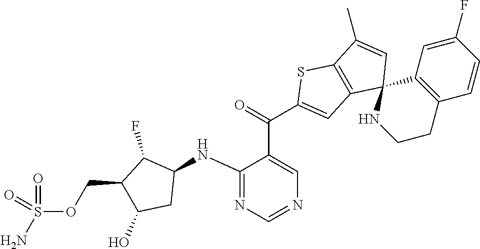 [SMILES: CC1=C[C@@]2(NCCc3ccc(F)cc32)c2cc(C(=O)c3cncnc3N[C@H]3C[C@H](O)[C@@H](COS(N)(=O)=O)[C@@H]3F)sc21] | 604 |
| 623 | 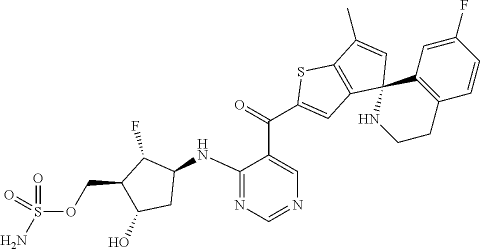 [SMILES: CC1=C[C@]2(NCCc3ccc(F)cc32)c2cc(C(=O)c3cncnc3N[C@H]3C[C@H](O)[C@@H](COS(N)(=O)=O)[C@@H]3F)sc21] | 604 |
| 624 | 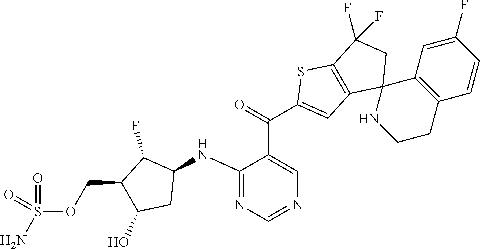 [SMILES: NS(=O)(=O)OC[C@H]1[C@H](F)[C@@H](Nc2ncncc2C(=O)c2cc3c(s2)C(F)(F)CC32NCCc3ccc(F)cc32)C[C@@H]1O] | 628 |
| 625 | 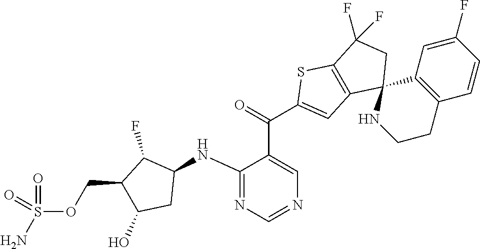 [SMILES: NS(=O)(=O)OC[C@H]1[C@H](F)[C@@H](Nc2ncncc2C(=O)c2cc3c(s2)C(F)(F)C[C@@]32NCCc3ccc(F)cc32)C[C@@H]1O] | 628 |
| 626 | 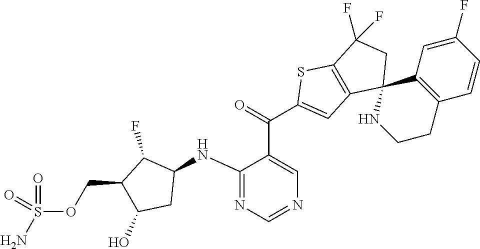 [SMILES: NS(=O)(=O)OC[C@H]1[C@H](F)[C@@H](Nc2ncncc2C(=O)c2cc3c(s2)C(F)(F)C[C@]32NCCc3ccc(F)cc32)C[C@@H]1O] | 628 |
| 627 | 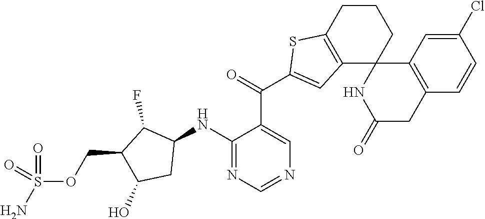 [SMILES: NS(=O)(=O)OC[C@H]1[C@H](F)[C@@H](Nc2ncncc2C(=O)c2cc3c(s2)CCCC32NC(=O)Cc3ccc(Cl)cc32)C[C@@H]1O] | 636 |
| 628 | 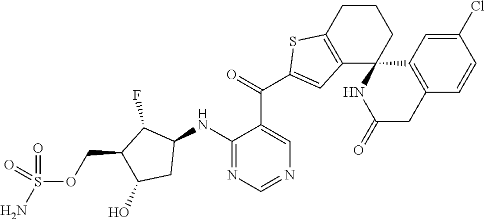 [SMILES: NS(=O)(=O)OC[C@H]1[C@H](F)[C@@H](Nc2ncncc2C(=O)c2cc3c(s2)CCC[C@@]32NC(=O)Cc3ccc(Cl)cc32)C[C@@H]1O] | 636 |
| 629 | 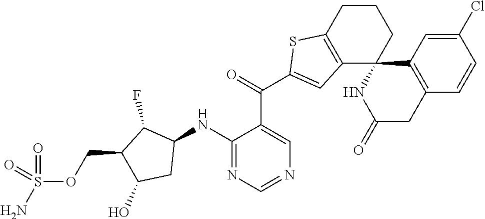 [SMILES: NS(=O)(=O)OC[C@H]1[C@H](F)[C@@H](Nc2ncncc2C(=O)c2cc3c(s2)CCC[C@]32NC(=O)Cc3ccc(Cl)cc32)C[C@@H]1O] | 636 |
| 630 | 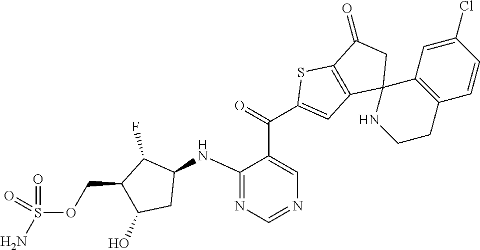 [SMILES: NS(=O)(=O)OC[C@H]1[C@H](F)[C@@H](Nc2ncncc2C(=O)c2cc3c(s2)C(=O)CC32NCCc3ccc(Cl)cc32)C[C@@H]1O] | 622 |
| 631 | 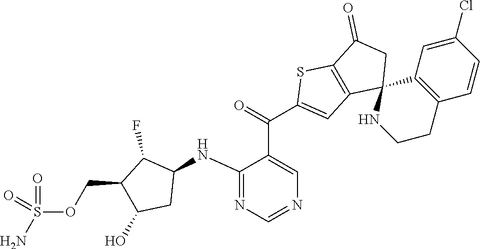 [SMILES: NS(=O)(=O)OC[C@H]1[C@H](F)[C@@H](Nc2ncncc2C(=O)c2cc3c(s2)C(=O)C[C@@]32NCCc3ccc(Cl)cc32)C[C@@H]1O] | 622 |
| 632 | 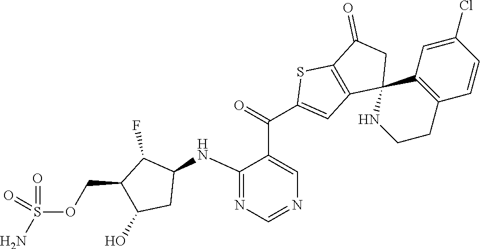 [SMILES: NS(=O)(=O)OC[C@H]1[C@H](F)[C@@H](Nc2ncncc2C(=O)c2cc3c(s2)C(=O)C[C@]32NCCc3ccc(Cl)cc32)C[C@@H]1O] | 622 |
| 633 | 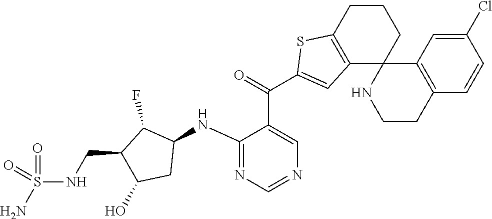 [SMILES: NS(=O)(=O)NC[C@H]1[C@H](F)[C@@H](Nc2ncncc2C(=O)c2cc3c(s2)CCCC32NCCc3ccc(Cl)cc32)C[C@@H]1O] | 621 |
| 634 | 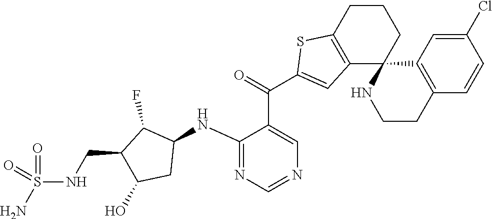 [SMILES: NS(=O)(=O)NC[C@H]1[C@H](F)[C@@H](Nc2ncncc2C(=O)c2cc3c(s2)CCC[C@@]32NCCc3ccc(Cl)cc32)C[C@@H]1O] | 621 |
| 635 | 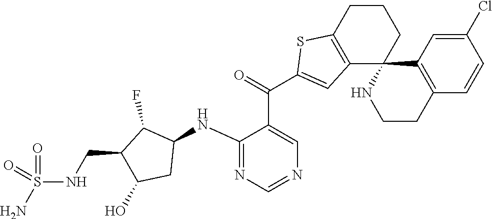 [SMILES: NS(=O)(=O)NC[C@H]1[C@H](F)[C@@H](Nc2ncncc2C(=O)c2cc3c(s2)CCC[C@]32NCCc3ccc(Cl)cc32)C[C@@H]1O] | 621 |
| 636 | 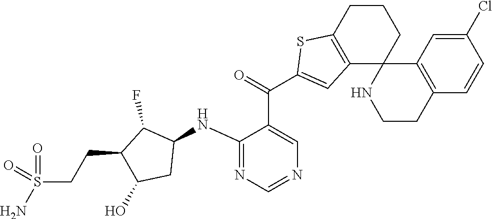 [SMILES: NS(=O)(=O)CC[C@H]1[C@H](F)[C@@H](Nc2ncncc2C(=O)c2cc3c(s2)CCCC32NCCc3ccc(Cl)cc32)C[C@@H]1O] | 620 |
| 637 | 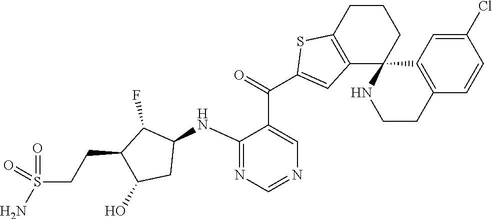 [SMILES: NS(=O)(=O)CC[C@H]1[C@H](F)[C@@H](Nc2ncncc2C(=O)c2cc3c(s2)CCC[C@@]32NCCc3ccc(Cl)cc32)C[C@@H]1O] | 620 |
| 638 | 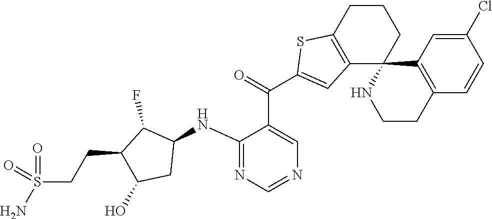 [SMILES: NS(=O)(=O)CC[C@H]1[C@H](F)[C@@H](Nc2ncncc2C(=O)c2cc3c(s2)CCC[C@]32NCCc3ccc(Cl)cc32)C[C@@H]1O] | 620 |
| 639 | 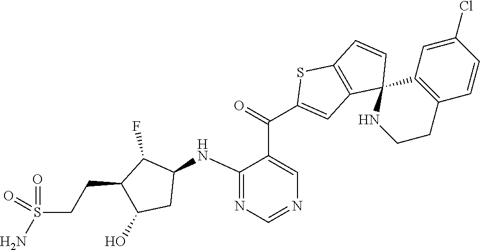 [SMILES: NS(=O)(=O)CC[C@H]1[C@H](F)[C@@H](Nc2ncncc2C(=O)c2cc3c(s2)C=C[C@@]32NCCc3ccc(Cl)cc32)C[C@@H]1O] | 604 |
| 640 | 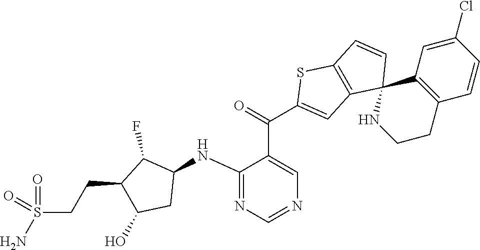 [SMILES: NS(=O)(=O)CC[C@H]1[C@H](F)[C@@H](Nc2ncncc2C(=O)c2cc3c(s2)C=C[C@]32NCCc3ccc(Cl)cc32)C[C@@H]1O] | 604 |
| 641 | 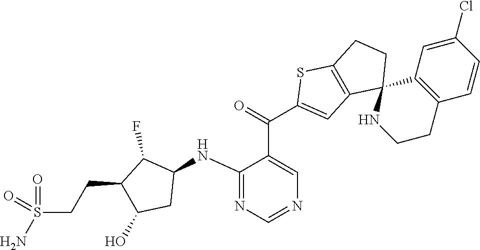 [SMILES: NS(=O)(=O)CC[C@H]1[C@H](F)[C@@H](Nc2ncncc2C(=O)c2cc3c(s2)CC[C@@]32NCCc3ccc(Cl)cc32)C[C@@H]1O] | 606 |
| 642 | 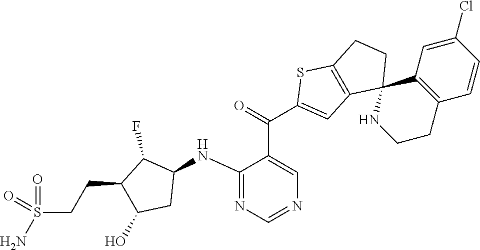 [SMILES: NS(=O)(=O)CC[C@H]1[C@H](F)[C@@H](Nc2ncncc2C(=O)c2cc3c(s2)CC[C@]32NCCc3ccc(Cl)cc32)C[C@@H]1O] | 606 |
| 643 | 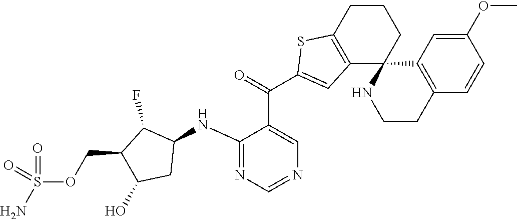 [SMILES: COc1ccc2c(c1)[C@]1(CCCc3sc(C(=O)c4cncnc4N[C@H]4C[C@H](O)[C@@H](COS(N)(=O)=O)[C@@H]4F)cc31)NCC2] | 618 |
| 644 | 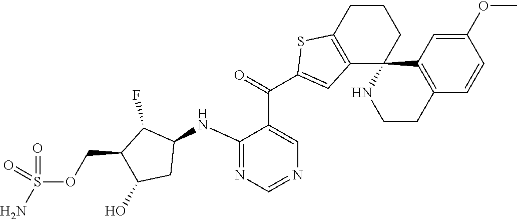 [SMILES: COc1ccc2c(c1)[C@@]1(CCCc3sc(C(=O)c4cncnc4N[C@H]4C[C@H](O)[C@@H](COS(N)(=O)=O)[C@@H]4F)cc31)NCC2] | 618 |
| 645 | 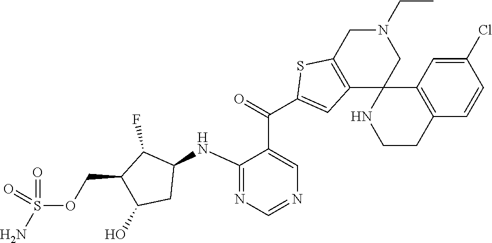 [SMILES: CCN1Cc2sc(C(=O)c3cncnc3N[C@H]3C[C@H](O)[C@@H](COS(N)(=O)=O)[C@@H]3F)cc2C2(C1)NCCc1ccc(Cl)cc12] | 651 |
| 646 | 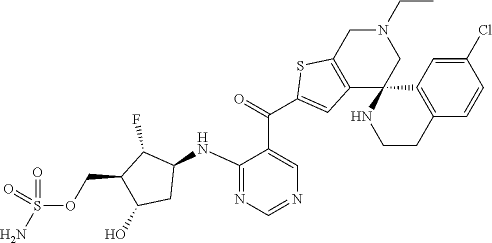 [SMILES: CCN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12] | 651 |
| 647 | 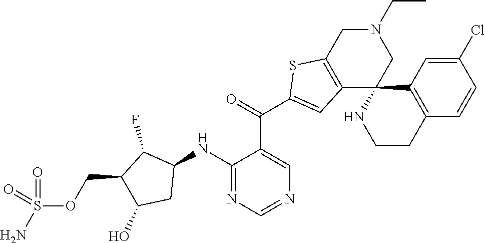 [SMILES: CCN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12] | 651 |
| 648 | 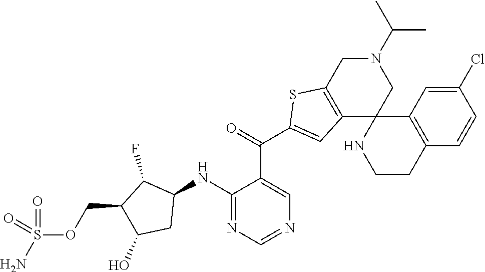 [SMILES: CC(C)N1Cc2sc(C(=O)c3cncnc3N[C@H]3C[C@H](O)[C@@H](COS(N)(=O)=O)[C@@H]3F)cc2C2(C1)NCCc1ccc(Cl)cc12] | 665 |
| 649 | 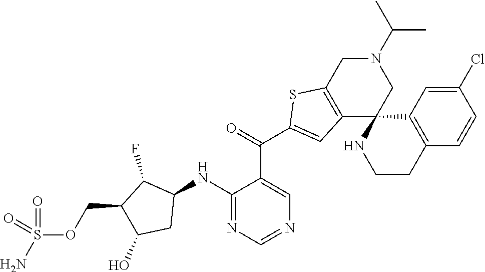 [SMILES: CC(C)N1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12] | 665 |
| 650 | 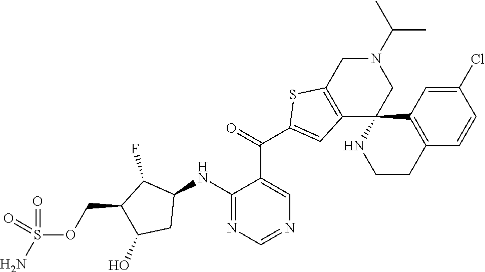 [SMILES: CC(C)N1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12] | 665 |
| 651 | 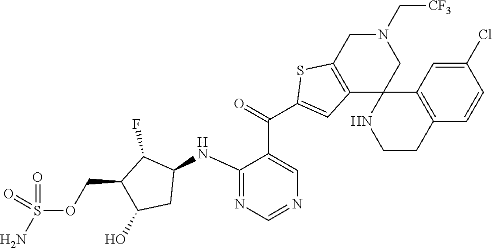 [SMILES: NS(=O)(=O)OC[C@H]1[C@H](F)[C@@H](Nc2ncncc2C(=O)c2cc3c(s2)CN(CC(F)(F)F)CC32NCCc3ccc(Cl)cc32)C[C@@H]1O] | 705 |
| 652 | 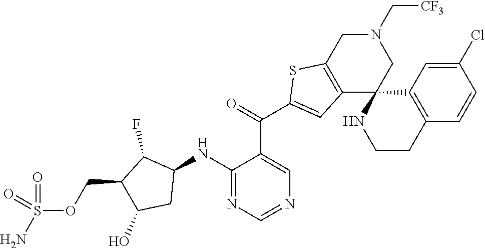 [SMILES: NS(=O)(=O)OC[C@H]1[C@H](F)[C@@H](Nc2ncncc2C(=O)c2cc3c(s2)CN(CC(F)(F)F)C[C@@]32NCCc3ccc(Cl)cc32)C[C@@H]1O] | 705 |
| 653 | 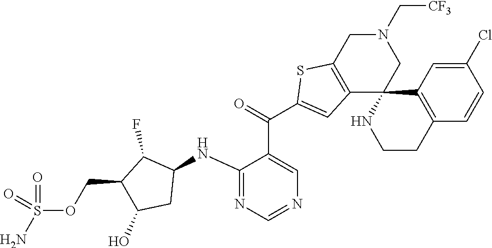 [SMILES: NS(=O)(=O)OC[C@H]1[C@H](F)[C@@H](Nc2ncncc2C(=O)c2cc3c(s2)CN(CC(F)(F)F)C[C@]32NCCc3ccc(Cl)cc32)C[C@@H]1O] | 705 |
| 654 | 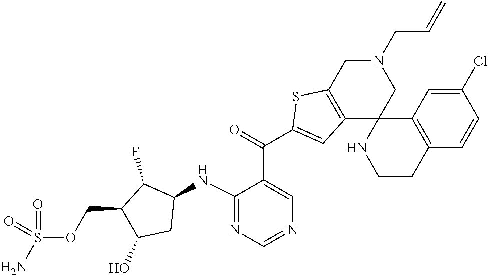 [SMILES: C=CCN1Cc2sc(C(=O)c3cncnc3N[C@H]3C[C@H](O)[C@@H](COS(N)(=O)=O)[C@@H]3F)cc2C2(C1)NCCc1ccc(Cl)cc12] | 663 |
| 655 | 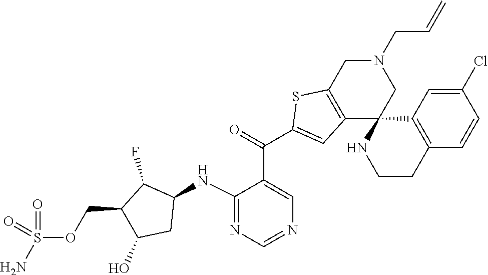 [SMILES: C=CCN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12] | 663 |
| 656 | 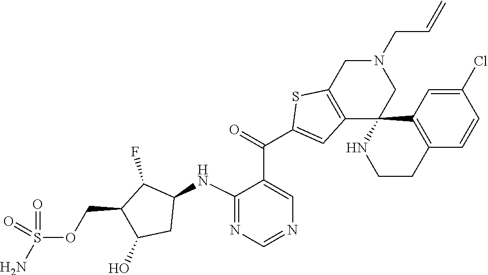 [SMILES: C=CCN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12] | 663 |
| 657 | 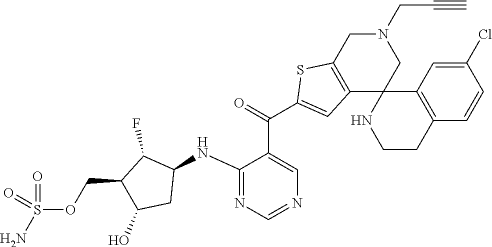 [SMILES: C#CCN1Cc2sc(C(=O)c3cncnc3N[C@H]3C[C@H](O)[C@@H](COS(N)(=O)=O)[C@@H]3F)cc2C2(C1)NCCc1ccc(Cl)cc12] | 661 |
| 658 | 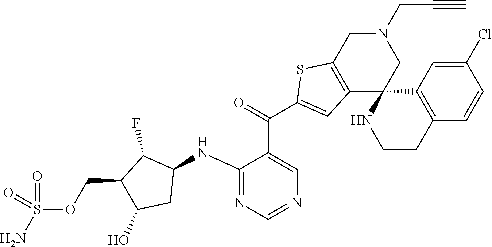 [SMILES: C#CCN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12] | 661 |
| 659 | 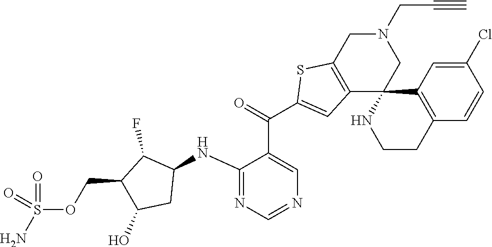 [SMILES: C#CCN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12] | 661 |
| 660 | 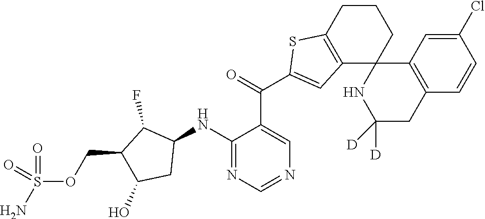 [SMILES: [2H]C1([2H])Cc2ccc(Cl)cc2C2(CCCc3sc(C(=O)c4cncnc4N[C@H]4C[C@H](O)[C@@H](COS(N)(=O)=O)[C@@H]4F)cc32)N1] | 624 |
| 661 | 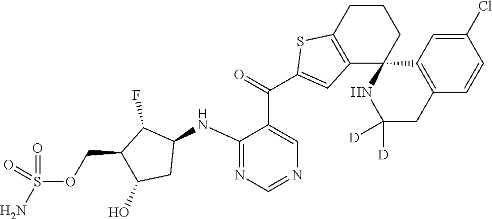 [SMILES: [2H]C1([2H])Cc2ccc(Cl)cc2[C@]2(CCCc3sc(C(=O)c4cncnc4N[C@H]4C[C@H](O)[C@@H](COS(N)(=O)=O)[C@@H]4F)cc32)N1] | 624 |
| 662 | 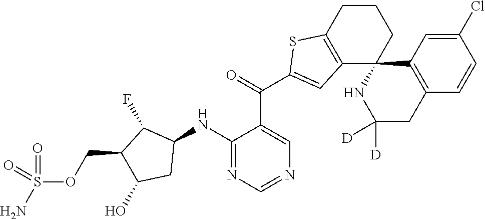 [SMILES: [2H]C1([2H])Cc2ccc(Cl)cc2[C@@]2(CCCc3sc(C(=O)c4cncnc4N[C@H]4C[C@H](O)[C@@H](COS(N)(=O)=O)[C@@H]4F)cc32)N1] | 624 |
| 663 | 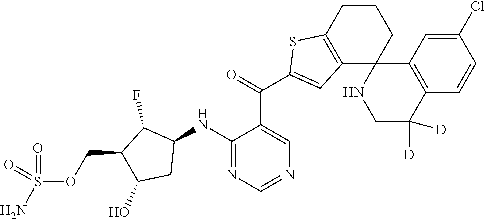 [SMILES: [2H]C1([2H])CNC2(CCCc3sc(C(=O)c4cncnc4N[C@H]4C[C@H](O)[C@@H](COS(N)(=O)=O)[C@@H]4F)cc32)c2cc(Cl)ccc21] | 624 |
| 664 | 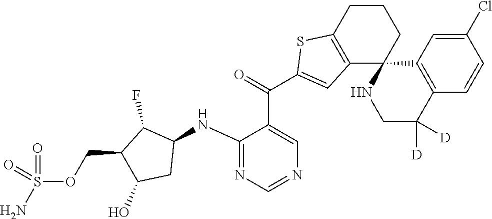 [SMILES: [2H]C1([2H])CN[C@@]2(CCCc3sc(C(=O)c4cncnc4N[C@H]4C[C@H](O)[C@@H](COS(N)(=O)=O)[C@@H]4F)cc32)c2cc(Cl)ccc21] | 624 |
| 665 | 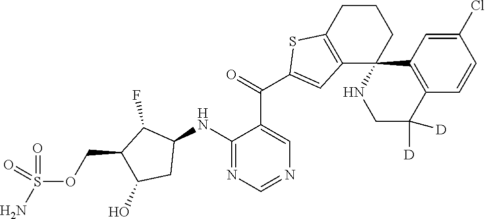 [SMILES: [2H]C1([2H])CN[C@]2(CCCc3sc(C(=O)c4cncnc4N[C@H]4C[C@H](O)[C@@H](COS(N)(=O)=O)[C@@H]4F)cc32)c2cc(Cl)ccc21] | 624 |
| 666 | 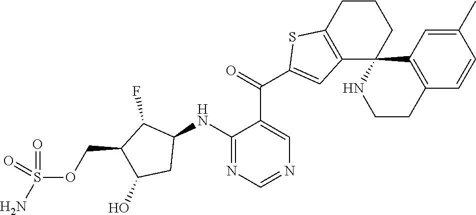 [SMILES: Cc1ccc2c(c1)[C@@]1(CCCc3sc(C(=O)c4cncnc4N[C@H]4C[C@H](O)[C@@H](COS(N)(=O)=O)[C@@H]4F)cc31)NCC2] | 602 |
| 667 | 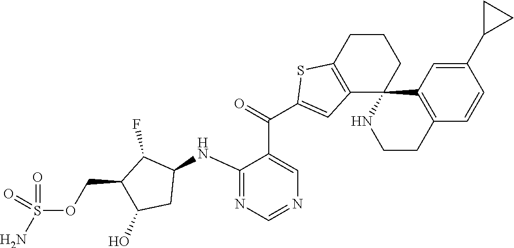 [SMILES: NS(=O)(=O)OC[C@H]1[C@H](F)[C@@H](Nc2ncncc2C(=O)c2cc3c(s2)CCC[C@]32NCCc3ccc(C4CC4)cc32)C[C@@H]1O] | 628 |
| 668 | 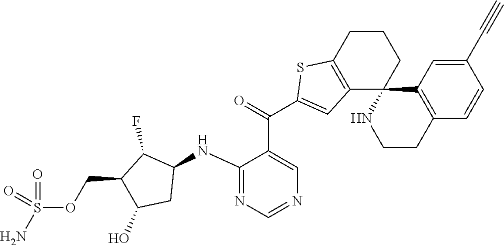 [SMILES: C#Cc1ccc2c(c1)[C@@]1(CCCc3sc(C(=O)c4cncnc4N[C@H]4C[C@H](O)[C@@H](COS(N)(=O)=O)[C@@H]4F)cc31)NCC2] | 612 |
| 669 | 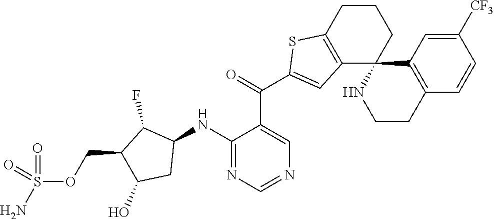 [SMILES: NS(=O)(=O)OC[C@H]1[C@H](F)[C@@H](Nc2ncncc2C(=O)c2cc3c(s2)CCC[C@]32NCCc3ccc(C(F)(F)F)cc32)C[C@@H]1O] | 656 |
| 670 | 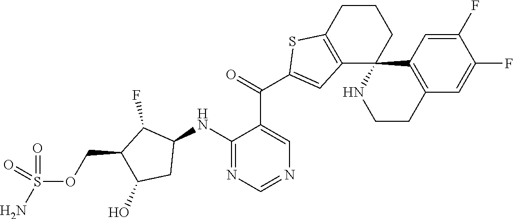 [SMILES: NS(=O)(=O)OC[C@H]1[C@H](F)[C@@H](Nc2ncncc2C(=O)c2cc3c(s2)CCC[C@]32NCCc3cc(F)c(F)cc32)C[C@@H]1O] | 624 |
| 671 | 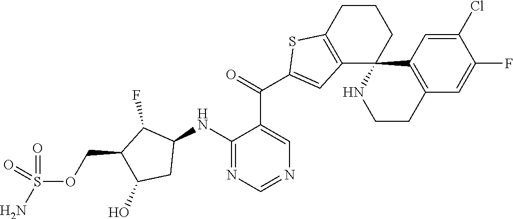 [SMILES: NS(=O)(=O)OC[C@H]1[C@H](F)[C@@H](Nc2ncncc2C(=O)c2cc3c(s2)CCC[C@]32NCCc3cc(F)c(Cl)cc32)C[C@@H]1O] | 640 |
| 672 | 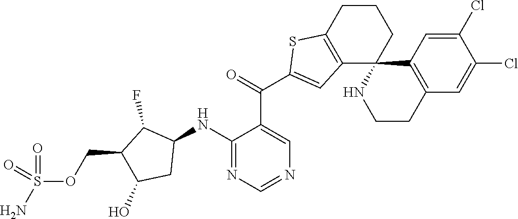 [SMILES: NS(=O)(=O)OC[C@H]1[C@H](F)[C@@H](Nc2ncncc2C(=O)c2cc3c(s2)CCC[C@]32NCCc3cc(Cl)c(Cl)cc32)C[C@@H]1O] | 656 |
| 673 | 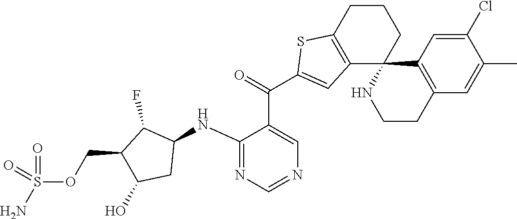 [SMILES: Cc1cc2c(cc1Cl)[C@@]1(CCCc3sc(C(=O)c4cncnc4N[C@H]4C[C@H](O)[C@@H](COS(N)(=O)=O)[C@@H]4F)cc31)NCC2] | 636 |
| 674 | 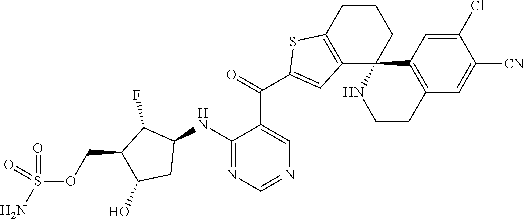 [SMILES: N#Cc1cc2c(cc1Cl)[C@@]1(CCCc3sc(C(=O)c4cncnc4N[C@H]4C[C@H](O)[C@@H](COS(N)(=O)=O)[C@@H]4F)cc31)NCC2] | 647 |
| 675 | 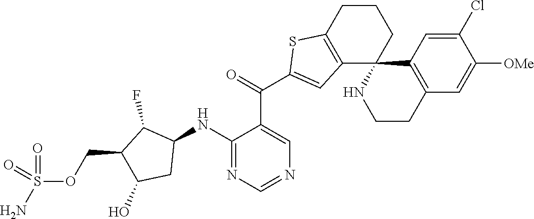 [SMILES: COc1cc2c(cc1Cl)[C@@]1(CCCc3sc(C(=O)c4cncnc4N[C@H]4C[C@H](O)[C@@H](COS(N)(=O)=O)[C@@H]4F)cc31)NCC2] | 652 |
| 676 | 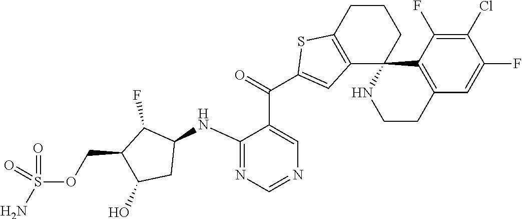 [SMILES: NS(=O)(=O)OC[C@H]1[C@H](F)[C@@H](Nc2ncncc2C(=O)c2cc3c(s2)CCC[C@]32NCCc3cc(F)c(Cl)c(F)c32)C[C@@H]1O] | 658 |
| 677 | 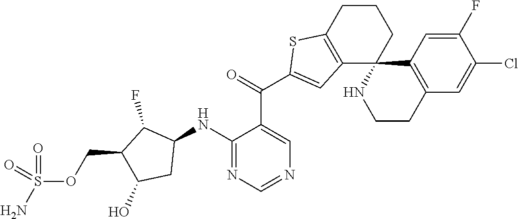 [SMILES: NS(=O)(=O)OC[C@H]1[C@H](F)[C@@H](Nc2ncncc2C(=O)c2cc3c(s2)CCC[C@]32NCCc3cc(Cl)c(F)cc32)C[C@@H]1O] | 640 |
| 678 | 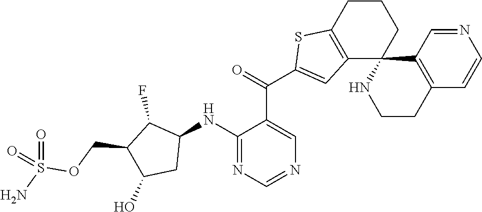 [SMILES: NS(=O)(=O)OC[C@H]1[C@H](F)[C@@H](Nc2ncncc2C(=O)c2cc3c(s2)CCC[C@]32NCCc3ccncc32)C[C@@H]1O] | 589 |
| 679 | 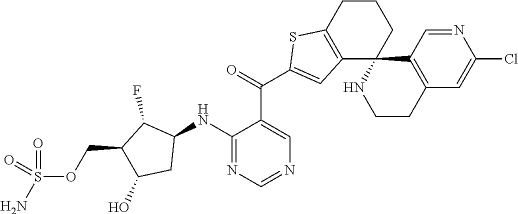 [SMILES: NS(=O)(=O)OC[C@H]1[C@H](F)[C@@H](Nc2ncncc2C(=O)c2cc3c(s2)CCC[C@]32NCCc3cc(Cl)ncc32)C[C@@H]1O] | 623 |
| 680 | 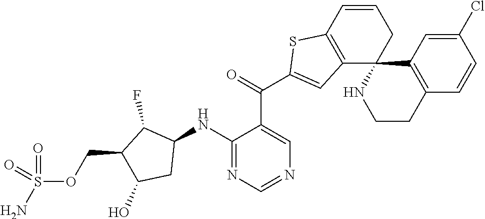 [SMILES: NS(=O)(=O)OC[C@H]1[C@H](F)[C@@H](Nc2ncncc2C(=O)c2cc3c(s2)C=CC[C@]32NCCc3ccc(Cl)cc32)C[C@@H]1O] | 620 |
| 681 | 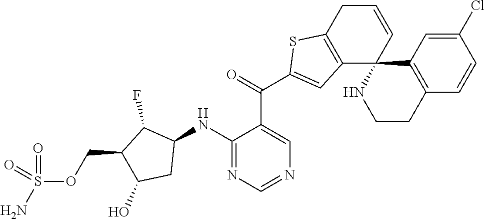 [SMILES: NS(=O)(=O)OC[C@H]1[C@H](F)[C@@H](Nc2ncncc2C(=O)c2cc3c(s2)CC=C[C@]32NCCc3ccc(Cl)cc32)C[C@@H]1O] | 620 |
| 682 | 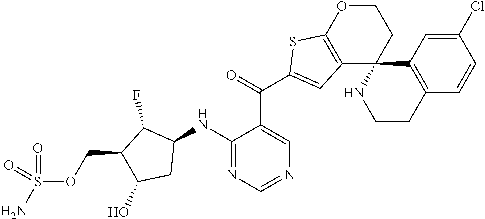 [SMILES: NS(=O)(=O)OC[C@H]1[C@H](F)[C@@H](Nc2ncncc2C(=O)c2cc3c(s2)OCC[C@]32NCCc3ccc(Cl)cc32)C[C@@H]1O] | 624 |
| 683 | 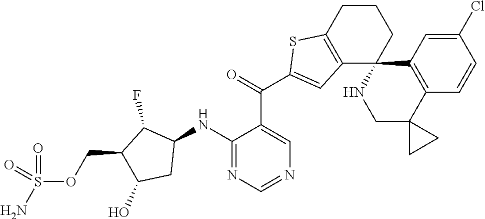 [SMILES: NS(=O)(=O)OC[C@H]1[C@H](F)[C@@H](Nc2ncncc2C(=O)c2cc3c(s2)CCC[C@]32NCC3(CC3)c3ccc(Cl)cc32)C[C@@H]1O] | 648 |
| 684 | 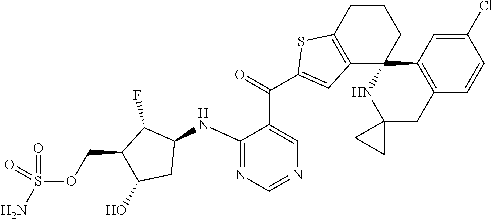 | 648 |
| 685 | 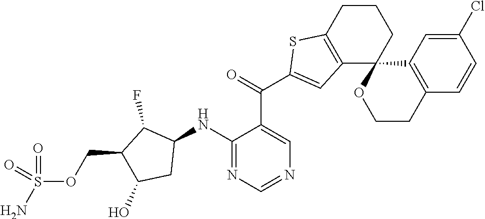 [SMILES: NS(=O)(=O)OC[C@H]1[C@H](F)[C@@H](Nc2ncncc2C(=O)c2cc3c(s2)CCC[C@@]32OCCc3ccc(Cl)cc32)C[C@@H]1O] | 623 |
| 686 | 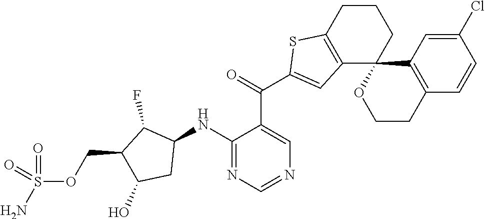 [SMILES: NS(=O)(=O)OC[C@H]1[C@H](F)[C@@H](Nc2ncncc2C(=O)c2cc3c(s2)CCC[C@]32OCCc3ccc(Cl)cc32)C[C@@H]1O] | 623 |
| 687 | 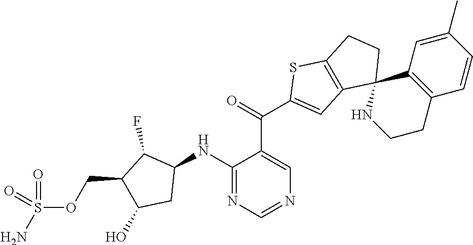 [SMILES: Cc1ccc2c(c1)[C@@]1(CCc3sc(C(=O)c4cncnc4N[C@H]4C[C@H](O)[C@@H](COS(N)(=O)=O)[C@@H]4F)cc31)NCC2] | 588 |
| 688 | 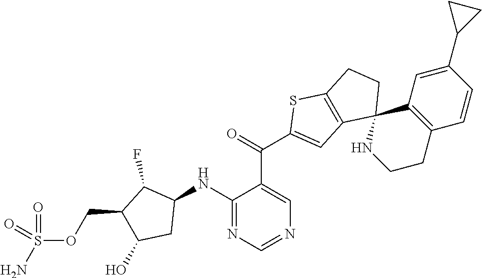 [SMILES: NS(=O)(=O)OC[C@H]1[C@H](F)[C@@H](Nc2ncncc2C(=O)c2cc3c(s2)CC[C@]32NCCc3ccc(C4CC4)cc32)C[C@@H]1O] | 614 |
| 689 | 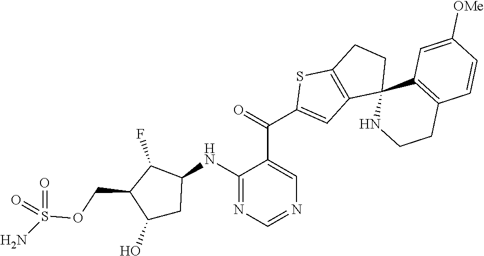 [SMILES: COc1ccc2c(c1)[C@@]1(CCc3sc(C(=O)c4cncnc4N[C@H]4C[C@H](O)[C@@H](COS(N)(=O)=O)[C@@H]4F)cc31)NCC2] | 604 |
| 690 | 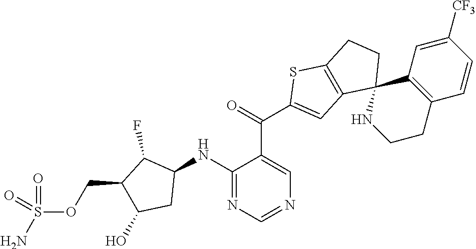 [SMILES: NS(=O)(=O)OC[C@H]1[C@H](F)[C@@H](Nc2ncncc2C(=O)c2cc3c(s2)CC[C@]32NCCc3ccc(C(F)(F)F)cc32)C[C@@H]1O] | 642 |
| 691 | 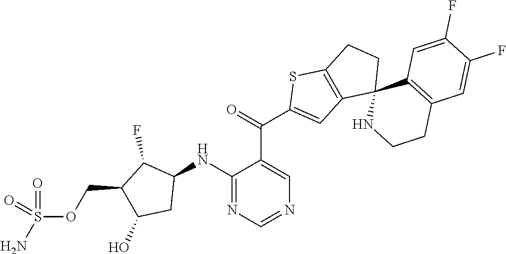 [SMILES: NS(=O)(=O)OC[C@H]1[C@H](F)[C@@H](Nc2ncncc2C(=O)c2cc3c(s2)CC[C@]32NCCc3cc(F)c(F)cc32)C[C@@H]1O] | 610 |
| 692 | 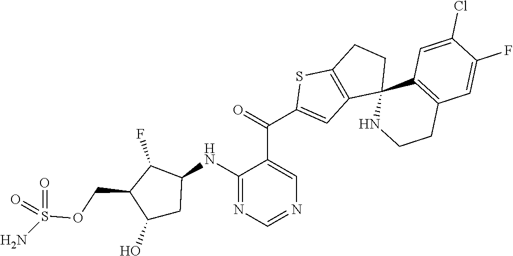 [SMILES: NS(=O)(=O)OC[C@H]1[C@H](F)[C@@H](Nc2ncncc2C(=O)c2cc3c(s2)CC[C@]32NCCc3cc(F)c(Cl)cc32)C[C@@H]1O] | 626 |
| 693 | 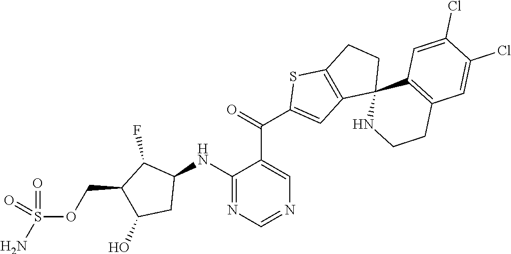 [SMILES: NS(=O)(=O)OC[C@H]1[C@H](F)[C@@H](Nc2ncncc2C(=O)c2cc3c(s2)CC[C@]32NCCc3cc(Cl)c(Cl)cc32)C[C@@H]1O] | 642 |
| 694 | 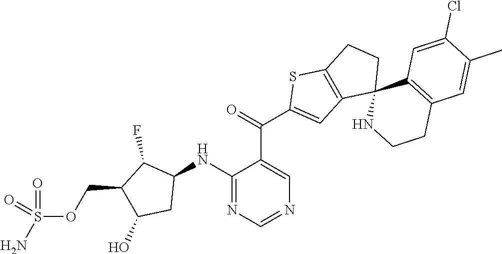 [SMILES: Cc1cc2c(cc1Cl)[C@@]1(CCc3sc(C(=O)c4cncnc4N[C@H]4C[C@H](O)[C@@H](COS(N)(=O)=O)[C@@H]4F)cc31)NCC2] | 622 |
| 695 | 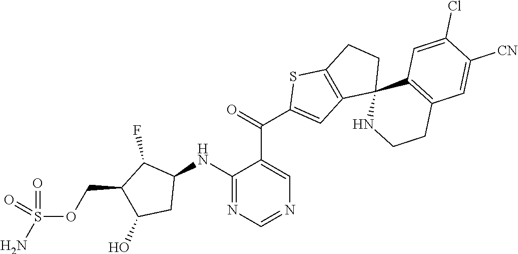 [SMILES: N#Cc1cc2c(cc1Cl)[C@@]1(CCc3sc(C(=O)c4cncnc4N[C@H]4C[C@H](O)[C@@H](COS(N)(=O)=O)[C@@H]4F)cc31)NCC2] | 633 |
| 696 | 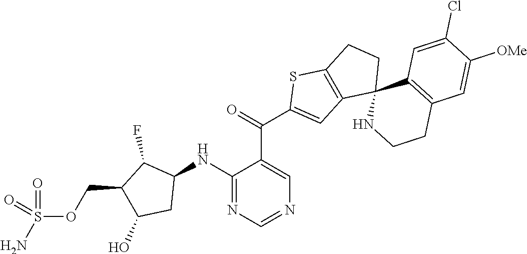 [SMILES: COc1cc2c(cc1Cl)[C@@]1(CCc3sc(C(=O)c4cncnc4N[C@H]4C[C@H](O)[C@@H](COS(N)(=O)=O)[C@@H]4F)cc31)NCC2] | 638 |
| 697 | 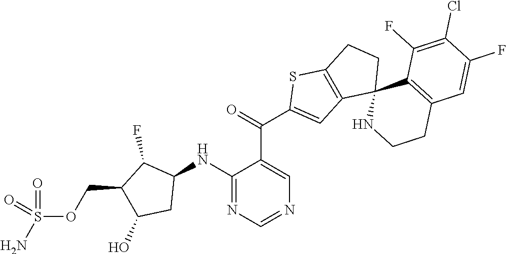 [SMILES: NS(=O)(=O)OC[C@H]1[C@H](F)[C@@H](Nc2ncncc2C(=O)c2cc3c(s2)CC[C@]32NCCc3cc(F)c(Cl)c(F)c32)C[C@@H]1O] | 644 |
| 698 | 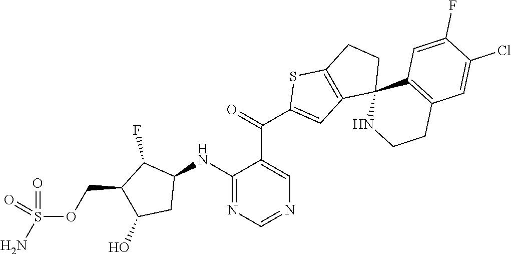 [SMILES: NS(=O)(=O)OC[C@H]1[C@H](F)[C@@H](Nc2ncncc2C(=O)c2cc3c(s2)CC[C@]32NCCc3cc(Cl)c(F)cc32)C[C@@H]1O] | 626 |
| 699 | 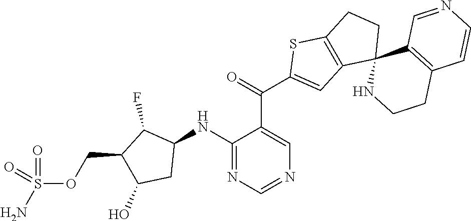 [SMILES: NS(=O)(=O)OC[C@H]1[C@H](F)[C@@H](Nc2ncncc2C(=O)c2cc3c(s2)CC[C@]32NCCc3ccncc32)C[C@@H]1O] | 575 |
| 700 | 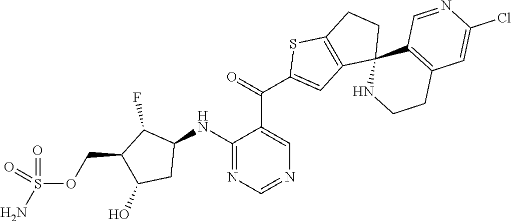 [SMILES: NS(=O)(=O)OC[C@H]1[C@H](F)[C@@H](Nc2ncncc2C(=O)c2cc3c(s2)CC[C@]32NCCc3cc(Cl)ncc32)C[C@@H]1O] | 609 |
| 701 | 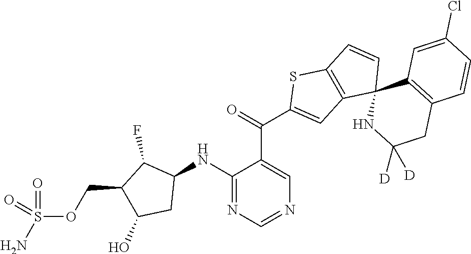 [SMILES: [2H]C1([2H])Cc2ccc(Cl)cc2[C@@]2(C=Cc3sc(C(=O)c4cncnc4N[C@H]4C[C@H](O)[C@@H](COS(N)(=O)=O)[C@@H]4F)cc32)N1] | 608 |
| 702 | 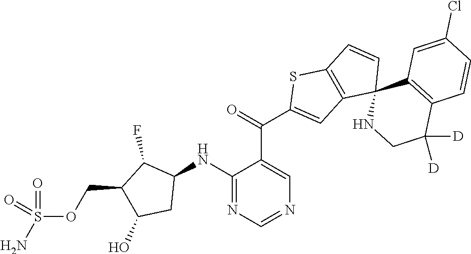 [SMILES: [2H]C1([2H])CN[C@]2(C=Cc3sc(C(=O)c4cncnc4N[C@H]4C[C@H](O)[C@@H](COS(N)(=O)=O)[C@@H]4F)cc32)c2cc(Cl)ccc21] | 608 |
| 703 | 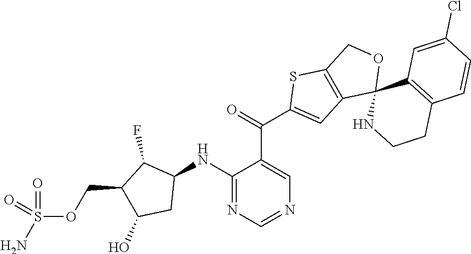 [SMILES: NS(=O)(=O)OC[C@H]1[C@H](F)[C@@H](Nc2ncncc2C(=O)c2cc3c(s2)CO[C@]32NCCc3ccc(Cl)cc32)C[C@@H]1O] | 610 |
| 704 | 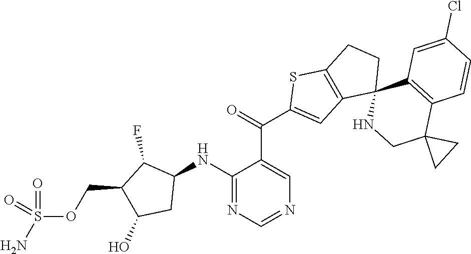 [SMILES: NS(=O)(=O)OC[C@H]1[C@H](F)[C@@H](Nc2ncncc2C(=O)c2cc3c(s2)CC[C@]32NCC3(CC3)c3ccc(Cl)cc32)C[C@@H]1O] | 634 |
| 705 | 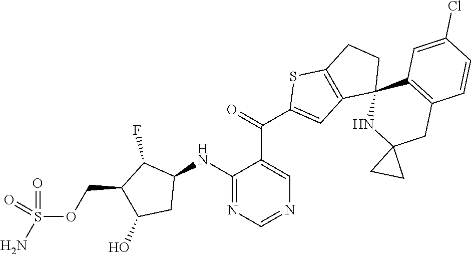 [SMILES: NS(=O)(=O)OC[C@H]1[C@H](F)[C@@H](Nc2ncncc2C(=O)c2cc3c(s2)CC[C@]32NC3(CC3)Cc3ccc(Cl)cc32)C[C@@H]1O] | 634 |
| 706 | 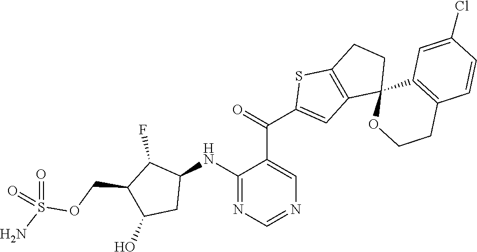 [SMILES: NS(=O)(=O)OC[C@H]1[C@H](F)[C@@H](Nc2ncncc2C(=O)c2cc3c(s2)CC[C@@]32OCCc3ccc(Cl)cc32)C[C@@H]1O] | 609 |
| 707 | 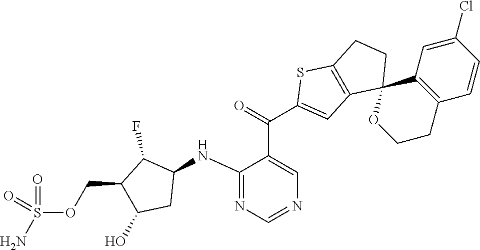 [SMILES: NS(=O)(=O)OC[C@H]1[C@H](F)[C@@H](Nc2ncncc2C(=O)c2cc3c(s2)CC[C@]32OCCc3ccc(Cl)cc32)C[C@@H]1O] | 609 |
| 708 | 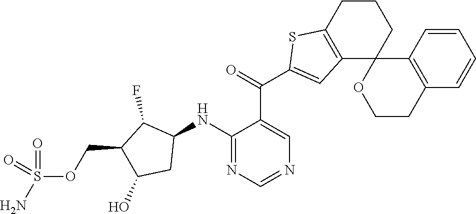 [SMILES: NS(=O)(=O)OC[C@H]1[C@H](F)[C@@H](Nc2ncncc2C(=O)c2cc3c(s2)CCCC32OCCc3ccccc32)C[C@@H]1O] | 589 |
| 709 | 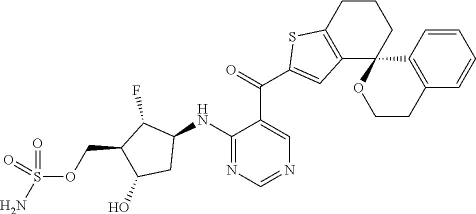 [SMILES: NS(=O)(=O)OC[C@H]1[C@H](F)[C@@H](Nc2ncncc2C(=O)c2cc3c(s2)CCC[C@@]32OCCc3ccccc32)C[C@@H]1O] | 589 |
| 710 | 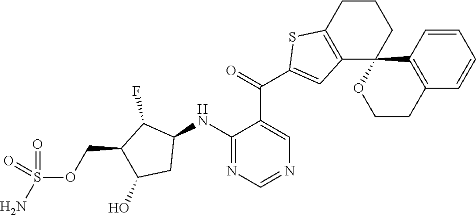 [SMILES: NS(=O)(=O)OC[C@H]1[C@H](F)[C@@H](Nc2ncncc2C(=O)c2cc3c(s2)CCC[C@]32OCCc3ccccc32)C[C@@H]1O] | 589 |
| 711 | 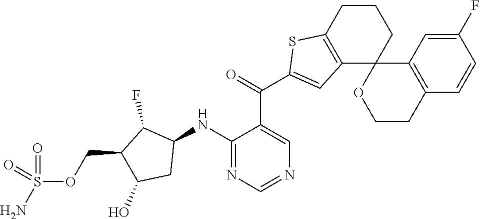 [SMILES: NS(=O)(=O)OC[C@H]1[C@H](F)[C@@H](Nc2ncncc2C(=O)c2cc3c(s2)CCCC32OCCc3ccc(F)cc32)C[C@@H]1O] | 607 |
| 712 | 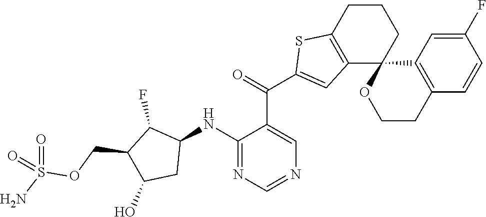 [SMILES: NS(=O)(=O)OC[C@H]1[C@H](F)[C@@H](Nc2ncncc2C(=O)c2cc3c(s2)CCC[C@@]32OCCc3ccc(F)cc32)C[C@@H]1O] | 607 |
| 713 | 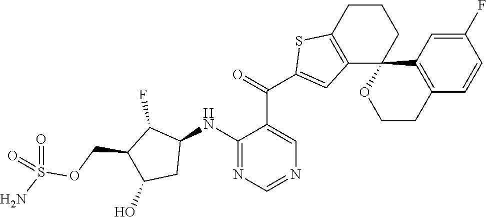 [SMILES: NS(=O)(=O)OC[C@H]1[C@H](F)[C@@H](Nc2ncncc2C(=O)c2cc3c(s2)CCC[C@]32OCCc3ccc(F)cc32)C[C@@H]1O] | 607 |
| 714 | 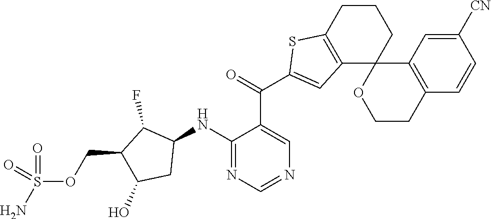 [SMILES: N#Cc1ccc2c(c1)C1(CCCc3sc(C(=O)c4cncnc4N[C@H]4C[C@H](O)[C@@H](COS(N)(=O)=O)[C@@H]4F)cc31)OCC2] | 614 |
| 715 | 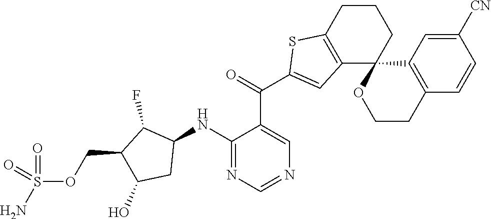 [SMILES: N#Cc1ccc2c(c1)[C@]1(CCCc3sc(C(=O)c4cncnc4N[C@H]4C[C@H](O)[C@@H](COS(N)(=O)=O)[C@@H]4F)cc31)OCC2] | 614 |
| 716 | 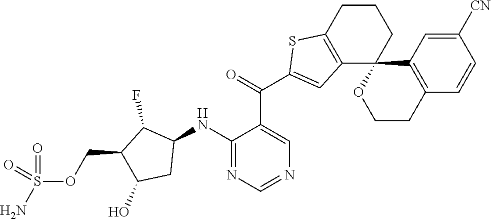 [SMILES: N#Cc1ccc2c(c1)[C@@]1(CCCc3sc(C(=O)c4cncnc4N[C@H]4C[C@H](O)[C@@H](COS(N)(=O)=O)[C@@H]4F)cc31)OCC2] | 614 |
| 717 | 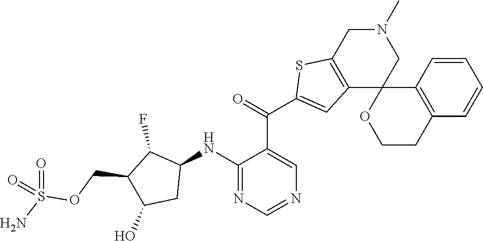 [SMILES: CN1Cc2sc(C(=O)c3cncnc3N[C@H]3C[C@H](O)[C@@H](COS(N)(=O)=O)[C@@H]3F)cc2C2(C1)OCCc1ccccc12] | 604 |
| 718 | 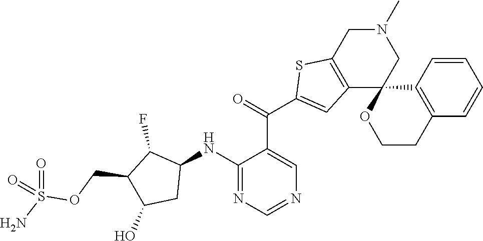 [SMILES: CN1Cc2sc(C(=O)c3cncnc3N[C@H]3C[C@H](O)[C@@H](COS(N)(=O)=O)[C@@H]3F)cc2[C@@]2(C1)OCCc1ccccc12] | 604 |
| 719 | 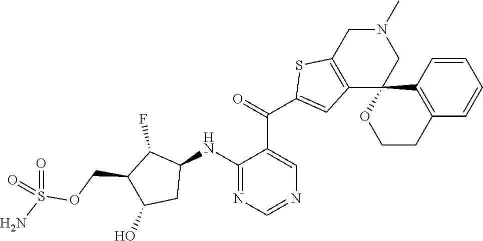 [SMILES: CN1Cc2sc(C(=O)c3cncnc3N[C@H]3C[C@H](O)[C@@H](COS(N)(=O)=O)[C@@H]3F)cc2[C@]2(C1)OCCc1ccccc12] | 604 |
| 720 | 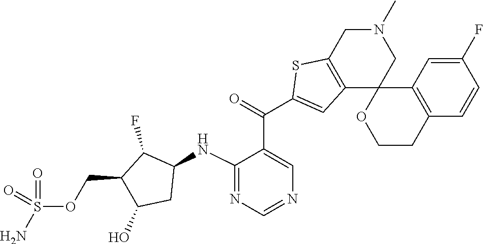 [SMILES: CN1Cc2sc(C(=O)c3cncnc3N[C@H]3C[C@H](O)[C@@H](COS(N)(=O)=O)[C@@H]3F)cc2C2(C1)OCCc1ccc(F)cc12] | 622 |
| 721 | 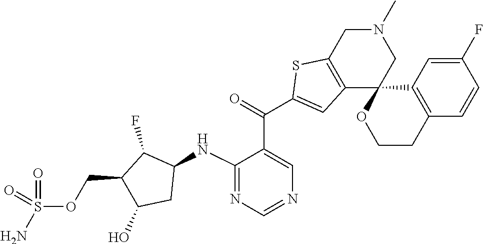 [SMILES: CN1Cc2sc(C(=O)c3cncnc3N[C@H]3C[C@H](O)[C@@H](COS(N)(=O)=O)[C@@H]3F)cc2[C@@]2(C1)OCCc1ccc(F)cc12] | 622 |
| 722 | 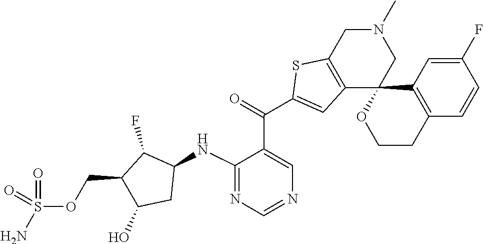 [SMILES: CN1Cc2sc(C(=O)c3cncnc3N[C@H]3C[C@H](O)[C@@H](COS(N)(=O)=O)[C@@H]3F)cc2[C@]2(C1)OCCc1ccc(F)cc12] | 622 |
| 723 | 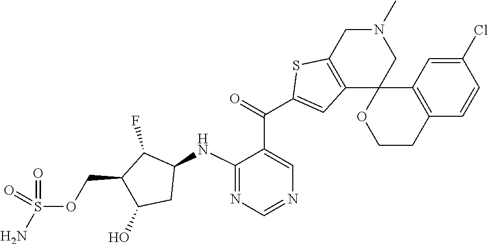 [SMILES: CN1Cc2sc(C(=O)c3cncnc3N[C@H]3C[C@H](O)[C@@H](COS(N)(=O)=O)[C@@H]3F)cc2C2(C1)OCCc1ccc(Cl)cc12] | 638 |
| 724 | 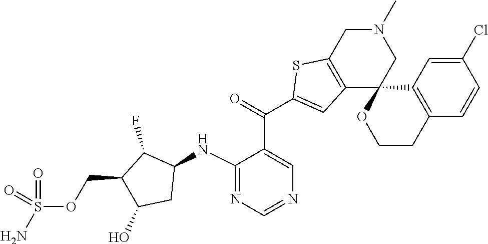 [SMILES: CN1Cc2sc(C(=O)c3cncnc3N[C@H]3C[C@H](O)[C@@H](COS(N)(=O)=O)[C@@H]3F)cc2[C@@]2(C1)OCCc1ccc(Cl)cc12] | 638 |
| 725 | 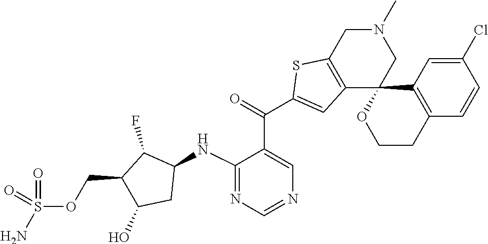 [SMILES: CN1Cc2sc(C(=O)c3cncnc3N[C@H]3C[C@H](O)[C@@H](COS(N)(=O)=O)[C@@H]3F)cc2[C@]2(C1)OCCc1ccc(Cl)cc12] | 638 |
| 726 | 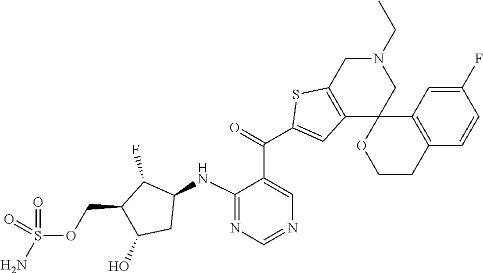 [SMILES: CCN1Cc2sc(C(=O)c3cncnc3N[C@H]3C[C@H](O)[C@@H](COS(N)(=O)=O)[C@@H]3F)cc2C2(C1)OCCc1ccc(F)cc12] | 636 |
| 727 | 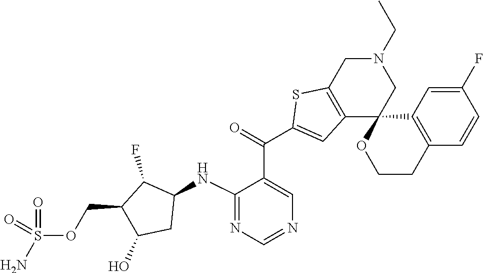 [SMILES: CCN1Cc2sc(C(=O)c3cncnc3N[C@H]3C[C@H](O)[C@@H](COS(N)(=O)=O)[C@@H]3F)cc2[C@@]2(C1)OCCc1ccc(F)cc12] | 636 |
| 728 | 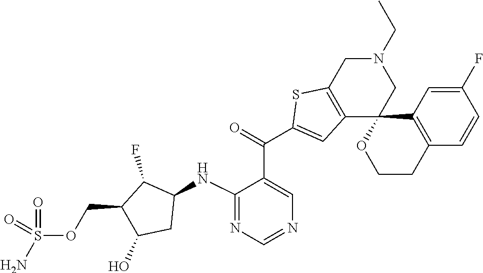 [SMILES: CCN1Cc2sc(C(=O)c3cncnc3N[C@H]3C[C@H](O)[C@@H](COS(N)(=O)=O)[C@@H]3F)cc2[C@]2(C1)OCCc1ccc(F)cc12] | 636 |
| 729 | 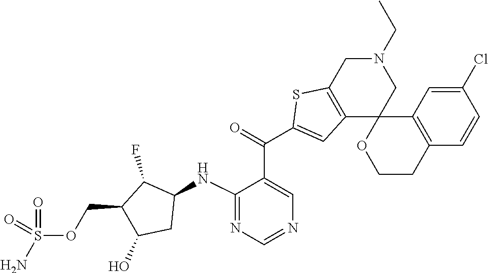 [SMILES: CCN1Cc2sc(C(=O)c3cncnc3N[C@H]3C[C@H](O)[C@@H](COS(N)(=O)=O)[C@@H]3F)cc2C2(C1)OCCc1ccc(Cl)cc12] | 652 |
| 730 | 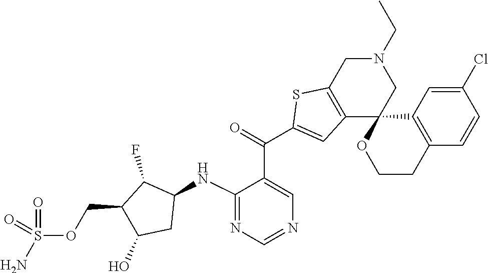 [SMILES: CCN1Cc2sc(C(=O)c3cncnc3N[C@H]3C[C@H](O)[C@@H](COS(N)(=O)=O)[C@@H]3F)cc2[C@@]2(C1)OCCc1ccc(Cl)cc12] | 652 |
| 731 | 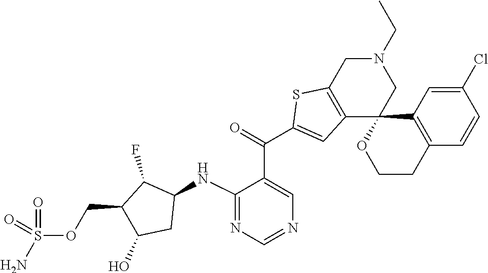 [SMILES: CCN1Cc2sc(C(=O)c3cncnc3N[C@H]3C[C@H](O)[C@@H](COS(N)(=O)=O)[C@@H]3F)cc2[C@]2(C1)OCCc1ccc(Cl)cc12] | 652 |
| 732 | 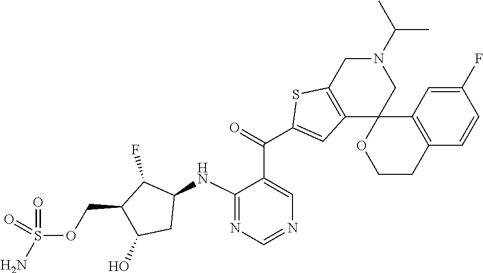 [SMILES: CC(C)N1Cc2sc(C(=O)c3cncnc3N[C@H]3C[C@H](O)[C@@H](COS(N)(=O)=O)[C@@H]3F)cc2C2(C1)OCCc1ccc(F)cc12] | 650 |
| 733 | 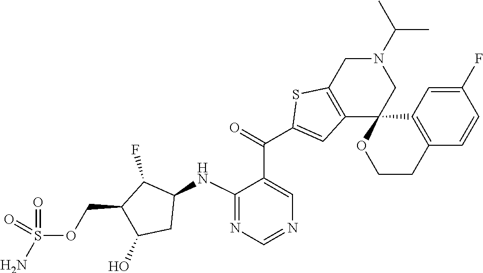 [SMILES: CC(C)N1Cc2sc(C(=O)c3cncnc3N[C@H]3C[C@H](O)[C@@H](COS(N)(=O)=O)[C@@H]3F)cc2[C@@]2(C1)OCCc1ccc(F)cc12] | 650 |
| 734 | 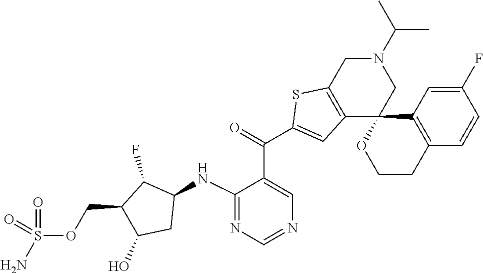 [SMILES: CC(C)N1Cc2sc(C(=O)c3cncnc3N[C@H]3C[C@H](O)[C@@H](COS(N)(=O)=O)[C@@H]3F)cc2[C@]2(C1)OCCc1ccc(F)cc12] | 650 |
| 735 | 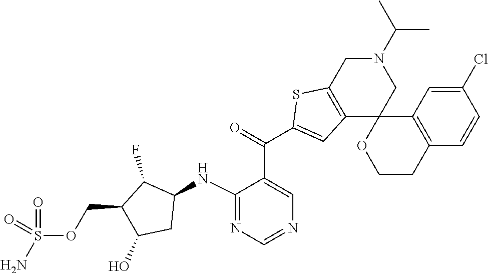 [SMILES: CC(C)N1Cc2sc(C(=O)c3cncnc3N[C@H]3C[C@H](O)[C@@H](COS(N)(=O)=O)[C@@H]3F)cc2C2(C1)OCCc1ccc(Cl)cc12] | 666 |
| 736 | 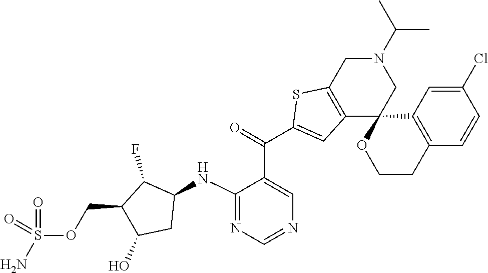 [SMILES: CC(C)N1Cc2sc(C(=O)c3cncnc3N[C@H]3C[C@H](O)[C@@H](COS(N)(=O)=O)[C@@H]3F)cc2[C@@]2(C1)OCCc1ccc(Cl)cc12] | 666 |
| 737 | 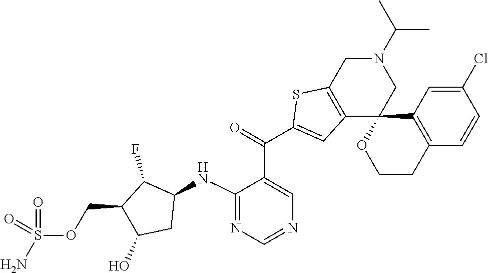 [SMILES: CC(C)N1Cc2sc(C(=O)c3cncnc3N[C@H]3C[C@H](O)[C@@H](COS(N)(=O)=O)[C@@H]3F)cc2[C@]2(C1)OCCc1ccc(Cl)cc12] | 666 |
| 738 | 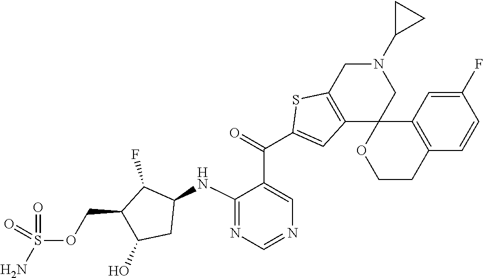 [SMILES: NS(=O)(=O)OC[C@H]1[C@H](F)[C@@H](Nc2ncncc2C(=O)c2cc3c(s2)CN(C2CC2)CC32OCCc3ccc(F)cc32)C[C@@H]1O] | 648 |
| 739 | 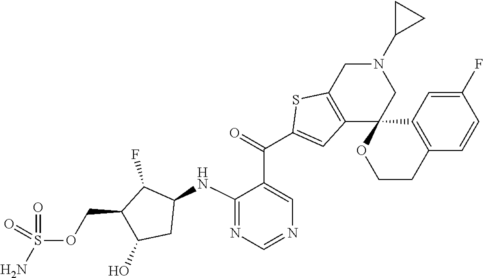 [SMILES: NS(=O)(=O)OC[C@H]1[C@H](F)[C@@H](Nc2ncncc2C(=O)c2cc3c(s2)CN(C2CC2)C[C@@]32OCCc3ccc(F)cc32)C[C@@H]1O] | 648 |
| 740 | 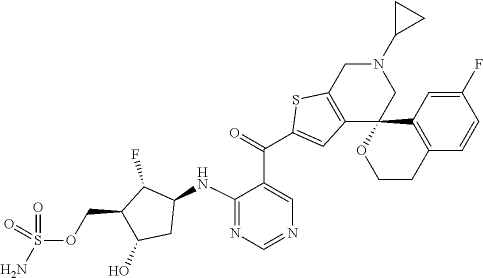 [SMILES: NS(=O)(=O)OC[C@H]1[C@H](F)[C@@H](Nc2ncncc2C(=O)c2cc3c(s2)CN(C2CC2)C[C@]32OCCc3ccc(F)cc32)C[C@@H]1O] | 648 |
| 741 | 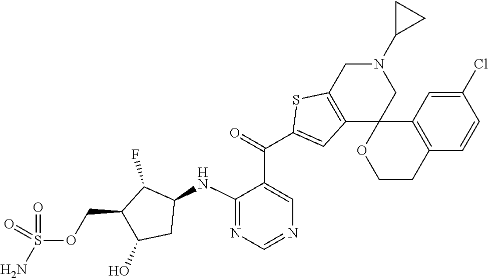 [SMILES: NS(=O)(=O)OC[C@H]1[C@H](F)[C@@H](Nc2ncncc2C(=O)c2cc3c(s2)CN(C2CC2)CC32OCCc3ccc(Cl)cc32)C[C@@H]1O] | 664 |
| 742 | 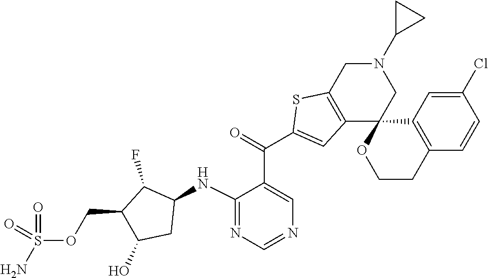 [SMILES: NS(=O)(=O)OC[C@H]1[C@H](F)[C@@H](Nc2ncncc2C(=O)c2cc3c(s2)CN(C2CC2)C[C@@]32OCCc3ccc(Cl)cc32)C[C@@H]1O] | 664 |
| 743 | 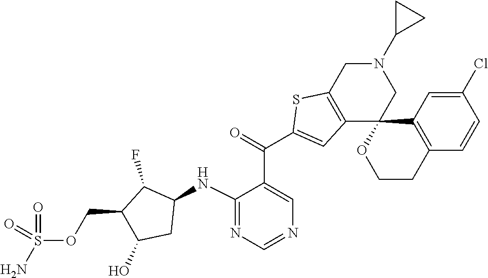 [SMILES: NS(=O)(=O)OC[C@H]1[C@H](F)[C@@H](Nc2ncncc2C(=O)c2cc3c(s2)CN(C2CC2)C[C@]32OCCc3ccc(Cl)cc32)C[C@@H]1O] | 664 |
| 744 | 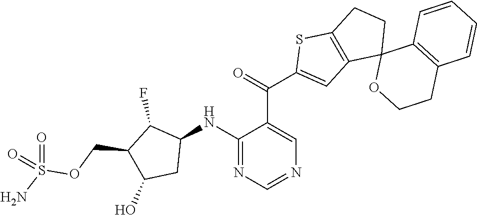 [SMILES: NS(=O)(=O)OC[C@H]1[C@H](F)[C@@H](Nc2ncncc2C(=O)c2cc3c(s2)CCC32OCCc3ccccc32)C[C@@H]1O] | 575 |
| 745 | 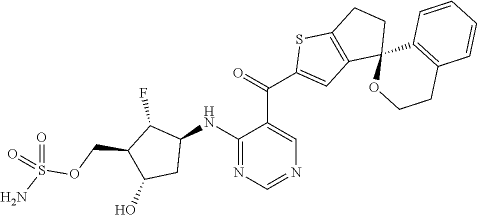 [SMILES: NS(=O)(=O)OC[C@H]1[C@H](F)[C@@H](Nc2ncncc2C(=O)c2cc3c(s2)CC[C@@]32OCCc3ccccc32)C[C@@H]1O] | 575 |
| 746 | 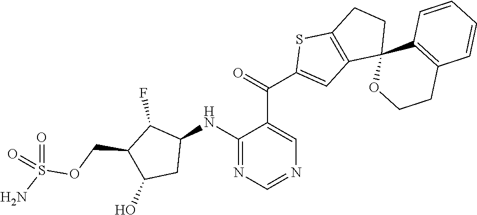 [SMILES: NS(=O)(=O)OC[C@H]1[C@H](F)[C@@H](Nc2ncncc2C(=O)c2cc3c(s2)CC[C@]32OCCc3ccccc32)C[C@@H]1O] | 575 |
| 747 | 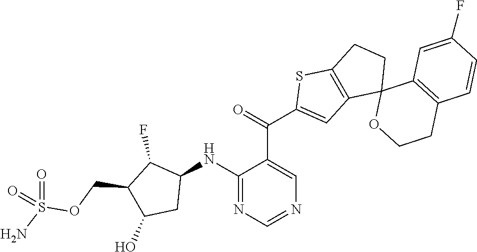 [SMILES: NS(=O)(=O)OC[C@H]1[C@H](F)[C@@H](Nc2ncncc2C(=O)c2cc3c(s2)CCC32OCCc3ccc(F)cc32)C[C@@H]1O] | 593 |
| 748 | 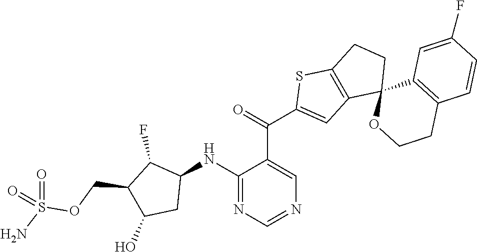 [SMILES: NS(=O)(=O)OC[C@H]1[C@H](F)[C@@H](Nc2ncncc2C(=O)c2cc3c(s2)CC[C@@]32OCCc3ccc(F)cc32)C[C@@H]1O] | 593 |
| 749 | 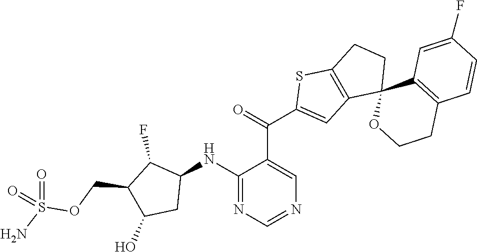 [SMILES: NS(=O)(=O)OC[C@H]1[C@H](F)[C@@H](Nc2ncncc2C(=O)c2cc3c(s2)CC[C@]32OCCc3ccc(F)cc32)C[C@@H]1O] | 593 |
| 750 | 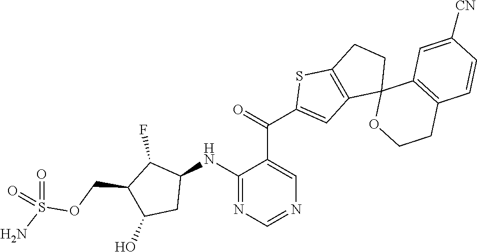 [SMILES: N#Cc1ccc2c(c1)C1(CCc3sc(C(=O)c4cncnc4N[C@H]4C[C@H](O)[C@@H](COS(N)(=O)=O)[C@@H]4F)cc31)OCC2] | 600 |
| 751 | 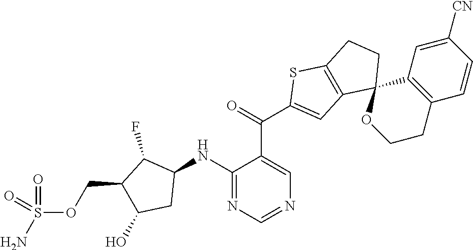 [SMILES: N#Cc1ccc2c(c1)[C@]1(CCc3sc(C(=O)c4cncnc4N[C@H]4C[C@H](O)[C@@H](COS(N)(=O)=O)[C@@H]4F)cc31)OCC2] | 600 |
| 752 | 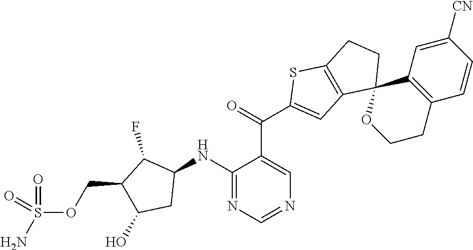 [SMILES: N#Cc1ccc2c(c1)[C@@]1(CCc3sc(C(=O)c4cncnc4N[C@H]4C[C@H](O)[C@@H](COS(N)(=O)=O)[C@@H]4F)cc31)OCC2] | 600 |
| 753 | 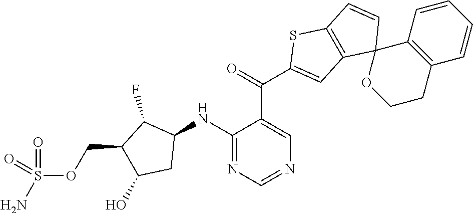 [SMILES: NS(=O)(=O)OC[C@H]1[C@H](F)[C@@H](Nc2ncncc2C(=O)c2cc3c(s2)C=CC32OCCc3ccccc32)C[C@@H]1O] | 573 |
| 754 | 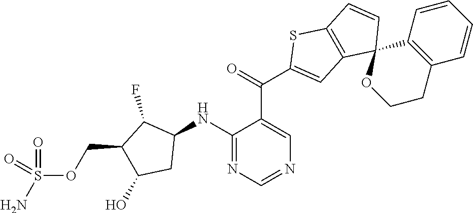 [SMILES: NS(=O)(=O)OC[C@H]1[C@H](F)[C@@H](Nc2ncncc2C(=O)c2cc3c(s2)C=C[C@@]32OCCc3ccccc32)C[C@@H]1O] | 573 |
| 755 | 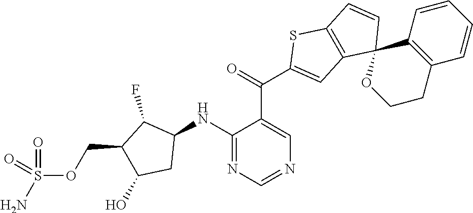 [SMILES: NS(=O)(=O)OC[C@H]1[C@H](F)[C@@H](Nc2ncncc2C(=O)c2cc3c(s2)C=C[C@]32OCCc3ccccc32)C[C@@H]1O] | 573 |
| 756 | 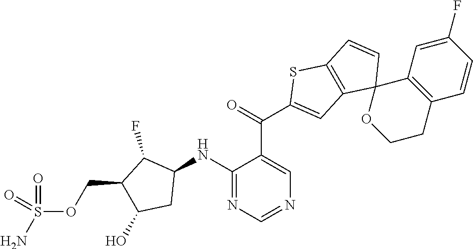 [SMILES: NS(=O)(=O)OC[C@H]1[C@H](F)[C@@H](Nc2ncncc2C(=O)c2cc3c(s2)C=CC32OCCc3ccc(F)cc32)C[C@@H]1O] | 591 |
| 757 | 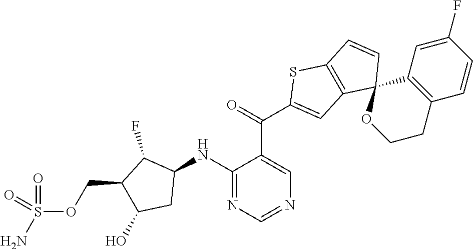 [SMILES: NS(=O)(=O)OC[C@H]1[C@H](F)[C@@H](Nc2ncncc2C(=O)c2cc3c(s2)C=C[C@@]32OCCc3ccc(F)cc32)C[C@@H]1O] | 591 |
| 758 | 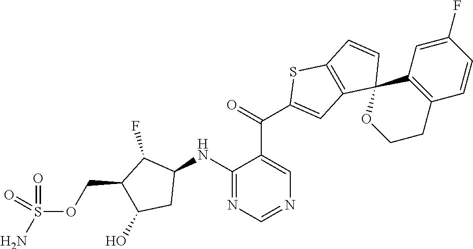 [SMILES: NS(=O)(=O)OC[C@H]1[C@H](F)[C@@H](Nc2ncncc2C(=O)c2cc3c(s2)C=C[C@]32OCCc3ccc(F)cc32)C[C@@H]1O] | 591 |
| 759 | 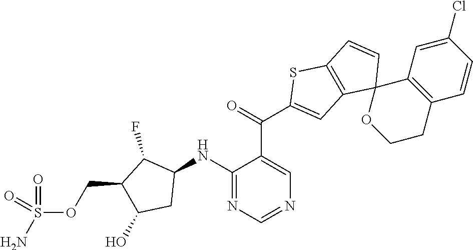 [SMILES: NS(=O)(=O)OC[C@H]1[C@H](F)[C@@H](Nc2ncncc2C(=O)c2cc3c(s2)C=CC32OCCc3ccc(Cl)cc32)C[C@@H]1O] | 607 |
| 760 | 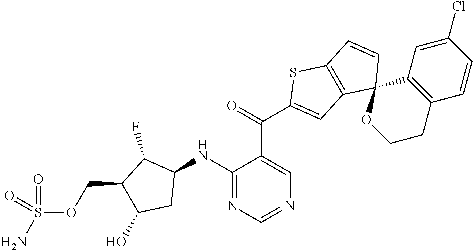 [SMILES: NS(=O)(=O)OC[C@H]1[C@H](F)[C@@H](Nc2ncncc2C(=O)c2cc3c(s2)C=C[C@@]32OCCc3ccc(Cl)cc32)C[C@@H]1O] | 607 |
| 761 | 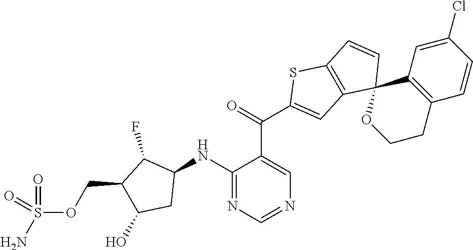 [SMILES: NS(=O)(=O)OC[C@H]1[C@H](F)[C@@H](Nc2ncncc2C(=O)c2cc3c(s2)C=C[C@]32OCCc3ccc(Cl)cc32)C[C@@H]1O] | 607 |
| 762 | 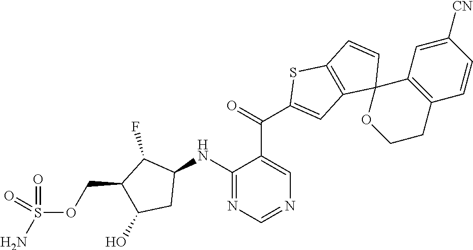 [SMILES: N#Cc1ccc2c(c1)C1(C=Cc3sc(C(=O)c4cncnc4N[C@H]4C[C@H](O)[C@@H](COS(N)(=O)=O)[C@@H]4F)cc31)OCC2] | 598 |
| 763 | 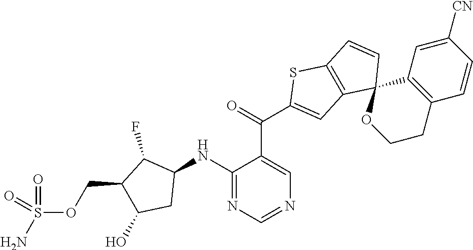 [SMILES: N#Cc1ccc2c(c1)[C@]1(C=Cc3sc(C(=O)c4cncnc4N[C@H]4C[C@H](O)[C@@H](COS(N)(=O)=O)[C@@H]4F)cc31)OCC2] | 598 |
| 764 | 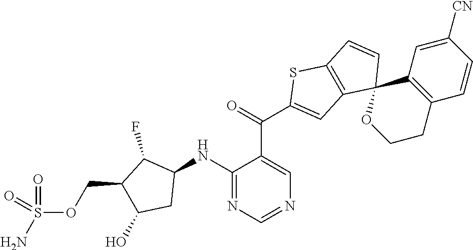 [SMILES: N#Cc1ccc2c(c1)[C@@]1(C=Cc3sc(C(=O)c4cncnc4N[C@H]4C[C@H](O)[C@@H](COS(N)(=O)=O)[C@@H]4F)cc31)OCC2] | 598 |
| 765 | 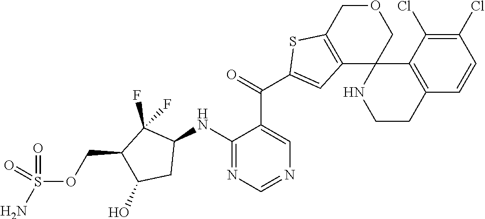 | 676 |
| 766 | 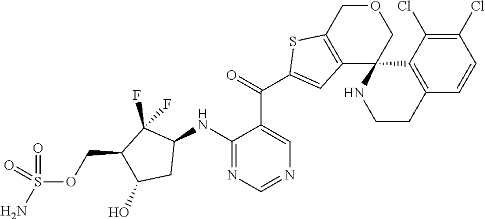 | 676 |
| 767 | 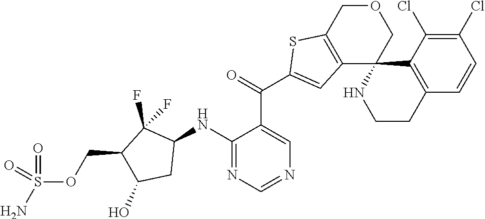 | 676 |
| 768 | 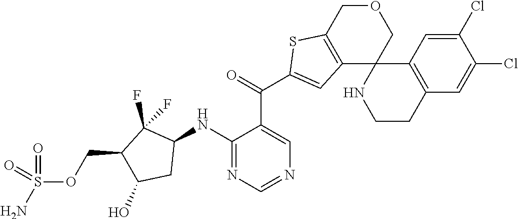 | 676 |
| 769 | 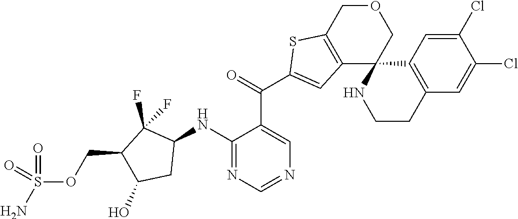 | 676 |
| 770 | 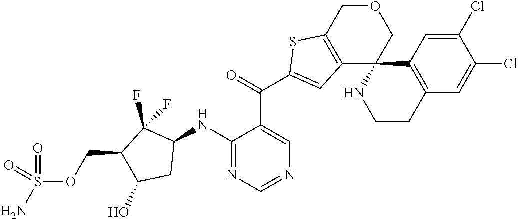 | 676 |
| 771 | 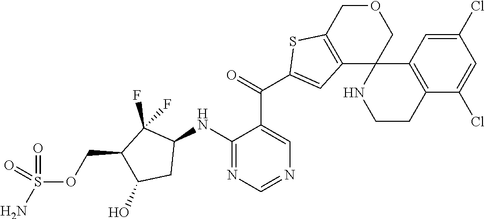 | 676 |
| 772 | 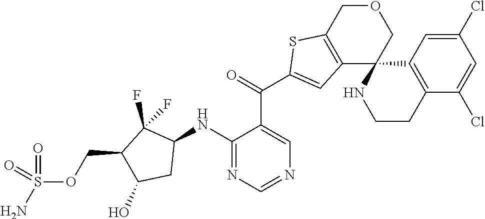 | 676 |
| 773 | 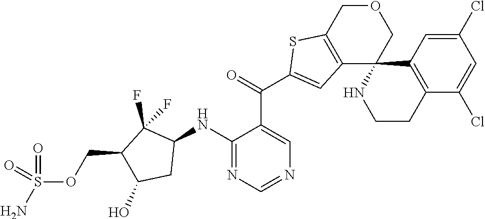 | 676 |
| 774 | 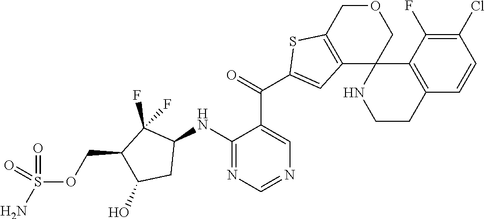 | 660 |
| 775 | 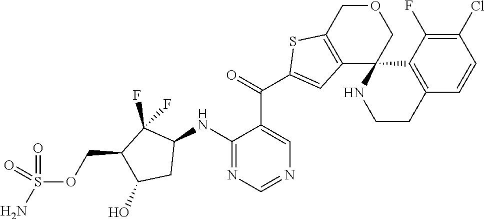 | 660 |
| 776 | 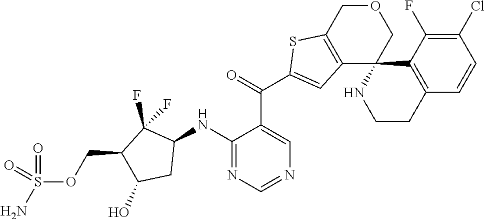 | 660 |
| 777 | 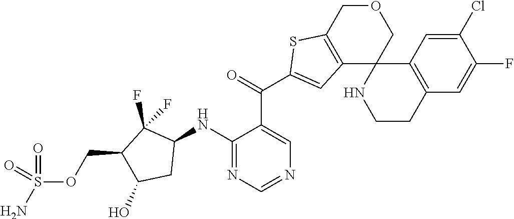 | 660 |
| 778 | 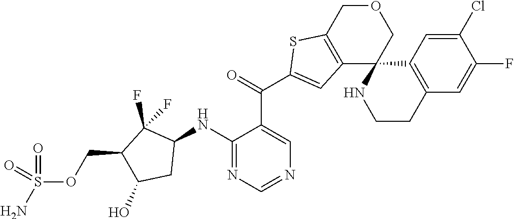 | 660 |
| 779 | 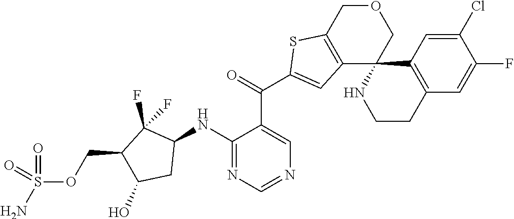 | 660 |
| 780 | 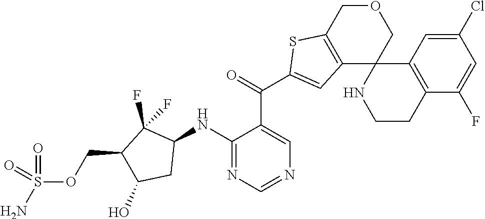 | 660 |
| 781 | 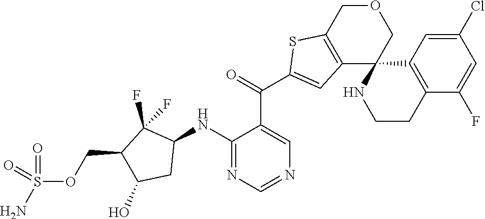 | 660 |
| 782 | 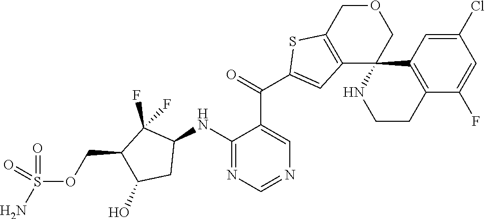 | 660 |
| 783 | 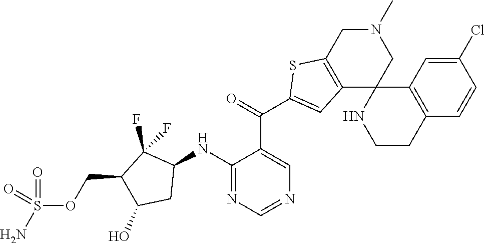 | 655 |
| 784 | 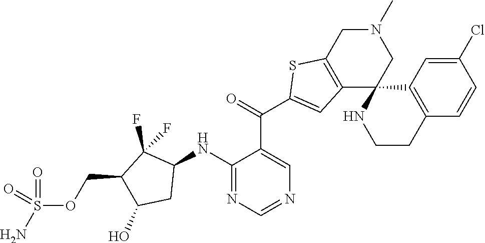 | 655 |
| 785 | 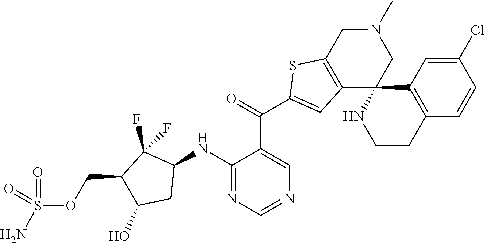 | 655 |
| 786 | 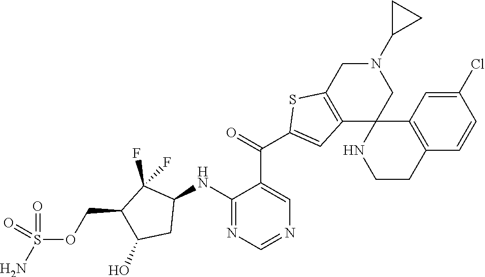 | 681 |
| 787 | 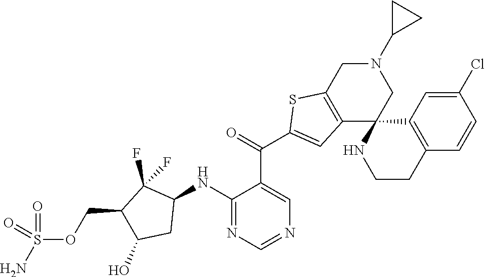 | 681 |
| 788 | 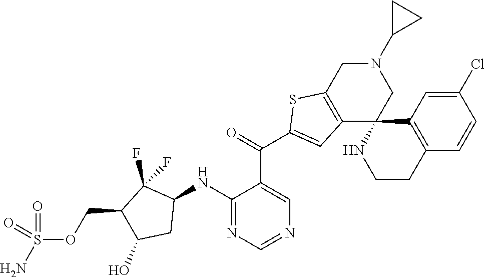 | 681 |
| 789 | 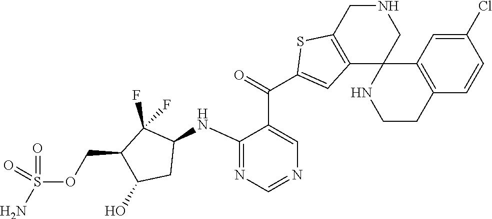 | 641 |
| 790 | 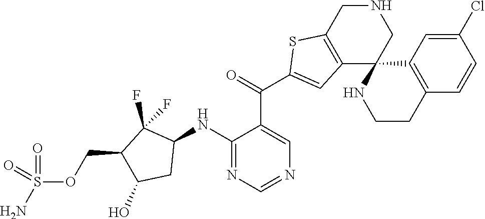 | 641 |
| 791 | 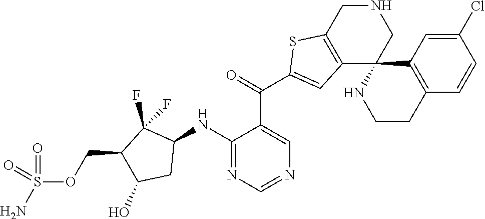 | 64 |
| 792 | 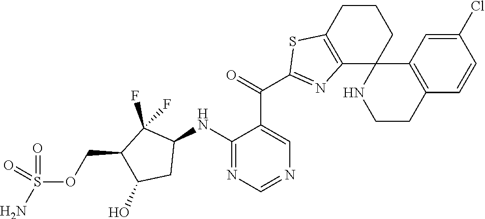 | 641 |
| 793 | 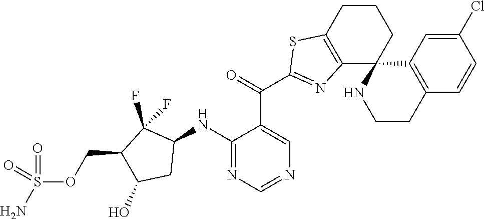 | 641 |
| 794 | 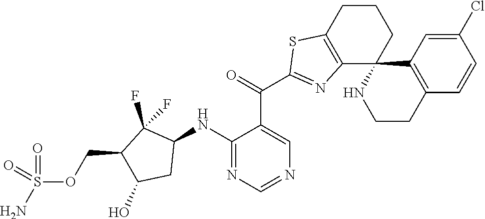 | 641 |
| 795 | 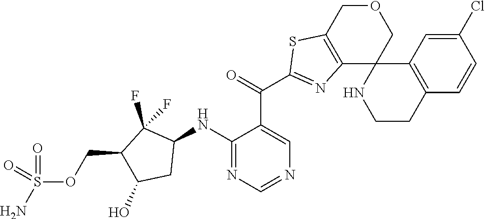 | 643 |
| 796 | 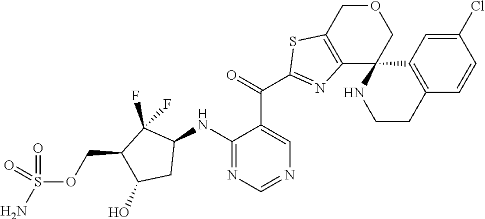 | 643 |
| 797 | 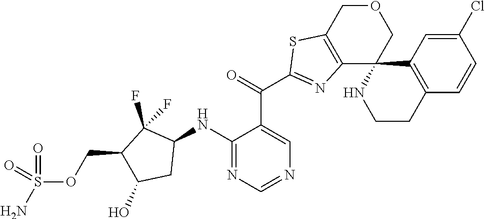 | 643 |
| 798 | 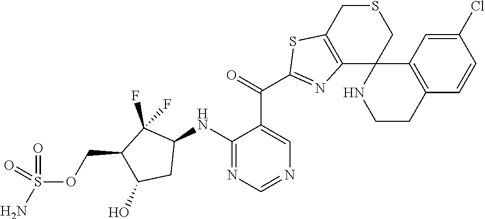 | 659 |
| 799 | 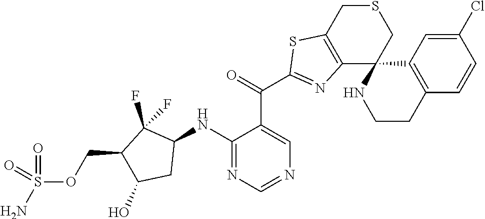 | 659 |
| 800 | 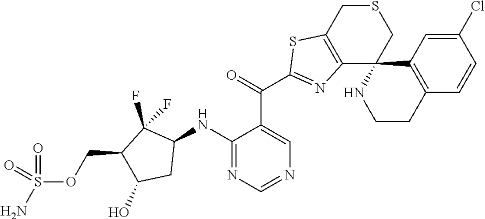 | 659 |
| 801 | 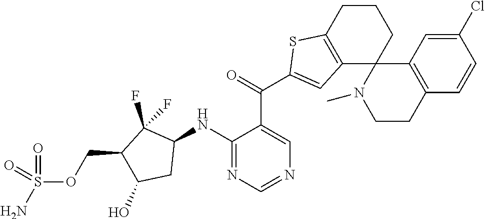 | 654 |
| 802 | 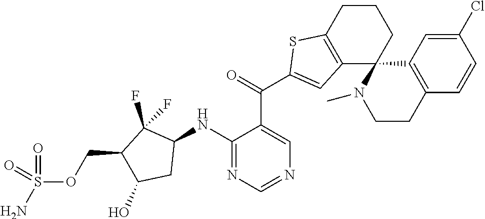 | 654 |
| 803 | 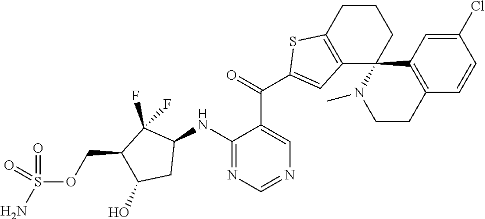 | 654 |
| 804 | 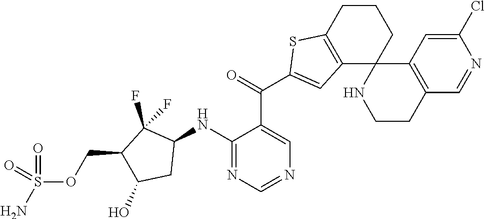 | 641 |
| 805 | 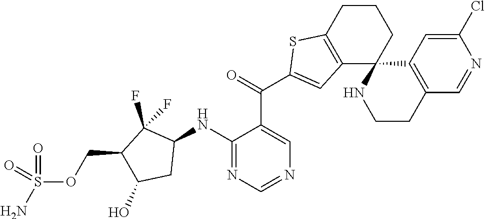 | 641 |
| 806 | 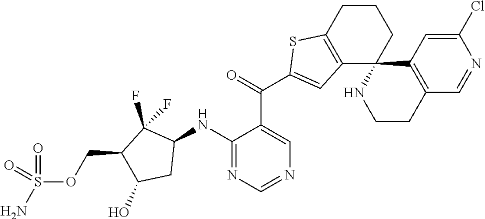 | 641 |
| 807 | 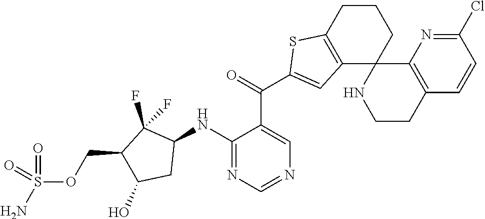 | 641 |
| 808 | 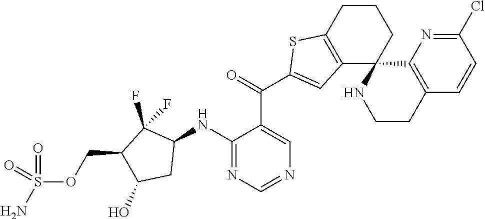 | 641 |
| 809 | 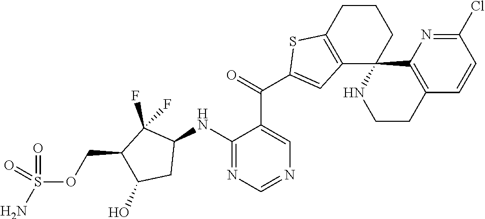 | 641 |
| 810 | 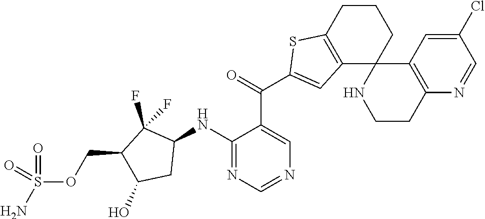 | 641 |
| 811 | 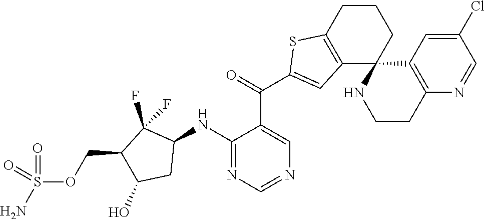 | 641 |
| 812 | 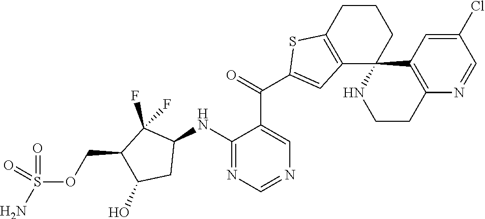 | 641 |
| 813 | 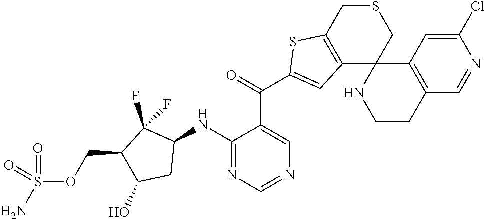 | 659 |
| 814 | 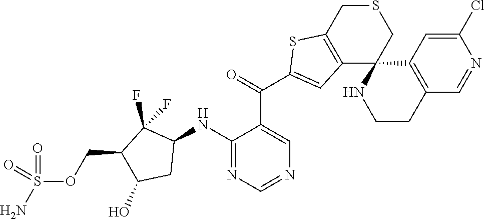 | 659 |
| 815 | 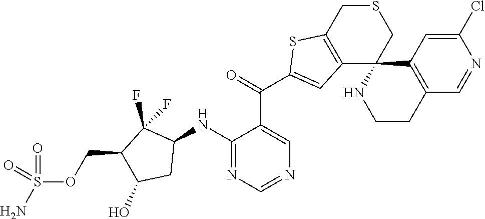 | 659 |
| 816 | 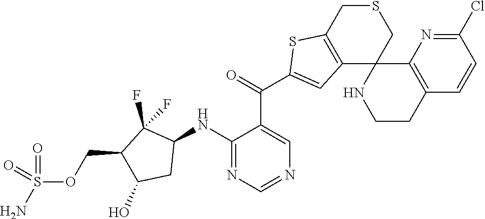 | 659 |
| 817 | 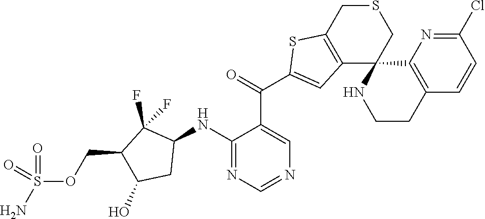 | 659 |
| 818 | 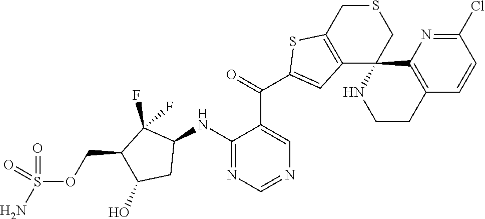 | 659 |
| 819 | 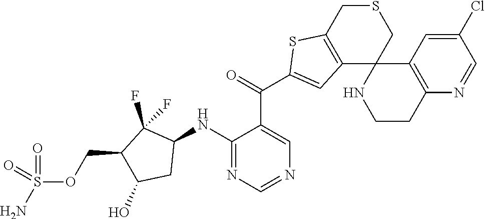 | 659 |
| 820 | 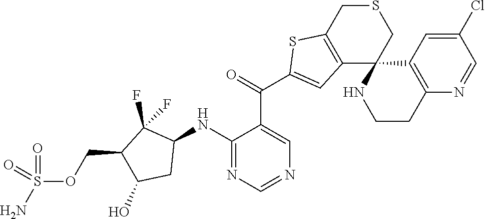 | 659 |
| 821 | 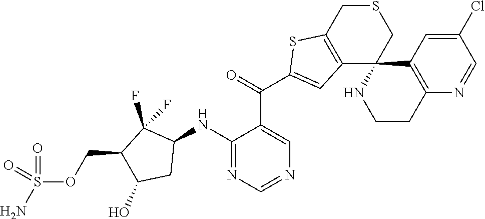 | 659 |
| 822 | 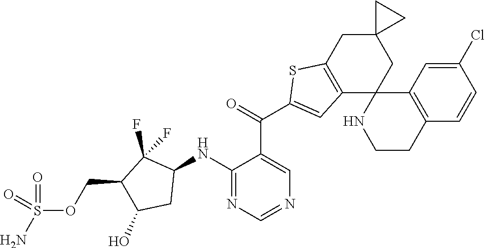 | 666 |
| 823 | 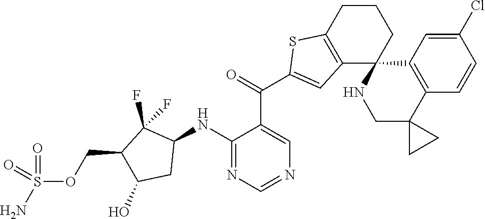 | 666 |
| 824 | 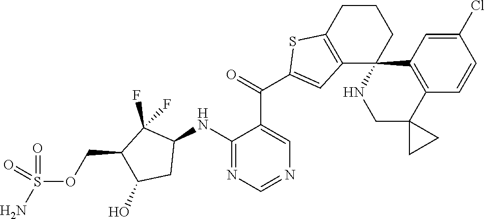 | 666 |
| 825 | 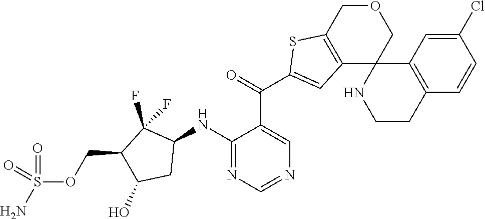 | 642 |
| 826 | 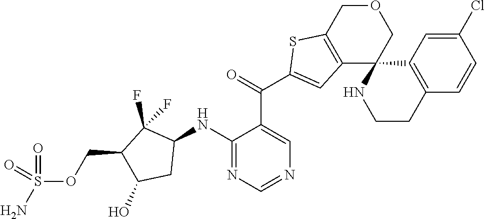 | 642 |
| 827 | 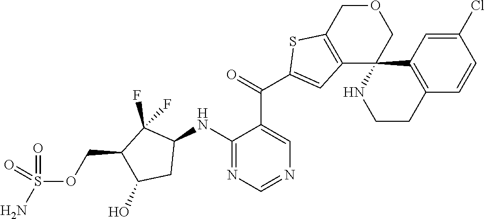 | 642 |
| 828 | 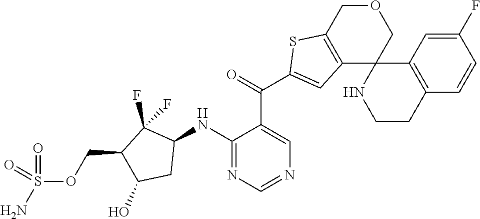 | 626 |
| 829 | 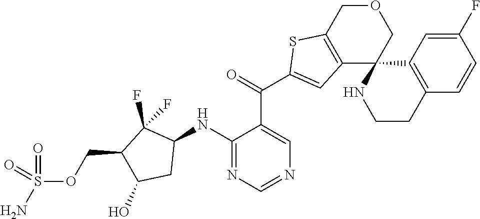 | 626 |
| 830 | 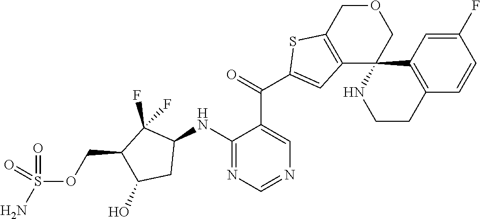 | 626 |
| 831 | 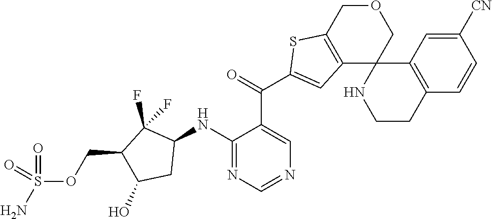 | 633 |
| 832 | 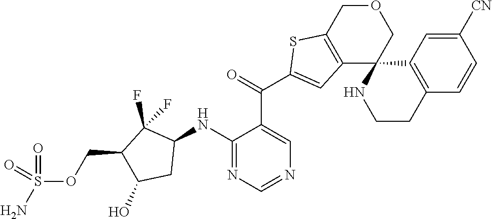 | 633 |
| 833 | 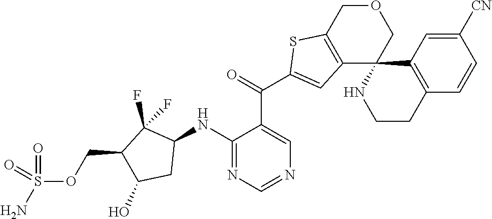 | 633 |
| 834 | 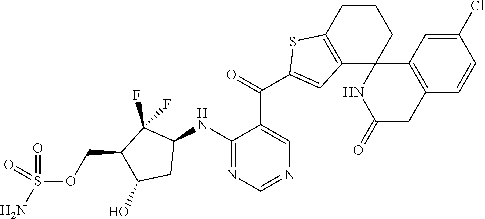 | 654 |
| 835 | 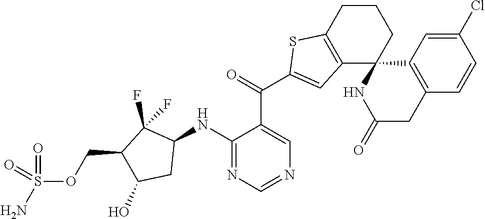 | 654 |
| 836 | 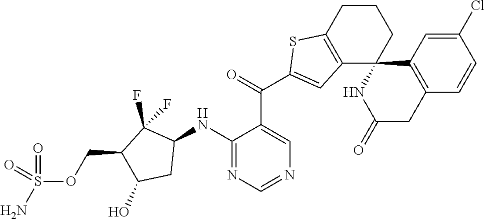 | 654 |
| 837 | 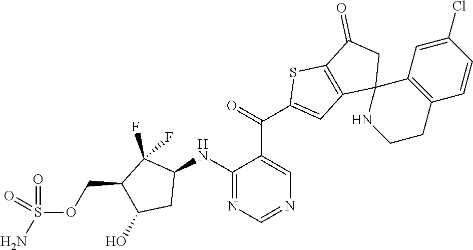 | 640 |
| 838 | 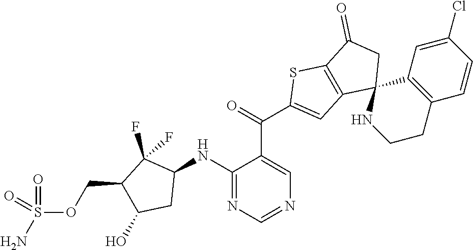 | 640 |
| 839 | 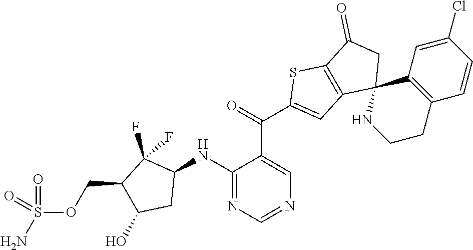 | 640 |
| 840 | 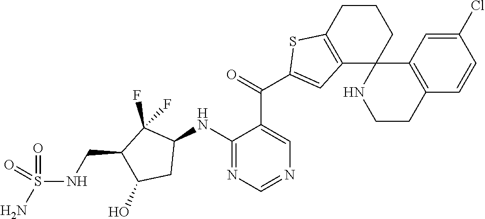 | 639 |
| 841 | 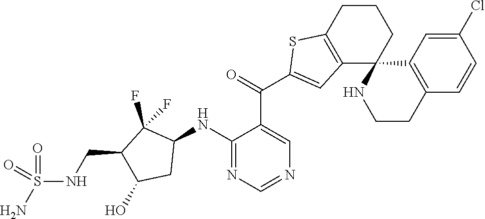 | 639 |
| 842 | 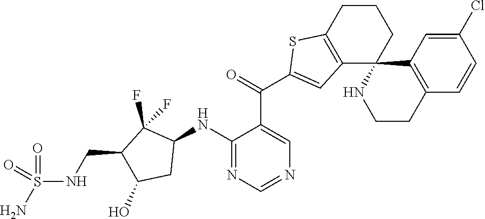 | 639 |
| 843 | 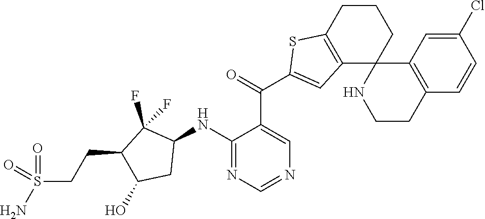 | 638 |
| 844 | 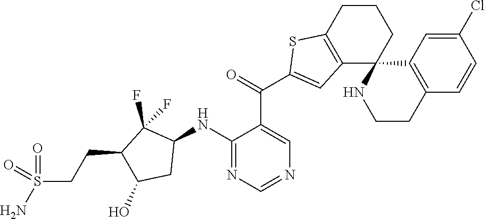 | 638 |
| 845 | 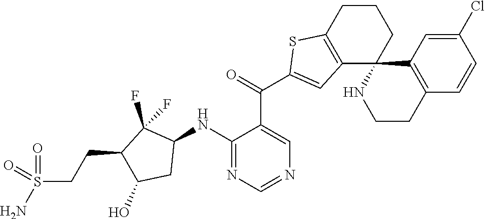 | 638 |
| 846 | 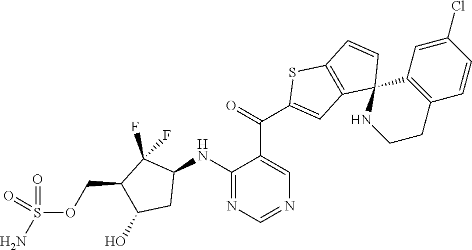 | 622 |
| 847 | 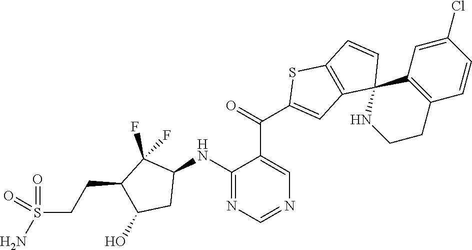 | 622 |
| 848 | 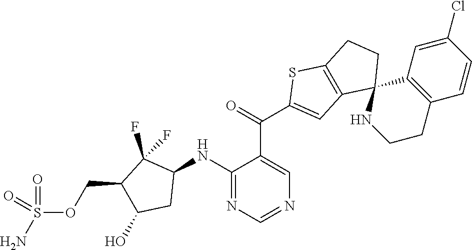 | 624 |
| 849 | 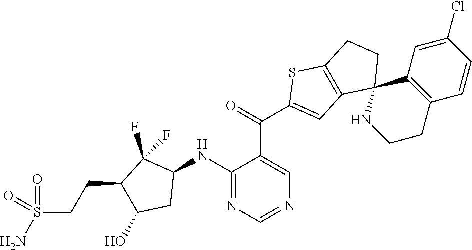 | 624 |
| 850 | 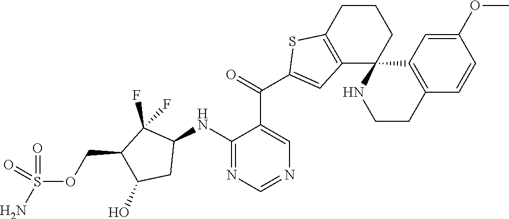 | 636 |
| 851 | 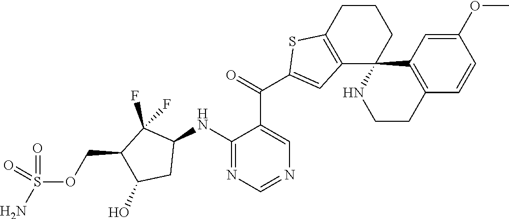 | 636 |
| 852 | 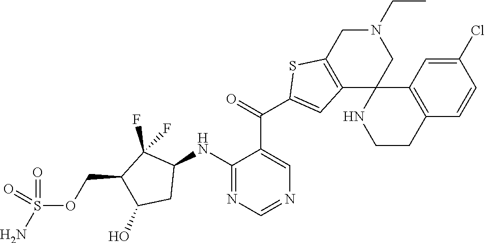 | 669 |
| 853 | 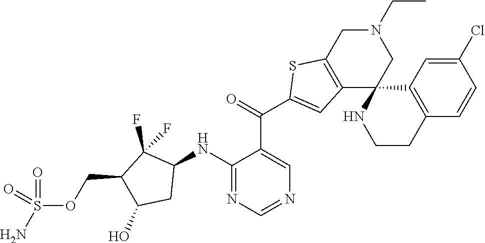 | 669 |
| 854 | 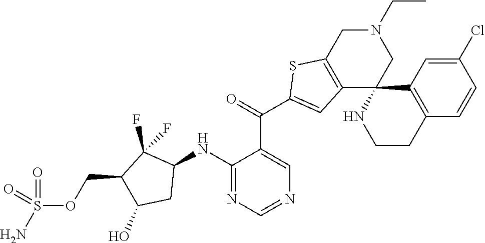 | 669 |
| 855 | 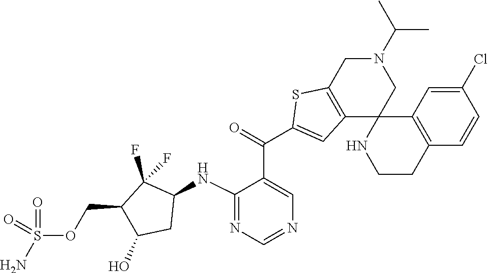 | 683 |
| 856 | 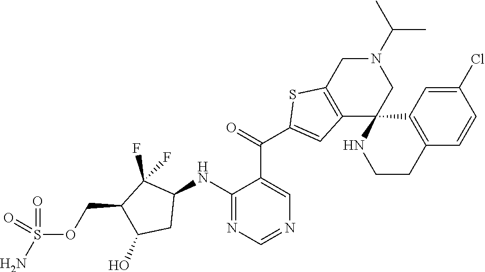 | 683 |
| 857 | 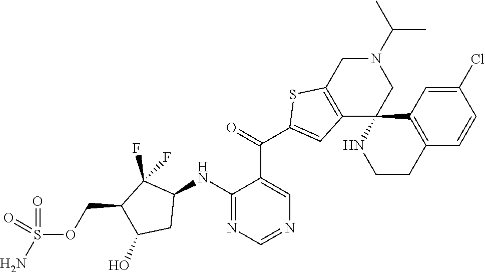 | 683 |
| 858 | 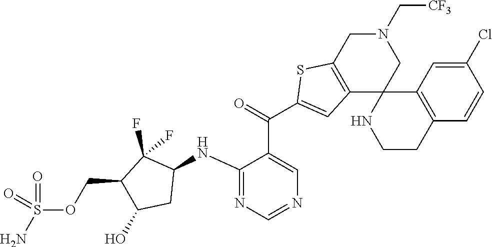 | 723 |
| 859 | 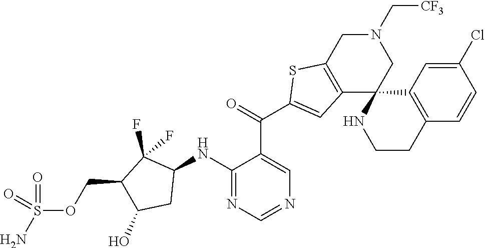 | 723 |
| 860 | 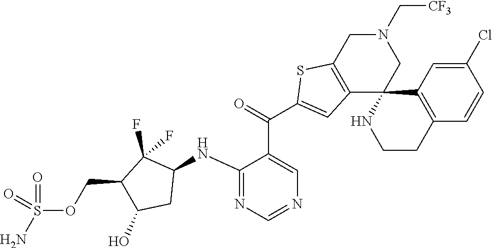 | 723 |
| 861 | 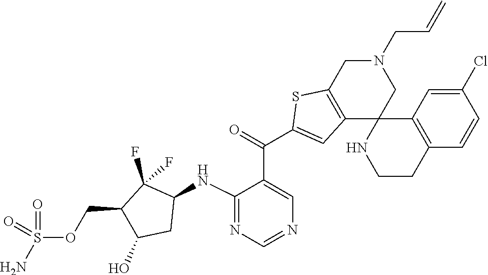 | 681 |
| 862 | 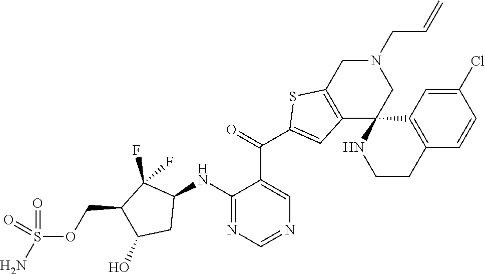 | 681 |
| 863 | 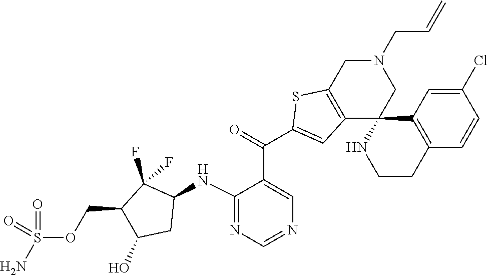 | 681 |
| 864 | 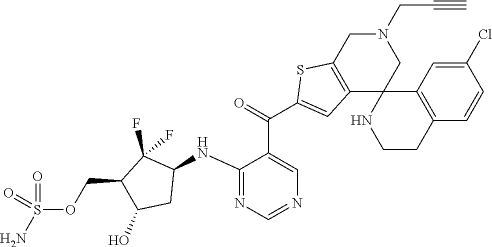 | 679 |
| 865 | 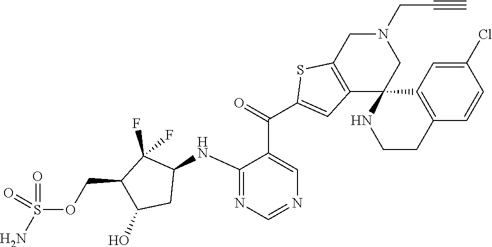 | 679 |
| 866 | 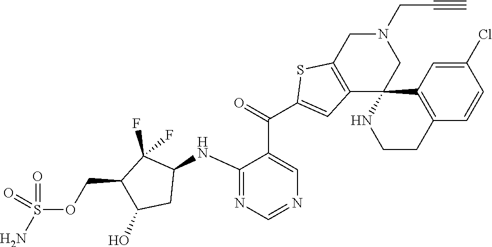 | 679 |
| 867 | 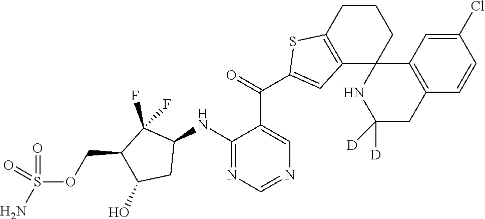 | 642 |
| 868 | 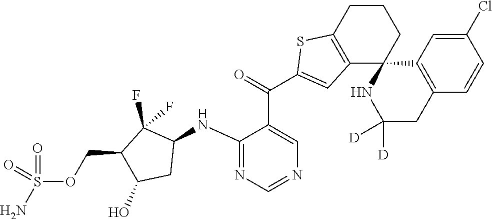 | 642 |
| 869 | 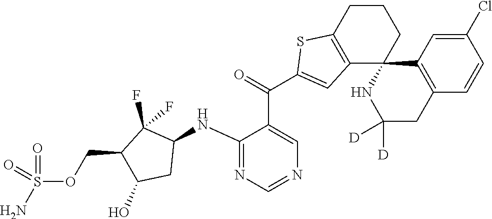 | 642 |
| 870 | 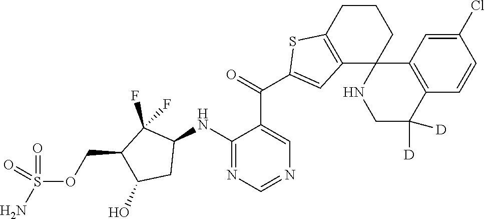 | 642 |
| 871 | 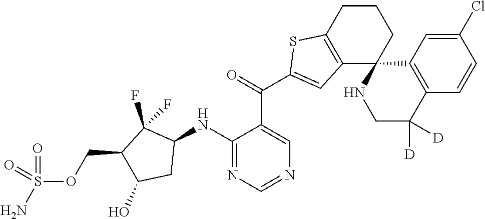 | 642 |
| 872 | 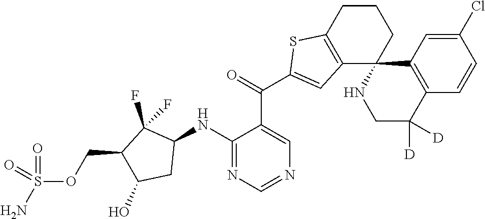 | 642 |
| 873 | 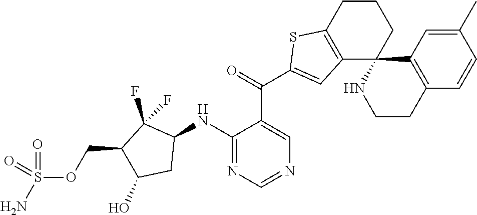 | 620 |
| 874 | 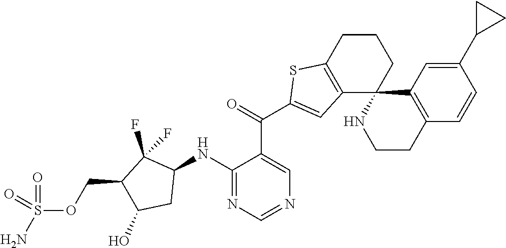 | 646 |
| 875 | 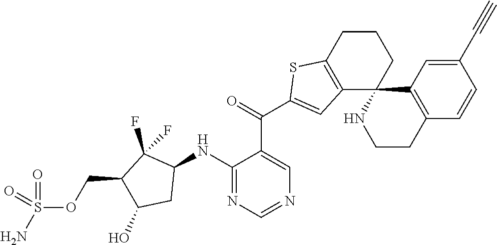 | 630 |
| 876 | 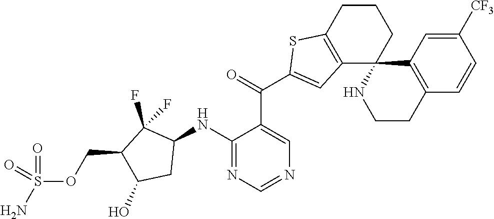 | 674 |
| 877 | 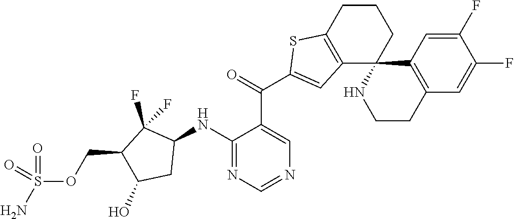 | 642 |
| 878 | 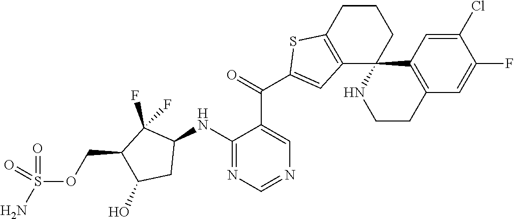 | 658 |
| 879 | 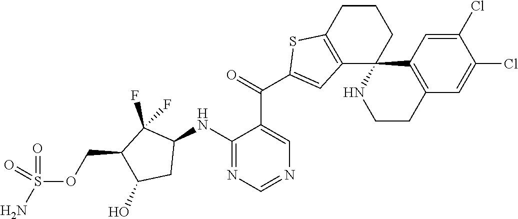 | 674 |
| 880 | 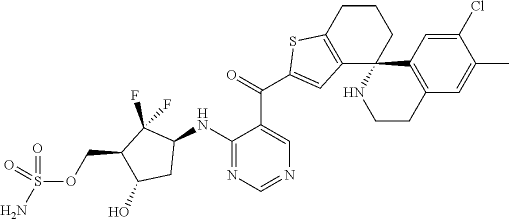 | 654 |
| 881 | 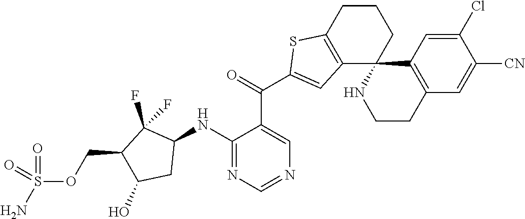 | 665 |
| 882 | 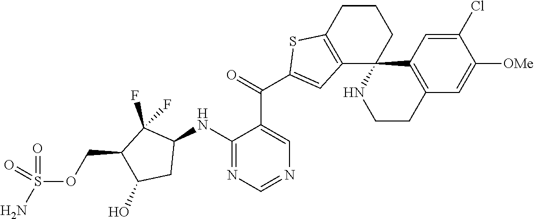 | 670 |
| 883 | 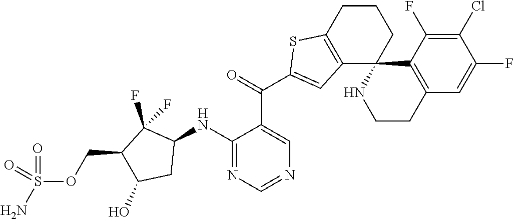 | 676 |
| 884 | 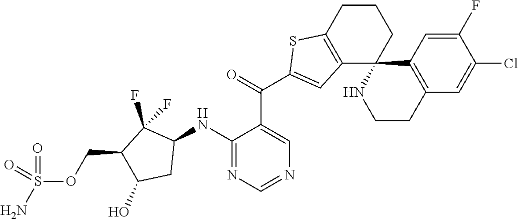 | 658 |
| 885 | 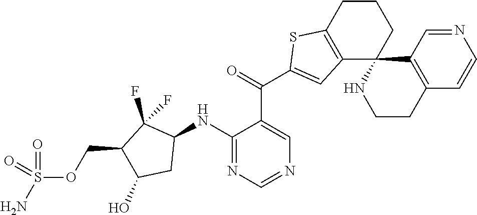 | 607 |
| 886 | 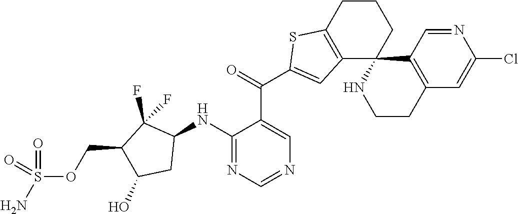 | 641 |
| 887 | 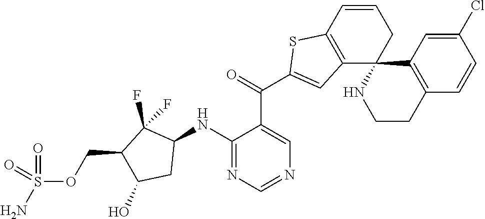 | 638 |
| 888 | 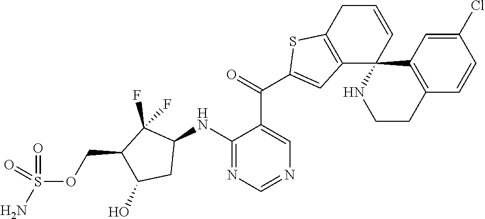 | 638 |
| 889 | 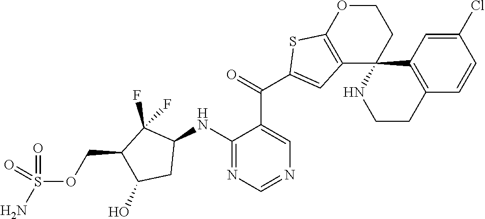 | 642 |
| 890 | 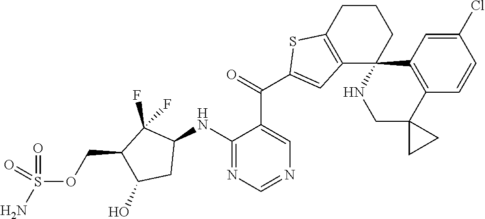 | 666 |
| 891 | 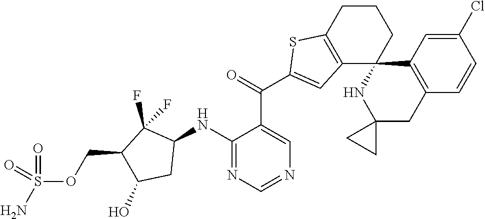 | 666 |
| 892 | 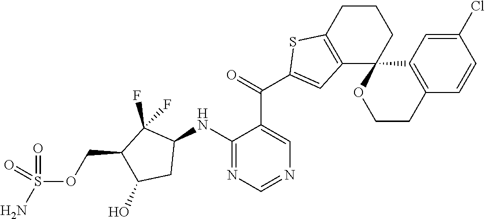 | 641 |
| 893 | 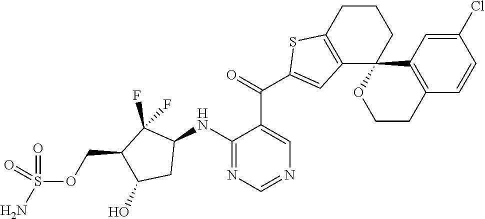 | 641 |
| 894 | 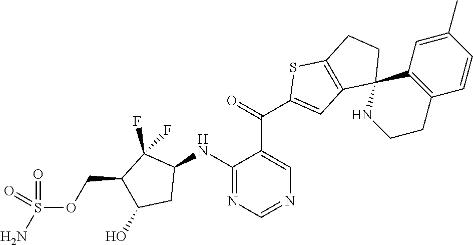 | 606 |
| 895 | 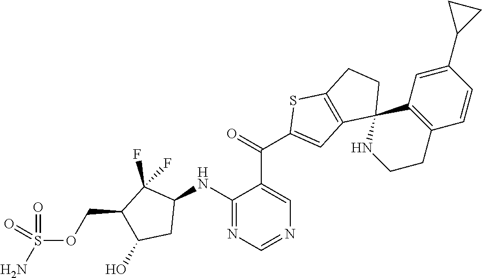 | 632 |
| 896 | 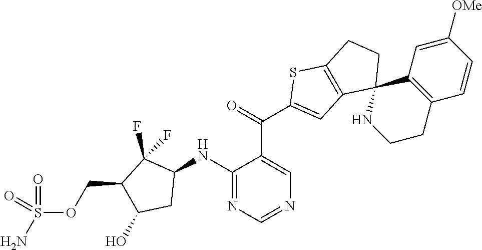 | 622 |
| 897 | 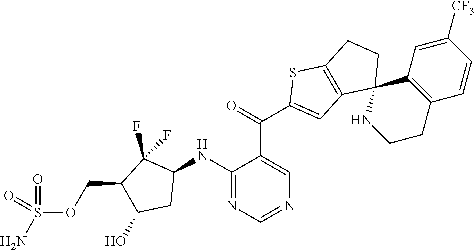 | 660 |
| 898 | 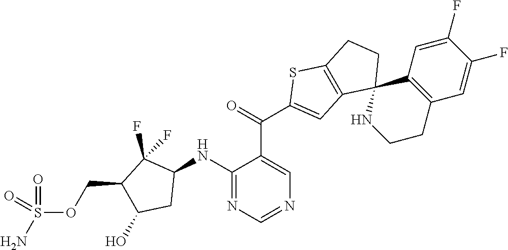 | 628 |
| 899 | 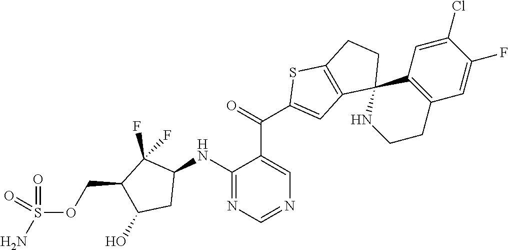 | 644 |
| 900 | 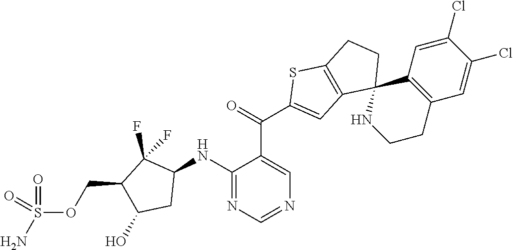 | 660 |
| 901 | 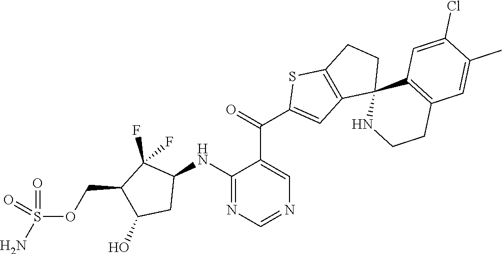 | 640 |
| 902 | 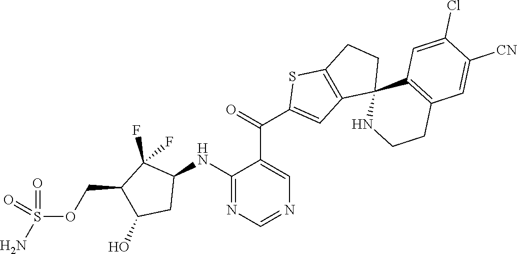 | 651 |
| 903 | 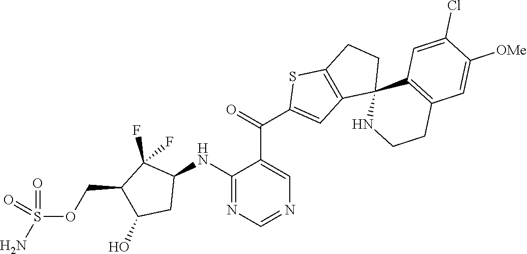 | 656 |
| 904 | 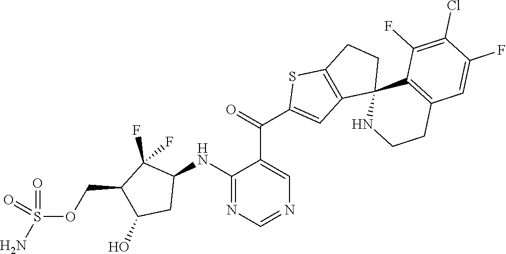 | 662 |
| 905 | 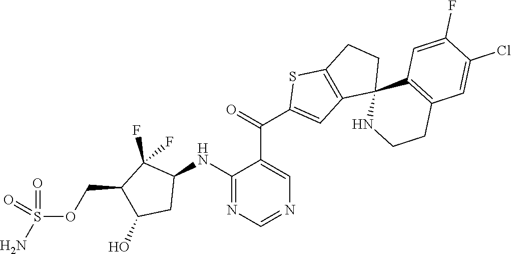 | 644 |
| 906 | 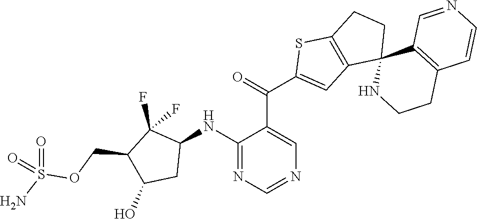 | 593 |
| 907 | 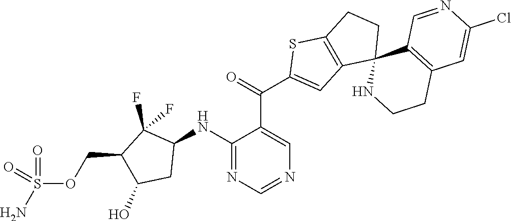 | 627 |
| 908 | 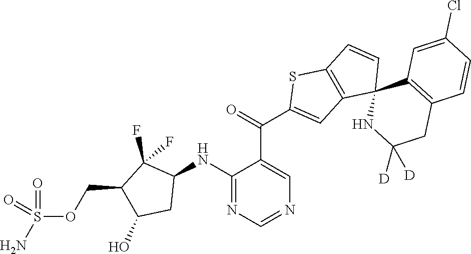 | 626 |
| 909 | 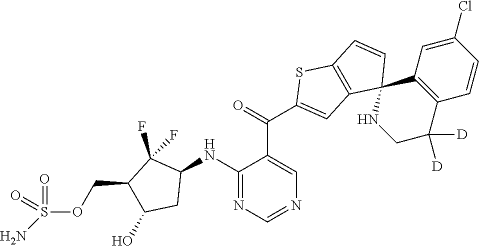 | 626 |
| 910 | 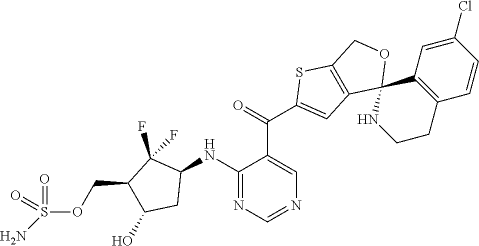 | 628 |
| 911 | 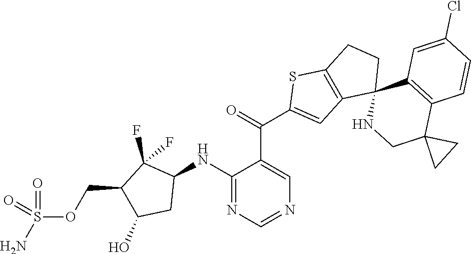 | 652 |
| 912 | 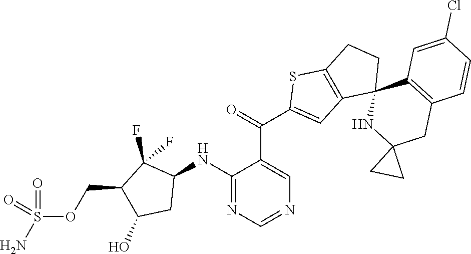 | 652 |
| 913 | 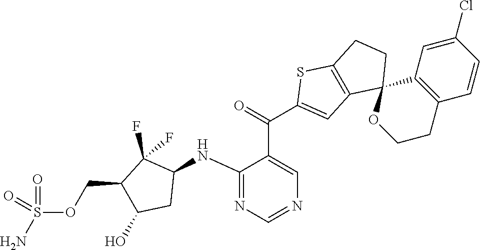 | 627 |
| 914 | 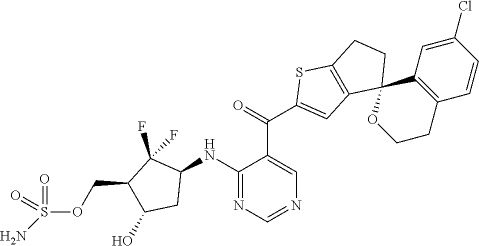 | 627 |
| 915 | 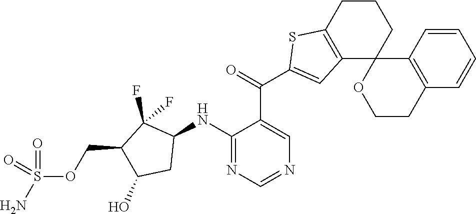 | 607 |
| 916 | 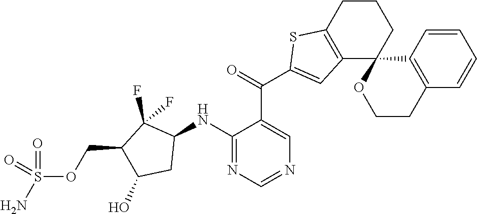 | 607 |
| 917 | 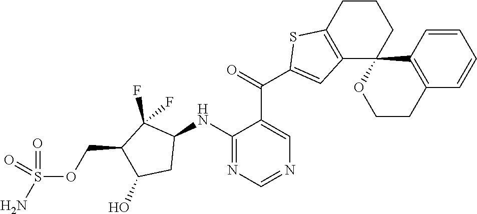 | 607 |
| 918 | 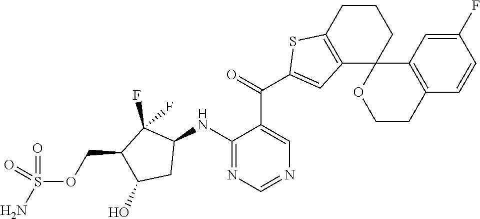 | 625 |
| 919 | 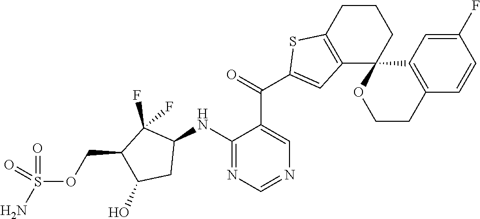 | 625 |
| 920 | 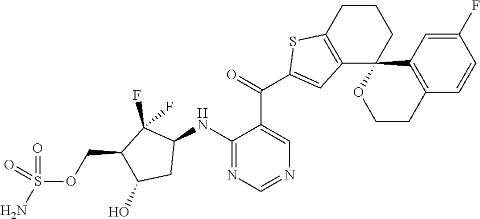 | 625 |
| 921 | 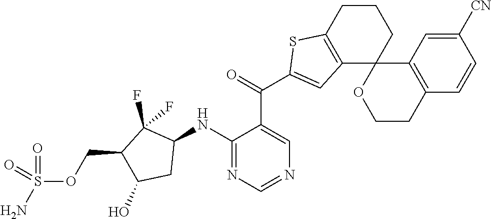 | 632 |
| 922 | 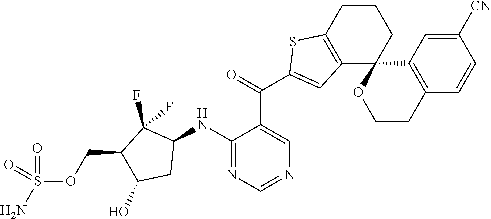 | 632 |
| 923 | 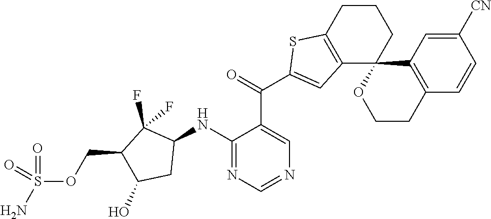 | 632 |
| 924 | 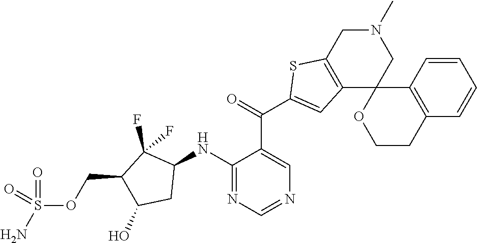 | 622 |
| 925 | 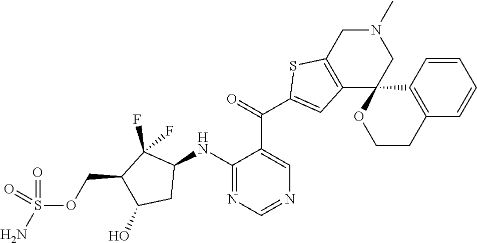 | 622 |
| 926 | 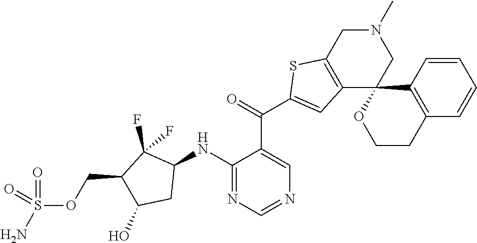 | 622 |
| 927 | 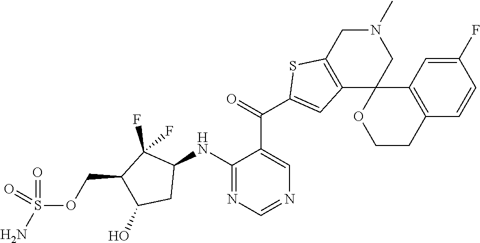 | 640 |
| 928 | 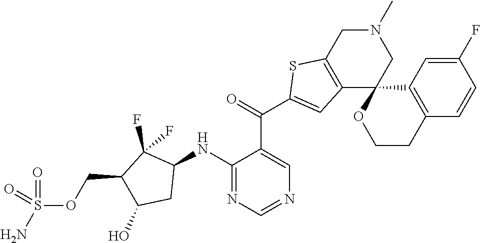 | 640 |
| 929 | 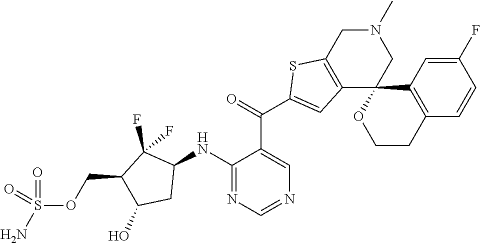 | 640 |
| 930 | 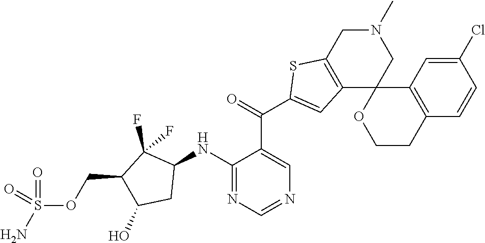 | 656 |
| 931 | 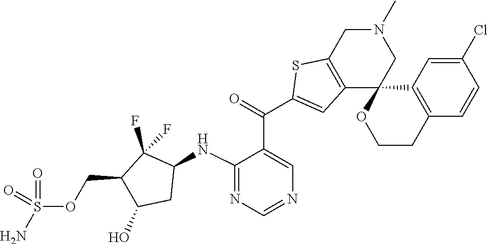 | 656 |
| 932 | 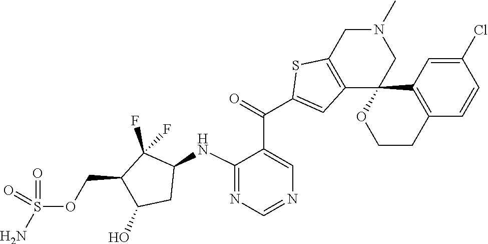 | 656 |
| 933 | 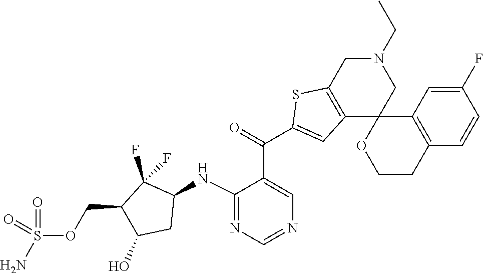 | 654 |
| 934 | 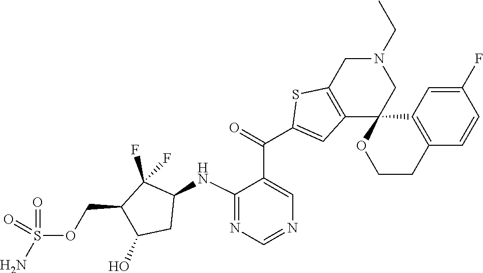 | 654 |
| 935 | 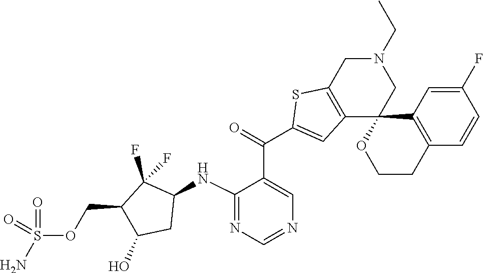 | 654 |
| 936 | 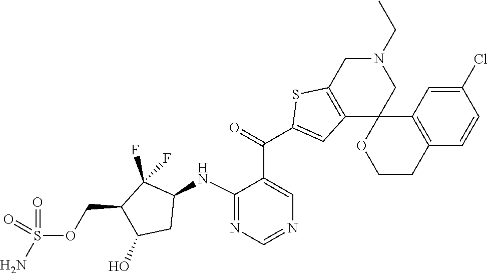 | 670 |
| 937 | 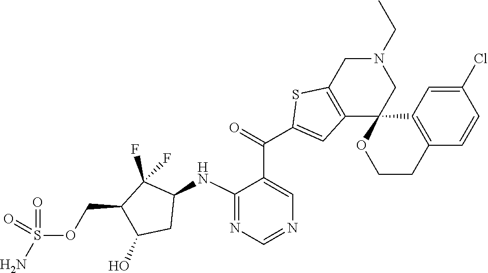 | 670 |
| 938 | 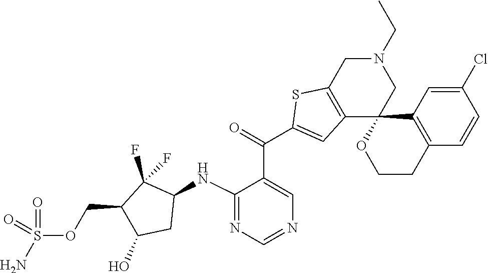 | 670 |
| 939 | 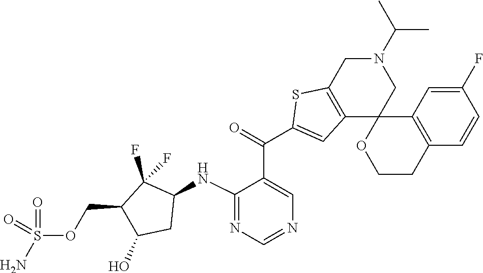 | 668 |
| 940 | 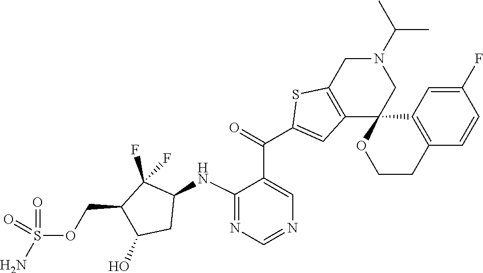 | 668 |
| 941 | 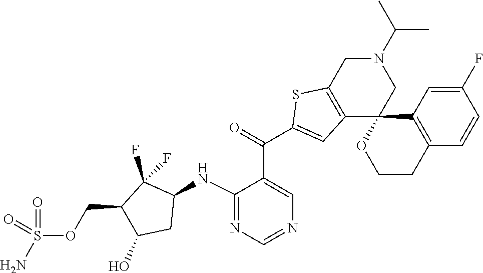 | 668 |
| 942 | 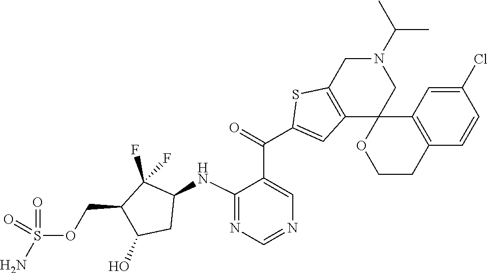 | 684 |
| 943 | 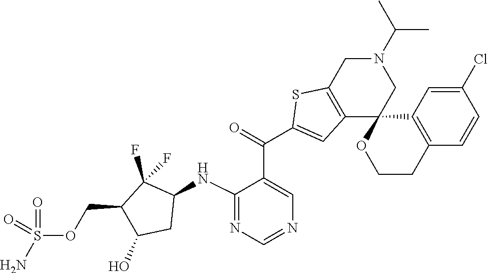 | 684 |
| 944 | 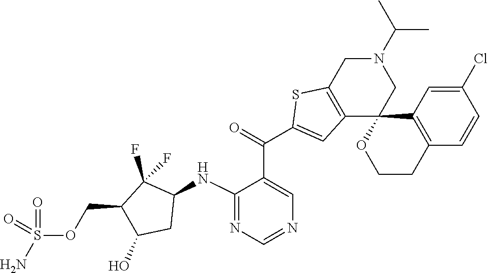 | 684 |
| 945 | 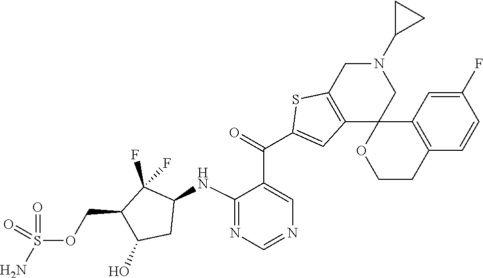 | 666 |
| 946 | 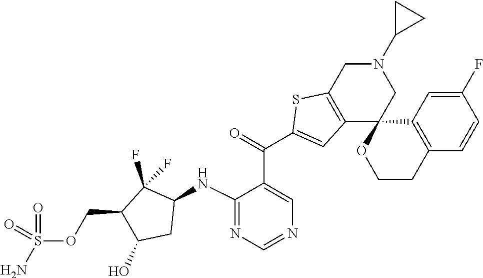 | 666 |
| 947 | 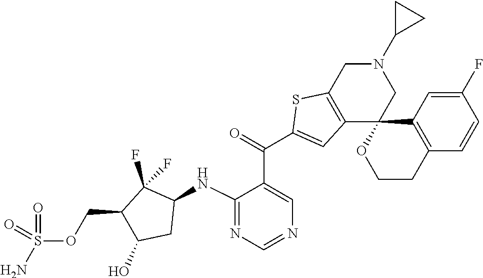 | 666 |
| 948 | 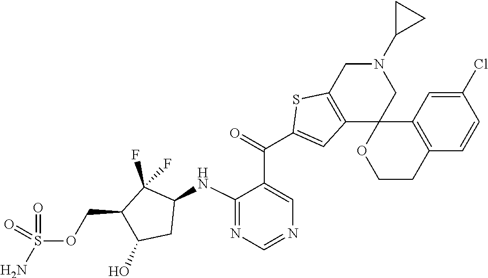 | 682 |
| 949 | 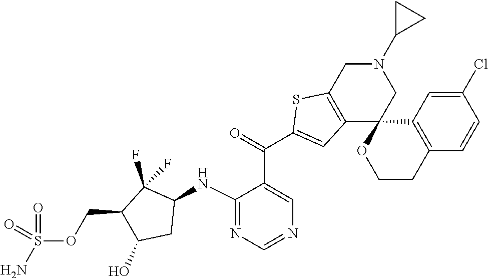 | 682 |
| 950 | 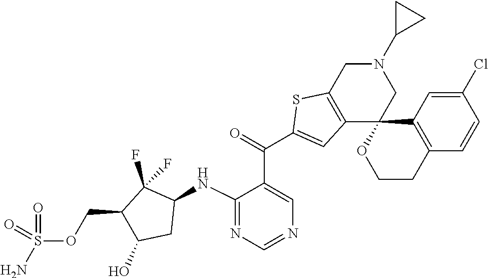 | 682 |
| 951 | 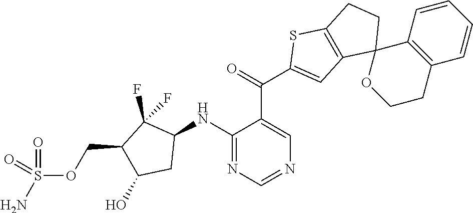 | 593 |
| 952 | 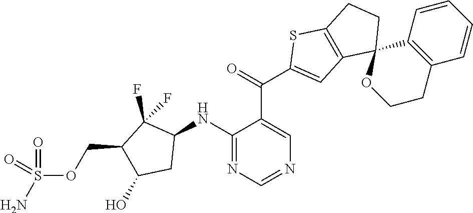 | 593 |
| 953 | 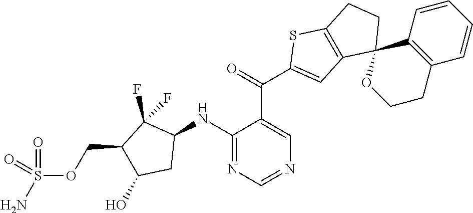 | 593 |
| 954 | 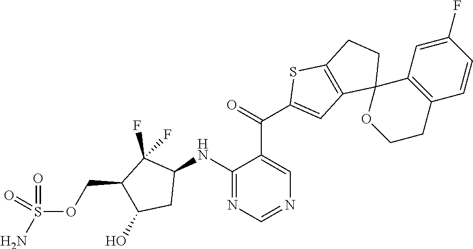 | 611 |
| 955 | 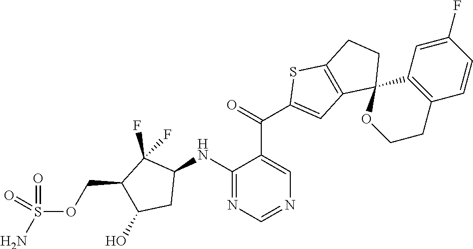 | 611 |
| 956 | 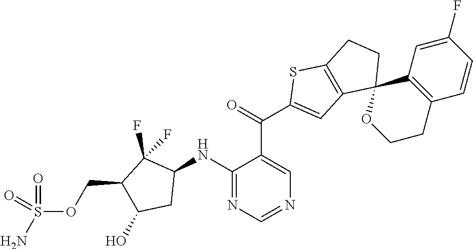 | 611 |
| 957 | 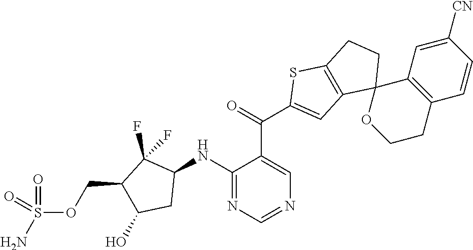 | 618 |
| 958 | 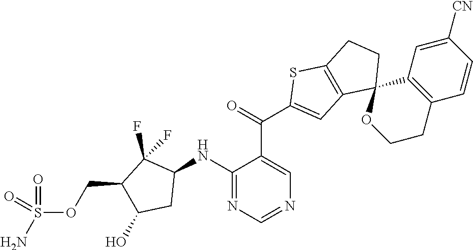 | 618 |
| 959 | 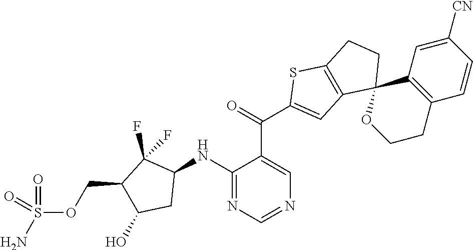 | 618 |
| 960 | 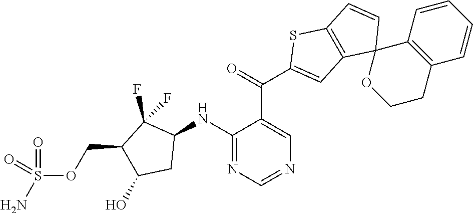 | 591 |
| 961 | 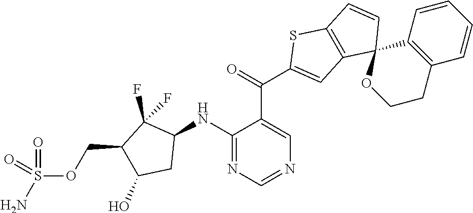 | 591 |
| 962 | 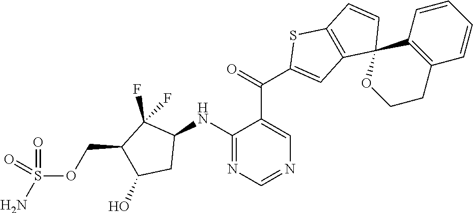 | 591 |
| 963 | 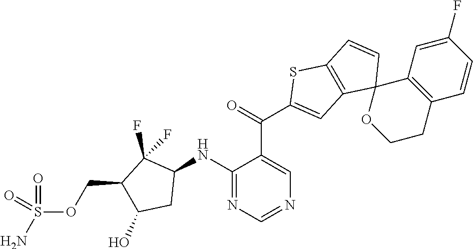 | 609 |
| 964 | 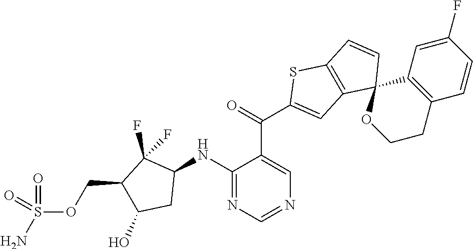 | 609 |
| 965 | 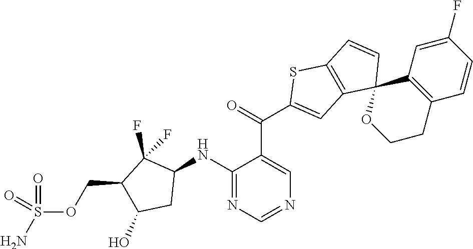 | 609 |
| 966 | 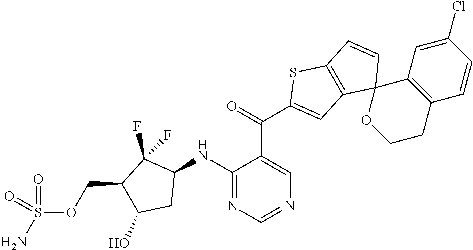 | 625 |
| 967 | 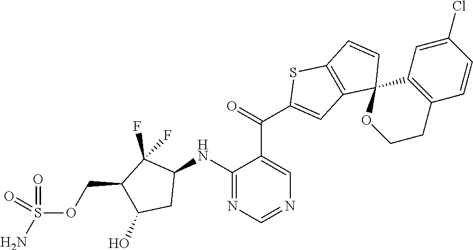 | 625 |
| 968 | 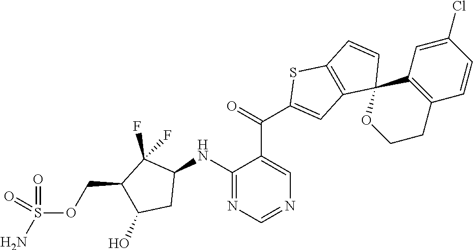 | 625 |
| 969 | 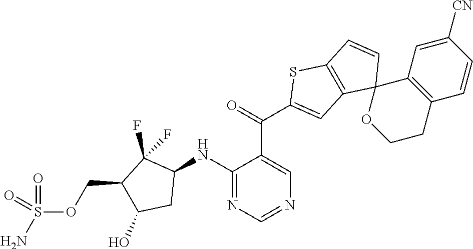 | 616 |
| 970 | 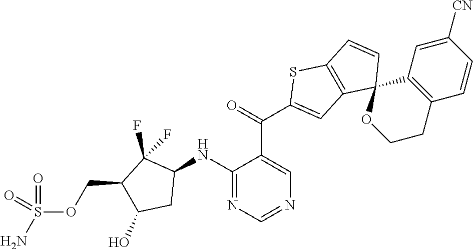 | 616 |
| 971 | 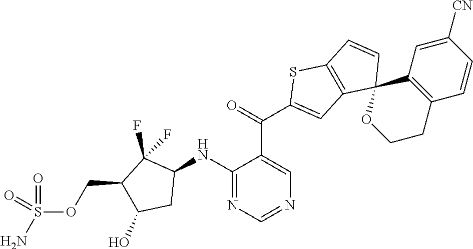 | 616 |
| 972 | 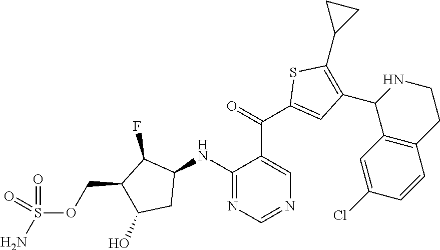 [SMILES: NS(=O)(=O)OC[C@H]1[C@@H](F)[C@@H](Nc2ncncc2C(=O)c2cc(C3NCCc4ccc(Cl)cc43)c(C3CC3)s2)C[C@@H]1O] | 622 |
| 973 | 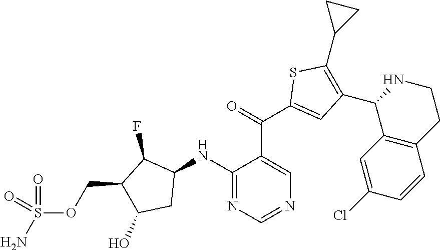 [SMILES: NS(=O)(=O)OC[C@H]1[C@@H](F)[C@@H](Nc2ncncc2C(=O)c2cc([C@@H]3NCCc4ccc(Cl)cc43)c(C3CC3)s2)C[C@@H]1O] | 622 |
| 974 | 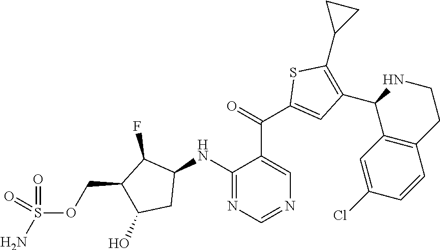 [SMILES: NS(=O)(=O)OC[C@H]1[C@@H](F)[C@@H](Nc2ncncc2C(=O)c2cc([C@H]3NCCc4ccc(Cl)cc43)c(C3CC3)s2)C[C@@H]1O] | 622 |
| 975 | 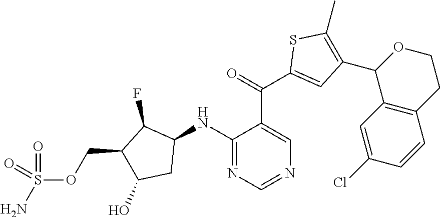 [SMILES: Cc1sc(C(=O)c2cncnc2N[C@H]2C[C@H](O)[C@@H](COS(N)(=O)=O)[C@H]2F)cc1C1OCCc2ccc(Cl)cc21] | 597 |
| 976 | 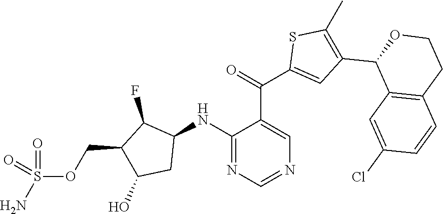 [SMILES: Cc1sc(C(=O)c2cncnc2N[C@H]2C[C@H](O)[C@@H](COS(N)(=O)=O)[C@H]2F)cc1[C@@H]1OCCc2ccc(Cl)cc21] | 597 |
| 977 | 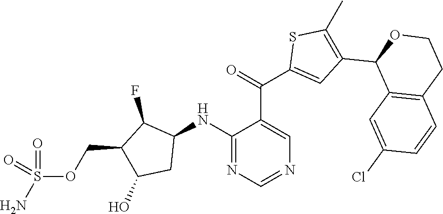 [SMILES: Cc1sc(C(=O)c2cncnc2N[C@H]2C[C@H](O)[C@@H](COS(N)(=O)=O)[C@H]2F)cc1[C@H]1OCCc2ccc(Cl)cc21] | 597 |
| 978 | 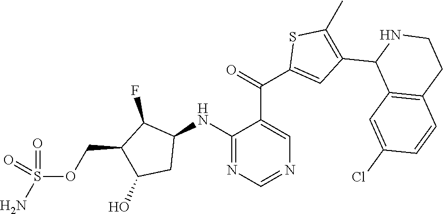 [SMILES: Cc1sc(C(=O)c2cncnc2N[C@H]2C[C@H](O)[C@@H](COS(N)(=O)=O)[C@H]2F)cc1C1NCCc2ccc(Cl)cc21] | 594 |
| 979 | 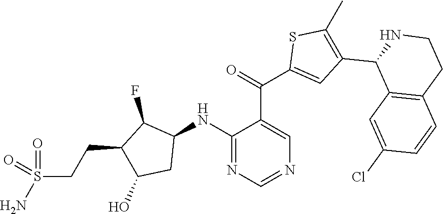 [SMILES: Cc1sc(C(=O)c2cncnc2N[C@H]2C[C@H](O)[C@@H](CCS(N)(=O)=O)[C@H]2F)cc1[C@@H]1NCCc2ccc(Cl)cc21] | 594 |
| 980 | 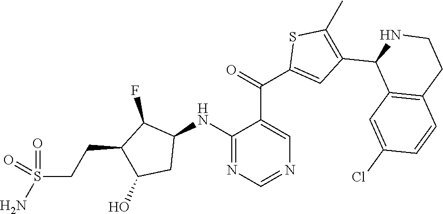 [SMILES: Cc1sc(C(=O)c2cncnc2N[C@H]2C[C@H](O)[C@@H](CCS(N)(=O)=O)[C@H]2F)cc1[C@H]1NCCc2ccc(Cl)cc21] | 594 |
| 981 | 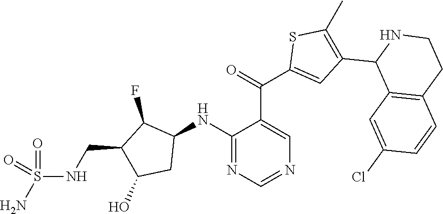 [SMILES: Cc1sc(C(=O)c2cncnc2N[C@H]2C[C@H](O)[C@@H](CNS(N)(=O)=O)[C@H]2F)cc1C1NCCc2ccc(Cl)cc21] | 595 |
| 982 | 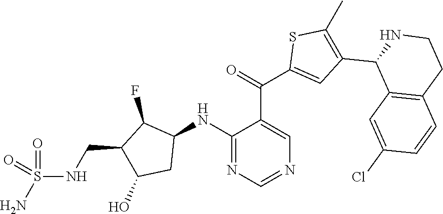 [SMILES: Cc1sc(C(=O)c2cncnc2N[C@H]2C[C@H](O)[C@@H](CNS(N)(=O)=O)[C@H]2F)cc1[C@@H]1NCCc2ccc(Cl)cc21] | 595 |
| 983 | 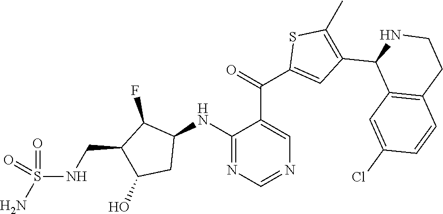 [SMILES: Cc1sc(C(=O)c2cncnc2N[C@H]2C[C@H](O)[C@@H](CNS(N)(=O)=O)[C@H]2F)cc1[C@H]1NCCc2ccc(Cl)cc21] | 595 |
| 984 | 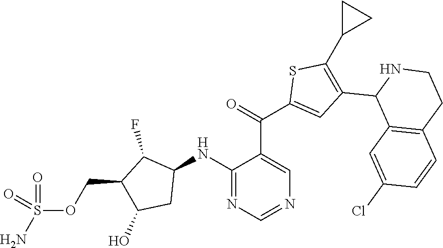 [SMILES: NS(=O)(=O)OC[C@H]1[C@H](F)[C@@H](Nc2ncncc2C(=O)c2cc(C3NCCc4ccc(Cl)cc43)c(C3CC3)s2)C[C@@H]1O] | 622 |
| 985 | 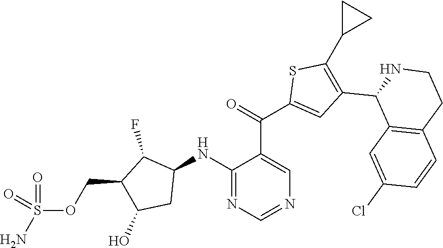 [SMILES: NS(=O)(=O)OC[C@H]1[C@H](F)[C@@H](Nc2ncncc2C(=O)c2cc([C@@H]3NCCc4ccc(Cl)cc43)c(C3CC3)s2)C[C@@H]1O] | 622 |
| 986 | 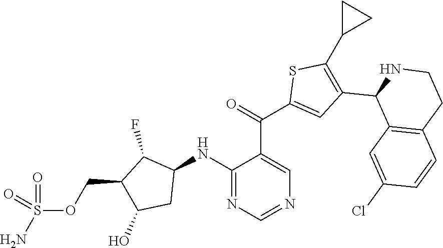 [SMILES: NS(=O)(=O)OC[C@H]1[C@H](F)[C@@H](Nc2ncncc2C(=O)c2cc([C@H]3NCCc4ccc(Cl)cc43)c(C3CC3)s2)C[C@@H]1O] | 622 |
| 987 | 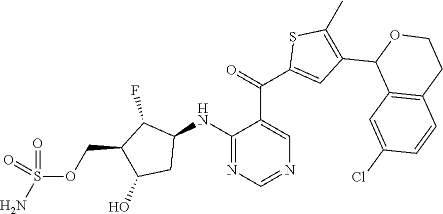 [SMILES: Cc1sc(C(=O)c2cncnc2N[C@H]2C[C@H](O)[C@@H](COS(N)(=O)=O)[C@@H]2F)cc1C1OCCc2ccc(Cl)cc21] | 597 |
| 988 | 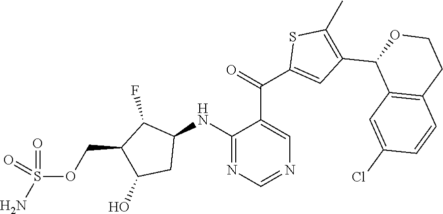 [SMILES: Cc1sc(C(=O)c2cncnc2N[C@H]2C[C@H](O)[C@@H](COS(N)(=O)=O)[C@@H]2F)cc1[C@@H]1OCCc2ccc(Cl)cc21] | 597 |
| 989 | 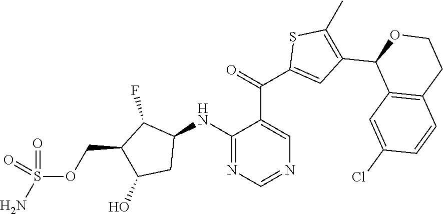 [SMILES: Cc1sc(C(=O)c2cncnc2N[C@H]2C[C@H](O)[C@@H](COS(N)(=O)=O)[C@@H]2F)cc1[C@H]1OCCc2ccc(Cl)cc21] | 597 |
| 990 | 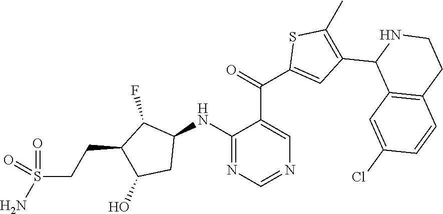 [SMILES: Cc1sc(C(=O)c2cncnc2N[C@H]2C[C@H](O)[C@@H](CCS(N)(=O)=O)[C@@H]2F)cc1C1NCCc2ccc(Cl)cc21] | 594 |
| 991 | 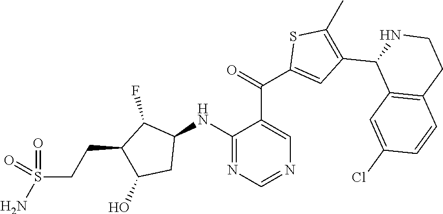 [SMILES: Cc1sc(C(=O)c2cncnc2N[C@H]2C[C@H](O)[C@@H](CCS(N)(=O)=O)[C@@H]2F)cc1[C@@H]1NCCc2ccc(Cl)cc21] | 594 |
| 992 | 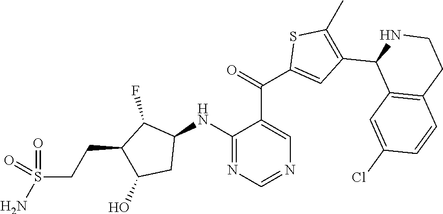 [SMILES: Cc1sc(C(=O)c2cncnc2N[C@H]2C[C@H](O)[C@@H](CCS(N)(=O)=O)[C@@H]2F)cc1[C@H]1NCCc2ccc(Cl)cc21] | 594 |
| 993 | 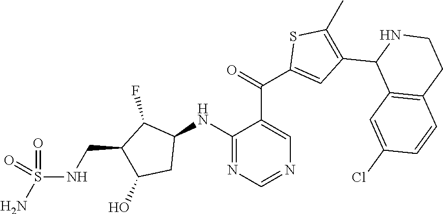 [SMILES: Cc1sc(C(=O)c2cncnc2N[C@H]2C[C@H](O)[C@@H](CNS(N)(=O)=O)[C@@H]2F)cc1C1NCCc2ccc(Cl)cc21] | 595 |
| 994 | 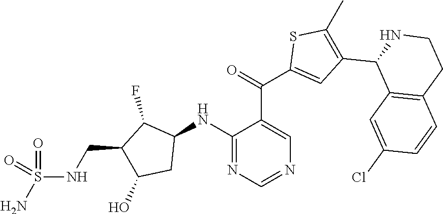 [SMILES: Cc1sc(C(=O)c2cncnc2N[C@H]2C[C@H](O)[C@@H](CNS(N)(=O)=O)[C@@H]2F)cc1[C@@H]1NCCc2ccc(Cl)cc21] | 595 |
| 995 | 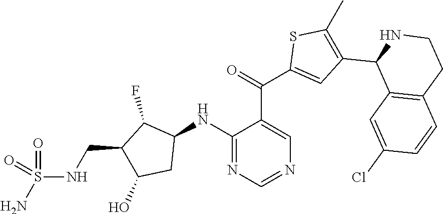 [SMILES: Cc1sc(C(=O)c2cncnc2N[C@H]2C[C@H](O)[C@@H](CNS(N)(=O)=O)[C@@H]2F)cc1[C@H]1NCCc2ccc(Cl)cc21] | 595 |
| 996 | 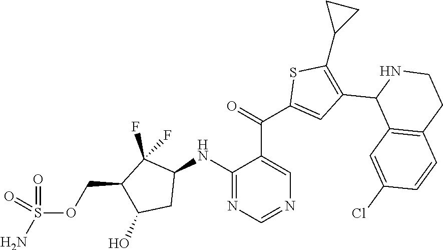 | 640 |
| 997 | 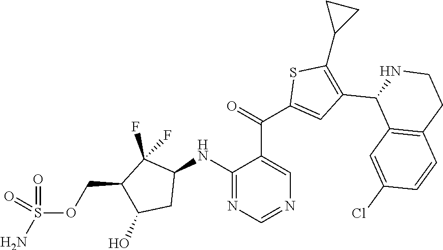 | 640 |
| 998 | 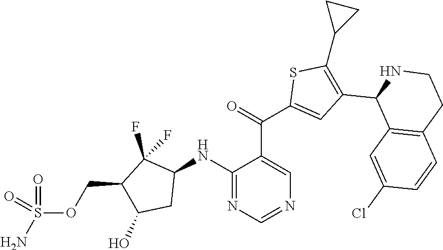 | 640 |
| 999 | 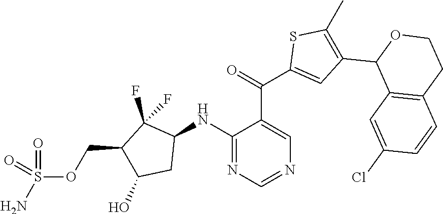 | 615 |
| 1000 | 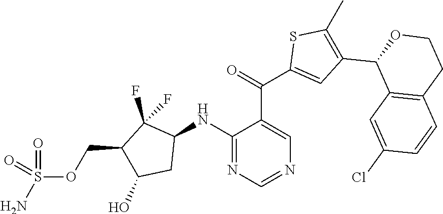 | 615 |
| 1001 | 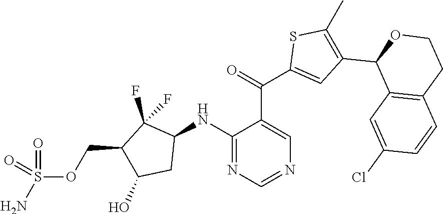 | 615 |
| 1002 | 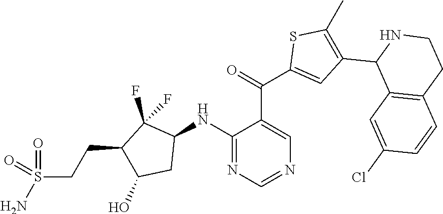 | 612 |
| 1003 | 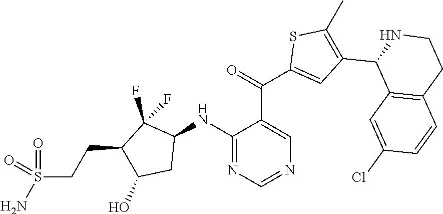 | 612 |
| 1004 | 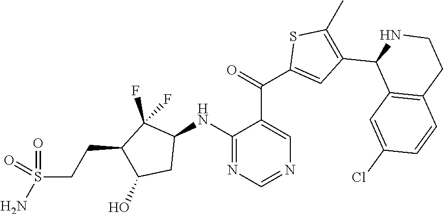 | 612 |
| 1005 | 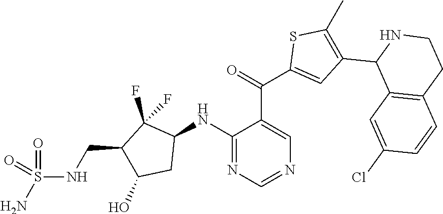 | 613 |
| 1006 | 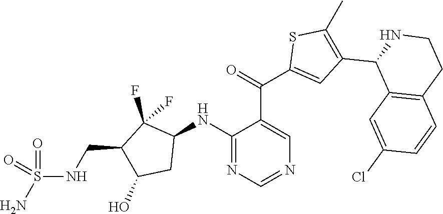 | 613 |
| 1007 | 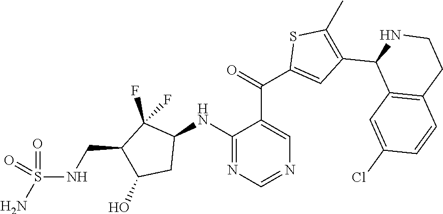 | 613 |
| 1008 | 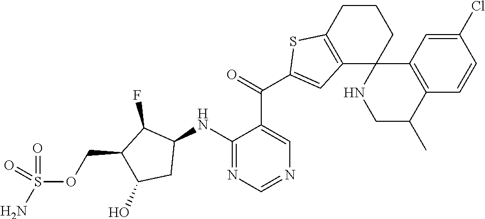 [SMILES: CC1CNC2(CCCc3sc(C(=O)c4cncnc4N[C@H]4C[C@H](O)[C@@H](COS(N)(=O)=O)[C@H]4F)cc32)c2cc(Cl)ccc21] | 636 |
| 1009 | 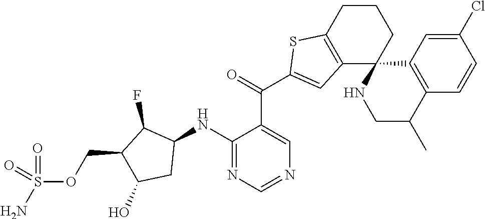 | 636 |
| 1010 | 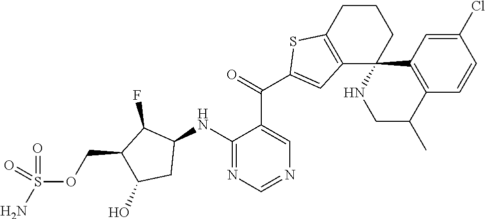 | 636 |
| 1011 | 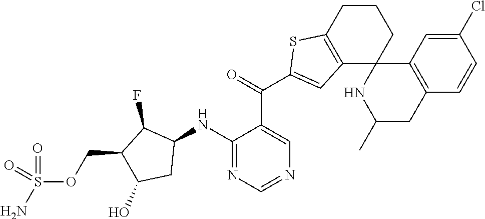 [SMILES: CC1Cc2ccc(Cl)cc2C2(CCCc3sc(C(=O)c4cncnc4N[C@H]4C[C@H](O)[C@@H](COS(N)(=O)=O)[C@H]4F)cc32)N1] | 636 |
| 1012 | 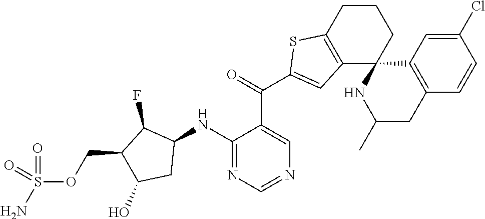 | 636 |
| 1013 | 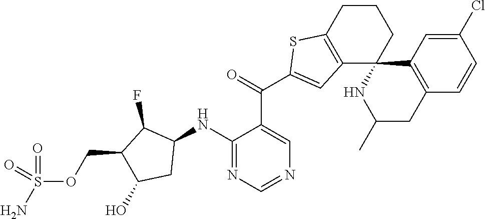 | 636 |
| 1014 | 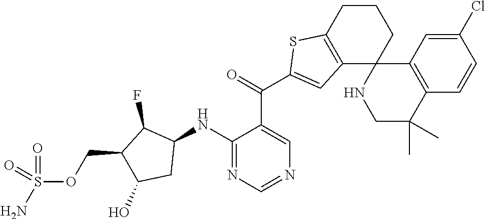 [SMILES: CC1(C)CNC2(CCCc3sc(C(=O)c4cncnc4N[C@H]4C[C@H](O)[C@@H](COS(N)(=O)=O)[C@H]4F)cc32)c2cc(Cl)ccc21] | 650 |
| 1015 | 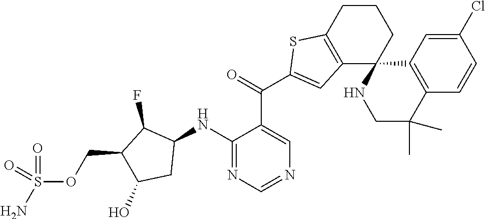 [SMILES: CC1(C)CN[C@@]2(CCCc3sc(C(=O)c4cncnc4N[C@H]4C[C@H](O)[C@@H](COS(N)(=O)=O)[C@H]4F)cc32)c2cc(Cl)ccc21] | 650 |
| 1016 | 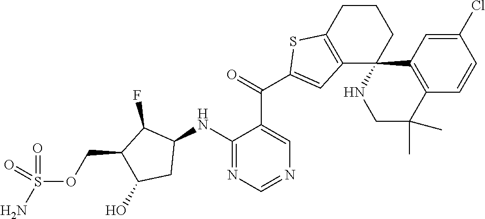 [SMILES: CC1(C)CN[C@]2(CCCc3sc(C(=O)c4cncnc4N[C@H]4C[C@H](O)[C@@H](COS(N)(=O)=O)[C@H]4F)cc32)c2cc(Cl)ccc21] | 650 |
| 1017 | 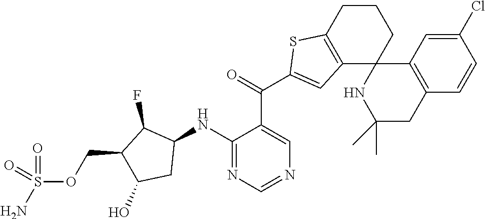 [SMILES: CC1(C)Cc2ccc(Cl)cc2C2(CCCc3sc(C(=O)c4cncnc4N[C@H]4C[C@H](O)[C@@H](COS(N)(=O)=O)[C@H]4F)cc32)N1] | 650 |
| 1018 | 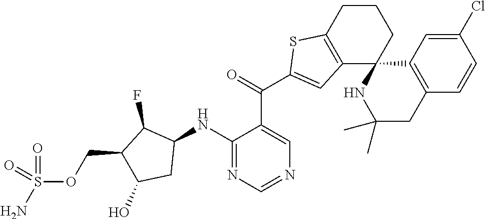 [SMILES: CC1(C)Cc2ccc(Cl)cc2[C@]2(CCCc3sc(C(=O)c4cncnc4N[C@H]4C[C@H](O)[C@@H](COS(N)(=O)=O)[C@H]4F)cc32)N1] | 650 |
| 1019 | 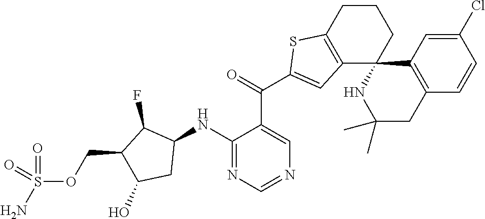 [SMILES: CC1(C)Cc2ccc(Cl)cc2[C@@]2(CCCc3sc(C(=O)c4cncnc4N[C@H]4C[C@H](O)[C@@H](COS(N)(=O)=O)[C@H]4F)cc32)N1] | 650 |
| 1020 | 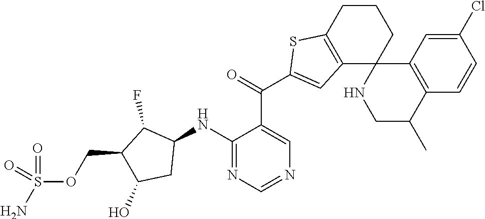 [SMILES: CC1CNC2(CCCc3sc(C(=O)c4cncnc4N[C@H]4C[C@H](O)[C@@H](COS(N)(=O)=O)[C@@H]4F)cc32)c2cc(Cl)ccc21] | 636 |
| 1021 | 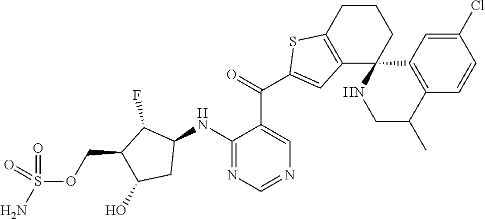 | 636 |
| 1022 | 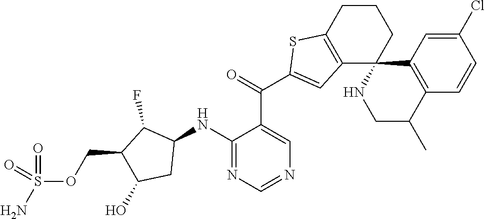 | 636 |
| 1023 | 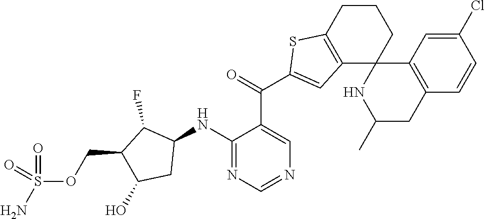 [SMILES: CC1Cc2ccc(Cl)cc2C2(CCCc3sc(C(=O)c4cncnc4N[C@H]4C[C@H](O)[C@@H](COS(N)(=O)=O)[C@@H]4F)cc32)N1] | 636 |
| 1024 | 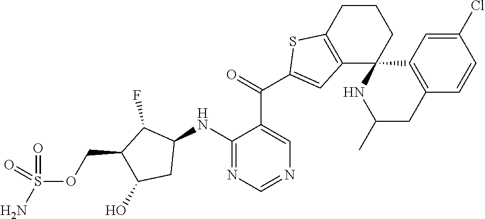 | 636 |
| 1025 | 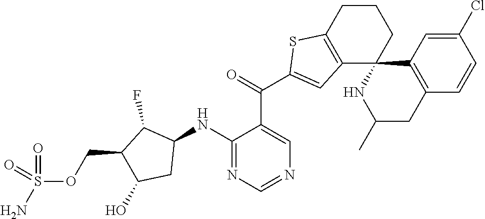 | 636 |
| 1026 | 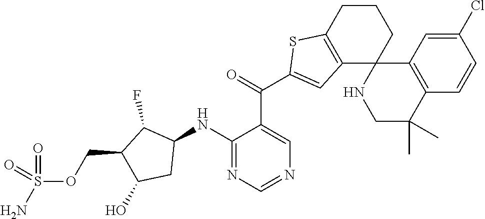 [SMILES: CC1(C)CNC2(CCCc3sc(C(=O)c4cncnc4N[C@H]4C[C@H](O)[C@@H](COS(N)(=O)=O)[C@@H]4F)cc32)c2cc(Cl)ccc21] | 650 |
| 1027 | 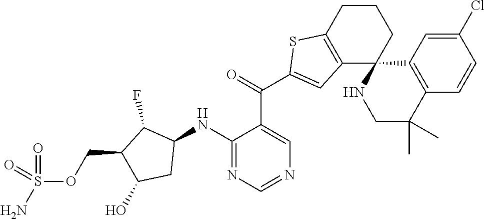 [SMILES: CC1(C)CN[C@@]2(CCCc3sc(C(=O)c4cncnc4N[C@H]4C[C@H](O)[C@@H](COS(N)(=O)=O)[C@@H]4F)cc32)c2cc(Cl)ccc21] | 650 |
| 1028 | 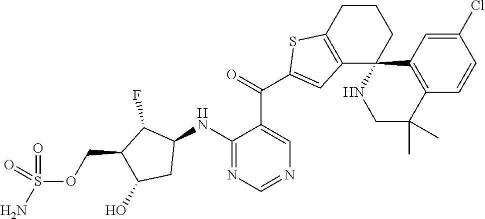 [SMILES: CC1(C)CN[C@]2(CCCc3sc(C(=O)c4cncnc4N[C@H]4C[C@H](O)[C@@H](COS(N)(=O)=O)[C@@H]4F)cc32)c2cc(Cl)ccc21] | 650 |
| 1029 | 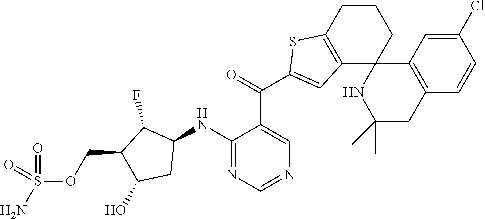 [SMILES: CC1(C)Cc2ccc(Cl)cc2C2(CCCc3sc(C(=O)c4cncnc4N[C@H]4C[C@H](O)[C@@H](COS(N)(=O)=O)[C@@H]4F)cc32)N1] | 650 |
| 1030 | 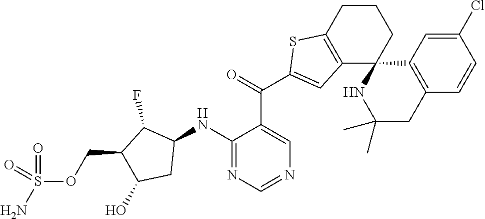 [SMILES: CC1(C)Cc2ccc(Cl)cc2[C@]2(CCCc3sc(C(=O)c4cncnc4N[C@H]4C[C@H](O)[C@@H](COS(N)(=O)=O)[C@@H]4F)cc32)N1] | 650 |
| 1031 | 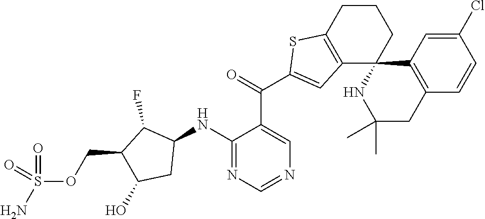 [SMILES: CC1(C)Cc2ccc(Cl)cc2[C@@]2(CCCc3sc(C(=O)c4cncnc4N[C@H]4C[C@H](O)[C@@H](COS(N)(=O)=O)[C@@H]4F)cc32)N1] | 650 |
| 1032 | 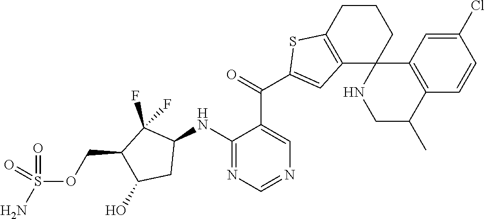 | 654 |
| 1033 | 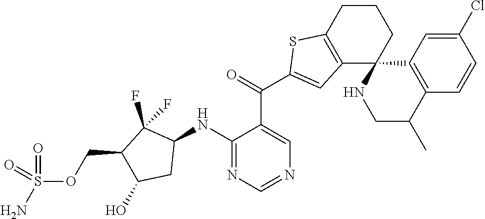 | 654 |
| 1034 | 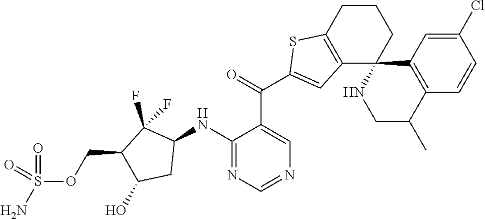 | 654 |
| 1035 | 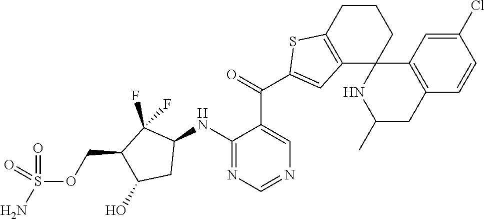 | 654 |
| 1036 | 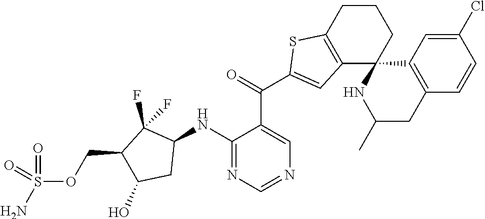 | 654 |
| 1037 | 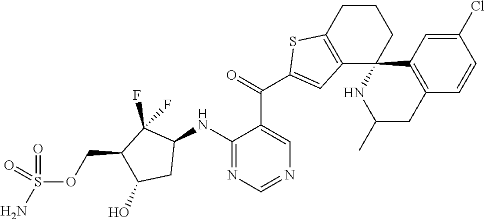 | 654 |
| 1038 | 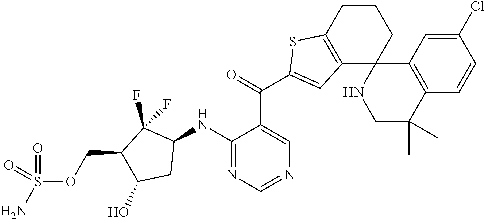 | 668 |
| 1039 | 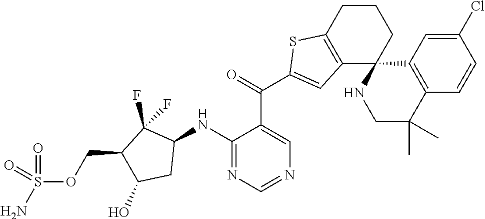 | 668 |
| 1040 | 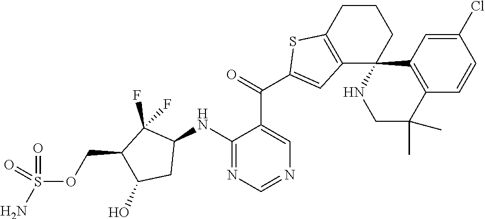 | 668 |
| 1041 | 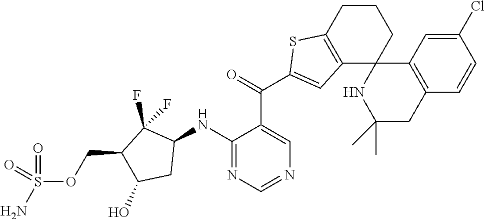 | 668 |
| 1042 | 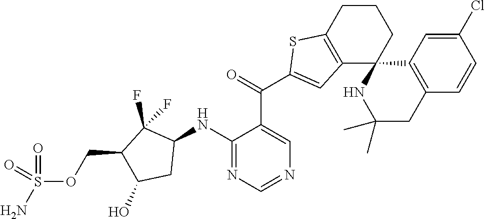 | 668 |
| 1043 | 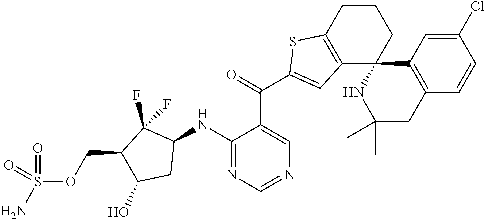 | 668 |
| 1044 | 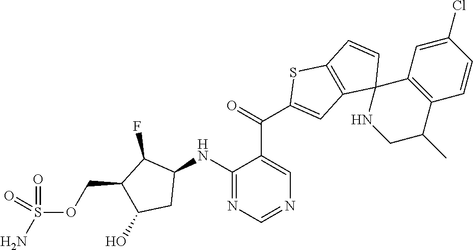 [SMILES: CC1CNC2(C=Cc3sc(C(=O)c4cncnc4N[C@H]4C[C@H](O)[C@@H](COS(N)(=O)=O)[C@H]4F)cc32)c2cc(Cl)ccc21] | 620 |
| 1045 | 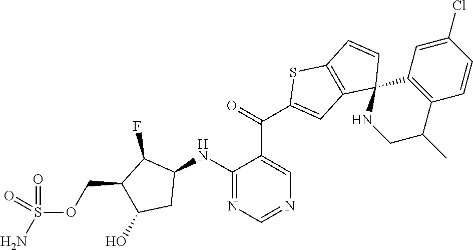 | 620 |
| 1046 | 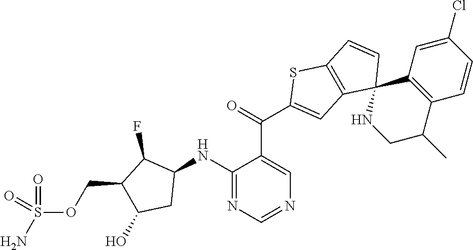 | 620 |
| 1047 | 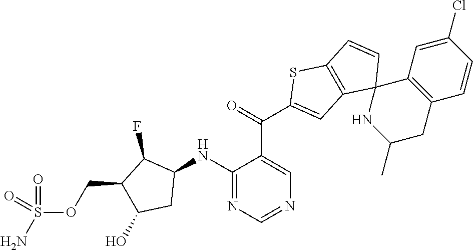 [SMILES: CC1Cc2ccc(Cl)cc2C2(C=Cc3sc(C(=O)c4cncnc4N[C@H]4C[C@H](O)[C@@H](COS(N)(=O)=O)[C@H]4F)cc32)N1] | 620 |
| 1048 | 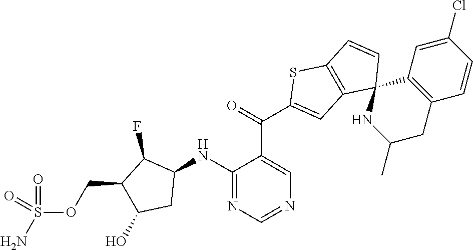 | 620 |
| 1049 | 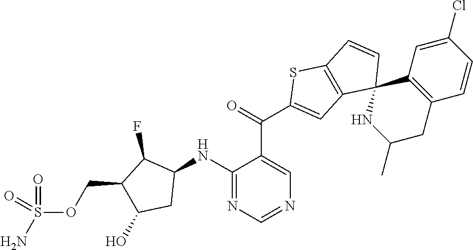 | 620 |
| 1050 | 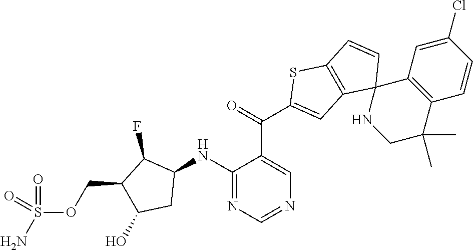 [SMILES: CC1(C)CNC2(C=Cc3sc(C(=O)c4cncnc4N[C@H]4C[C@H](O)[C@@H](COS(N)(=O)=O)[C@H]4F)cc32)c2cc(Cl)ccc21] | 634 |
| 1051 | 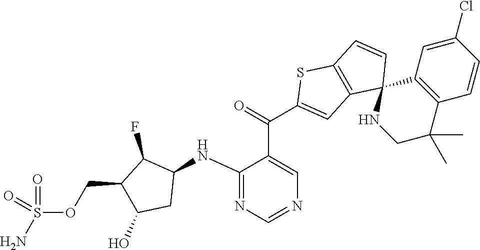 [SMILES: CC1(C)CN[C@@]2(C=Cc3sc(C(=O)c4cncnc4N[C@H]4C[C@H](O)[C@@H](COS(N)(=O)=O)[C@H]4F)cc32)c2cc(Cl)ccc21] | 634 |
| 1052 | 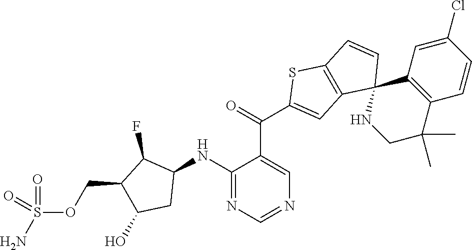 [SMILES: CC1(C)CN[C@]2(C=Cc3sc(C(=O)c4cncnc4N[C@H]4C[C@H](O)[C@@H](COS(N)(=O)=O)[C@H]4F)cc32)c2cc(Cl)ccc21] | 634 |
| 1053 | 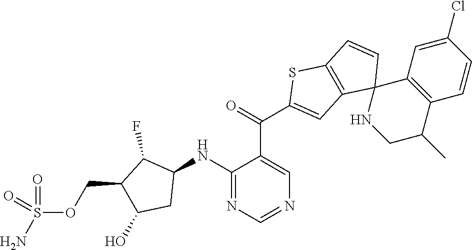 [SMILES: CC1CNC2(C=Cc3sc(C(=O)c4cncnc4N[C@H]4C[C@H](O)[C@@H](COS(N)(=O)=O)[C@@H]4F)cc32)c2cc(Cl)ccc21] | 620 |
| 1054 | 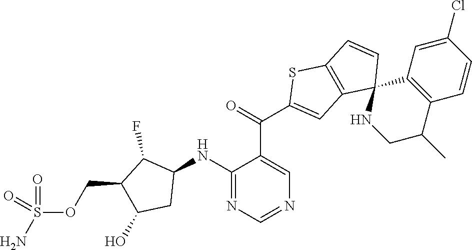 | 620 |
| 1055 | 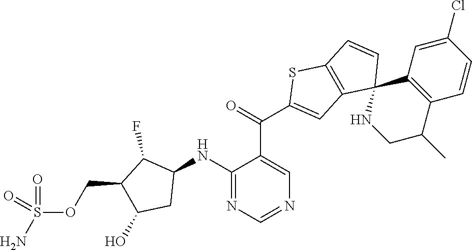 | 620 |
| 1056 | 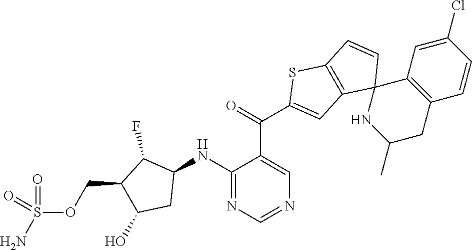 [SMILES: CC1Cc2ccc(Cl)cc2C2(C=Cc3sc(C(=O)c4cncnc4N[C@H]4C[C@H](O)[C@@H](COS(N)(=O)=O)[C@@H]4F)cc32)N1] | 620 |
| 1057 | 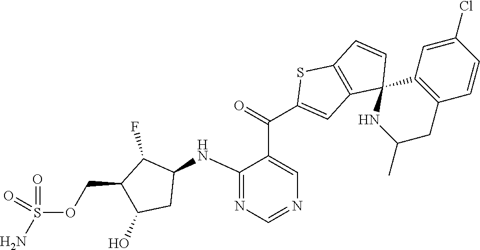 | 620 |
| 1058 | 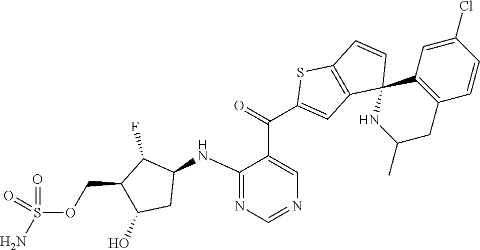 | 620 |
| 1059 | 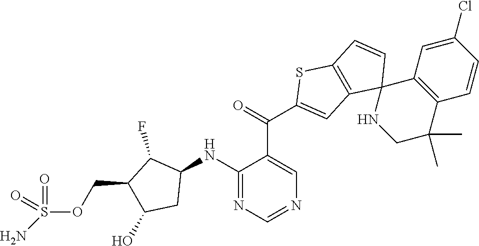 [SMILES: CC1(C)CNC2(C=Cc3sc(C(=O)c4cncnc4N[C@H]4C[C@H](O)[C@@H](COS(N)(=O)=O)[C@@H]4F)cc32)c2cc(Cl)ccc21] | 634 |
| 1060 | 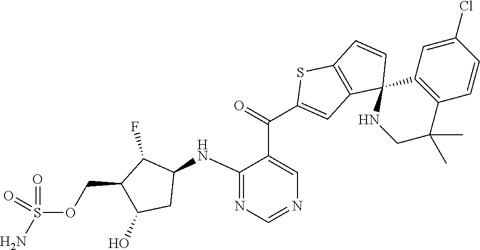 [SMILES: CC1(C)CN[C@@]2(C=Cc3sc(C(=O)c4cncnc4N[C@H]4C[C@H](O)[C@@H](COS(N)(=O)=O)[C@@H]4F)cc32)c2cc(Cl)ccc21] | 634 |
| 1061 | 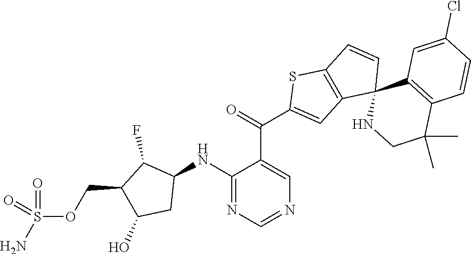 [SMILES: CC1(C)CN[C@]2(C=Cc3sc(C(=O)c4cncnc4N[C@H]4C[C@H](O)[C@@H](COS(N)(=O)=O)[C@@H]4F)cc32)c2cc(Cl)ccc21] | 634 |
| 1062 | 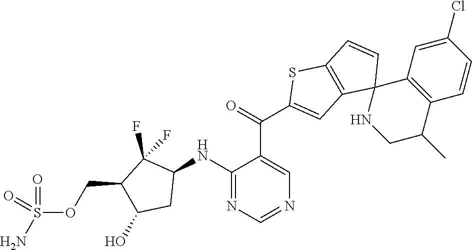 | 638 |
| 1063 | 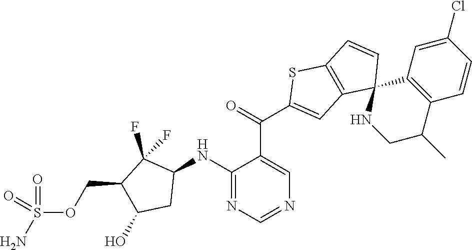 | 638 |
| 1064 | 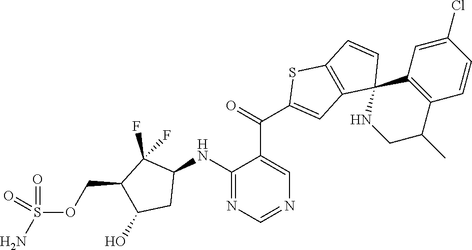 | 638 |
| 1065 | 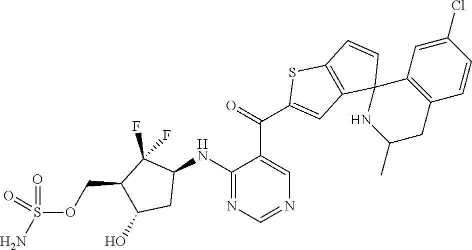 | 638 |
| 1066 | 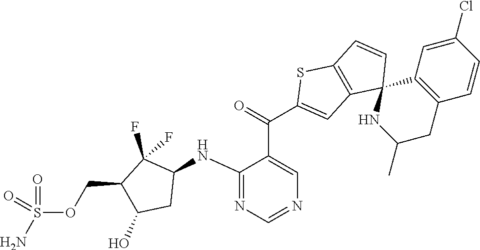 | 638 |
| 1067 | 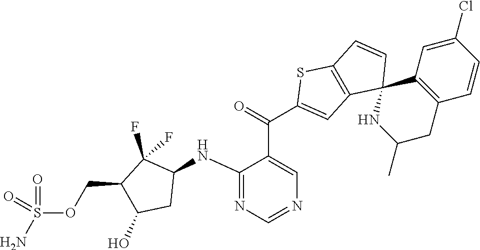 | 638 |
| 1068 | 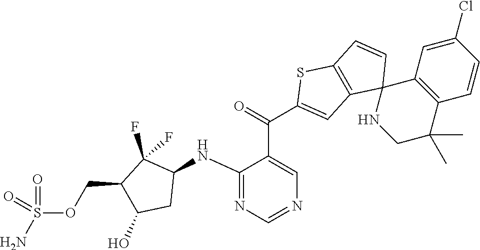 | 652 |
| 1069 | 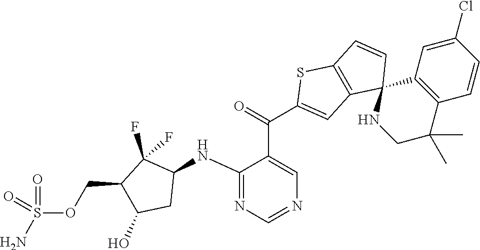 | 652 |
| 1070 | 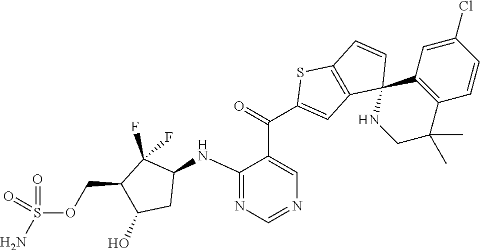 | 652 |

### Biological Example 1. In Vitro Inhibitory Activity Assay of Compounds of the Present Disclosure Against SAE

HCT116 cells were seeded into a 96-well black transparent bottom plate at about 20000/well with 90 μL per well. The cells were incubated at 37° C. overnight for 24 h. The test compounds were 10-fold diluted to give a final concentration, and 10 μL of the diluted compounds was added to each well of the cell culture plate. The mixture was incubated at 37° C. for 6 h. The cells were then washed gently, the culture medium was discarded, and the cells were washed once with 200 μL of 0.1% PBST. 50 μL of 4% PFA was added to each well, and the cells were immobilized at room temperature for 20 min, and then washed with PBS 2-3 times. 50 μL of 0.2% Triton X-100 was added to each well, and the cells were left to stand at room temperature for 15 min and washed with 0.1% PBST 3 times. 100 μL of 3% BSA (0.6 g BSA+20 mL PBS) was added to each well, and the mixture was blocked at 37° C. for 30 min. After the blocking was completed, the liquid was removed and a primary antibody (SUMO-2/3 (18H8) Rabbit mAb, 1:400 dilution) was prepared with 1% BSA. 30 μL of the diluted primary antibody was added to each well, and the mixture was incubated at 4° C. overnight. The cells were washed 3 times with 200 μL of 0.1% PBST. A FITC secondary antibody (Fluorescein (FITC)-conjugated Affinipure Goat Anti-Rabbit IgG (H+L), 1:300 dilution) was further prepared with 1% BSA, and 40 μL of the diluted secondary antibody was added to each well. The mixture was incubated at room temperature in the dark for 2 h, and then the cells were washed 4 times with 0.1% PBST. The fluorescence signal of the sample was detected and compared to the DMSO group, and the inhibition rate and IC₅₀ were calculated. The results are shown in Table 2 below.

TABLE 2
Inhibitory activity of the compounds of the present disclosure against SAE (IC₅₀, nM)

| Compound | SUMO IF IC₅₀ | Compound | SUMO IF IC₅₀ | Compound | SUMO IF IC₅₀ |
|---|---|---|---|---|---|
| 85 | ++ | 86 | + | 87 | +++ |
| 88 | +++ | 89 | >1000 | 90 | 21.1 |
| 91 | ++ | 92 | >1000 | 93 | 32.1 |
| 94 | ++ | 95 | + | 96 | +++ |
| 97 | + | 98 | + | 99 | ++ |
| 100 | ++ | 101 | + | 102 | ++ |
| 103 | ++ | 104 | >1000 | 105 | 79.3 |
| 106 | ++ | 107 | ++ | 108 | + |
| 109 | ++ | 110 | ++ | 111 | +++ |
| 112 | ++ | 113 | ++ | 114 | ++ |
| 115 | ++ | 116 | ++ | 117 | ++ |
| 118 | ++ | 119 | ++ | 120 | +++ |
| 121 | +++ | 122 | ++ | 123 | ++ |
| 124 | ++ | 125 | ++ | 126 | +++ |
| 127 | + | 128 | >1000 | 129 | 121.5 |
| 130 | ++ | 131 | >1000 | 132 | 40.6 |
| 133 | ++ | 134 | >1000 | 135 | 48.9 |
| 136 | ++ | 137 | + | 138 | ++ |
| 139 | + | 140 | + | 141 | ++ |
| 142 | + | 143 | + | 144 | ++ |
| 145 | ++ | 146 | + | 147 | ++ |
| 148 | ++ | 149 | ++ | 150 | + |
| 151 | ++ | 152 | ++ | 153 | +++ |
| 154 | ++ | 155 | ++ | 156 | ++ |
| 157 | ++ | 158 | ++ | 159 | ++ |
| 160 | ++ | 161 | ++ | 162 | +++ |
| 163 | ++ | 164 | ++ | 165 | ++ |
| 166 | ++ | 167 | ++ | 168 | +++ |
| 229 | + | 230 | ++ | 231 | + |
| 233 | 42.6 | 234 | >1000 | 345 | + |
| 235 | + | 236 | 73.1 | 237 | >1000 |
| 238 | + | 239 | ++ | 240 | + |
| 241 | ++ | 242 | ++ | 243 | + |
| 244 | ++ | 245 | ++ | 246 | ++ |
| 247 | ++ | 248 | ++ | 249 | ++ |
| 250 | ++ | 251 | ++ | 252 | ++ |
| 253 | ++ | 254 | ++ | 255 | ++ |
| 256 | + | 257 | + | 258 | ++ |
| 259 | ++ | 260 | ++ | 261 | ++ |
| 262 | ++ | 263 | ++ | 264 | ++ |
| 265 | ++ | 266 | ++ | 267 | ++ |
| 268 | ++ | 269 | ++ | 270 | ++ |
| 294 | + | 295 | + | 296 | ++ |
| 297 | +++ | 298 | + | 299 | +++ |
| 300 | +++ | 301 | >1000 | 302 | 21.5 |
| 321 | +++ | 322 | + | 323 | +++ |
| 324 | ++ | 325 | + | 326 | +++ |
| 327 | ++ | 328 | + | 329 | ++ |
| 343 | >1000 | 344 | 20.7 | 346 | +++ |
| 558 | ++ | 559 | + | 560 | ++ |
| 561 | ++ | 562 | + | 563 | ++ |
| 564 | ++ | 565 | + | 566 | ++ |
| 567 | ++ | 568 | + | 569 | ++ |
| 570 | ++ | 571 | + | 572 | ++ |
| 573 | ++ | 574 | + | 575 | ++ |
| 576 | +++ | 577 | >1000 | 578 | 29.2 |
| 579 | +++ | 580 | >1000 | 581 | 23.8 |
| 582 | ++ | 583 | + | 584 | ++ |
| 585 | + | 586 | + | 587 | ++ |
| 588 | + | 589 | + | 590 | + |
| 591 | + | 592 | + | 593 | ++ |
| 594 | ++ | 595 | + | 596 | ++ |
| 597 | ++ | 598 | + | 599 | +++ |
| 600 | +++ | 601 | + | 602 | +++ |
| 603 | ++ | 604 | + | 605 | +++ |
| 606 | ++ | 607 | + | 608 | +++ |
| 609 | ++ | 610 | + | 611 | +++ |
| 612 | ++ | 613 | + | 614 | +++ |
| 615 | +++ | 616 | + | 617 | +++ |
| 618 | ++ | 619 | + | 620 | ++ |
| 621 | ++ | 622 | + | 623 | ++ |
| 624 | ++ | 625 | + | 626 | ++ |
| 627 | + | 628 | + | 629 | + |
| 630 | ++ | 631 | + | 632 | ++ |
| 636 | + | 637 | + | 638 | ++ |
| 639 | + | 640 | ++ | 641 | + |
| 642 | ++ | 643 | + | 644 | + |
| 645 | +++ | 646 | >1000 | 647 | 19.9 |
| 648 | +++ | 649 | >1000 | 650 | 21.7 |
| 651 | +++ | 652 | >1000 | 653 | 25.9 |
| 654 | +++ | 655 | >1000 | 656 | 25.1 |
| 657 | +++ | 658 | >1000 | 659 | 20.3 |
| 660 | ++ | 661 | >1000 | 662 | 23.5 |
| 663 | +++ | 664 | >1000 | 665 | 20.6 |
| 666 | ++ | 667 | ++ | 668 | ++ |
| 669 | ++ | 670 | +++ | 671 | +++ |
| 672 | +++ | 673 | +++ | 674 | +++ |
| 675 | ++ | 676 | +++ | 677 | +++ |
| 678 | ++ | 679 | ++ | 680 | +++ |
| 681 | +++ | 682 | ++ | 683 | +++ |
| 684 | ++ | 685 | + | 686 | +++ |
| 687 | ++ | 688 | ++ | 689 | ++ |
| 690 | ++ | 691 | +++ | 692 | +++ |
| 693 | +++ | 694 | +++ | 695 | +++ |
| 696 | ++ | 697 | +++ | 698 | +++ |
| 699 | ++ | 700 | ++ | 701 | +++ |
| 702 | +++ | 703 | ++ | 704 | ++ |
| 705 | ++ | 706 | + | 707 | +++ |
| 708 | + | 709 | + | 710 | ++ |
| 711 | ++ | 712 | + | 713 | +++ |
| 714 | ++ | 715 | + | 716 | +++ |
| 717 | ++ | 718 | + | 719 | ++ |
| 720 | ++ | 721 | + | 722 | +++ |
| 723 | +++ | 724 | + | 725 | +++ |
| 726 | ++ | 727 | + | 728 | +++ |
| 729 | +++ | 730 | + | 731 | +++ |
| 732 | ++ | 733 | + | 734 | +++ |
| 735 | ++ | 736 | + | 737 | +++ |
| 738 | ++ | 739 | + | 740 | +++ |
| 741 | ++ | 742 | + | 743 | +++ |
| 744 | + | 745 | + | 746 | ++ |
| 747 | ++ | 748 | + | 749 | ++ |
| 750 | ++ | 751 | + | 752 | +++ |
| 753 | ++ | 754 | + | 755 | ++ |
| 756 | ++ | 757 | + | 758 | +++ |
| 759 | +++ | 760 | + | 761 | +++ |
| 762 | ++ | 763 | + | 764 | +++ |
| 984 | ++ | 985 | ++ | 986 | + |
| 987 | ++ | 988 | ++ | 989 | + |
| 990 | + | 991 | ++ | 992 | + |
| 1020 | ++ | 1021 | + | 1022 | +++ |
| 1023 | ++ | 1024 | + | 1025 | ++ |
| 1026 | ++ | 1027 | + | 1028 | ++ |
| 1029 | ++ | 1030 | + | 1031 | ++ |
| 1053 | ++ | 1054 | + | 1055 | +++ |
| 1056 | ++ | 1057 | + | 1058 | ++ |
| 1059 | ++ | 1060 | + | 1061 | ++ |
| TAK-981 | 53.2 | | | | |

+++ means that IC₅₀ is less than or equal to 50 nM
++ means that IC₅₀ is 50 nM to 200 nM
+ means that IC₅₀ is greater than 200 nM

TAK-981 is compound 1-263a in WO2016004136A1, and has the following chemical structure

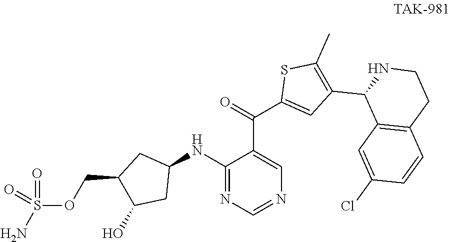 [SMILES: Cc1sc(C(=O)c2cncnc2N[C@@H]2C[C@H](COS(N)(=O)=O)[C@@H](O)C2)cc1[C@@H]1NCCc2ccc(Cl)cc21]

As can be seen from the data in Tables 2, 3, and 4, compared to TAK-981, most of the compounds of the present disclosure have a stronger inhibitory activity against SAE in the in vitro inhibitory activity assay against SAE.

### Biological Example 2. Assay for Compounds of the Present Disclosure in Promoting In Vitro Killing Activity of NK Cells on Tumor Cell OVCAR3

OVCAR3 cells and NK92MI cells were seeded separately at about 8000/well and at about 80000/well into a 96-well black transparent cell culture plate with 90 μL per well. The cells were incubated at 37° C. overnight for 24 h. The test compounds were 10-fold diluted to give a final concentration, and 10 μL of the diluted compounds was added to each well of the cell culture plate. The cells were incubated at 37° C. for 48 h. The OVCAR3 cell culture medium was discarded. The OVCAR3 cells were stained with 1 μM CalceinAM for 50 min, washed once with PBS, and then 100 μL of the culture solution was added. The NK92MI cells were mixed well and then gently added to the OVCAR3 cells. After about 4 h of co-incubation, the DMSO control group and the high concentration drug group were photographed using PICO, and the killing of OVCAR3 cells by NK92MI cells was observed. When a significant difference in killing between the two groups was observed, the co-incubation was stopped. The culture medium was discarded, and the cells were gently washed once with 100 μL of PBS. 100 μL of 4% PFA was added to each well, and the cells were immobilized at room temperature for 20 min, and then gently washed once with PBS. The fluorescence signal of the sample was detected through the FITC channel of a microplate reader. The fluorescence signal was compared to the DMSO group, and the inhibition rate and IC₅₀ were calculated.

### Biological Example 3. In Vivo Pharmacokinetic Experiment of Compound of the Present Disclosure

CD-1 female mice aged 6 to 8 weeks were intravenously administered at a dose of 2 mg/kg. The mice were fasted for at least 12 h before the administration and given food after the administration, and they were given ad libitum access to water during the experiment. On the day of the experiment, animals in the intravenous group were administered the corresponding compound by single injection via the tail vein at an administration volume of 0.2 mL/animal. The sample collection time was 0.083 h, 0.167 h, 0.5 h, 1 h, 2 h, 4 h, 8 h, and 24 h. About 150 μL of whole blood was collected through the submaxillary venous plexus at each time point (30 μL of whole blood was diluted with ultrapure water at a ratio of 1:3, and 120 μL of whole blood was centrifuged to obtain plasma) and used for concentration determination by high-performance liquid chromatography-tandem mass spectrometry (LC-MS/MS). All animals were sacrificed after the PK samples were collected at the last time point. The plasma concentration was processed using a non-compartmental model of Phoenix WinNonlin™ version 8.3 (Certara) pharmacokinetic software, and the pharmacokinetic parameters were calculated using a linear-log trapezoidal method. The in vivo pharmacokinetic results are shown in Tables 3 and 4 below.

TABLE 3
Results of in vivo pharmacokinetic evaluation of compound of the present disclosure

| | Administration | T_{1/2} | Cmax | C0 | AUClast | Vdss | CL |
|---|---|---|---|---|---|---|---|
| Compound | dose | (h) | (ng/mL) | (ng/mL) | (h*ng/mL) | (L/kg) | (mL/min/kg) |
| Compound | 2 mpk | 4.73 | 331 | 431 | 615 | 12.7 | 53.1 |
| 90 | | | | | | | |
| Compound | 2 mpk | 1.88 | 281 | 406 | 448 | 9.18 | 71.1 |
| 93 | | | | | | | |
| Compound | 2 mpk | 3.48 | 438 | 803 | 602 | 5.45 | 27.5 |
| 132 | | | | | | | |
| Compound | 1 mpk | 3.63 | 58 | 77 | 132 | 27.6 | 102.9 |
| 233 | | | | | | | |
| Compound | 2 mpk | 1.48 | 299 | 249 | 345 | 8.65 | 94.9 |
| 344 | | | | | | | |

TABLE 4
Plasma drug concentration and whole blood drug concentration
of compound of the present disclosure

| | Compound | Compound | Compound | Compound | Compound |
|---|---|---|---|---|---|
| | 90 | 93 | 132 | 233 | 344 |
| Administration dose | 2 mpk | 2 mpk | 2 mpk | 1 mpk | 2 mpk |
| Plasma Cmax (ng/mL) | 331 | 281 | 438 | 58 | 299 |
| Whole blood Cmax | 13433 | 13333 | 9273 | 4430 | 7776 |
| (ng/mL) | | | | | |
| Plasma AUClast | 615 | 448 | 602 | 132 | 345 |
| (h × ng/mL) | | | | | |
| Whole blood AUClast | 46567 | 25109 | 38193 | 30054 | 16992 |
| (h × ng/mL) | | | | | |
| Whole blood | 40.5 | 47.4 | 21.1 | 76.3 | 26.0 |
| Cmax/plasma Cmax | | | | | |
| ratio | | | | | |
| Whole blood | 75.7 | 56.0 | 63.4 | 227.6 | 49.2 |
| AUClast/plasma | | | | | |
| AUClast ratio | | | | | |

### Biological Example 4. In Vrvo Pharmacodynamic Study—Mouse MC38 Subcutaneous Xenograft Tumor Model

Each C57B3L/6 mouse was inoculated subcutaneously with 1×10⁶ MC38 cells, and when the tumors grew to 100-200 mm³, the vehicle was administered alone (intravenous injection, twice a week), the compound was administered alone (intravenous injection, twice a week), the anti-PD-i antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 5. In Vivo Pharmacodynamic Study —Mouse MC38 Subcutaneous Xenograft Tumor Model

Each C57BL/6 mouse was inoculated subcutaneously with 1×10⁶ MC38 cells, and when the tumors grew to 50-80 mm³, the vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 6. In Vivo Pharmacodynamic Study —Mouse MC38 Subcutaneous Xenograft Tumor Model

Each C57BL/6 mouse was inoculated subcutaneously with 1×10⁶ MC38 cells, and the treatment was administered immediately after the cell inoculation. The vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 7. In Vivo Pharmacodynamic Study —Mouse CT26 Subcutaneous Xenograft Tumor Model

Each BALB/c mouse was inoculated subcutaneously with 2×10⁵ CT26 cells, and when the tumors grew to 100-200 mm³, the vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 8. In Vivo Pharmacodynamic Study —Mouse CT26 Subcutaneous Xenograft Tumor Model

Each BALB/c mouse was inoculated subcutaneously with 2×10⁵ CT26 cells, and when the tumors grew to 50-80 mm³, the vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 9. In Vivo Pharmacodynamic Study —Mouse CT26 Subcutaneous Xenograft Tumor Model

Each BALB/c mouse was inoculated subcutaneously with 2×10⁵ CT26 cells, and the treatment was administered immediately after the cell inoculation. The vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 10. In Vivo Pharmacodynamic Study —Mouse A20 Subcutaneous Xenograft Tumor Model

Each BALB/c mouse was inoculated subcutaneously with 2×10⁶ A20 cells, and when the tumors grew to 100-200 mm³, the vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 11. In Vivo Pharmacodynamic Study —Mouse A20 Subcutaneous Xenograft Tumor Model

Each BALB/c mouse was inoculated subcutaneously with 2×10⁶A20 cells, and when the tumors grew to 50-80 mm³, the vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 12. In Vivo Pharmacodynamic Study —Mouse A20 Subcutaneous Xenograft Tumor Model

Each BALB/c mouse was inoculated subcutaneously with 2×10⁶ A20 cells, and the treatment was administered immediately after the cell inoculation. The vehicle was administered alone (intravenous injection, twice a week), the compound was administered alone (intravenous injection, twice a week), the anti-PD-1 antibody was administered alone (intravenous injection, once a week), the anti-VEGF antibody was administered alone (intraperitoneal injection, once a week), the anti-PD-1 antibody (intravenous injection, once a week) and the anti-VEGF antibody (intraperitoneal injection, once a week) were administered in combination, the compound (intravenous injection, twice a week) and the anti-PD-1 antibody (intravenous injection, once a week) were administered in combination, the compound (intravenous injection, twice a week) and the anti-VEGF antibody (intraperitoneal injection, once a week) were administered in combination, or the compound (intravenous injection, twice a week), the anti-PD-1 antibody (intravenous injection, once a week), and the anti-VEGF antibody (intraperitoneal injection, once a week) were administered in combination. The tumor volume was measured twice a week and at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group).

### Biological Example 13. In Vivo Pharmacodynamic Study —Mouse B16F10-OVA Subcutaneous Xenograft Tumor Model

On day 14, day 11, day 7, and day 4 prior to inoculation with B16F10-OVA cells, each C57BL/6 mouse was administered vehicle alone (intravenous injection), the compound alone (intravenous injection), Ovalbumin alone (intravenous injection), the anti-PD-1 antibody alone (intravenous injection), the anti-VEGF antibody alone (intraperitoneal injection), a combination of the compound (intravenous injection) and Ovalbumin (intravenous injection), a combination of the anti-PD-1 antibody (intravenous injection) and the anti-VEGF antibody (intraperitoneal injection), a combination of the compound (intravenous injection) and the anti-PD-1 antibody (intravenous injection), a combination of the compound (intravenous injection) and the anti-VEGF antibody (intraperitoneal injection), a combination of the compound (intravenous injection), the anti-PD-1 antibody (intravenous injection), and Ovalbumin (intravenous injection), a combination of the compound (intravenous injection), the anti-VEGF antibody (intraperitoneal injection), and Ovalbumin (intravenous injection), a combination of the compound (intravenous injection), the anti-PD-1 antibody (intravenous injection), and the anti-VEGF antibody (intraperitoneal injection), or a combination of the compound (intravenous injection), the anti-PD-1 antibody (intravenous injection), the anti-VEGF antibody (intraperitoneal injection), and Ovalbumin (intravenous injection). Each C57BL/6 mouse was inoculated subcutaneously with 0.3×10⁶ B16F 10-OVA cells, and the tumor volume was measured at the end of treatment. Tumor growth inhibition of the compound was calculated according to the following equation: tumor growth inhibition (TGI)=1−(tumor volume on day 20 in treatment group−tumor volume on day 1 in treatment group)/(tumor volume on day 20 in vehicle control group−tumor volume on day 1 in vehicle control group). Although specific embodiments of the present disclosure have been described above, it will be appreciated by those skilled in the art that these embodiments are merely illustrative and that many changes or modifications can be made to these embodiments without departing from the principles and spirit of the present disclosure. The protection scope of the present disclosure is therefore defined by the appended claims.

1. A compound of general formula (1) or an isomer thereof, a crystalline form thereof, a pharmaceutically acceptable salt thereof, a hydrate thereof, or a solvate thereof: 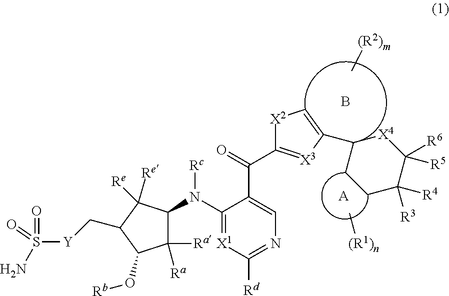
wherein in general formula (1):
Y is —O—, —CH₂—, or —N(H)—;
R^{a} is —H, —F, —NH₂, or —OH;
R^{a′} is —H or —F, and when R^{a} is —NH₂ or —OH, R^{a′} is —H;
R^{b} is —H or (C1-C4) alkyl;
R^{c} is —H or (C1-C4) alkyl;
R^{d} is —H, halogen, —CF₃, or (C1-C4) alkyl;
R^{e} and R^{e′} are each independently and optionally —H or halogen, and R^{e} and R^{e′} are not both —H;
X¹ is C(H), C(F), or N;
X² is S or O;
X³ is C(R^{x3}) or N;
R^{x3} is —H, halogen, or —CH₃;
X⁴ is S, O, C(R^{x41})(R^{x41′}), or N(R^{x42});
R^{x42} is —H, (C1-C4) alkyl or (C3-C5) cycloalkyl;
R^{x41} and R^{x41′} are each independently and optionally —H, halogen, —OH, —OR^{x411}, N(R^{x411})(R^{x412}) —CN, (C1-C6) alkyl, (C1-C6) haloalkyl, (C3-C9) cycloalkyl, or (C1-C6) alkoxy;
R^{x411} and R^{x412} are each independently and optionally —H, (C1-C4) alkyl, or (C3-C5) cycloalkyl, or R^{x411} and R^{x412} on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H or halogen;
R³ and R⁴ are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR³¹, —(CH₂)ᵣNR³¹R³², —OR³¹, —NR³¹R³², —CN, —C(O)NR³¹R³², —NR³²C(O)R³¹, —NR³²S(O)₂R³¹, —S(O)ₚR³¹, and —S(O)₂NR³¹R³²; or R³ and R⁴, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or R³ and the adjacent R⁵, together with the atom to which they are attached, can form a (C3-C9) cycloalkyl or (3- to 11-membered) heterocycloalkyl, wherein the (C3-C9) cycloalkyl or (3- to 11-membered) heterocycloalkyl may be each independently and optionally substituted with 1, 2, 3, or 4 of the following groups: —H, halogen, (C1-C6) alkyl, or (C1-C6) alkoxy; or when R³ and the adjacent R⁵ are both absent, an endocyclic double bond may be formed between the carbon atoms separately linked to R⁴ and R⁶; or R³ and R⁴ together form an oxo;
R⁵ and R⁶ are each independently and optionally —H, -D, —OH, —NH₂, —CN, (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl, wherein the (C1-C6) alkyl, (C1-C6) haloalkyl, (C2-C6) alkenyl, (C2-C6) alkynyl, (C3-C9) cycloalkyl, (C1-C6) alkoxy, (C6-C14) aryl, (3- to 11-membered) heterocycloalkyl, or (5- to 11-membered) heteroaryl may be each independently and optionally substituted with 1, 2, 3, or 4 of the following groups: —H, halogen, —OH, —(CH₂)ᵣOR³¹, —(CH₂)ᵣNR³¹R³², OR³¹, —NR³¹R³², —CN, —C(O)NR³¹R³², —NR³²C(O)R³¹, —NR³²S(O)₂R³¹, —S(O)ₚR³¹, and —S(O)₂NR³¹R³²; or R⁵ and R⁶, together with the carbon atom to which they are attached, can form a (4- to 7-membered) heterocycloalkyl or (C3-C6) cycloalkyl, wherein the (4- to 7-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or R⁵ and R⁶ together form an oxo;
ring A is (C6-C10) aryl or (5- to 10-membered) heteroaryl;
each R¹ is independently and optionally: —H, halogen, —OH, —NO₂, —NR³¹R³², —(CH₂)ᵣOR³¹, —(CH₂)ᵣNR³¹R³², —CN, (C1-C6) alkyl, (C1-C6) haloalkyl, (C1-C6) alkoxy, (C2-C6) alkenyl, (C2-C6) alkynyl, (C3-C8) cycloalkyl, —C(O)NR³¹R³², —NR³²C(O)R³¹, —NR³²S(O)₂R³¹, —S(O)ₚR³¹, or —S(O)₂NR³¹R³², wherein the (C1-C6) alkyl, (C1-C6) haloalkyl, (C1-C6) alkoxy, (C2-C6) alkenyl, (C2-C6) alkynyl, or (C3-C8) cycloalkyl may be each independently and optionally substituted with 1, 2, 3, or 4 of the following groups: —H, halogen, —OH, —(CH₂)ᵣOR³¹, —(CH₂)ᵣNR³¹R³², −OR³¹, —NR³¹R³², —CN, or (C1-C6) alkyl;
ring B is (C5-C7) cycloalkyl or (5- to 7-membered) heterocycloalkyl;
each R² is independently and optionally: —H, halogen, —OH, —NR³¹R³², —CN, (C1-C6) alkyl, (C1-C6) haloalkyl, (C1-C6) alkoxy, (C2-C6) alkenyl, (C2-C6) alkynyl, or (C3-C8) cycloalkyl; or two
R² on a same carbon atom, together with the carbon atom to which they are attached, can form (4- to 6-membered) heterocycloalkyl or (C3-C6) cycloalkyl, wherein the (4- to 6-membered) heterocycloalkyl or (C3-C6) cycloalkyl may be optionally substituted with 1, 2, 3, or 4 of the following groups: —H, halogen, (C1-C6) alkyl, or (C1-C6) alkoxy; or two R² on a same carbon atom together form an oxo;
R³¹ and R³² are each independently and optionally —H, (C1-C4) alkyl, or (C3-C5) cycloalkyl, or
R³¹ and R³² on a same nitrogen atom, together with the N atom to which they are attached, can form (3- to 6-membered) heterocycloalkyl, wherein the (3- to 6-membered) heterocycloalkyl may be optionally substituted with 1, 2, 3, or 4 of the following groups: —H and halogen; and
n is an integer of 0, 1, 2, 3, or 4, m is an integer of 0, 1, 2, 3, or 4, r is an integer of 0, 1, or 2, and
p is an integer of 0, 1, or 2.

2. The compound or the isomer thereof, the crystalline form thereof, the pharmaceutically acceptable salt thereof, the hydrate thereof, or the solvate thereof according to claim 1, wherein in the general formula (1), R^{d} is —H, —F, —CF₃, or —CH₃.

3. The compound or the isomer thereof, the crystalline form thereof, the pharmaceutically acceptable salt thereof, the hydrate thereof, or the solvate thereof according to claim 1, wherein in the general formula (1), R^{e} and R^{e′} are each independently and optionally —H or —F, and R^{e} and R^{e′} are not both —H.

4. The compound or the isomer thereof, the crystalline form thereof, the pharmaceutically acceptable salt thereof, the hydrate thereof, or the solvate thereof according to claim 1, wherein in the general formula (1), R^{x42} is —H, (C1-C3) alkyl, or (C3-C5) cycloalkyl.

5. The compound or the isomer thereof, the crystalline form thereof, the pharmaceutically acceptable salt thereof, the hydrate thereof, or the solvate thereof according to claim 4, wherein in the general formula (1), R^{x42} is —H,

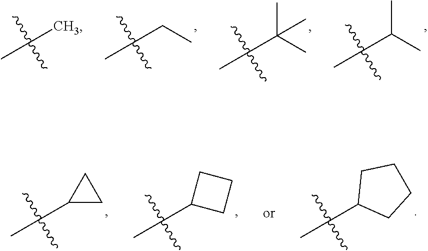

6. The compound or the isomer thereof, the crystalline form thereof, the pharmaceutically acceptable salt thereof, the hydrate thereof, or the solvate thereof according to claim 1, wherein in the general formula (1), R^{x41} and R^{x41′} are each independently and optionally —H, —F, —OH, —OCH₃, —N(CH₃)₂, —NH₂, —CN, 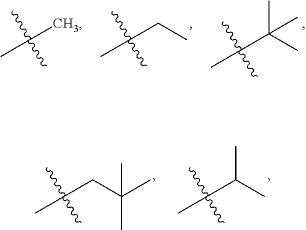
—CF₃, CH₂CF₃,

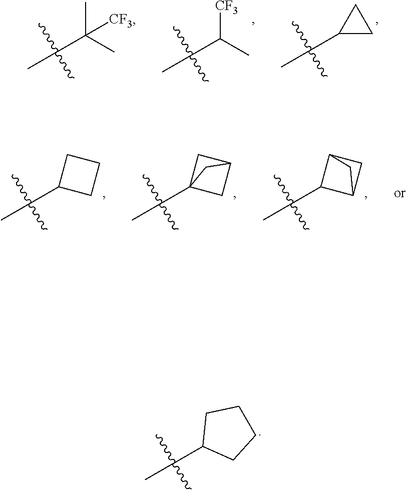

7. The compound or the isomer thereof, the crystalline form thereof, the pharmaceutically acceptable salt thereof, the hydrate thereof, or the solvate thereof according to claim 1, wherein in the general formula (1), R³ and R⁴ are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R³ and R⁴, together with the carbon atom to which they are attached, can form (4- to 6-membered) heterocycloalkyl or (C3-C4) cycloalkyl, wherein the (4- to 6-membered) heterocycloalkyl or (C3-C4) cycloalkyl may be optionally substituted with 1, 2, 3, or 4 of the following groups: —H, —F, 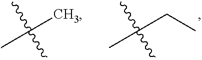
or —OCH₃; or R³ and the adjacent R⁵, together with the atom to which they are attached, form a (C3-C6) cycloalkyl or (3- to 6-membered) heterocycloalkyl, wherein the (C3-C6) cycloalkyl or (3- to 6-membered) heterocycloalkyl may be each independently and optionally substituted with 1, 2, 3, or 4 of the following groups: —H, —F, 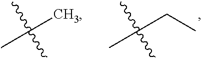
or —OCH₃; or when R³ and the adjacent R⁵ are both absent, an endocyclic double bond may be formed between the carbon atoms separately linked to R⁴ and R⁶; or R³ and R⁴ together form an oxo.

8. The compound or the isomer thereof, the crystalline form thereof, the pharmaceutically acceptable salt thereof, the hydrate thereof, or the solvate thereof according to m claim 1, wherein in the general formula (1), R⁵ and R⁶ are each independently and optionally —H, -D, —OH, —NH₂, —CN, (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl, wherein the (C1-C3) alkyl, (C1-C3) haloalkyl, (C2-C4) alkenyl, (C2-C4) alkynyl, (C3-C5) cycloalkyl, (C1-C3) alkoxy, phenyl, (4- to 6-membered) heterocycloalkyl, or (5- to 6-membered) heteroaryl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂; or R⁵ and R⁶, together with the carbon atom to which they are attached, can form a (4- to 6-membered) heterocycloalkyl or (C3-C4) cycloalkyl, wherein the (4- to 6-membered) heterocycloalkyl or (C3-C4) cycloalkyl may be optionally substituted with 1, 2, 3, or 4 of the following groups: —H, —F, 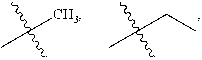
or —OCH₃; or R⁵ and R⁶ together form an oxo.

9. The compound or the isomer thereof, the crystalline form thereof, the pharmaceutically acceptable salt thereof, the hydrate thereof, or the solvate thereof according to claim 1, wherein in the general formula (1), ring A is phenyl or (5- to 6-membered) heteroaryl.

10. The compound or the isomer thereof, the crystalline form thereof, the pharmaceutically acceptable salt thereof, the hydrate thereof, or the solvate thereof according to claim 9, wherein in the general formula (1), ring A is:

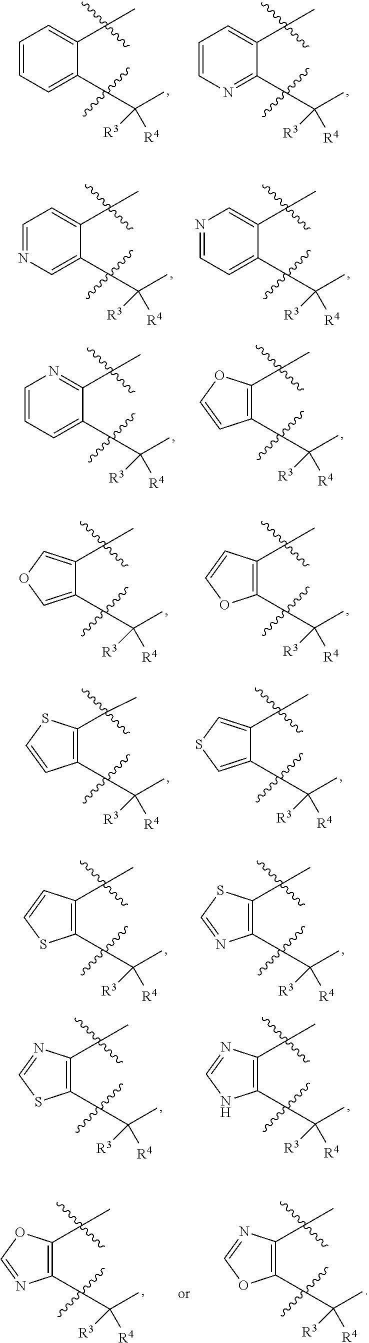

11. The compound or the isomer thereof, the crystalline form thereof, the pharmaceutically acceptable salt thereof, the hydrate thereof, or the solvate thereof according to claim 1, wherein in the general formula (1), each R¹ is independently and optionally: —H, —F, —Cl, —Br, —I, —OH, —NO₂, —N(CH₃)₂, —NH₂, —CH₂OCH₃, —CH₂N(CH₃)₂, —CN, (C1-C3) alkyl, (C1-C3) haloalkyl, (C1-C3) alkoxy, (C2-C4) alkenyl, (C2-C4) alkynyl, (C3-C6) cycloalkyl, —C(O)N(CH₃)₂, —NCH₃C(O)CH₃, —NHC(O)CH₃, —NCH₃S(O)₂CH₃, —NHS(O)₂CH₃, —SCH₃, —S(O)₂CH₃, —S(O)₂NH₂, and —S(O)₂N(CH₃)₂, wherein the (C1-C3) alkyl, (C1-C3) haloalkyl, (C1-C3) alkoxy, (C2-C4) alkenyl, (C2-C4) alkynyl, or (C3-C6) cycloalkyl may be each independently and optionally substituted with 1, 2, 3, or 4 of the following groups: —H, —F, —OH, —CH₂OCH₃, —CH₂N(CH₃)₂, —OCH₃, —N(CH₃)₂, —NH₂, —CN,

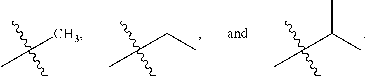

12. The compound or the isomer thereof, the crystalline form thereof, the pharmaceutically acceptable salt thereof, the hydrate thereof, or the solvate thereof according to claim 11, wherein in the general formula (1), each R¹ is independently: —H, —F, —Cl, —Br, —I, —OH, —NO₂, —N(CH₃)₂, —NH₂, —CH₂OCH₃, —CH₂N(CH₃)₂, —CN, —C(O)N(CH₃)₂, —NCH₃C(O)CH₃, —NHC(O)CH₃, —NCH₃ S(O)₂CH₃, —NHS(O)₂CH₃, —SCH₃, —S(O)₂CH₃, —S(O)₂NH₂, —S(O)₂N(CH₃)₂, 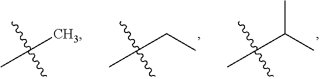
CF₃, —CH₂CF₃, 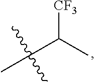 [SMILES: *C(C)C(F)(F)F]
—OCH₃, —OCH₂CH₃, —OCH(CH₃)₂,

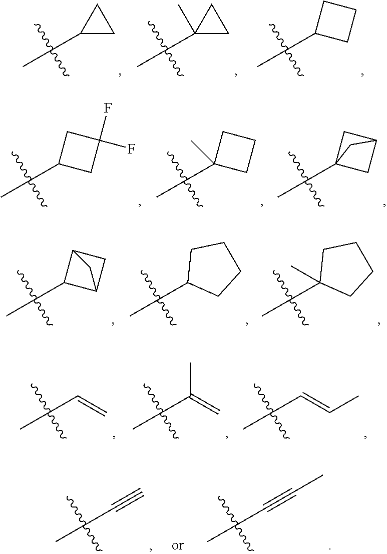

13. The compound or the isomer thereof, the crystalline form thereof, the pharmaceutically acceptable salt thereof, the hydrate thereof, or the solvate thereof according to claim 1, wherein in the general formula (1), ring B is (C5-C6) cycloalkyl or (5- to 6-membered) heterocycloalkyl.

14. The compound or the isomer thereof, the crystalline form thereof, the pharmaceutically acceptable salt thereof, the hydrate thereof, or the solvate thereof according to claim 13, wherein in the general formula (1), the structural unit 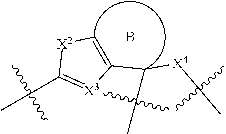
is

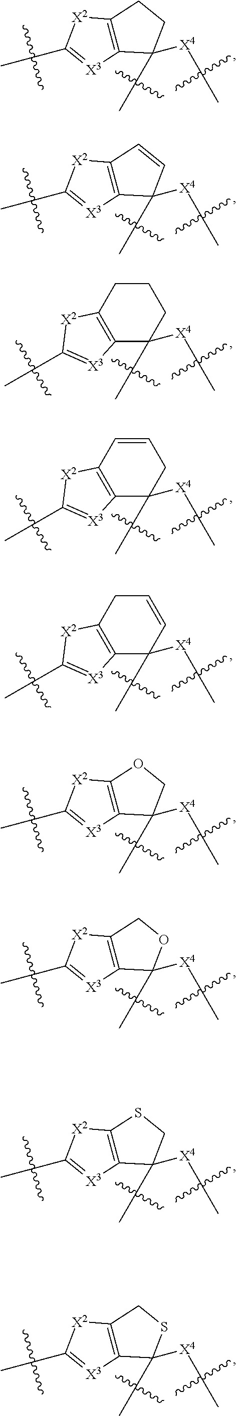

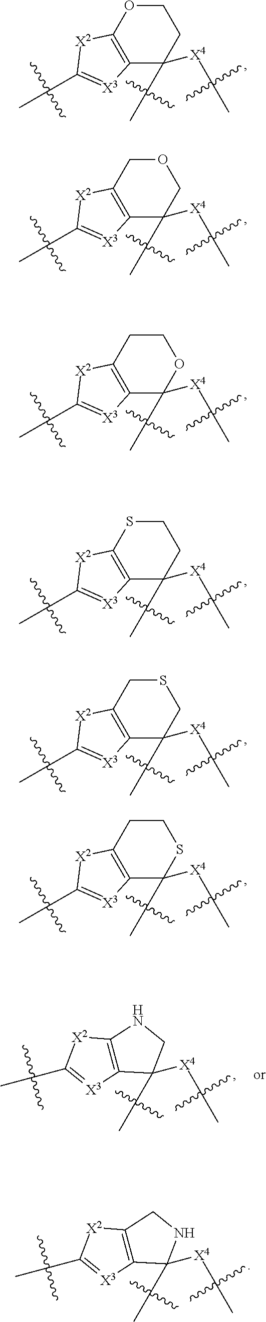

15. The compound or the isomer thereof, the crystalline form thereof, the pharmaceutically acceptable salt thereof, the hydrate thereof, or the solvate thereof according to claim 13, wherein in the general formula (1), the structural unit 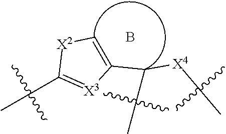
is:

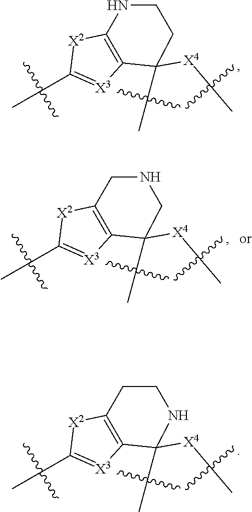

16. The compound or the isomer thereof, the crystalline form thereof, the pharmaceutically acceptable salt thereof, the hydrate thereof, or the solvate thereof according to claim 1, wherein in the general formula (1), each R² is independently and optionally: —H, —F, —Cl, —Br, —I, —OH, —N(CH₃)₂, —NH₂, —CN, (C1-C3) alkyl, (C1-C3) haloalkyl, (C1-C3) alkoxy, (C2-C4) alkenyl, (C2-C4) alkynyl, or (C3-C5) cycloalkyl; or
two R² on a same carbon atom, together with the carbon atom to which they are attached, can form (4- to 5-membered) heterocycloalkyl or (C3-C5) cycloalkyl, wherein the (4- to 5-membered) heterocycloalkyl or (C3-C5) cycloalkyl may be optionally substituted with 1, 2, 3, or 4 of the following groups: —H, —F, 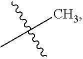
and —OCH₃; or two R² on a same carbon atom together form an oxo.

17. The compound or the isomer thereof, the crystalline form thereof, the pharmaceutically acceptable salt thereof, the hydrate thereof, or the solvate thereof according to claim 16, wherein in the general formula (1), each R² is independently and optionally: —H, —F, —Cl, —Br, —I, —OH, —N(CH₃)₂, —NH₂, —CN, 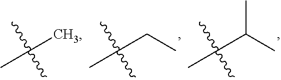
—CF₃, —CH₂CF₃, 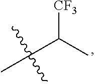 [SMILES: *C(C)C(F)(F)F]
—OCH₃, —OCH₂CH₃, —OCH(CH₃)₂,

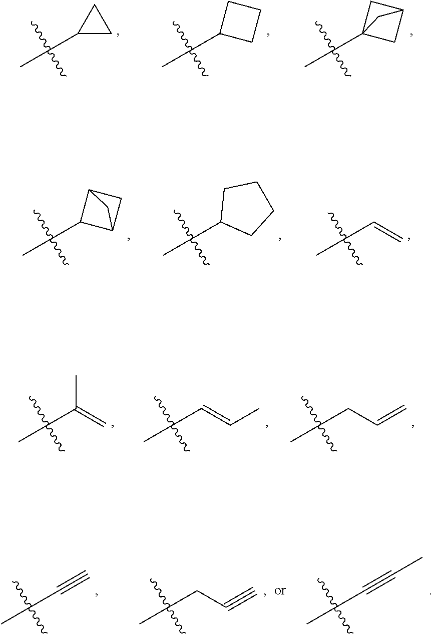

18. The compound or the isomer thereof, the crystalline form thereof, the pharmaceutically acceptable salt thereof, the hydrate thereof, or the solvate thereof according to claim 1, wherein in the general formula (1), the structural unit 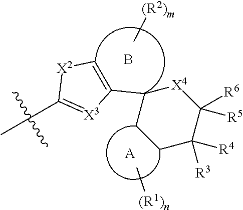
is:

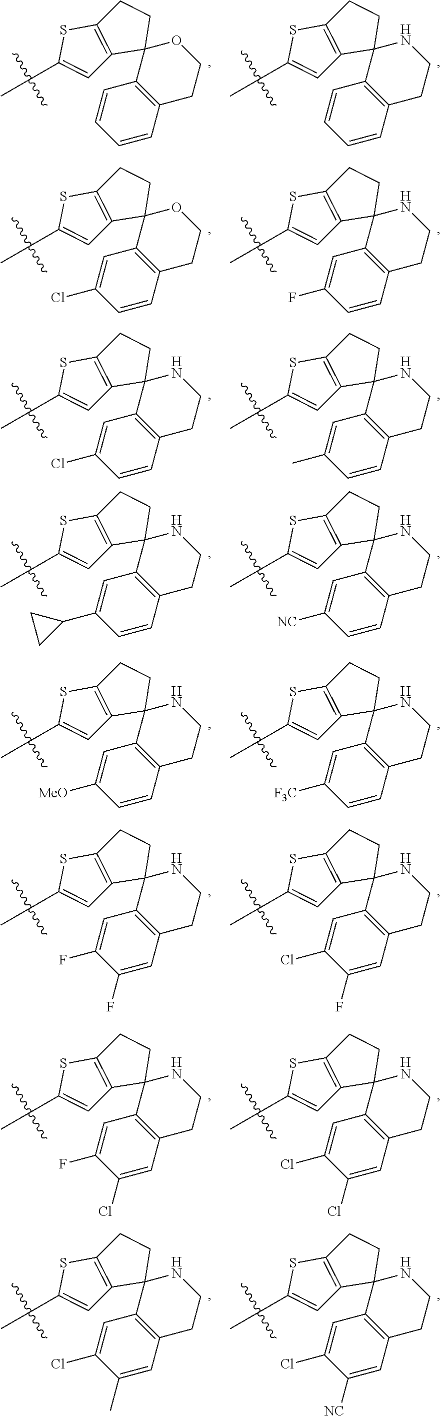

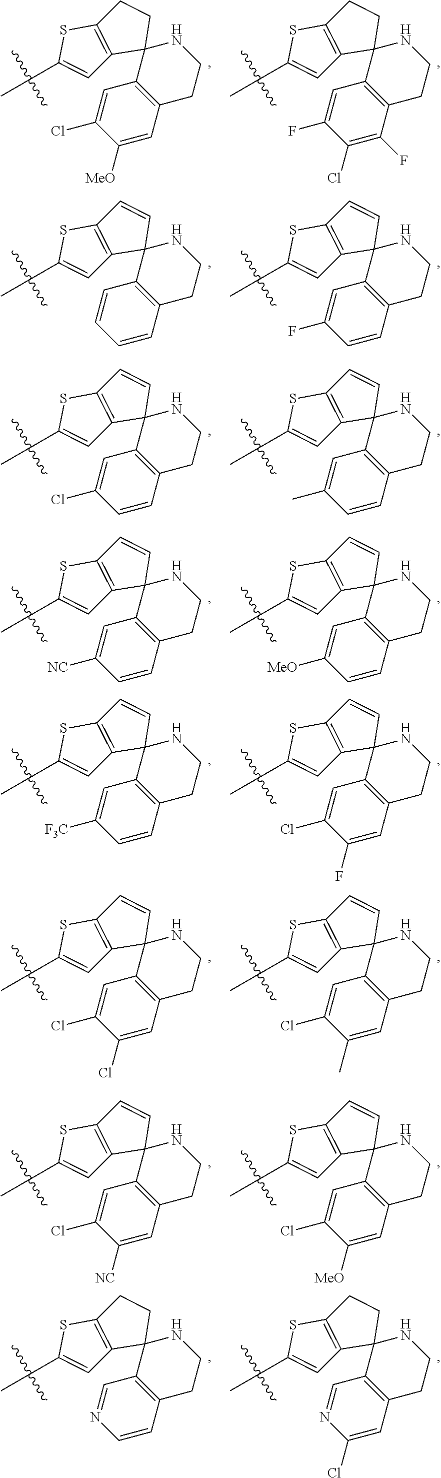

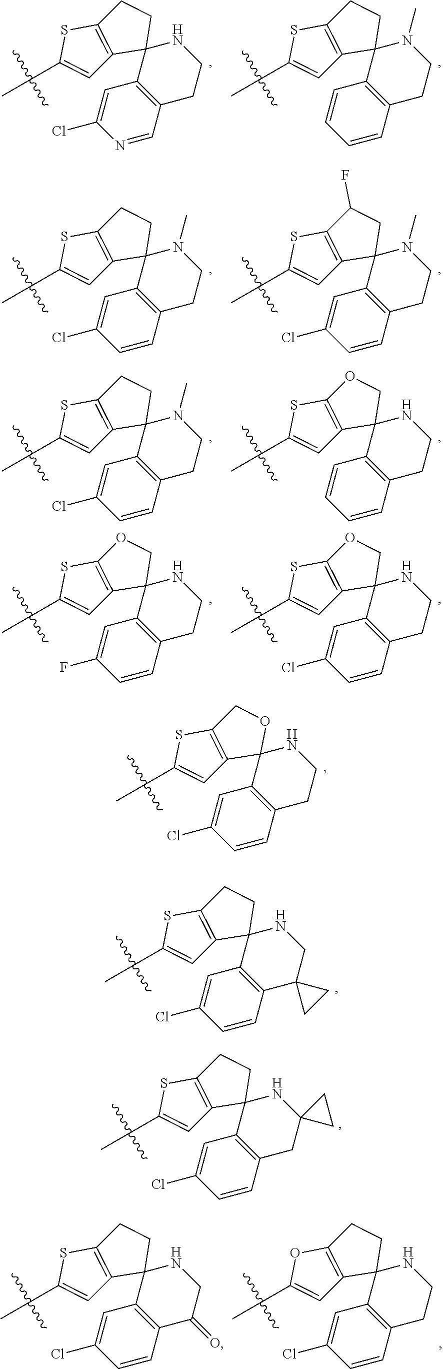

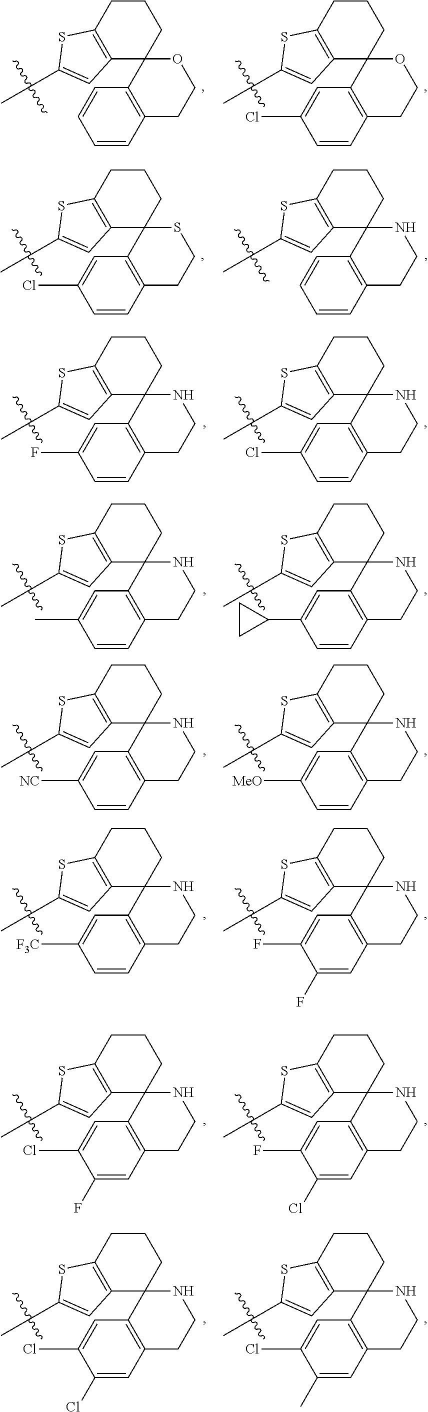

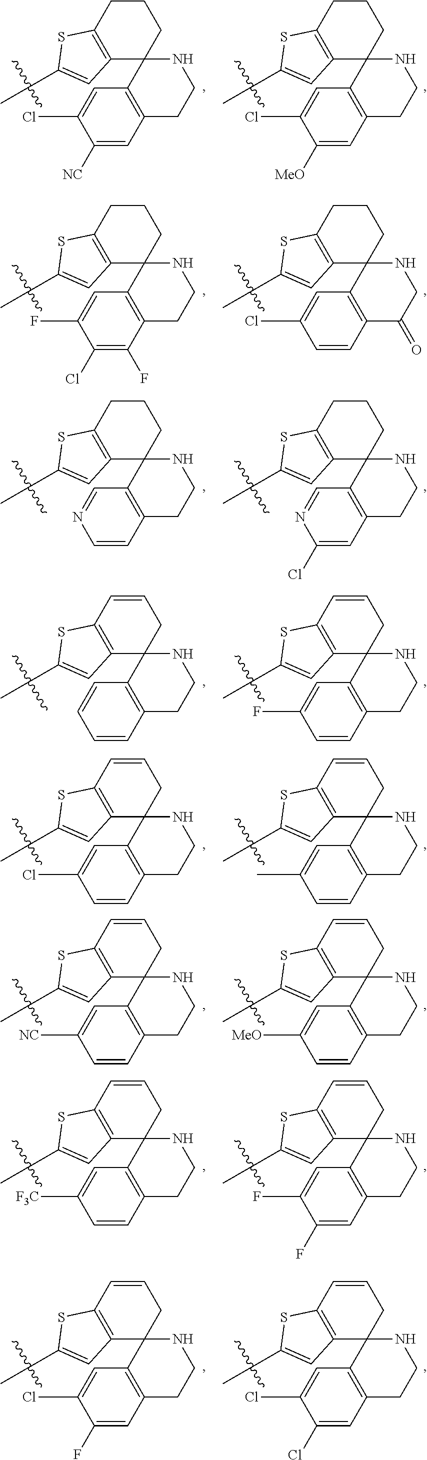

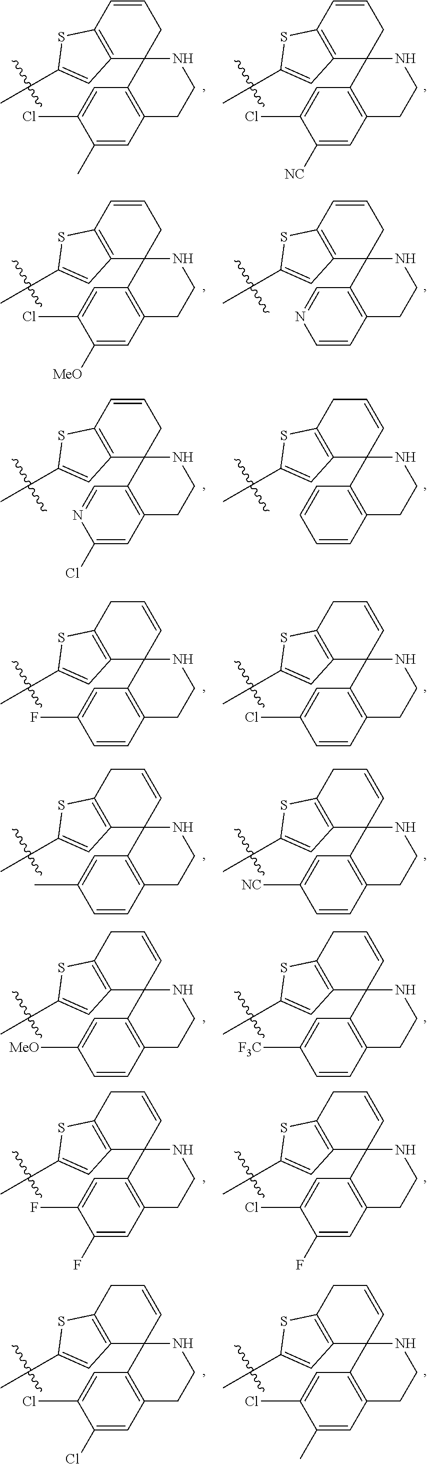

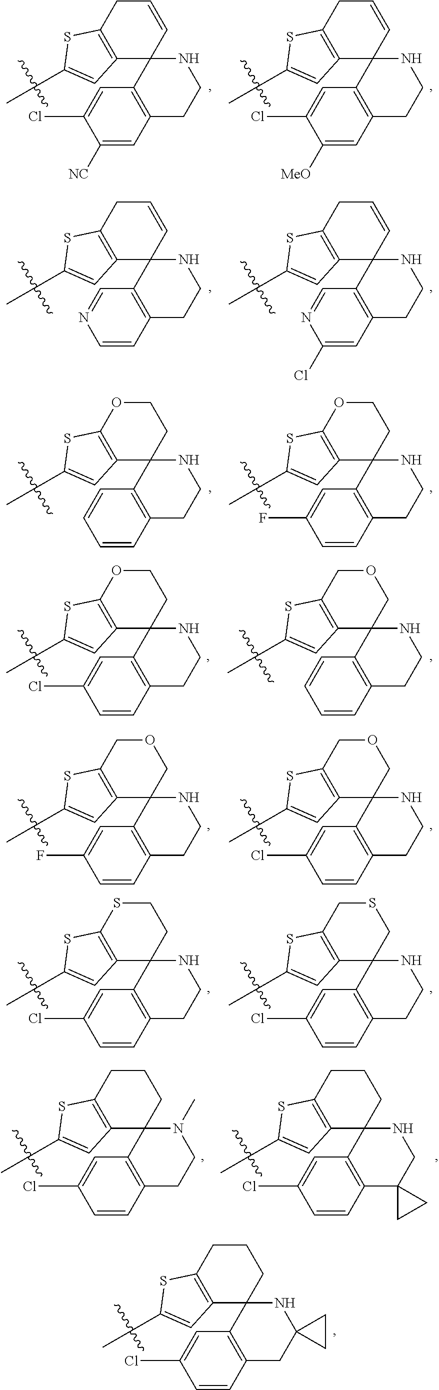

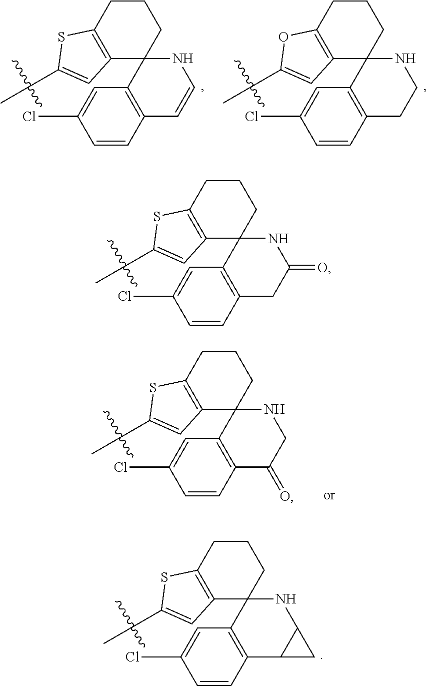

19. The compound or the isomer thereof, the crystalline form thereof, the pharmaceutically acceptable salt thereof, the hydrate thereof, or the solvate thereof according to claim 1, wherein in the general formula (1), the structural unit 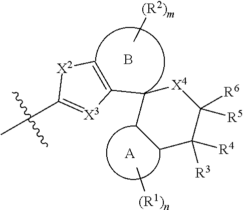
is:

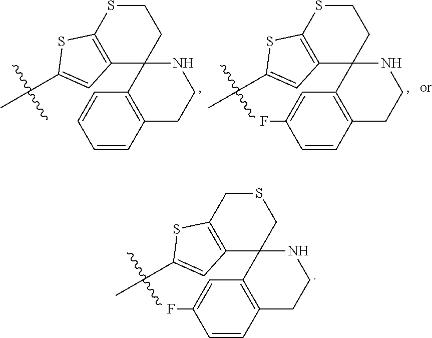

20. The compound or the isomer thereof, the crystalline form thereof, the pharmaceutically acceptable salt thereof, the hydrate thereof, or the solvate thereof according to claim 1, wherein in the general formula (1), the structural unit 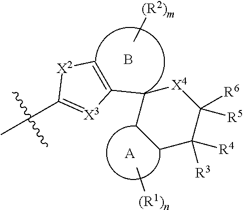
is:

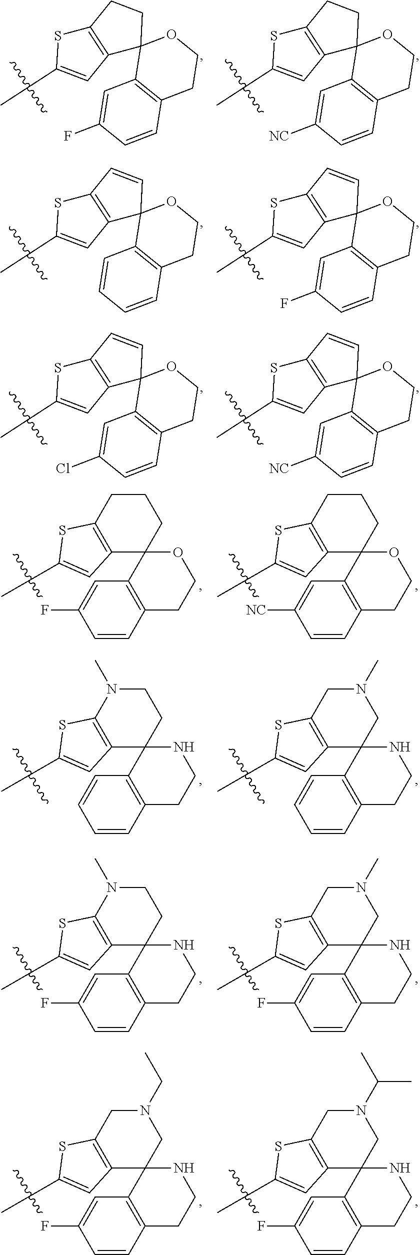

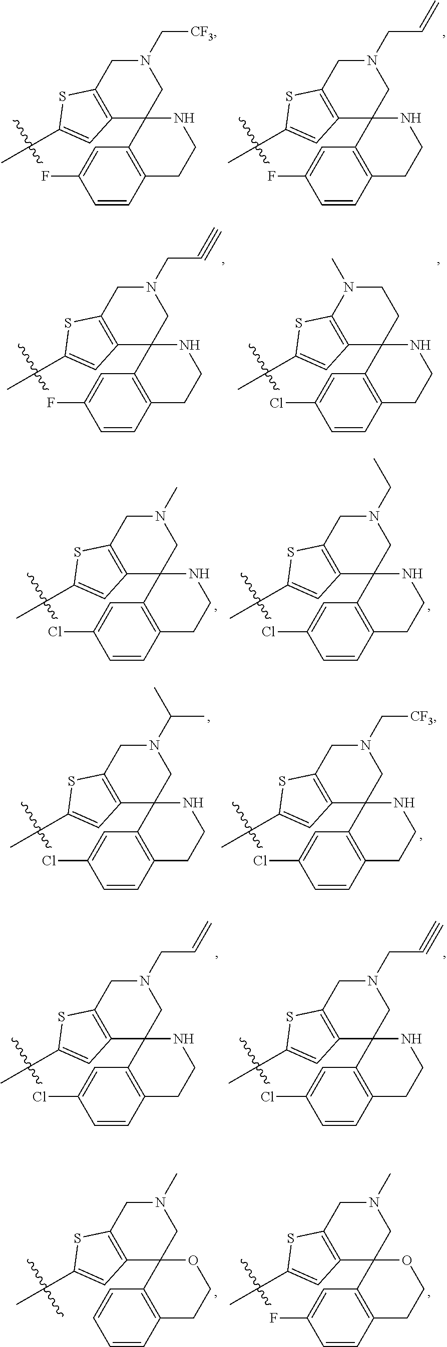

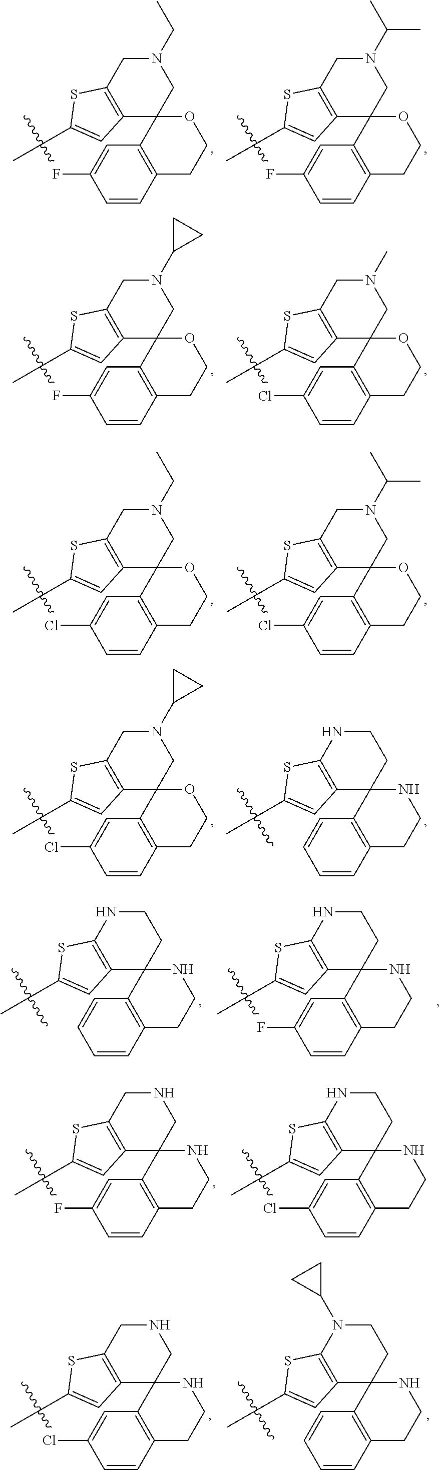

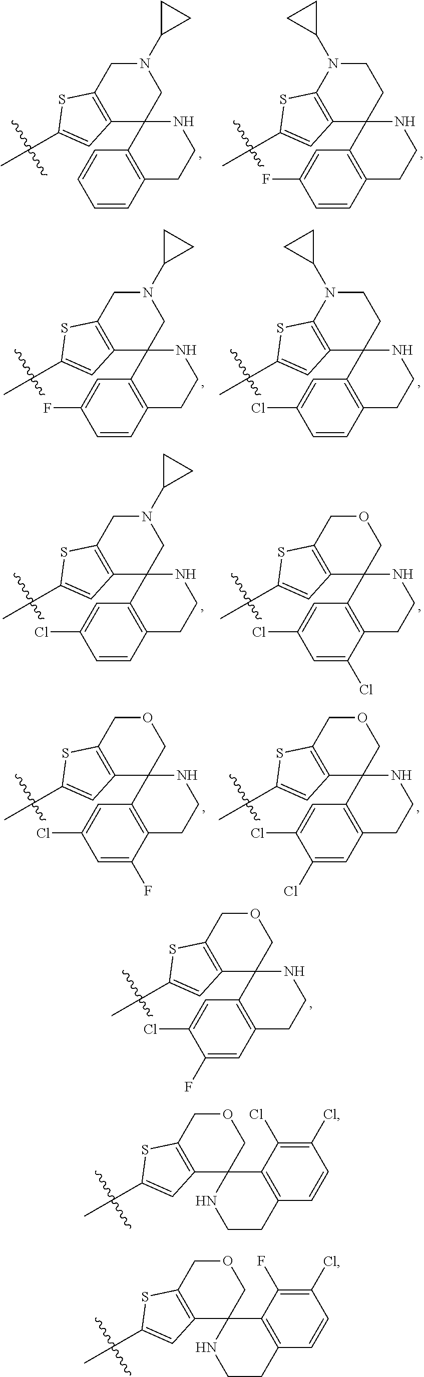

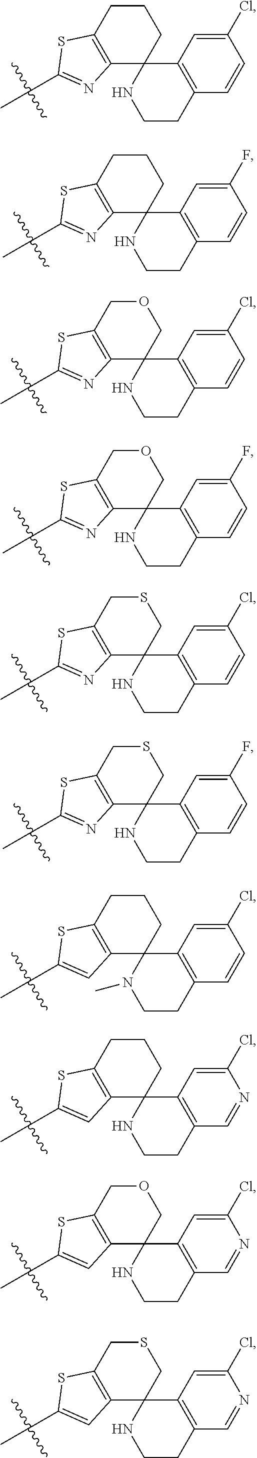

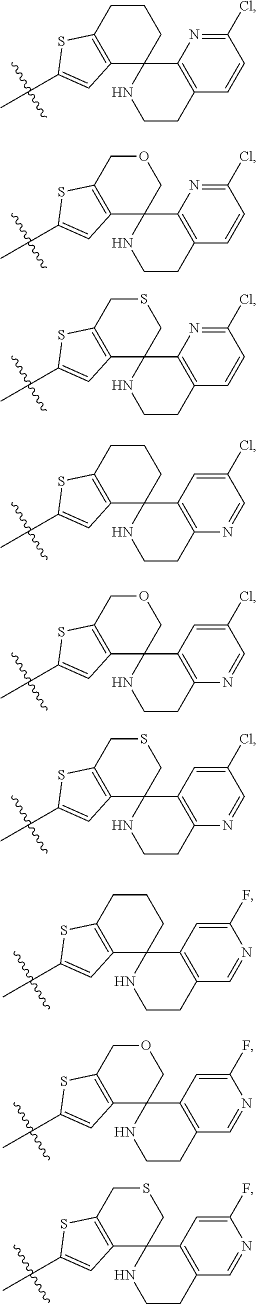

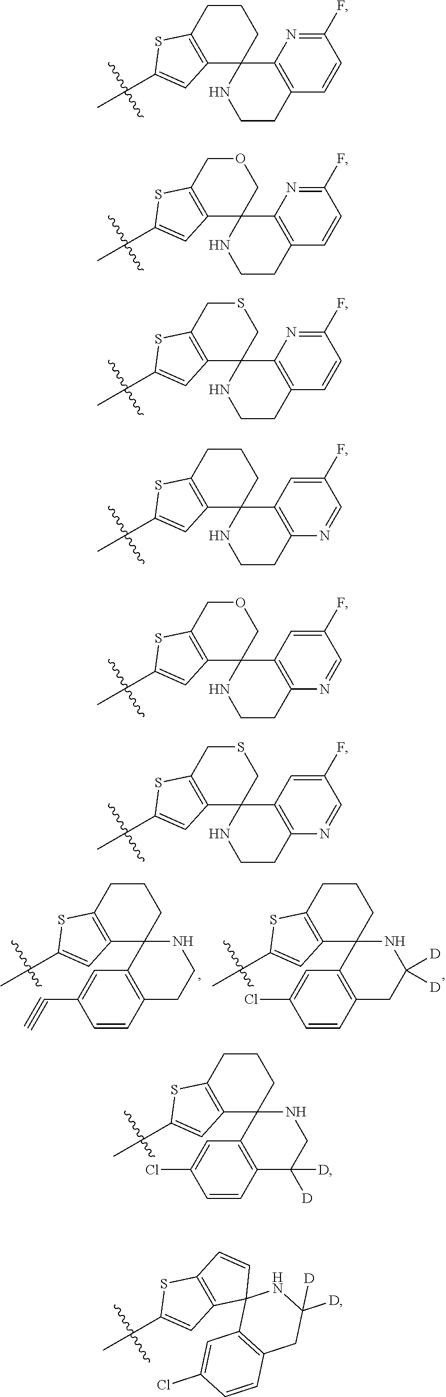

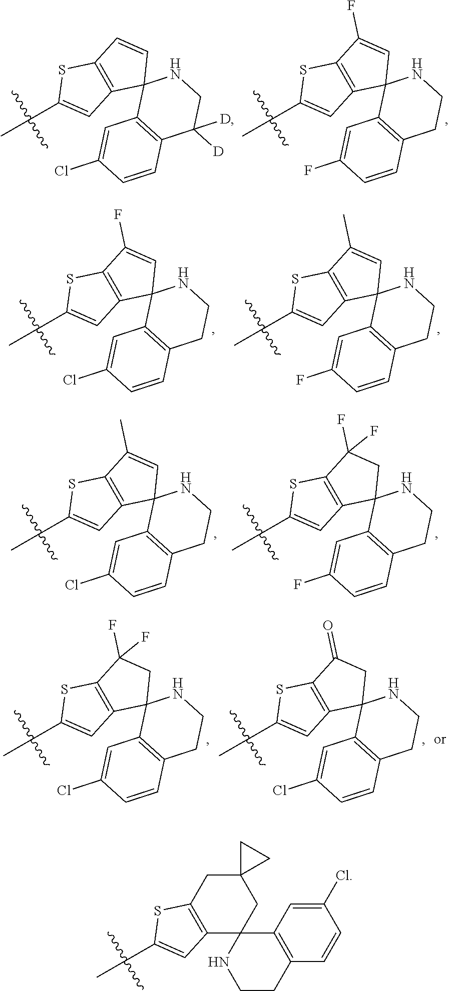

21-46. (canceled)

47. The compound or the isomer thereof, the crystalline form thereof, the pharmaceutically acceptable salt thereof, the hydrate thereof, or the solvate thereof according to claim 1, wherein the compound has one of the following structures:

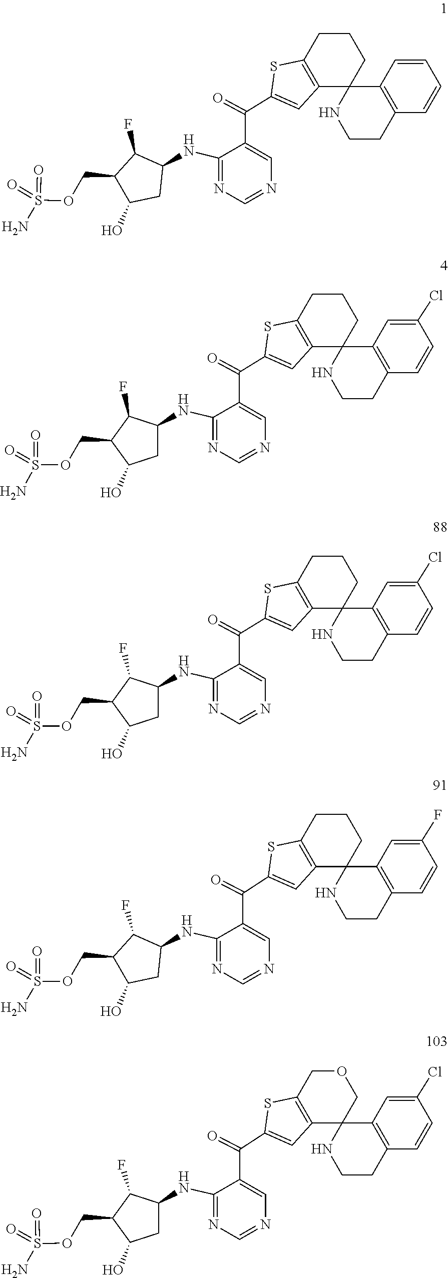 [SMILES: NS(=O)(=O)OC[C@H]1[C@@H](F)[C@@H](Nc2ncncc2C(=O)c2cc3c(s2)CCCC32NCCc3ccccc32)C[C@@H]1O ; NS(=O)(=O)OC[C@H]1[C@@H](F)[C@@H](Nc2ncncc2C(=O)c2cc3c(s2)CCCC32NCCc3ccc(Cl)cc32)C[C@@H]1O ; NS(=O)(=O)OC[C@H]1[C@H](F)[C@@H](Nc2ncncc2C(=O)c2cc3c(s2)CCCC32NCCc3ccc(Cl)cc32)C[C@@H]1O ; NS(=O)(=O)OC[C@H]1[C@H](F)[C@@H](Nc2ncncc2C(=O)c2cc3c(s2)CCCC32NCCc3ccc(F)cc32)C[C@@H]1O ; NS(=O)(=O)OC[C@H]1[C@H](F)[C@@H](Nc2ncncc2C(=O)c2cc3c(s2)COCC32NCCc3ccc(Cl)cc32)C[C@@H]1O]

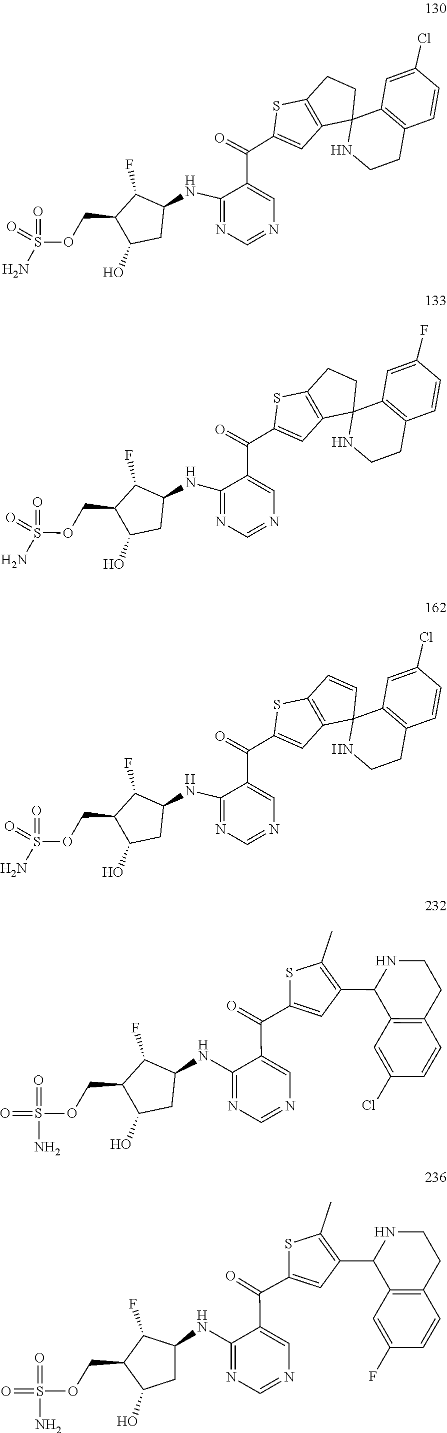 [SMILES: NS(=O)(=O)OC[C@H]1[C@H](F)[C@@H](Nc2ncncc2C(=O)c2cc3c(s2)CCC32NCCc3ccc(Cl)cc32)C[C@@H]1O ; NS(=O)(=O)OC[C@H]1[C@H](F)[C@@H](Nc2ncncc2C(=O)c2cc3c(s2)CCC32NCCc3ccc(F)cc32)C[C@@H]1O ; NS(=O)(=O)OC[C@H]1[C@H](F)[C@@H](Nc2ncncc2C(=O)c2cc3c(s2)C=CC32NCCc3ccc(Cl)cc32)C[C@@H]1O ; Cc1sc(C(=O)c2cncnc2N[C@H]2C[C@H](O)[C@@H](COS(N)(=O)=O)[C@@H]2F)cc1C1NCCc2ccc(Cl)cc21 ; Cc1sc(C(=O)c2cncnc2N[C@H]2C[C@H](O)[C@@H](COS(N)(=O)=O)[C@@H]2F)cc1C1NCCc2ccc(F)cc21]

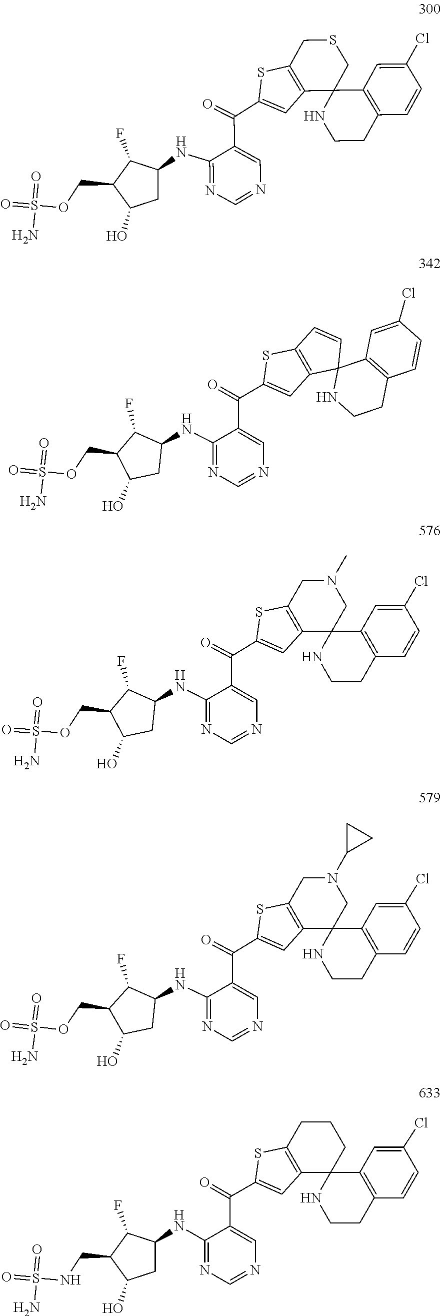 [SMILES: NS(=O)(=O)OC[C@H]1[C@H](F)[C@@H](Nc2ncncc2C(=O)c2cc3c(s2)CSCC32NCCc3ccc(Cl)cc32)C[C@@H]1O ; NS(=O)(=O)OC[C@H]1[C@H](F)[C@@H](Nc2ncncc2C(=O)c2cc3c(s2)C=CC32NCCc3ccc(Cl)cc32)C[C@@H]1O ; CN1Cc2sc(C(=O)c3cncnc3N[C@H]3C[C@H](O)[C@@H](COS(N)(=O)=O)[C@@H]3F)cc2C2(C1)NCCc1ccc(Cl)cc12 ; NS(=O)(=O)OC[C@H]1[C@H](F)[C@@H](Nc2ncncc2C(=O)c2cc3c(s2)CN(C2CC2)CC32NCCc3ccc(Cl)cc32)C[C@@H]1O ; NS(=O)(=O)NC[C@H]1[C@H](F)[C@@H](Nc2ncncc2C(=O)c2cc3c(s2)CCCC32NCCc3ccc(Cl)cc32)C[C@@H]1O]

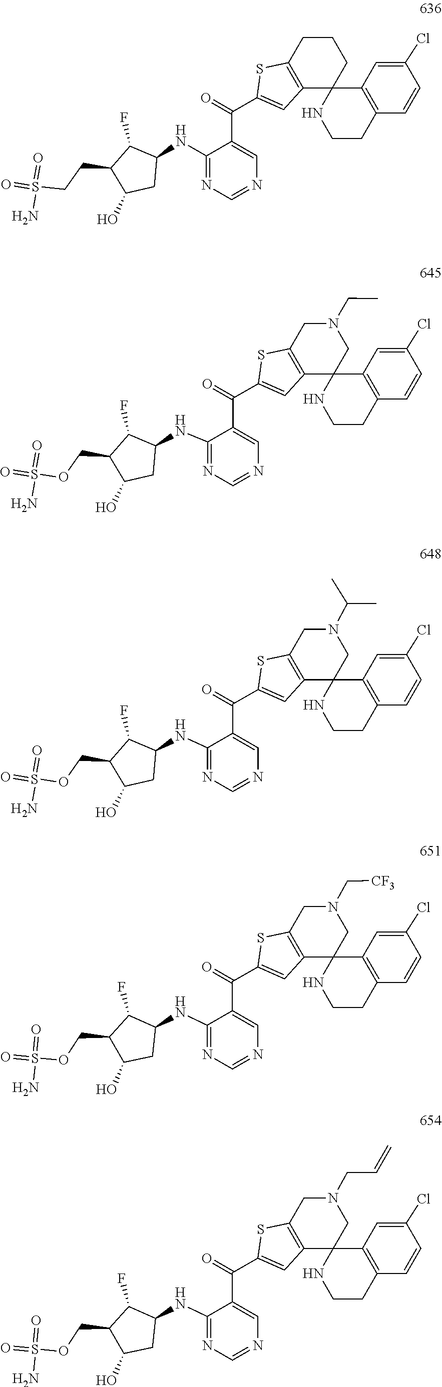 [SMILES: NS(=O)(=O)CC[C@H]1[C@H](F)[C@@H](Nc2ncncc2C(=O)c2cc3c(s2)CCCC32NCCc3ccc(Cl)cc32)C[C@@H]1O ; CCN1Cc2sc(C(=O)c3cncnc3N[C@H]3C[C@H](O)[C@@H](COS(N)(=O)=O)[C@@H]3F)cc2C2(C1)NCCc1ccc(Cl)cc12 ; CC(C)N1Cc2sc(C(=O)c3cncnc3N[C@H]3C[C@H](O)[C@@H](COS(N)(=O)=O)[C@@H]3F)cc2C2(C1)NCCc1ccc(Cl)cc12 ; NS(=O)(=O)OC[C@H]1[C@H](F)[C@@H](Nc2ncncc2C(=O)c2cc3c(s2)CN(CC(F)(F)F)CC32NCCc3ccc(Cl)cc32)C[C@@H]1O ; C=CCN1Cc2sc(C(=O)c3cncnc3N[C@H]3C[C@H](O)[C@@H](COS(N)(=O)=O)[C@@H]3F)cc2C2(C1)NCCc1ccc(Cl)cc12]

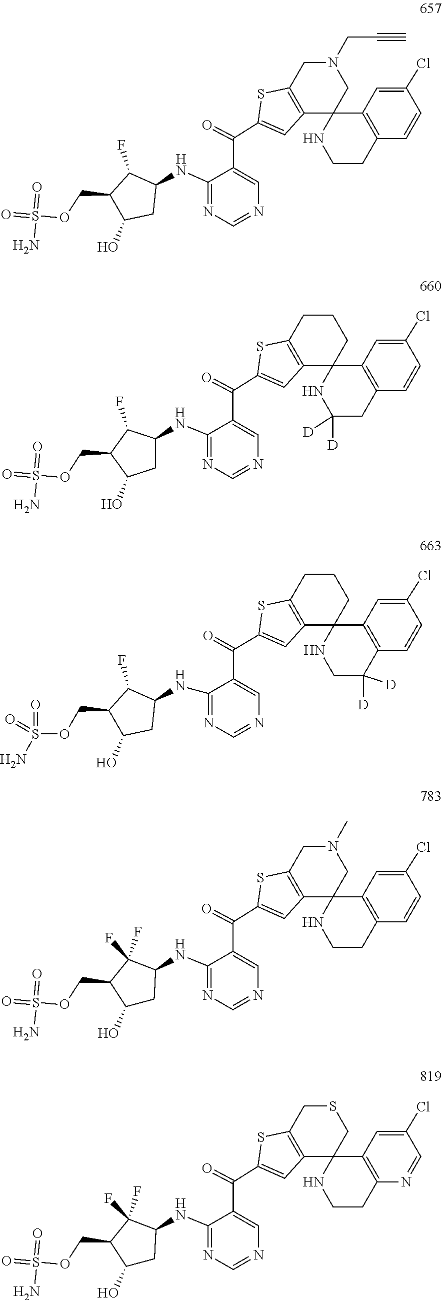

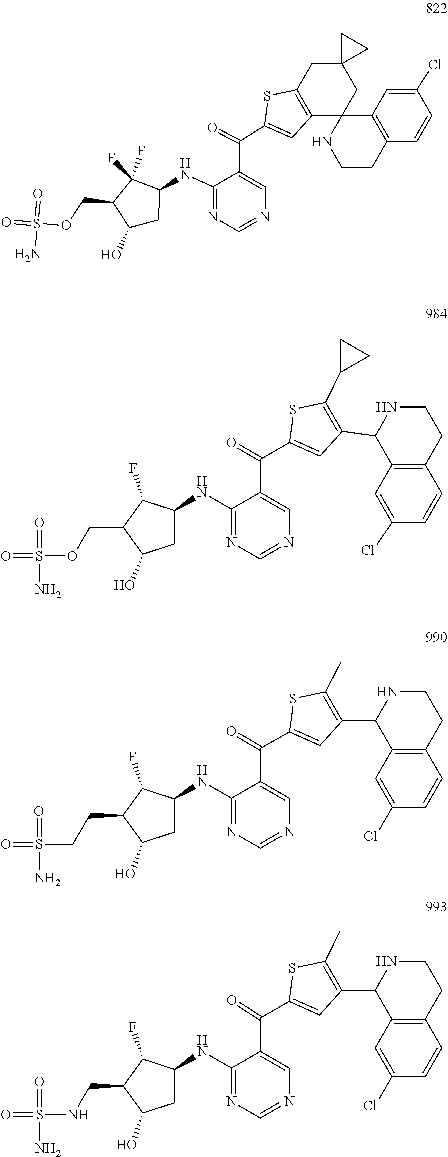

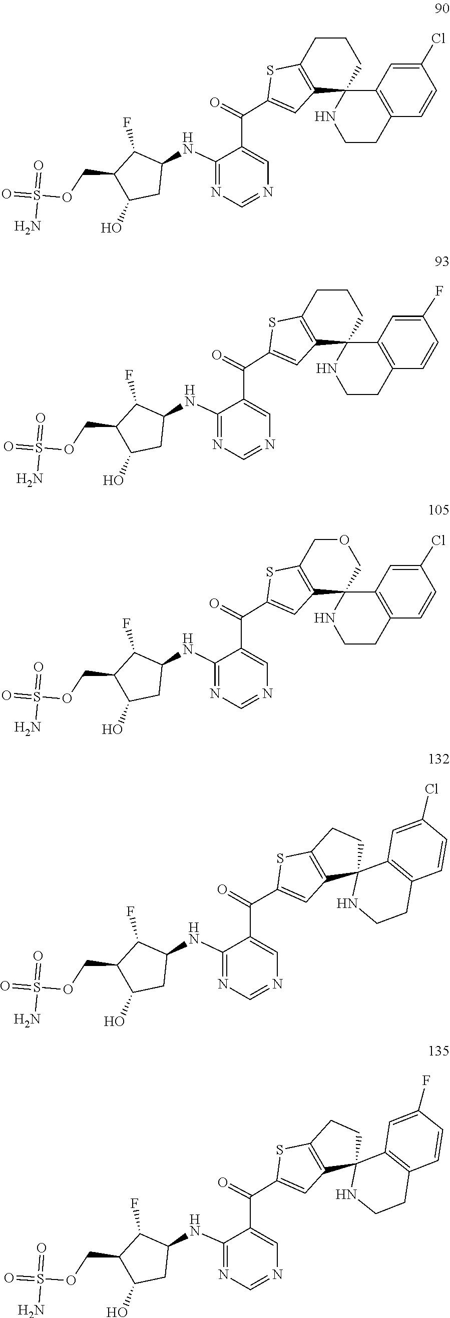 [SMILES: NS(=O)(=O)OC[C@H]1[C@H](F)[C@@H](Nc2ncncc2C(=O)c2cc3c(s2)CCC[C@]32NCCc3ccc(Cl)cc32)C[C@@H]1O ; NS(=O)(=O)OC[C@H]1[C@H](F)[C@@H](Nc2ncncc2C(=O)c2cc3c(s2)CCC[C@]32NCCc3ccc(F)cc32)C[C@@H]1O ; NS(=O)(=O)OC[C@H]1[C@H](F)[C@@H](Nc2ncncc2C(=O)c2cc3c(s2)COC[C@]32NCCc3ccc(Cl)cc32)C[C@@H]1O ; NS(=O)(=O)OC[C@H]1[C@H](F)[C@@H](Nc2ncncc2C(=O)c2cc3c(s2)CC[C@]32NCCc3ccc(Cl)cc32)C[C@@H]1O ; NS(=O)(=O)OC[C@H]1[C@H](F)[C@@H](Nc2ncncc2C(=O)c2cc3c(s2)CC[C@]32NCCc3ccc(F)cc32)C[C@@H]1O]

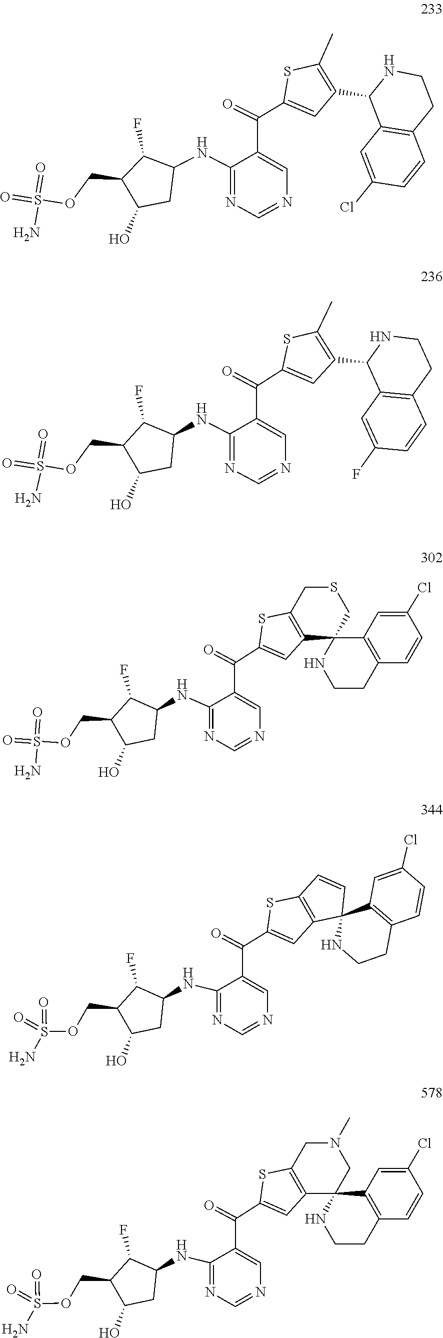 [SMILES: Cc1sc(C(=O)c2cncnc2NC2C[C@H](O)[C@@H](COS(N)(=O)=O)[C@@H]2F)cc1[C@@H]1NCCc2ccc(Cl)cc21 ; Cc1sc(C(=O)c2cncnc2N[C@H]2C[C@H](O)[C@@H](COS(N)(=O)=O)[C@@H]2F)cc1[C@@H]1NCCc2ccc(F)cc21 ; NS(=O)(=O)OC[C@H]1[C@H](F)[C@@H](Nc2ncncc2C(=O)c2cc3c(s2)CSC[C@]32NCCc3ccc(Cl)cc32)C[C@@H]1O ; NS(=O)(=O)OC[C@H]1[C@H](F)[C@@H](Nc2ncncc2C(=O)c2cc3c(s2)C=C[C@]32NCCc3ccc(Cl)cc32)C[C@@H]1O ; CN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12]

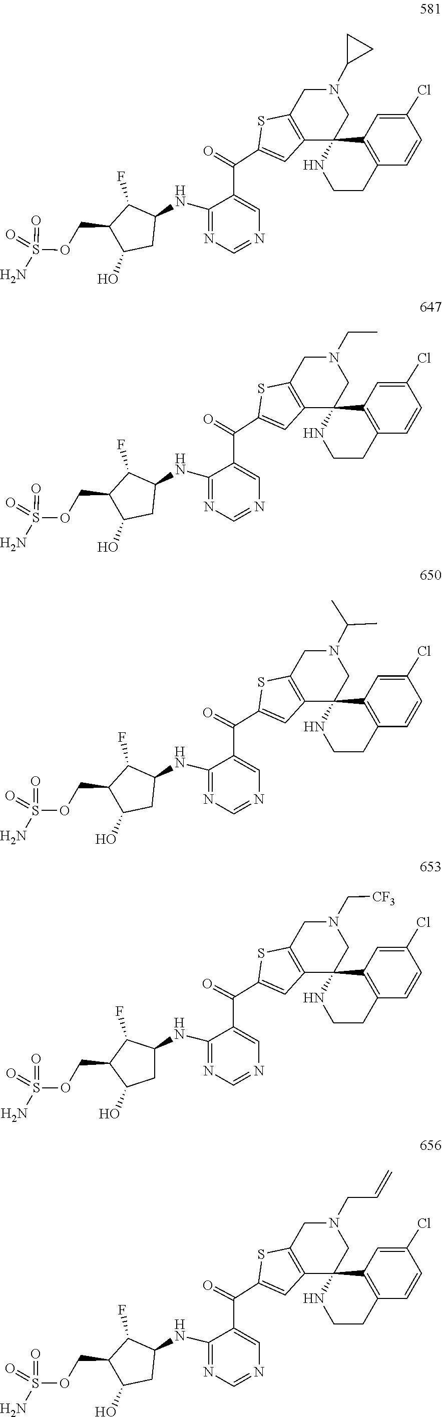

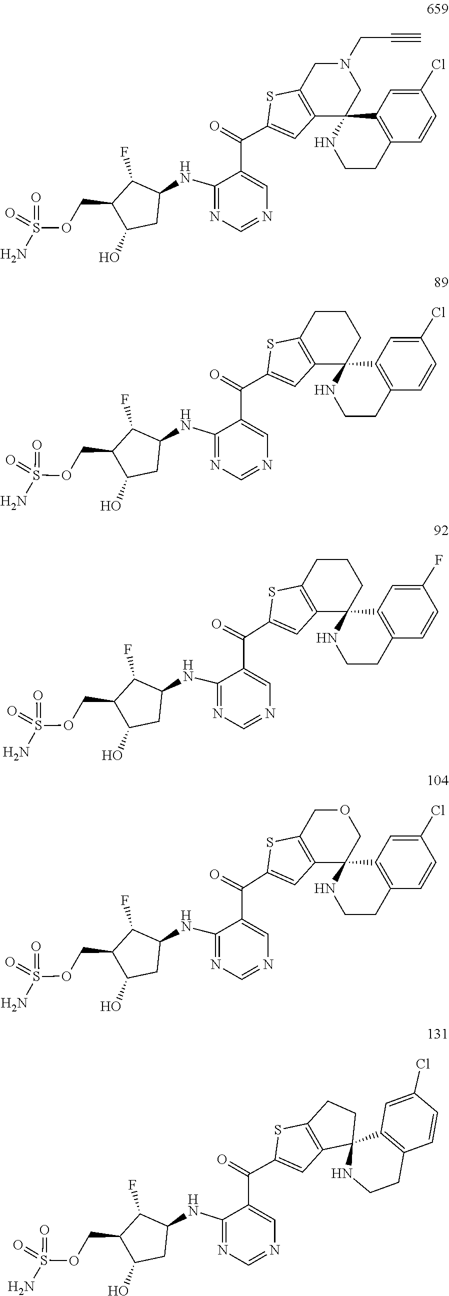 [SMILES: C#CCN1Cc2sc(C(=O)c3cncnc3N[C@H]3C[C@H](O)[C@@H](COS(N)(=O)=O)[C@@H]3F)cc2[C@]2(C1)NCCc1ccc(Cl)cc12 ; NS(=O)(=O)OC[C@H]1[C@H](F)[C@@H](Nc2ncncc2C(=O)c2cc3c(s2)CCC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)CCC[C@@]32NCCc3ccc(F)cc32)C[C@@H]1O ; NS(=O)(=O)OC[C@H]1[C@H](F)[C@@H](Nc2ncncc2C(=O)c2cc3c(s2)COC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)CC[C@@]32NCCc3ccc(Cl)cc32)C[C@@H]1O]

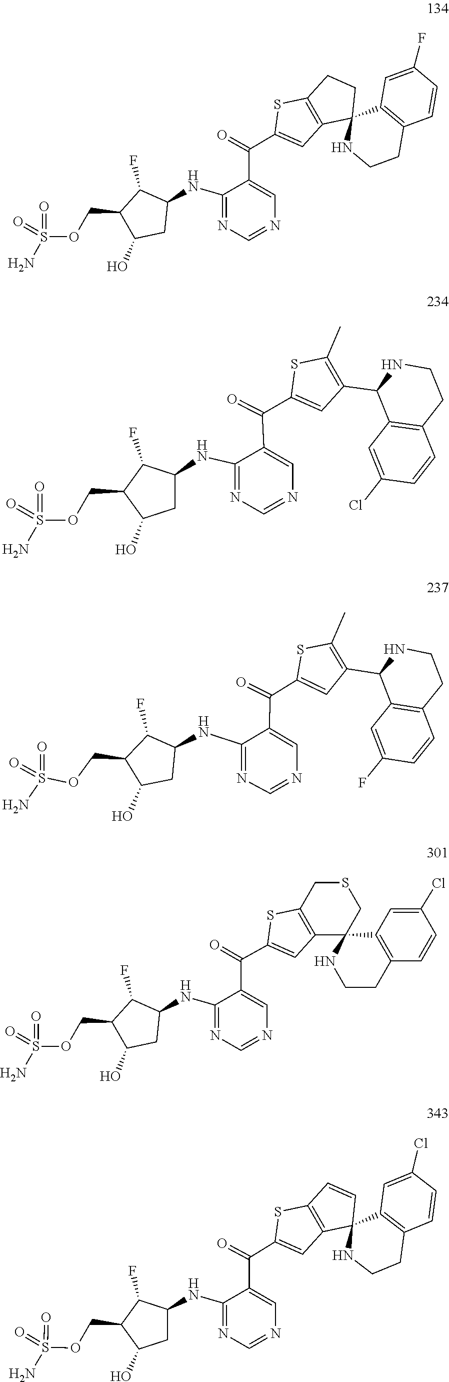 [SMILES: NS(=O)(=O)OC[C@H]1[C@H](F)[C@@H](Nc2ncncc2C(=O)c2cc3c(s2)CC[C@@]32NCCc3ccc(F)cc32)C[C@@H]1O ; Cc1sc(C(=O)c2cncnc2N[C@H]2C[C@H](O)[C@@H](COS(N)(=O)=O)[C@@H]2F)cc1[C@H]1NCCc2ccc(Cl)cc21 ; Cc1sc(C(=O)c2cncnc2N[C@H]2C[C@H](O)[C@@H](COS(N)(=O)=O)[C@@H]2F)cc1[C@H]1NCCc2ccc(F)cc21 ; NS(=O)(=O)OC[C@H]1[C@H](F)[C@@H](Nc2ncncc2C(=O)c2cc3c(s2)CSC[C@@]32NCCc3ccc(Cl)cc32)C[C@@H]1O ; NS(=O)(=O)OC[C@H]1[C@H](F)[C@@H](Nc2ncncc2C(=O)c2cc3c(s2)C=C[C@@]32NCCc3ccc(Cl)cc32)C[C@@H]1O]

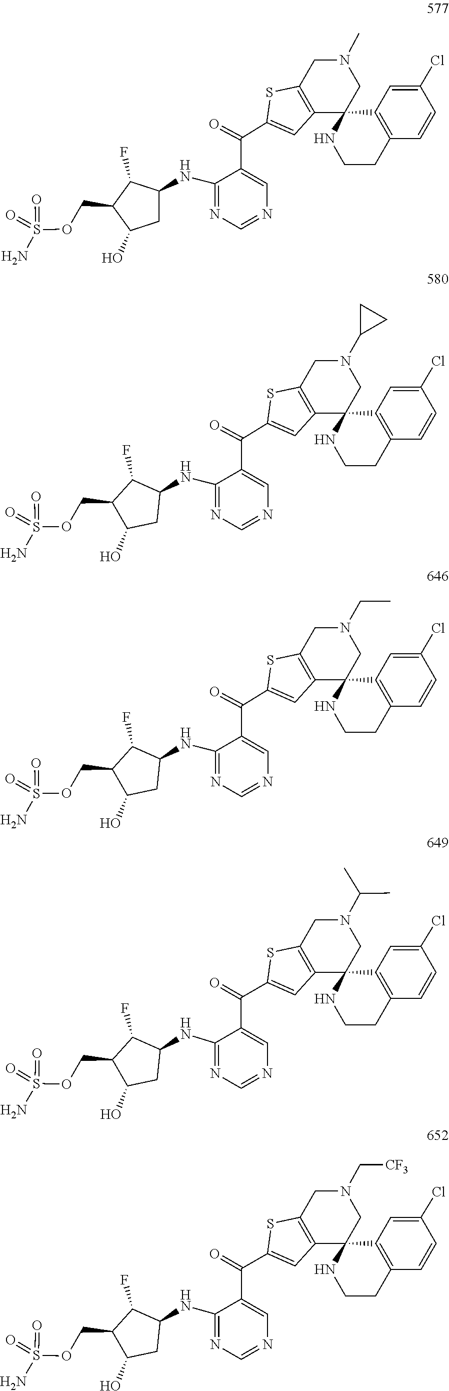

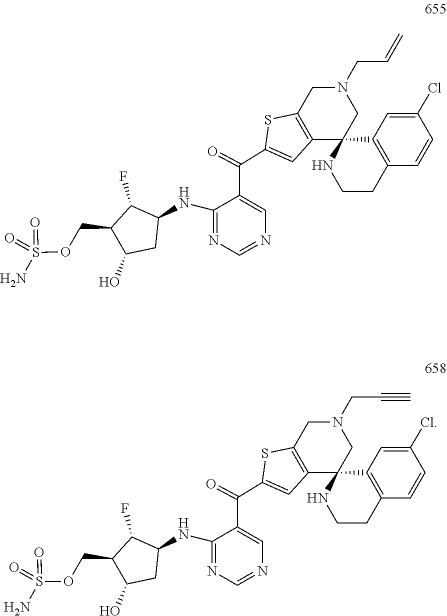 [SMILES: C=CCN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12 ; C#CCN1Cc2sc(C(=O)c3cncnc3N[C@H]3C[C@H](O)[C@@H](COS(N)(=O)=O)[C@@H]3F)cc2[C@@]2(C1)NCCc1ccc(Cl)cc12]

48. A pharmaceutical composition, comprising a pharmaceutically acceptable excipient or carrier, and the compound or the isomer thereof, the crystalline form thereof, the pharmaceutically acceptable salt thereof, the hydrate thereof, or the solvate thereof according to claim 1 as an active ingredient.
